(19) United States
(12) Patent Application Publication
(10) Pub. No.: US 2024/0368095 A1
(43) Pub. Date: Nov. 7, 2024

(54) HETEROCYCLIC COMPOUND AND ORGANIC LIGHT-EMITTING DEVICE COMPRISING SAME

(71) Applicant: LT MATERIALS CO., LTD., Yongin-si, Gyeonggi-do (KR)

(72) Inventors: Woo Jeong CHAE, Yongin-si (KR); Jun Tae MO, Yongin-si (KR); Dong Jun KIM, Yongin-si (KR)

(73) Assignee: LT MATERIALS CO., LTD., Yongin-si, Gyeonggi-do (KR)

(21) Appl. No.: 18/563,604

(22) PCT Filed: Oct. 28, 2022
(86) PCT No.: PCT/KR2022/016705

§ 371 Date: Nov. 22, 2023

(30) Foreign Application Priority Data
Dec. 8, 2021 (KR) 10-2021-0174862

(51) Int. Cl. C07D 277/60; C07B 59/00; C07D 263/52; C07D 413/10; C07D 413/14; C07D 417/10; C07D 417/14; C09K 11/06; H10K 85/60

(52) U.S. Cl. CPC C07D 277/60; C07B 59/004; C07D 263/52; C07D 413/10; C07D 413/14; C07D 417/10; C07D 417/14; C09K 11/06; H10K 85/622; H10K 85/633; H10K 85/6572; H10K 85/6574; H10K 85/6576; C07B 2200/05

(57) ABSTRACT

The present disclosure relates to a heterocyclic compound represented by Chemical Formula 1 and an organic light emitting device including the same.

### TECHNICAL FIELD

This application claims priority to and the benefits of Korean Patent Application No. 10-2021-0174862, filed with the Korean Intellectual Property Office on Dec. 8, 2021, the entire contents of which are incorporated herein by reference.

The present disclosure relates to a heterocyclic compound and an organic light emitting device comprising the same.

### BACKGROUND ART

An organic light emitting device is one type of self-emissive display devices, and has advantages of having a wide viewing angle and a high response speed as well as having an excellent contrast.

The organic light emitting device has a structure of disposing an organic thin film between two electrodes. When a voltage is applied to the organic light emitting device having such a structure, electrons and holes injected from the two electrodes bind and pair in the organic thin film, and then light emits as these annihilate. The organic thin film may be formed in a single layer or a multilayer as necessary.

A material of the organic thin film may have a light emitting function as necessary. For example, as a material of the organic thin film, compounds each capable of forming a light emitting layer themselves alone may be used, or compounds each capable of performing a role of a host or a dopant of a host-dopant-based light emitting layer may also be used. In addition thereto, compounds capable of performing roles of hole injection, hole transport, electron blocking, hole blocking, electron transport, electron injection and the like may also be used as a material of the organic thin film.

Development of an organic thin film material has been continuously required for enhancing performance, lifetime or efficiency of an organic light emitting device.

### PRIOR ART DOCUMENTS

### Patent Documents

(Patent Document 1) U.S. Pat. No. 4,356,429

### DISCLOSURE

### Technical Problem

An object of present disclosure is to provide a heterocyclic compound, an organic light emitting device comprising the same and a method for manufacturing the same.

### Technical Solution

In order to achieve the object, one embodiment of the present disclosure provides a heterocyclic compound represented by the following Chemical Formula 1.

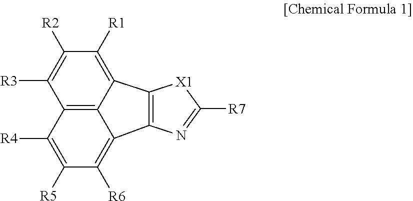

In Chemical Formula 1,
X1 is O; or S,
R1 to R6 are the same as or different from each other, and each independently selected from the group consisting of hydrogen; deuterium; halogen; a cyano group; a substituted or unsubstituted C1 to C60 alkyl group; a substituted or unsubstituted C2 to C60 alkenyl group; a substituted or unsubstituted C2 to C60 alkynyl group; a substituted or unsubstituted C1 to C60 alkoxy group; a substituted or unsubstituted C3 to C60 cycloalkyl group; a substituted or unsubstituted C2 to C60 heterocycloalkyl group; a substituted or unsubstituted C6 to C60 aryl group; a substituted or unsubstituted C2 to C60 heteroaryl group; —P(═O)R101R102; —SiR101R102R103; and a group represented by the following Chemical Formula 2, or two or more groups adjacent to each other bond to each other to form a substituted or unsubstituted C6 to C60 aromatic hydrocarbon ring or a substituted or unsubstituted C2 to C60 heteroring, wherein R101, R102 and R103 are the same as or different from each other and each independently a substituted or unsubstituted C1 to C60 alkyl group; a substituted or unsubstituted C6 to C60 aryl group; or a substituted or unsubstituted C2 to C60 heteroaryl group,
R7 is a substituted or unsubstituted C6 to C60 aryl group; or a group represented by the following Chemical Formula 2, and
at least one of R1 to R7 is the group represented by the following Chemical Formula 2,

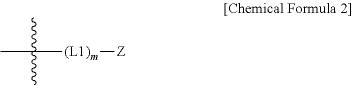

in Chemical Formula 2,
L1 is a direct bond; a substituted or unsubstituted C6 to C60 arylene group; or a substituted or unsubstituted C2 to C60 heteroarylene group,
m is an integer of 0 to 5, and when m is 2 or greater, L1s are the same as or different from each other, and
Z is selected from the group consisting of hydrogen; deuterium; halogen; a cyano group; a substituted or unsubstituted C1 to C60 alkyl group; a substituted or unsubstituted C2 to C60 alkenyl group; a substituted or unsubstituted C2 to C60 alkynyl group; a substituted or unsubstituted C1 to C60 alkoxy group; a substituted or unsubstituted C3 to C60 cycloalkyl group; a substituted or unsubstituted C2 to C60 heterocycloalkyl group; a substituted or unsubstituted C6 to C60 aryl group; a substituted or unsubstituted C2 to C60 heteroaryl group; a substituted or unsubstituted C6 to C60 monocyclic or polycyclic arylamine group; a substituted or unsubstituted C2 to C60 monocyclic or polycyclic heteroarylamine group; —P(═O)R101R102; and —SiR101R102R103, or two or more groups adjacent to each other bond to each other to form a substituted or unsubstituted C6 to C60 aromatic hydrocarbon ring or a substituted or unsubstituted C2 to C60 heteroring, wherein R101, R102 and R103 are the same as or different from each other and each independently a substituted or unsubstituted C1 to C60 alkyl group; a substituted or unsubstituted C6 to C60 aryl group; or a substituted or unsubstituted C2 to C60 heteroaryl group.

In addition, the present disclosure provides an organic light emitting device comprising:
a first electrode;
a second electrode provided opposite to the first electrode; and
one or more organic material layers provided between the first electrode and the second electrode,
wherein at least one of the one or more organic material layers comprise the heterocyclic compound represented by Chemical Formula 1.

### Advantageous Effects

A compound described in the present specification can be used as a material of an organic material layer of an organic light emitting device. The compound is capable of performing roles of a hole injection layer material, a hole transport layer material, a light emitting layer material, an electron transport layer material, an electron injection layer material and the like in an organic light emitting device. Particularly, the compound can be used as a light emitting layer material of an organic light emitting device, and the compound can be used as a light emitting material either alone or can be used as a host material or a dopant material of a light emitting layer.

Specifically, using the compound represented by Chemical Formula 1 as a host material of a light emitting layer is capable of lowering a driving voltage, enhancing light emission efficiency and enhancing lifetime properties in an organic light emitting device.

### DESCRIPTION OF DRAWINGS

FIGS. 1 to 4 are diagrams each schematically illustrating a lamination structure of an organic light emitting device according to one embodiment of the present disclosure.

### MODE FOR DISCLOSURE

Hereinafter, the present disclosure will be described in more detail.

In the present specification, a term “substitution” means that a hydrogen atom bonding to a carbon atom of a compound is changed to another substituent, and the position of substitution is not limited as long as it is a position at which the hydrogen atom is substituted, that is, a position at which a substituent is capable of substituting, and when two or more substituents substitute, the two or more substituents may be the same as or different from each other.

In the present specification, “substituted or unsubstituted” means being substituted with one or more substituents selected from the group consisting of deuterium; halogen; a cyano group; a C1 to C60 linear or branched alkyl group; a C2 to C60 linear or branched alkenyl group; a C2 to C60 linear or branched alkynyl group; a C3 to C60 monocyclic or polycyclic cycloalkyl group; a C2 to C60 monocyclic or polycyclic heterocycloalkyl group; a C6 to C60 monocyclic or polycyclic aryl group; a C2 to C60 monocyclic or polycyclic heteroaryl group; —SiRR′R″; —P(═O)RR′; a C1 to C20 alkylamine group; a C6 to C60 monocyclic or polycyclic arylamine group; and a C2 to C60 monocyclic or polycyclic heteroarylamine group or being unsubstituted, or being substituted with a substituent in which two or more substituents selected from among the substituents exemplified above are linked or being unsubstituted.

In the present specification, the halogen may be fluorine, chlorine, bromine or iodine.

In the present specification, the alkyl group includes linear or branched having 1 to 60 carbon atoms, and may be further substituted with other substituents. The number of carbon atoms of the alkyl group may be from 1 to 60, specifically from 1 to and more specifically from 1 to 20. Specific examples thereof may include a methyl group, an ethyl group, a propyl group, an n-propyl group, an isopropyl group, a butyl group, an n-butyl group, an isobutyl group, a tert-butyl group, a sec-butyl group, a 1-methyl-butyl group, a 1-ethyl-butyl group, a pentyl group, an n-pentyl group, an isopentyl group, a neopentyl group, a tert-pentyl group, a hexyl group, an n-hexyl group, a 1-methylpentyl group, a 2-methylpentyl group, a 4-methyl-2-pentyl group, a 3,3-dimethylbutyl group, a 2-ethylbutyl group, a heptyl group, an n-heptyl group, a 1-methylhexyl group, a cyclopentylmethyl group, a cyclohexylmethyl group, an octyl group, an n-octyl group, a tert-octyl group, a 1-methylheptyl group, a 2-ethylhexyl group, a 2-propylpentyl group, an n-nonyl group, a 2,2-dimethylheptyl group, a 1-ethyl-propyl group, a 1,1-dimethyl-propyl group, an isohexyl group, a 4-methylhexyl group, a 5-methylhexyl group and the like, but are not limited thereto.

In the present specification, the alkenyl group includes linear or branched having 2 to 60 carbon atoms, and may be further substituted with other substituents. The number of carbon atoms of the alkenyl group may be from 2 to 60, specifically from 2 to and more specifically from 2 to 20. Specific examples thereof may include a vinyl group, a 1-propenyl group, an isopropenyl group, a 1-butenyl group, a 2-butenyl group, a 3-butenyl group, a 1-pentenyl group, a 2-pentenyl group, a 3-pentenyl group, a 3-methyl-1-butenyl group, a 1,3-butadienyl group, an allyl group, a 1-phenylvinyl-1-yl group, a 2-phenylvinyl-1-yl group, a 2,2-diphenylvinyl-1-yl group, a 2-phenyl-2-(naphthyl-1-yl)vinyl-1-yl group, a 2,2-bis(diphenyl-1-yl)vinyl-1-yl group, a stilbenyl group, a styrenyl group and the like, but are not limited thereto.

In the present specification, the alkynyl group includes linear or branched having 2 to 60 carbon atoms, and may be further substituted with other substituents. The number of carbon atoms of the alkynyl group may be from 2 to 60, specifically from 2 to and more specifically from 2 to 20.

In the present specification, the alkoxy group may be linear, branched or cyclic. The number of carbon atoms of the alkoxy group is not particularly limited, but is preferably from 1 to 20. Specific examples thereof may include a methoxy group, an ethoxy group, an n-propoxy group, an isopropoxy group, an n-butoxy group, an isobutoxy group, a tert-butoxy group, a sec-butoxy group, an n-pentyloxy group, a neopentyloxy group, an isopentyloxy group, an n-hexyloxy group, a 3,3-dimethylbutyloxy group, a 2-ethylbutyloxy group, an n-octyloxy group, an n-nonyloxy group, an n-decyloxy group, a benzyloxy group, a p-methylbenzyloxy group and the like, but are not limited thereto.

In the present specification, the cycloalkyl group includes monocyclic or polycyclic having 3 to 60 carbon atoms, and may be further substituted with other substituents. Herein, the polycyclic means a group in which the cycloalkyl group is directly linked to or fused with other cyclic groups. Herein, the other cyclic groups may be a cycloalkyl group, but may also be different types of cyclic groups such as a heterocycloalkyl group, an aryl group and a heteroaryl group. The number of carbon atoms of the cycloalkyl group may be from 3 to 60, specifically from 3 to 40 and more specifically from 5 to 20. Specific examples thereof may include a cyclopropyl group, a cyclobutyl group, a cyclopentyl group, a 3-methylcyclopentyl group, a 2,3-dimethylcyclopentyl group, a cyclohexyl group, a 3-methylcyclohexyl group, a 4-methylcyclohexyl group, a 2,3-dimethylcyclohexyl group, a 3,4,5-trimethylcyclohexyl group, a 4-tert-butylcyclohexyl group, a cycloheptyl group, a cyclooctyl group and the like, but are not limited thereto.

In the present specification, the heterocycloalkyl group includes O, S, Se, N or Si as a heteroatom, includes monocyclic or polycyclic having 2 to 60 carbon atoms, and may be further substituted with other substituents. Herein, the polycyclic means a group in which the heterocycloalkyl group is directly linked to or fused with other cyclic groups. Herein, the other cyclic groups may be a heterocycloalkyl group, but may also be different types of cyclic groups such as a cycloalkyl group, an aryl group and a heteroaryl group. The number of carbon atoms of the heterocycloalkyl group may be from 2 to 60, specifically from 2 to 40 and more specifically from 3 to 20.

In the present specification, the aryl group includes monocyclic or polycyclic having 6 to 60 carbon atoms, and may be further substituted with other substituents. Herein, the polycyclic means a group in which the aryl group is directly linked to or fused with other cyclic groups. Herein, the other cyclic groups may be an aryl group, but may also be different types of cyclic groups such as a cycloalkyl group, a heterocycloalkyl group and a heteroaryl group. The aryl group may include a spiro group. The number of carbon atoms of the aryl group may be from 6 to 60, specifically from 6 to 40 and more specifically from 6 to 25. Specific examples of the aryl group may include a phenyl group, a biphenyl group, a triphenyl group, a naphthyl group, an anthryl group, a chrysenyl group, a phenanthrenyl group, a perylenyl group, a fluoranthenyl group, a triphenylenyl group, a phenalenyl group, a pyrenyl group, a tetracenyl group, a pentacenyl group, a fluorenyl group, an indenyl group, an acenaphthylenyl group, a benzofluorenyl group, a spirobifluorenyl group, a 2,3-dihydro-1H-indenyl group, a fused ring group thereof, and the like, but are not limited thereto.

In the present specification, the phosphine oxide group is represented by —P(═O)R101R102, wherein R101 and R102 are the same as or different from each other and may be each independently a substituent formed with at least one of hydrogen; deuterium; a halogen group; an alkyl group; an alkenyl group; an alkoxy group; a cycloalkyl group; an aryl group; and a heterocyclic group. Specifically, the phosphine oxide group may be substituted with an aryl group, and as the aryl group, the examples described above may be applied. Examples of the phosphine oxide group may include a diphenylphosphine oxide group, a dinaphthylphosphine oxide group and the like, but are not limited thereto.

In the present specification, the silyl group is a substituent including Si and having the Si atom directly linked as a radical, and is represented by —SiR101R102R103. R101 to R103 are the same as or different from each other, and may be each independently a substituent formed with at least one of hydrogen; deuterium; a halogen group; an alkyl group; an alkenyl group; an alkoxy group; a cycloalkyl group; an aryl group; and a heterocyclic group. Specific examples of the silyl group may include a trimethylsilyl group, a triethylsilyl group, a t-butyldimethylsilyl group, a vinyldimethylsilyl group, a propyldimethylsilyl group, a triphenylsilyl group, a diphenylsilyl group, a phenylsilyl group and the like, but are not limited thereto.

In the present specification, the fluorenyl group may be substituted, and adjacent substituents may bond to each other to form a ring.

When the fluorenyl group is substituted,

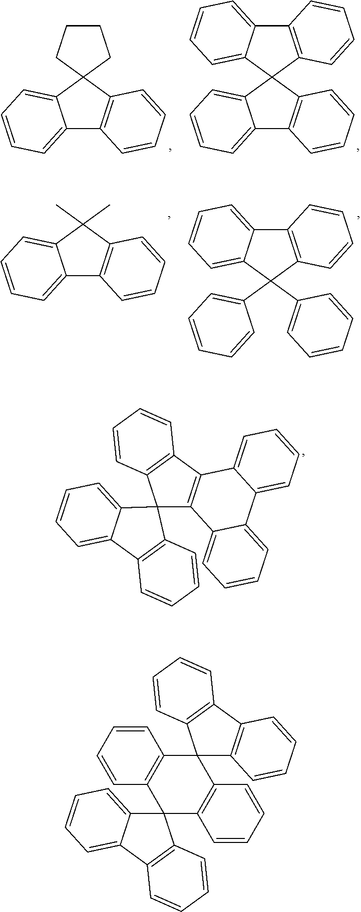

and the like may be included, however, the structure is not limited thereto.

In the present specification, the spiro group is a group including a spiro structure, and may have 15 to 60 carbon atoms. For example, the spiro group may include a structure in which a 2,3-dihydro-1H-indene group or a cyclohexane group spiro bonds to a fluorenyl group. Specifically, the spiro group may include any one of groups of the following structural formulae.

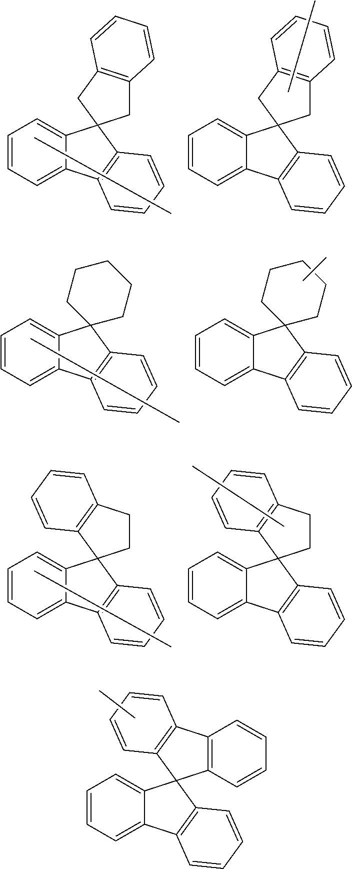

In the present specification, the heteroaryl group includes S, O, Se, N or Si as a heteroatom, includes monocyclic or polycyclic having 2 to 60 carbon atoms, and may be further substituted with other substituents. Herein, the polycyclic means a group in which the heteroaryl group is directly linked to or fused with other cyclic groups. Herein, the other cyclic groups may be a heteroaryl group, but may also be different types of cyclic groups such as a cycloalkyl group, a heterocycloalkyl group and an aryl group. The number of carbon atoms of the heteroaryl group may be from 2 to 60, specifically from 2 to 40 and more specifically from 3 to 25. Specific examples of the heteroaryl group may include a pyridyl group, a pyrrolyl group, a pyrimidyl group, a pyridazinyl group, a furanyl group, a thiophenyl group, an imidazolyl group, a pyrazolyl group, an oxazolyl group, an isoxazolyl group, a thiazolyl group, an isothiazolyl group, a triazolyl group, a furazanyl group, an oxadiazolyl group, a thiadiazolyl group, a dithiazolyl group, a tetrazolyl group, a pyranyl group, a thiopyranyl group, a diazinyl group, an oxazinyl group, a thiazinyl group, a dioxynyl group, a triazinyl group, a tetrazinyl group, a quinolyl group, an isoquinolyl group, a quinazolinyl group, an isoquinazolinyl group, a qninozolinyl group, a naphthyridyl group, an acridinyl group, a phenanthridinyl group, an imidazopyridinyl group, a diazanaphthalenyl group, a triazaindenyl group, a 2-indolyl group, an indolizinyl group, a benzothiazolyl group, a benzoxazolyl group, a benzimidazolyl group, a benzothiophenyl group, a benzofuranyl group, a dibenzothiophenyl group, a dibenzofuranyl group, a carbazolyl group, a benzocarbazolyl group, a dibenzocarbazolyl group, a phenazinyl group, a dibenzosilole group, a spirobi(dibenzosilole) group, a dihydrophenazinyl group, a phenoxazinyl group, a phenanthridyl group, a thienyl group, an indolo[2,3-a]carbazolyl group, an indolo[2,3-b]carbazolyl group, an indolinyl group, a 10,11-dihydro-dibenzo[b, f]azepinyl group, a 9,10-dihydroacridinyl group, a phenanthrazinyl group, a phenothiazinyl group, a phthalazinyl group, a naphthylidinyl group, a phenanthrolinyl group, a benzo[c][1,2,5]thiadiazolyl group, a 5,10-dihydrodibenzo[b, e][1, 4]azasilinyl group, a pyrazolo[1,5-c]quinazolinyl group, a pyrido[1,2-b]indazolyl group, a pyrido[1,2-a]imidazo[1,2-e]indolinyl group, a 5,11-dihydroindeno[1,2-b]carbazolyl group and the like, but are not limited thereto.

In the present specification, the amine group may be selected from the group consisting of a monoalkylamine group; a monoarylamine group; a monoheteroarylamine group; —NH₂; a dialkylamine group; a diarylamine group; a diheteroarylamine group; an alkylarylamine group; an alkylheteroarylamine group; and an arylheteroarylamine group, and the number of carbon atoms is not particularly limited, but preferably from 1 to 30. Specific examples of the amine group may include a methylamine group, a dimethylamine group, an ethylamine group, a diethylamine group, a phenylamine group, a naphthylamine group, a biphenylamine group, a dibiphenylamine group, an anthracenylamine group, a 9-methyl-anthracenylamine group, a diphenylamine group, a phenylnaphthylamine group, a ditolylamine group, a phenyltolylamine group, a triphenylamine group, a biphenylnaphthylamine group, a phenylbiphenylamine group, a biphenylfluorenylamine group, a phenyltriphenylenylamine group, a biphenyltriphenylenylamine group and the like, but are not limited thereto.

In the present specification, the arylene group means the aryl group having two bonding sites, that is, a divalent group. The descriptions on the aryl group provided above may be applied thereto except for those that are each a divalent group. In addition, the heteroarylene group means the heteroaryl group having two bonding sites, that is, a divalent group. The descriptions on the heteroaryl group provided above may be applied thereto except for those that are each a divalent group.

In the present specification, an “adjacent” group may mean a substituent substituting an atom directly linked to an atom substituted by the corresponding substituent, a substituent sterically most closely positioned to the corresponding substituent, or another substituent substituting an atom substituted by the corresponding substituent. For example, two substituents substituting at ortho positions in a benzene ring, and two substituents substituting at the same carbon in an aliphatic ring may be interpreted as groups “adjacent” to each other.

In the present disclosure, a “case of a substituent being not indicated in a chemical formula or compound structure” means that a hydrogen atom bonds to a carbon atom. However, since deuterium (²H) is an isotope of hydrogen, some hydrogen atoms may be deuterium.

In one embodiment of the present disclosure, a “case of a substituent being not indicated in a chemical formula or compound structure” may mean that positions that may come as a substituent are all hydrogen or deuterium. In other words, since deuterium is an isotope of hydrogen, some hydrogen atoms may be deuterium that is an isotope, and herein, a content of the deuterium may be from 0% to 100%.

In one embodiment of the present disclosure, in a “case of a substituent being not indicated in a chemical formula or compound structure”, hydrogen and deuterium may be used interchangeably in compounds when deuterium is not explicitly excluded such as “a deuterium content being 0%”, “a hydrogen content being 100%” or “substituents being all hydrogen”.

In one embodiment of the present disclosure, deuterium is one of isotopes of hydrogen, is an element having deuteron formed with one proton and one neutron as a nucleus, and may be expressed as hydrogen-2, and the elemental symbol thereof may also be written as D or ²H.

In one embodiment of the present disclosure, an isotope means an atom with the same atomic number (Z) but with a different mass number (A), and may also be interpreted as an element with the same number of protons but with a different number of neutrons.

In one embodiment of the present disclosure, a meaning of a content T % of a specific substituent may be defined as T2/T1×100=T % when the total number of substituents that a basic compound may have is defined as T1, and the number of specific substituents among these is defined as T2.

In other words, in one example, having a deuterium content of 20% in a phenyl group represented by

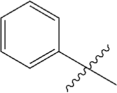 [SMILES: *c1ccccc1]

may mean that the total number of substituents that the phenyl group may have is 5 (T1 in the formula), and the number of deuterium among these is 1 (T2 in the formula). In other words, having a deuterium content of 20% in a phenyl group may be represented by the following structural formulae.

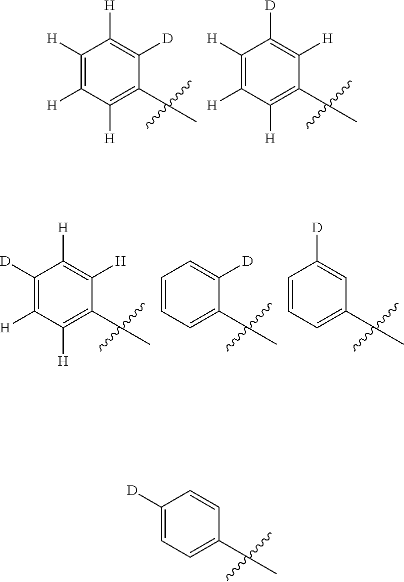

In addition, in one embodiment of the present disclosure, “a phenyl group having a deuterium content of 0%” may mean a phenyl group that does not include a deuterium atom, that is, a phenyl group that has 5 hydrogen atoms.

In the present disclosure, a heterocyclic compound represented by Chemical Formula 1 may have a deuterium content of 0% to 100%, and more preferably 30% to 100%.

In the present disclosure, the C6 to C60 aromatic hydrocarbon ring means a compound including an aromatic ring formed with C6 to C60 carbons and hydrogens. Examples thereof may include phenyl, biphenyl, terphenyl, triphenylene, naphthalene, anthracene, phenalene, phenanthrene, fluorene, pyrene, chrysene, perylene, azulene and the like, but are not limited thereto, and include all aromatic hydrocarbon ring compounds known in the art and satisfying the above-mentioned number of carbon atoms.

In the present specification, an electron transporting group means a functional group having greater electron transporting properties than hole transporting properties, and may be referred to as an N-type functional group.

In the present specification, a hole transporting group means a functional group having greater hole transporting properties than electron transporting properties, and may be referred to as a P-type functional group. Examples thereof may include carbazoles, indolocarbazoles, indolothiophenes, indolodibenzofurans, biscarbazoles, arylamine groups and the like, but are not limited thereto.

One embodiment of the present disclosure provides a heterocyclic compound represented by the following Chemical Formula 1.

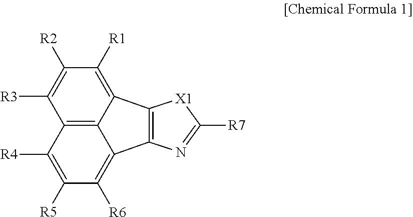

In Chemical Formula 1,
X1 is O; or S,
R1 to R6 are the same as or different from each other, and each independently selected from the group consisting of hydrogen; deuterium; halogen; a cyano group; a substituted or unsubstituted C1 to C60 alkyl group; a substituted or unsubstituted C2 to C60 alkenyl group; a substituted or unsubstituted C2 to C60 alkynyl group; a substituted or unsubstituted C1 to C60 alkoxy group; a substituted or unsubstituted C3 to C60 cycloalkyl group; a substituted or unsubstituted C2 to C60 heterocycloalkyl group; a substituted or unsubstituted C6 to C60 aryl group; a substituted or unsubstituted C2 to C60 heteroaryl group; —P(═O)R101R102; —SiR101R102R103; and a group represented by the following Chemical Formula 2, or two or more groups adjacent to each other bond to each other to form a substituted or unsubstituted C6 to C60 aromatic hydrocarbon ring or a substituted or unsubstituted C2 to C60 heteroring, wherein R101, R102 and R103 are the same as or different from each other and each independently a substituted or unsubstituted C1 to C60 alkyl group; a substituted or unsubstituted C6 to C60 aryl group; or a substituted or unsubstituted C2 to C60 heteroaryl group,
R7 is a substituted or unsubstituted C6 to C60 aryl group; or a group represented by the following Chemical Formula 2, and
at least one of R1 to R7 is the group represented by the following Chemical Formula 2,

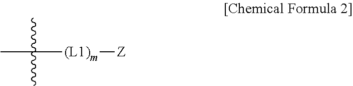

in Chemical Formula 2,
L1 is a direct bond; a substituted or unsubstituted C6 to C60 arylene group; or a substituted or unsubstituted C2 to C60 heteroarylene group,
m is an integer of 0 to 5, and when m is 2 or greater, L1s are the same as or different from each other, and
Z is selected from the group consisting of hydrogen; deuterium; halogen; a cyano group; a substituted or unsubstituted C1 to C60 alkyl group; a substituted or unsubstituted C2 to C60 alkenyl group; a substituted or unsubstituted C2 to C60 alkynyl group; a substituted or unsubstituted C1 to C60 alkoxy group; a substituted or unsubstituted C3 to C60 cycloalkyl group; a substituted or unsubstituted C2 to C60 heterocycloalkyl group; a substituted or unsubstituted C6 to C60 aryl group; a substituted or unsubstituted C2 to C60 heteroaryl group; a substituted or unsubstituted C6 to C60 monocyclic or polycyclic arylamine group; a substituted or unsubstituted C2 to C60 monocyclic or polycyclic heteroarylamine group; —P(═O)R101R102; and —SiR101R102R103, or two or more groups adjacent to each other bond to each other to form a substituted or unsubstituted C6 to C60 aromatic hydrocarbon ring or a substituted or unsubstituted C2 to C60 heteroring, wherein R101, R102 and R103 are the same as or different from each other and each independently a substituted or unsubstituted C1 to C60 alkyl group; a substituted or unsubstituted C6 to C60 aryl group; or a substituted or unsubstituted C2 to C60 heteroaryl group.

In one embodiment of the present disclosure, R1 to R6 are the same as or different from each other, and each independently hydrogen; deuterium; halogen; a cyano group; a substituted or unsubstituted C1 to C30 alkyl group; a substituted or unsubstituted C2 to C30 alkenyl group; a substituted or unsubstituted C2 to C30 alkynyl group; a substituted or unsubstituted C1 to C30 alkoxy group; a substituted or unsubstituted C3 to C30 cycloalkyl group; a substituted or unsubstituted C2 to C30 heterocycloalkyl group; a substituted or unsubstituted C6 to C30 aryl group; a substituted or unsubstituted C2 to C30 heteroaryl group; —P(═O)R101R102; —SiR101R102R103; or the group represented by Chemical Formula 2, or two or more groups adjacent to each other bond to each other to form a substituted or unsubstituted C6 to C30 aromatic hydrocarbon ring; or a substituted or unsubstituted C2 to C30 heteroring, wherein R101, R102 and R103 are the same as or different from each other and may be each independently a substituted or unsubstituted C1 to C30 alkyl group; a substituted or unsubstituted C6 to C30 aryl group; or a substituted or unsubstituted C2 to C30 heteroaryl group.

In another embodiment of the present disclosure, R1 to R6 are the same as or different from each other, and each independently hydrogen; deuterium; halogen; a cyano group; a substituted or unsubstituted C1 to C20 alkyl group; a substituted or unsubstituted C2 to C20 alkenyl group; a substituted or unsubstituted C2 to C20 alkynyl group; a substituted or unsubstituted C1 to C20 alkoxy group; a substituted or unsubstituted C3 to C20 cycloalkyl group; a substituted or unsubstituted C2 to C20 heterocycloalkyl group; a substituted or unsubstituted C6 to C20 aryl group; a substituted or unsubstituted C2 to C20 heteroaryl group; —P(═O)R101R102; —SiR101R102R103; or the group represented by Chemical Formula 2, or two or more groups adjacent to each other bond to each other to form a substituted or unsubstituted C6 to C20 aromatic hydrocarbon ring; or a substituted or unsubstituted C2 to C20 heteroring, wherein R101, R102 and R103 are the same as or different from each other and may be each independently a substituted or unsubstituted C1 to C20 alkyl group; a substituted or unsubstituted C6 to C20 aryl group; or a substituted or unsubstituted C2 to C20 heteroaryl group.

In another embodiment of the present disclosure, R1 to R6 are the same as or different from each other, and each independently hydrogen; deuterium; a substituted or unsubstituted C1 to C20 alkyl group; a substituted or unsubstituted C6 to C20 aryl group; a substituted or unsubstituted C2 to C20 heteroaryl group; or the group represented by Chemical Formula 2, or two or more groups adjacent to each other may bond to each other to form a substituted or unsubstituted C6 to C20 aromatic hydrocarbon ring; or a substituted or unsubstituted C2 to C20 heteroring.

In another embodiment of the present disclosure, R1 to R6 are the same as or different from each other, and each independently hydrogen; deuterium; or the group represented by Chemical Formula 2, or two or more groups adjacent to each other may bond to each other to form a substituted or unsubstituted C6 to C20 aromatic hydrocarbon ring; or a substituted or unsubstituted C2 to C20 heteroring.

In one embodiment of the present disclosure, R7 may be a substituted or unsubstituted C6 to C60 aryl group; or the group represented by Chemical Formula 2.

In another embodiment of the present disclosure, R7 may be a substituted or unsubstituted C6 to C30 aryl group; or the group represented by Chemical Formula 2.

In another embodiment of the present disclosure, R7 may be a substituted or unsubstituted C6 to C20 aryl group; or the group represented by Chemical Formula 2.

In another embodiment of the present disclosure, R7 may be a substituted or unsubstituted phenyl group; a substituted or unsubstituted biphenyl group; a substituted or unsubstituted naphthyl group; a substituted or unsubstituted phenanthrenyl group; a substituted or unsubstituted pyrenyl group; or the group represented by Chemical Formula 2.

In one embodiment of the present disclosure, L1 may be a direct bond; a substituted or unsubstituted C6 to C30 arylene group; or a substituted or unsubstituted C2 to C30 heteroarylene group.

In another embodiment of the present disclosure, L1 may be a direct bond; a substituted or unsubstituted C6 to C20 arylene group; or a substituted or unsubstituted C2 to C20 heteroarylene group.

In another embodiment of the present disclosure, L1 may be a direct bond; a substituted or unsubstituted phenylene group; a substituted or unsubstituted biphenylene group; a substituted or unsubstituted carbazolene group; or a substituted or unsubstituted benzocarbazolene group.

In one embodiment of the present disclosure, Z is a substituted or unsubstituted C2 to C60 heteroaryl group; a substituted or unsubstituted C6 to C60 monocyclic or polycyclic arylamine group; or a substituted or unsubstituted C2 to C60 monocyclic or polycyclic heteroarylamine group, or two or more groups adjacent to each other may bond to each other to form a substituted or unsubstituted C6 to C60 aromatic hydrocarbon ring or a substituted or unsubstituted C2 to C60 heteroring.

In another embodiment of the present disclosure, Z is hydrogen; deuterium; halogen; a cyano group; a substituted or unsubstituted C1 to C30 alkyl group; a substituted or unsubstituted C2 to C30 alkenyl group; a substituted or unsubstituted C2 to C30 alkynyl group; a substituted or unsubstituted C1 to C30 alkoxy group; a substituted or unsubstituted C3 to C30 cycloalkyl group; a substituted or unsubstituted C2 to C30 heterocycloalkyl group; a substituted or unsubstituted C6 to C30 aryl group; a substituted or unsubstituted C2 to C30 heteroaryl group; a substituted or unsubstituted C6 to C30 monocyclic or polycyclic arylamine group; a substituted or unsubstituted C2 to C30 monocyclic or polycyclic heteroarylamine group; —P(═O)R101R102; or —SiR101R102R103, or two or more groups adjacent to each other bond to each other to form a substituted or unsubstituted C6 to C30 aromatic hydrocarbon ring or a substituted or unsubstituted C2 to C30 heteroring, wherein R101, R102 and R103 are the same as or different from each other and may be each independently a substituted or unsubstituted C1 to C30 alkyl group; a substituted or unsubstituted C6 to C30 aryl group; or a substituted or unsubstituted C2 to C30 heteroaryl group.

In another embodiment of the present disclosure, Z is hydrogen; deuterium; halogen; a cyano group; a substituted or unsubstituted C1 to C20 alkyl group; a substituted or unsubstituted C2 to C20 alkenyl group; a substituted or unsubstituted C2 to C20 alkynyl group; a substituted or unsubstituted C1 to C20 alkoxy group; a substituted or unsubstituted C3 to C20 cycloalkyl group; a substituted or unsubstituted C2 to C20 heterocycloalkyl group; a substituted or unsubstituted C6 to C20 aryl group; a substituted or unsubstituted C2 to C20 heteroaryl group; a substituted or unsubstituted C6 to C20 monocyclic or polycyclic arylamine group; a substituted or unsubstituted C2 to C20 monocyclic or polycyclic heteroarylamine group; —P(═O)R101R102; or —SiR101R102R103, or two or more groups adjacent to each other bond to each other to form a substituted or unsubstituted C6 to C20 aromatic hydrocarbon ring or a substituted or unsubstituted C2 to C20 heteroring, wherein R101, R102 and R103 are the same as or different from each other and may be each independently a substituted or unsubstituted C1 to C20 alkyl group; a substituted or unsubstituted C6 to C20 aryl group; or a substituted or unsubstituted C2 to C20 heteroaryl group.

In another embodiment of the present disclosure, Z is a substituted or unsubstituted C2 to C20 heteroaryl group; a substituted or unsubstituted C6 to C20 monocyclic or polycyclic arylamine group; or a substituted or unsubstituted C2 to C20 monocyclic or polycyclic heteroarylamine group, or two or more groups adjacent to each other may bond to each other to form a substituted or unsubstituted C6 to C20 aromatic hydrocarbon ring or a substituted or unsubstituted C2 to C20 heteroring.

In one embodiment of the present disclosure, the heterocyclic compound represented by Chemical Formula 1 may not include deuterium as a substituent, or may have a deuterium content of greater than 0%, 1% or greater, 10% or greater, 20% or greater, 30% or greater, 40% or greater or 50% or greater, and 100% or less, 90% or less, 80% or less, 70% or less or 60% or less with respect to the total number of hydrogen atoms and deuterium atoms.

In another embodiment of the present disclosure, the heterocyclic compound represented by Chemical Formula 1 may not include deuterium as a substituent, or may have a deuterium content of 1% to 100% based on the total number of hydrogen atoms and deuterium atoms.

In another embodiment of the present disclosure, the heterocyclic compound represented by Chemical Formula 1 may not include deuterium as a substituent, or may have a deuterium content of 20% to 90% based on the total number of hydrogen atoms and deuterium atoms.

In another embodiment of the present disclosure, the heterocyclic compound represented by Chemical Formula 1 may not include deuterium as a substituent, or may have a deuterium content of 30% to 80% based on the total number of hydrogen atoms and deuterium atoms.

In another embodiment of the present disclosure, the heterocyclic compound represented by Chemical Formula 1 may not include deuterium as a substituent, or may have a deuterium content of 50% to 70% based on the total number of hydrogen atoms and deuterium atoms.

In one embodiment of the present disclosure, Chemical Formula 1 may be a heterocyclic compound represented by any one of the following Chemical Formula 1-1 or 1-2.

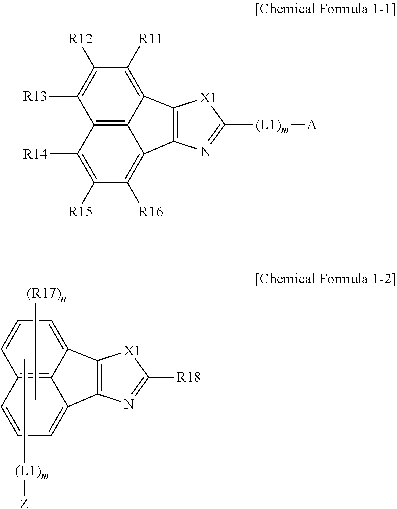

In Chemical Formulae 1-1 and 1-2,
R11 to R17 are the same as or different from each other and each independently selected from the group consisting of hydrogen; deuterium; halogen; a cyano group; a substituted or unsubstituted C1 to C60 alkyl group; a substituted or unsubstituted C2 to C60 alkenyl group; a substituted or unsubstituted C2 to C60 alkynyl group; a substituted or unsubstituted C1 to C60 alkoxy group; a substituted or unsubstituted C3 to C60 cycloalkyl group; a substituted or unsubstituted C2 to C60 heterocycloalkyl group; a substituted or unsubstituted C6 to C60 aryl group; a substituted or unsubstituted C2 to C60 heteroaryl group; —P(═O)R101R102; and —SiR101R102R103, wherein R101, R102 and R103 are the same as or different from each other and each independently a substituted or unsubstituted C1 to C60 alkyl group; a substituted or unsubstituted C6 to C60 aryl group; or a substituted or unsubstituted C2 to C60 heteroaryl group,
R18 is a substituted or unsubstituted C6 to C60 aryl group,
n is an integer of 0 to 5, and when n is 2 or greater, R17s are the same as or different from each other,
X1 has the same definition as in Chemical Formula 1, and
L1, m and Z have the same definitions as in Chemical Formula 2.

In one embodiment of the present disclosure, R11 to R17 are the same as or different from each other and each independently hydrogen; deuterium; halogen; a cyano group; a substituted or unsubstituted C1 to C30 alkyl group; a substituted or unsubstituted C2 to C30 alkenyl group; a substituted or unsubstituted C2 to C30 alkynyl group; a substituted or unsubstituted C1 to C30 alkoxy group; a substituted or unsubstituted C3 to C30 cycloalkyl group; a substituted or unsubstituted C2 to C30 heterocycloalkyl group; a substituted or unsubstituted C6 to C30 aryl group; a substituted or unsubstituted C2 to C30 heteroaryl group; —P(═O)R101R102; or —SiR101R102R103, wherein R101, R102 and R103 are the same as or different from each other and may be each independently a substituted or unsubstituted C1 to C30 alkyl group; a substituted or unsubstituted C6 to C30 aryl group; or a substituted or unsubstituted C2 to C30 heteroaryl group.

In another embodiment of the present disclosure, R11 to R17 are the same as or different from each other and each independently hydrogen; deuterium; halogen; a cyano group; a substituted or unsubstituted C1 to C20 alkyl group; a substituted or unsubstituted C2 to C20 alkenyl group; a substituted or unsubstituted C2 to C20 alkynyl group; a substituted or unsubstituted C1 to C20 alkoxy group; a substituted or unsubstituted C3 to C20 cycloalkyl group; a substituted or unsubstituted C2 to C20 heterocycloalkyl group; a substituted or unsubstituted C6 to C20 aryl group; a substituted or unsubstituted C2 to C20 heteroaryl group; —P(═O)R101R102; or —SiR101R102R103, wherein R101, R102 and R103 are the same as or different from each other and may be each independently a substituted or unsubstituted C1 to C20 alkyl group; a substituted or unsubstituted C6 to C20 aryl group; or a substituted or unsubstituted C2 to C20 heteroaryl group.

In another embodiment of the present disclosure, R11 to R17 are the same as or different from each other, and may be each independently hydrogen; deuterium; a substituted or unsubstituted C1 to C20 alkyl group; a substituted or unsubstituted C6 to C20 aryl group; or a substituted or unsubstituted C2 to C20 heteroaryl group.

In another embodiment of the present disclosure, R11 to R17 are the same as or different from each other, and may be each independently hydrogen; or deuterium.

In one embodiment of the present disclosure, R18 may be a substituted or unsubstituted C6 to C30 aryl group.

In another embodiment of the present disclosure, R18 may be a substituted or unsubstituted C6 to C20 aryl group.

In another embodiment of the present disclosure, R18 may be a substituted or unsubstituted phenyl group; a substituted or unsubstituted biphenyl group; a substituted or unsubstituted naphthyl group; a substituted or unsubstituted phenanthrenyl group; or a substituted or unsubstituted pyrenyl group.

In one embodiment of the present disclosure, Chemical Formula 1-2 may be represented by any one of the following Chemical Formulae 1-2-1 to 1-2-4.

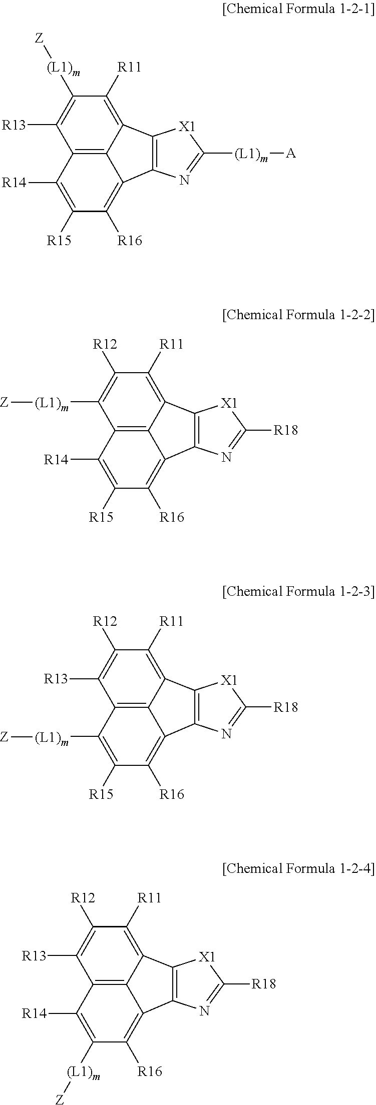

In Chemical Formulae 1-2-1 to 1-2-4,
X1 has the same definition as in Chemical Formula 1,
R11 to R16 have the same definitions as in Chemical Formula 1-1,
R18 has the same definition as in Chemical Formula 1-2, and
L1, m and Z have the same definitions as in Chemical Formula 2.

In one embodiment of the present disclosure, Chemical Formula 1 may be represented by any one of the following Chemical Formulae 1-3-1 to 1-3-3.

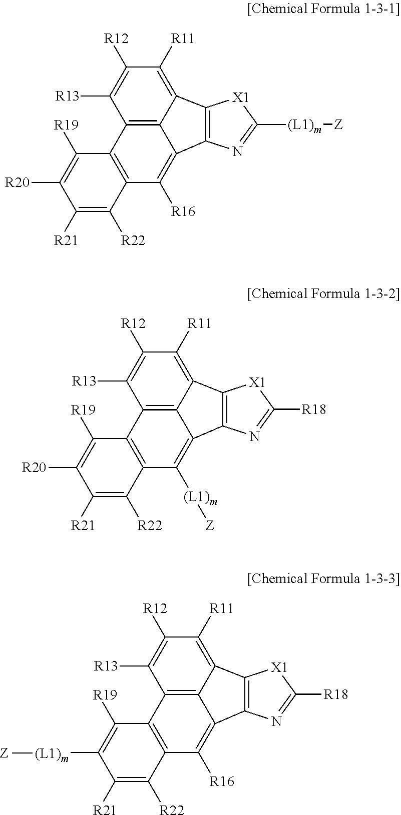

In Chemical Formulae 1-3-1 to 1-3-3,
R19 to R22 are the same as or different from each other and each independently selected from the group consisting of hydrogen; deuterium; halogen; a cyano group; a substituted or unsubstituted C1 to C60 alkyl group; a substituted or unsubstituted C2 to C60 alkenyl group; a substituted or unsubstituted C2 to C60 alkynyl group; a substituted or unsubstituted C1 to C60 alkoxy group; a substituted or unsubstituted C3 to C60 cycloalkyl group; a substituted or unsubstituted C2 to C60 heterocycloalkyl group; a substituted or unsubstituted C6 to C60 aryl group; a substituted or unsubstituted C2 to C60 heteroaryl group; —P(═O)R101R102; and —SiR101R102R103, wherein R101, R102 and R103 are the same as or different from each other and each independently a substituted or unsubstituted C1 to C60 alkyl group; a substituted or unsubstituted C6 to C60 aryl group; or a substituted or unsubstituted C2 to C60 heteroaryl group,
X1 has the same definition as in Chemical Formula 1,
R11 to R13 and R16 have the same definitions as in Chemical Formula 1-1,
R18 has the same definition as in Chemical Formula 1-2, and
L1, m and Z have the same definitions as in Chemical Formula 2.

In one embodiment of the present disclosure, R19 to R22 are the same as or different from each other and each independently hydrogen; deuterium; halogen; a cyano group; a substituted or unsubstituted C1 to C30 alkyl group; a substituted or unsubstituted C2 to C30 alkenyl group; a substituted or unsubstituted C2 to C30 alkynyl group; a substituted or unsubstituted C1 to C30 alkoxy group; a substituted or unsubstituted C3 to C30 cycloalkyl group; a substituted or unsubstituted C2 to C30 heterocycloalkyl group; a substituted or unsubstituted C6 to C30 aryl group; a substituted or unsubstituted C2 to C30 heteroaryl group; —P(═O)R101R102; or —SiR101R102R103, wherein R101, R102 and R103 are the same as or different from each other and may be each independently a substituted or unsubstituted C1 to C30 alkyl group; a substituted or unsubstituted C6 to C30 aryl group; or a substituted or unsubstituted C2 to C30 heteroaryl group.

In another embodiment of the present disclosure, R19 to R22 are the same as or different from each other and each independently hydrogen; deuterium; halogen; a cyano group; a substituted or unsubstituted C1 to C20 alkyl group; a substituted or unsubstituted C2 to C20 alkenyl group; a substituted or unsubstituted C2 to C20 alkynyl group; a substituted or unsubstituted C1 to C20 alkoxy group; a substituted or unsubstituted C3 to C20 cycloalkyl group; a substituted or unsubstituted C2 to C20 heterocycloalkyl group; a substituted or unsubstituted C6 to C20 aryl group; a substituted or unsubstituted C2 to C20 heteroaryl group; —P(═O)R101R102; or —SiR101R102R103, wherein R101, R102 and R103 are the same as or different from each other and may be each independently a substituted or unsubstituted C1 to C20 alkyl group; a substituted or unsubstituted C6 to C20 aryl group; or a substituted or unsubstituted C2 to C20 heteroaryl group.

In another embodiment of the present disclosure, R19 to R22 are the same as or different from each other, and may be each independently hydrogen; deuterium; a substituted or unsubstituted C1 to C20 alkyl group; a substituted or unsubstituted C6 to C20 aryl group; or a substituted or unsubstituted C2 to C20 heteroaryl group.

In another embodiment of the present disclosure, R19 to R22 are the same as or different from each other, and may be each independently hydrogen; or deuterium.

In one embodiment of the present disclosure, Chemical Formula 1 may be represented by any one of the following Chemical Formulae 1-4-1 and 1-4-2.

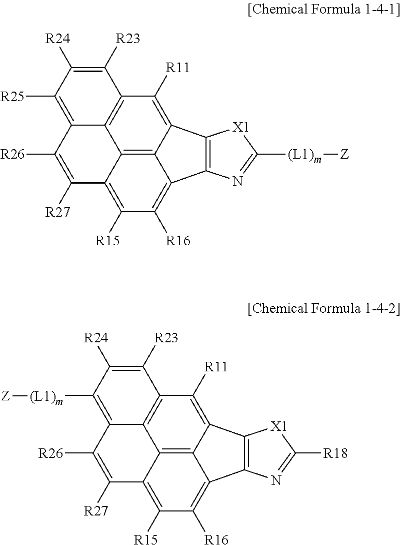

In Chemical Formulae 1-4-1 and 1-4-2,
R23 to R27 are the same as or different from each other and each independently selected from the group consisting of hydrogen; deuterium; halogen; a cyano group; a substituted or unsubstituted C1 to C60 alkyl group; a substituted or unsubstituted C2 to C60 alkenyl group; a substituted or unsubstituted C2 to C60 alkynyl group; a substituted or unsubstituted C1 to C60 alkoxy group; a substituted or unsubstituted C3 to C60 cycloalkyl group; a substituted or unsubstituted C2 to C60 heterocycloalkyl group; a substituted or unsubstituted C6 to C60 aryl group; a substituted or unsubstituted C2 to C60 heteroaryl group; —P(═O)R101R102; and —SiR101R102R103, wherein R101, R102 and R103 are the same as or different from each other and each independently a substituted or unsubstituted C1 to C60 alkyl group; a substituted or unsubstituted C6 to C60 aryl group; or a substituted or unsubstituted C2 to C60 heteroaryl group,
X1 has the same definition as in Chemical Formula 1,
R11, R15 and R16 have the same definitions as in Chemical Formula 1-1,
R18 has the same definition as in Chemical Formula 1-2, and
L1, m and Z have the same definitions as in Chemical Formula 2.

In one embodiment of the present disclosure, R23 to R27 are the same as or different from each other and each independently hydrogen; deuterium; halogen; a cyano group; a substituted or unsubstituted C1 to C30 alkyl group; a substituted or unsubstituted C2 to C30 alkenyl group; a substituted or unsubstituted C2 to C30 alkynyl group; a substituted or unsubstituted C1 to C30 alkoxy group; a substituted or unsubstituted C3 to C30 cycloalkyl group; a substituted or unsubstituted C2 to C30 heterocycloalkyl group; a substituted or unsubstituted C6 to C30 aryl group; a substituted or unsubstituted C2 to C30 heteroaryl group; —P(═O)R101R102; or —SiR101R102R103, wherein R101, R102 and R103 are the same as or different from each other and may be each independently a substituted or unsubstituted C1 to C30 alkyl group; a substituted or unsubstituted C6 to C30 aryl group; or a substituted or unsubstituted C2 to C30 heteroaryl group.

In another embodiment of the present disclosure, R23 to R27 are the same as or different from each other and each independently hydrogen; deuterium; halogen; a cyano group; a substituted or unsubstituted C1 to C20 alkyl group; a substituted or unsubstituted C2 to C20 alkenyl group; a substituted or unsubstituted C2 to C20 alkynyl group; a substituted or unsubstituted C1 to C20 alkoxy group; a substituted or unsubstituted C3 to C20 cycloalkyl group; a substituted or unsubstituted C2 to C20 heterocycloalkyl group; a substituted or unsubstituted C6 to C20 aryl group; a substituted or unsubstituted C2 to C20 heteroaryl group; —P(═O)R101R102; or —SiR101R102R103, wherein R101, R102 and R103 are the same as or different from each other and may be each independently a substituted or unsubstituted C1 to C20 alkyl group; a substituted or unsubstituted C6 to C20 aryl group; or a substituted or unsubstituted C2 to C20 heteroaryl group.

In another embodiment of the present disclosure, R23 to R27 are the same as or different from each other, and may be each independently hydrogen; deuterium; a substituted or unsubstituted C1 to C20 alkyl group; a substituted or unsubstituted C6 to C20 aryl group; or a substituted or unsubstituted C2 to C20 heteroaryl group.

In another embodiment of the present disclosure, R23 to R27 are the same as or different from each other, and may be each independently hydrogen; or deuterium.

In one embodiment of the present disclosure, Z may be a hole transporting group.

In one embodiment of the present disclosure, Z may be represented by any one of the following Chemical Formulae 3-1 and 3-2.

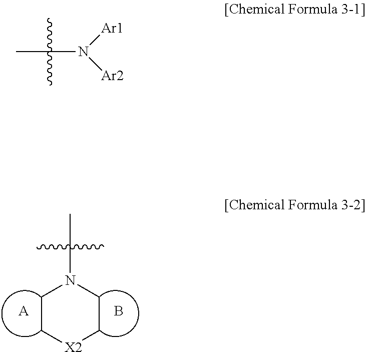

In Chemical Formulae 3-1 and 3-2,
Ar1 and Ar2 are the same as or different from each other, and each independently a substituted or unsubstituted C6 to C60 aryl group; or a substituted or unsubstituted C2 to C60 heteroaryl group,
X2 is a direct bond; 0; S; or NRa,
Ra is selected from the group consisting of hydrogen; deuterium; halogen; a cyano group; a substituted or unsubstituted C1 to C60 alkyl group; a substituted or unsubstituted C2 to C60 alkenyl group; a substituted or unsubstituted C2 to C60 alkynyl group; a substituted or unsubstituted C1 to C60 alkoxy group; a substituted or unsubstituted C3 to C60 cycloalkyl group; a substituted or unsubstituted C2 to C60 heterocycloalkyl group; a substituted or unsubstituted C6 to C60 aryl group; a substituted or unsubstituted C2 to C60 heteroaryl group; —P(═O)R101R102; and —SiR101R102R103, wherein R101, R102 and R103 are the same as or different from each other and each independently a substituted or unsubstituted C1 to C60 alkyl group; a substituted or unsubstituted C6 to C60 aryl group; or a substituted or unsubstituted C2 to C60 heteroaryl group, and
the A ring and the B ring are the same as or different from each other and each independently a substituted or unsubstituted C6 to C60 aryl ring; a substituted or unsubstituted C4 to C60 heteroaryl ring; a substituted or unsubstituted C5 to C60 cycloalkyl ring; or a substituted or unsubstituted C5 to C60 cycloalkenyl ring, two or more groups of substituents of the A ring and the B ring adjacent to each other may bond to each other to form a substituted or unsubstituted C5 to C60 hydrocarbon ring or a substituted or unsubstituted C4 to C60 heteroring, and the hydrocarbon ring and the heteroring adjacent to each other may bond to each other to form a fused ring.

In one embodiment of the present disclosure, Ar1 and Ar2 are the same as or different from each other, and may be each independently a substituted or unsubstituted C6 to C60 aryl group; or a substituted or unsubstituted C2 to C60 heteroaryl group.

In another embodiment of the present disclosure, Ar1 and Ar2 are the same as or different from each other, and may be each independently a substituted or unsubstituted C6 to C30 aryl group; or a substituted or unsubstituted C2 to C30 heteroaryl group.

In another embodiment of the present disclosure, Ar1 and Ar2 are the same as or different from each other, and may be each independently a substituted or unsubstituted C6 to C20 aryl group; or a substituted or unsubstituted C2 to C20 heteroaryl group.

In another embodiment of the present disclosure, Ar1 and Ar2 are the same as or different from each other, and may be each independently a substituted or unsubstituted phenyl group; a substituted or unsubstituted biphenyl group; a substituted or unsubstituted terphenyl group; a substituted or unsubstituted naphthyl group; a substituted or unsubstituted fluorenyl group; a substituted or unsubstituted spirobifluorenyl group; a substituted or unsubstituted phenanthrenyl group; a substituted or unsubstituted pyrenyl group; a substituted or unsubstituted dibenzofuranyl group; or a substituted or unsubstituted dibenzothiophenyl group.

In one embodiment of the present disclosure, Ra is hydrogen; deuterium; halogen; a cyano group; a substituted or unsubstituted C1 to C30 alkyl group; a substituted or unsubstituted C2 to C30 alkenyl group; a substituted or unsubstituted C2 to C30 alkynyl group; a substituted or unsubstituted C1 to C30 alkoxy group; a substituted or unsubstituted C3 to C30 cycloalkyl group; a substituted or unsubstituted C2 to C30 heterocycloalkyl group; a substituted or unsubstituted C6 to C30 aryl group; a substituted or unsubstituted C2 to C30 heteroaryl group; —P(═O)R101R102; or —SiR101R102R103, wherein R101, R102 and R103 are the same as or different from each other and may be each independently a substituted or unsubstituted C1 to C30 alkyl group; a substituted or unsubstituted C6 to C30 aryl group; or a substituted or unsubstituted C2 to C30 heteroaryl group.

In another embodiment of the present disclosure, Ra is hydrogen; deuterium; halogen; a cyano group; a substituted or unsubstituted C1 to C20 alkyl group; a substituted or unsubstituted C2 to C20 alkenyl group; a substituted or unsubstituted C2 to C20 alkynyl group; a substituted or unsubstituted C1 to C20 alkoxy group; a substituted or unsubstituted C3 to C20 cycloalkyl group; a substituted or unsubstituted C2 to C20 heterocycloalkyl group; a substituted or unsubstituted C6 to C20 aryl group; a substituted or unsubstituted C2 to C20 heteroaryl group; —P(═O)R101R102; or —SiR101R102R103, wherein R101, R102 and R103 are the same as or different from each other and may be each independently a substituted or unsubstituted C1 to C20 alkyl group; a substituted or unsubstituted C6 to C20 aryl group; or a substituted or unsubstituted C2 to C20 heteroaryl group.

In another embodiment of the present disclosure, Ra may be a substituted or unsubstituted C6 to C20 aryl group; or a substituted or unsubstituted C2 to C20 heteroaryl group.

In another embodiment of the present disclosure, Ra may be a substituted or unsubstituted phenyl group.

In one embodiment of the present disclosure, the A ring and the B ring are the same as or different from each other and may be each independently a substituted or unsubstituted C6 to C30 aryl ring; a substituted or unsubstituted C4 to C30 heteroaryl ring; a substituted or unsubstituted C5 to C30 cycloalkyl ring; or a substituted or unsubstituted C5 to C30 cycloalkenyl ring, two or more groups of substituents of the A ring and the B ring adjacent to each other may bond to each other to form a substituted or unsubstituted C5 to C60 hydrocarbon ring or a substituted or unsubstituted C4 to C60 heteroring, and the hydrocarbon ring and the heteroring adjacent to each other may further bond to each other to form a fused ring.

In another embodiment of the present disclosure, the A ring and the B ring are the same as or different from each other and may be each independently a substituted or unsubstituted C6 to C20 aryl ring; a substituted or unsubstituted C4 to C20 heteroaryl ring; a substituted or unsubstituted C5 to C20 cycloalkyl ring; or a substituted or unsubstituted C5 to C20 cycloalkenyl ring, two or more groups of substituents of the A ring and the B ring adjacent to each other may bond to each other to form a substituted or unsubstituted C5 to C60 hydrocarbon ring or a substituted or unsubstituted C4 to C60 heteroring, and the hydrocarbon ring and the heteroring adjacent to each other may further bond to each other to form a fused ring.

In another embodiment of the present disclosure, the A ring and the B ring are the same as or different from each other and may be each independently a substituted or unsubstituted C6 to C10 aryl ring; a substituted or unsubstituted C4 to C10 heteroaryl ring; a substituted or unsubstituted C5 to C10 cycloalkyl ring; or a substituted or unsubstituted C5 to C10 cycloalkenyl ring, two or more groups of substituents of the A ring and the B ring adjacent to each other may bond to each other to form a substituted or unsubstituted C5 to C60 hydrocarbon ring or a substituted or unsubstituted C4 to C60 heteroring, and the hydrocarbon ring and the heteroring adjacent to each other may further bond to each other to form a fused ring.

In one embodiment of the present disclosure, Chemical Formula 3-2 may be a group represented by any one of the following Chemical Formulae 3-2-1 to 3-2-7.

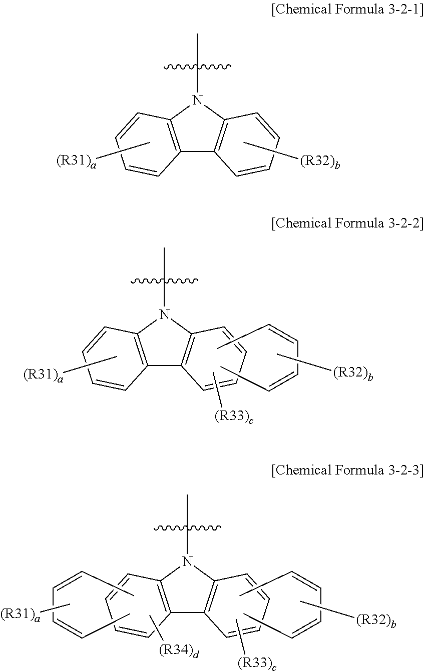

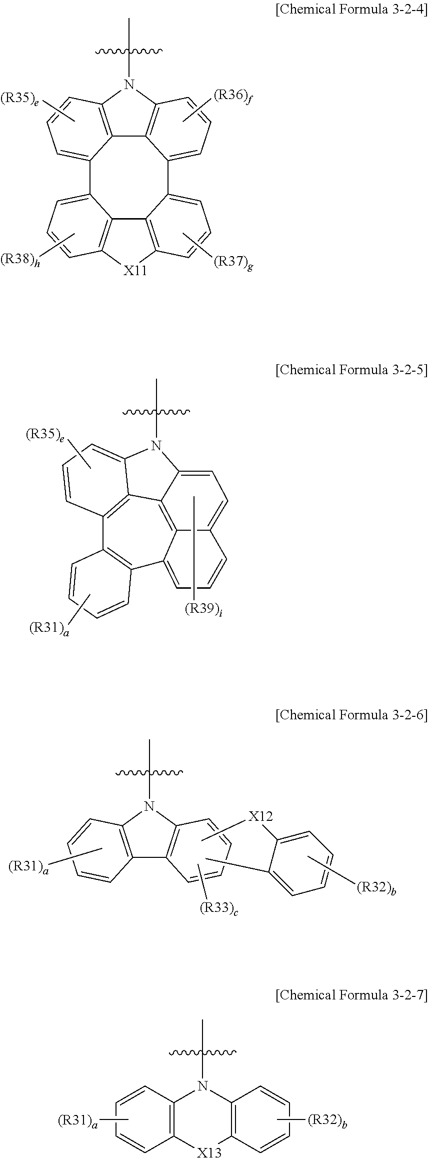

In Chemical Formulae 3-2-1 to 3-2-7,
X11 to X13 are the same as or different from each other, and each independently 0; S; NRb or CRcRd,
R31 to R39 and Rb to Rd are the same as or different from each other, and each independently selected from the group consisting of hydrogen; deuterium; halogen; a cyano group; a substituted or unsubstituted C1 to C60 alkyl group; a substituted or unsubstituted C2 to C60 alkenyl group; a substituted or unsubstituted C2 to C60 alkynyl group; a substituted or unsubstituted C1 to C60 alkoxy group; a substituted or unsubstituted C3 to C60 cycloalkyl group; a substituted or unsubstituted C2 to C60 heterocycloalkyl group; a substituted or unsubstituted C6 to C60 aryl group; a substituted or unsubstituted C2 to C60 heteroaryl group; —P(═O)R101R102; and —SiR101R102R103, or two or more groups adjacent to each other bond to each other to form a substituted or unsubstituted C6 to C60 aromatic hydrocarbon ring; or a substituted or unsubstituted C2 to C60 heteroring, wherein R101, R102 and R103 are the same as or different from each other and each independently a substituted or unsubstituted C1 to C60 alkyl group; a substituted or unsubstituted C6 to C60 aryl group; or a substituted or unsubstituted C2 to C60 heteroaryl group,
a and b are the same as or different from each other and each independently an integer of 0 to 4, and when a is 2 or greater, R31s are the same as or different from each other, and when b is 2 or greater, R32s are the same as or different from each other,
c and d are the same as or different from each other and each independently an integer of 0 to 2, and when c is 2 or greater, R33s are the same as or different from each other, and when d is 2 or greater, R34s are the same as or different from each other,
e to h are the same as or different from each other and each independently an integer of 0 to 3, and when e is 2 or greater, R35s are the same as or different from each other, when f is 2 or greater, R36s are the same as or different from each other, when g is 2 or greater, R37s are the same as or different from each other, and when h is 2 or greater, R38s are the same as or different from each other, and
i is an integer of 0 to 5, and when i is 2 or greater, R39s are the same as or different from each other.

In one embodiment of the present disclosure, R31 to R39 and Rb to Rd are the same as or different from each other, and each independently hydrogen; deuterium; halogen; a cyano group; a substituted or unsubstituted C1 to C30 alkyl group; a substituted or unsubstituted C2 to C30 alkenyl group; a substituted or unsubstituted C2 to C30 alkynyl group; a substituted or unsubstituted C1 to C30 alkoxy group; a substituted or unsubstituted C3 to C30 cycloalkyl group; a substituted or unsubstituted C2 to C30 heterocycloalkyl group; a substituted or unsubstituted C6 to C30 aryl group; a substituted or unsubstituted C2 to C30 heteroaryl group; —P(═O)R101R102; or —SiR101R102R103, or two or more groups adjacent to each other bond to each other to form a substituted or unsubstituted C6 to C30 aromatic hydrocarbon ring; or a substituted or unsubstituted C2 to C30 heteroring, wherein R101, R102 and R103 are the same as or different from each other and may be each independently a substituted or unsubstituted C1 to C30 alkyl group; a substituted or unsubstituted C6 to C30 aryl group; or a substituted or unsubstituted C2 to C30 heteroaryl group.

In another embodiment of the present disclosure, R31 to R39 and Rb to Rd are the same as or different from each other, and each independently hydrogen; deuterium; halogen; a cyano group; a substituted or unsubstituted C1 to C20 alkyl group; a substituted or unsubstituted C2 to C20 alkenyl group; a substituted or unsubstituted C2 to C20 alkynyl group; a substituted or unsubstituted C1 to C20 alkoxy group; a substituted or unsubstituted C3 to C20 cycloalkyl group; a substituted or unsubstituted C2 to C20 heterocycloalkyl group; a substituted or unsubstituted C6 to C20 aryl group; a substituted or unsubstituted C2 to C20 heteroaryl group; —P(═O)R101R102; or —SiR101R102R103, or two or more groups adjacent to each other bond to each other to form a substituted or unsubstituted C6 to C20 aromatic hydrocarbon ring; or a substituted or unsubstituted C2 to C20 heteroring, wherein R101, R102 and R103 are the same as or different from each other and may be each independently a substituted or unsubstituted C1 to C20 alkyl group; a substituted or unsubstituted C6 to C20 aryl group; or a substituted or unsubstituted C2 to C20 heteroaryl group.

In another embodiment of the present disclosure, R31 to R39 and Rb to Rd are the same as or different from each other, and each independently hydrogen; deuterium; a substituted or unsubstituted C1 to C20 alkyl group; a substituted or unsubstituted C6 to C20 aryl group; or a substituted or unsubstituted C2 to C20 heteroaryl group, or two or more groups adjacent to each other may bond to each other to form a substituted or unsubstituted C6 to C20 aromatic hydrocarbon ring; or a substituted or unsubstituted C2 to C20 heteroring.

In another embodiment of the present disclosure, R31 to R39 are the same as or different from each other, and each independently hydrogen; or deuterium, or two or more groups adjacent to each other may bond to each other to form a substituted or unsubstituted C6 to C20 aromatic hydrocarbon ring; or a substituted or unsubstituted C2 to C20 heteroring.

In another embodiment of the present disclosure, Rb may be a substituted or unsubstituted phenyl group.

In another embodiment of the present disclosure, Rc and Rd may each be a substituted or unsubstituted methyl group.

In one embodiment of the present disclosure, Chemical Formula 3-2-6 may be a group represented by any one of the following Chemical Formulae 3-2-6-1 and 3-2-6-2.

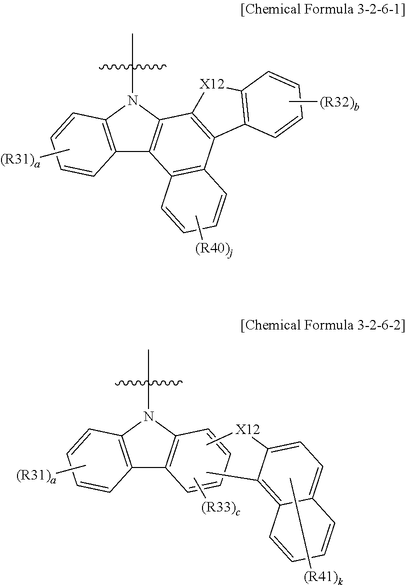

In Chemical Formulae 3-2-6-1 and 3-2-6-2,
R40 and R41 are the same as or different from each other and each independently selected from the group consisting of hydrogen; deuterium; halogen; a cyano group; a substituted or unsubstituted C1 to C60 alkyl group; a substituted or unsubstituted C2 to C60 alkenyl group; a substituted or unsubstituted C2 to C60 alkynyl group; a substituted or unsubstituted C1 to C60 alkoxy group; a substituted or unsubstituted C3 to C60 cycloalkyl group; a substituted or unsubstituted C2 to C60 heterocycloalkyl group; a substituted or unsubstituted C6 to C60 aryl group; a substituted or unsubstituted C2 to C60 heteroaryl group; —P(═O)R101R102; and —SiR101R102R103, wherein R101, R102 and R103 are the same as or different from each other and each independently a substituted or unsubstituted C1 to C60 alkyl group; a substituted or unsubstituted C6 to C60 aryl group; or a substituted or unsubstituted C2 to C60 heteroaryl group,
j is an integer of 0 to 4, and when j is 2 or greater, R40s are the same as or different from each other,
k is an integer of 0 to 6, and when k is 2 or greater, R41s are the same as or different from each other, and
X12, R31 to R33, and a to c have the same definitions as in Chemical Formula 3-2-6.

In one embodiment of the present disclosure, R40 and R41 are the same as or different from each other and each independently hydrogen; deuterium; halogen; a cyano group; a substituted or unsubstituted C1 to C30 alkyl group; a substituted or unsubstituted C2 to C30 alkenyl group; a substituted or unsubstituted C2 to C30 alkynyl group; a substituted or unsubstituted C1 to C30 alkoxy group; a substituted or unsubstituted C3 to C30 cycloalkyl group; a substituted or unsubstituted C2 to C30 heterocycloalkyl group; a substituted or unsubstituted C6 to C30 aryl group; a substituted or unsubstituted C2 to C30 heteroaryl group; —P(═O)R101R102; or —SiR101R102R103, wherein R101, R102 and R103 are the same as or different from each other and may be each independently a substituted or unsubstituted C1 to C30 alkyl group; a substituted or unsubstituted C6 to C30 aryl group; or a substituted or unsubstituted C2 to C30 heteroaryl group.

In another embodiment of the present disclosure, R40 and R41 are the same as or different from each other and each independently hydrogen; deuterium; halogen; a cyano group; a substituted or unsubstituted C1 to C20 alkyl group; a substituted or unsubstituted C2 to C20 alkenyl group; a substituted or unsubstituted C2 to C20 alkynyl group; a substituted or unsubstituted C1 to C20 alkoxy group; a substituted or unsubstituted C3 to C20 cycloalkyl group; a substituted or unsubstituted C2 to C20 heterocycloalkyl group; a substituted or unsubstituted C6 to C20 aryl group; a substituted or unsubstituted C2 to C20 heteroaryl group; —P(═O)R101R102; or —SiR101R102R103, wherein R101, R102 and R103 are the same as or different from each other and may be each independently a substituted or unsubstituted C1 to C20 alkyl group; a substituted or unsubstituted C6 to C20 aryl group; or a substituted or unsubstituted C2 to C20 heteroaryl group.

In another embodiment of the present disclosure, R40 and R41 are the same as or different from each other, and may be each independently hydrogen; deuterium; a substituted or unsubstituted C1 to C20 alkyl group; a substituted or unsubstituted C6 to C20 aryl group; or a substituted or unsubstituted C2 to C20 heteroaryl group.

In another embodiment of the present disclosure, R40 and R41 are the same as or different from each other, and may be each independently hydrogen; or deuterium.

In one embodiment of the present disclosure, Chemical Formula 3-2-7 may be a group represented by the following Chemical Formula 3-2-7-1.

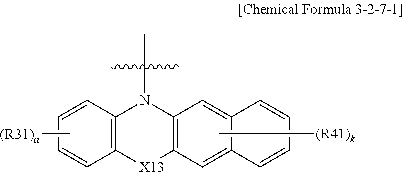

In Chemical Formula 3-2-7-1,
X13, R31 and a have the same definitions as in Chemical Formula 3-2-7, and
R41 and k have the same definitions as in Chemical Formula 3-2-6-2.

In one embodiment of the present disclosure, Chemical Formula 1 may be represented by any one of the following compounds.

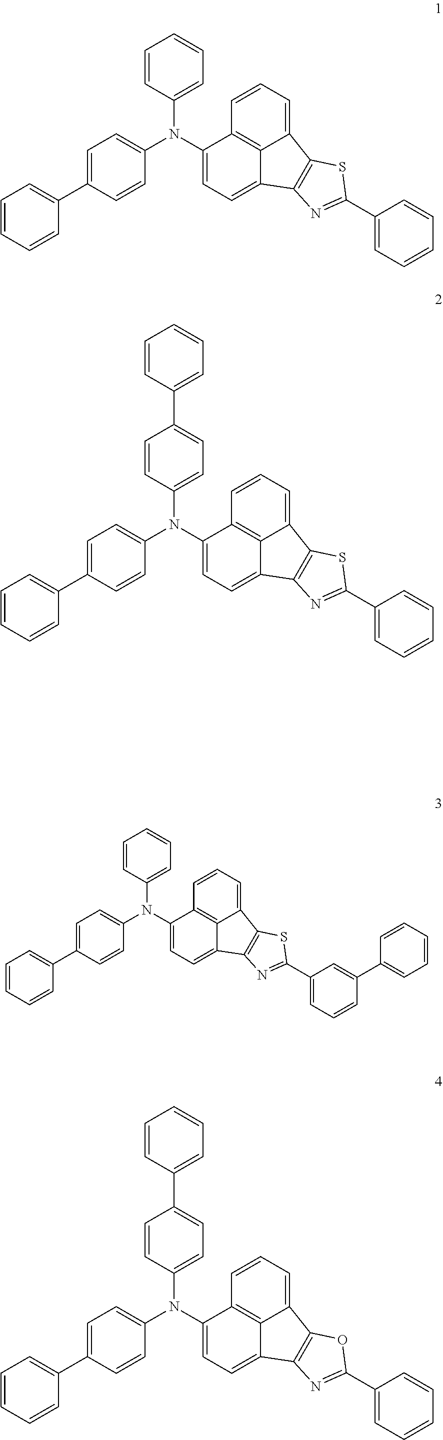 [SMILES: c1ccc(-c2ccc(N(c3ccccc3)c3ccc4c5c(cccc35)-c3sc(-c5ccccc5)nc3-4)cc2)cc1 ; c1ccc(-c2ccc(N(c3ccc(-c4ccccc4)cc3)c3ccc4c5c(cccc35)-c3sc(-c5ccccc5)nc3-4)cc2)cc1 ; c1ccc(-c2ccc(N(c3ccccc3)c3ccc4c5c(cccc35)-c3sc(-c5cccc(-c6ccccc6)c5)nc3-4)cc2)cc1 ; c1ccc(-c2ccc(N(c3ccc(-c4ccccc4)cc3)c3ccc4c5c(cccc35)-c3oc(-c5ccccc5)nc3-4)cc2)cc1]

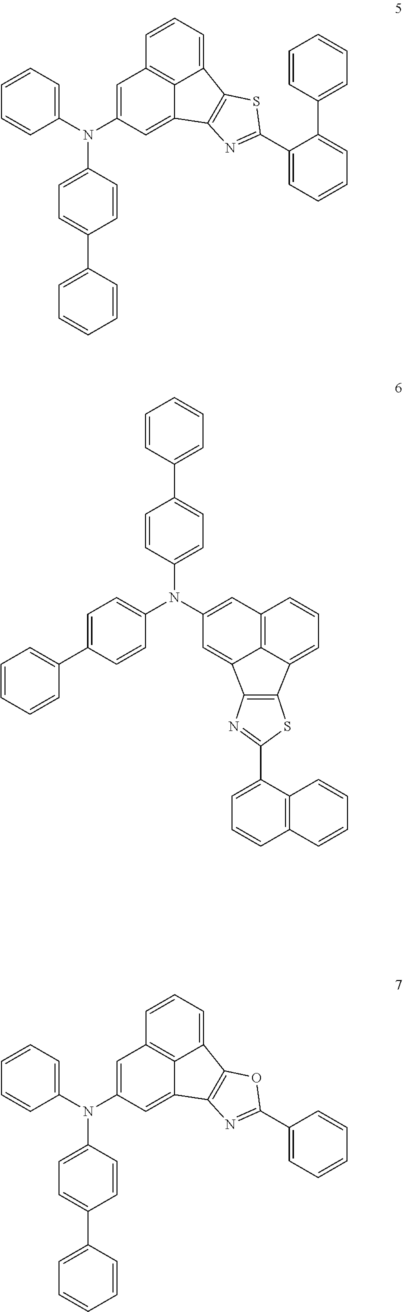 [SMILES: c1ccc(-c2ccc(N(c3ccccc3)c3cc4c5c(cccc5c3)-c3sc(-c5ccccc5-c5ccccc5)nc3-4)cc2)cc1 ; c1ccc(-c2ccc(N(c3ccc(-c4ccccc4)cc3)c3cc4c5c(cccc5c3)-c3sc(-c5cccc6ccccc56)nc3-4)cc2)cc1 ; c1ccc(-c2ccc(N(c3ccccc3)c3cc4c5c(cccc5c3)-c3oc(-c5ccccc5)nc3-4)cc2)cc1]

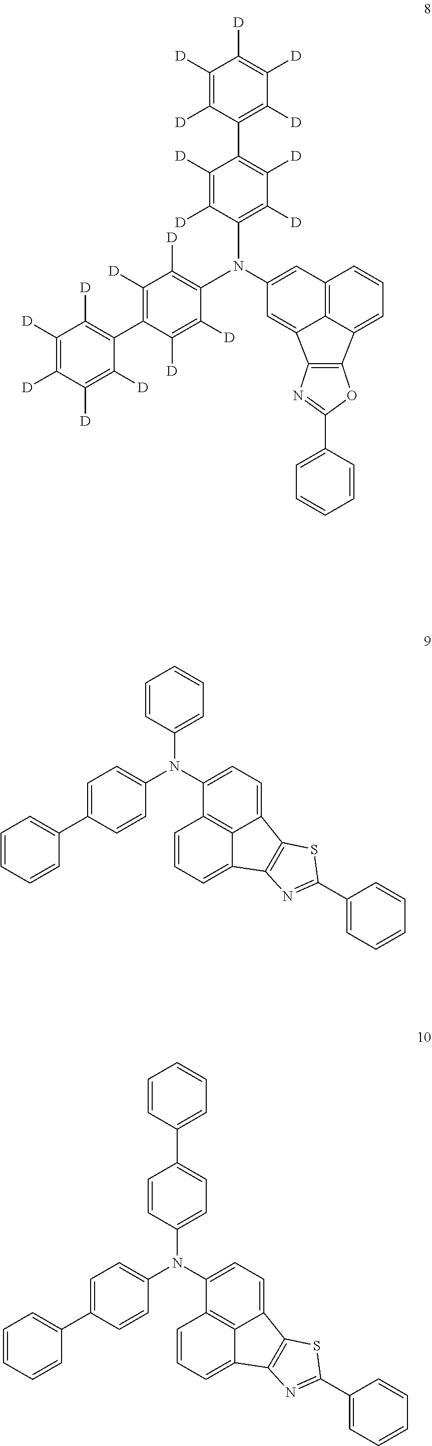 [SMILES: [2H]c1c([2H])c([2H])c(-c2c([2H])c([2H])c(N(c3cc4c5c(cccc5c3)-c3oc(-c5ccccc5)nc3-4)c3c([2H])c([2H])c(-c4c([2H])c([2H])c([2H])c([2H])c4[2H])c([2H])c3[2H])c([2H])c2[2H])c([2H])c1[2H] ; c1ccc(-c2ccc(N(c3ccccc3)c3ccc4c5c(cccc35)-c3nc(-c5ccccc5)sc3-4)cc2)cc1 ; c1ccc(-c2ccc(N(c3ccc(-c4ccccc4)cc3)c3ccc4c5c(cccc35)-c3nc(-c5ccccc5)sc3-4)cc2)cc1]

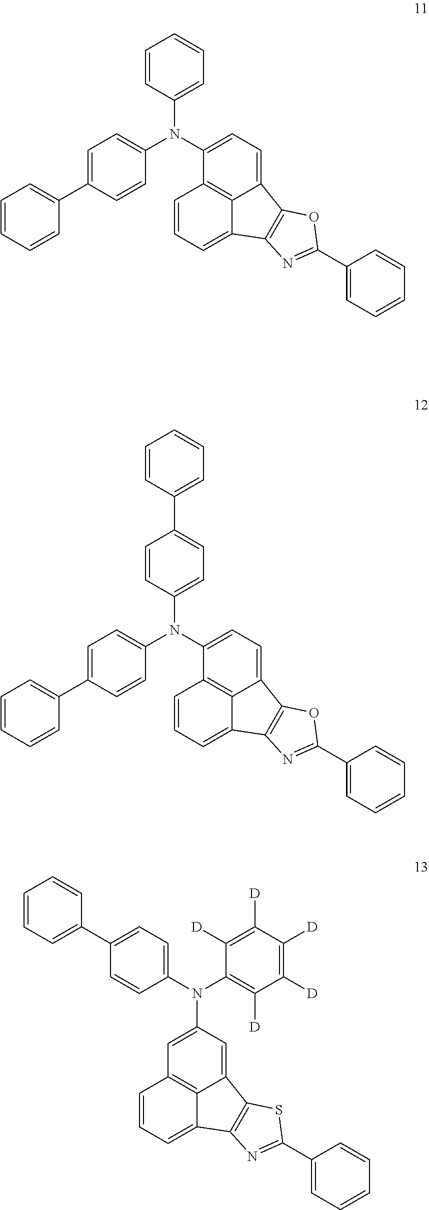 [SMILES: c1ccc(-c2ccc(N(c3ccccc3)c3ccc4c5c(cccc35)-c3nc(-c5ccccc5)oc3-4)cc2)cc1 ; c1ccc(-c2ccc(N(c3ccc(-c4ccccc4)cc3)c3ccc4c5c(cccc35)-c3nc(-c5ccccc5)oc3-4)cc2)cc1 ; [2H]c1c([2H])c([2H])c(N(c2ccc(-c3ccccc3)cc2)c2cc3c4c(cccc4c2)-c2nc(-c4ccccc4)sc2-3)c([2H])c1[2H]]

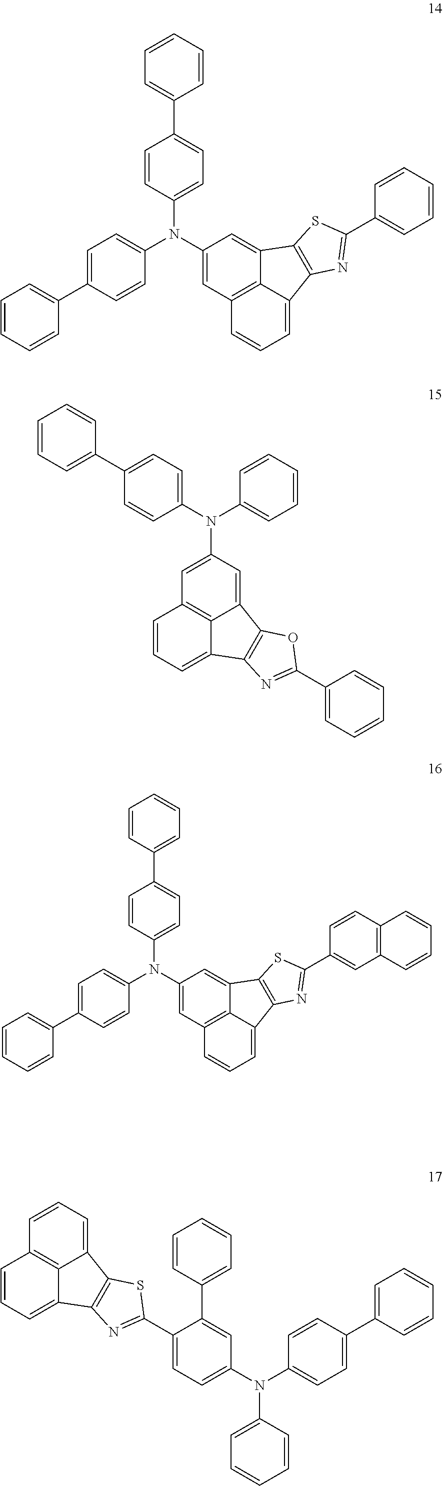 [SMILES: c1ccc(-c2ccc(N(c3ccc(-c4ccccc4)cc3)c3cc4c5c(cccc5c3)-c3nc(-c5ccccc5)sc3-4)cc2)cc1 ; c1ccc(-c2ccc(N(c3ccccc3)c3cc4c5c(cccc5c3)-c3nc(-c5ccccc5)oc3-4)cc2)cc1 ; c1ccc(-c2ccc(N(c3ccc(-c4ccccc4)cc3)c3cc4c5c(cccc5c3)-c3nc(-c5ccc6ccccc6c5)sc3-4)cc2)cc1 ; c1ccc(-c2ccc(N(c3ccccc3)c3ccc(-c4nc5c(s4)-c4cccc6cccc-5c46)c(-c4ccccc4)c3)cc2)cc1]

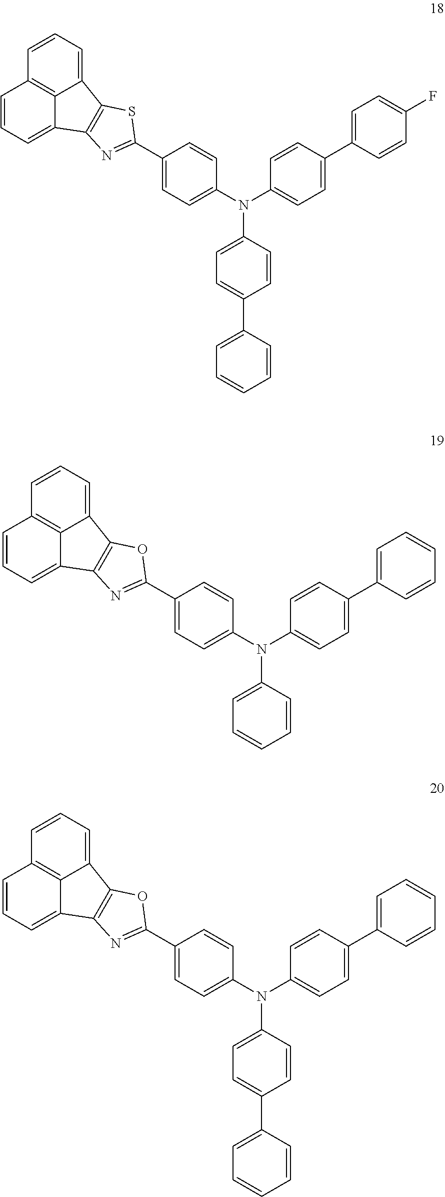 [SMILES: Fc1ccc(-c2ccc(N(c3ccc(-c4ccccc4)cc3)c3ccc(-c4nc5c(s4)-c4cccc6cccc-5c46)cc3)cc2)cc1 ; c1ccc(-c2ccc(N(c3ccccc3)c3ccc(-c4nc5c(o4)-c4cccc6cccc-5c46)cc3)cc2)cc1 ; c1ccc(-c2ccc(N(c3ccc(-c4ccccc4)cc3)c3ccc(-c4nc5c(o4)-c4cccc6cccc-5c46)cc3)cc2)cc1]

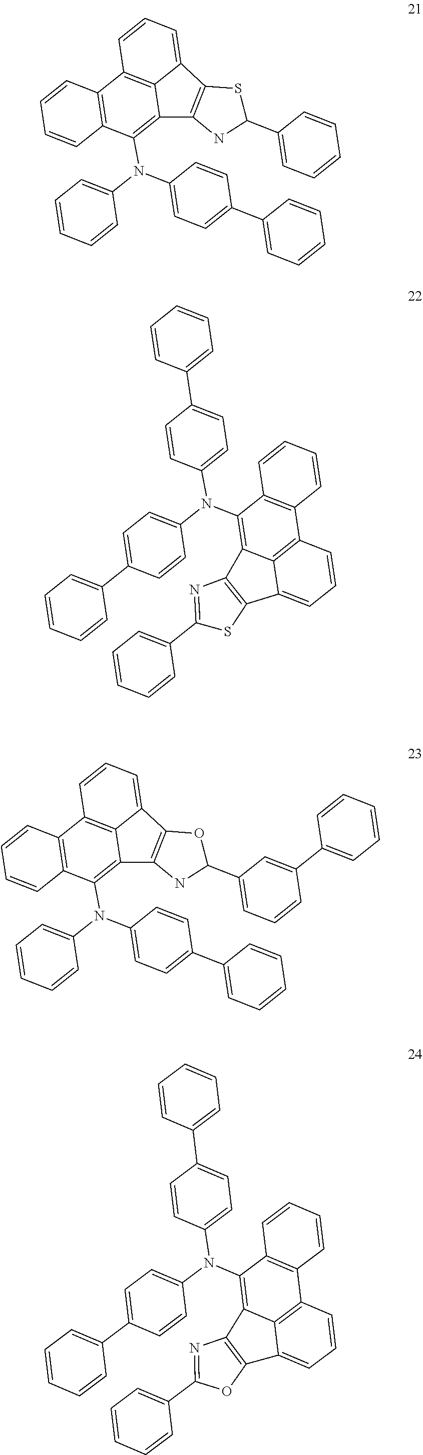

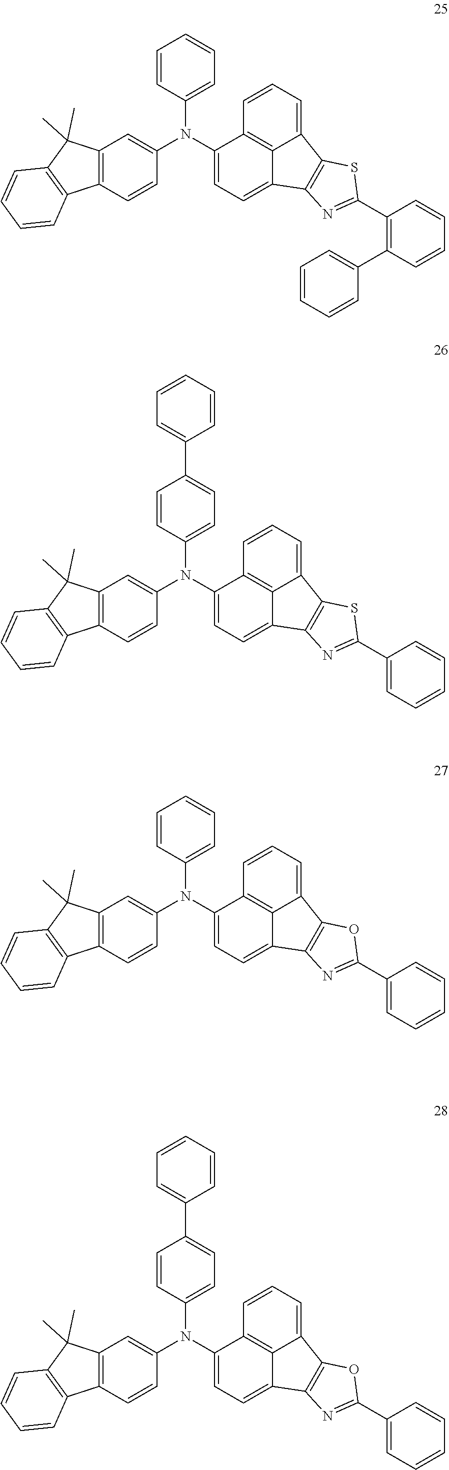 [SMILES: CC1(C)c2ccccc2-c2ccc(N(c3ccccc3)c3ccc4c5c(cccc35)-c3sc(-c5ccccc5-c5ccccc5)nc3-4)cc21 ; CC1(C)c2ccccc2-c2ccc(N(c3ccc(-c4ccccc4)cc3)c3ccc4c5c(cccc35)-c3sc(-c5ccccc5)nc3-4)cc21 ; CC1(C)c2ccccc2-c2ccc(N(c3ccccc3)c3ccc4c5c(cccc35)-c3oc(-c5ccccc5)nc3-4)cc21 ; CC1(C)c2ccccc2-c2ccc(N(c3ccc(-c4ccccc4)cc3)c3ccc4c5c(cccc35)-c3oc(-c5ccccc5)nc3-4)cc21]

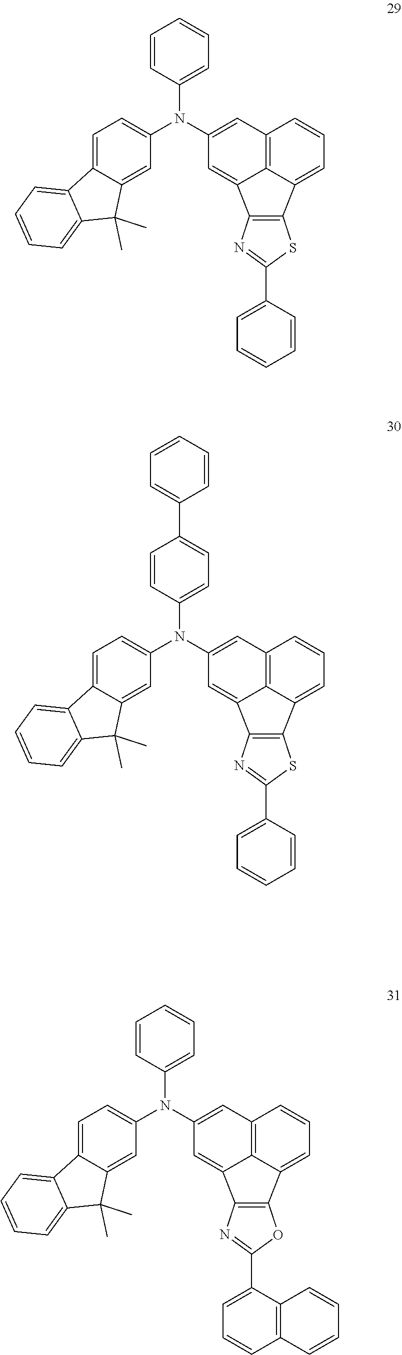 [SMILES: CC1(C)c2ccccc2-c2ccc(N(c3ccccc3)c3cc4c5c(cccc5c3)-c3sc(-c5ccccc5)nc3-4)cc21 ; CC1(C)c2ccccc2-c2ccc(N(c3ccc(-c4ccccc4)cc3)c3cc4c5c(cccc5c3)-c3sc(-c5ccccc5)nc3-4)cc21 ; CC1(C)c2ccccc2-c2ccc(N(c3ccccc3)c3cc4c5c(cccc5c3)-c3oc(-c5cccc6ccccc56)nc3-4)cc21]

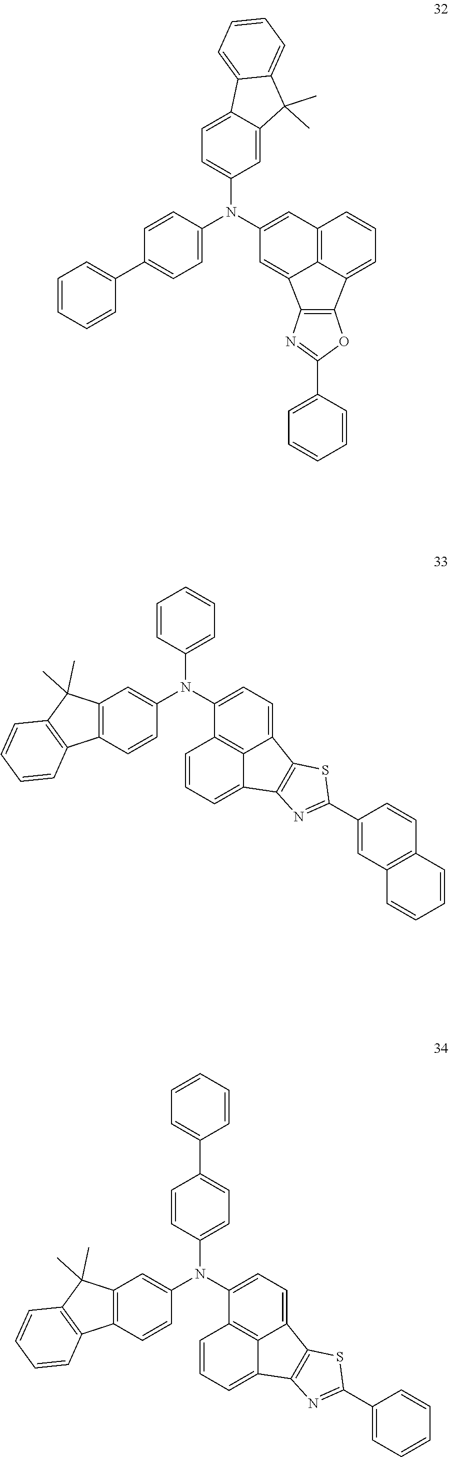 [SMILES: CC1(C)c2ccccc2-c2ccc(N(c3ccc(-c4ccccc4)cc3)c3cc4c5c(cccc5c3)-c3oc(-c5ccccc5)nc3-4)cc21 ; CC1(C)c2ccccc2-c2ccc(N(c3ccccc3)c3ccc4c5c(cccc35)-c3nc(-c5ccc6ccccc6c5)sc3-4)cc21 ; CC1(C)c2ccccc2-c2ccc(N(c3ccc(-c4ccccc4)cc3)c3ccc4c5c(cccc35)-c3nc(-c5ccccc5)sc3-4)cc21]

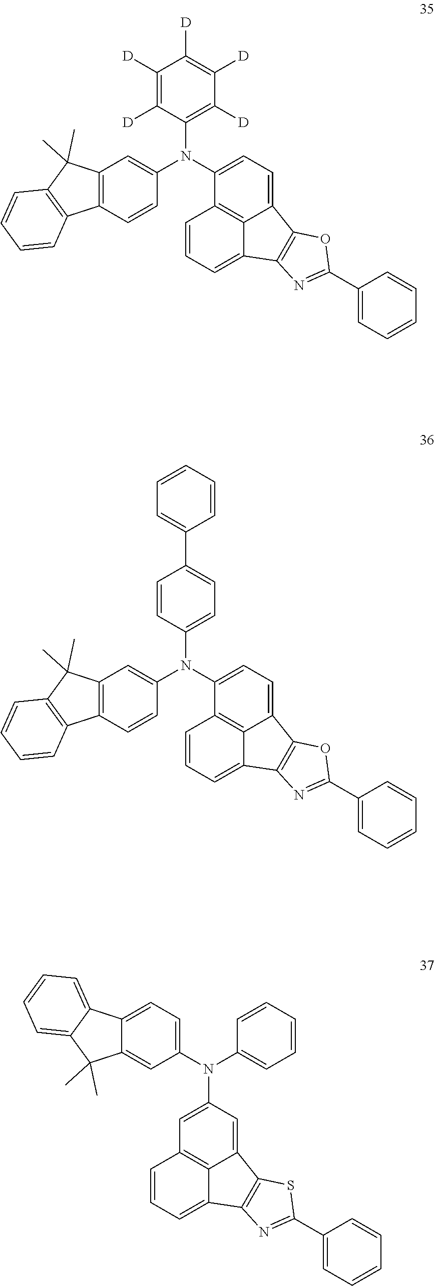 [SMILES: [2H]c1c([2H])c([2H])c(N(c2ccc3c(c2)C(C)(C)c2ccccc2-3)c2ccc3c4c(cccc24)-c2nc(-c4ccccc4)oc2-3)c([2H])c1[2H] ; CC1(C)c2ccccc2-c2ccc(N(c3ccc(-c4ccccc4)cc3)c3ccc4c5c(cccc35)-c3nc(-c5ccccc5)oc3-4)cc21 ; CC1(C)c2ccccc2-c2ccc(N(c3ccccc3)c3cc4c5c(cccc5c3)-c3nc(-c5ccccc5)sc3-4)cc21]

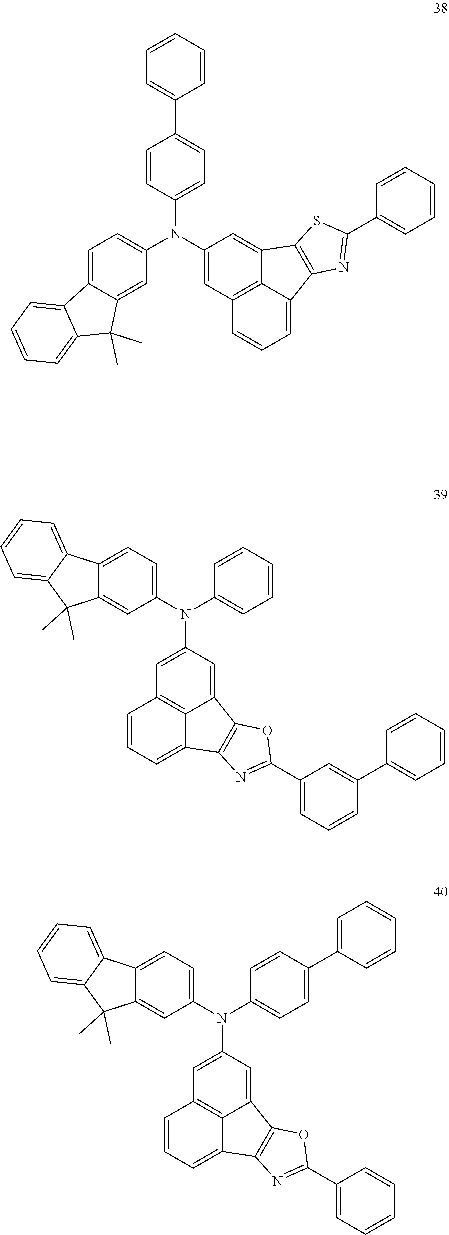 [SMILES: CC1(C)c2ccccc2-c2ccc(N(c3ccc(-c4ccccc4)cc3)c3cc4c5c(cccc5c3)-c3nc(-c5ccccc5)sc3-4)cc21 ; CC1(C)c2ccccc2-c2ccc(N(c3ccccc3)c3cc4c5c(cccc5c3)-c3nc(-c5cccc(-c6ccccc6)c5)oc3-4)cc21 ; CC1(C)c2ccccc2-c2ccc(N(c3ccc(-c4ccccc4)cc3)c3cc4c5c(cccc5c3)-c3nc(-c5ccccc5)oc3-4)cc21]

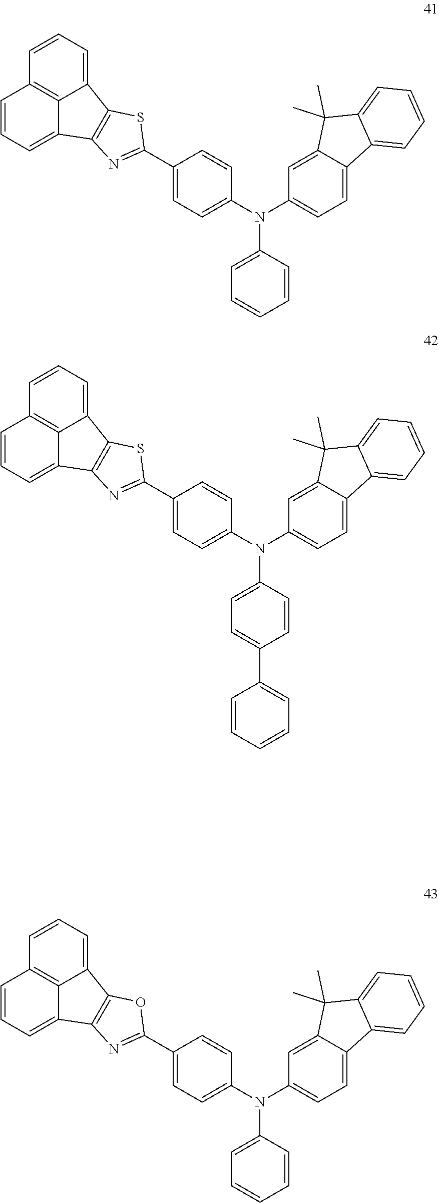 [SMILES: CC1(C)c2ccccc2-c2ccc(N(c3ccccc3)c3ccc(-c4nc5c(s4)-c4cccc6cccc-5c46)cc3)cc21 ; CC1(C)c2ccccc2-c2ccc(N(c3ccc(-c4ccccc4)cc3)c3ccc(-c4nc5c(s4)-c4cccc6cccc-5c46)cc3)cc21 ; CC1(C)c2ccccc2-c2ccc(N(c3ccccc3)c3ccc(-c4nc5c(o4)-c4cccc6cccc-5c46)cc3)cc21]

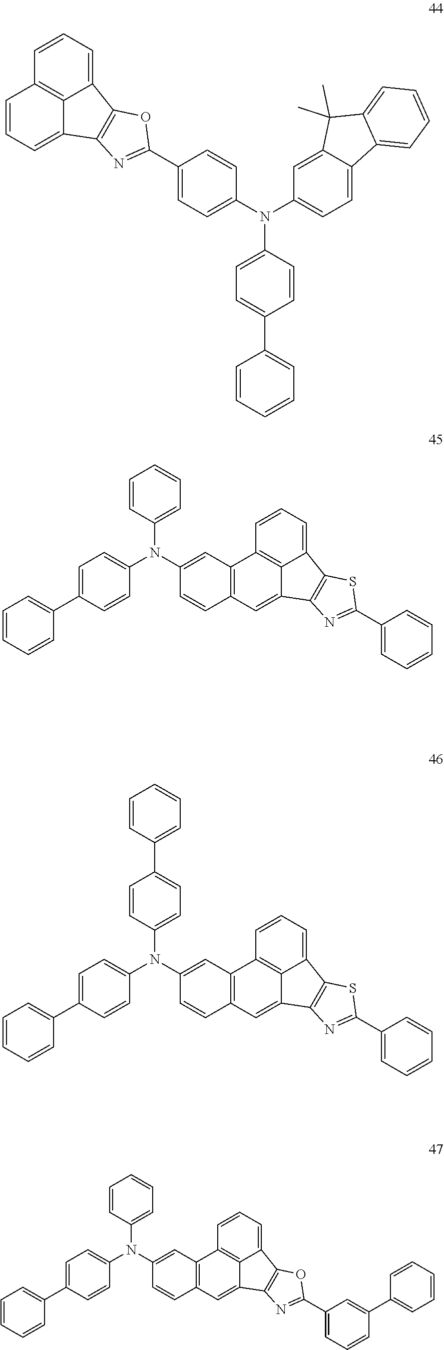 [SMILES: CC1(C)c2ccccc2-c2ccc(N(c3ccc(-c4ccccc4)cc3)c3ccc(-c4nc5c(o4)-c4cccc6cccc-5c46)cc3)cc21 ; c1ccc(-c2ccc(N(c3ccccc3)c3ccc4cc5c6c(cccc6c4c3)-c3sc(-c4ccccc4)nc3-5)cc2)cc1 ; c1ccc(-c2ccc(N(c3ccc(-c4ccccc4)cc3)c3ccc4cc5c6c(cccc6c4c3)-c3sc(-c4ccccc4)nc3-5)cc2)cc1 ; c1ccc(-c2ccc(N(c3ccccc3)c3ccc4cc5c6c(cccc6c4c3)-c3oc(-c4cccc(-c6ccccc6)c4)nc3-5)cc2)cc1]

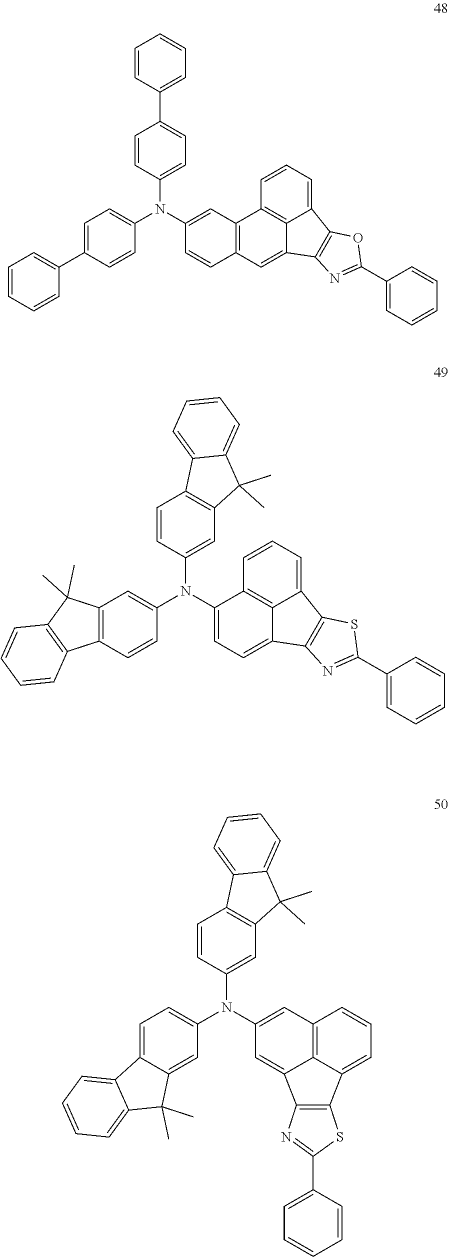 [SMILES: c1ccc(-c2ccc(N(c3ccc(-c4ccccc4)cc3)c3ccc4cc5c6c(cccc6c4c3)-c3oc(-c4ccccc4)nc3-5)cc2)cc1 ; CC1(C)c2ccccc2-c2ccc(N(c3ccc4c(c3)C(C)(C)c3ccccc3-4)c3ccc4c5c(cccc35)-c3sc(-c5ccccc5)nc3-4)cc21 ; CC1(C)c2ccccc2-c2ccc(N(c3ccc4c(c3)C(C)(C)c3ccccc3-4)c3cc4c5c(cccc5c3)-c3sc(-c5ccccc5)nc3-4)cc21]

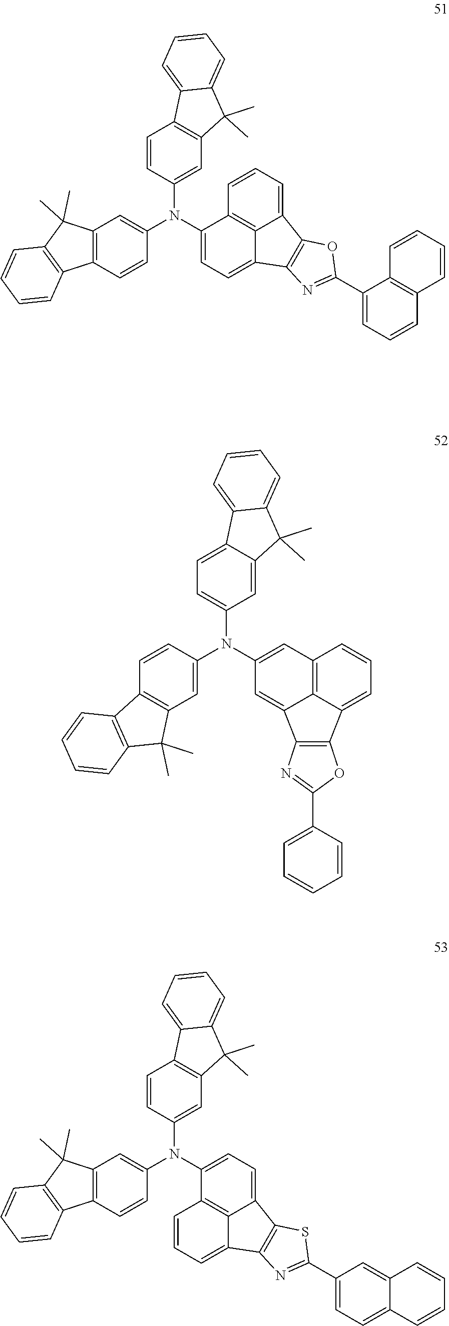 [SMILES: CC1(C)c2ccccc2-c2ccc(N(c3ccc4c(c3)C(C)(C)c3ccccc3-4)c3ccc4c5c(cccc35)-c3oc(-c5cccc6ccccc56)nc3-4)cc21 ; CC1(C)c2ccccc2-c2ccc(N(c3ccc4c(c3)C(C)(C)c3ccccc3-4)c3cc4c5c(cccc5c3)-c3oc(-c5ccccc5)nc3-4)cc21 ; CC1(C)c2ccccc2-c2ccc(N(c3ccc4c(c3)C(C)(C)c3ccccc3-4)c3ccc4c5c(cccc35)-c3nc(-c5ccc6ccccc6c5)sc3-4)cc21]

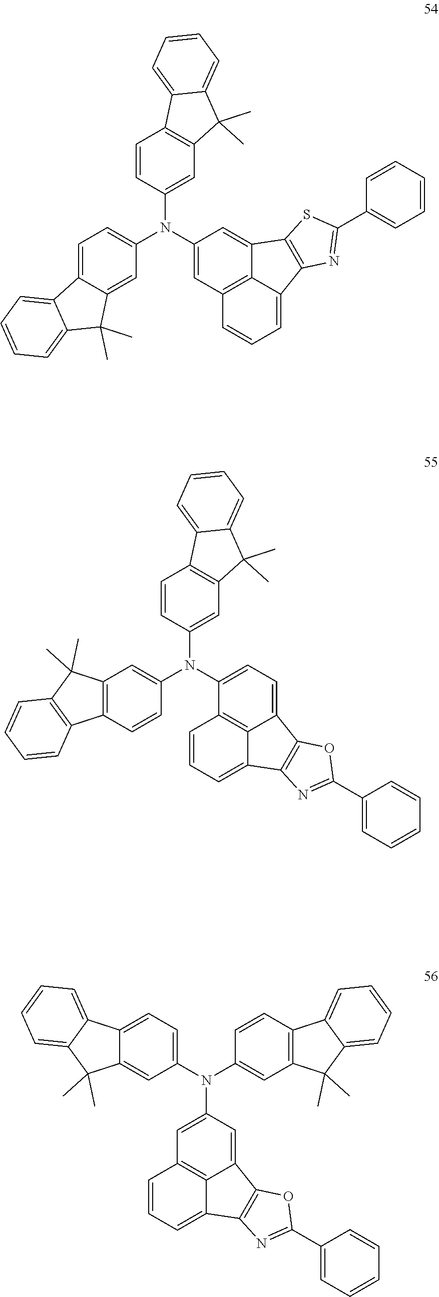 [SMILES: CC1(C)c2ccccc2-c2ccc(N(c3ccc4c(c3)C(C)(C)c3ccccc3-4)c3cc4c5c(cccc5c3)-c3nc(-c5ccccc5)sc3-4)cc21 ; CC1(C)c2ccccc2-c2ccc(N(c3ccc4c(c3)C(C)(C)c3ccccc3-4)c3ccc4c5c(cccc35)-c3nc(-c5ccccc5)oc3-4)cc21 ; CC1(C)c2ccccc2-c2ccc(N(c3ccc4c(c3)C(C)(C)c3ccccc3-4)c3cc4c5c(cccc5c3)-c3nc(-c5ccccc5)oc3-4)cc21]

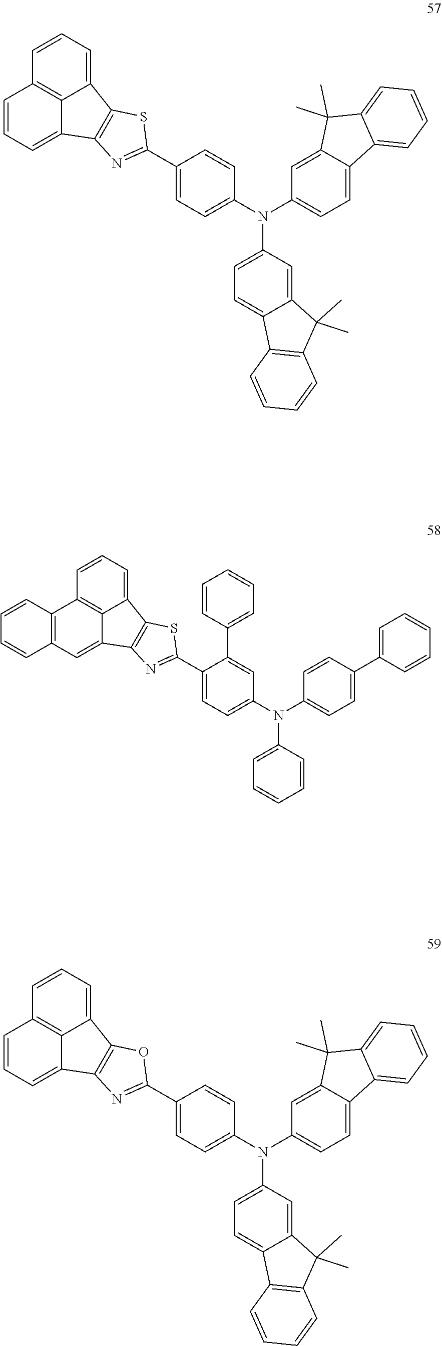 [SMILES: CC1(C)c2ccccc2-c2ccc(N(c3ccc(-c4nc5c(s4)-c4cccc6cccc-5c46)cc3)c3ccc4c(c3)C(C)(C)c3ccccc3-4)cc21 ; c1ccc(-c2ccc(N(c3ccccc3)c3ccc(-c4nc5c(s4)-c4cccc6c4c-5cc4ccccc46)c(-c4ccccc4)c3)cc2)cc1 ; CC1(C)c2ccccc2-c2ccc(N(c3ccc(-c4nc5c(o4)-c4cccc6cccc-5c46)cc3)c3ccc4c(c3)C(C)(C)c3ccccc3-4)cc21]

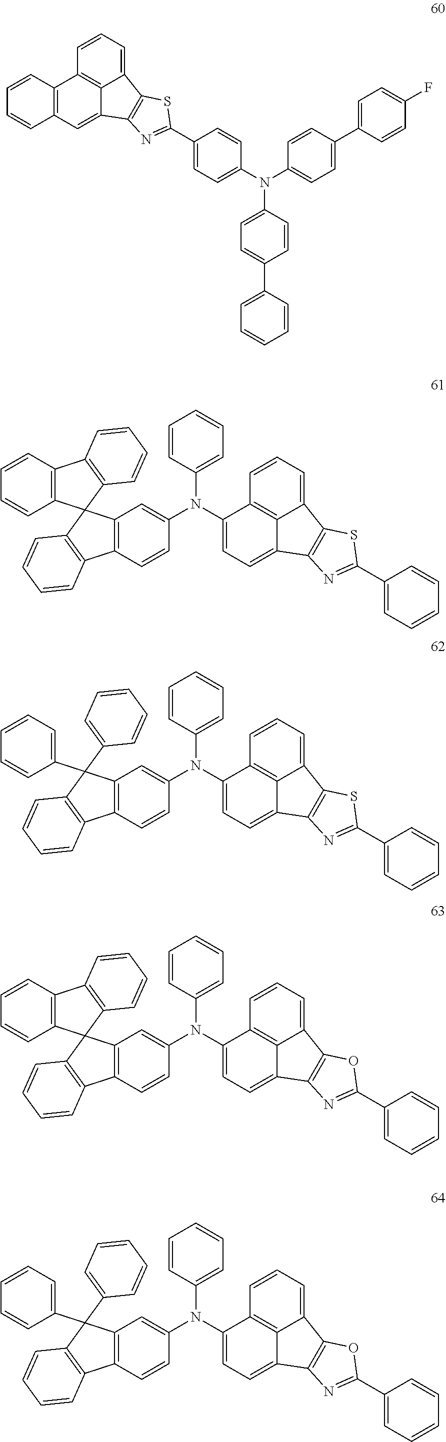 [SMILES: Fc1ccc(-c2ccc(N(c3ccc(-c4ccccc4)cc3)c3ccc(-c4nc5c(s4)-c4cccc6c4c-5cc4ccccc46)cc3)cc2)cc1 ; c1ccc(-c2nc3c(s2)-c2cccc4c(N(c5ccccc5)c5ccc6c(c5)C5(c7ccccc7-c7ccccc75)c5ccccc5-6)ccc-3c24)cc1 ; c1ccc(-c2nc3c(s2)-c2cccc4c(N(c5ccccc5)c5ccc6c(c5)C(c5ccccc5)(c5ccccc5)c5ccccc5-6)ccc-3c24)cc1 ; c1ccc(-c2nc3c(o2)-c2cccc4c(N(c5ccccc5)c5ccc6c(c5)C5(c7ccccc7-c7ccccc75)c5ccccc5-6)ccc-3c24)cc1 ; c1ccc(-c2nc3c(o2)-c2cccc4c(N(c5ccccc5)c5ccc6c(c5)C(c5ccccc5)(c5ccccc5)c5ccccc5-6)ccc-3c24)cc1]

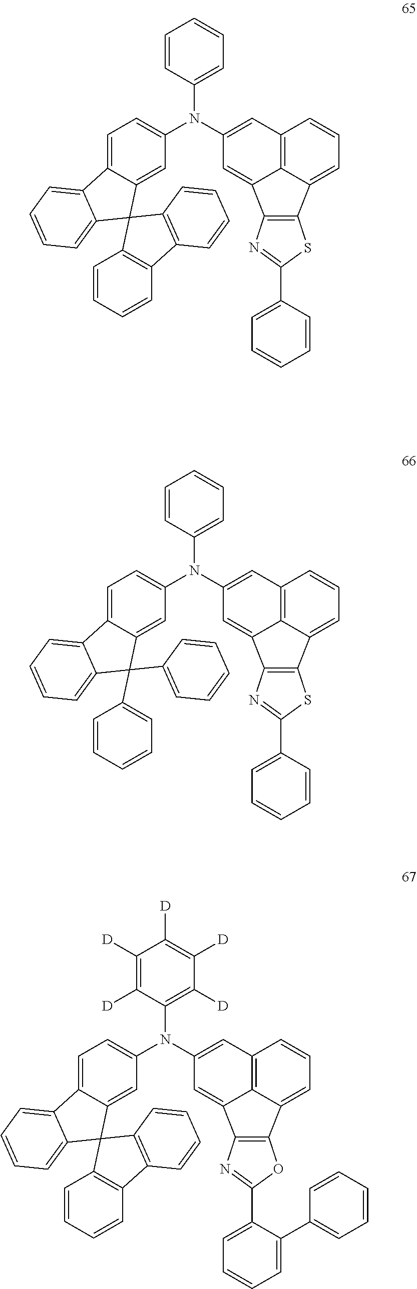 [SMILES: c1ccc(-c2nc3c(s2)-c2cccc4cc(N(c5ccccc5)c5ccc6c(c5)C5(c7ccccc7-c7ccccc75)c5ccccc5-6)cc-3c24)cc1 ; c1ccc(-c2nc3c(s2)-c2cccc4cc(N(c5ccccc5)c5ccc6c(c5)C(c5ccccc5)(c5ccccc5)c5ccccc5-6)cc-3c24)cc1 ; [2H]c1c([2H])c([2H])c(N(c2ccc3c(c2)C2(c4ccccc4-c4ccccc42)c2ccccc2-3)c2cc3c4c(cccc4c2)-c2oc(-c4ccccc4-c4ccccc4)nc2-3)c([2H])c1[2H]]

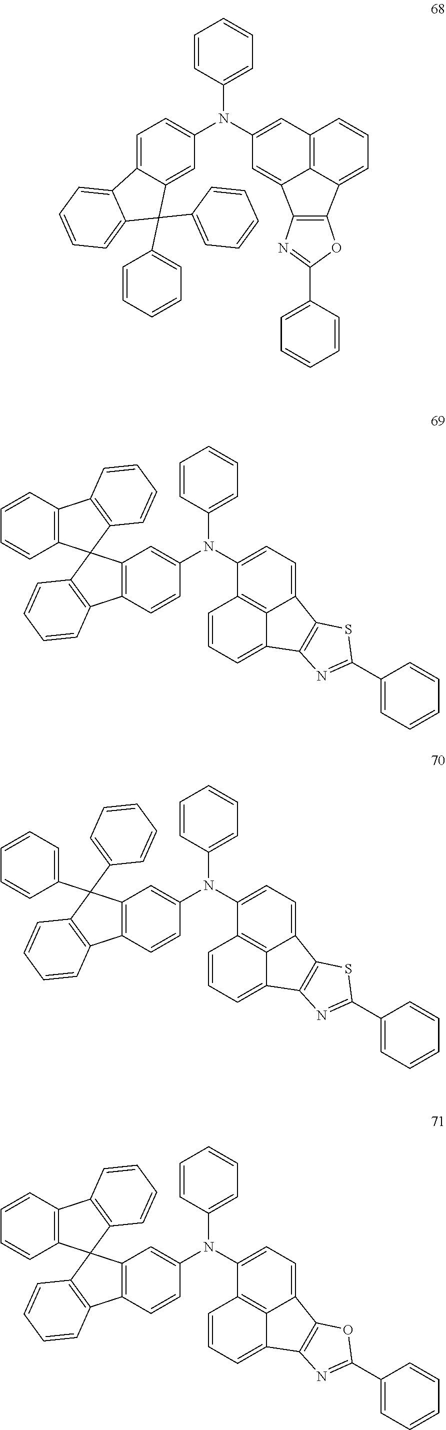 [SMILES: c1ccc(-c2nc3c(o2)-c2cccc4cc(N(c5ccccc5)c5ccc6c(c5)C(c5ccccc5)(c5ccccc5)c5ccccc5-6)cc-3c24)cc1 ; c1ccc(-c2nc3c(s2)-c2ccc(N(c4ccccc4)c4ccc5c(c4)C4(c6ccccc6-c6ccccc64)c4ccccc4-5)c4cccc-3c24)cc1 ; c1ccc(-c2nc3c(s2)-c2ccc(N(c4ccccc4)c4ccc5c(c4)C(c4ccccc4)(c4ccccc4)c4ccccc4-5)c4cccc-3c24)cc1 ; c1ccc(-c2nc3c(o2)-c2ccc(N(c4ccccc4)c4ccc5c(c4)C4(c6ccccc6-c6ccccc64)c4ccccc4-5)c4cccc-3c24)cc1]

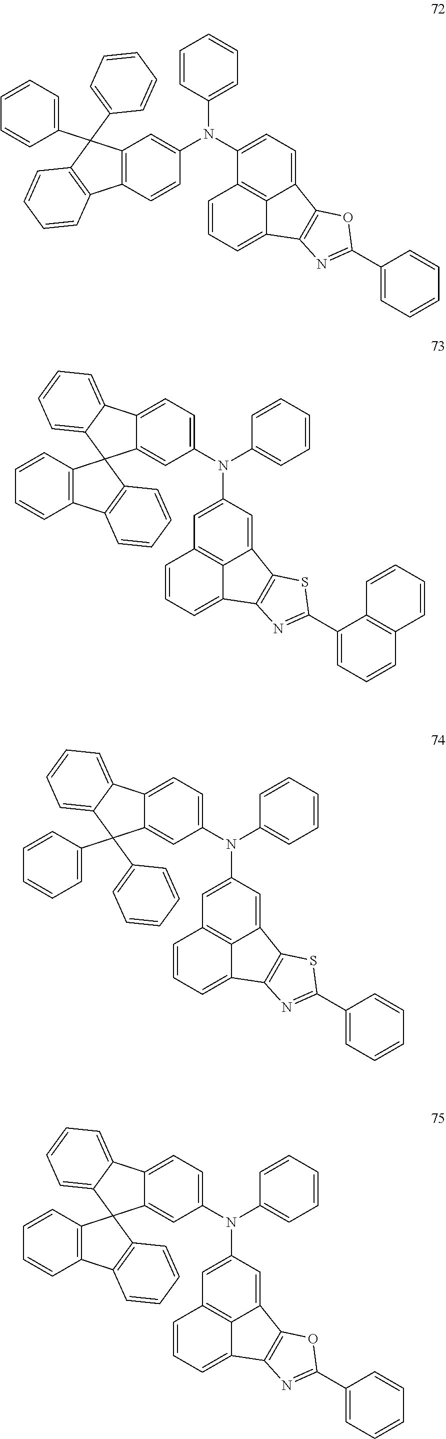 [SMILES: c1ccc(-c2nc3c(o2)-c2ccc(N(c4ccccc4)c4ccc5c(c4)C(c4ccccc4)(c4ccccc4)c4ccccc4-5)c4cccc-3c24)cc1 ; c1ccc(N(c2ccc3c(c2)C2(c4ccccc4-c4ccccc42)c2ccccc2-3)c2cc3c4c(cccc4c2)-c2nc(-c4cccc5ccccc45)sc2-3)cc1 ; c1ccc(-c2nc3c(s2)-c2cc(N(c4ccccc4)c4ccc5c(c4)C(c4ccccc4)(c4ccccc4)c4ccccc4-5)cc4cccc-3c24)cc1 ; c1ccc(-c2nc3c(o2)-c2cc(N(c4ccccc4)c4ccc5c(c4)C4(c6ccccc6-c6ccccc64)c4ccccc4-5)cc4cccc-3c24)cc1]

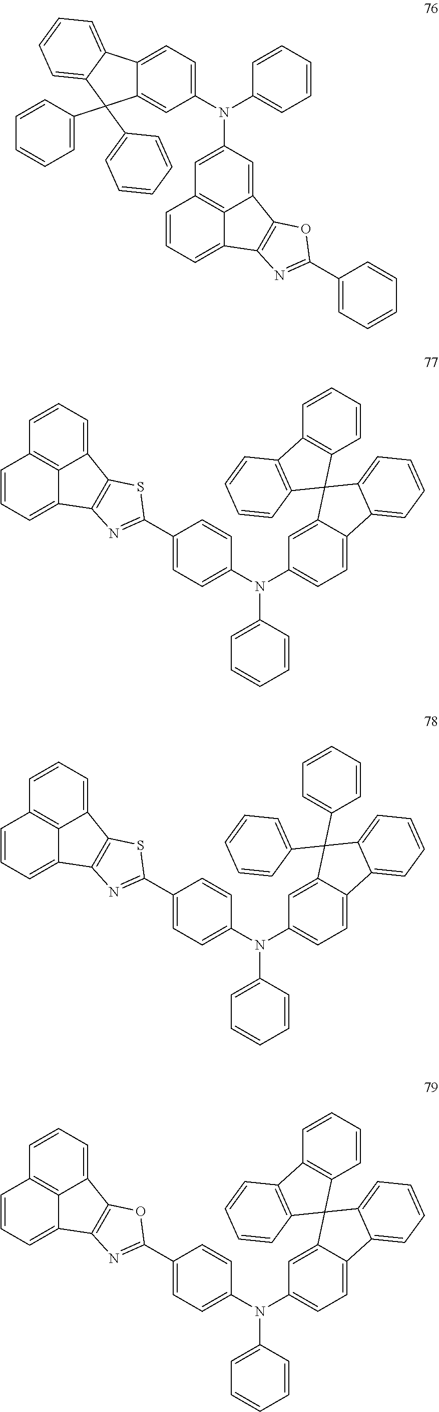 [SMILES: c1ccc(-c2nc3c(o2)-c2cc(N(c4ccccc4)c4ccc5c(c4)C(c4ccccc4)(c4ccccc4)c4ccccc4-5)cc4cccc-3c24)cc1 ; c1ccc(N(c2ccc(-c3nc4c(s3)-c3cccc5cccc-4c35)cc2)c2ccc3c(c2)C2(c4ccccc4-c4ccccc42)c2ccccc2-3)cc1 ; c1ccc(N(c2ccc(-c3nc4c(s3)-c3cccc5cccc-4c35)cc2)c2ccc3c(c2)C(c2ccccc2)(c2ccccc2)c2ccccc2-3)cc1 ; c1ccc(N(c2ccc(-c3nc4c(o3)-c3cccc5cccc-4c35)cc2)c2ccc3c(c2)C2(c4ccccc4-c4ccccc42)c2ccccc2-3)cc1]

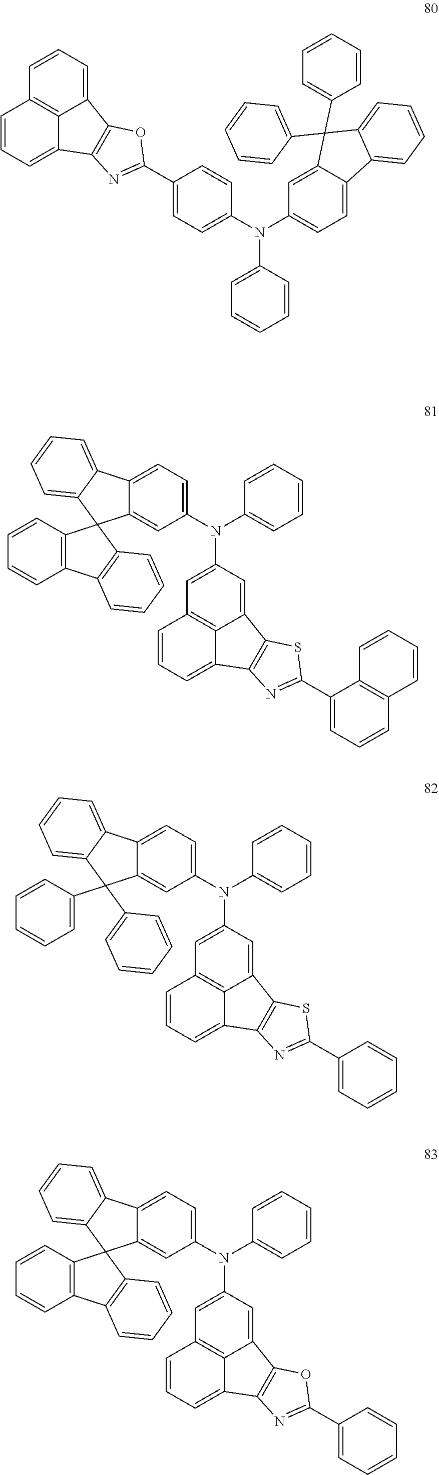 [SMILES: c1ccc(N(c2ccc(-c3nc4c(o3)-c3cccc5cccc-4c35)cc2)c2ccc3c(c2)C(c2ccccc2)(c2ccccc2)c2ccccc2-3)cc1 ; c1ccc(N(c2ccc3c(c2)C2(c4ccccc4-c4ccccc42)c2ccccc2-3)c2cc3c4c(cccc4c2)-c2nc(-c4cccc5ccccc45)sc2-3)cc1 ; c1ccc(-c2nc3c(s2)-c2cc(N(c4ccccc4)c4ccc5c(c4)C(c4ccccc4)(c4ccccc4)c4ccccc4-5)cc4cccc-3c24)cc1 ; c1ccc(-c2nc3c(o2)-c2cc(N(c4ccccc4)c4ccc5c(c4)C4(c6ccccc6-c6ccccc64)c4ccccc4-5)cc4cccc-3c24)cc1]

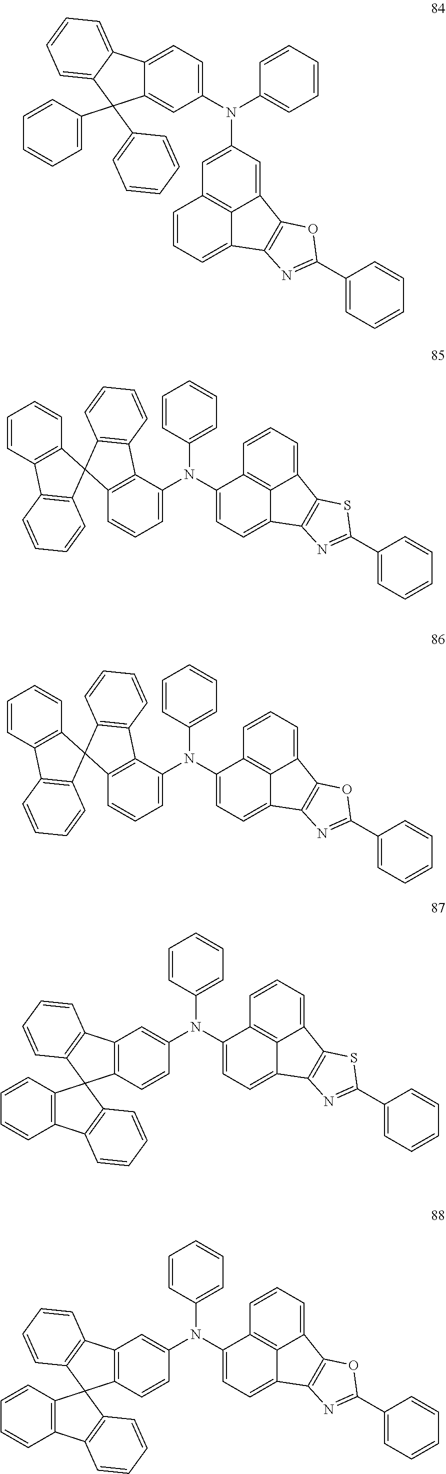 [SMILES: c1ccc(-c2nc3c(o2)-c2cc(N(c4ccccc4)c4ccc5c(c4)C(c4ccccc4)(c4ccccc4)c4ccccc4-5)cc4cccc-3c24)cc1 ; c1ccc(-c2nc3c(s2)-c2cccc4c(N(c5ccccc5)c5cccc6c5-c5ccccc5C65c6ccccc6-c6ccccc65)ccc-3c24)cc1 ; c1ccc(-c2nc3c(o2)-c2cccc4c(N(c5ccccc5)c5cccc6c5-c5ccccc5C65c6ccccc6-c6ccccc65)ccc-3c24)cc1 ; c1ccc(-c2nc3c(s2)-c2cccc4c(N(c5ccccc5)c5ccc6c(c5)-c5ccccc5C65c6ccccc6-c6ccccc65)ccc-3c24)cc1 ; c1ccc(-c2nc3c(o2)-c2cccc4c(N(c5ccccc5)c5ccc6c(c5)-c5ccccc5C65c6ccccc6-c6ccccc65)ccc-3c24)cc1]

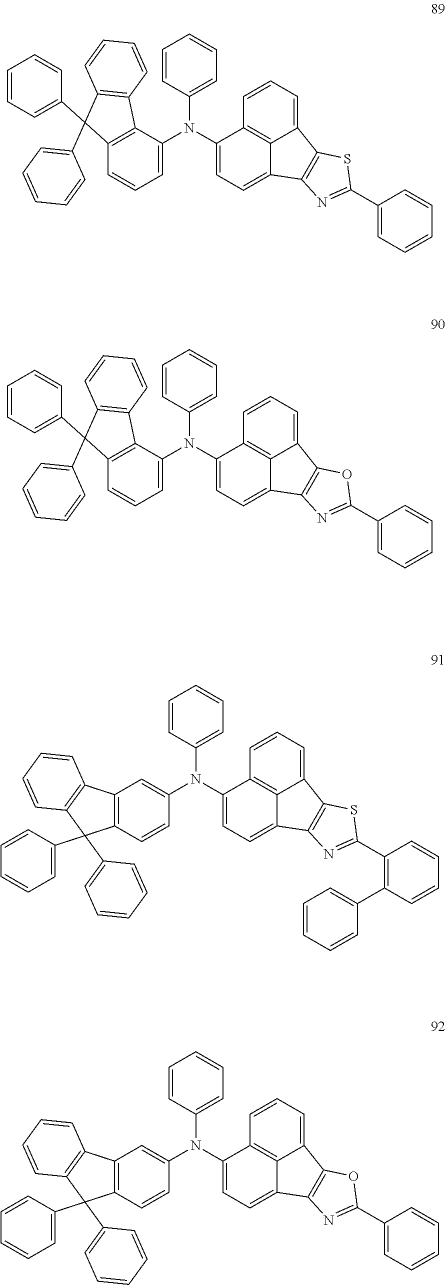 [SMILES: c1ccc(-c2nc3c(s2)-c2cccc4c(N(c5ccccc5)c5cccc6c5-c5ccccc5C6(c5ccccc5)c5ccccc5)ccc-3c24)cc1 ; c1ccc(-c2nc3c(o2)-c2cccc4c(N(c5ccccc5)c5cccc6c5-c5ccccc5C6(c5ccccc5)c5ccccc5)ccc-3c24)cc1 ; c1ccc(-c2ccccc2-c2nc3c(s2)-c2cccc4c(N(c5ccccc5)c5ccc6c(c5)-c5ccccc5C6(c5ccccc5)c5ccccc5)ccc-3c24)cc1 ; c1ccc(-c2nc3c(o2)-c2cccc4c(N(c5ccccc5)c5ccc6c(c5)-c5ccccc5C6(c5ccccc5)c5ccccc5)ccc-3c24)cc1]

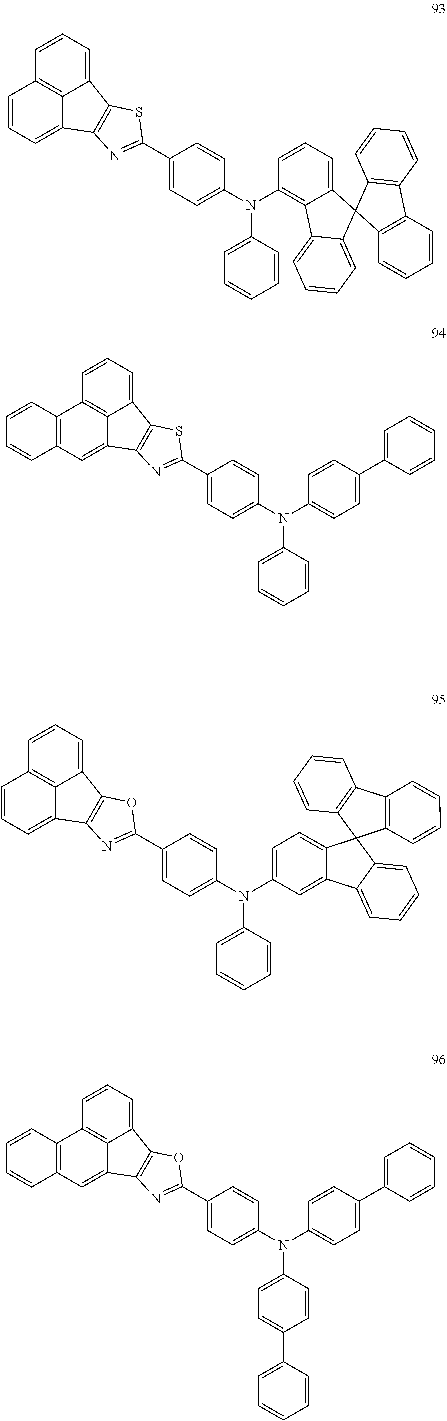 [SMILES: c1ccc(N(c2ccc(-c3nc4c(s3)-c3cccc5cccc-4c35)cc2)c2cccc3c2-c2ccccc2C32c3ccccc3-c3ccccc32)cc1 ; c1ccc(-c2ccc(N(c3ccccc3)c3ccc(-c4nc5c(s4)-c4cccc6c4c-5cc4ccccc46)cc3)cc2)cc1 ; c1ccc(N(c2ccc(-c3nc4c(o3)-c3cccc5cccc-4c35)cc2)c2ccc3c(c2)-c2ccccc2C32c3ccccc3-c3ccccc32)cc1 ; c1ccc(-c2ccc(N(c3ccc(-c4ccccc4)cc3)c3ccc(-c4nc5c(o4)-c4cccc6c4c-5cc4ccccc46)cc3)cc2)cc1]

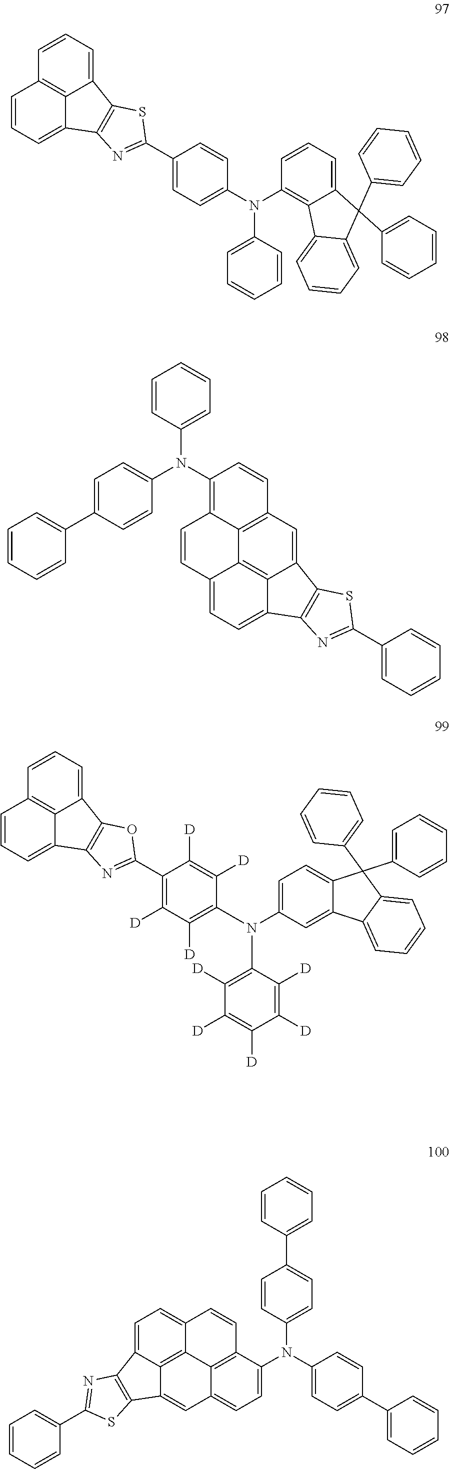 [SMILES: c1ccc(N(c2ccc(-c3nc4c(s3)-c3cccc5cccc-4c35)cc2)c2cccc3c2-c2ccccc2C3(c2ccccc2)c2ccccc2)cc1 ; c1ccc(-c2ccc(N(c3ccccc3)c3ccc4cc5c6c(ccc7ccc3c4c76)-c3nc(-c4ccccc4)sc3-5)cc2)cc1 ; [2H]c1c([2H])c([2H])c(N(c2ccc3c(c2)-c2ccccc2C3(c2ccccc2)c2ccccc2)c2c([2H])c([2H])c(-c3nc4c(o3)-c3cccc5cccc-4c35)c([2H])c2[2H])c([2H])c1[2H] ; c1ccc(-c2ccc(N(c3ccc(-c4ccccc4)cc3)c3ccc4cc5c6c(ccc7ccc3c4c76)-c3nc(-c4ccccc4)sc3-5)cc2)cc1]

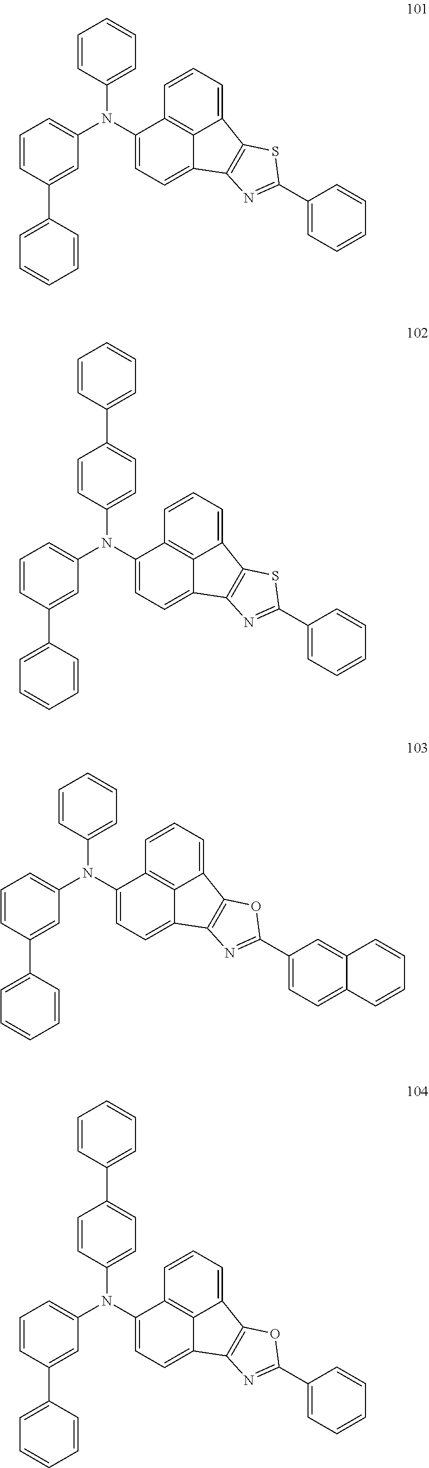 [SMILES: c1ccc(-c2cccc(N(c3ccccc3)c3ccc4c5c(cccc35)-c3sc(-c5ccccc5)nc3-4)c2)cc1 ; c1ccc(-c2ccc(N(c3cccc(-c4ccccc4)c3)c3ccc4c5c(cccc35)-c3sc(-c5ccccc5)nc3-4)cc2)cc1 ; c1ccc(-c2cccc(N(c3ccccc3)c3ccc4c5c(cccc35)-c3oc(-c5ccc6ccccc6c5)nc3-4)c2)cc1 ; c1ccc(-c2ccc(N(c3cccc(-c4ccccc4)c3)c3ccc4c5c(cccc35)-c3oc(-c5ccccc5)nc3-4)cc2)cc1]

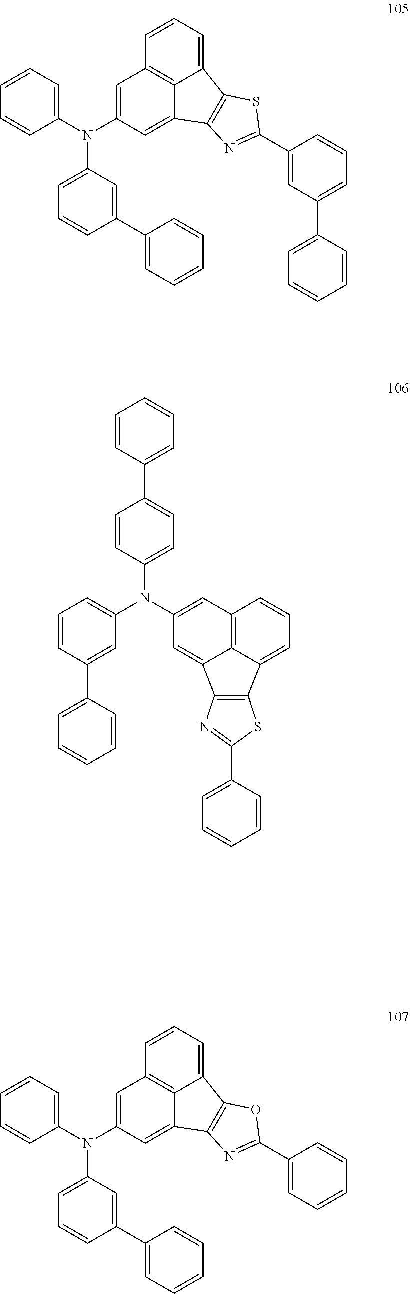 [SMILES: c1ccc(-c2cccc(-c3nc4c(s3)-c3cccc5cc(N(c6ccccc6)c6cccc(-c7ccccc7)c6)cc-4c35)c2)cc1 ; c1ccc(-c2ccc(N(c3cccc(-c4ccccc4)c3)c3cc4c5c(cccc5c3)-c3sc(-c5ccccc5)nc3-4)cc2)cc1 ; c1ccc(-c2cccc(N(c3ccccc3)c3cc4c5c(cccc5c3)-c3oc(-c5ccccc5)nc3-4)c2)cc1]

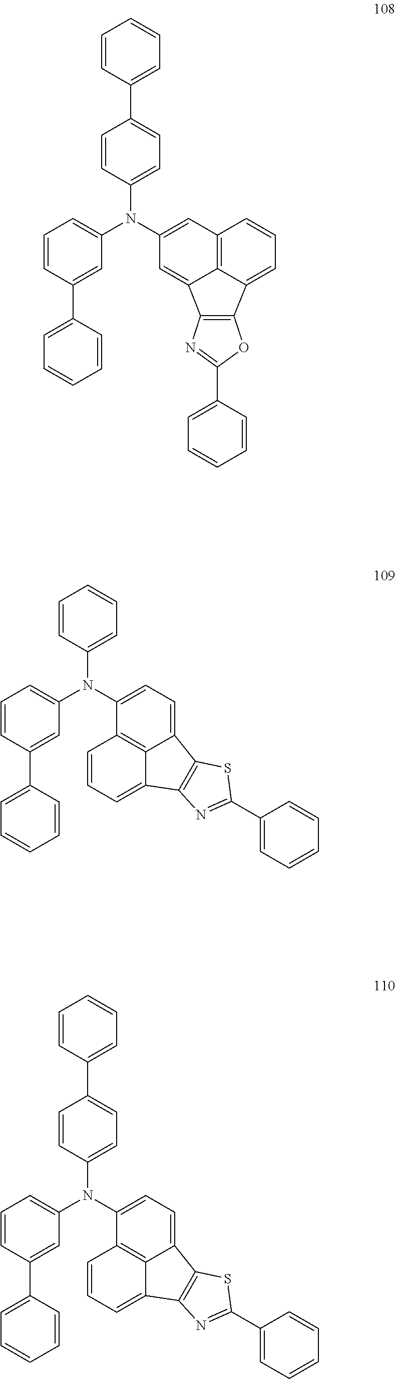 [SMILES: c1ccc(-c2ccc(N(c3cccc(-c4ccccc4)c3)c3cc4c5c(cccc5c3)-c3oc(-c5ccccc5)nc3-4)cc2)cc1 ; c1ccc(-c2cccc(N(c3ccccc3)c3ccc4c5c(cccc35)-c3nc(-c5ccccc5)sc3-4)c2)cc1 ; c1ccc(-c2ccc(N(c3cccc(-c4ccccc4)c3)c3ccc4c5c(cccc35)-c3nc(-c5ccccc5)sc3-4)cc2)cc1]

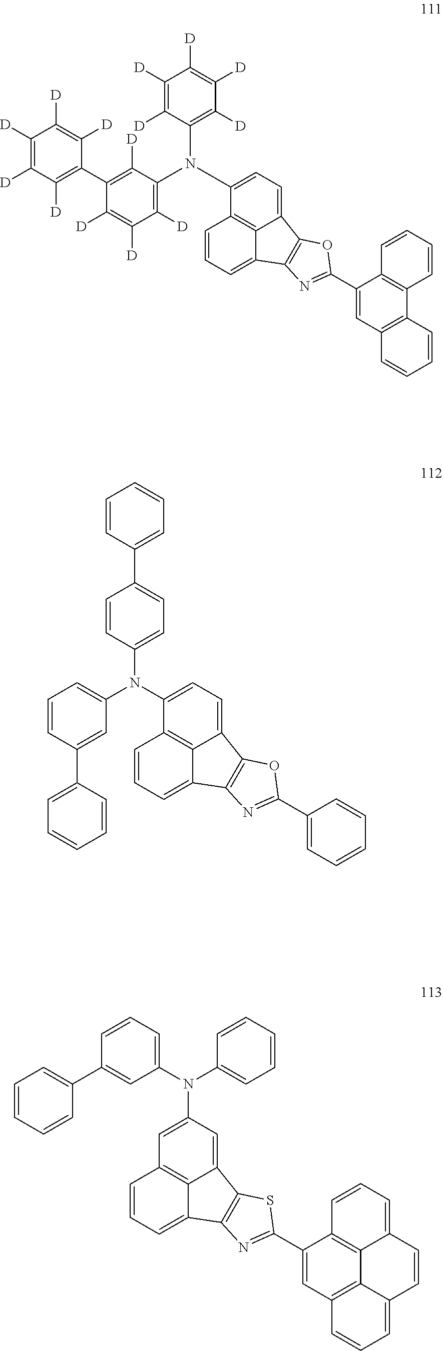 [SMILES: [2H]c1c([2H])c([2H])c(-c2c([2H])c([2H])c([2H])c(N(c3c([2H])c([2H])c([2H])c([2H])c3[2H])c3ccc4c5c(cccc35)-c3nc(-c5cc6ccccc6c6ccccc56)oc3-4)c2[2H])c([2H])c1[2H] ; c1ccc(-c2ccc(N(c3cccc(-c4ccccc4)c3)c3ccc4c5c(cccc35)-c3nc(-c5ccccc5)oc3-4)cc2)cc1 ; c1ccc(-c2cccc(N(c3ccccc3)c3cc4c5c(cccc5c3)-c3nc(-c5cc6cccc7ccc8cccc5c8c76)sc3-4)c2)cc1]

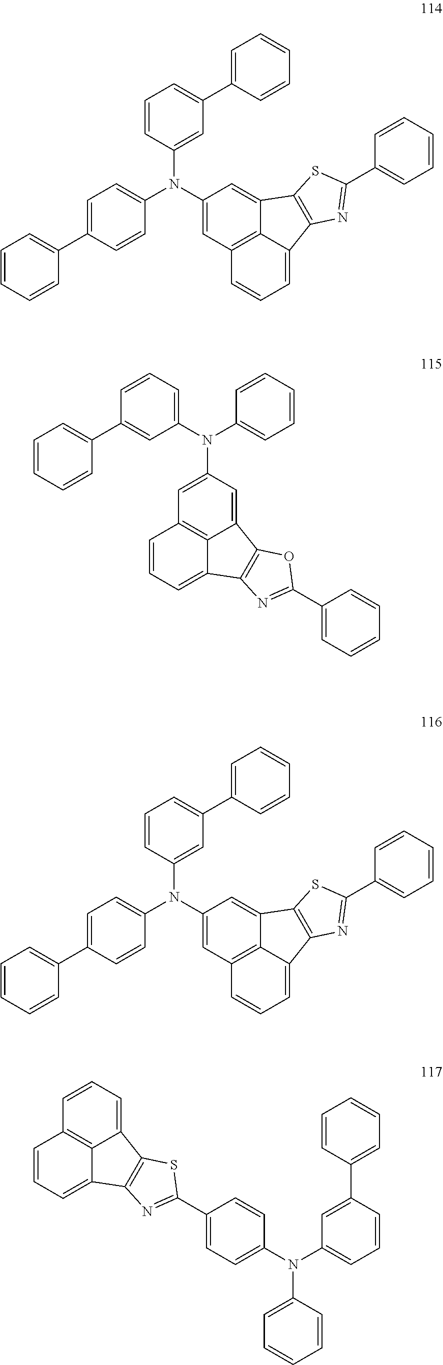 [SMILES: c1ccc(-c2ccc(N(c3cccc(-c4ccccc4)c3)c3cc4c5c(cccc5c3)-c3nc(-c5ccccc5)sc3-4)cc2)cc1 ; c1ccc(-c2cccc(N(c3ccccc3)c3cc4c5c(cccc5c3)-c3nc(-c5ccccc5)oc3-4)c2)cc1 ; c1ccc(-c2ccc(N(c3cccc(-c4ccccc4)c3)c3cc4c5c(cccc5c3)-c3nc(-c5ccccc5)sc3-4)cc2)cc1 ; c1ccc(-c2cccc(N(c3ccccc3)c3ccc(-c4nc5c(s4)-c4cccc6cccc-5c46)cc3)c2)cc1]

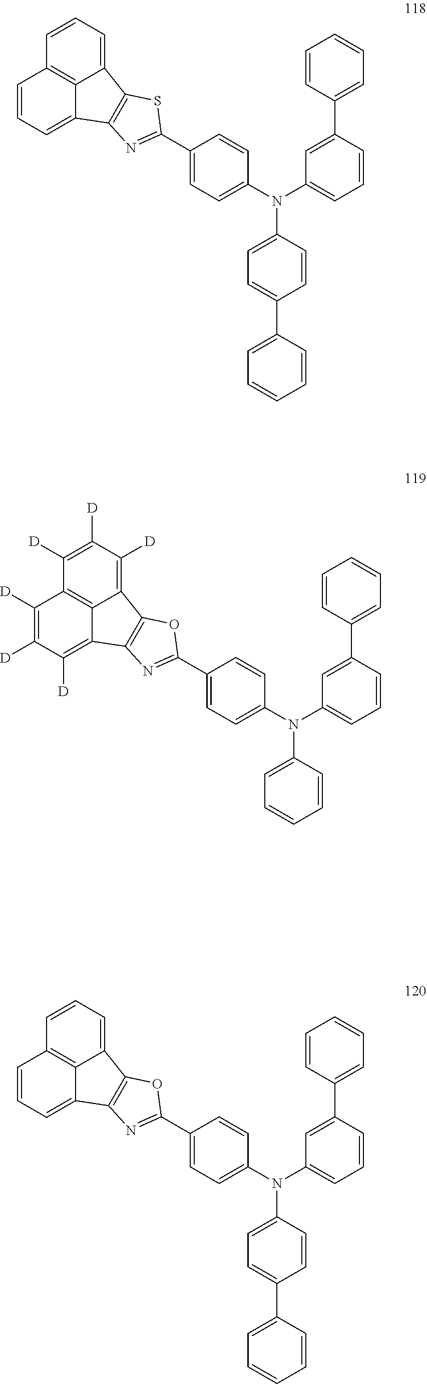 [SMILES: c1ccc(-c2ccc(N(c3ccc(-c4nc5c(s4)-c4cccc6cccc-5c46)cc3)c3cccc(-c4ccccc4)c3)cc2)cc1 ; [2H]c1c([2H])c2c3c(c([2H])c([2H])c([2H])c3c1[2H])-c1oc(-c3ccc(N(c4ccccc4)c4cccc(-c5ccccc5)c4)cc3)nc1-2 ; c1ccc(-c2ccc(N(c3ccc(-c4nc5c(o4)-c4cccc6cccc-5c46)cc3)c3cccc(-c4ccccc4)c3)cc2)cc1]

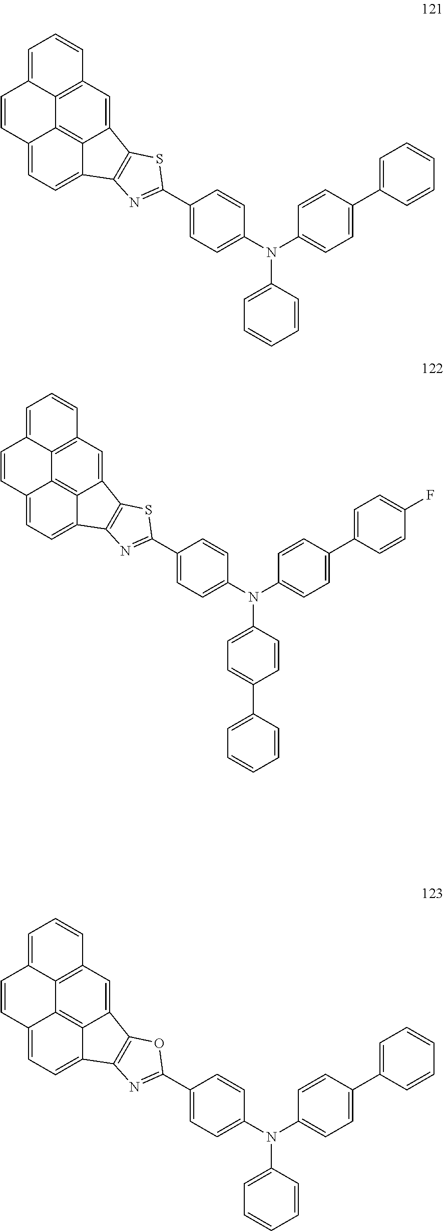 [SMILES: c1ccc(-c2ccc(N(c3ccccc3)c3ccc(-c4nc5c(s4)-c4cc6cccc7ccc8ccc-5c4c8c76)cc3)cc2)cc1 ; Fc1ccc(-c2ccc(N(c3ccc(-c4ccccc4)cc3)c3ccc(-c4nc5c(s4)-c4cc6cccc7ccc8ccc-5c4c8c76)cc3)cc2)cc1 ; c1ccc(-c2ccc(N(c3ccccc3)c3ccc(-c4nc5c(o4)-c4cc6cccc7ccc8ccc-5c4c8c76)cc3)cc2)cc1]

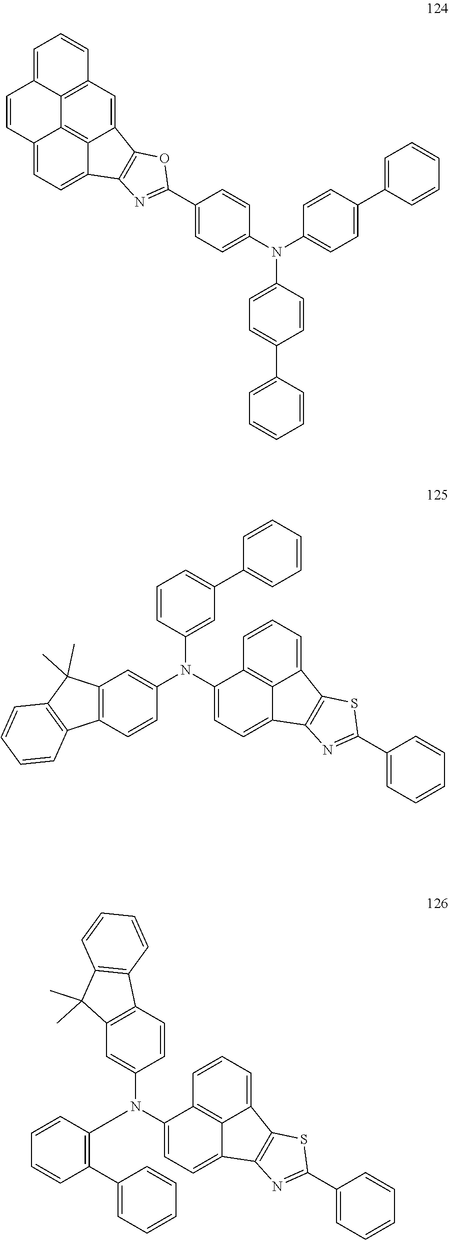 [SMILES: c1ccc(-c2ccc(N(c3ccc(-c4ccccc4)cc3)c3ccc(-c4nc5c(o4)-c4cc6cccc7ccc8ccc-5c4c8c76)cc3)cc2)cc1 ; CC1(C)c2ccccc2-c2ccc(N(c3cccc(-c4ccccc4)c3)c3ccc4c5c(cccc35)-c3sc(-c5ccccc5)nc3-4)cc21 ; CC1(C)c2ccccc2-c2ccc(N(c3ccccc3-c3ccccc3)c3ccc4c5c(cccc35)-c3sc(-c5ccccc5)nc3-4)cc21]

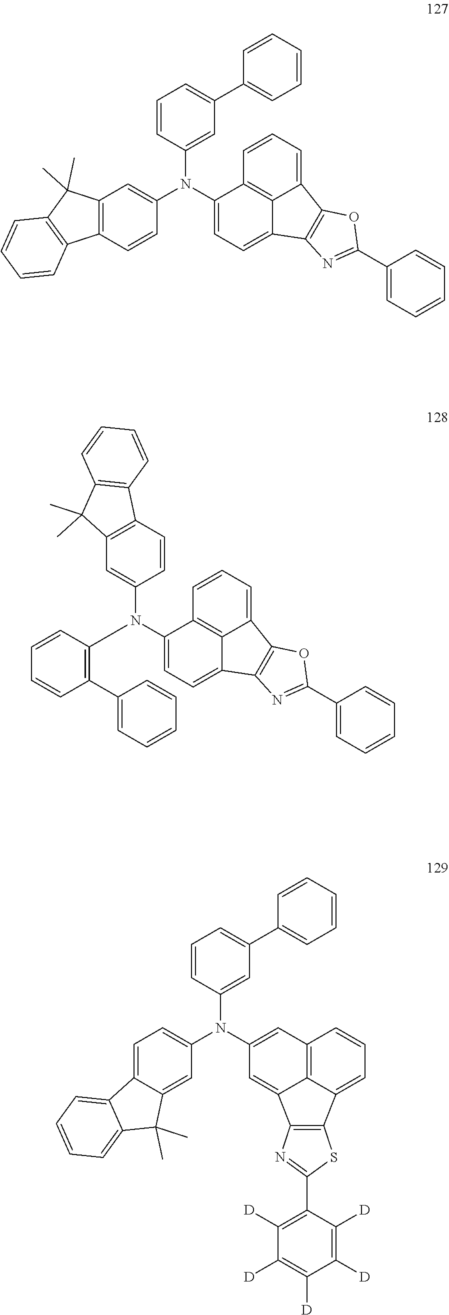 [SMILES: CC1(C)c2ccccc2-c2ccc(N(c3cccc(-c4ccccc4)c3)c3ccc4c5c(cccc35)-c3oc(-c5ccccc5)nc3-4)cc21 ; CC1(C)c2ccccc2-c2ccc(N(c3ccccc3-c3ccccc3)c3ccc4c5c(cccc35)-c3oc(-c5ccccc5)nc3-4)cc21 ; [2H]c1c([2H])c([2H])c(-c2nc3c(s2)-c2cccc4cc(N(c5cccc(-c6ccccc6)c5)c5ccc6c(c5)C(C)(C)c5ccccc5-6)cc-3c24)c([2H])c1[2H]]

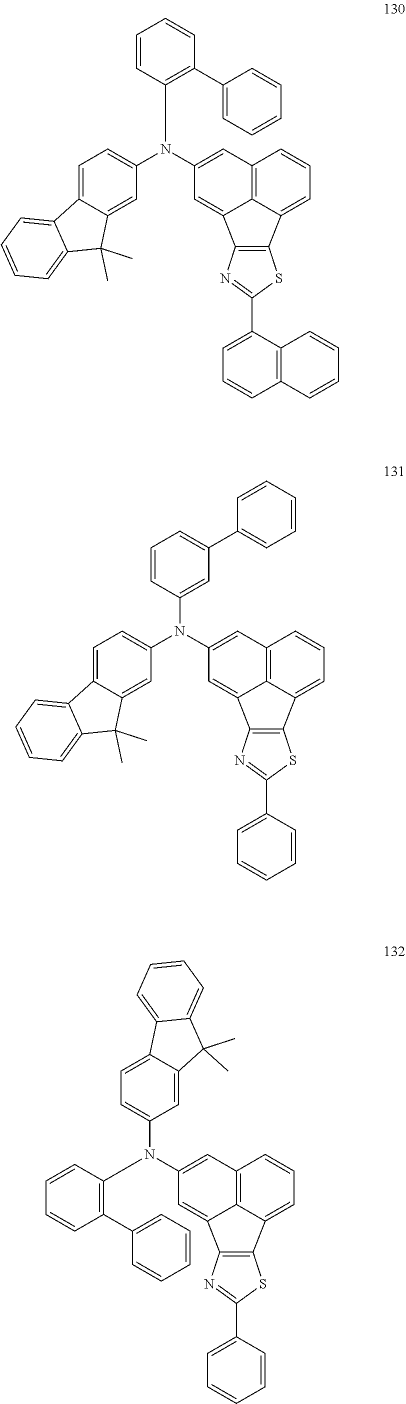 [SMILES: CC1(C)c2ccccc2-c2ccc(N(c3cc4c5c(cccc5c3)-c3sc(-c5cccc6ccccc56)nc3-4)c3ccccc3-c3ccccc3)cc21 ; CC1(C)c2ccccc2-c2ccc(N(c3cccc(-c4ccccc4)c3)c3cc4c5c(cccc5c3)-c3sc(-c5ccccc5)nc3-4)cc21 ; CC1(C)c2ccccc2-c2ccc(N(c3cc4c5c(cccc5c3)-c3sc(-c5ccccc5)nc3-4)c3ccccc3-c3ccccc3)cc21]

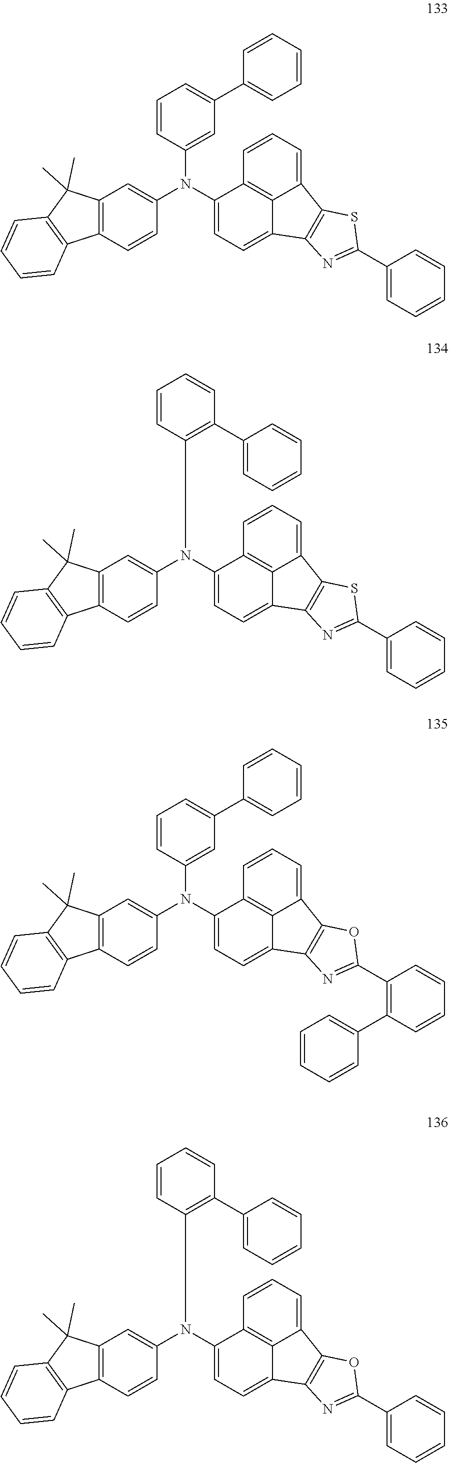 [SMILES: CC1(C)c2ccccc2-c2ccc(N(c3cccc(-c4ccccc4)c3)c3ccc4c5c(cccc35)-c3sc(-c5ccccc5)nc3-4)cc21 ; CC1(C)c2ccccc2-c2ccc(N(c3ccccc3-c3ccccc3)c3ccc4c5c(cccc35)-c3sc(-c5ccccc5)nc3-4)cc21 ; CC1(C)c2ccccc2-c2ccc(N(c3cccc(-c4ccccc4)c3)c3ccc4c5c(cccc35)-c3oc(-c5ccccc5-c5ccccc5)nc3-4)cc21 ; CC1(C)c2ccccc2-c2ccc(N(c3ccccc3-c3ccccc3)c3ccc4c5c(cccc35)-c3oc(-c5ccccc5)nc3-4)cc21]

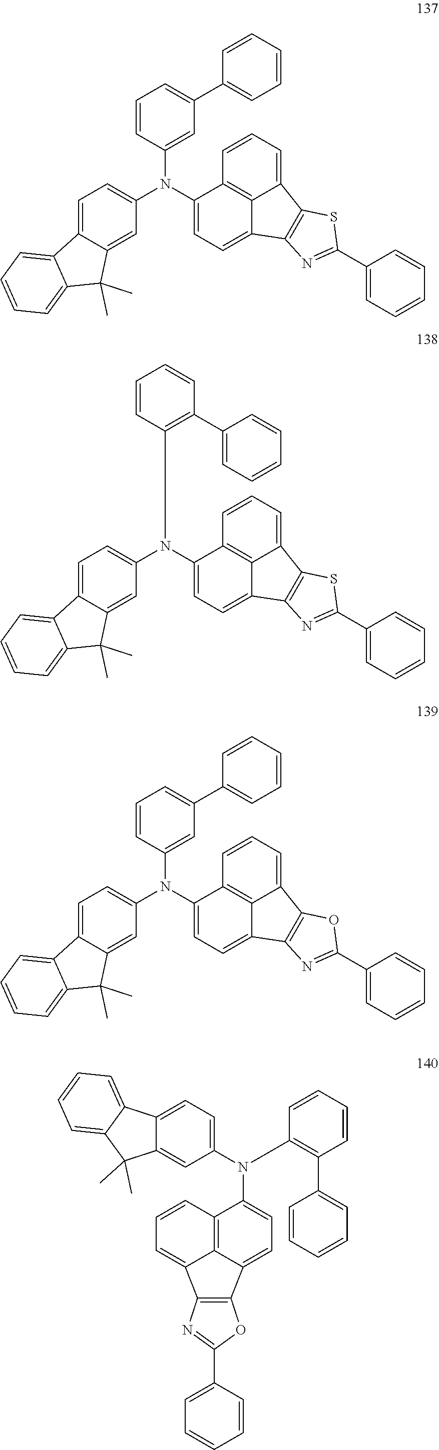 [SMILES: CC1(C)c2ccccc2-c2ccc(N(c3cccc(-c4ccccc4)c3)c3ccc4c5c(cccc35)-c3sc(-c5ccccc5)nc3-4)cc21 ; CC1(C)c2ccccc2-c2ccc(N(c3ccccc3-c3ccccc3)c3ccc4c5c(cccc35)-c3sc(-c5ccccc5)nc3-4)cc21 ; CC1(C)c2ccccc2-c2ccc(N(c3cccc(-c4ccccc4)c3)c3ccc4c5c(cccc35)-c3oc(-c5ccccc5)nc3-4)cc21 ; CC1(C)c2ccccc2-c2ccc(N(c3ccccc3-c3ccccc3)c3ccc4c5c(cccc35)-c3nc(-c5ccccc5)oc3-4)cc21]

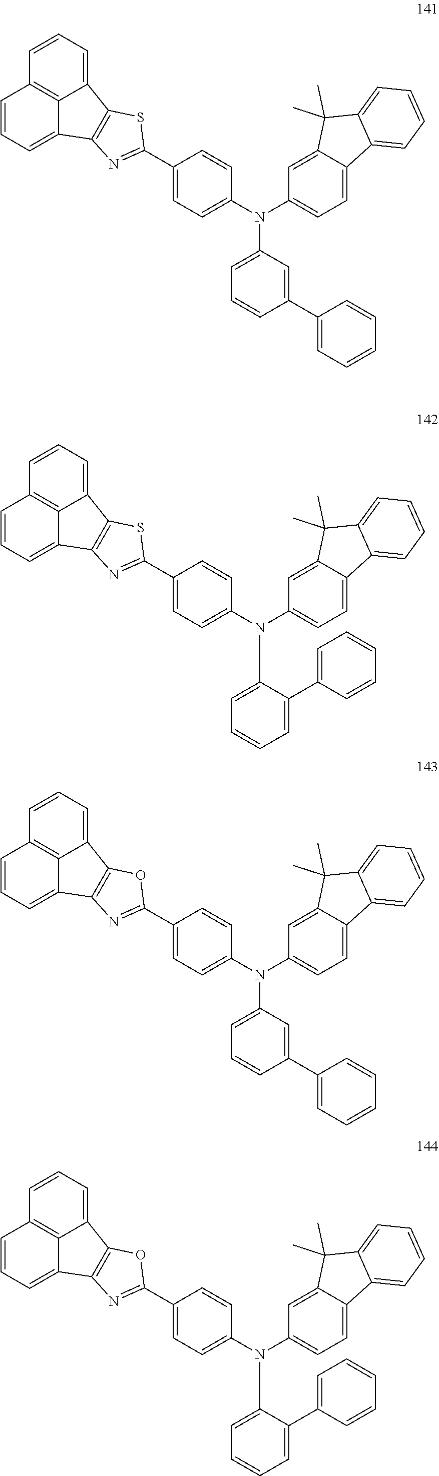 [SMILES: CC1(C)c2ccccc2-c2ccc(N(c3ccc(-c4nc5c(s4)-c4cccc6cccc-5c46)cc3)c3cccc(-c4ccccc4)c3)cc21 ; CC1(C)c2ccccc2-c2ccc(N(c3ccc(-c4nc5c(s4)-c4cccc6cccc-5c46)cc3)c3ccccc3-c3ccccc3)cc21 ; CC1(C)c2ccccc2-c2ccc(N(c3ccc(-c4nc5c(o4)-c4cccc6cccc-5c46)cc3)c3cccc(-c4ccccc4)c3)cc21 ; CC1(C)c2ccccc2-c2ccc(N(c3ccc(-c4nc5c(o4)-c4cccc6cccc-5c46)cc3)c3ccccc3-c3ccccc3)cc21]

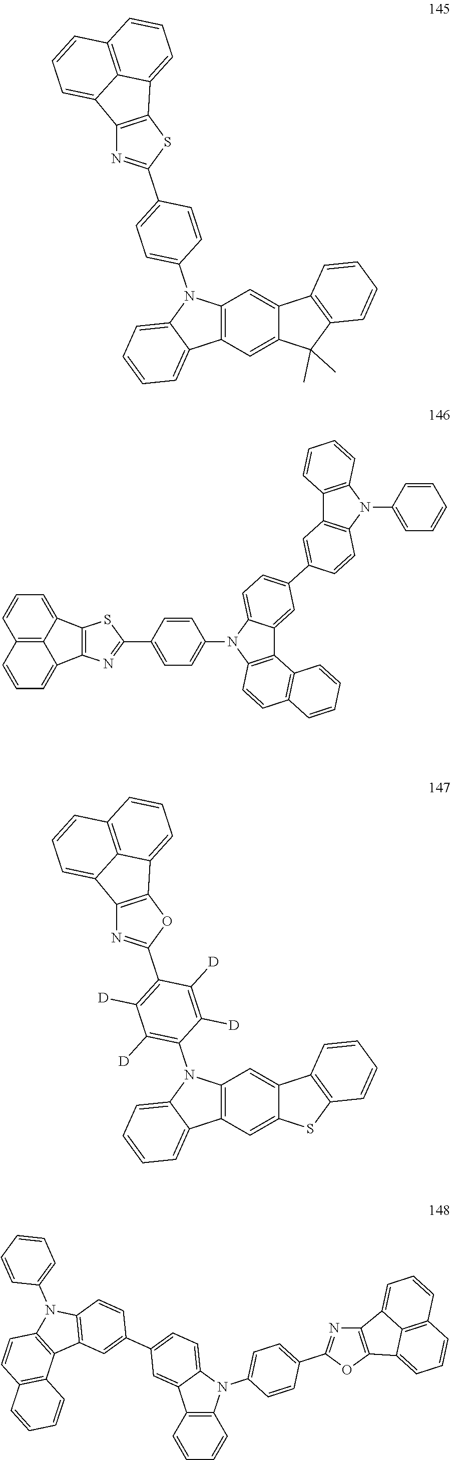 [SMILES: CC1(C)c2ccccc2-c2cc3c(cc21)c1ccccc1n3-c1ccc(-c2nc3c(s2)-c2cccc4cccc-3c24)cc1 ; c1ccc(-n2c3ccccc3c3cc(-c4ccc5c(c4)c4c6ccccc6ccc4n5-c4ccc(-c5nc6c(s5)-c5cccc7cccc-6c57)cc4)ccc32)cc1 ; [2H]c1c([2H])c(-n2c3ccccc3c3cc4sc5ccccc5c4cc32)c([2H])c([2H])c1-c1nc2c(o1)-c1cccc3cccc-2c13 ; c1ccc(-n2c3ccc(-c4ccc5c(c4)c4ccccc4n5-c4ccc(-c5nc6c(o5)-c5cccc7cccc-6c57)cc4)cc3c3c4ccccc4ccc32)cc1]

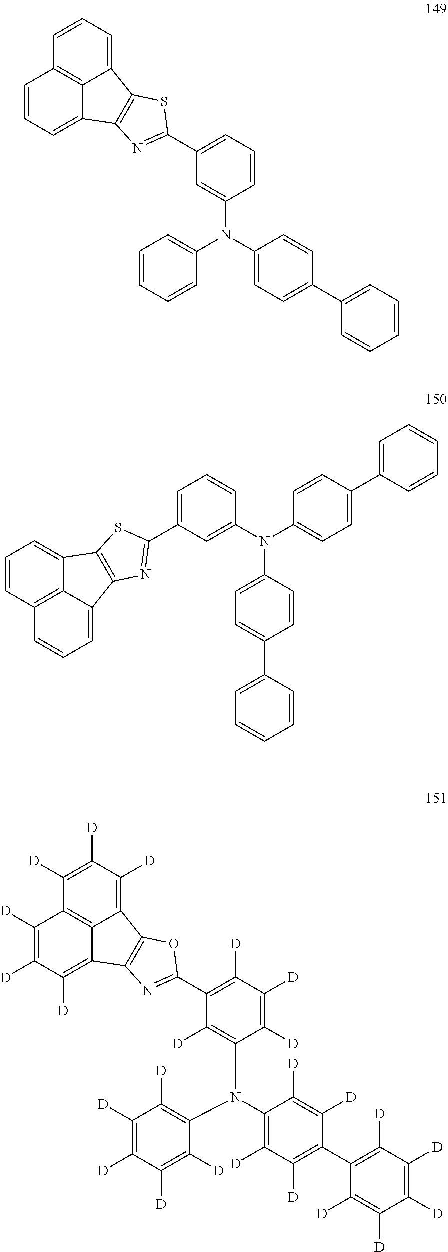 [SMILES: c1ccc(-c2ccc(N(c3ccccc3)c3cccc(-c4nc5c(s4)-c4cccc6cccc-5c46)c3)cc2)cc1 ; c1ccc(-c2ccc(N(c3ccc(-c4ccccc4)cc3)c3cccc(-c4nc5c(s4)-c4cccc6cccc-5c46)c3)cc2)cc1 ; [2H]c1c([2H])c([2H])c(-c2c([2H])c([2H])c(N(c3c([2H])c([2H])c([2H])c([2H])c3[2H])c3c([2H])c([2H])c([2H])c(-c4nc5c(o4)-c4c([2H])c([2H])c([2H])c6c([2H])c([2H])c([2H])c-5c46)c3[2H])c([2H])c2[2H])c([2H])c1[2H]]

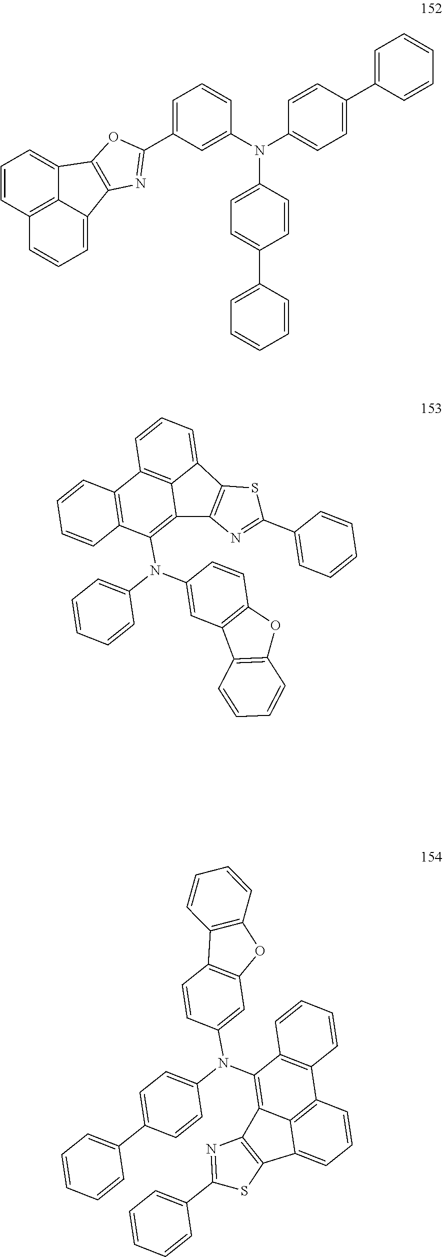 [SMILES: c1ccc(-c2ccc(N(c3ccc(-c4ccccc4)cc3)c3cccc(-c4nc5c(o4)-c4cccc6cccc-5c46)c3)cc2)cc1 ; c1ccc(-c2nc3c(s2)-c2cccc4c2c-3c(N(c2ccccc2)c2ccc3oc5ccccc5c3c2)c2ccccc24)cc1 ; c1ccc(-c2ccc(N(c3ccc4c(c3)oc3ccccc34)c3c4c5c(cccc5c5ccccc35)-c3sc(-c5ccccc5)nc3-4)cc2)cc1]

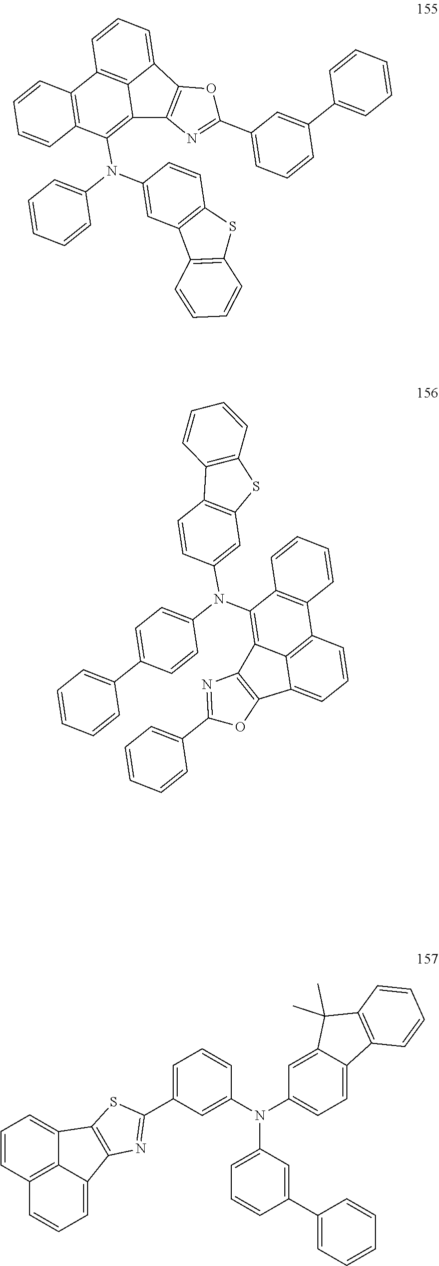 [SMILES: c1ccc(-c2cccc(-c3nc4c(o3)-c3cccc5c3c-4c(N(c3ccccc3)c3ccc4sc6ccccc6c4c3)c3ccccc35)c2)cc1 ; c1ccc(-c2ccc(N(c3ccc4c(c3)sc3ccccc34)c3c4c5c(cccc5c5ccccc35)-c3oc(-c5ccccc5)nc3-4)cc2)cc1 ; CC1(C)c2ccccc2-c2ccc(N(c3cccc(-c4ccccc4)c3)c3cccc(-c4nc5c(s4)-c4cccc6cccc-5c46)c3)cc21]

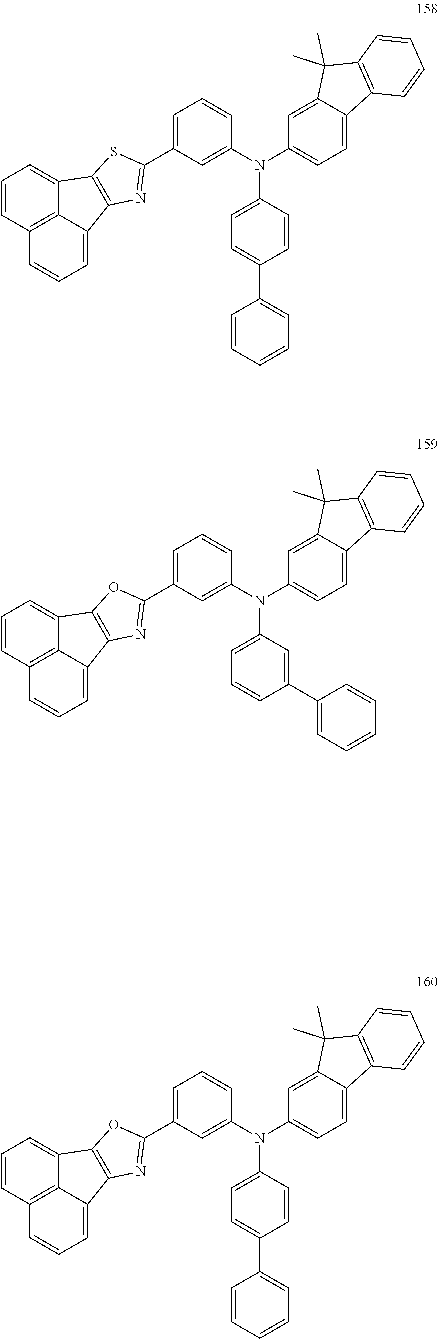 [SMILES: CC1(C)c2ccccc2-c2ccc(N(c3ccc(-c4ccccc4)cc3)c3cccc(-c4nc5c(s4)-c4cccc6cccc-5c46)c3)cc21 ; CC1(C)c2ccccc2-c2ccc(N(c3cccc(-c4ccccc4)c3)c3cccc(-c4nc5c(o4)-c4cccc6cccc-5c46)c3)cc21 ; CC1(C)c2ccccc2-c2ccc(N(c3ccc(-c4ccccc4)cc3)c3cccc(-c4nc5c(o4)-c4cccc6cccc-5c46)c3)cc21]

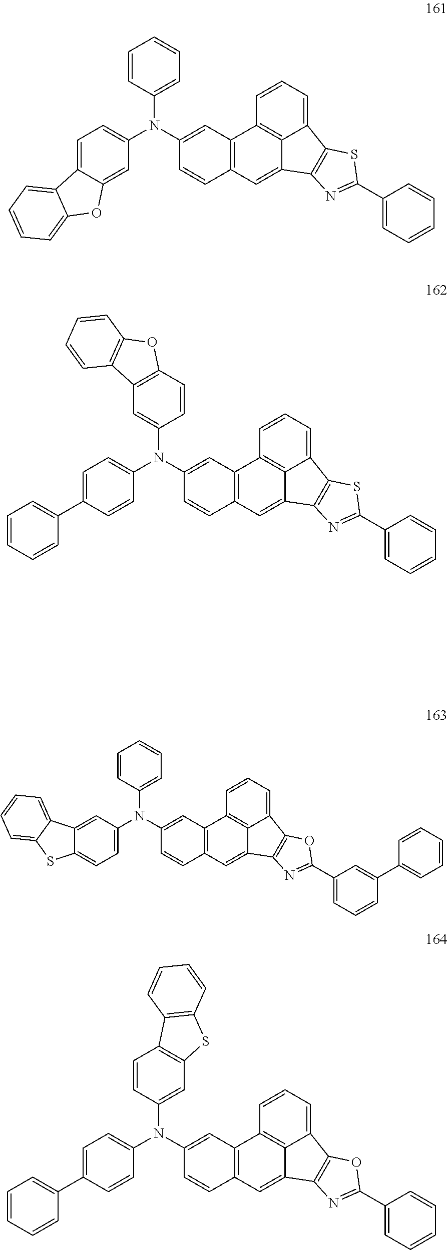

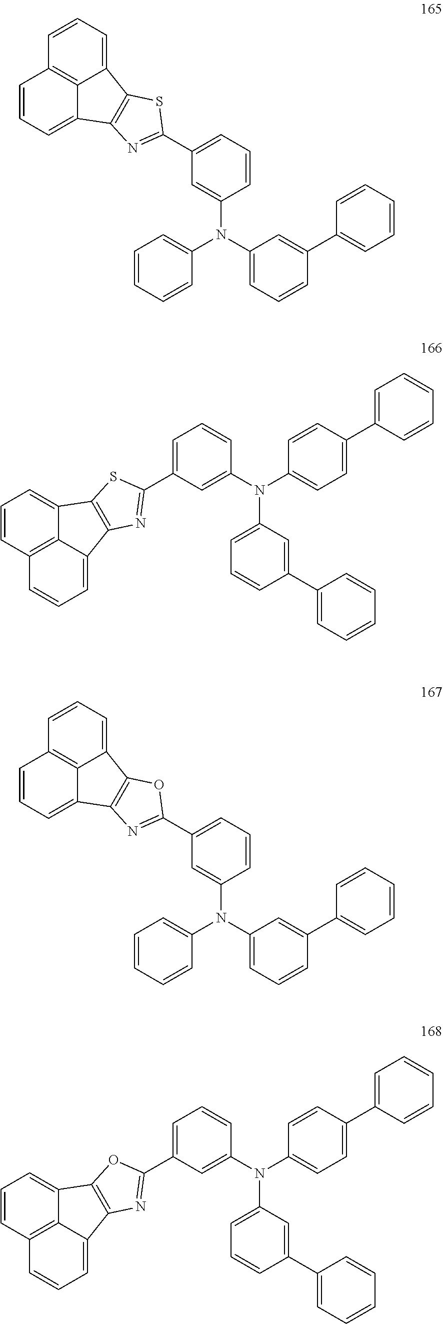 [SMILES: c1ccc(-c2cccc(N(c3ccccc3)c3cccc(-c4nc5c(s4)-c4cccc6cccc-5c46)c3)c2)cc1 ; c1ccc(-c2ccc(N(c3cccc(-c4ccccc4)c3)c3cccc(-c4nc5c(s4)-c4cccc6cccc-5c46)c3)cc2)cc1 ; c1ccc(-c2cccc(N(c3ccccc3)c3cccc(-c4nc5c(o4)-c4cccc6cccc-5c46)c3)c2)cc1 ; c1ccc(-c2ccc(N(c3cccc(-c4ccccc4)c3)c3cccc(-c4nc5c(o4)-c4cccc6cccc-5c46)c3)cc2)cc1]

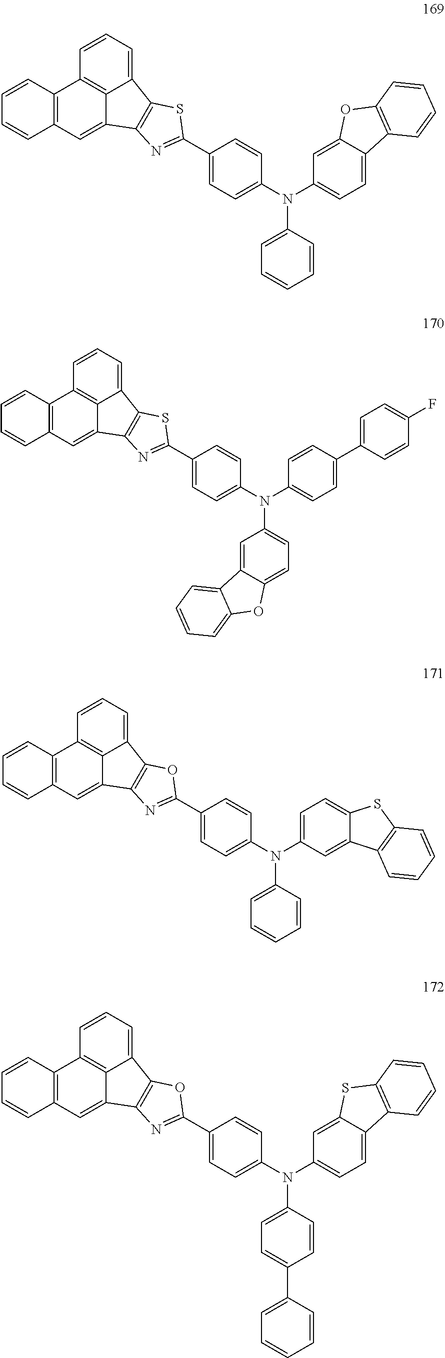 [SMILES: c1ccc(N(c2ccc(-c3nc4c(s3)-c3cccc5c3c-4cc3ccccc35)cc2)c2ccc3c(c2)oc2ccccc23)cc1 ; Fc1ccc(-c2ccc(N(c3ccc(-c4nc5c(s4)-c4cccc6c4c-5cc4ccccc46)cc3)c3ccc4oc5ccccc5c4c3)cc2)cc1 ; c1ccc(N(c2ccc(-c3nc4c(o3)-c3cccc5c3c-4cc3ccccc35)cc2)c2ccc3sc4ccccc4c3c2)cc1 ; c1ccc(-c2ccc(N(c3ccc(-c4nc5c(o4)-c4cccc6c4c-5cc4ccccc46)cc3)c3ccc4c(c3)sc3ccccc34)cc2)cc1]

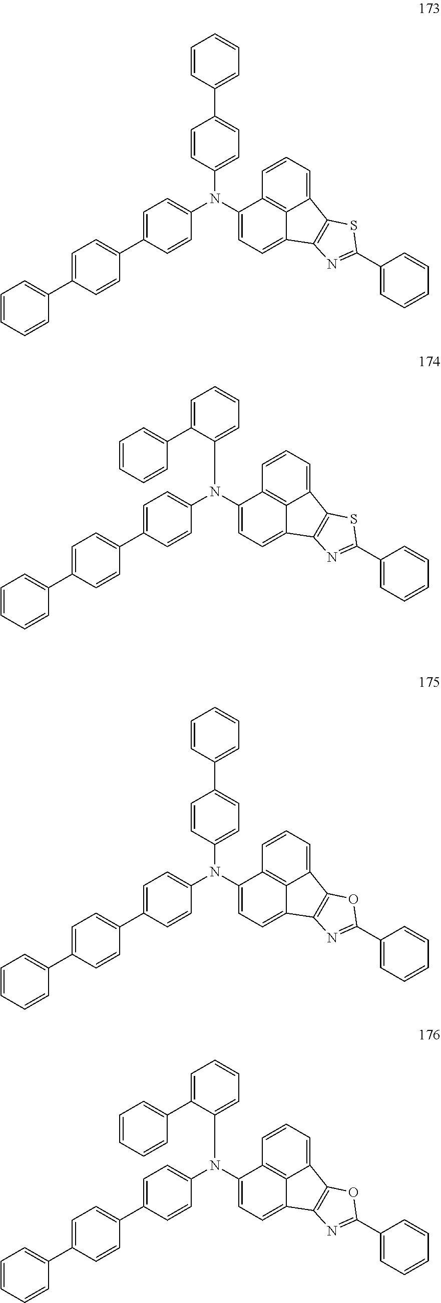 [SMILES: c1ccc(-c2ccc(-c3ccc(N(c4ccc(-c5ccccc5)cc4)c4ccc5c6c(cccc46)-c4sc(-c6ccccc6)nc4-5)cc3)cc2)cc1 ; c1ccc(-c2ccc(-c3ccc(N(c4ccccc4-c4ccccc4)c4ccc5c6c(cccc46)-c4sc(-c6ccccc6)nc4-5)cc3)cc2)cc1 ; c1ccc(-c2ccc(-c3ccc(N(c4ccc(-c5ccccc5)cc4)c4ccc5c6c(cccc46)-c4oc(-c6ccccc6)nc4-5)cc3)cc2)cc1 ; c1ccc(-c2ccc(-c3ccc(N(c4ccccc4-c4ccccc4)c4ccc5c6c(cccc46)-c4oc(-c6ccccc6)nc4-5)cc3)cc2)cc1]

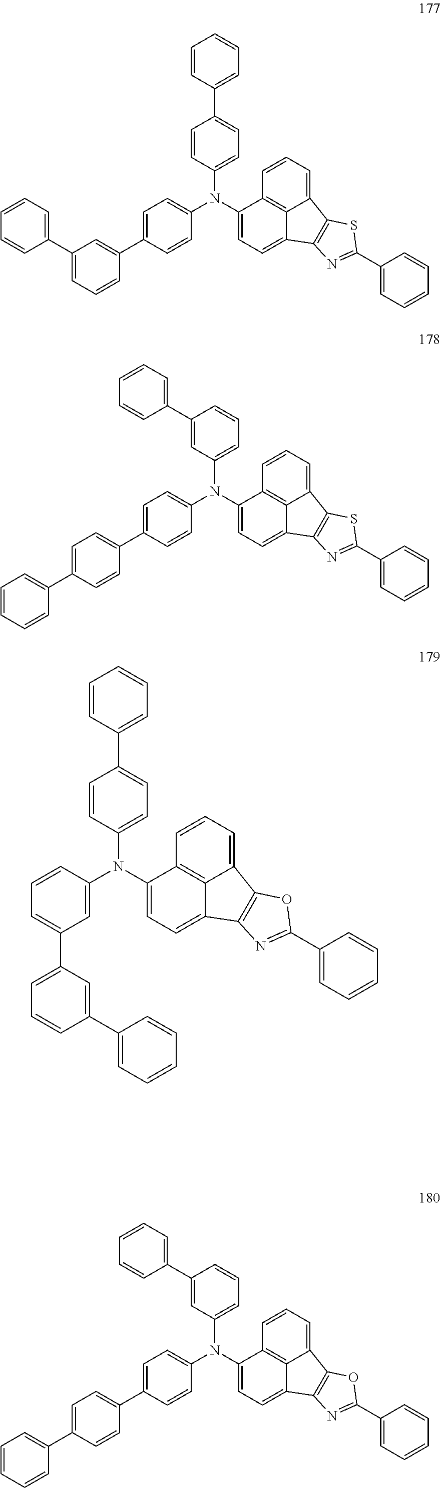 [SMILES: c1ccc(-c2ccc(N(c3ccc(-c4cccc(-c5ccccc5)c4)cc3)c3ccc4c5c(cccc35)-c3sc(-c5ccccc5)nc3-4)cc2)cc1 ; c1ccc(-c2ccc(-c3ccc(N(c4cccc(-c5ccccc5)c4)c4ccc5c6c(cccc46)-c4sc(-c6ccccc6)nc4-5)cc3)cc2)cc1 ; c1ccc(-c2ccc(N(c3cccc(-c4cccc(-c5ccccc5)c4)c3)c3ccc4c5c(cccc35)-c3oc(-c5ccccc5)nc3-4)cc2)cc1 ; c1ccc(-c2ccc(-c3ccc(N(c4cccc(-c5ccccc5)c4)c4ccc5c6c(cccc46)-c4oc(-c6ccccc6)nc4-5)cc3)cc2)cc1]

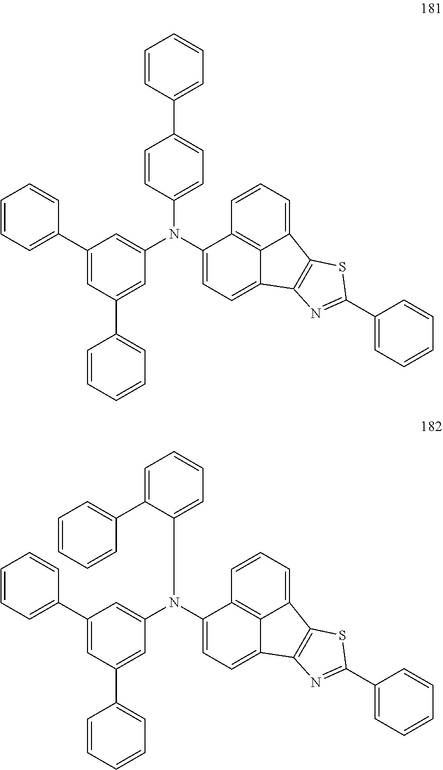 [SMILES: c1ccc(-c2ccc(N(c3cc(-c4ccccc4)cc(-c4ccccc4)c3)c3ccc4c5c(cccc35)-c3sc(-c5ccccc5)nc3-4)cc2)cc1 ; c1ccc(-c2cc(-c3ccccc3)cc(N(c3ccccc3-c3ccccc3)c3ccc4c5c(cccc35)-c3sc(-c5ccccc5)nc3-4)c2)cc1]

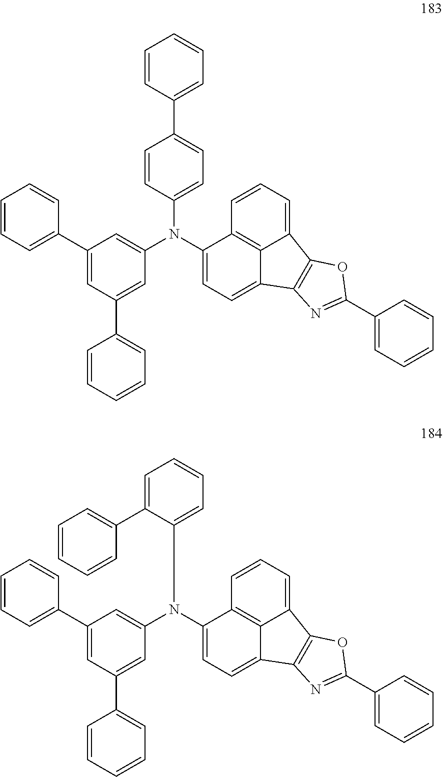 [SMILES: c1ccc(-c2ccc(N(c3cc(-c4ccccc4)cc(-c4ccccc4)c3)c3ccc4c5c(cccc35)-c3oc(-c5ccccc5)nc3-4)cc2)cc1 ; c1ccc(-c2cc(-c3ccccc3)cc(N(c3ccccc3-c3ccccc3)c3ccc4c5c(cccc35)-c3oc(-c5ccccc5)nc3-4)c2)cc1]

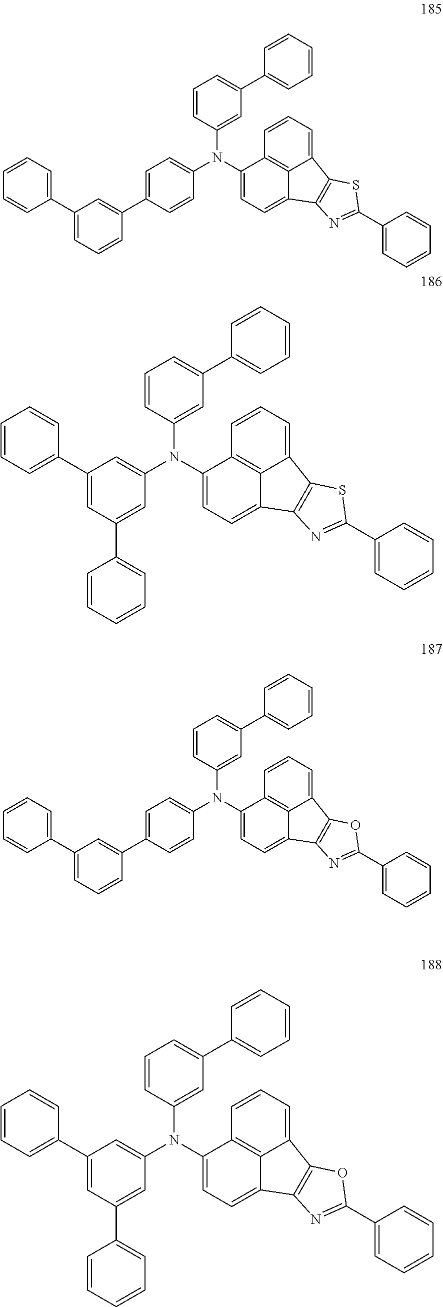 [SMILES: c1ccc(-c2cccc(-c3ccc(N(c4cccc(-c5ccccc5)c4)c4ccc5c6c(cccc46)-c4sc(-c6ccccc6)nc4-5)cc3)c2)cc1 ; c1ccc(-c2cccc(N(c3cc(-c4ccccc4)cc(-c4ccccc4)c3)c3ccc4c5c(cccc35)-c3sc(-c5ccccc5)nc3-4)c2)cc1 ; c1ccc(-c2cccc(-c3ccc(N(c4cccc(-c5ccccc5)c4)c4ccc5c6c(cccc46)-c4oc(-c6ccccc6)nc4-5)cc3)c2)cc1 ; c1ccc(-c2cccc(N(c3cc(-c4ccccc4)cc(-c4ccccc4)c3)c3ccc4c5c(cccc35)-c3oc(-c5ccccc5)nc3-4)c2)cc1]

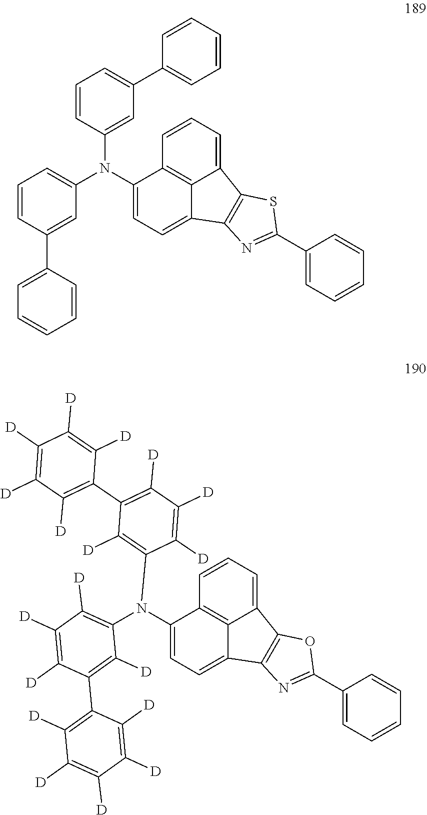 [SMILES: c1ccc(-c2cccc(N(c3cccc(-c4ccccc4)c3)c3ccc4c5c(cccc35)-c3sc(-c5ccccc5)nc3-4)c2)cc1 ; [2H]c1c([2H])c([2H])c(-c2c([2H])c([2H])c([2H])c(N(c3c([2H])c([2H])c([2H])c(-c4c([2H])c([2H])c([2H])c([2H])c4[2H])c3[2H])c3ccc4c5c(cccc35)-c3oc(-c5ccccc5)nc3-4)c2[2H])c([2H])c1[2H]]

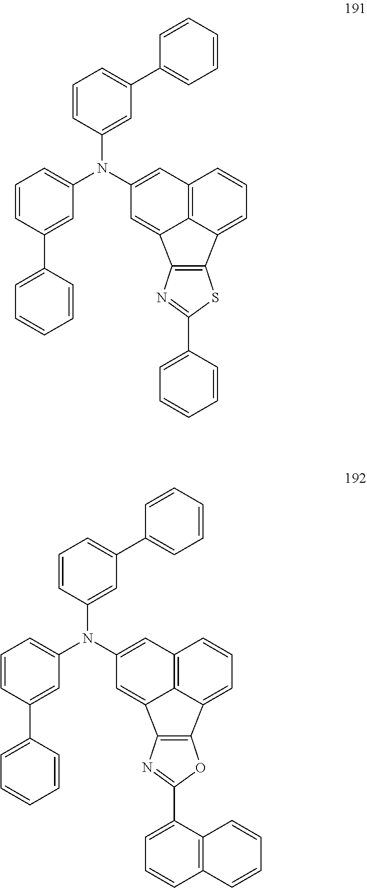 [SMILES: c1ccc(-c2cccc(N(c3cccc(-c4ccccc4)c3)c3cc4c5c(cccc5c3)-c3sc(-c5ccccc5)nc3-4)c2)cc1 ; c1ccc(-c2cccc(N(c3cccc(-c4ccccc4)c3)c3cc4c5c(cccc5c3)-c3oc(-c5cccc6ccccc56)nc3-4)c2)cc1]

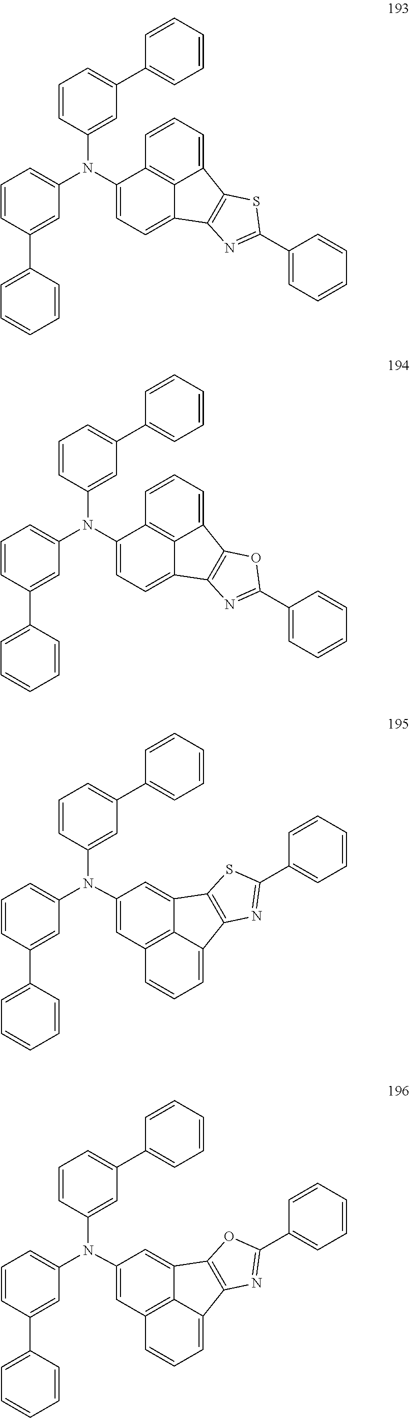 [SMILES: c1ccc(-c2cccc(N(c3cccc(-c4ccccc4)c3)c3ccc4c5c(cccc35)-c3sc(-c5ccccc5)nc3-4)c2)cc1 ; c1ccc(-c2cccc(N(c3cccc(-c4ccccc4)c3)c3ccc4c5c(cccc35)-c3oc(-c5ccccc5)nc3-4)c2)cc1 ; c1ccc(-c2cccc(N(c3cccc(-c4ccccc4)c3)c3cc4c5c(cccc5c3)-c3nc(-c5ccccc5)sc3-4)c2)cc1 ; c1ccc(-c2cccc(N(c3cccc(-c4ccccc4)c3)c3cc4c5c(cccc5c3)-c3nc(-c5ccccc5)oc3-4)c2)cc1]

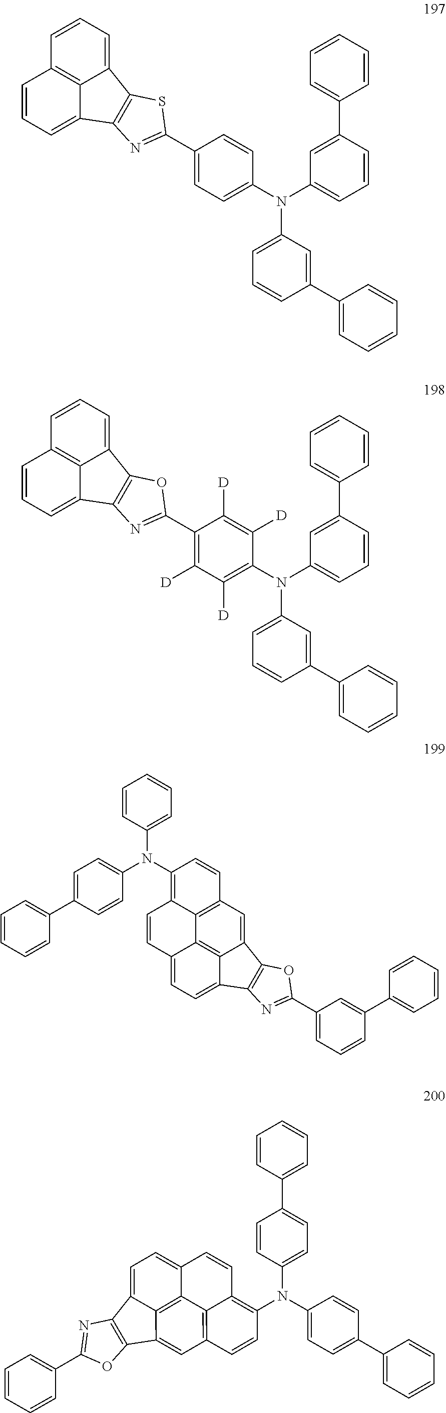 [SMILES: c1ccc(-c2cccc(N(c3ccc(-c4nc5c(s4)-c4cccc6cccc-5c46)cc3)c3cccc(-c4ccccc4)c3)c2)cc1 ; [2H]c1c([2H])c(N(c2cccc(-c3ccccc3)c2)c2cccc(-c3ccccc3)c2)c([2H])c([2H])c1-c1nc2c(o1)-c1cccc3cccc-2c13 ; c1ccc(-c2ccc(N(c3ccccc3)c3ccc4cc5c6c(ccc7ccc3c4c76)-c3nc(-c4cccc(-c6ccccc6)c4)oc3-5)cc2)cc1 ; c1ccc(-c2ccc(N(c3ccc(-c4ccccc4)cc3)c3ccc4cc5c6c(ccc7ccc3c4c76)-c3nc(-c4ccccc4)oc3-5)cc2)cc1]

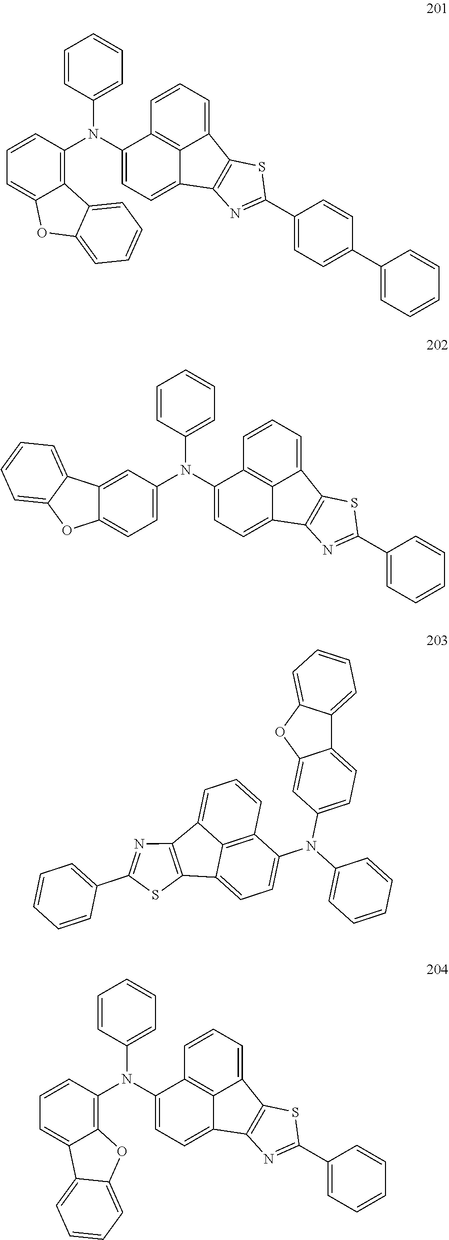 [SMILES: c1ccc(-c2ccc(-c3nc4c(s3)-c3cccc5c(N(c6ccccc6)c6cccc7oc8ccccc8c67)ccc-4c35)cc2)cc1 ; c1ccc(-c2nc3c(s2)-c2cccc4c(N(c5ccccc5)c5ccc6oc7ccccc7c6c5)ccc-3c24)cc1 ; c1ccc(-c2nc3c(s2)-c2ccc(N(c4ccccc4)c4ccc5c(c4)oc4ccccc45)c4cccc-3c24)cc1 ; c1ccc(-c2nc3c(s2)-c2cccc4c(N(c5ccccc5)c5cccc6c5oc5ccccc56)ccc-3c24)cc1]

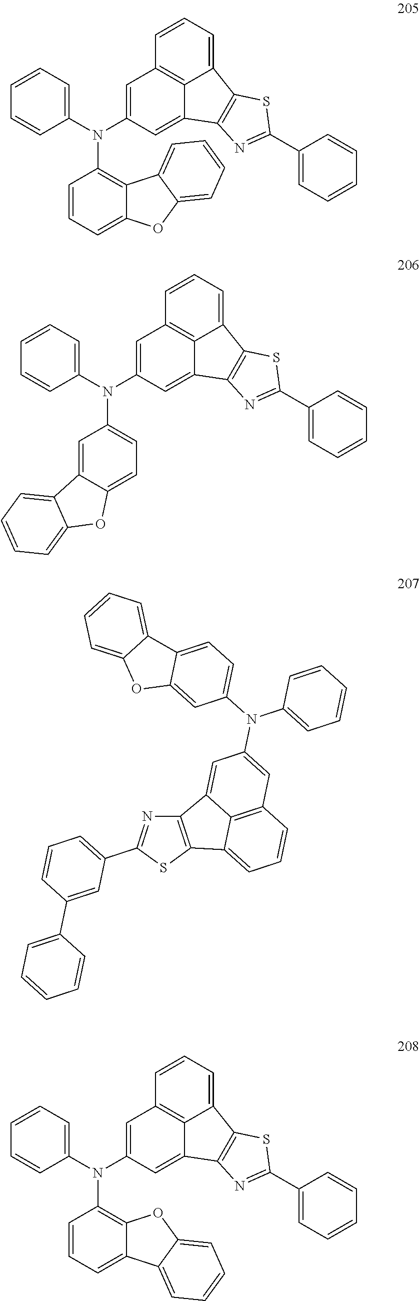 [SMILES: c1ccc(-c2nc3c(s2)-c2cccc4cc(N(c5ccccc5)c5cccc6oc7ccccc7c56)cc-3c24)cc1 ; c1ccc(-c2nc3c(s2)-c2cccc4cc(N(c5ccccc5)c5ccc6oc7ccccc7c6c5)cc-3c24)cc1 ; c1ccc(-c2cccc(-c3nc4c(s3)-c3cccc5cc(N(c6ccccc6)c6ccc7c(c6)oc6ccccc67)cc-4c35)c2)cc1 ; c1ccc(-c2nc3c(s2)-c2cccc4cc(N(c5ccccc5)c5cccc6c5oc5ccccc56)cc-3c24)cc1]

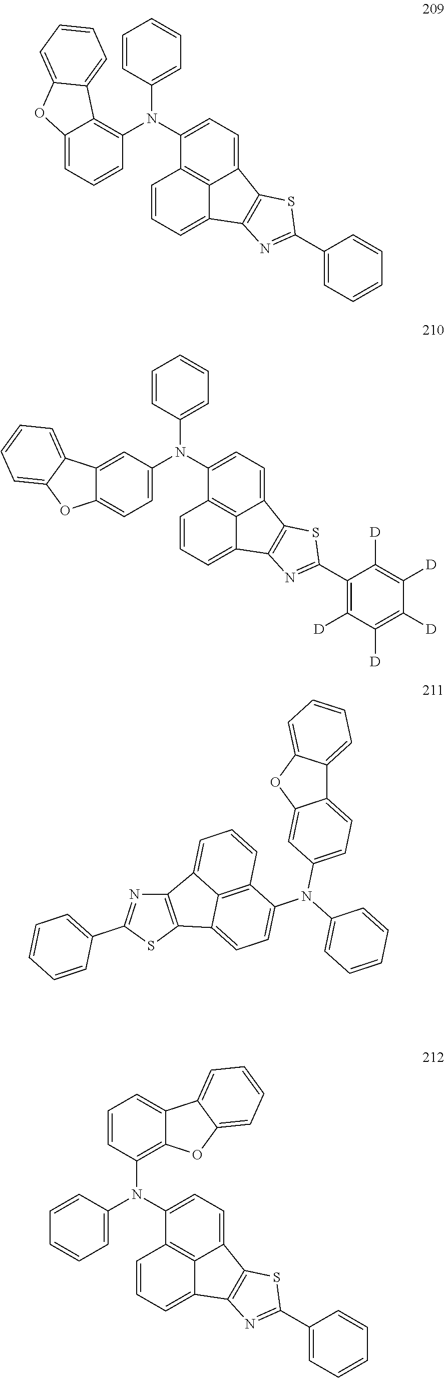 [SMILES: c1ccc(-c2nc3c(s2)-c2ccc(N(c4ccccc4)c4cccc5oc6ccccc6c45)c4cccc-3c24)cc1 ; [2H]c1c([2H])c([2H])c(-c2nc3c(s2)-c2ccc(N(c4ccccc4)c4ccc5oc6ccccc6c5c4)c4cccc-3c24)c([2H])c1[2H] ; c1ccc(-c2nc3c(s2)-c2ccc(N(c4ccccc4)c4ccc5c(c4)oc4ccccc45)c4cccc-3c24)cc1 ; c1ccc(-c2nc3c(s2)-c2ccc(N(c4ccccc4)c4cccc5c4oc4ccccc45)c4cccc-3c24)cc1]

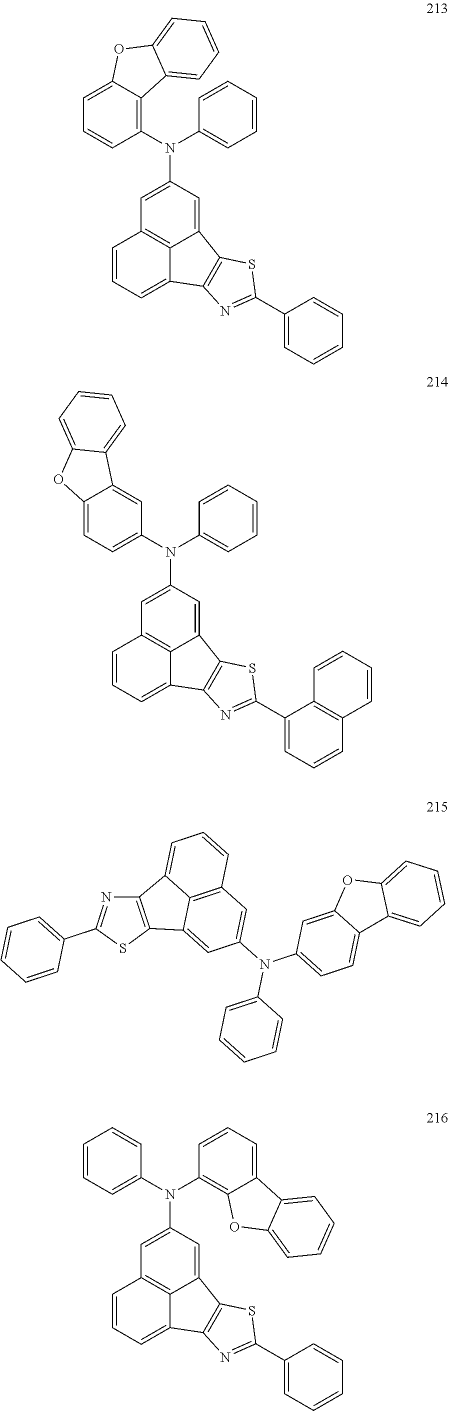 [SMILES: c1ccc(-c2nc3c(s2)-c2cc(N(c4ccccc4)c4cccc5oc6ccccc6c45)cc4cccc-3c24)cc1 ; c1ccc(N(c2cc3c4c(cccc4c2)-c2nc(-c4cccc5ccccc45)sc2-3)c2ccc3oc4ccccc4c3c2)cc1 ; c1ccc(-c2nc3c(s2)-c2cc(N(c4ccccc4)c4ccc5c(c4)oc4ccccc45)cc4cccc-3c24)cc1 ; c1ccc(-c2nc3c(s2)-c2cc(N(c4ccccc4)c4cccc5c4oc4ccccc45)cc4cccc-3c24)cc1]

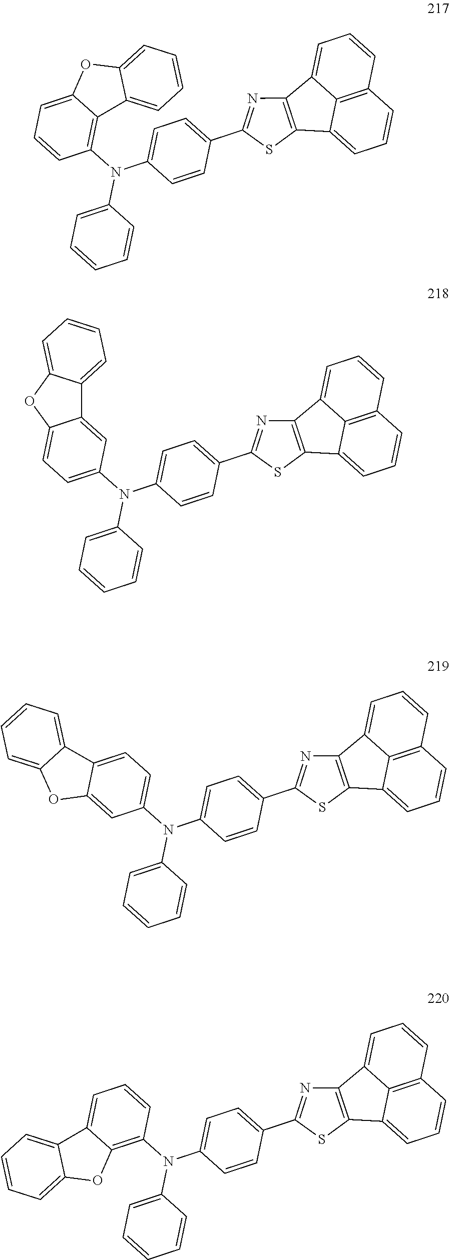 [SMILES: c1ccc(N(c2ccc(-c3nc4c(s3)-c3cccc5cccc-4c35)cc2)c2cccc3oc4ccccc4c23)cc1 ; c1ccc(N(c2ccc(-c3nc4c(s3)-c3cccc5cccc-4c35)cc2)c2ccc3oc4ccccc4c3c2)cc1 ; c1ccc(N(c2ccc(-c3nc4c(s3)-c3cccc5cccc-4c35)cc2)c2ccc3c(c2)oc2ccccc23)cc1 ; c1ccc(N(c2ccc(-c3nc4c(s3)-c3cccc5cccc-4c35)cc2)c2cccc3c2oc2ccccc23)cc1]

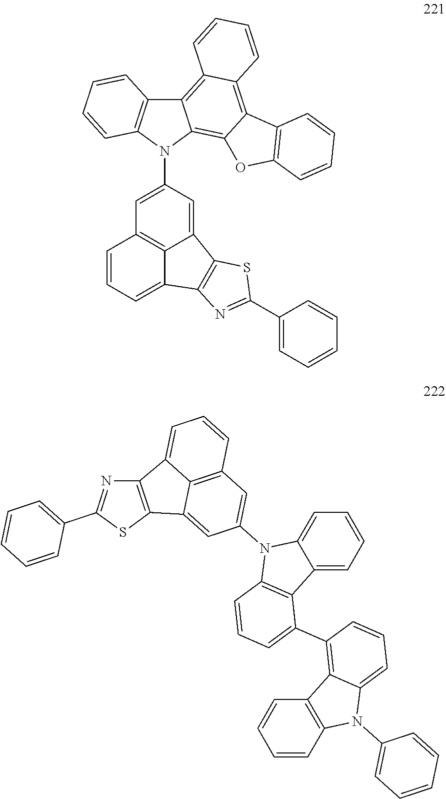 [SMILES: c1ccc(-c2nc3c(s2)-c2cc(-n4c5ccccc5c5c6ccccc6c6c7ccccc7oc6c54)cc4cccc-3c24)cc1 ; c1ccc(-c2nc3c(s2)-c2cc(-n4c5ccccc5c5c(-c6cccc7c6c6ccccc6n7-c6ccccc6)cccc54)cc4cccc-3c24)cc1]

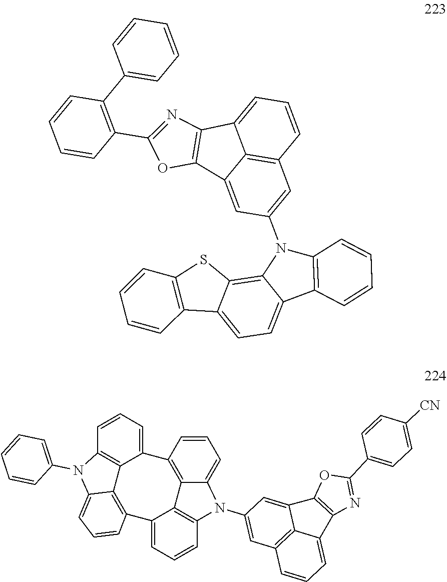 [SMILES: c1ccc(-c2ccccc2-c2nc3c(o2)-c2cc(-n4c5ccccc5c5ccc6c7ccccc7sc6c54)cc4cccc-3c24)cc1 ; N#Cc1ccc(-c2nc3c(o2)-c2cc(-n4c5cccc6c7cccc8c7c7c(cccc7n8-c7ccccc7)c7cccc4c7c65)cc4cccc-3c24)cc1]

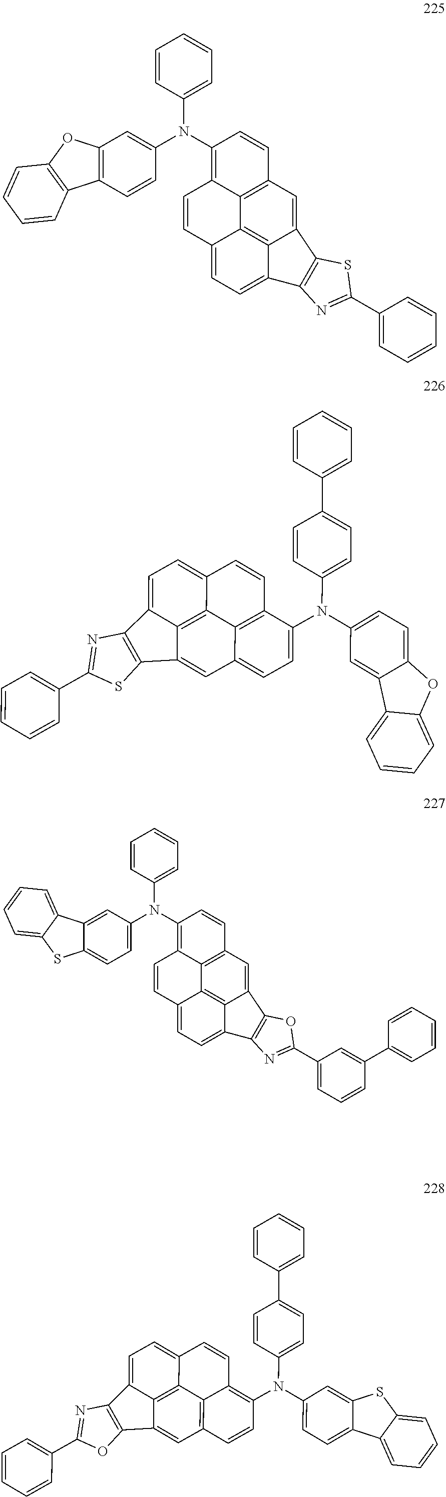 [SMILES: c1ccc(-c2nc3c(s2)-c2cc4ccc(N(c5ccccc5)c5ccc6c(c5)oc5ccccc56)c5ccc6ccc-3c2c6c45)cc1 ; c1ccc(-c2ccc(N(c3ccc4oc5ccccc5c4c3)c3ccc4cc5c6c(ccc7ccc3c4c76)-c3nc(-c4ccccc4)sc3-5)cc2)cc1 ; c1ccc(-c2cccc(-c3nc4c(o3)-c3cc5ccc(N(c6ccccc6)c6ccc7sc8ccccc8c7c6)c6ccc7ccc-4c3c7c56)c2)cc1 ; c1ccc(-c2ccc(N(c3ccc4c(c3)sc3ccccc34)c3ccc4cc5c6c(ccc7ccc3c4c76)-c3nc(-c4ccccc4)oc3-5)cc2)cc1]

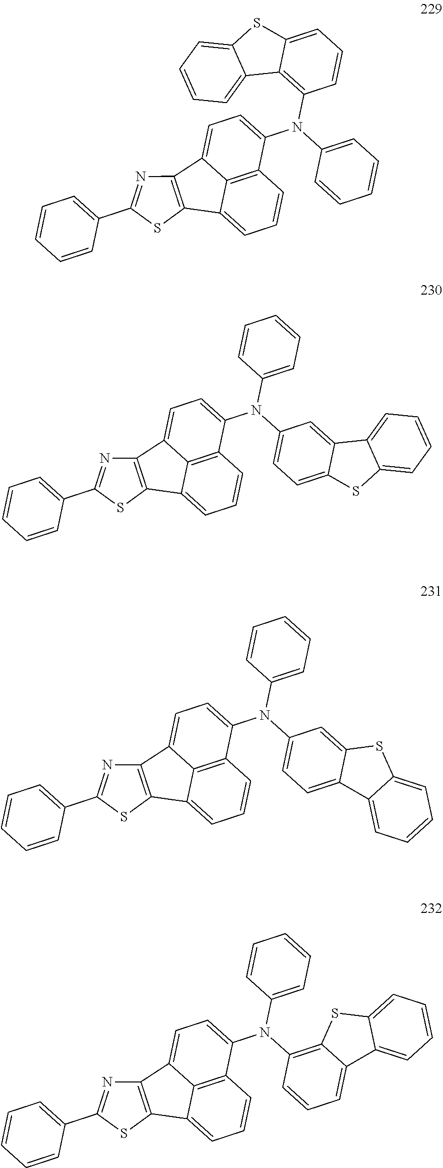 [SMILES: c1ccc(-c2nc3c(s2)-c2cccc4c(N(c5ccccc5)c5cccc6sc7ccccc7c56)ccc-3c24)cc1 ; c1ccc(-c2nc3c(s2)-c2cccc4c(N(c5ccccc5)c5ccc6sc7ccccc7c6c5)ccc-3c24)cc1 ; c1ccc(-c2nc3c(s2)-c2cccc4c(N(c5ccccc5)c5ccc6c(c5)sc5ccccc56)ccc-3c24)cc1 ; c1ccc(-c2nc3c(s2)-c2cccc4c(N(c5ccccc5)c5cccc6c5sc5ccccc56)ccc-3c24)cc1]

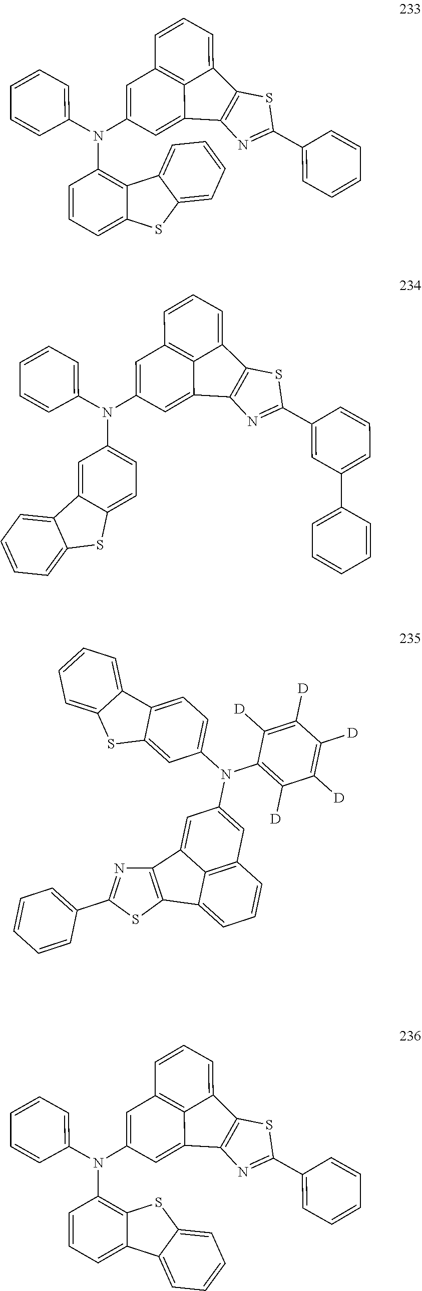 [SMILES: c1ccc(-c2nc3c(s2)-c2cccc4cc(N(c5ccccc5)c5cccc6sc7ccccc7c56)cc-3c24)cc1 ; c1ccc(-c2cccc(-c3nc4c(s3)-c3cccc5cc(N(c6ccccc6)c6ccc7sc8ccccc8c7c6)cc-4c35)c2)cc1 ; [2H]c1c([2H])c([2H])c(N(c2cc3c4c(cccc4c2)-c2sc(-c4ccccc4)nc2-3)c2ccc3c(c2)sc2ccccc23)c([2H])c1[2H] ; c1ccc(-c2nc3c(s2)-c2cccc4cc(N(c5ccccc5)c5cccc6c5sc5ccccc56)cc-3c24)cc1]

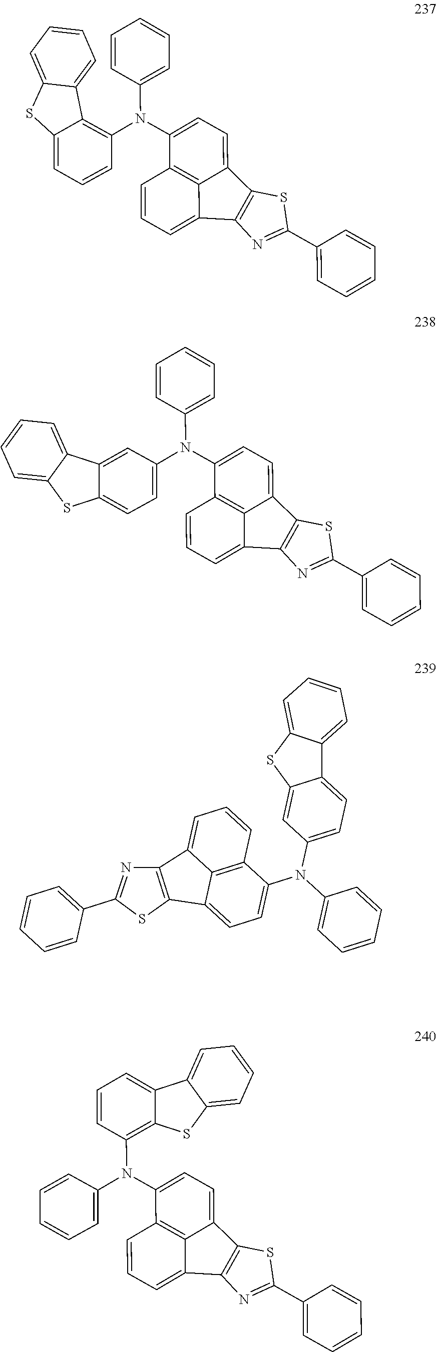 [SMILES: c1ccc(-c2nc3c(s2)-c2ccc(N(c4ccccc4)c4cccc5sc6ccccc6c45)c4cccc-3c24)cc1 ; c1ccc(-c2nc3c(s2)-c2ccc(N(c4ccccc4)c4ccc5sc6ccccc6c5c4)c4cccc-3c24)cc1 ; c1ccc(-c2nc3c(s2)-c2ccc(N(c4ccccc4)c4ccc5c(c4)sc4ccccc45)c4cccc-3c24)cc1 ; c1ccc(-c2nc3c(s2)-c2ccc(N(c4ccccc4)c4cccc5c4sc4ccccc45)c4cccc-3c24)cc1]

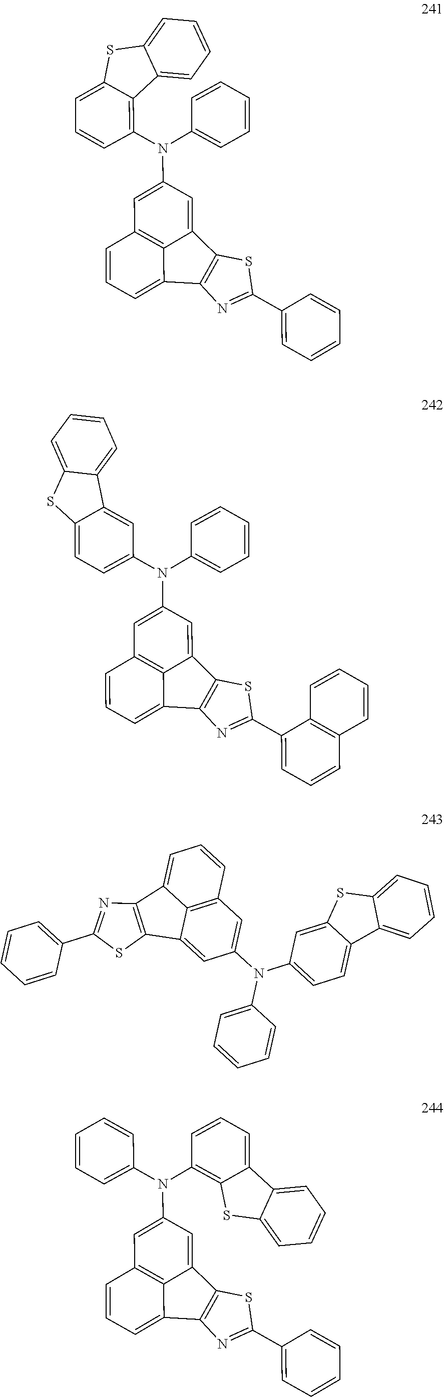 [SMILES: c1ccc(-c2nc3c(s2)-c2cc(N(c4ccccc4)c4cccc5sc6ccccc6c45)cc4cccc-3c24)cc1 ; c1ccc(N(c2cc3c4c(cccc4c2)-c2nc(-c4cccc5ccccc45)sc2-3)c2ccc3sc4ccccc4c3c2)cc1 ; c1ccc(-c2nc3c(s2)-c2cc(N(c4ccccc4)c4ccc5c(c4)sc4ccccc45)cc4cccc-3c24)cc1 ; c1ccc(-c2nc3c(s2)-c2cc(N(c4ccccc4)c4cccc5c4sc4ccccc45)cc4cccc-3c24)cc1]

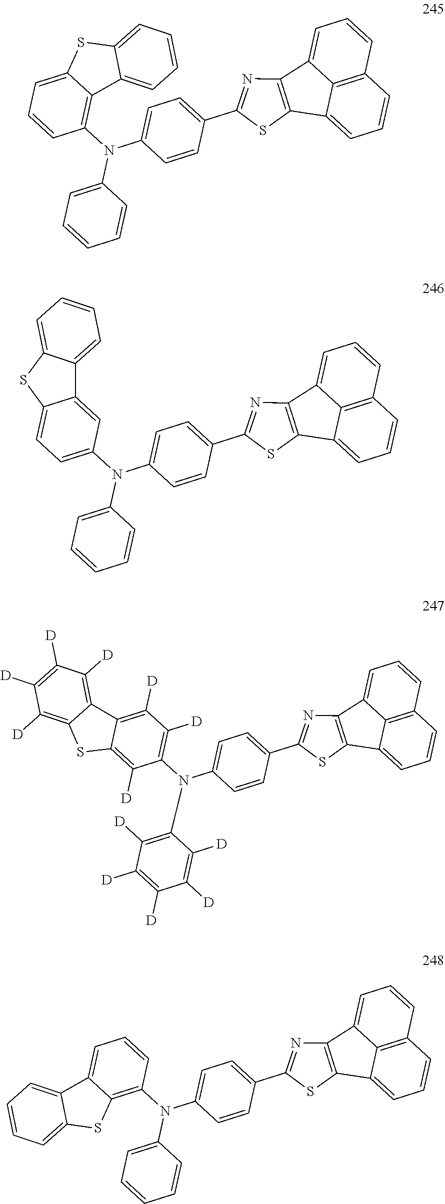 [SMILES: c1ccc(N(c2ccc(-c3nc4c(s3)-c3cccc5cccc-4c35)cc2)c2cccc3sc4ccccc4c23)cc1 ; c1ccc(N(c2ccc(-c3nc4c(s3)-c3cccc5cccc-4c35)cc2)c2ccc3sc4ccccc4c3c2)cc1 ; [2H]c1c([2H])c([2H])c(N(c2ccc(-c3nc4c(s3)-c3cccc5cccc-4c35)cc2)c2c([2H])c([2H])c3c(sc4c([2H])c([2H])c([2H])c([2H])c43)c2[2H])c([2H])c1[2H] ; c1ccc(N(c2ccc(-c3nc4c(s3)-c3cccc5cccc-4c35)cc2)c2cccc3c2sc2ccccc23)cc1]

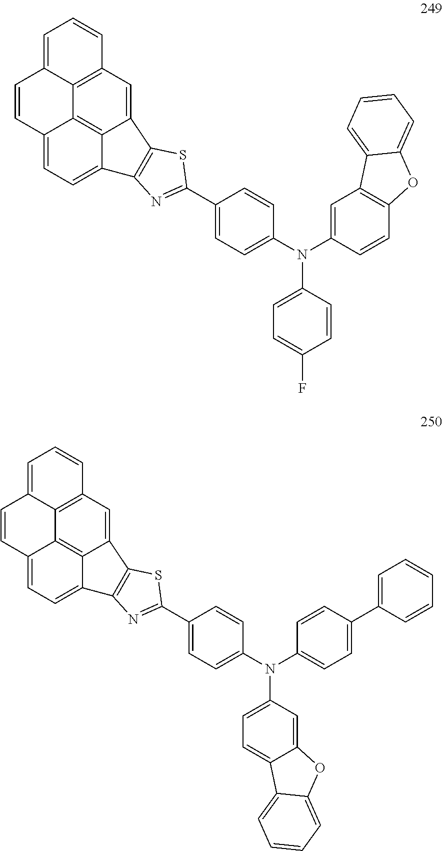 [SMILES: Fc1ccc(N(c2ccc(-c3nc4c(s3)-c3cc5cccc6ccc7ccc-4c3c7c65)cc2)c2ccc3oc4ccccc4c3c2)cc1 ; c1ccc(-c2ccc(N(c3ccc(-c4nc5c(s4)-c4cc6cccc7ccc8ccc-5c4c8c76)cc3)c3ccc4c(c3)oc3ccccc34)cc2)cc1]

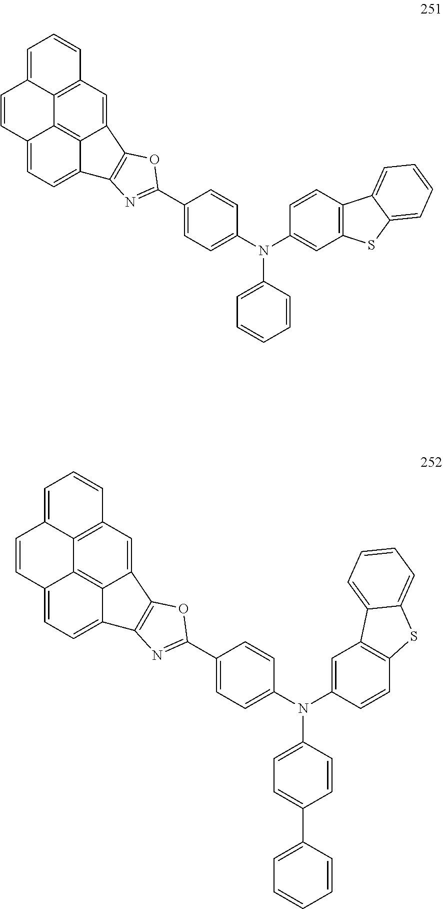 [SMILES: c1ccc(N(c2ccc(-c3nc4c(o3)-c3cc5cccc6ccc7ccc-4c3c7c65)cc2)c2ccc3c(c2)sc2ccccc23)cc1 ; c1ccc(-c2ccc(N(c3ccc(-c4nc5c(o4)-c4cc6cccc7ccc8ccc-5c4c8c76)cc3)c3ccc4sc5ccccc5c4c3)cc2)cc1]

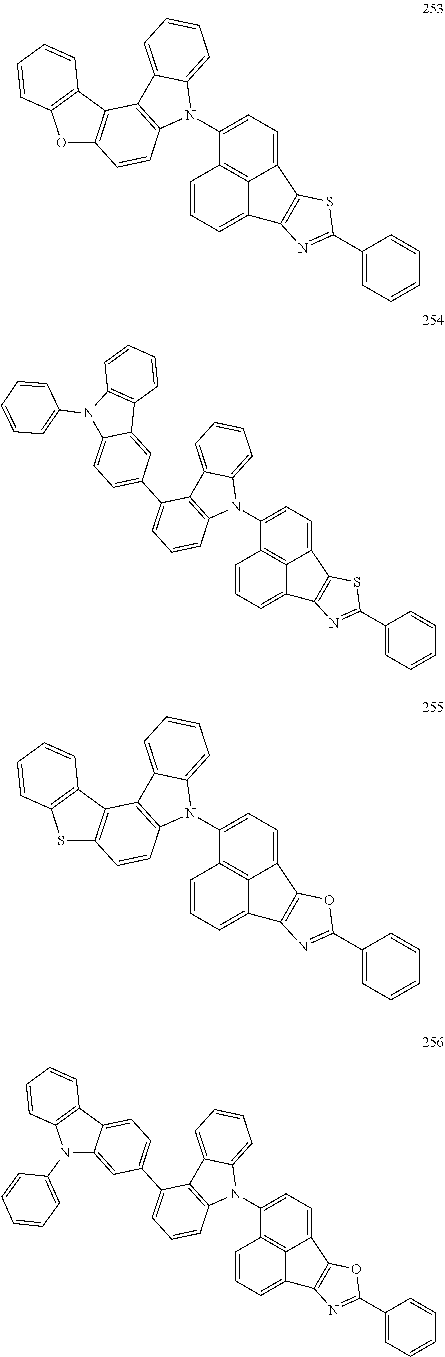 [SMILES: c1ccc(-c2nc3c(s2)-c2ccc(-n4c5ccccc5c5c6c(ccc54)oc4ccccc46)c4cccc-3c24)cc1 ; c1ccc(-c2nc3c(s2)-c2ccc(-n4c5ccccc5c5c(-c6ccc7c(c6)c6ccccc6n7-c6ccccc6)cccc54)c4cccc-3c24)cc1 ; c1ccc(-c2nc3c(o2)-c2ccc(-n4c5ccccc5c5c6c(ccc54)sc4ccccc46)c4cccc-3c24)cc1 ; c1ccc(-c2nc3c(o2)-c2ccc(-n4c5ccccc5c5c(-c6ccc7c8ccccc8n(-c8ccccc8)c7c6)cccc54)c4cccc-3c24)cc1]

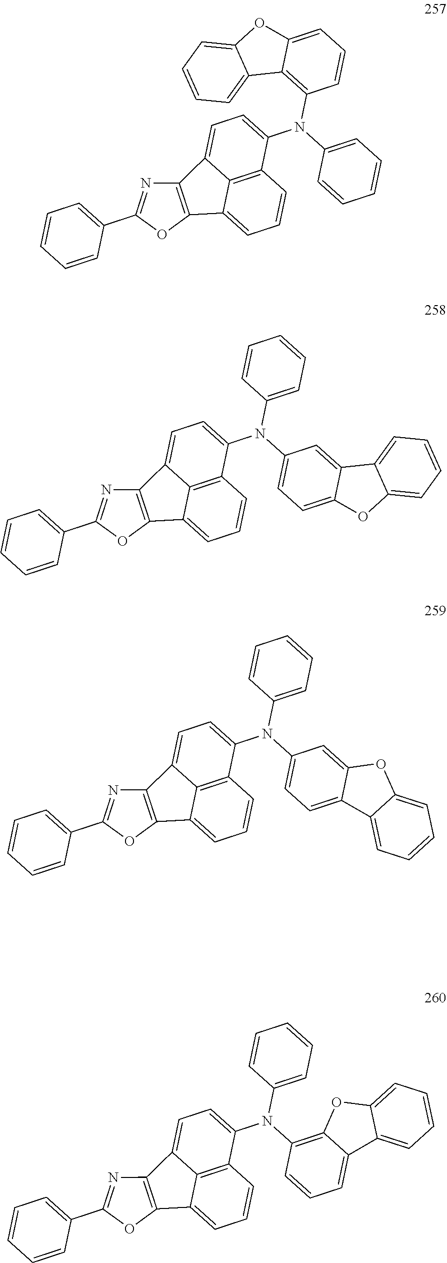 [SMILES: c1ccc(-c2nc3c(o2)-c2cccc4c(N(c5ccccc5)c5cccc6oc7ccccc7c56)ccc-3c24)cc1 ; c1ccc(-c2nc3c(o2)-c2cccc4c(N(c5ccccc5)c5ccc6oc7ccccc7c6c5)ccc-3c24)cc1 ; c1ccc(-c2nc3c(o2)-c2cccc4c(N(c5ccccc5)c5ccc6c(c5)oc5ccccc56)ccc-3c24)cc1 ; c1ccc(-c2nc3c(o2)-c2cccc4c(N(c5ccccc5)c5cccc6c5oc5ccccc56)ccc-3c24)cc1]

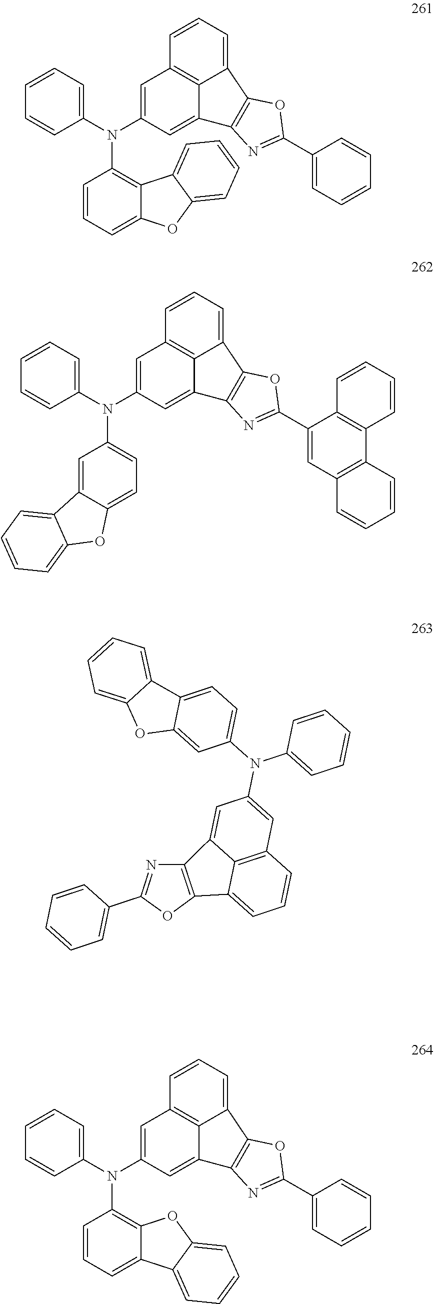 [SMILES: c1ccc(-c2nc3c(o2)-c2cccc4cc(N(c5ccccc5)c5cccc6oc7ccccc7c56)cc-3c24)cc1 ; c1ccc(N(c2cc3c4c(cccc4c2)-c2oc(-c4cc5ccccc5c5ccccc45)nc2-3)c2ccc3oc4ccccc4c3c2)cc1 ; c1ccc(-c2nc3c(o2)-c2cccc4cc(N(c5ccccc5)c5ccc6c(c5)oc5ccccc56)cc-3c24)cc1 ; c1ccc(-c2nc3c(o2)-c2cccc4cc(N(c5ccccc5)c5cccc6c5oc5ccccc56)cc-3c24)cc1]

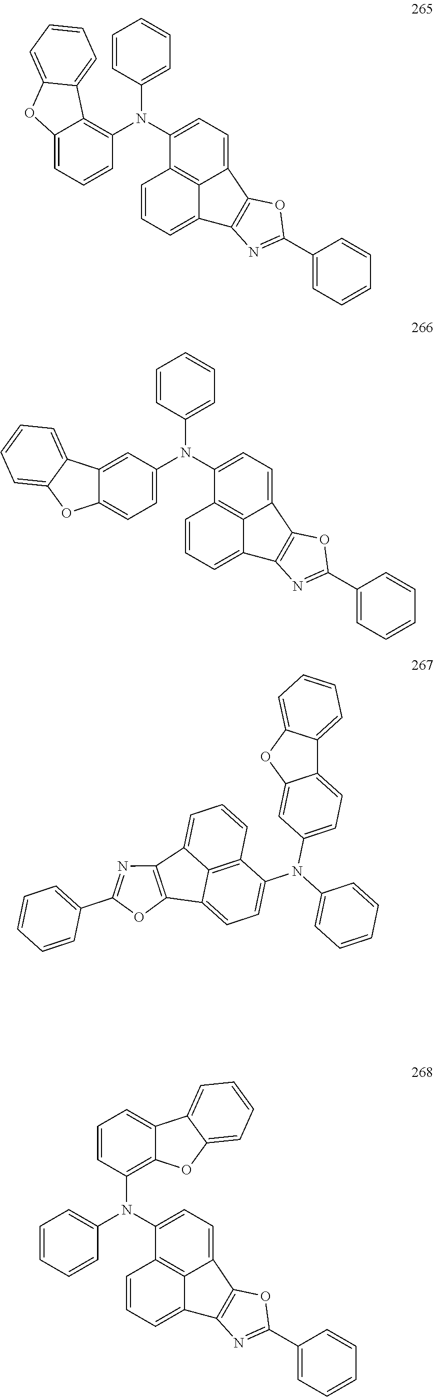 [SMILES: c1ccc(-c2nc3c(o2)-c2ccc(N(c4ccccc4)c4cccc5oc6ccccc6c45)c4cccc-3c24)cc1 ; c1ccc(-c2nc3c(o2)-c2ccc(N(c4ccccc4)c4ccc5oc6ccccc6c5c4)c4cccc-3c24)cc1 ; c1ccc(-c2nc3c(o2)-c2ccc(N(c4ccccc4)c4ccc5c(c4)oc4ccccc45)c4cccc-3c24)cc1 ; c1ccc(-c2nc3c(o2)-c2ccc(N(c4ccccc4)c4cccc5c4oc4ccccc45)c4cccc-3c24)cc1]

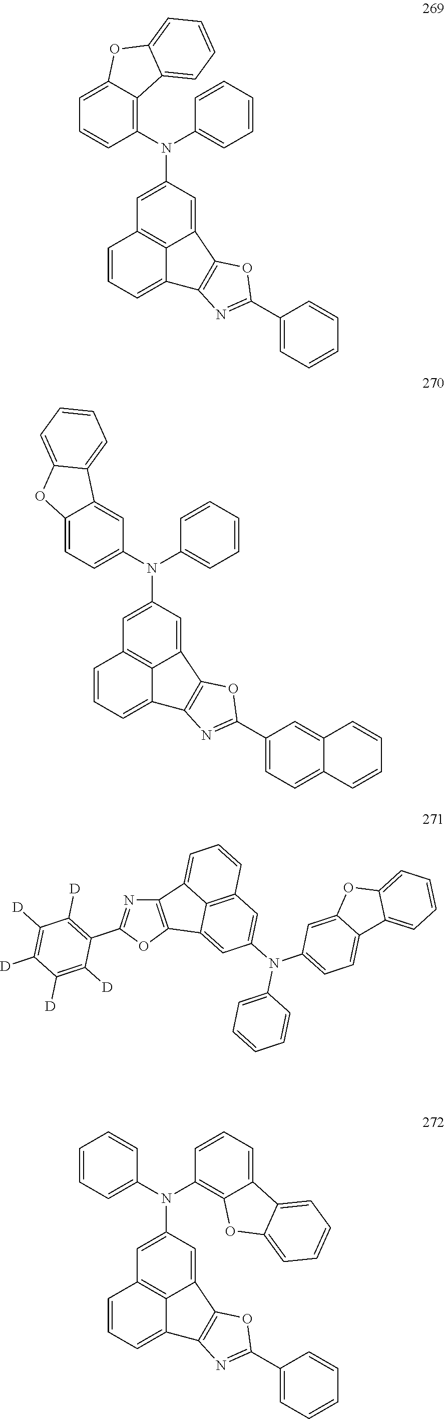 [SMILES: c1ccc(-c2nc3c(o2)-c2cc(N(c4ccccc4)c4cccc5oc6ccccc6c45)cc4cccc-3c24)cc1 ; c1ccc(N(c2cc3c4c(cccc4c2)-c2nc(-c4ccc5ccccc5c4)oc2-3)c2ccc3oc4ccccc4c3c2)cc1 ; [2H]c1c([2H])c([2H])c(-c2nc3c(o2)-c2cc(N(c4ccccc4)c4ccc5c(c4)oc4ccccc45)cc4cccc-3c24)c([2H])c1[2H] ; c1ccc(-c2nc3c(o2)-c2cc(N(c4ccccc4)c4cccc5c4oc4ccccc45)cc4cccc-3c24)cc1]

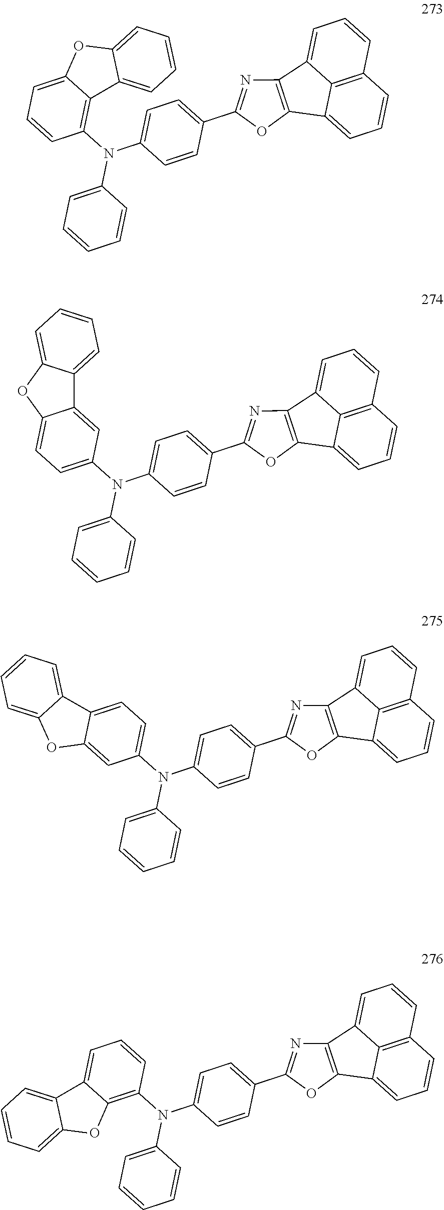 [SMILES: c1ccc(N(c2ccc(-c3nc4c(o3)-c3cccc5cccc-4c35)cc2)c2cccc3oc4ccccc4c23)cc1 ; c1ccc(N(c2ccc(-c3nc4c(o3)-c3cccc5cccc-4c35)cc2)c2ccc3oc4ccccc4c3c2)cc1 ; c1ccc(N(c2ccc(-c3nc4c(o3)-c3cccc5cccc-4c35)cc2)c2ccc3c(c2)oc2ccccc23)cc1 ; c1ccc(N(c2ccc(-c3nc4c(o3)-c3cccc5cccc-4c35)cc2)c2cccc3c2oc2ccccc23)cc1]

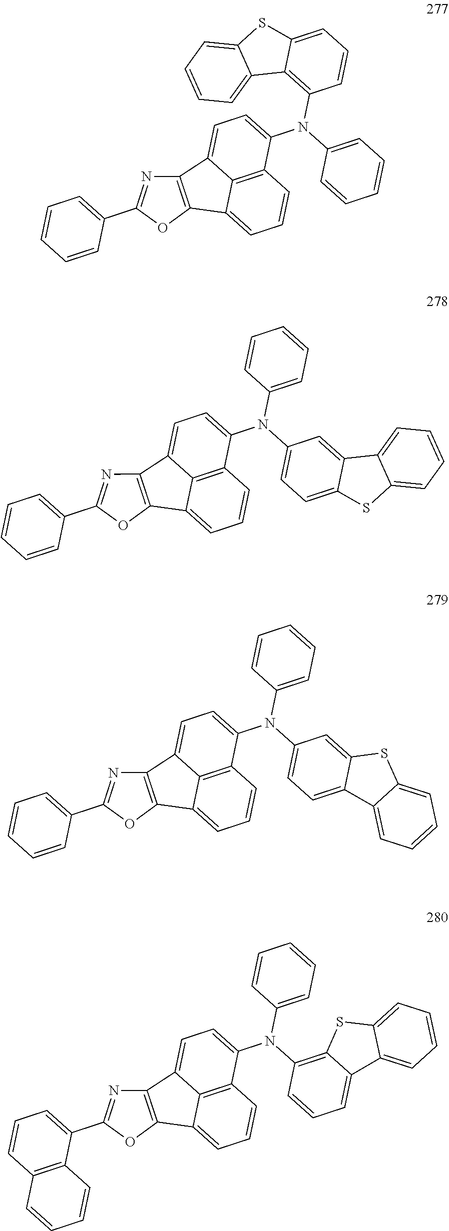 [SMILES: c1ccc(-c2nc3c(o2)-c2cccc4c(N(c5ccccc5)c5cccc6sc7ccccc7c56)ccc-3c24)cc1 ; c1ccc(-c2nc3c(o2)-c2cccc4c(N(c5ccccc5)c5ccc6sc7ccccc7c6c5)ccc-3c24)cc1 ; c1ccc(-c2nc3c(o2)-c2cccc4c(N(c5ccccc5)c5ccc6c(c5)sc5ccccc56)ccc-3c24)cc1 ; c1ccc(N(c2ccc3c4c(cccc24)-c2oc(-c4cccc5ccccc45)nc2-3)c2cccc3c2sc2ccccc23)cc1]

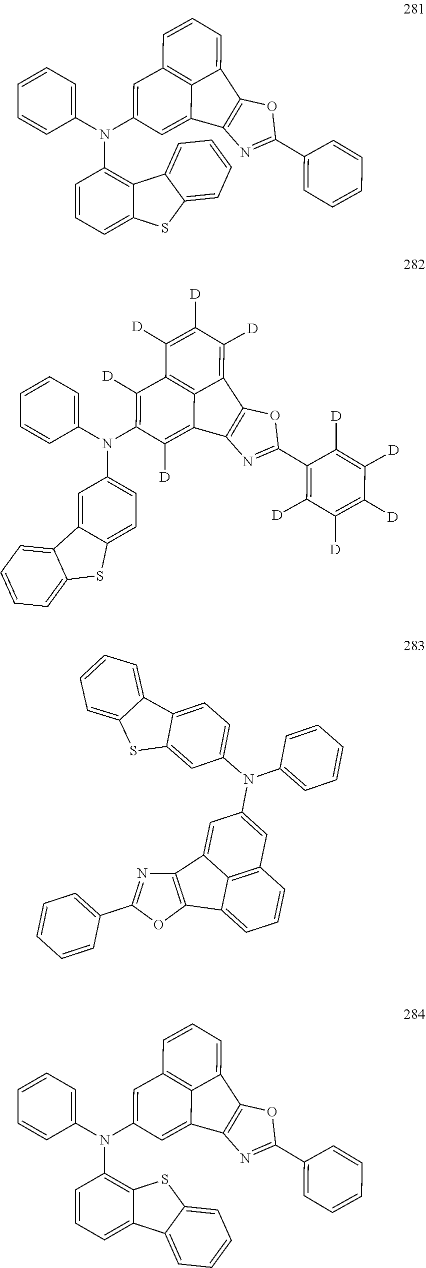 [SMILES: c1ccc(-c2nc3c(o2)-c2cccc4cc(N(c5ccccc5)c5cccc6sc7ccccc7c56)cc-3c24)cc1 ; [2H]c1c([2H])c([2H])c(-c2nc3c(o2)-c2c([2H])c([2H])c([2H])c4c([2H])c(N(c5ccccc5)c5ccc6sc7ccccc7c6c5)c([2H])c-3c24)c([2H])c1[2H] ; c1ccc(-c2nc3c(o2)-c2cccc4cc(N(c5ccccc5)c5ccc6c(c5)sc5ccccc56)cc-3c24)cc1 ; c1ccc(-c2nc3c(o2)-c2cccc4cc(N(c5ccccc5)c5cccc6c5sc5ccccc56)cc-3c24)cc1]

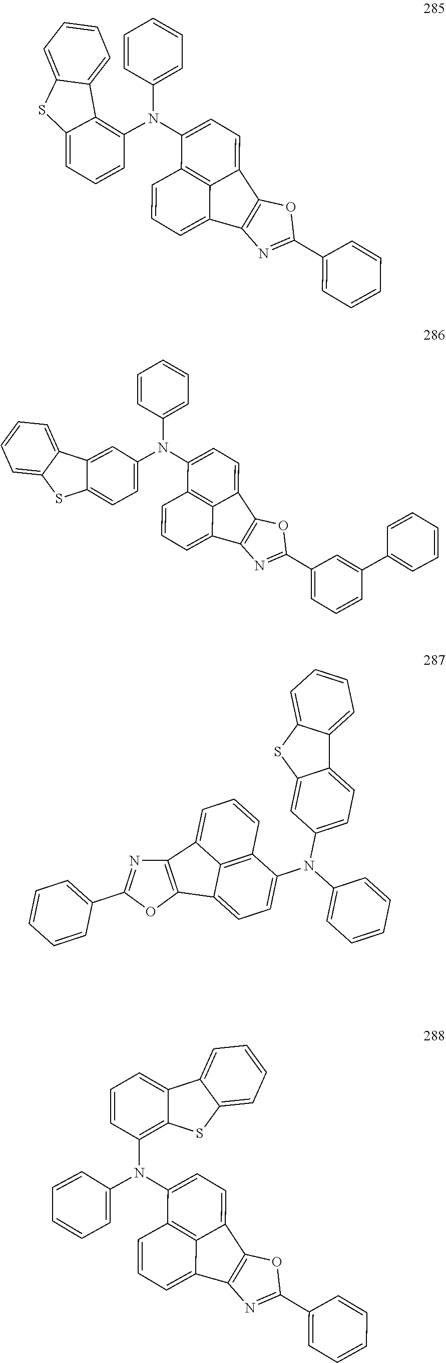 [SMILES: c1ccc(-c2nc3c(o2)-c2ccc(N(c4ccccc4)c4cccc5sc6ccccc6c45)c4cccc-3c24)cc1 ; c1ccc(-c2cccc(-c3nc4c(o3)-c3ccc(N(c5ccccc5)c5ccc6sc7ccccc7c6c5)c5cccc-4c35)c2)cc1 ; c1ccc(-c2nc3c(o2)-c2ccc(N(c4ccccc4)c4ccc5c(c4)sc4ccccc45)c4cccc-3c24)cc1 ; c1ccc(-c2nc3c(o2)-c2ccc(N(c4ccccc4)c4cccc5c4sc4ccccc45)c4cccc-3c24)cc1]

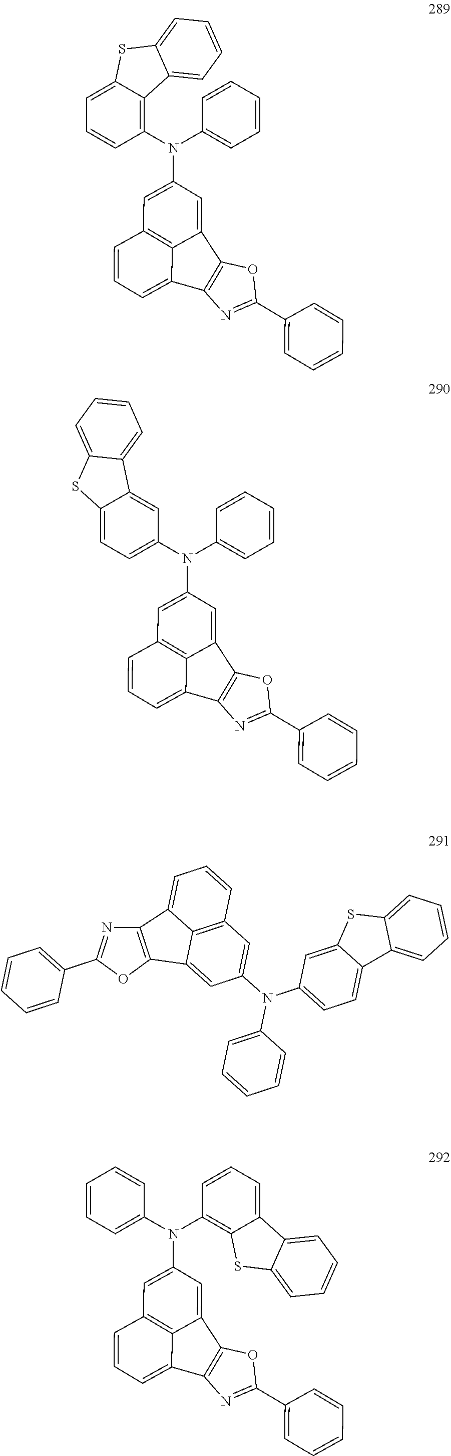 [SMILES: c1ccc(-c2nc3c(o2)-c2cc(N(c4ccccc4)c4cccc5sc6ccccc6c45)cc4cccc-3c24)cc1 ; c1ccc(-c2nc3c(o2)-c2cc(N(c4ccccc4)c4ccc5sc6ccccc6c5c4)cc4cccc-3c24)cc1 ; c1ccc(-c2nc3c(o2)-c2cc(N(c4ccccc4)c4ccc5c(c4)sc4ccccc45)cc4cccc-3c24)cc1 ; c1ccc(-c2nc3c(o2)-c2cc(N(c4ccccc4)c4cccc5c4sc4ccccc45)cc4cccc-3c24)cc1]

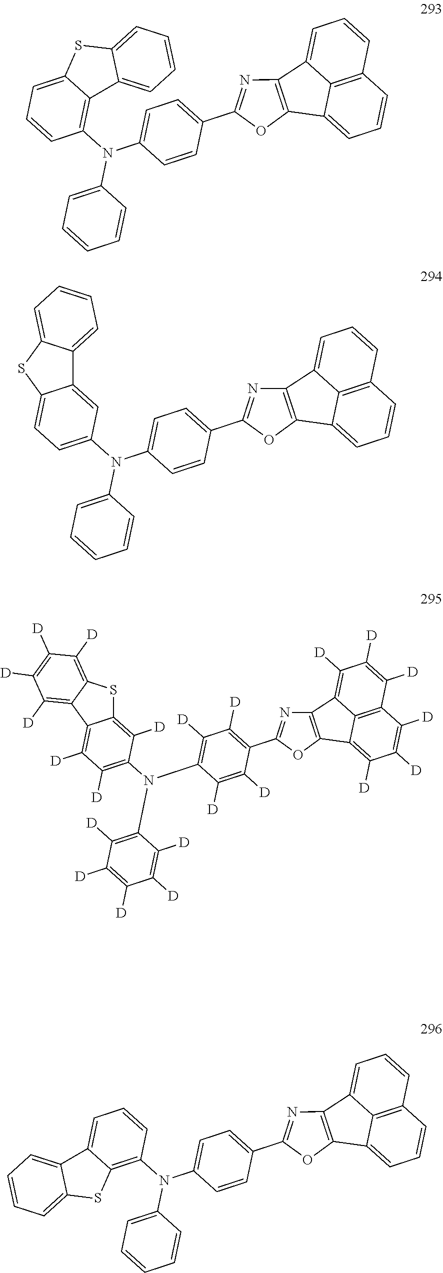 [SMILES: c1ccc(N(c2ccc(-c3nc4c(o3)-c3cccc5cccc-4c35)cc2)c2cccc3sc4ccccc4c23)cc1 ; c1ccc(N(c2ccc(-c3nc4c(o3)-c3cccc5cccc-4c35)cc2)c2ccc3sc4ccccc4c3c2)cc1 ; [2H]c1c([2H])c([2H])c(N(c2c([2H])c([2H])c(-c3nc4c(o3)-c3c([2H])c([2H])c([2H])c5c([2H])c([2H])c([2H])c-4c35)c([2H])c2[2H])c2c([2H])c([2H])c3c(sc4c([2H])c([2H])c([2H])c([2H])c43)c2[2H])c([2H])c1[2H] ; c1ccc(N(c2ccc(-c3nc4c(o3)-c3cccc5cccc-4c35)cc2)c2cccc3c2sc2ccccc23)cc1]

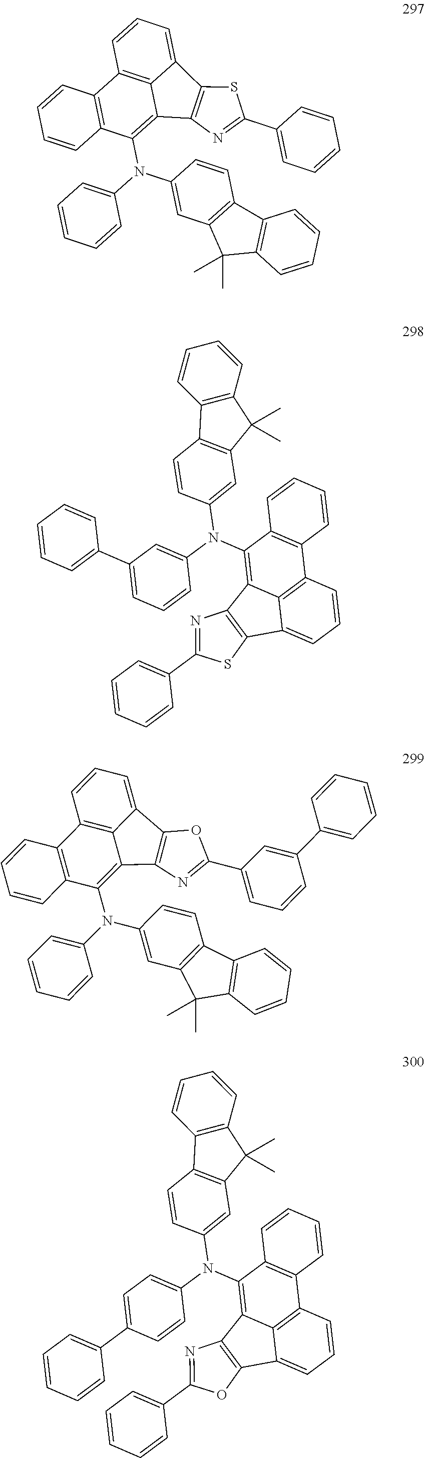

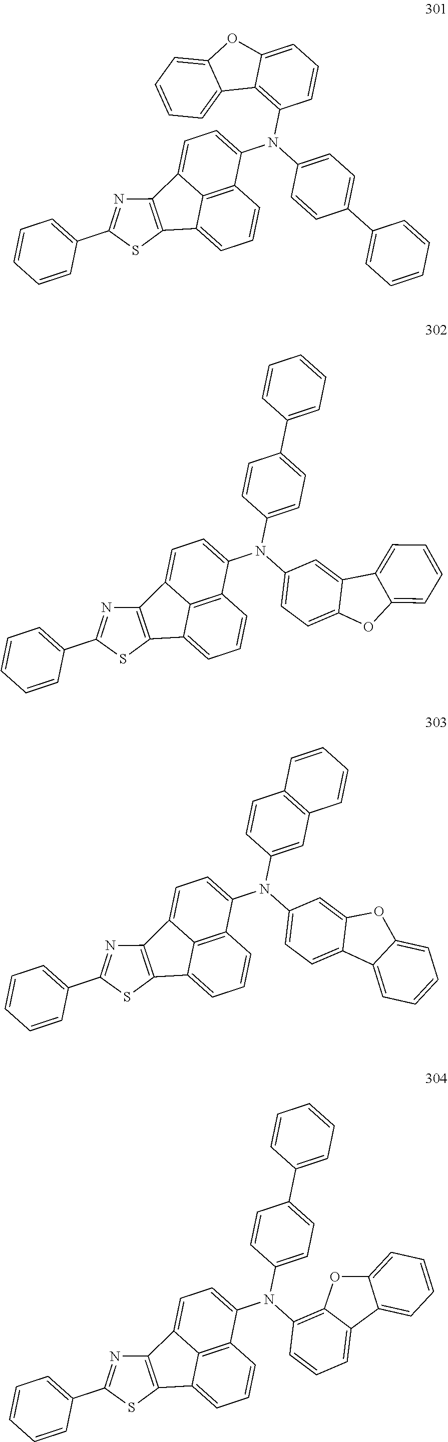 [SMILES: c1ccc(-c2ccc(N(c3ccc4c5c(cccc35)-c3sc(-c5ccccc5)nc3-4)c3cccc4oc5ccccc5c34)cc2)cc1 ; c1ccc(-c2ccc(N(c3ccc4oc5ccccc5c4c3)c3ccc4c5c(cccc35)-c3sc(-c5ccccc5)nc3-4)cc2)cc1 ; c1ccc(-c2nc3c(s2)-c2cccc4c(N(c5ccc6ccccc6c5)c5ccc6c(c5)oc5ccccc56)ccc-3c24)cc1 ; c1ccc(-c2ccc(N(c3ccc4c5c(cccc35)-c3sc(-c5ccccc5)nc3-4)c3cccc4c3oc3ccccc34)cc2)cc1]

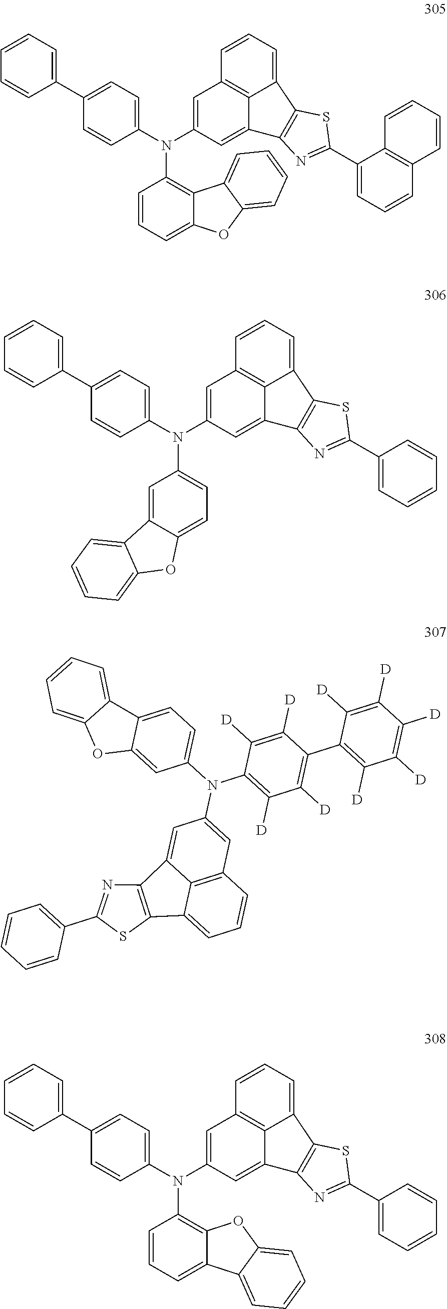 [SMILES: c1ccc(-c2ccc(N(c3cc4c5c(cccc5c3)-c3sc(-c5cccc6ccccc56)nc3-4)c3cccc4oc5ccccc5c34)cc2)cc1 ; c1ccc(-c2ccc(N(c3cc4c5c(cccc5c3)-c3sc(-c5ccccc5)nc3-4)c3ccc4oc5ccccc5c4c3)cc2)cc1 ; [2H]c1c([2H])c([2H])c(-c2c([2H])c([2H])c(N(c3cc4c5c(cccc5c3)-c3sc(-c5ccccc5)nc3-4)c3ccc4c(c3)oc3ccccc34)c([2H])c2[2H])c([2H])c1[2H] ; c1ccc(-c2ccc(N(c3cc4c5c(cccc5c3)-c3sc(-c5ccccc5)nc3-4)c3cccc4c3oc3ccccc34)cc2)cc1]

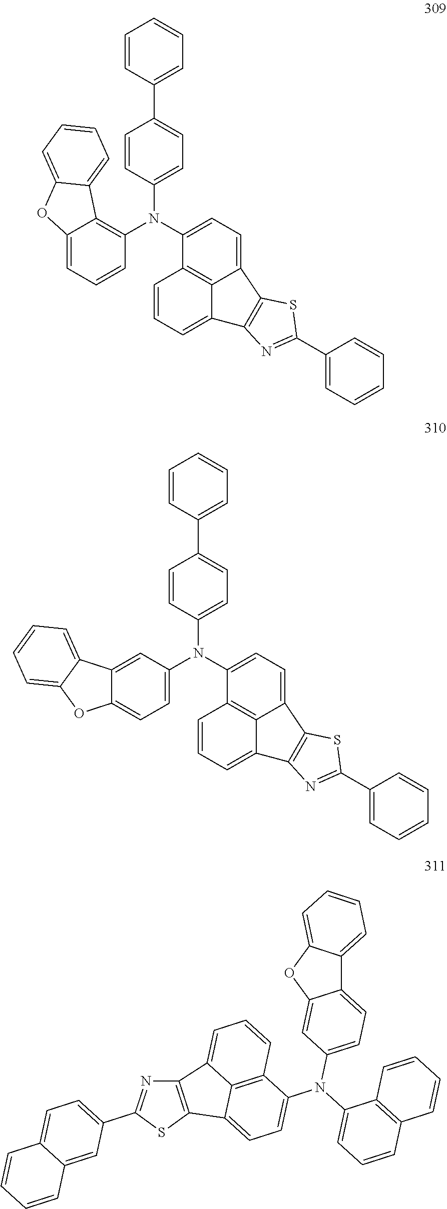 [SMILES: c1ccc(-c2ccc(N(c3ccc4c5c(cccc35)-c3nc(-c5ccccc5)sc3-4)c3cccc4oc5ccccc5c34)cc2)cc1 ; c1ccc(-c2ccc(N(c3ccc4oc5ccccc5c4c3)c3ccc4c5c(cccc35)-c3nc(-c5ccccc5)sc3-4)cc2)cc1 ; c1ccc2cc(-c3nc4c(s3)-c3ccc(N(c5ccc6c(c5)oc5ccccc56)c5cccc6ccccc56)c5cccc-4c35)ccc2c1]

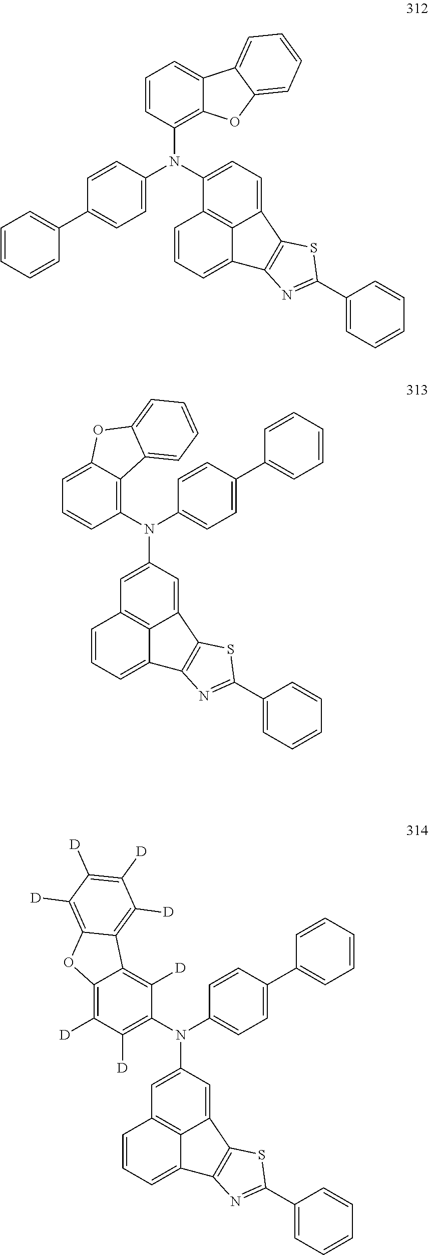 [SMILES: c1ccc(-c2ccc(N(c3ccc4c5c(cccc35)-c3nc(-c5ccccc5)sc3-4)c3cccc4c3oc3ccccc34)cc2)cc1 ; c1ccc(-c2ccc(N(c3cc4c5c(cccc5c3)-c3nc(-c5ccccc5)sc3-4)c3cccc4oc5ccccc5c34)cc2)cc1 ; [2H]c1c([2H])c([2H])c2c(oc3c([2H])c([2H])c(N(c4ccc(-c5ccccc5)cc4)c4cc5c6c(cccc6c4)-c4nc(-c6ccccc6)sc4-5)c([2H])c32)c1[2H]]

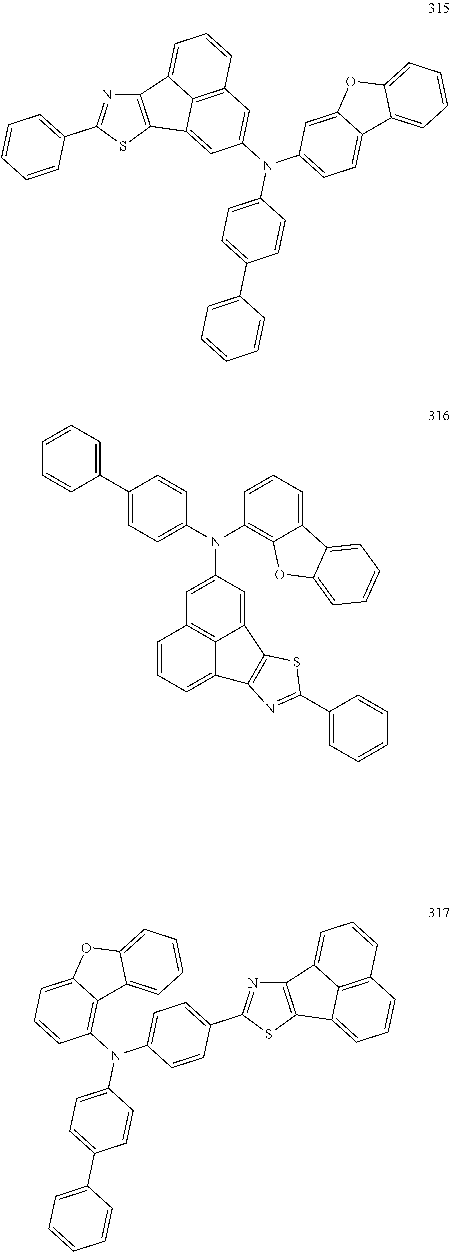 [SMILES: c1ccc(-c2ccc(N(c3cc4c5c(cccc5c3)-c3nc(-c5ccccc5)sc3-4)c3ccc4c(c3)oc3ccccc34)cc2)cc1 ; c1ccc(-c2ccc(N(c3cc4c5c(cccc5c3)-c3nc(-c5ccccc5)sc3-4)c3cccc4c3oc3ccccc34)cc2)cc1 ; c1ccc(-c2ccc(N(c3ccc(-c4nc5c(s4)-c4cccc6cccc-5c46)cc3)c3cccc4oc5ccccc5c34)cc2)cc1]

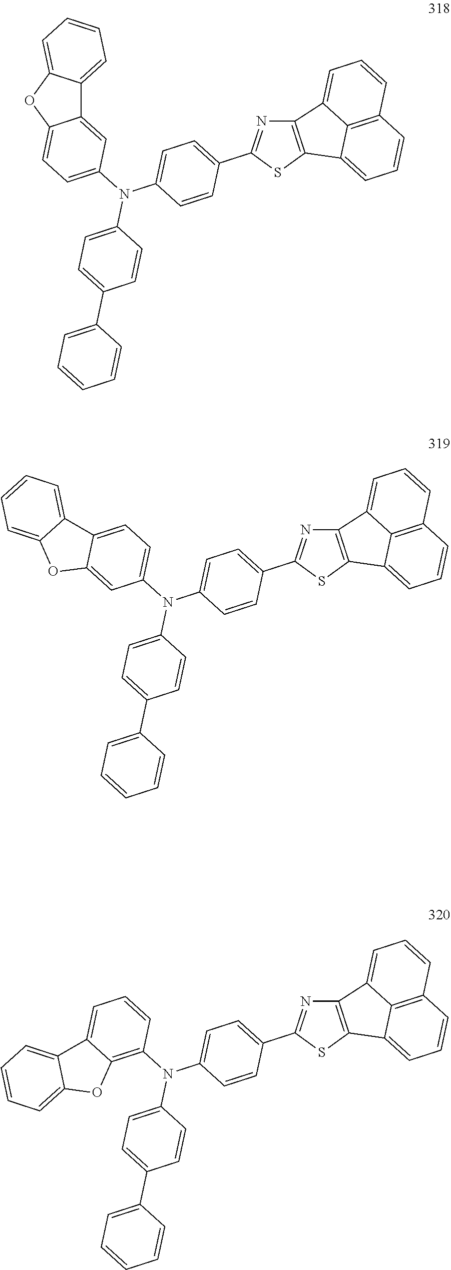 [SMILES: c1ccc(-c2ccc(N(c3ccc(-c4nc5c(s4)-c4cccc6cccc-5c46)cc3)c3ccc4oc5ccccc5c4c3)cc2)cc1 ; c1ccc(-c2ccc(N(c3ccc(-c4nc5c(s4)-c4cccc6cccc-5c46)cc3)c3ccc4c(c3)oc3ccccc34)cc2)cc1 ; c1ccc(-c2ccc(N(c3ccc(-c4nc5c(s4)-c4cccc6cccc-5c46)cc3)c3cccc4c3oc3ccccc34)cc2)cc1]

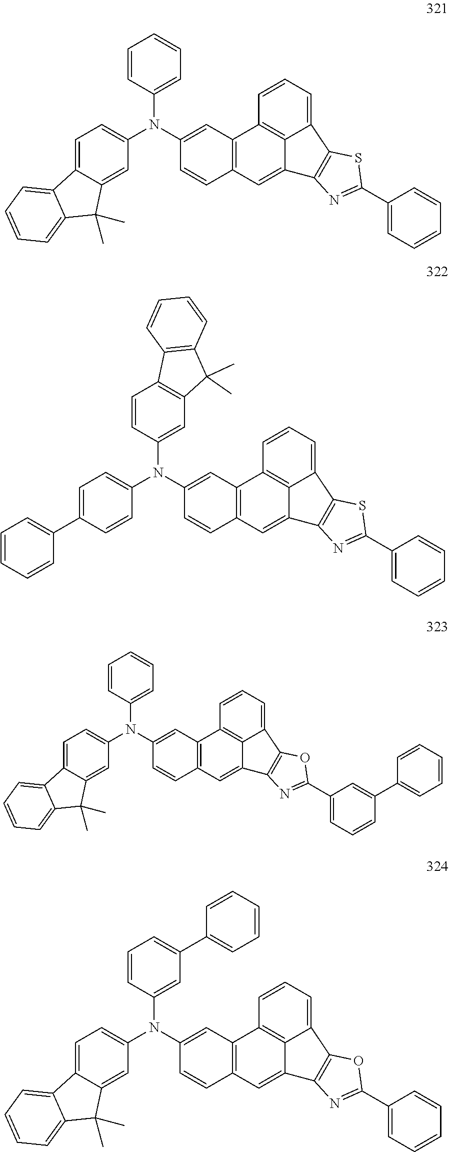 [SMILES: CC1(C)c2ccccc2-c2ccc(N(c3ccccc3)c3ccc4cc5c6c(cccc6c4c3)-c3sc(-c4ccccc4)nc3-5)cc21 ; CC1(C)c2ccccc2-c2ccc(N(c3ccc(-c4ccccc4)cc3)c3ccc4cc5c6c(cccc6c4c3)-c3sc(-c4ccccc4)nc3-5)cc21 ; CC1(C)c2ccccc2-c2ccc(N(c3ccccc3)c3ccc4cc5c6c(cccc6c4c3)-c3oc(-c4cccc(-c6ccccc6)c4)nc3-5)cc21 ; CC1(C)c2ccccc2-c2ccc(N(c3cccc(-c4ccccc4)c3)c3ccc4cc5c6c(cccc6c4c3)-c3oc(-c4ccccc4)nc3-5)cc21]

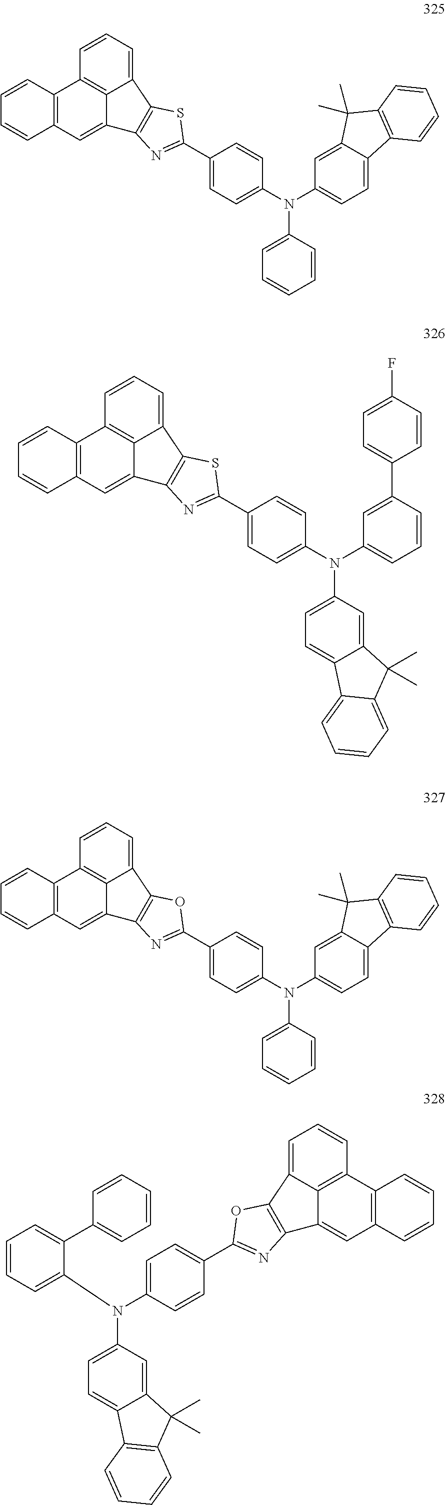 [SMILES: CC1(C)c2ccccc2-c2ccc(N(c3ccccc3)c3ccc(-c4nc5c(s4)-c4cccc6c4c-5cc4ccccc46)cc3)cc21 ; CC1(C)c2ccccc2-c2ccc(N(c3ccc(-c4nc5c(s4)-c4cccc6c4c-5cc4ccccc46)cc3)c3cccc(-c4ccc(F)cc4)c3)cc21 ; CC1(C)c2ccccc2-c2ccc(N(c3ccccc3)c3ccc(-c4nc5c(o4)-c4cccc6c4c-5cc4ccccc46)cc3)cc21 ; CC1(C)c2ccccc2-c2ccc(N(c3ccc(-c4nc5c(o4)-c4cccc6c4c-5cc4ccccc46)cc3)c3ccccc3-c3ccccc3)cc21]

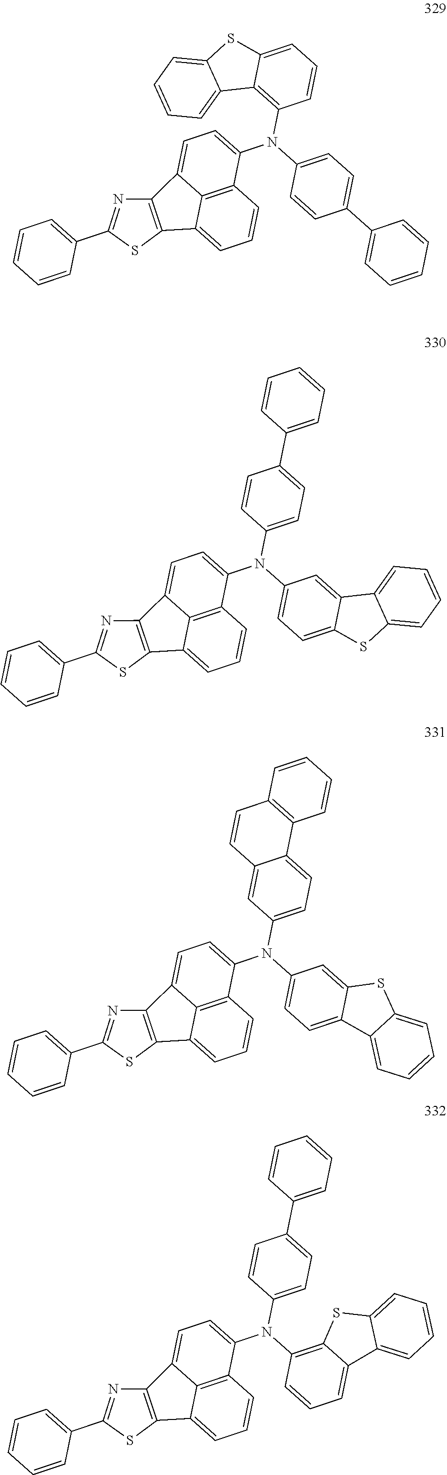 [SMILES: c1ccc(-c2ccc(N(c3ccc4c5c(cccc35)-c3sc(-c5ccccc5)nc3-4)c3cccc4sc5ccccc5c34)cc2)cc1 ; c1ccc(-c2ccc(N(c3ccc4sc5ccccc5c4c3)c3ccc4c5c(cccc35)-c3sc(-c5ccccc5)nc3-4)cc2)cc1 ; c1ccc(-c2nc3c(s2)-c2cccc4c(N(c5ccc6c(ccc7ccccc76)c5)c5ccc6c(c5)sc5ccccc56)ccc-3c24)cc1 ; c1ccc(-c2ccc(N(c3ccc4c5c(cccc35)-c3sc(-c5ccccc5)nc3-4)c3cccc4c3sc3ccccc34)cc2)cc1]

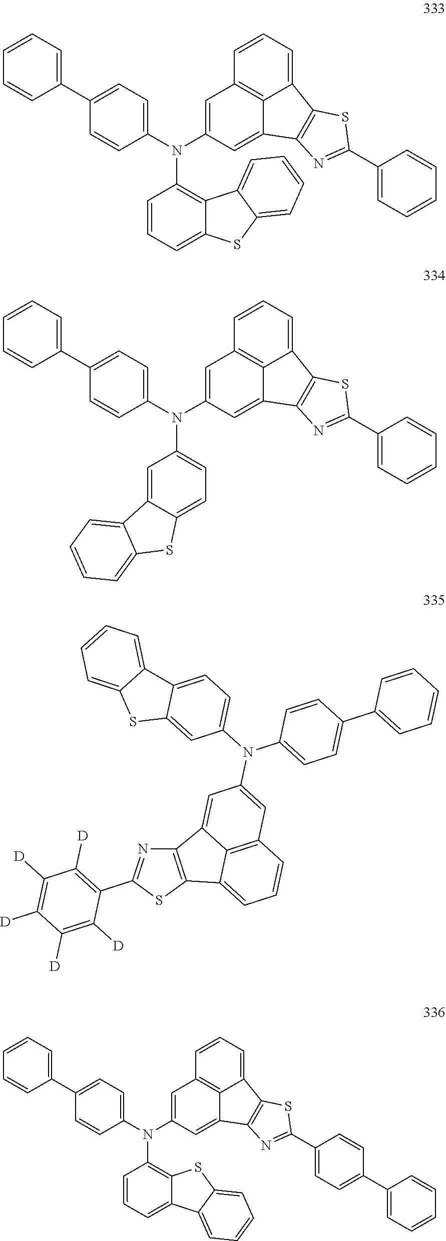

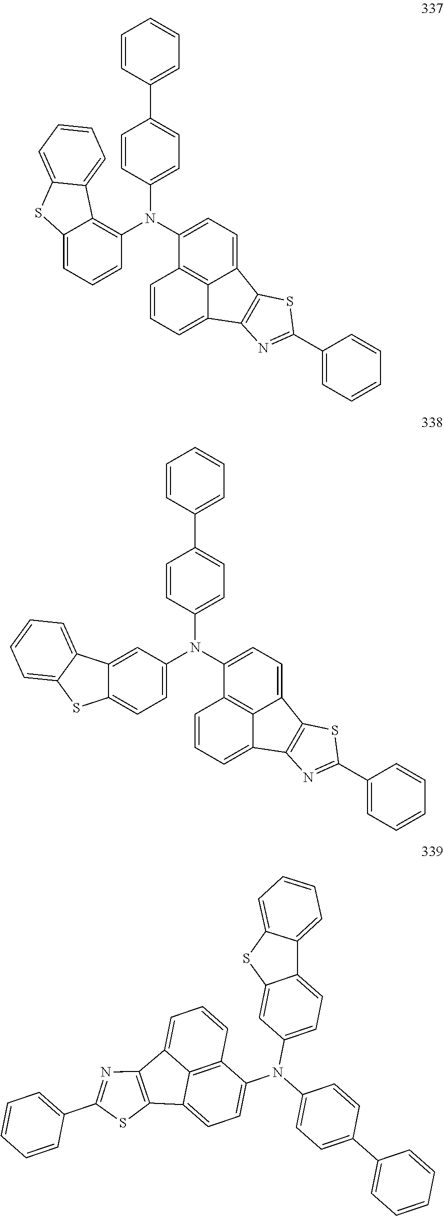 [SMILES: c1ccc(-c2ccc(N(c3ccc4c5c(cccc35)-c3nc(-c5ccccc5)sc3-4)c3cccc4sc5ccccc5c34)cc2)cc1 ; c1ccc(-c2ccc(N(c3ccc4sc5ccccc5c4c3)c3ccc4c5c(cccc35)-c3nc(-c5ccccc5)sc3-4)cc2)cc1 ; c1ccc(-c2ccc(N(c3ccc4c(c3)sc3ccccc34)c3ccc4c5c(cccc35)-c3nc(-c5ccccc5)sc3-4)cc2)cc1]

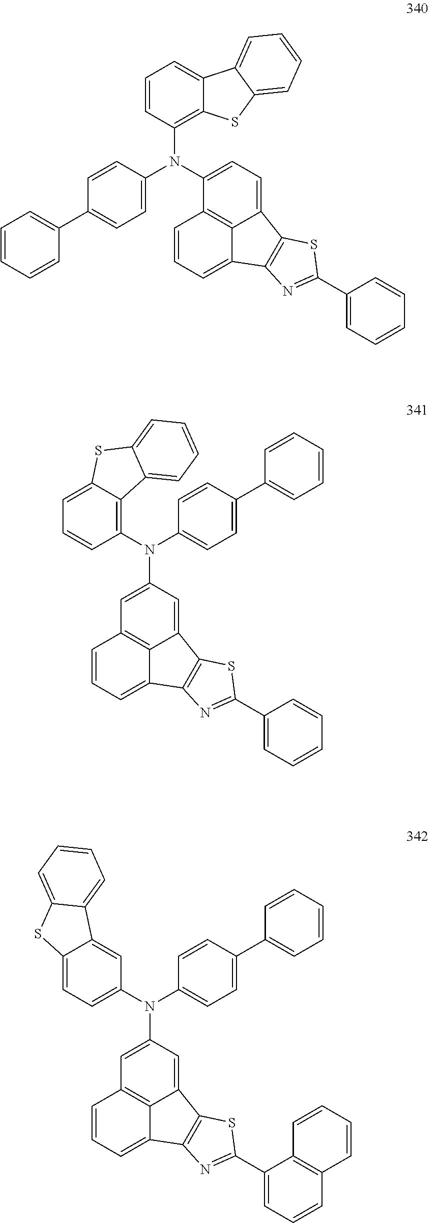 [SMILES: c1ccc(-c2ccc(N(c3ccc4c5c(cccc35)-c3nc(-c5ccccc5)sc3-4)c3cccc4c3sc3ccccc34)cc2)cc1 ; c1ccc(-c2ccc(N(c3cc4c5c(cccc5c3)-c3nc(-c5ccccc5)sc3-4)c3cccc4sc5ccccc5c34)cc2)cc1 ; c1ccc(-c2ccc(N(c3cc4c5c(cccc5c3)-c3nc(-c5cccc6ccccc56)sc3-4)c3ccc4sc5ccccc5c4c3)cc2)cc1]

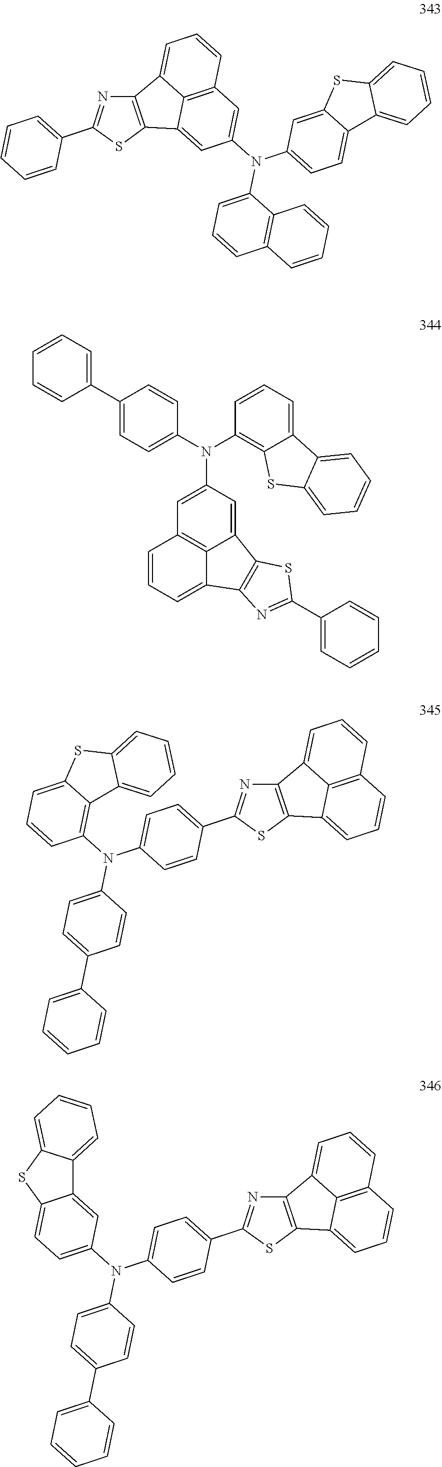

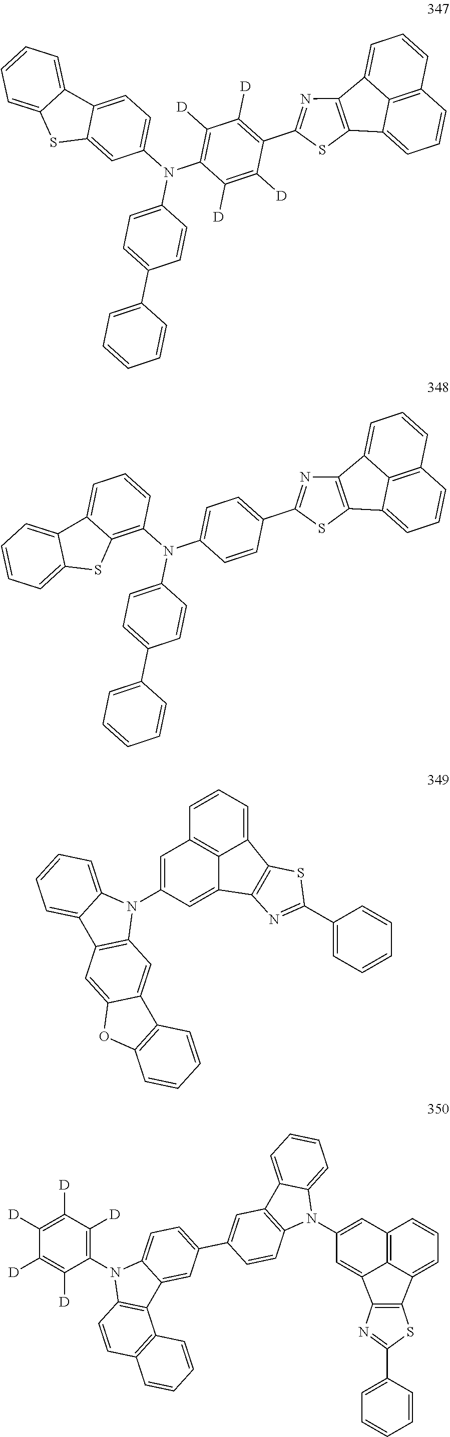 [SMILES: [2H]c1c([2H])c(N(c2ccc(-c3ccccc3)cc2)c2ccc3c(c2)sc2ccccc23)c([2H])c([2H])c1-c1nc2c(s1)-c1cccc3cccc-2c13 ; c1ccc(-c2ccc(N(c3ccc(-c4nc5c(s4)-c4cccc6cccc-5c46)cc3)c3cccc4c3sc3ccccc34)cc2)cc1 ; c1ccc(-c2nc3c(s2)-c2cccc4cc(-n5c6ccccc6c6cc7oc8ccccc8c7cc65)cc-3c24)cc1 ; [2H]c1c([2H])c([2H])c(-n2c3ccc(-c4ccc5c(c4)c4ccccc4n5-c4cc5c6c(cccc6c4)-c4sc(-c6ccccc6)nc4-5)cc3c3c4ccccc4ccc32)c([2H])c1[2H]]

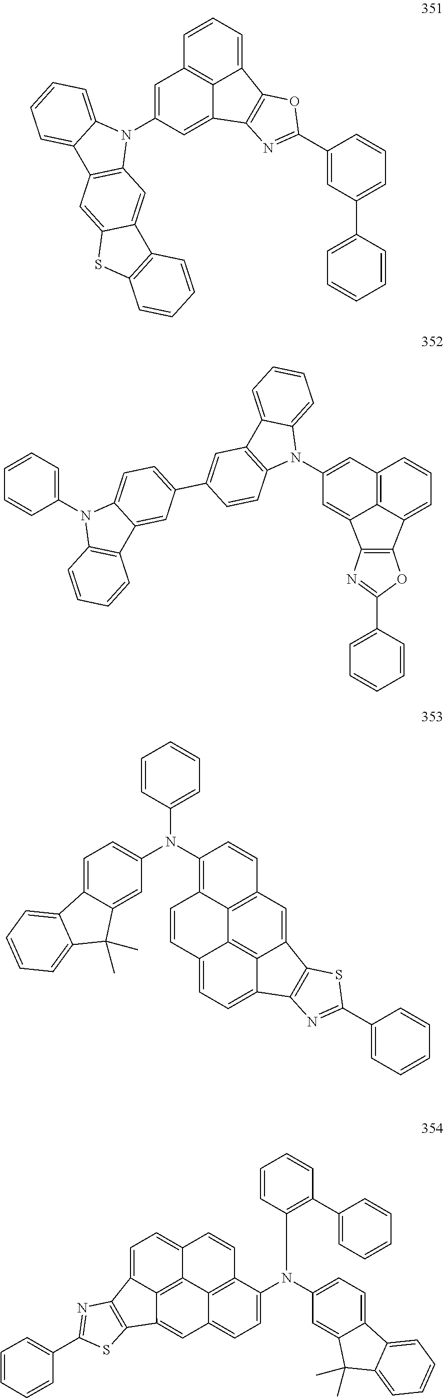 [SMILES: c1ccc(-c2cccc(-c3nc4c(o3)-c3cccc5cc(-n6c7ccccc7c7cc8sc9ccccc9c8cc76)cc-4c35)c2)cc1 ; c1ccc(-c2nc3c(o2)-c2cccc4cc(-n5c6ccccc6c6cc(-c7ccc8c(c7)c7ccccc7n8-c7ccccc7)ccc65)cc-3c24)cc1 ; CC1(C)c2ccccc2-c2ccc(N(c3ccccc3)c3ccc4cc5c6c(ccc7ccc3c4c76)-c3nc(-c4ccccc4)sc3-5)cc21 ; CC1(C)c2ccccc2-c2ccc(N(c3ccccc3-c3ccccc3)c3ccc4cc5c6c(ccc7ccc3c4c76)-c3nc(-c4ccccc4)sc3-5)cc21]

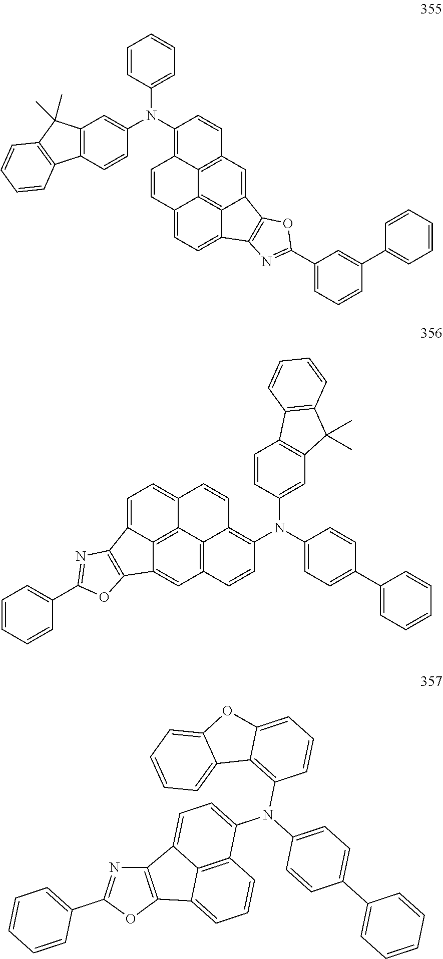 [SMILES: CC1(C)c2ccccc2-c2ccc(N(c3ccccc3)c3ccc4cc5c6c(ccc7ccc3c4c76)-c3nc(-c4cccc(-c6ccccc6)c4)oc3-5)cc21 ; CC1(C)c2ccccc2-c2ccc(N(c3ccc(-c4ccccc4)cc3)c3ccc4cc5c6c(ccc7ccc3c4c76)-c3nc(-c4ccccc4)oc3-5)cc21 ; c1ccc(-c2ccc(N(c3ccc4c5c(cccc35)-c3oc(-c5ccccc5)nc3-4)c3cccc4oc5ccccc5c34)cc2)cc1]

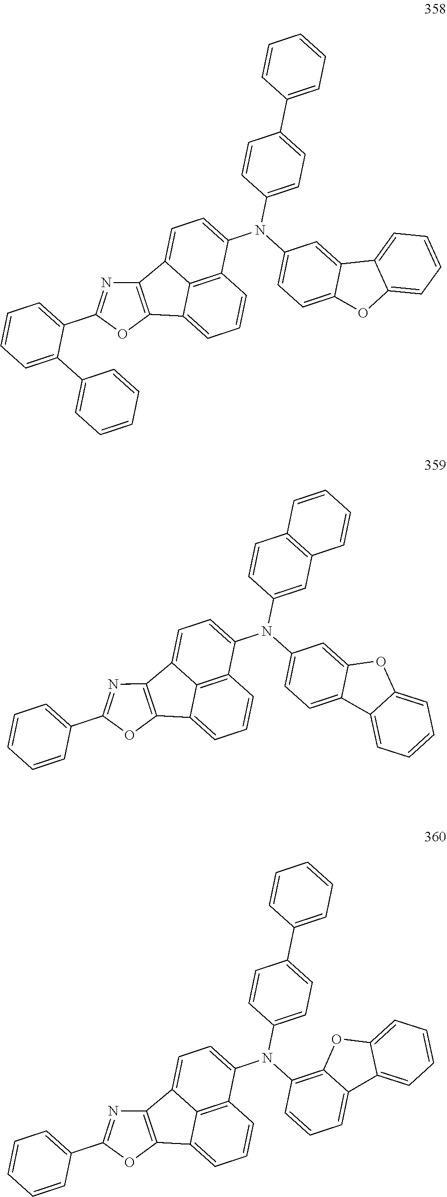 [SMILES: c1ccc(-c2ccc(N(c3ccc4oc5ccccc5c4c3)c3ccc4c5c(cccc35)-c3oc(-c5ccccc5-c5ccccc5)nc3-4)cc2)cc1 ; c1ccc(-c2nc3c(o2)-c2cccc4c(N(c5ccc6ccccc6c5)c5ccc6c(c5)oc5ccccc56)ccc-3c24)cc1 ; c1ccc(-c2ccc(N(c3ccc4c5c(cccc35)-c3oc(-c5ccccc5)nc3-4)c3cccc4c3oc3ccccc34)cc2)cc1]

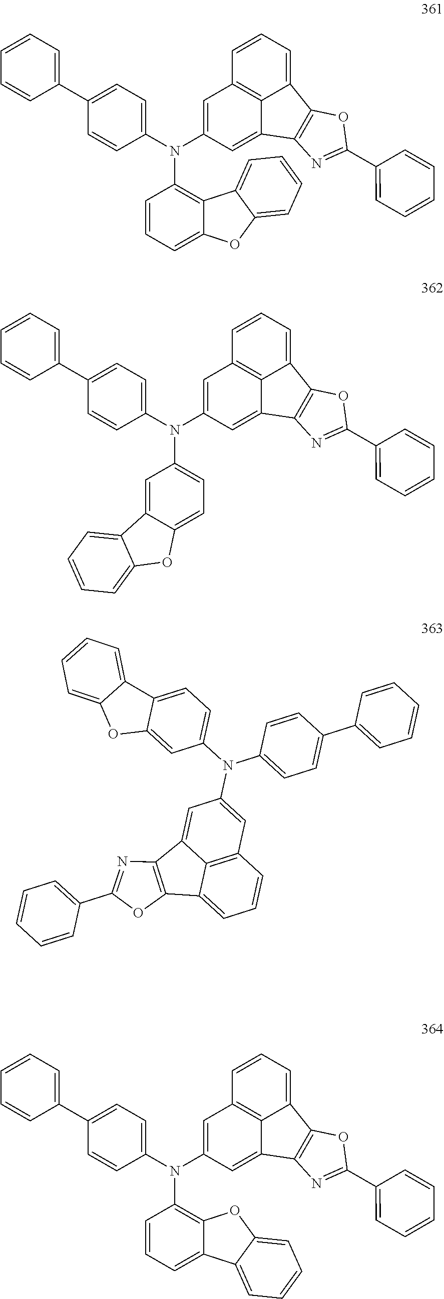 [SMILES: c1ccc(-c2ccc(N(c3cc4c5c(cccc5c3)-c3oc(-c5ccccc5)nc3-4)c3cccc4oc5ccccc5c34)cc2)cc1 ; c1ccc(-c2ccc(N(c3cc4c5c(cccc5c3)-c3oc(-c5ccccc5)nc3-4)c3ccc4oc5ccccc5c4c3)cc2)cc1 ; c1ccc(-c2ccc(N(c3cc4c5c(cccc5c3)-c3oc(-c5ccccc5)nc3-4)c3ccc4c(c3)oc3ccccc34)cc2)cc1 ; c1ccc(-c2ccc(N(c3cc4c5c(cccc5c3)-c3oc(-c5ccccc5)nc3-4)c3cccc4c3oc3ccccc34)cc2)cc1]

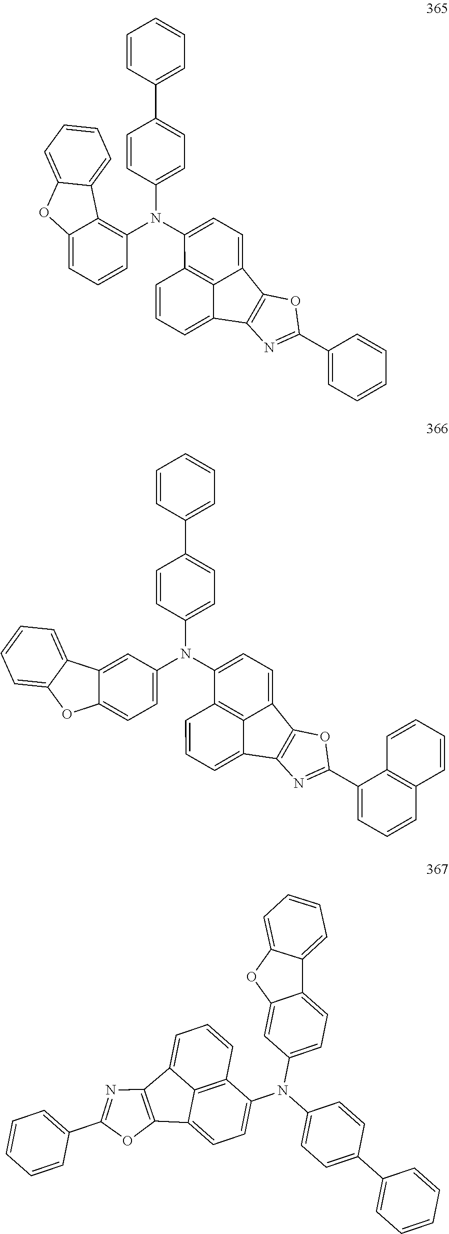 [SMILES: c1ccc(-c2ccc(N(c3ccc4c5c(cccc35)-c3nc(-c5ccccc5)oc3-4)c3cccc4oc5ccccc5c34)cc2)cc1 ; c1ccc(-c2ccc(N(c3ccc4oc5ccccc5c4c3)c3ccc4c5c(cccc35)-c3nc(-c5cccc6ccccc56)oc3-4)cc2)cc1 ; c1ccc(-c2ccc(N(c3ccc4c(c3)oc3ccccc34)c3ccc4c5c(cccc35)-c3nc(-c5ccccc5)oc3-4)cc2)cc1]

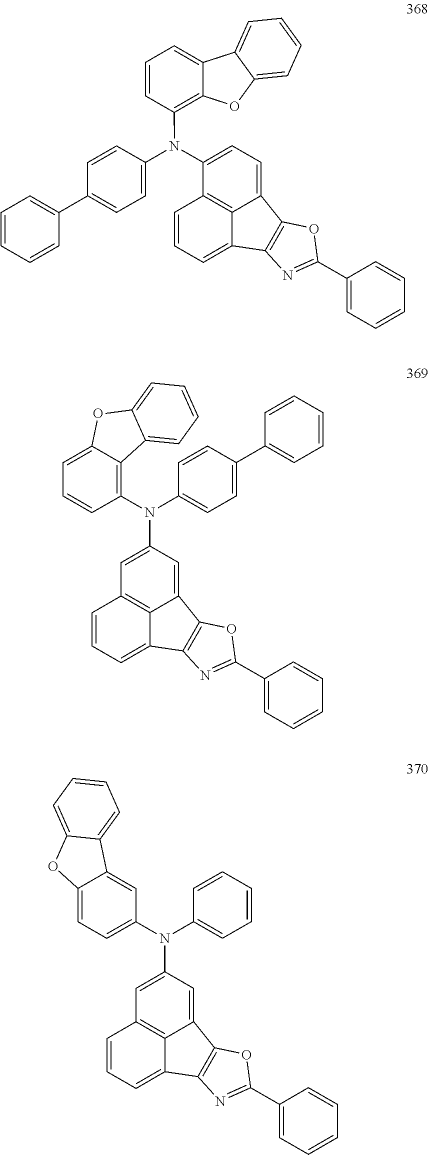 [SMILES: c1ccc(-c2ccc(N(c3ccc4c5c(cccc35)-c3nc(-c5ccccc5)oc3-4)c3cccc4c3oc3ccccc34)cc2)cc1 ; c1ccc(-c2ccc(N(c3cc4c5c(cccc5c3)-c3nc(-c5ccccc5)oc3-4)c3cccc4oc5ccccc5c34)cc2)cc1 ; c1ccc(-c2nc3c(o2)-c2cc(N(c4ccccc4)c4ccc5oc6ccccc6c5c4)cc4cccc-3c24)cc1]

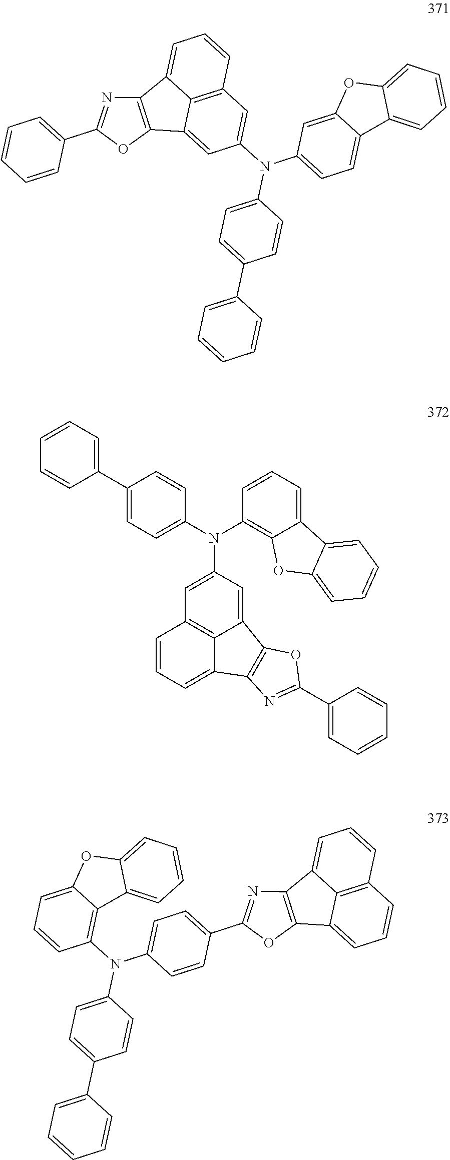 [SMILES: c1ccc(-c2ccc(N(c3cc4c5c(cccc5c3)-c3nc(-c5ccccc5)oc3-4)c3ccc4c(c3)oc3ccccc34)cc2)cc1 ; c1ccc(-c2ccc(N(c3cc4c5c(cccc5c3)-c3nc(-c5ccccc5)oc3-4)c3cccc4c3oc3ccccc34)cc2)cc1 ; c1ccc(-c2ccc(N(c3ccc(-c4nc5c(o4)-c4cccc6cccc-5c46)cc3)c3cccc4oc5ccccc5c34)cc2)cc1]

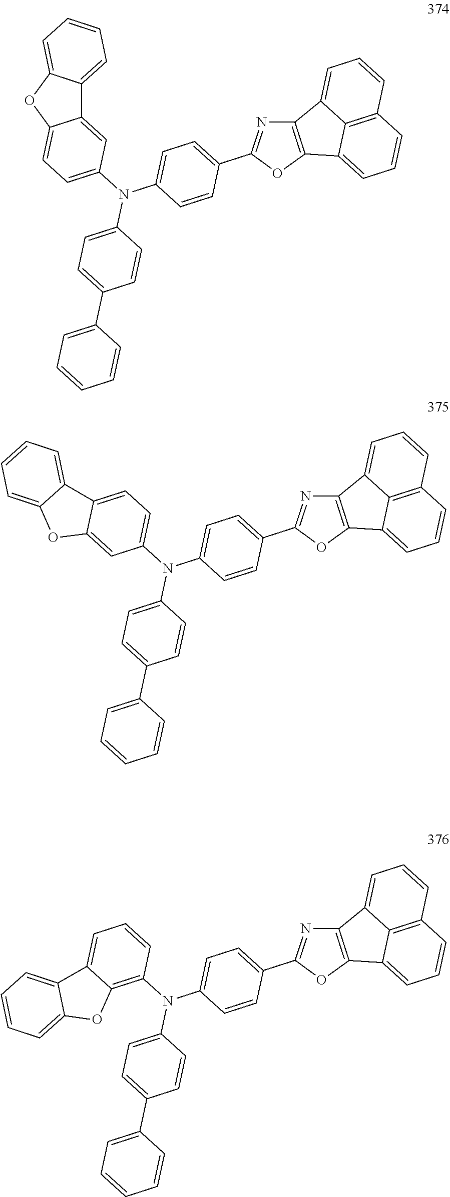 [SMILES: c1ccc(-c2ccc(N(c3ccc(-c4nc5c(o4)-c4cccc6cccc-5c46)cc3)c3ccc4oc5ccccc5c4c3)cc2)cc1 ; c1ccc(-c2ccc(N(c3ccc(-c4nc5c(o4)-c4cccc6cccc-5c46)cc3)c3ccc4c(c3)oc3ccccc34)cc2)cc1 ; c1ccc(-c2ccc(N(c3ccc(-c4nc5c(o4)-c4cccc6cccc-5c46)cc3)c3cccc4c3oc3ccccc34)cc2)cc1]

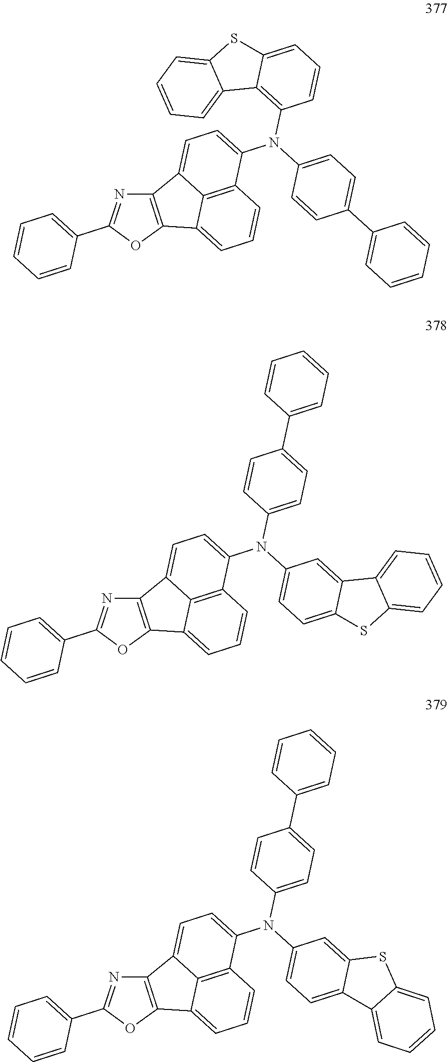 [SMILES: c1ccc(-c2ccc(N(c3ccc4c5c(cccc35)-c3oc(-c5ccccc5)nc3-4)c3cccc4sc5ccccc5c34)cc2)cc1 ; c1ccc(-c2ccc(N(c3ccc4sc5ccccc5c4c3)c3ccc4c5c(cccc35)-c3oc(-c5ccccc5)nc3-4)cc2)cc1 ; c1ccc(-c2ccc(N(c3ccc4c(c3)sc3ccccc34)c3ccc4c5c(cccc35)-c3oc(-c5ccccc5)nc3-4)cc2)cc1]

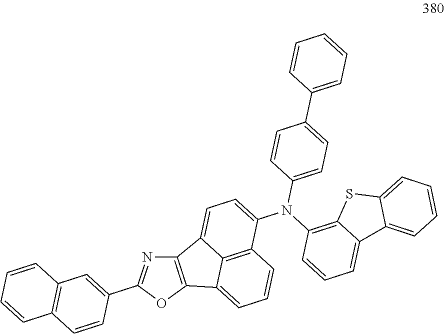 [SMILES: c1ccc(-c2ccc(N(c3ccc4c5c(cccc35)-c3oc(-c5ccc6ccccc6c5)nc3-4)c3cccc4c3sc3ccccc34)cc2)cc1]

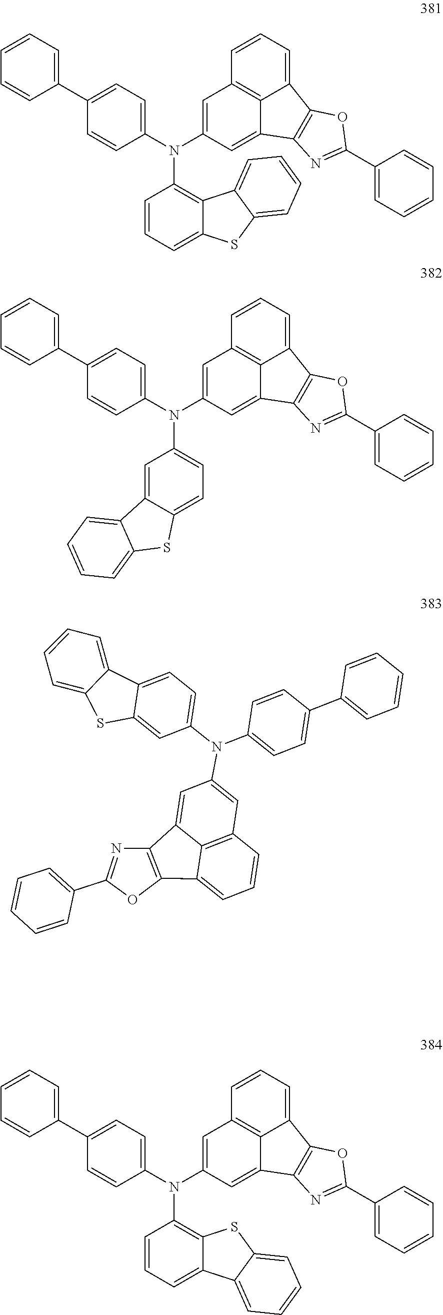 [SMILES: c1ccc(-c2ccc(N(c3cc4c5c(cccc5c3)-c3oc(-c5ccccc5)nc3-4)c3cccc4sc5ccccc5c34)cc2)cc1 ; c1ccc(-c2ccc(N(c3cc4c5c(cccc5c3)-c3oc(-c5ccccc5)nc3-4)c3ccc4sc5ccccc5c4c3)cc2)cc1 ; c1ccc(-c2ccc(N(c3cc4c5c(cccc5c3)-c3oc(-c5ccccc5)nc3-4)c3ccc4c(c3)sc3ccccc34)cc2)cc1 ; c1ccc(-c2ccc(N(c3cc4c5c(cccc5c3)-c3oc(-c5ccccc5)nc3-4)c3cccc4c3sc3ccccc34)cc2)cc1]

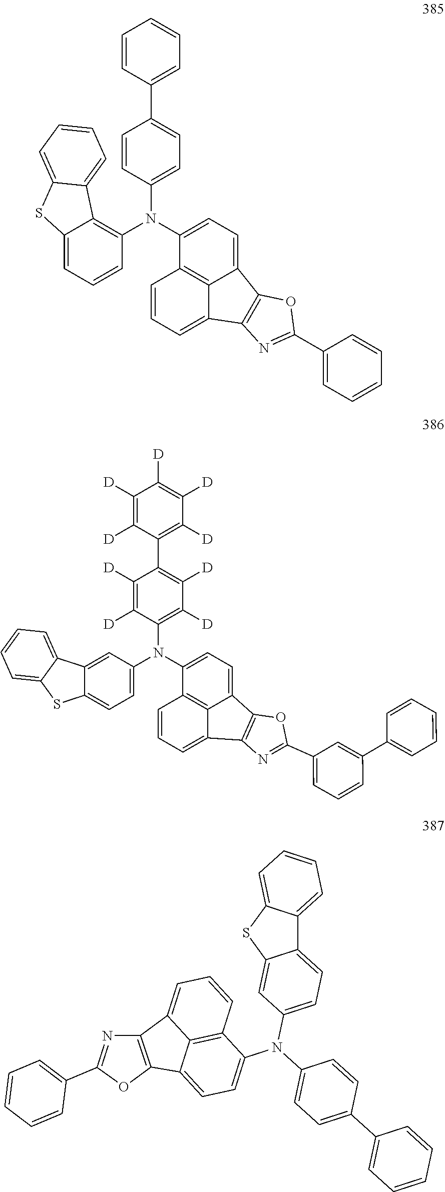 [SMILES: c1ccc(-c2ccc(N(c3ccc4c5c(cccc35)-c3nc(-c5ccccc5)oc3-4)c3cccc4sc5ccccc5c34)cc2)cc1 ; [2H]c1c([2H])c([2H])c(-c2c([2H])c([2H])c(N(c3ccc4sc5ccccc5c4c3)c3ccc4c5c(cccc35)-c3nc(-c5cccc(-c6ccccc6)c5)oc3-4)c([2H])c2[2H])c([2H])c1[2H] ; c1ccc(-c2ccc(N(c3ccc4c(c3)sc3ccccc34)c3ccc4c5c(cccc35)-c3nc(-c5ccccc5)oc3-4)cc2)cc1]

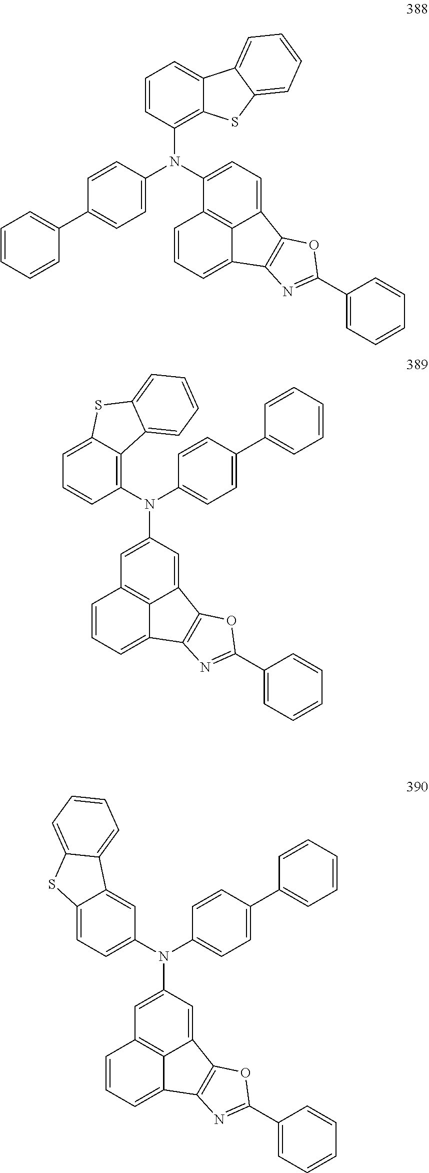 [SMILES: c1ccc(-c2ccc(N(c3ccc4c5c(cccc35)-c3nc(-c5ccccc5)oc3-4)c3cccc4c3sc3ccccc34)cc2)cc1 ; c1ccc(-c2ccc(N(c3cc4c5c(cccc5c3)-c3nc(-c5ccccc5)oc3-4)c3cccc4sc5ccccc5c34)cc2)cc1 ; c1ccc(-c2ccc(N(c3cc4c5c(cccc5c3)-c3nc(-c5ccccc5)oc3-4)c3ccc4sc5ccccc5c4c3)cc2)cc1]

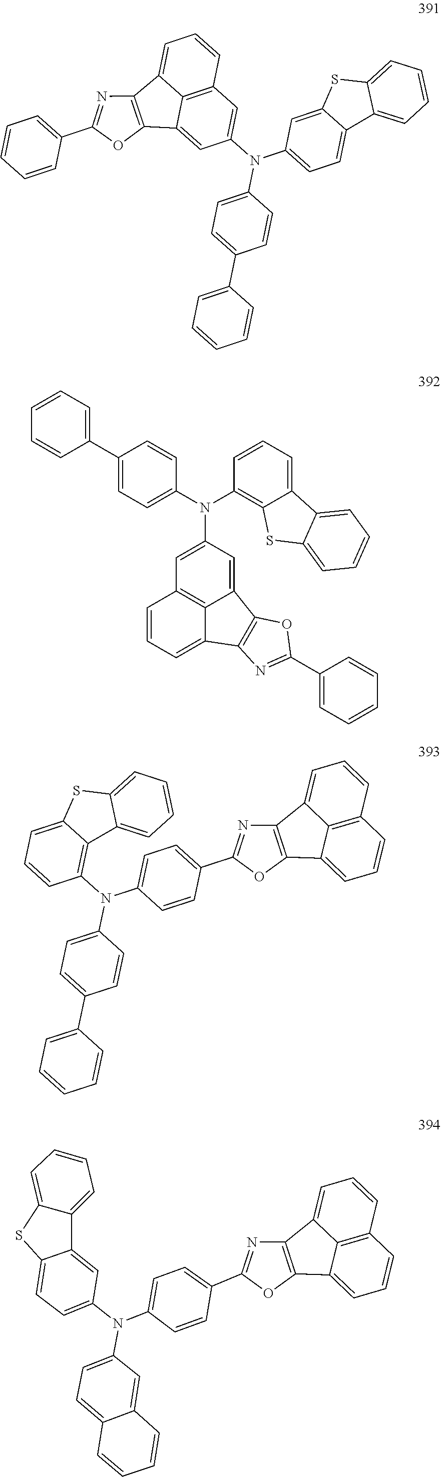

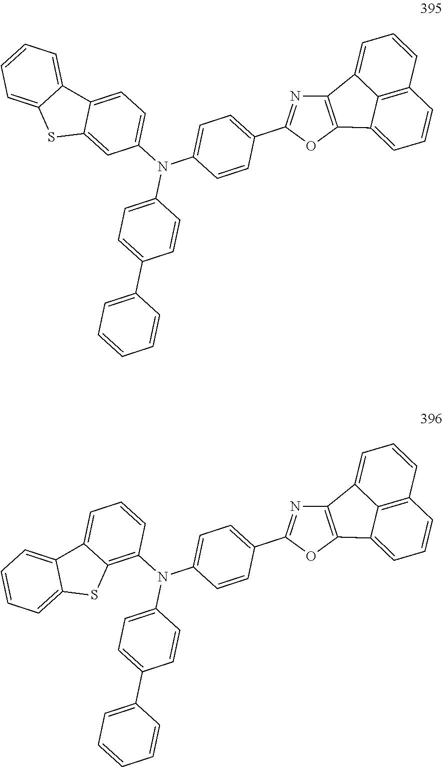 [SMILES: c1ccc(-c2ccc(N(c3ccc(-c4nc5c(o4)-c4cccc6cccc-5c46)cc3)c3ccc4c(c3)sc3ccccc34)cc2)cc1 ; c1ccc(-c2ccc(N(c3ccc(-c4nc5c(o4)-c4cccc6cccc-5c46)cc3)c3cccc4c3sc3ccccc34)cc2)cc1]

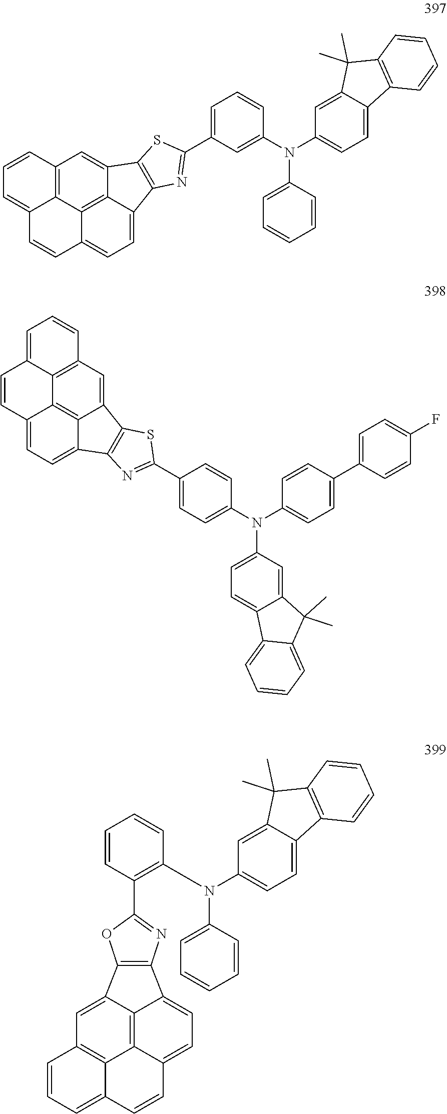 [SMILES: CC1(C)c2ccccc2-c2ccc(N(c3ccccc3)c3cccc(-c4nc5c(s4)-c4cc6cccc7ccc8ccc-5c4c8c76)c3)cc21 ; CC1(C)c2ccccc2-c2ccc(N(c3ccc(-c4ccc(F)cc4)cc3)c3ccc(-c4nc5c(s4)-c4cc6cccc7ccc8ccc-5c4c8c76)cc3)cc21 ; CC1(C)c2ccccc2-c2ccc(N(c3ccccc3)c3ccccc3-c3nc4c(o3)-c3cc5cccc6ccc7ccc-4c3c7c65)cc21]

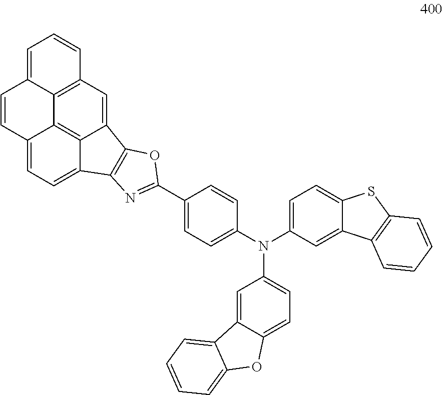 [SMILES: c1cc2ccc3ccc4c5c(cc(c1)c2c35)-c1oc(-c2ccc(N(c3ccc5oc6ccccc6c5c3)c3ccc5sc6ccccc6c5c3)cc2)nc1-4]

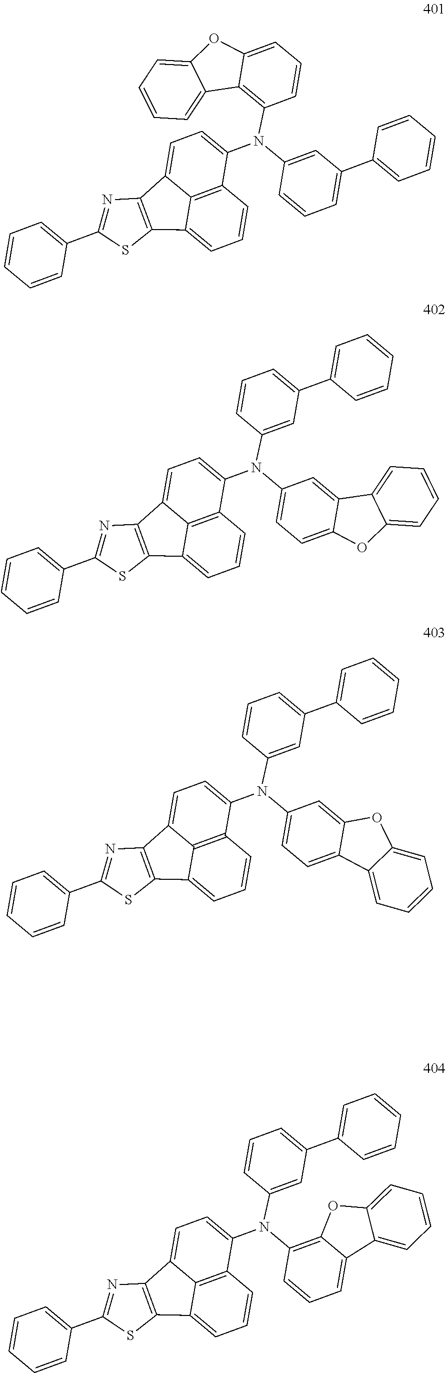 [SMILES: c1ccc(-c2cccc(N(c3ccc4c5c(cccc35)-c3sc(-c5ccccc5)nc3-4)c3cccc4oc5ccccc5c34)c2)cc1 ; c1ccc(-c2cccc(N(c3ccc4oc5ccccc5c4c3)c3ccc4c5c(cccc35)-c3sc(-c5ccccc5)nc3-4)c2)cc1 ; c1ccc(-c2cccc(N(c3ccc4c(c3)oc3ccccc34)c3ccc4c5c(cccc35)-c3sc(-c5ccccc5)nc3-4)c2)cc1 ; c1ccc(-c2cccc(N(c3ccc4c5c(cccc35)-c3sc(-c5ccccc5)nc3-4)c3cccc4c3oc3ccccc34)c2)cc1]

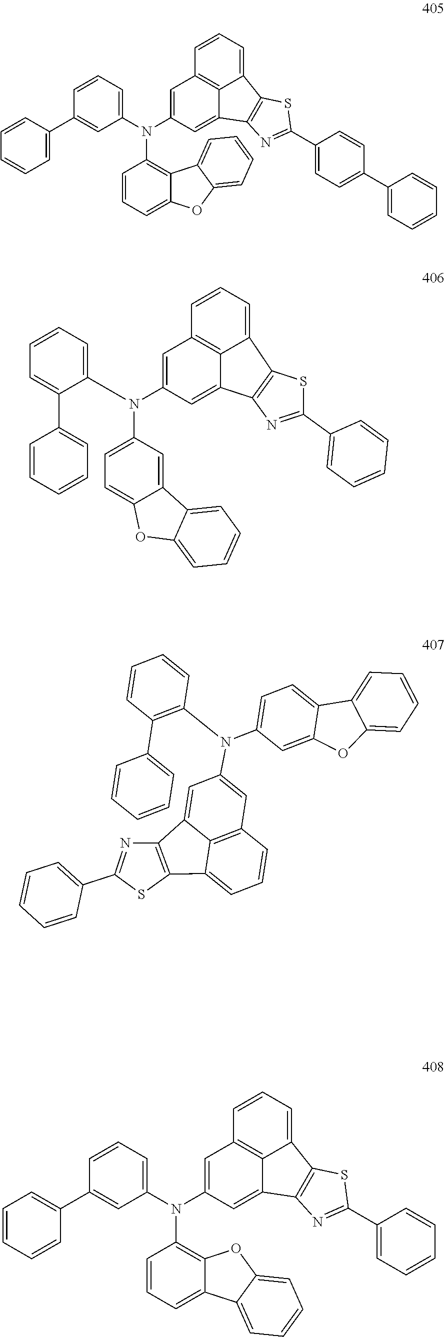 [SMILES: c1ccc(-c2ccc(-c3nc4c(s3)-c3cccc5cc(N(c6cccc(-c7ccccc7)c6)c6cccc7oc8ccccc8c67)cc-4c35)cc2)cc1 ; c1ccc(-c2nc3c(s2)-c2cccc4cc(N(c5ccc6oc7ccccc7c6c5)c5ccccc5-c5ccccc5)cc-3c24)cc1 ; c1ccc(-c2nc3c(s2)-c2cccc4cc(N(c5ccc6c(c5)oc5ccccc56)c5ccccc5-c5ccccc5)cc-3c24)cc1 ; c1ccc(-c2cccc(N(c3cc4c5c(cccc5c3)-c3sc(-c5ccccc5)nc3-4)c3cccc4c3oc3ccccc34)c2)cc1]

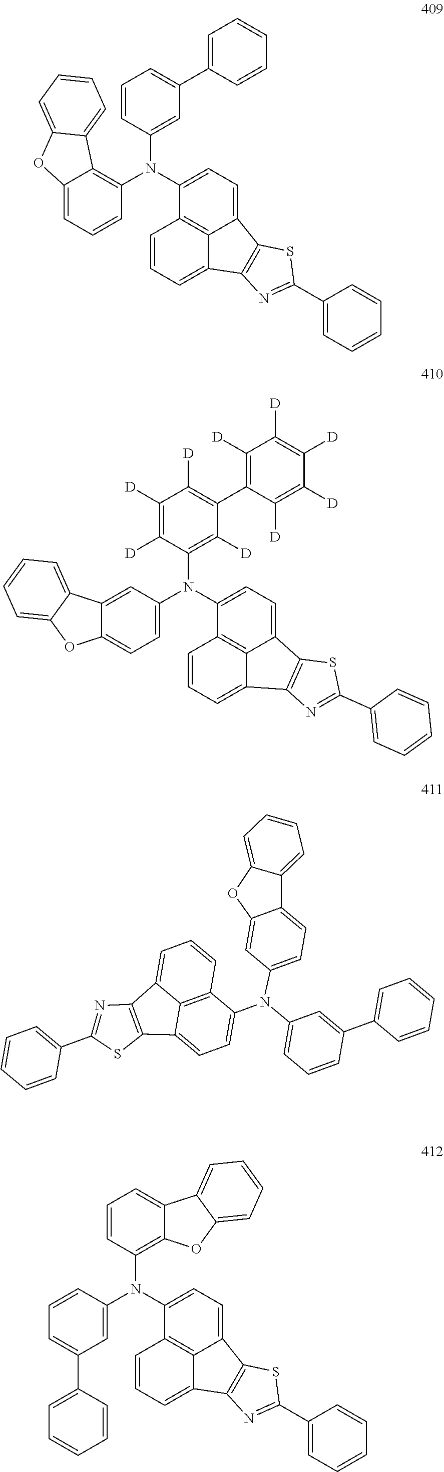 [SMILES: c1ccc(-c2cccc(N(c3ccc4c5c(cccc35)-c3nc(-c5ccccc5)sc3-4)c3cccc4oc5ccccc5c34)c2)cc1 ; [2H]c1c([2H])c([2H])c(-c2c([2H])c([2H])c([2H])c(N(c3ccc4oc5ccccc5c4c3)c3ccc4c5c(cccc35)-c3nc(-c5ccccc5)sc3-4)c2[2H])c([2H])c1[2H] ; c1ccc(-c2cccc(N(c3ccc4c(c3)oc3ccccc34)c3ccc4c5c(cccc35)-c3nc(-c5ccccc5)sc3-4)c2)cc1 ; c1ccc(-c2cccc(N(c3ccc4c5c(cccc35)-c3nc(-c5ccccc5)sc3-4)c3cccc4c3oc3ccccc34)c2)cc1]

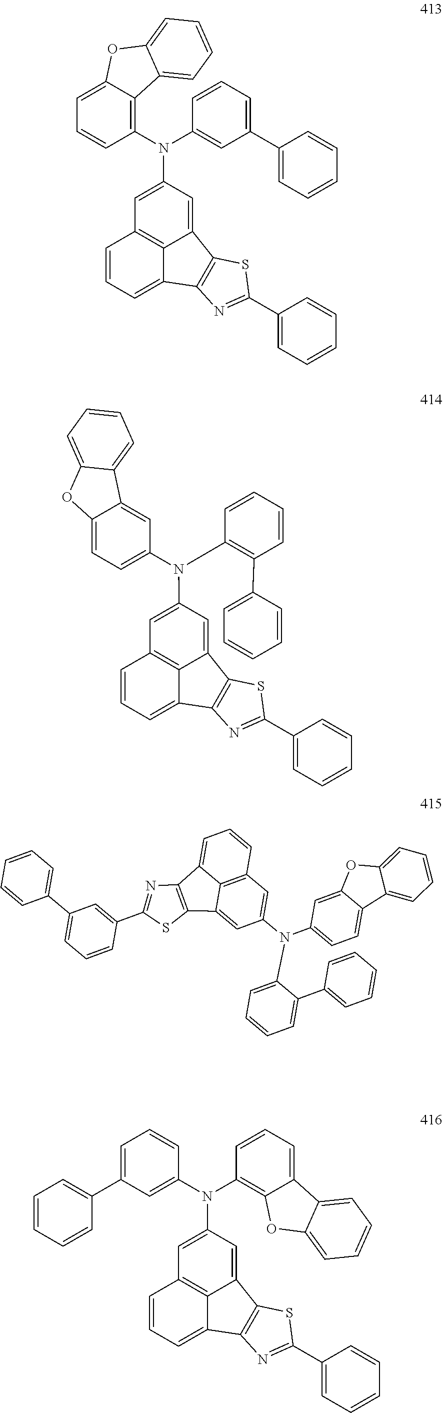 [SMILES: c1ccc(-c2cccc(N(c3cc4c5c(cccc5c3)-c3nc(-c5ccccc5)sc3-4)c3cccc4oc5ccccc5c34)c2)cc1 ; c1ccc(-c2nc3c(s2)-c2cc(N(c4ccc5oc6ccccc6c5c4)c4ccccc4-c4ccccc4)cc4cccc-3c24)cc1 ; c1ccc(-c2cccc(-c3nc4c(s3)-c3cc(N(c5ccc6c(c5)oc5ccccc56)c5ccccc5-c5ccccc5)cc5cccc-4c35)c2)cc1 ; c1ccc(-c2cccc(N(c3cc4c5c(cccc5c3)-c3nc(-c5ccccc5)sc3-4)c3cccc4c3oc3ccccc34)c2)cc1]

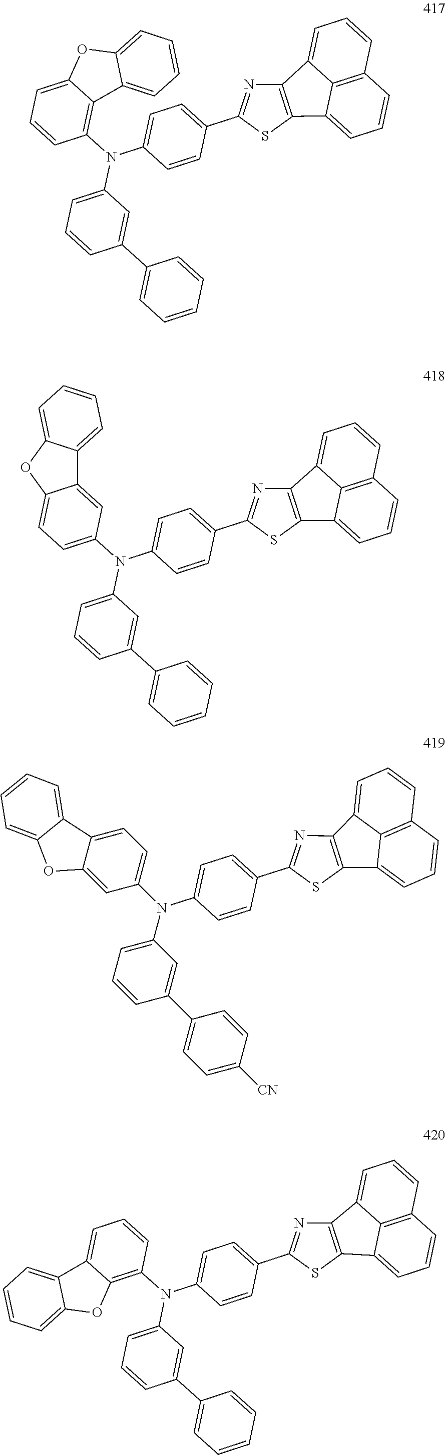 [SMILES: c1ccc(-c2cccc(N(c3ccc(-c4nc5c(s4)-c4cccc6cccc-5c46)cc3)c3cccc4oc5ccccc5c34)c2)cc1 ; c1ccc(-c2cccc(N(c3ccc(-c4nc5c(s4)-c4cccc6cccc-5c46)cc3)c3ccc4oc5ccccc5c4c3)c2)cc1 ; N#Cc1ccc(-c2cccc(N(c3ccc(-c4nc5c(s4)-c4cccc6cccc-5c46)cc3)c3ccc4c(c3)oc3ccccc34)c2)cc1 ; c1ccc(-c2cccc(N(c3ccc(-c4nc5c(s4)-c4cccc6cccc-5c46)cc3)c3cccc4c3oc3ccccc34)c2)cc1]

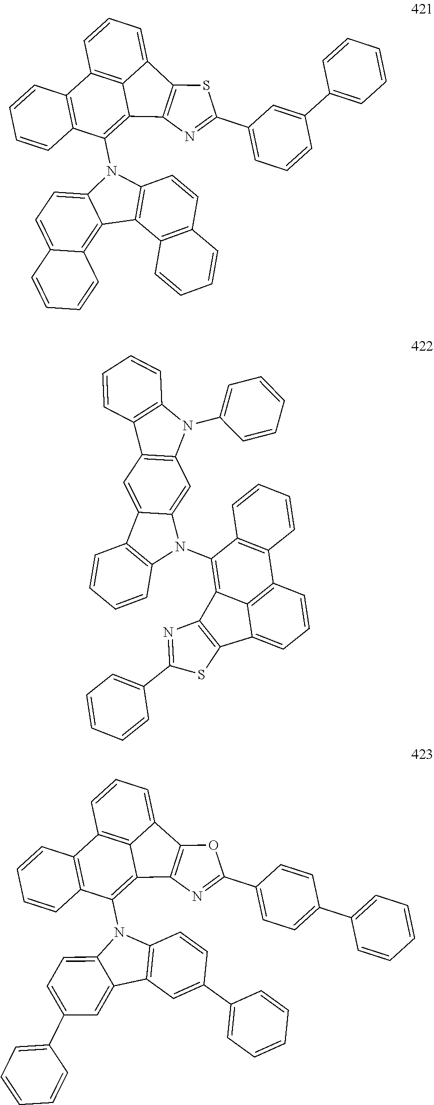

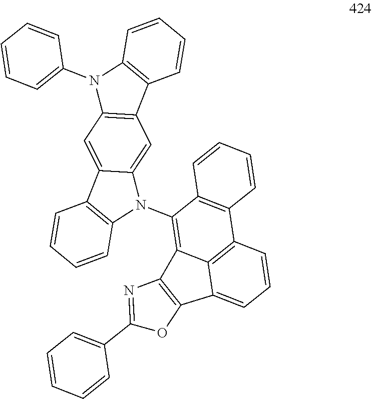 [SMILES: c1ccc(-c2nc3c(o2)-c2cccc4c2c-3c(-n2c3ccccc3c3cc5c(cc32)c2ccccc2n5-c2ccccc2)c2ccccc24)cc1]

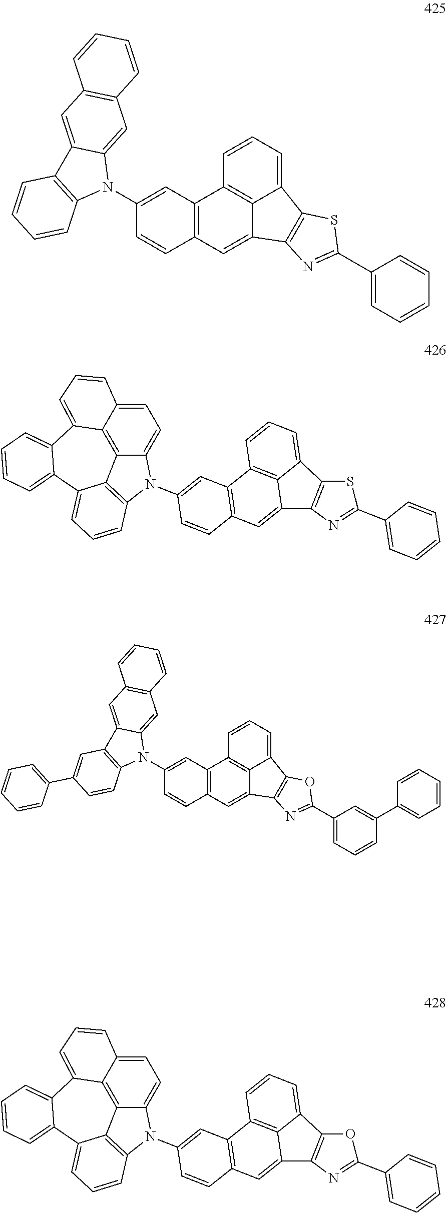

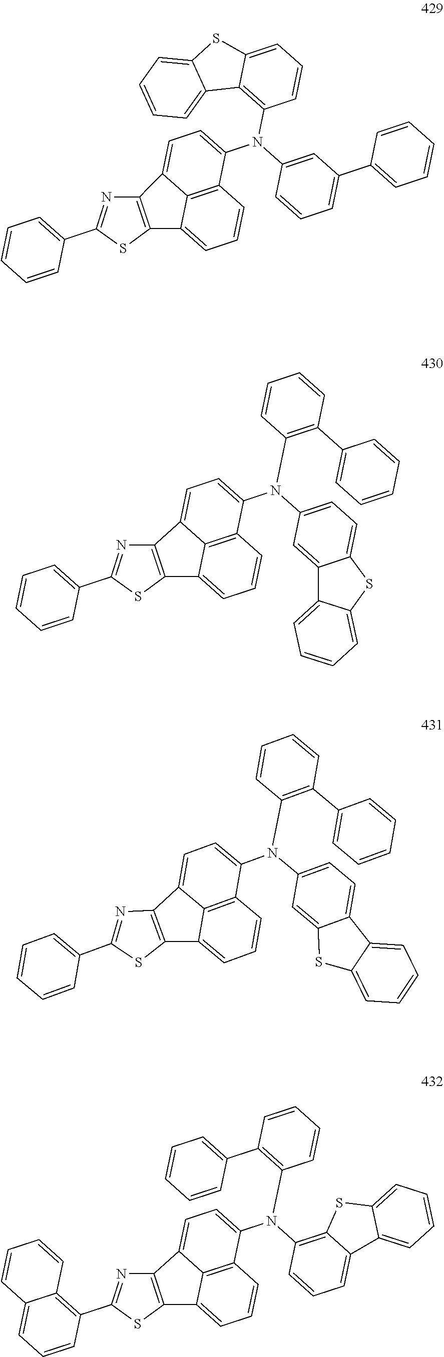 [SMILES: c1ccc(-c2cccc(N(c3ccc4c5c(cccc35)-c3sc(-c5ccccc5)nc3-4)c3cccc4sc5ccccc5c34)c2)cc1 ; c1ccc(-c2nc3c(s2)-c2cccc4c(N(c5ccc6sc7ccccc7c6c5)c5ccccc5-c5ccccc5)ccc-3c24)cc1 ; c1ccc(-c2nc3c(s2)-c2cccc4c(N(c5ccc6c(c5)sc5ccccc56)c5ccccc5-c5ccccc5)ccc-3c24)cc1 ; c1ccc(-c2ccccc2N(c2ccc3c4c(cccc24)-c2sc(-c4cccc5ccccc45)nc2-3)c2cccc3c2sc2ccccc23)cc1]

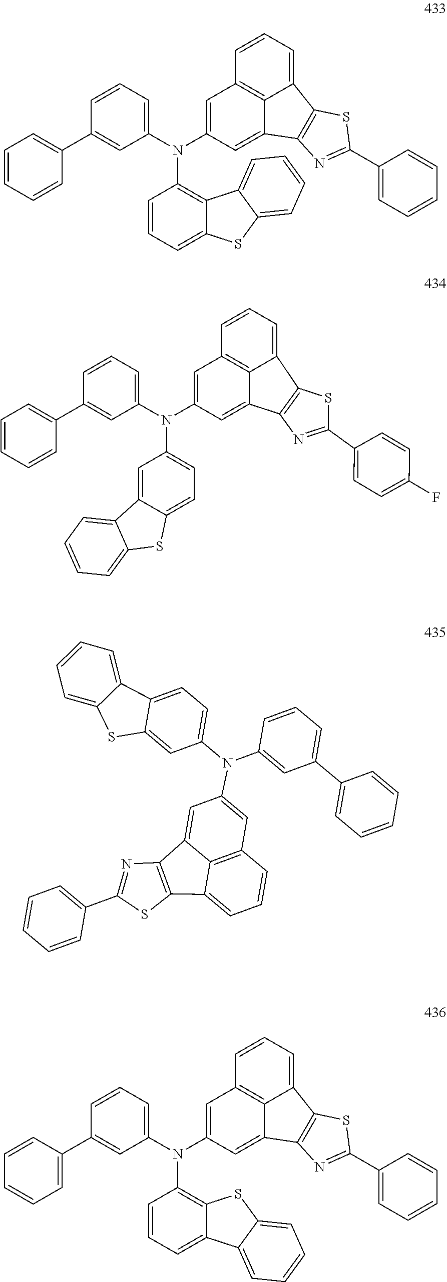 [SMILES: c1ccc(-c2cccc(N(c3cc4c5c(cccc5c3)-c3sc(-c5ccccc5)nc3-4)c3cccc4sc5ccccc5c34)c2)cc1 ; Fc1ccc(-c2nc3c(s2)-c2cccc4cc(N(c5cccc(-c6ccccc6)c5)c5ccc6sc7ccccc7c6c5)cc-3c24)cc1 ; c1ccc(-c2cccc(N(c3cc4c5c(cccc5c3)-c3sc(-c5ccccc5)nc3-4)c3ccc4c(c3)sc3ccccc34)c2)cc1 ; c1ccc(-c2cccc(N(c3cc4c5c(cccc5c3)-c3sc(-c5ccccc5)nc3-4)c3cccc4c3sc3ccccc34)c2)cc1]

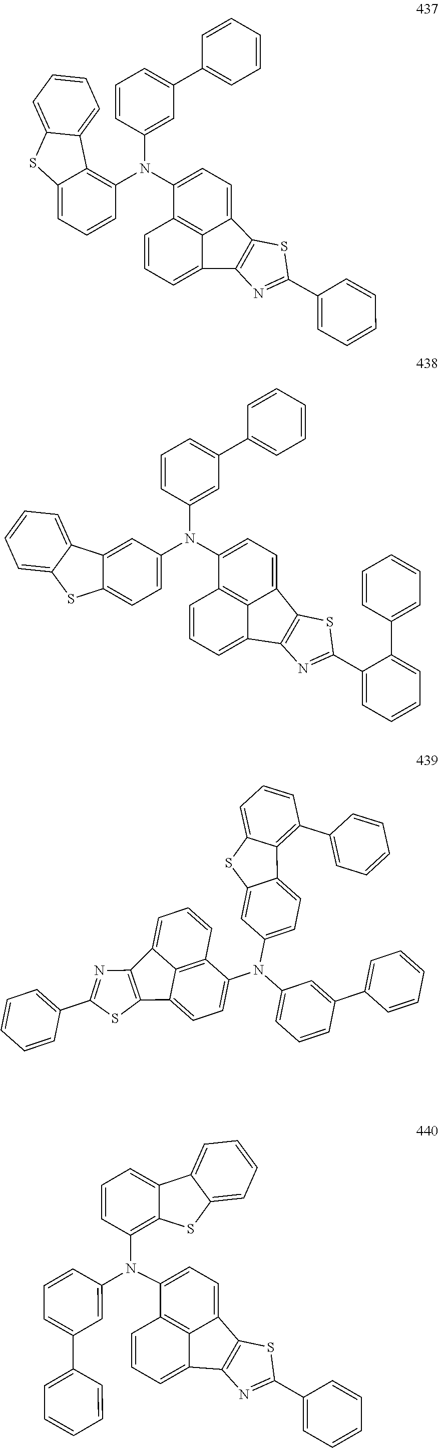 [SMILES: c1ccc(-c2cccc(N(c3ccc4c5c(cccc35)-c3nc(-c5ccccc5)sc3-4)c3cccc4sc5ccccc5c34)c2)cc1 ; c1ccc(-c2cccc(N(c3ccc4sc5ccccc5c4c3)c3ccc4c5c(cccc35)-c3nc(-c5ccccc5-c5ccccc5)sc3-4)c2)cc1 ; c1ccc(-c2cccc(N(c3ccc4c(c3)sc3cccc(-c5ccccc5)c34)c3ccc4c5c(cccc35)-c3nc(-c5ccccc5)sc3-4)c2)cc1 ; c1ccc(-c2cccc(N(c3ccc4c5c(cccc35)-c3nc(-c5ccccc5)sc3-4)c3cccc4c3sc3ccccc34)c2)cc1]

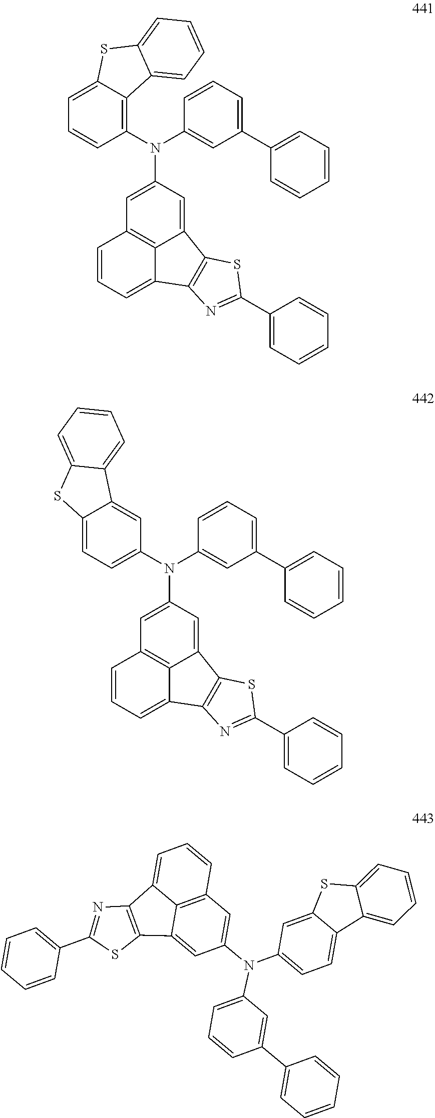 [SMILES: c1ccc(-c2cccc(N(c3cc4c5c(cccc5c3)-c3nc(-c5ccccc5)sc3-4)c3cccc4sc5ccccc5c34)c2)cc1 ; c1ccc(-c2cccc(N(c3cc4c5c(cccc5c3)-c3nc(-c5ccccc5)sc3-4)c3ccc4sc5ccccc5c4c3)c2)cc1 ; c1ccc(-c2cccc(N(c3cc4c5c(cccc5c3)-c3nc(-c5ccccc5)sc3-4)c3ccc4c(c3)sc3ccccc34)c2)cc1]

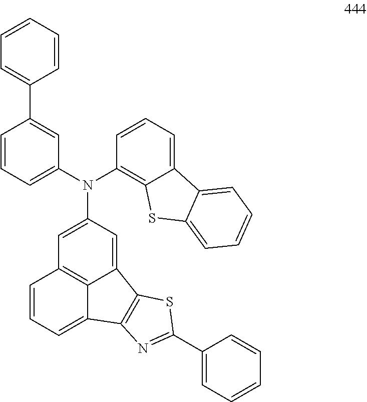 [SMILES: c1ccc(-c2cccc(N(c3cc4c5c(cccc5c3)-c3nc(-c5ccccc5)sc3-4)c3cccc4c3sc3ccccc34)c2)cc1]

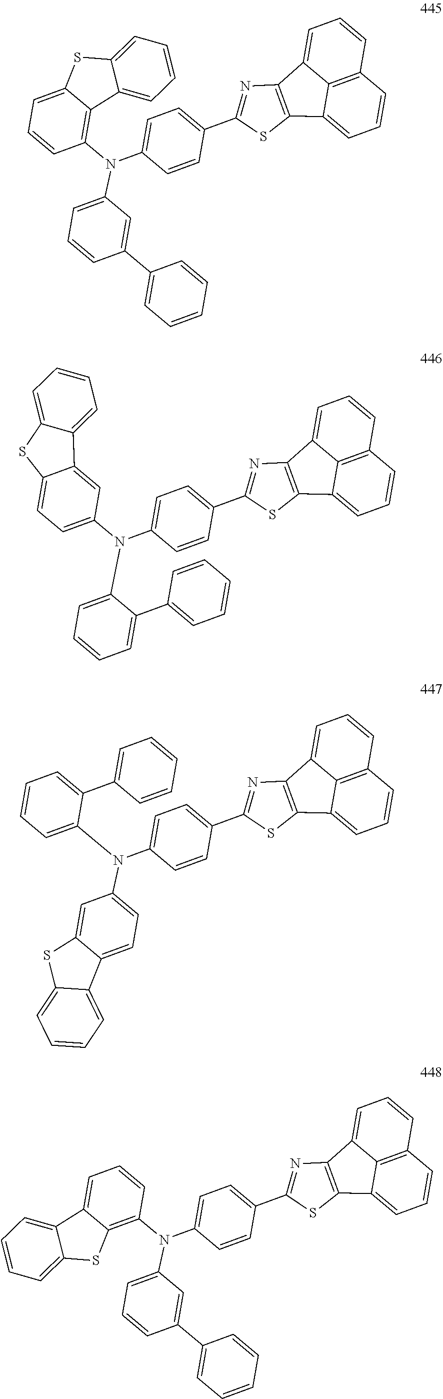 [SMILES: c1ccc(-c2cccc(N(c3ccc(-c4nc5c(s4)-c4cccc6cccc-5c46)cc3)c3cccc4sc5ccccc5c34)c2)cc1 ; c1ccc(-c2ccccc2N(c2ccc(-c3nc4c(s3)-c3cccc5cccc-4c35)cc2)c2ccc3sc4ccccc4c3c2)cc1 ; c1ccc(-c2ccccc2N(c2ccc(-c3nc4c(s3)-c3cccc5cccc-4c35)cc2)c2ccc3c(c2)sc2ccccc23)cc1 ; c1ccc(-c2cccc(N(c3ccc(-c4nc5c(s4)-c4cccc6cccc-5c46)cc3)c3cccc4c3sc3ccccc34)c2)cc1]

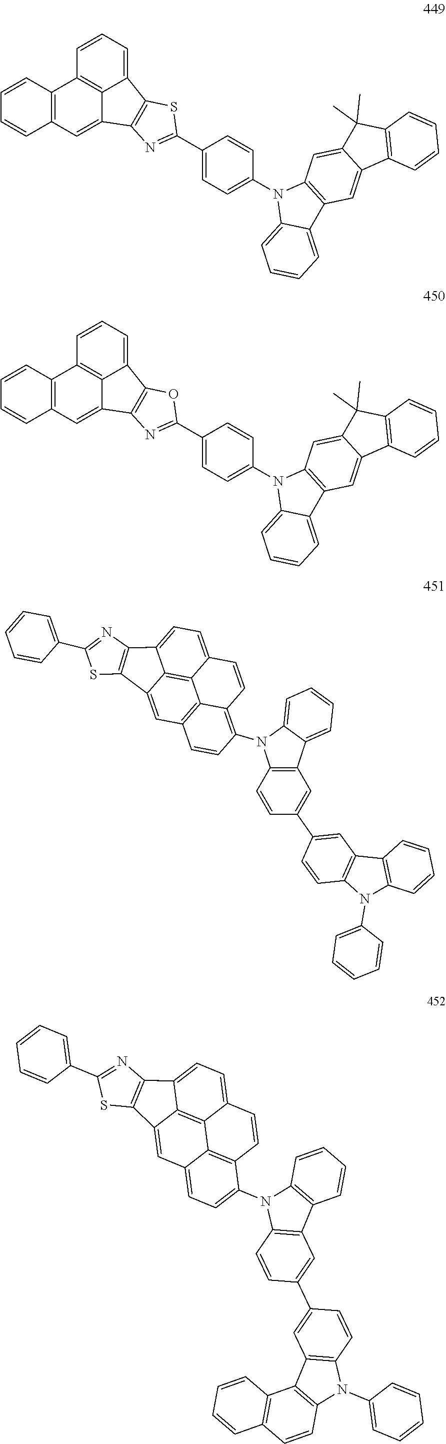 [SMILES: CC1(C)c2ccccc2-c2cc3c4ccccc4n(-c4ccc(-c5nc6c(s5)-c5cccc7c5c-6cc5ccccc57)cc4)c3cc21 ; CC1(C)c2ccccc2-c2cc3c4ccccc4n(-c4ccc(-c5nc6c(o5)-c5cccc7c5c-6cc5ccccc57)cc4)c3cc21 ; c1ccc(-c2nc3c(s2)-c2cc4ccc(-n5c6ccccc6c6cc(-c7ccc8c(c7)c7ccccc7n8-c7ccccc7)ccc65)c5ccc6ccc-3c2c6c45)cc1 ; c1ccc(-c2nc3c(s2)-c2cc4ccc(-n5c6ccccc6c6cc(-c7ccc8c(c7)c7c9ccccc9ccc7n8-c7ccccc7)ccc65)c5ccc6ccc-3c2c6c45)cc1]

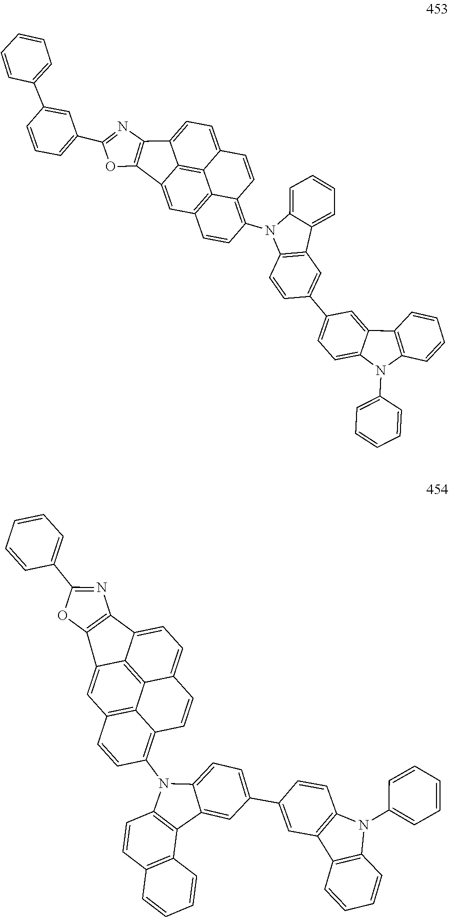 [SMILES: c1ccc(-c2cccc(-c3nc4c(o3)-c3cc5ccc(-n6c7ccccc7c7cc(-c8ccc9c(c8)c8ccccc8n9-c8ccccc8)ccc76)c6ccc7ccc-4c3c7c56)c2)cc1 ; c1ccc(-c2nc3c(o2)-c2cc4ccc(-n5c6ccc(-c7ccc8c(c7)c7ccccc7n8-c7ccccc7)cc6c6c7ccccc7ccc65)c5ccc6ccc-3c2c6c45)cc1]

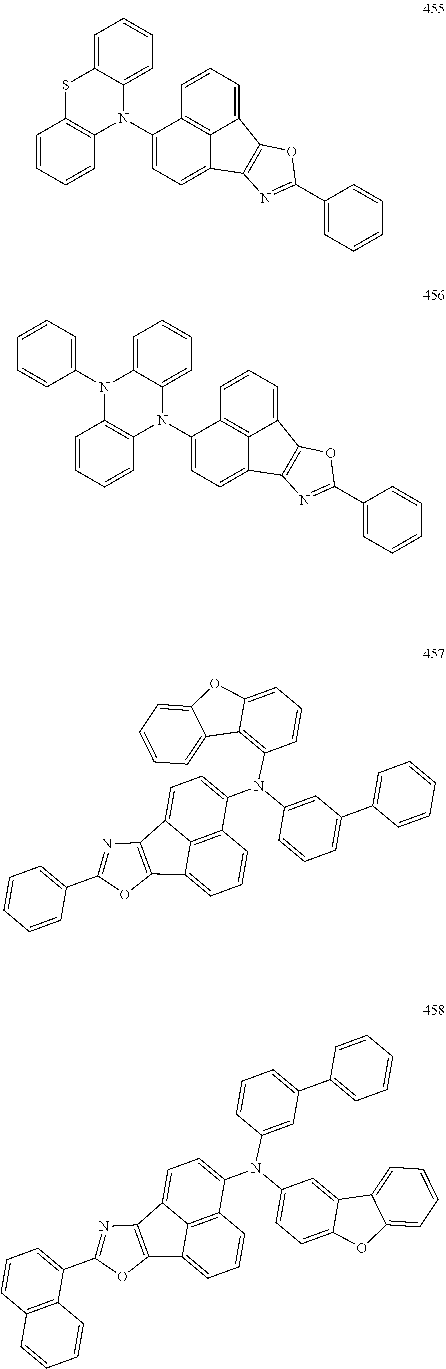 [SMILES: c1ccc(-c2nc3c(o2)-c2cccc4c(N5c6ccccc6Sc6ccccc65)ccc-3c24)cc1 ; c1ccc(-c2nc3c(o2)-c2cccc4c(N5c6ccccc6N(c6ccccc6)c6ccccc65)ccc-3c24)cc1 ; c1ccc(-c2cccc(N(c3ccc4c5c(cccc35)-c3oc(-c5ccccc5)nc3-4)c3cccc4oc5ccccc5c34)c2)cc1 ; c1ccc(-c2cccc(N(c3ccc4oc5ccccc5c4c3)c3ccc4c5c(cccc35)-c3oc(-c5cccc6ccccc56)nc3-4)c2)cc1]

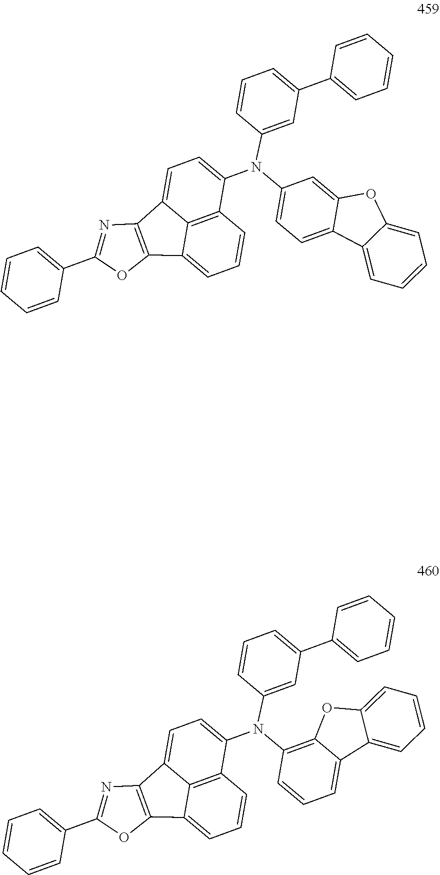 [SMILES: c1ccc(-c2cccc(N(c3ccc4c(c3)oc3ccccc34)c3ccc4c5c(cccc35)-c3oc(-c5ccccc5)nc3-4)c2)cc1 ; c1ccc(-c2cccc(N(c3ccc4c5c(cccc35)-c3oc(-c5ccccc5)nc3-4)c3cccc4c3oc3ccccc34)c2)cc1]

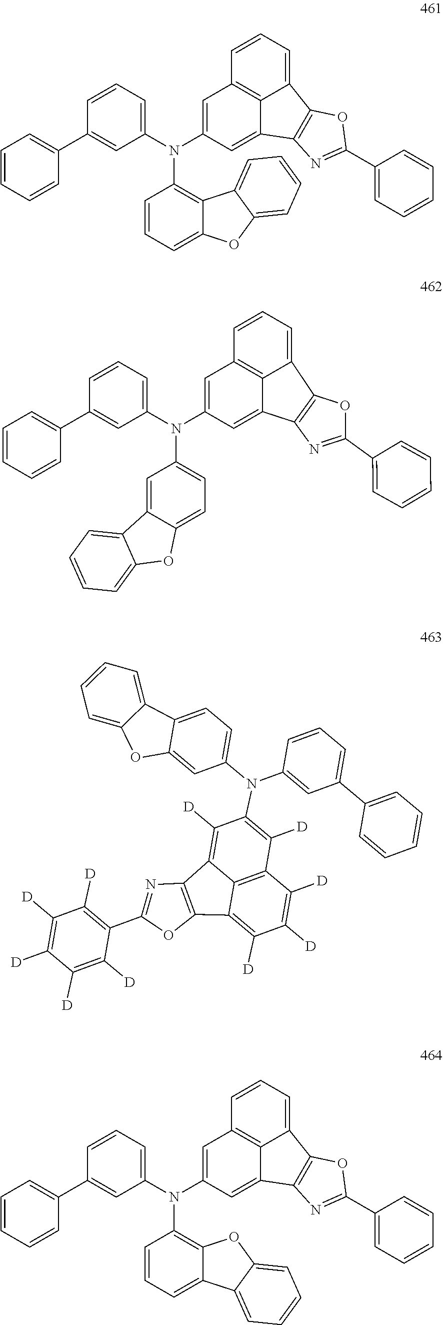 [SMILES: c1ccc(-c2cccc(N(c3cc4c5c(cccc5c3)-c3oc(-c5ccccc5)nc3-4)c3cccc4oc5ccccc5c34)c2)cc1 ; c1ccc(-c2cccc(N(c3cc4c5c(cccc5c3)-c3oc(-c5ccccc5)nc3-4)c3ccc4oc5ccccc5c4c3)c2)cc1 ; [2H]c1c([2H])c([2H])c(-c2nc3c(o2)-c2c([2H])c([2H])c([2H])c4c([2H])c(N(c5cccc(-c6ccccc6)c5)c5ccc6c(c5)oc5ccccc56)c([2H])c-3c24)c([2H])c1[2H] ; c1ccc(-c2cccc(N(c3cc4c5c(cccc5c3)-c3oc(-c5ccccc5)nc3-4)c3cccc4c3oc3ccccc34)c2)cc1]

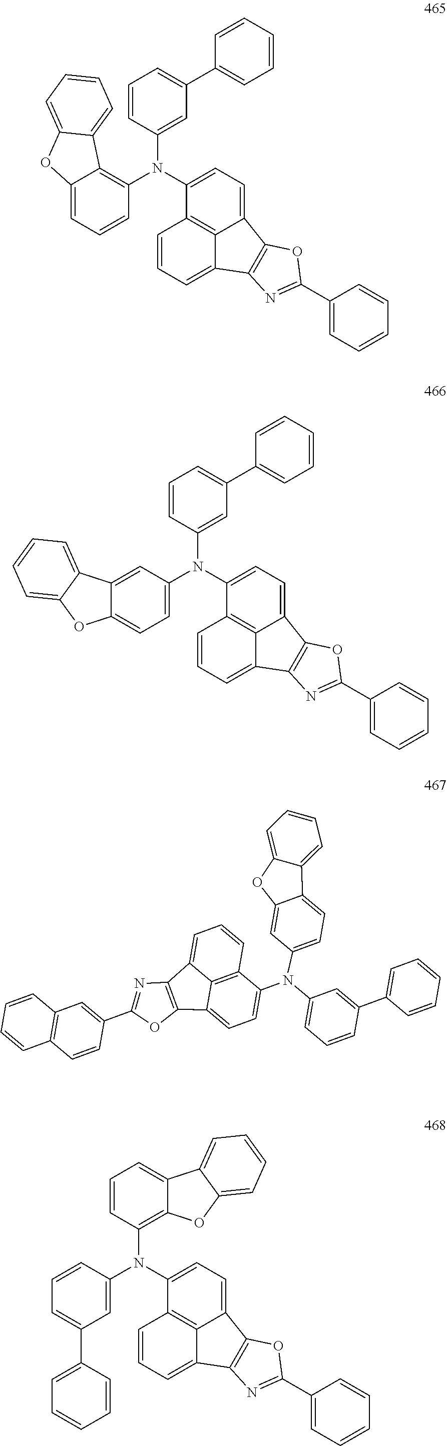

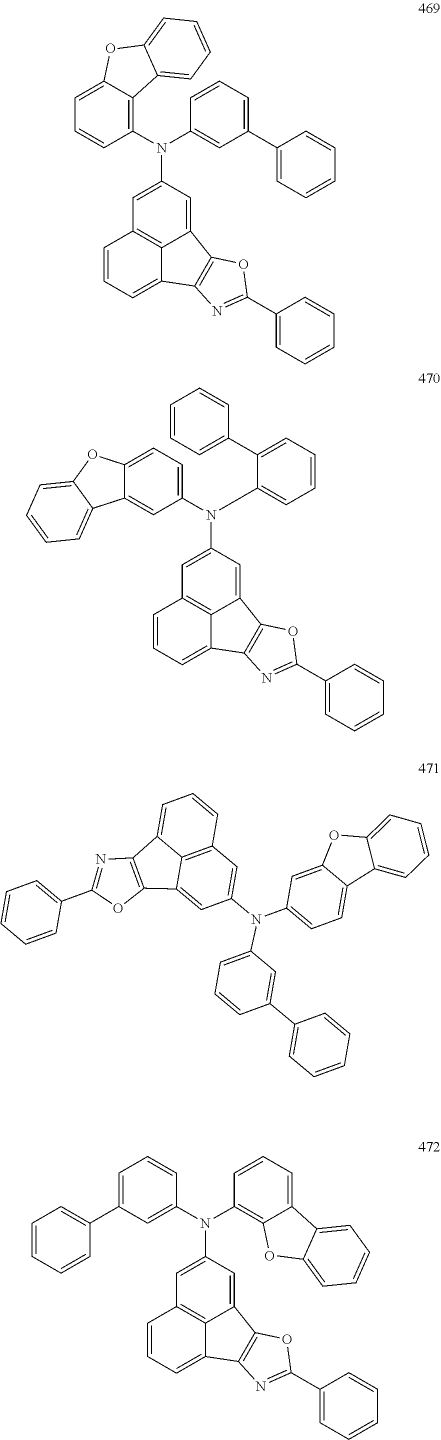 [SMILES: c1ccc(-c2cccc(N(c3cc4c5c(cccc5c3)-c3nc(-c5ccccc5)oc3-4)c3cccc4oc5ccccc5c34)c2)cc1 ; c1ccc(-c2nc3c(o2)-c2cc(N(c4ccc5oc6ccccc6c5c4)c4ccccc4-c4ccccc4)cc4cccc-3c24)cc1 ; c1ccc(-c2cccc(N(c3cc4c5c(cccc5c3)-c3nc(-c5ccccc5)oc3-4)c3ccc4c(c3)oc3ccccc34)c2)cc1 ; c1ccc(-c2cccc(N(c3cc4c5c(cccc5c3)-c3nc(-c5ccccc5)oc3-4)c3cccc4c3oc3ccccc34)c2)cc1]

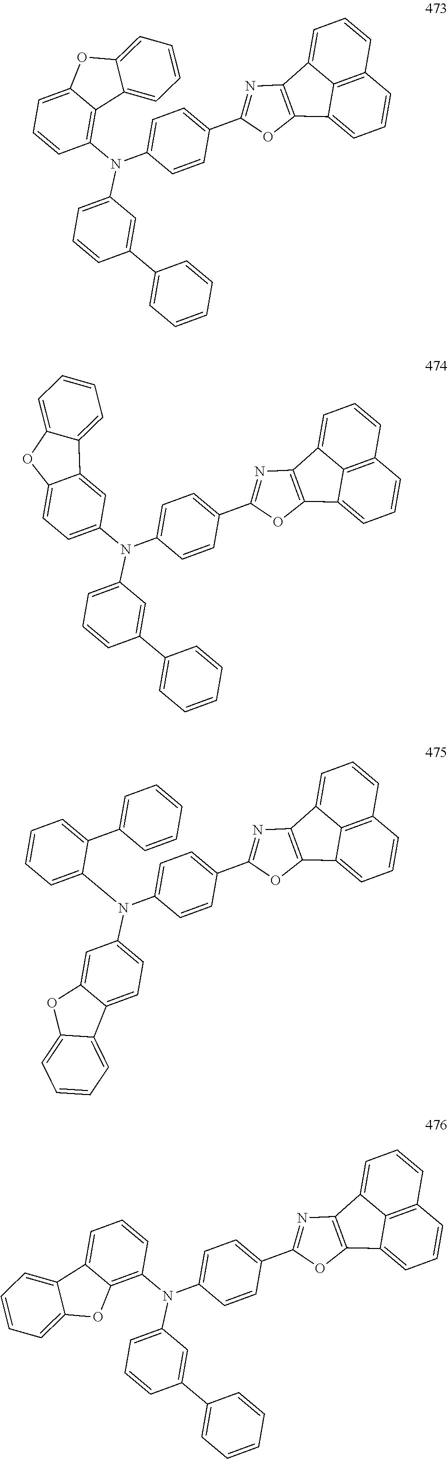 [SMILES: c1ccc(-c2cccc(N(c3ccc(-c4nc5c(o4)-c4cccc6cccc-5c46)cc3)c3cccc4oc5ccccc5c34)c2)cc1 ; c1ccc(-c2cccc(N(c3ccc(-c4nc5c(o4)-c4cccc6cccc-5c46)cc3)c3ccc4oc5ccccc5c4c3)c2)cc1 ; c1ccc(-c2ccccc2N(c2ccc(-c3nc4c(o3)-c3cccc5cccc-4c35)cc2)c2ccc3c(c2)oc2ccccc23)cc1 ; c1ccc(-c2cccc(N(c3ccc(-c4nc5c(o4)-c4cccc6cccc-5c46)cc3)c3cccc4c3oc3ccccc34)c2)cc1]

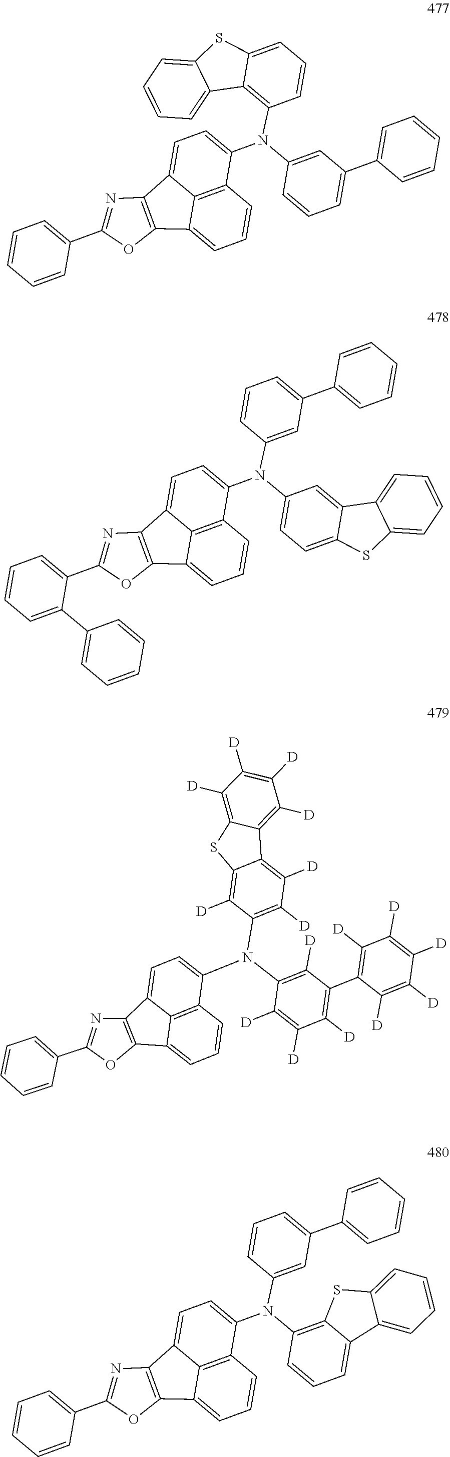 [SMILES: c1ccc(-c2cccc(N(c3ccc4c5c(cccc35)-c3oc(-c5ccccc5)nc3-4)c3cccc4sc5ccccc5c34)c2)cc1 ; c1ccc(-c2cccc(N(c3ccc4sc5ccccc5c4c3)c3ccc4c5c(cccc35)-c3oc(-c5ccccc5-c5ccccc5)nc3-4)c2)cc1 ; [2H]c1c([2H])c([2H])c(-c2c([2H])c([2H])c([2H])c(N(c3c([2H])c([2H])c4c(sc5c([2H])c([2H])c([2H])c([2H])c54)c3[2H])c3ccc4c5c(cccc35)-c3oc(-c5ccccc5)nc3-4)c2[2H])c([2H])c1[2H] ; c1ccc(-c2cccc(N(c3ccc4c5c(cccc35)-c3oc(-c5ccccc5)nc3-4)c3cccc4c3sc3ccccc34)c2)cc1]

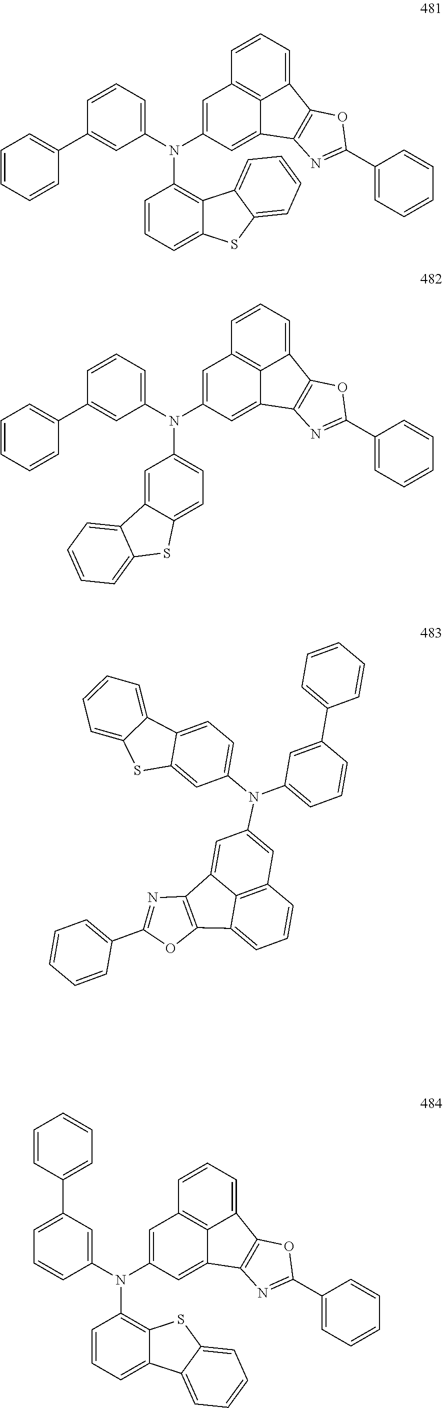 [SMILES: c1ccc(-c2cccc(N(c3cc4c5c(cccc5c3)-c3oc(-c5ccccc5)nc3-4)c3cccc4sc5ccccc5c34)c2)cc1 ; c1ccc(-c2cccc(N(c3cc4c5c(cccc5c3)-c3oc(-c5ccccc5)nc3-4)c3ccc4sc5ccccc5c4c3)c2)cc1 ; c1ccc(-c2cccc(N(c3cc4c5c(cccc5c3)-c3oc(-c5ccccc5)nc3-4)c3ccc4c(c3)sc3ccccc34)c2)cc1 ; c1ccc(-c2cccc(N(c3cc4c5c(cccc5c3)-c3oc(-c5ccccc5)nc3-4)c3cccc4c3sc3ccccc34)c2)cc1]

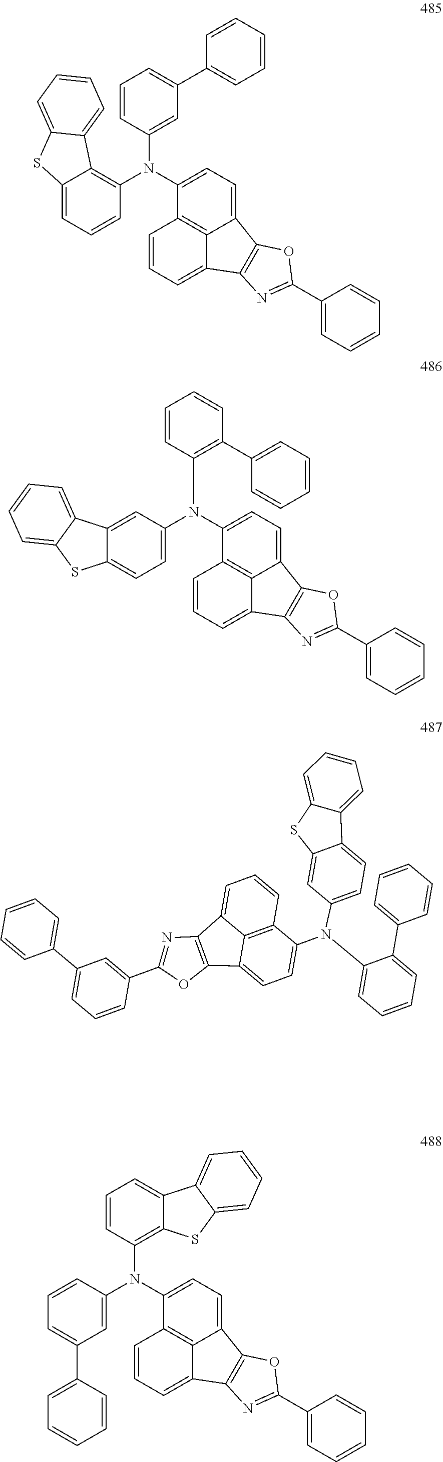 [SMILES: c1ccc(-c2cccc(N(c3ccc4c5c(cccc35)-c3nc(-c5ccccc5)oc3-4)c3cccc4sc5ccccc5c34)c2)cc1 ; c1ccc(-c2nc3c(o2)-c2ccc(N(c4ccc5sc6ccccc6c5c4)c4ccccc4-c4ccccc4)c4cccc-3c24)cc1 ; c1ccc(-c2cccc(-c3nc4c(o3)-c3ccc(N(c5ccc6c(c5)sc5ccccc56)c5ccccc5-c5ccccc5)c5cccc-4c35)c2)cc1 ; c1ccc(-c2cccc(N(c3ccc4c5c(cccc35)-c3nc(-c5ccccc5)oc3-4)c3cccc4c3sc3ccccc34)c2)cc1]

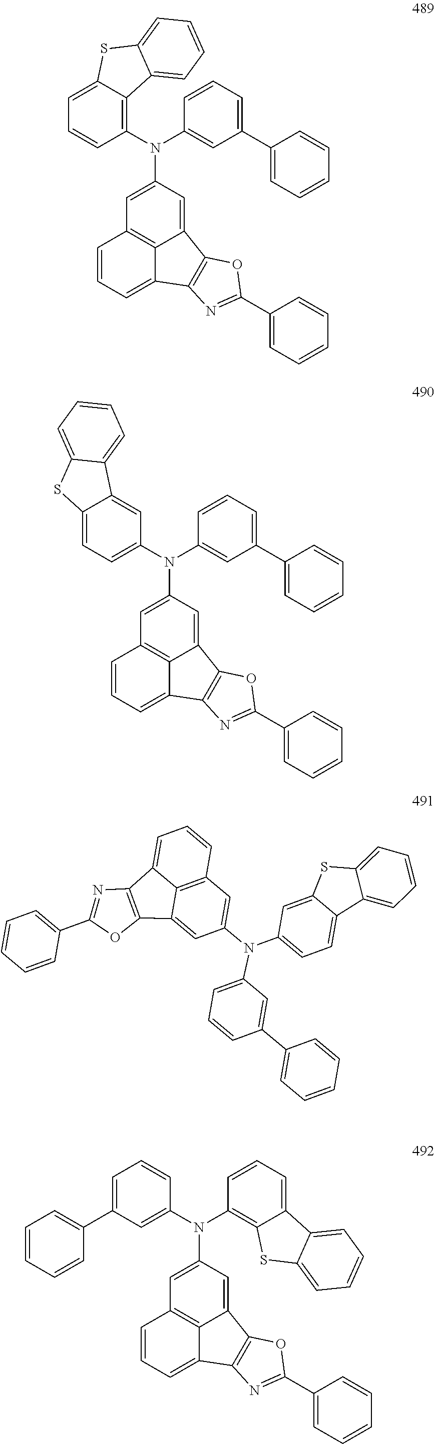

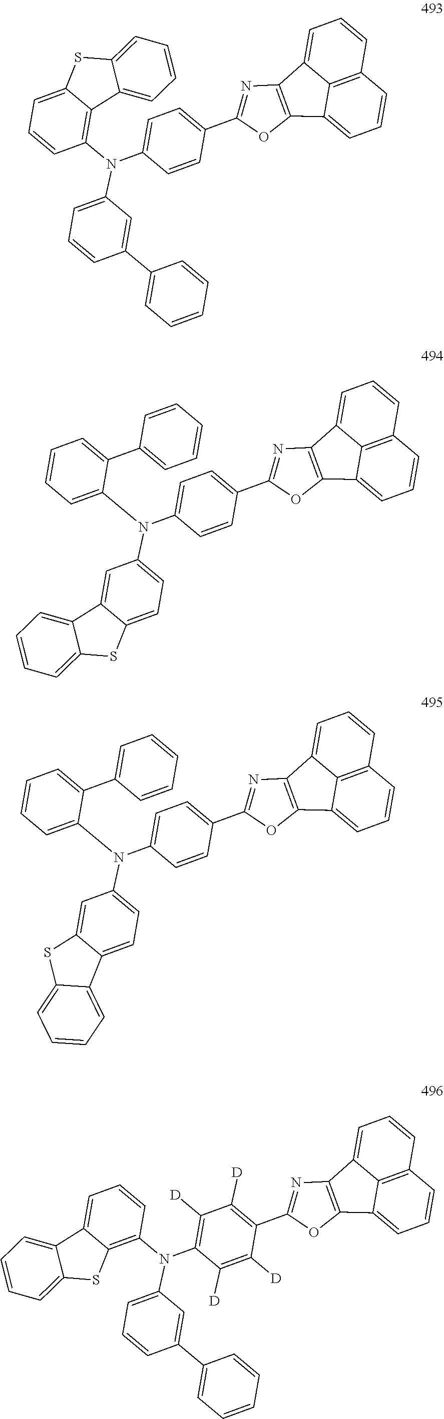 [SMILES: c1ccc(-c2cccc(N(c3ccc(-c4nc5c(o4)-c4cccc6cccc-5c46)cc3)c3cccc4sc5ccccc5c34)c2)cc1 ; c1ccc(-c2ccccc2N(c2ccc(-c3nc4c(o3)-c3cccc5cccc-4c35)cc2)c2ccc3sc4ccccc4c3c2)cc1 ; c1ccc(-c2ccccc2N(c2ccc(-c3nc4c(o3)-c3cccc5cccc-4c35)cc2)c2ccc3c(c2)sc2ccccc23)cc1 ; [2H]c1c([2H])c(N(c2cccc(-c3ccccc3)c2)c2cccc3c2sc2ccccc23)c([2H])c([2H])c1-c1nc2c(o1)-c1cccc3cccc-2c13]

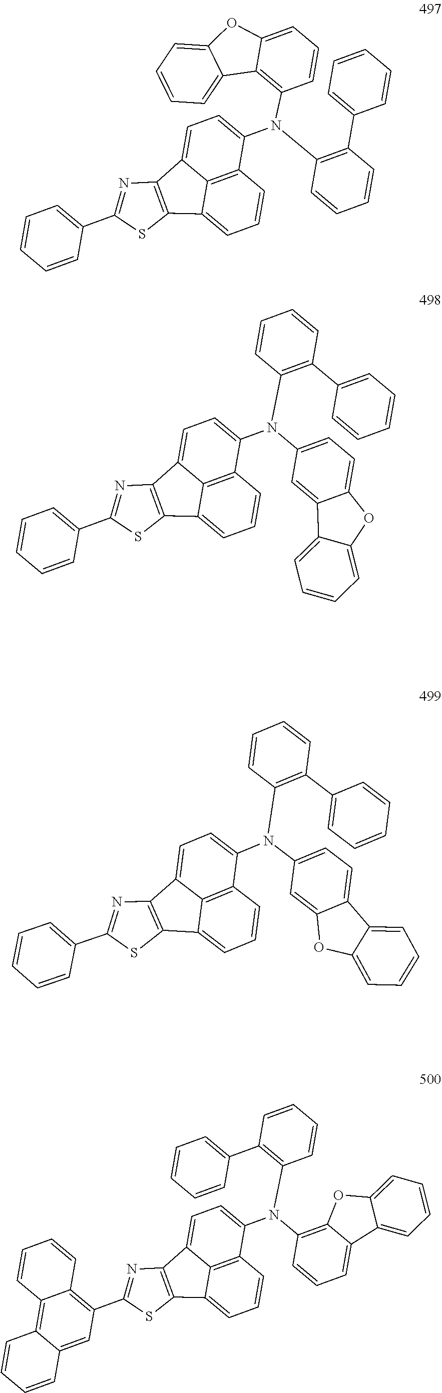 [SMILES: c1ccc(-c2nc3c(s2)-c2cccc4c(N(c5ccccc5-c5ccccc5)c5cccc6oc7ccccc7c56)ccc-3c24)cc1 ; c1ccc(-c2nc3c(s2)-c2cccc4c(N(c5ccc6oc7ccccc7c6c5)c5ccccc5-c5ccccc5)ccc-3c24)cc1 ; c1ccc(-c2nc3c(s2)-c2cccc4c(N(c5ccc6c(c5)oc5ccccc56)c5ccccc5-c5ccccc5)ccc-3c24)cc1 ; c1ccc(-c2ccccc2N(c2ccc3c4c(cccc24)-c2sc(-c4cc5ccccc5c5ccccc45)nc2-3)c2cccc3c2oc2ccccc23)cc1]

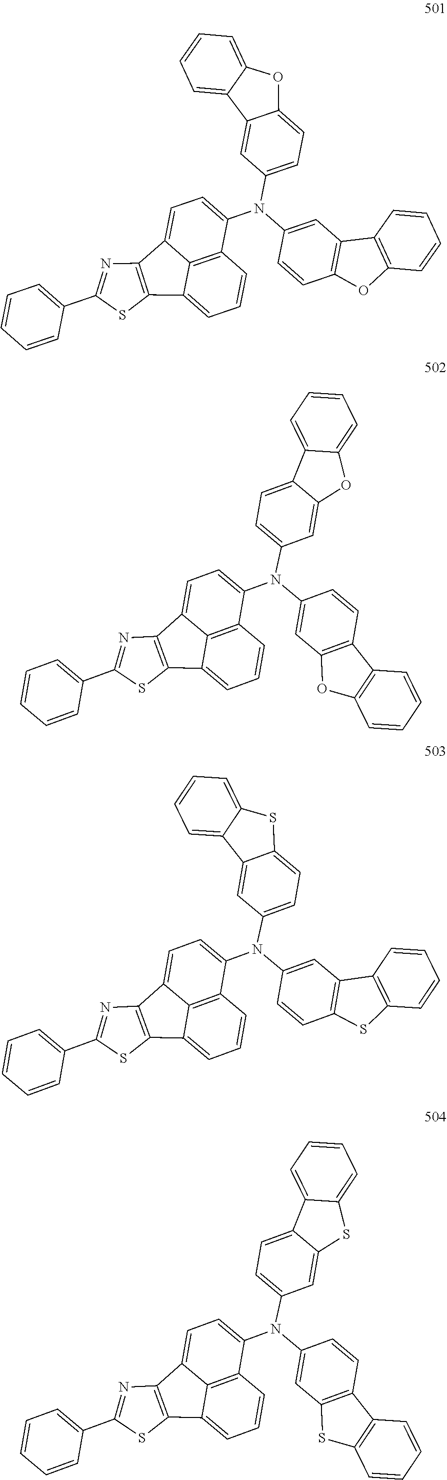 [SMILES: c1ccc(-c2nc3c(s2)-c2cccc4c(N(c5ccc6oc7ccccc7c6c5)c5ccc6oc7ccccc7c6c5)ccc-3c24)cc1 ; c1ccc(-c2nc3c(s2)-c2cccc4c(N(c5ccc6c(c5)oc5ccccc56)c5ccc6c(c5)oc5ccccc56)ccc-3c24)cc1 ; c1ccc(-c2nc3c(s2)-c2cccc4c(N(c5ccc6sc7ccccc7c6c5)c5ccc6sc7ccccc7c6c5)ccc-3c24)cc1 ; c1ccc(-c2nc3c(s2)-c2cccc4c(N(c5ccc6c(c5)sc5ccccc56)c5ccc6c(c5)sc5ccccc56)ccc-3c24)cc1]

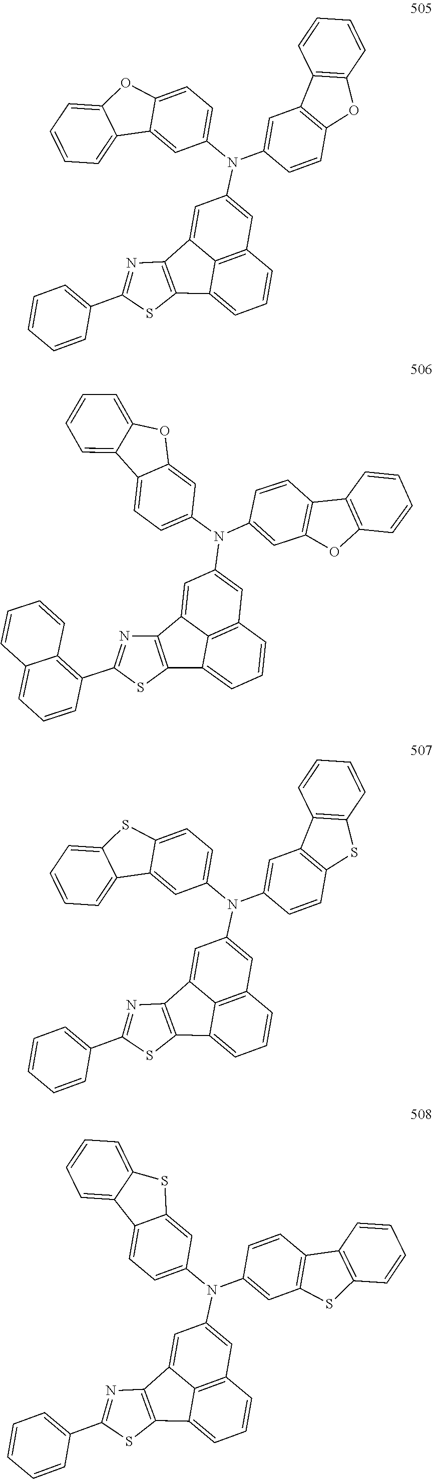 [SMILES: c1ccc(-c2nc3c(s2)-c2cccc4cc(N(c5ccc6oc7ccccc7c6c5)c5ccc6oc7ccccc7c6c5)cc-3c24)cc1 ; c1ccc2c(-c3nc4c(s3)-c3cccc5cc(N(c6ccc7c(c6)oc6ccccc67)c6ccc7c(c6)oc6ccccc67)cc-4c35)cccc2c1 ; c1ccc(-c2nc3c(s2)-c2cccc4cc(N(c5ccc6sc7ccccc7c6c5)c5ccc6sc7ccccc7c6c5)cc-3c24)cc1 ; c1ccc(-c2nc3c(s2)-c2cccc4cc(N(c5ccc6c(c5)sc5ccccc56)c5ccc6c(c5)sc5ccccc56)cc-3c24)cc1]

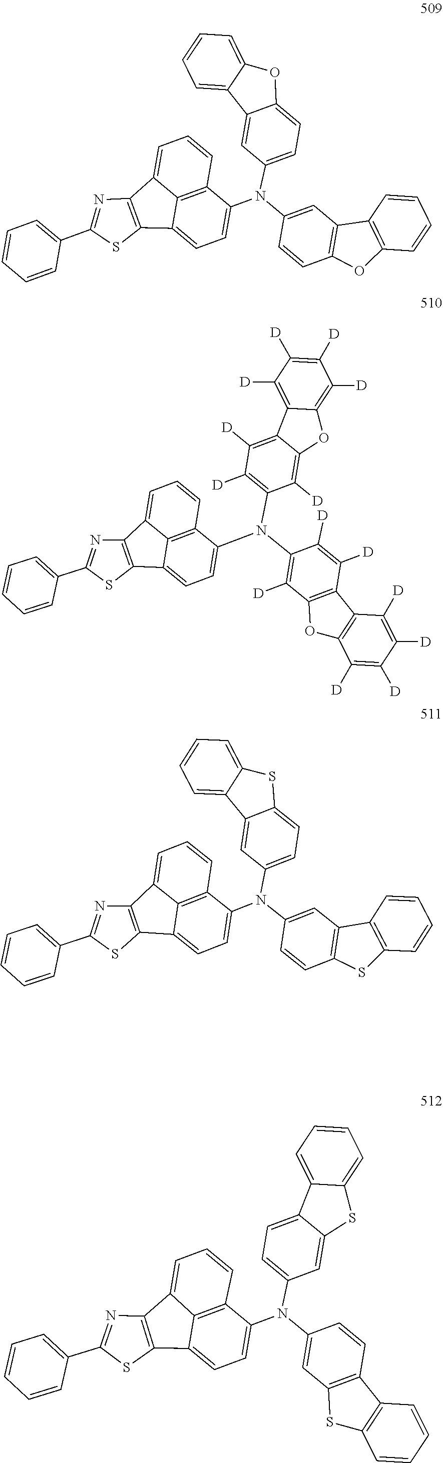 [SMILES: c1ccc(-c2nc3c(s2)-c2ccc(N(c4ccc5oc6ccccc6c5c4)c4ccc5oc6ccccc6c5c4)c4cccc-3c24)cc1 ; [2H]c1c([2H])c([2H])c2c(oc3c([2H])c(N(c4c([2H])c([2H])c5c(oc6c([2H])c([2H])c([2H])c([2H])c65)c4[2H])c4ccc5c6c(cccc46)-c4nc(-c6ccccc6)sc4-5)c([2H])c([2H])c32)c1[2H] ; c1ccc(-c2nc3c(s2)-c2ccc(N(c4ccc5sc6ccccc6c5c4)c4ccc5sc6ccccc6c5c4)c4cccc-3c24)cc1 ; c1ccc(-c2nc3c(s2)-c2ccc(N(c4ccc5c(c4)sc4ccccc45)c4ccc5c(c4)sc4ccccc45)c4cccc-3c24)cc1]

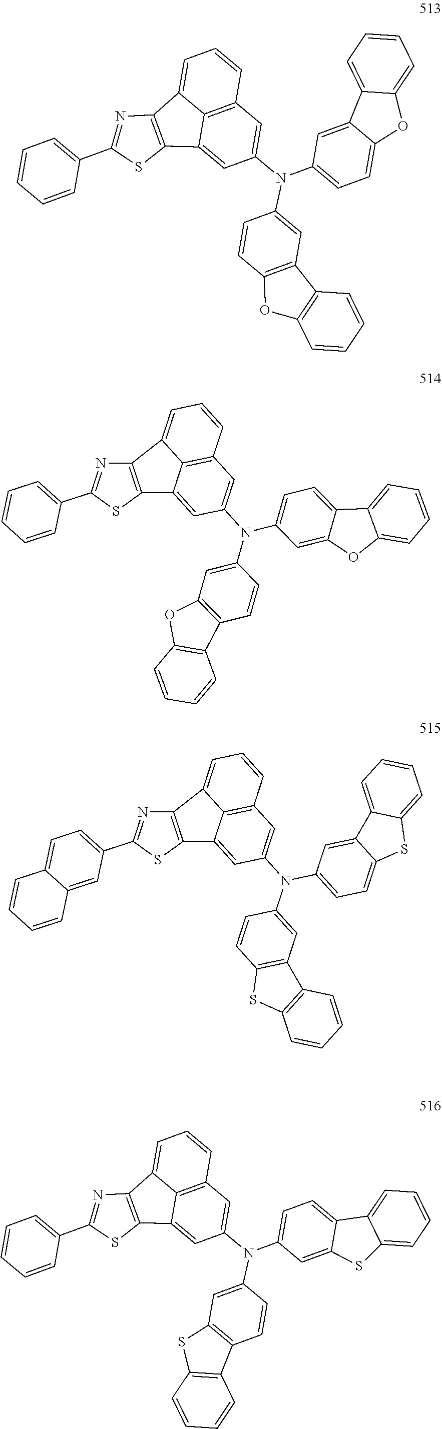 [SMILES: c1ccc(-c2nc3c(s2)-c2cc(N(c4ccc5oc6ccccc6c5c4)c4ccc5oc6ccccc6c5c4)cc4cccc-3c24)cc1 ; c1ccc(-c2nc3c(s2)-c2cc(N(c4ccc5c(c4)oc4ccccc45)c4ccc5c(c4)oc4ccccc45)cc4cccc-3c24)cc1 ; c1ccc2cc(-c3nc4c(s3)-c3cc(N(c5ccc6sc7ccccc7c6c5)c5ccc6sc7ccccc7c6c5)cc5cccc-4c35)ccc2c1 ; c1ccc(-c2nc3c(s2)-c2cc(N(c4ccc5c(c4)sc4ccccc45)c4ccc5c(c4)sc4ccccc45)cc4cccc-3c24)cc1]

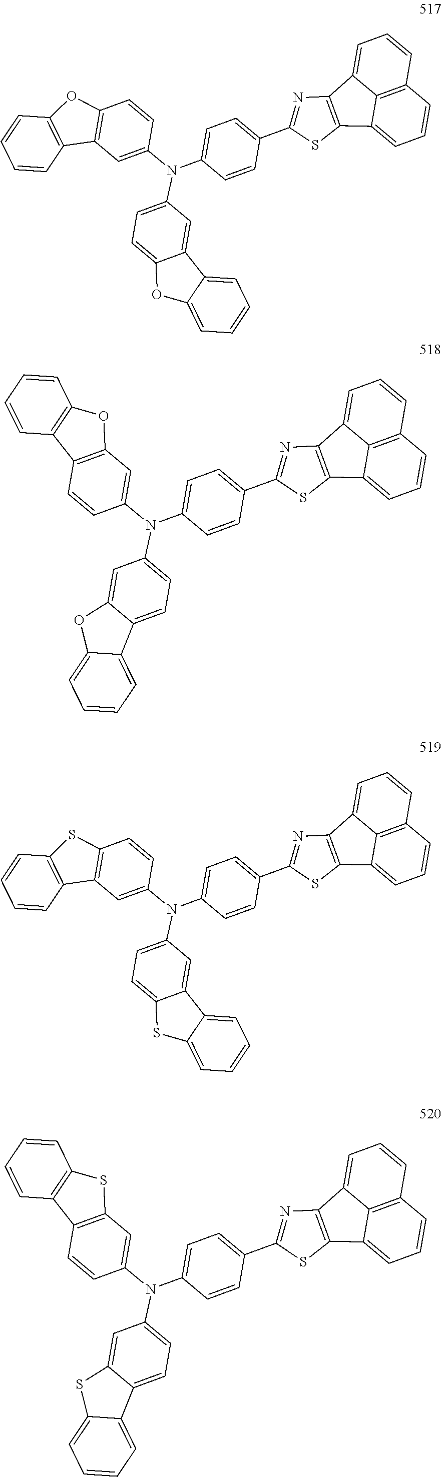 [SMILES: c1cc2c3c(cccc3c1)-c1sc(-c3ccc(N(c4ccc5oc6ccccc6c5c4)c4ccc5oc6ccccc6c5c4)cc3)nc1-2 ; c1cc2c3c(cccc3c1)-c1sc(-c3ccc(N(c4ccc5c(c4)oc4ccccc45)c4ccc5c(c4)oc4ccccc45)cc3)nc1-2 ; c1cc2c3c(cccc3c1)-c1sc(-c3ccc(N(c4ccc5sc6ccccc6c5c4)c4ccc5sc6ccccc6c5c4)cc3)nc1-2 ; c1cc2c3c(cccc3c1)-c1sc(-c3ccc(N(c4ccc5c(c4)sc4ccccc45)c4ccc5c(c4)sc4ccccc45)cc3)nc1-2]

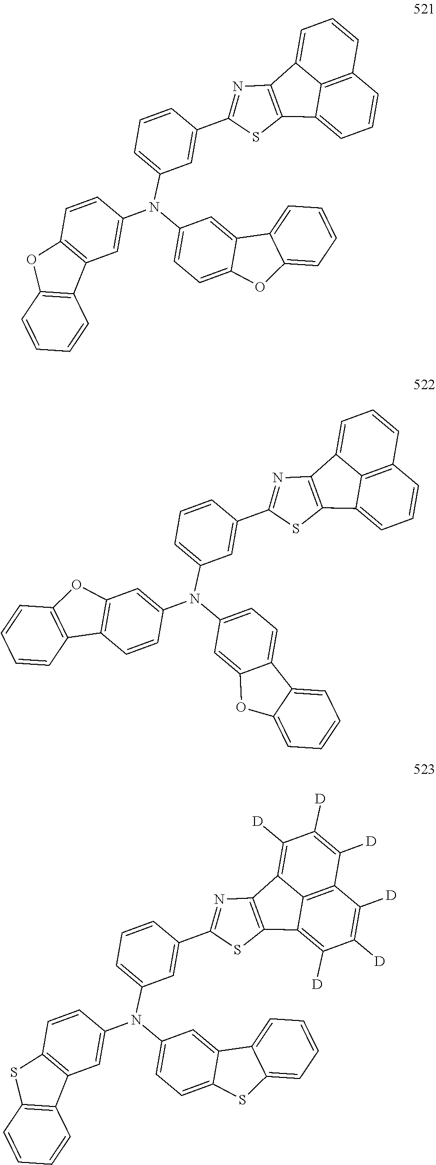 [SMILES: c1cc(-c2nc3c(s2)-c2cccc4cccc-3c24)cc(N(c2ccc3oc4ccccc4c3c2)c2ccc3oc4ccccc4c3c2)c1 ; c1cc(-c2nc3c(s2)-c2cccc4cccc-3c24)cc(N(c2ccc3c(c2)oc2ccccc23)c2ccc3c(c2)oc2ccccc23)c1 ; [2H]c1c([2H])c2c3c(c([2H])c([2H])c([2H])c3c1[2H])-c1sc(-c3cccc(N(c4ccc5sc6ccccc6c5c4)c4ccc5sc6ccccc6c5c4)c3)nc1-2]

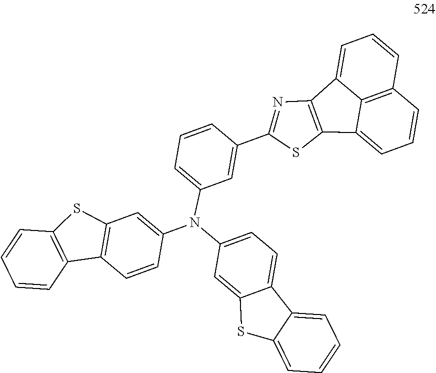 [SMILES: c1cc(-c2nc3c(s2)-c2cccc4cccc-3c24)cc(N(c2ccc3c(c2)sc2ccccc23)c2ccc3c(c2)sc2ccccc23)c1]

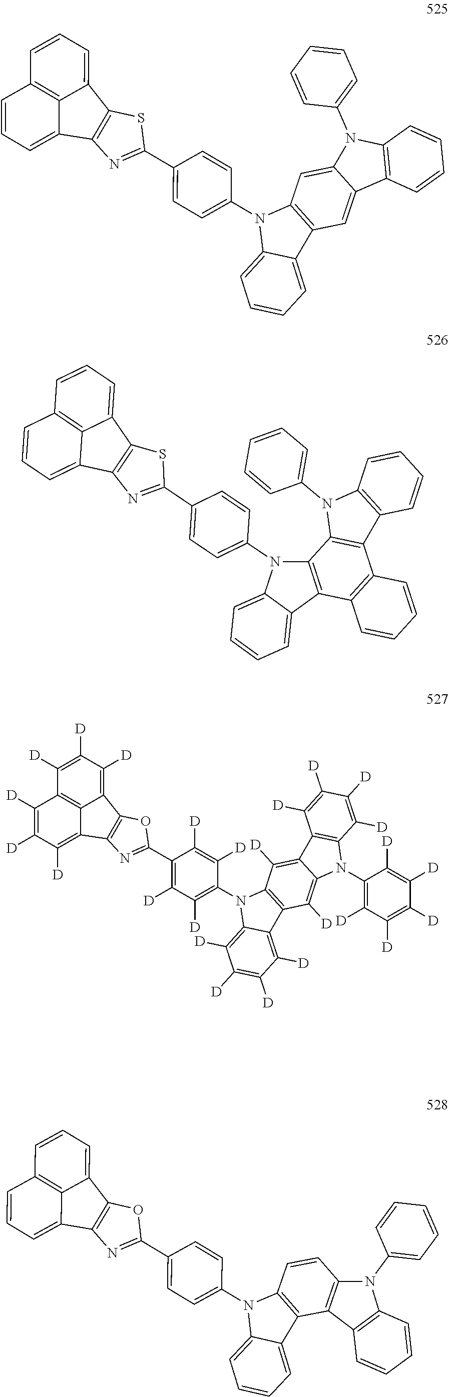 [SMILES: c1ccc(-n2c3ccccc3c3cc4c5ccccc5n(-c5ccc(-c6nc7c(s6)-c6cccc8cccc-7c68)cc5)c4cc32)cc1 ; c1ccc(-n2c3ccccc3c3c4ccccc4c4c5ccccc5n(-c5ccc(-c6nc7c(s6)-c6cccc8cccc-7c68)cc5)c4c32)cc1 ; [2H]c1c([2H])c([2H])c(-n2c3c([2H])c([2H])c([2H])c([2H])c3c3c([2H])c4c(c([2H])c32)c2c([2H])c([2H])c([2H])c([2H])c2n4-c2c([2H])c([2H])c(-c3nc4c(o3)-c3c([2H])c([2H])c([2H])c5c([2H])c([2H])c([2H])c-4c35)c([2H])c2[2H])c([2H])c1[2H] ; c1ccc(-n2c3ccccc3c3c4c5ccccc5n(-c5ccc(-c6nc7c(o6)-c6cccc8cccc-7c68)cc5)c4ccc32)cc1]

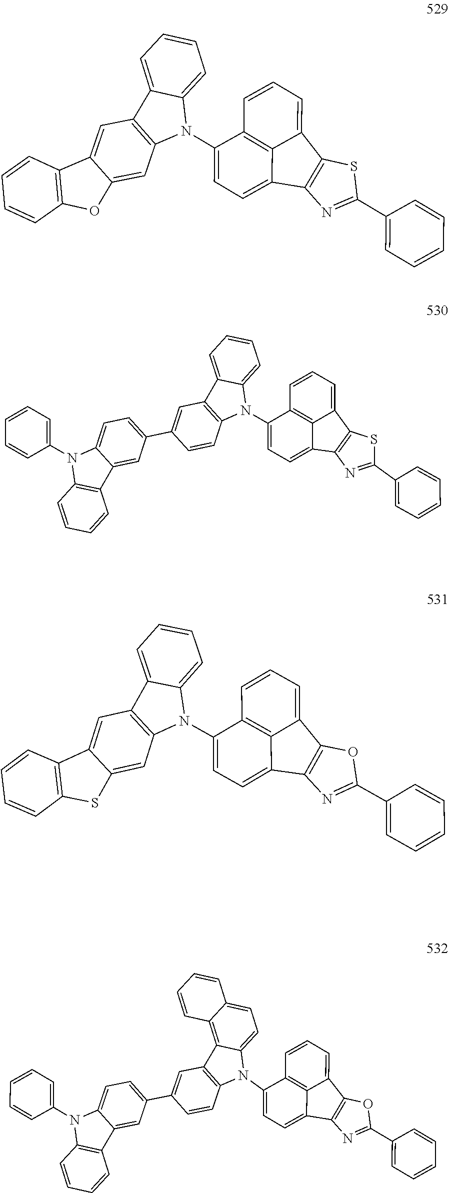

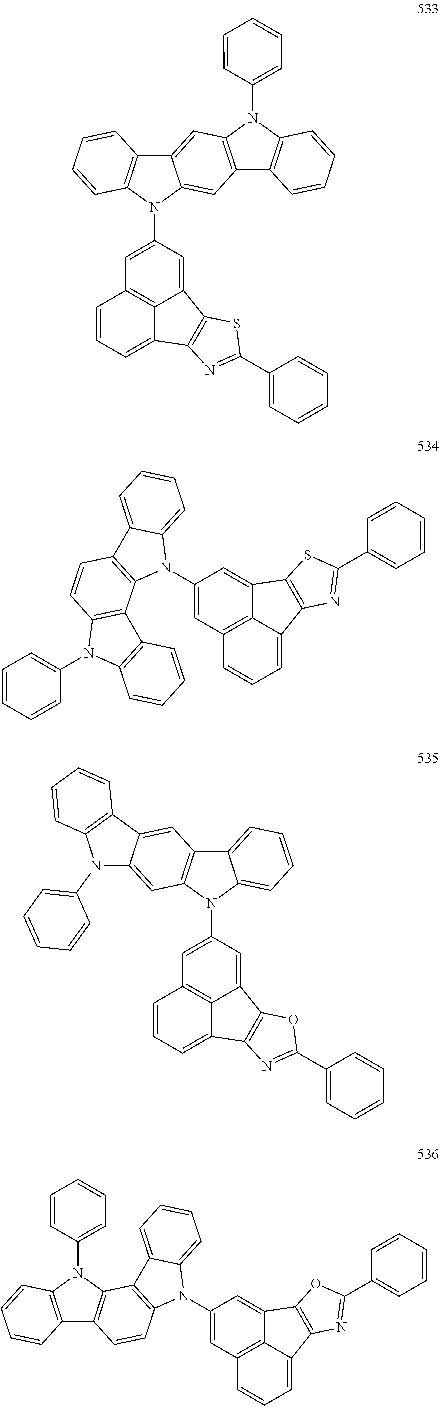 [SMILES: c1ccc(-c2nc3c(s2)-c2cc(-n4c5ccccc5c5cc6c(cc54)c4ccccc4n6-c4ccccc4)cc4cccc-3c24)cc1 ; c1ccc(-c2nc3c(s2)-c2cc(-n4c5ccccc5c5ccc6c(c7ccccc7n6-c6ccccc6)c54)cc4cccc-3c24)cc1 ; c1ccc(-c2nc3c(o2)-c2cc(-n4c5ccccc5c5cc6c7ccccc7n(-c7ccccc7)c6cc54)cc4cccc-3c24)cc1 ; c1ccc(-c2nc3c(o2)-c2cc(-n4c5ccccc5c5c4ccc4c6ccccc6n(-c6ccccc6)c45)cc4cccc-3c24)cc1]

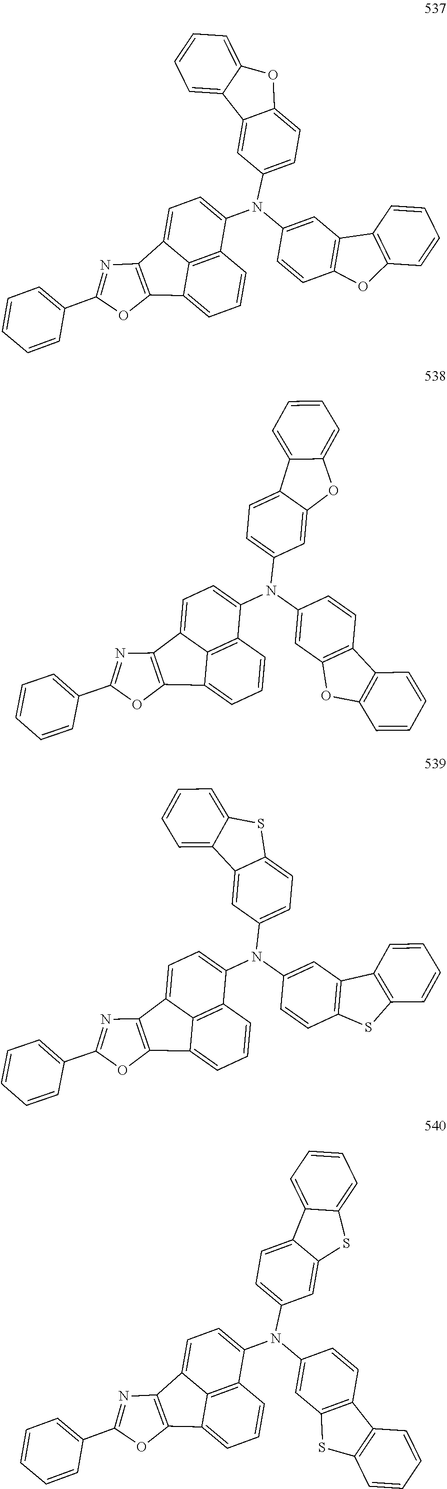 [SMILES: c1ccc(-c2nc3c(o2)-c2cccc4c(N(c5ccc6oc7ccccc7c6c5)c5ccc6oc7ccccc7c6c5)ccc-3c24)cc1 ; c1ccc(-c2nc3c(o2)-c2cccc4c(N(c5ccc6c(c5)oc5ccccc56)c5ccc6c(c5)oc5ccccc56)ccc-3c24)cc1 ; c1ccc(-c2nc3c(o2)-c2cccc4c(N(c5ccc6sc7ccccc7c6c5)c5ccc6sc7ccccc7c6c5)ccc-3c24)cc1 ; c1ccc(-c2nc3c(o2)-c2cccc4c(N(c5ccc6c(c5)sc5ccccc56)c5ccc6c(c5)sc5ccccc56)ccc-3c24)cc1]

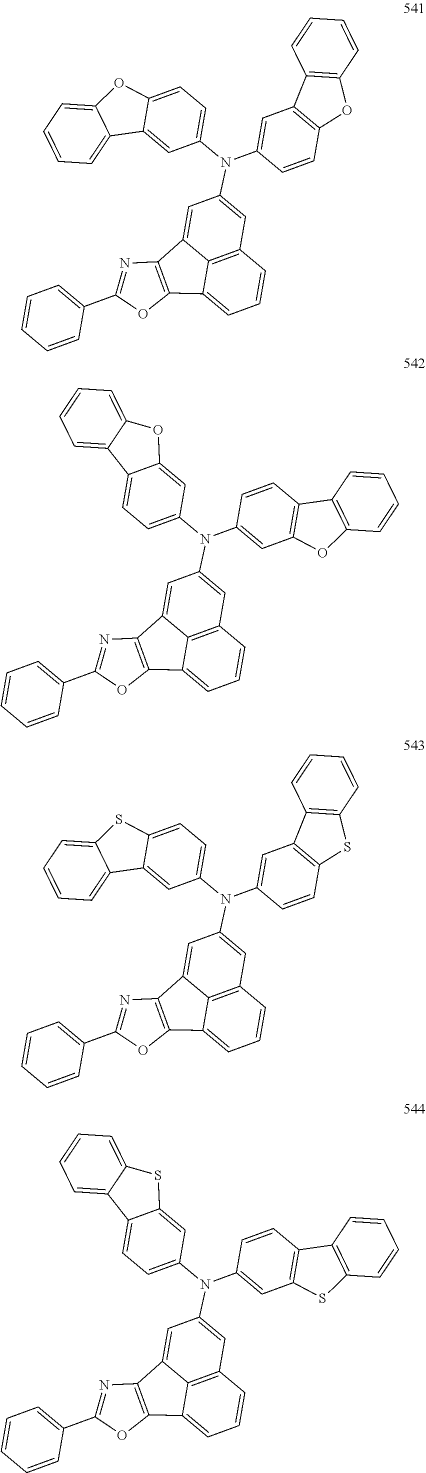 [SMILES: c1ccc(-c2nc3c(o2)-c2cccc4cc(N(c5ccc6oc7ccccc7c6c5)c5ccc6oc7ccccc7c6c5)cc-3c24)cc1 ; c1ccc(-c2nc3c(o2)-c2cccc4cc(N(c5ccc6c(c5)oc5ccccc56)c5ccc6c(c5)oc5ccccc56)cc-3c24)cc1 ; c1ccc(-c2nc3c(o2)-c2cccc4cc(N(c5ccc6sc7ccccc7c6c5)c5ccc6sc7ccccc7c6c5)cc-3c24)cc1 ; c1ccc(-c2nc3c(o2)-c2cccc4cc(N(c5ccc6c(c5)sc5ccccc56)c5ccc6c(c5)sc5ccccc56)cc-3c24)cc1]

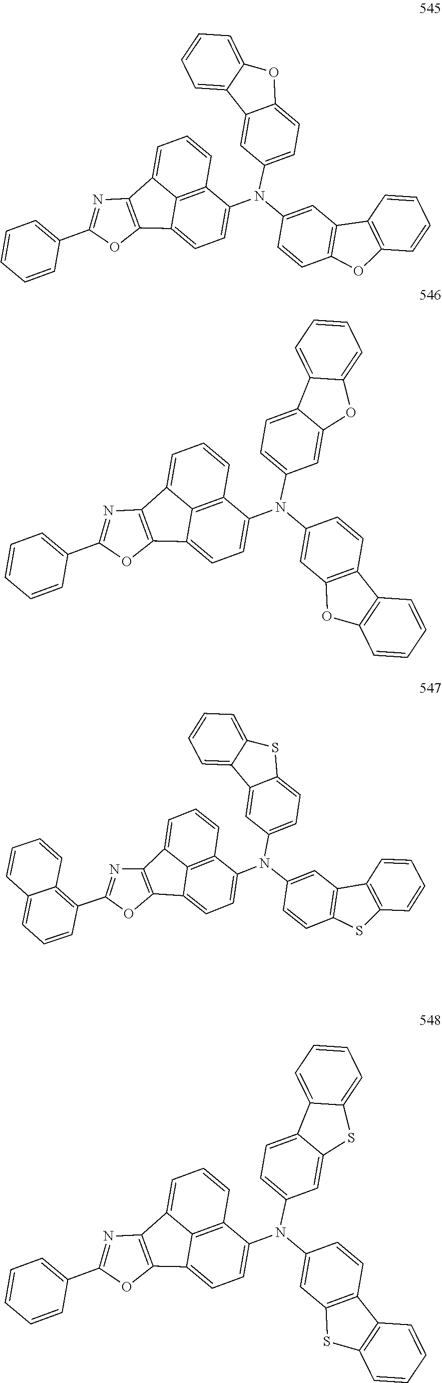 [SMILES: c1ccc(-c2nc3c(o2)-c2ccc(N(c4ccc5oc6ccccc6c5c4)c4ccc5oc6ccccc6c5c4)c4cccc-3c24)cc1 ; c1ccc(-c2nc3c(o2)-c2ccc(N(c4ccc5c(c4)oc4ccccc45)c4ccc5c(c4)oc4ccccc45)c4cccc-3c24)cc1 ; c1ccc2c(-c3nc4c(o3)-c3ccc(N(c5ccc6sc7ccccc7c6c5)c5ccc6sc7ccccc7c6c5)c5cccc-4c35)cccc2c1 ; c1ccc(-c2nc3c(o2)-c2ccc(N(c4ccc5c(c4)sc4ccccc45)c4ccc5c(c4)sc4ccccc45)c4cccc-3c24)cc1]

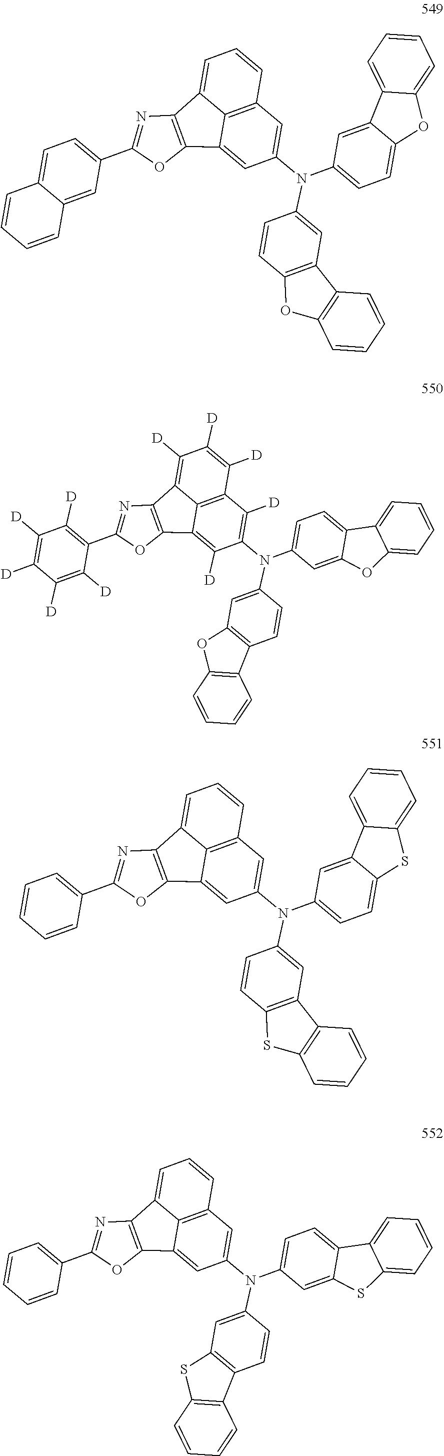 [SMILES: c1ccc2cc(-c3nc4c(o3)-c3cc(N(c5ccc6oc7ccccc7c6c5)c5ccc6oc7ccccc7c6c5)cc5cccc-4c35)ccc2c1 ; [2H]c1c([2H])c([2H])c(-c2nc3c(o2)-c2c([2H])c(N(c4ccc5c(c4)oc4ccccc45)c4ccc5c(c4)oc4ccccc45)c([2H])c4c([2H])c([2H])c([2H])c-3c24)c([2H])c1[2H] ; c1ccc(-c2nc3c(o2)-c2cc(N(c4ccc5sc6ccccc6c5c4)c4ccc5sc6ccccc6c5c4)cc4cccc-3c24)cc1 ; c1ccc(-c2nc3c(o2)-c2cc(N(c4ccc5c(c4)sc4ccccc45)c4ccc5c(c4)sc4ccccc45)cc4cccc-3c24)cc1]

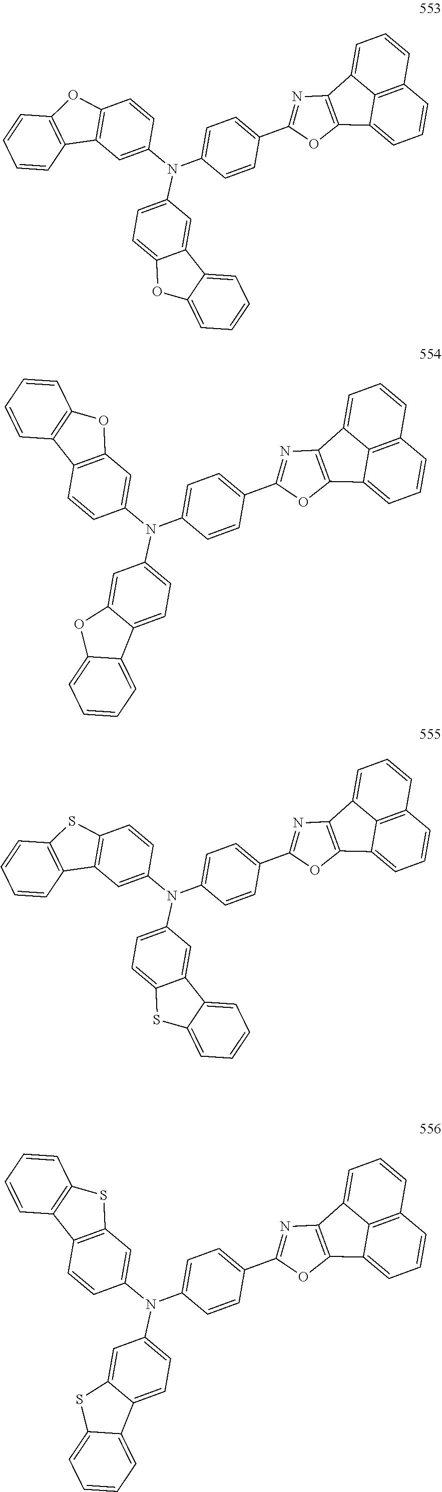 [SMILES: c1cc2c3c(cccc3c1)-c1oc(-c3ccc(N(c4ccc5oc6ccccc6c5c4)c4ccc5oc6ccccc6c5c4)cc3)nc1-2 ; c1cc2c3c(cccc3c1)-c1oc(-c3ccc(N(c4ccc5c(c4)oc4ccccc45)c4ccc5c(c4)oc4ccccc45)cc3)nc1-2 ; c1cc2c3c(cccc3c1)-c1oc(-c3ccc(N(c4ccc5sc6ccccc6c5c4)c4ccc5sc6ccccc6c5c4)cc3)nc1-2 ; c1cc2c3c(cccc3c1)-c1oc(-c3ccc(N(c4ccc5c(c4)sc4ccccc45)c4ccc5c(c4)sc4ccccc45)cc3)nc1-2]

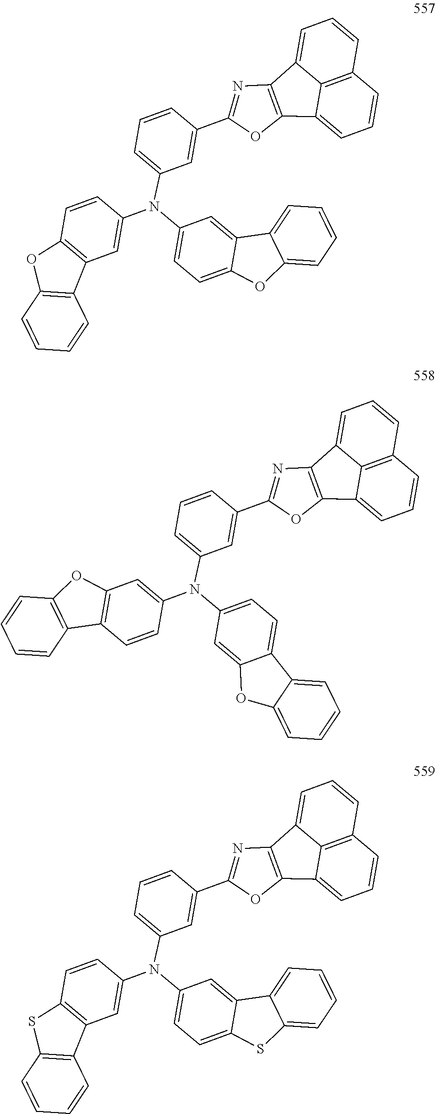

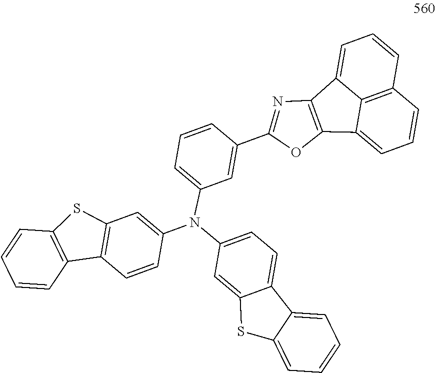 [SMILES: c1cc(-c2nc3c(o2)-c2cccc4cccc-3c24)cc(N(c2ccc3c(c2)sc2ccccc23)c2ccc3c(c2)sc2ccccc23)c1]

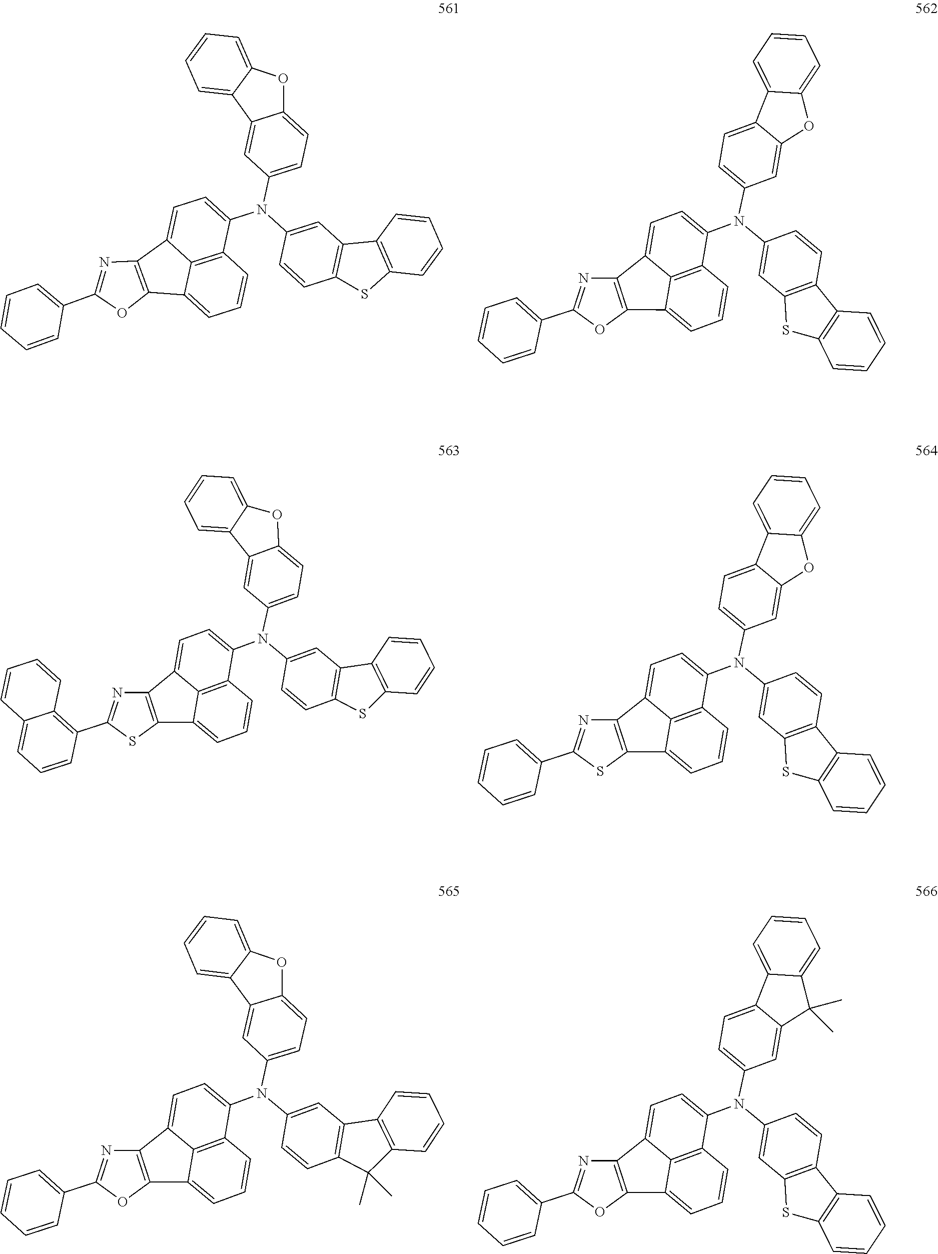

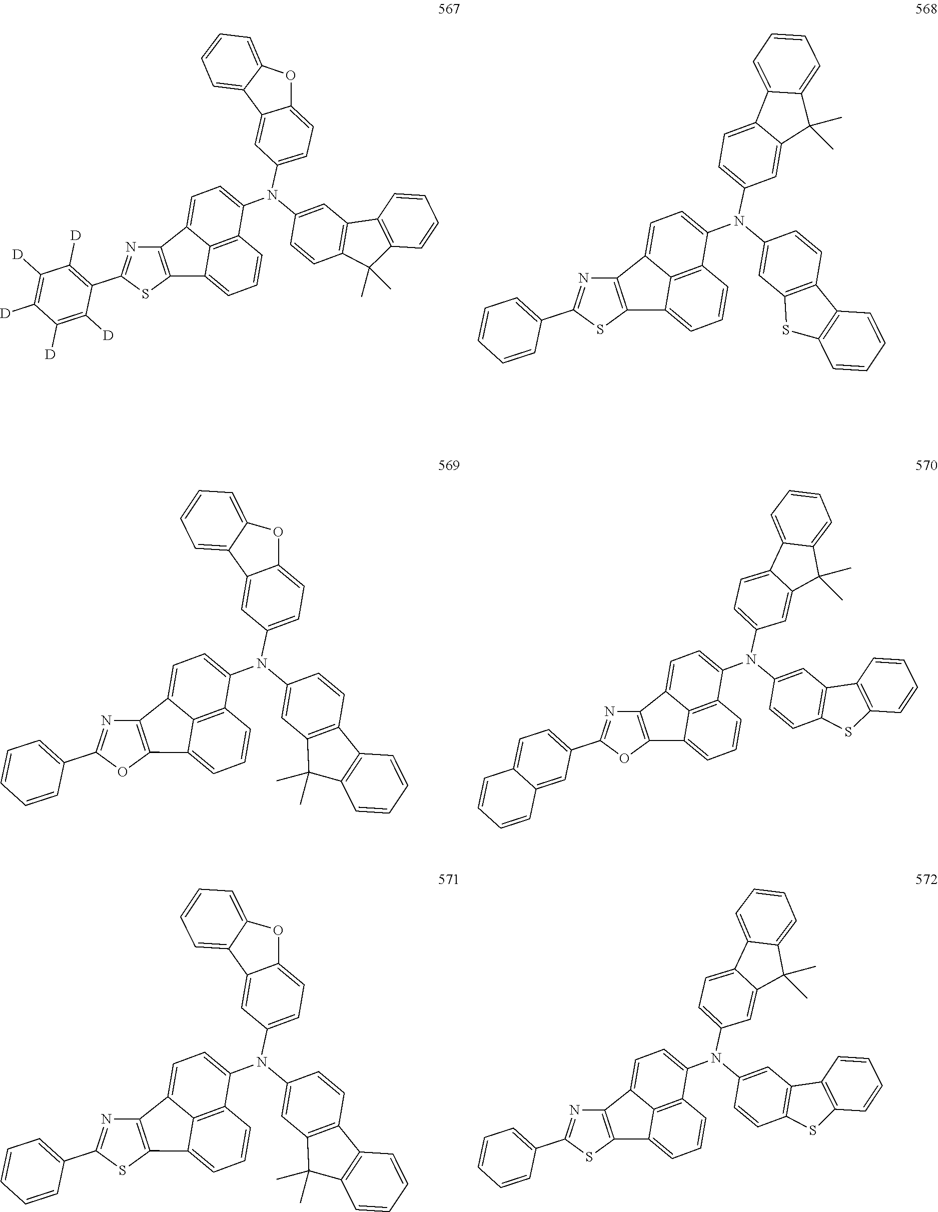

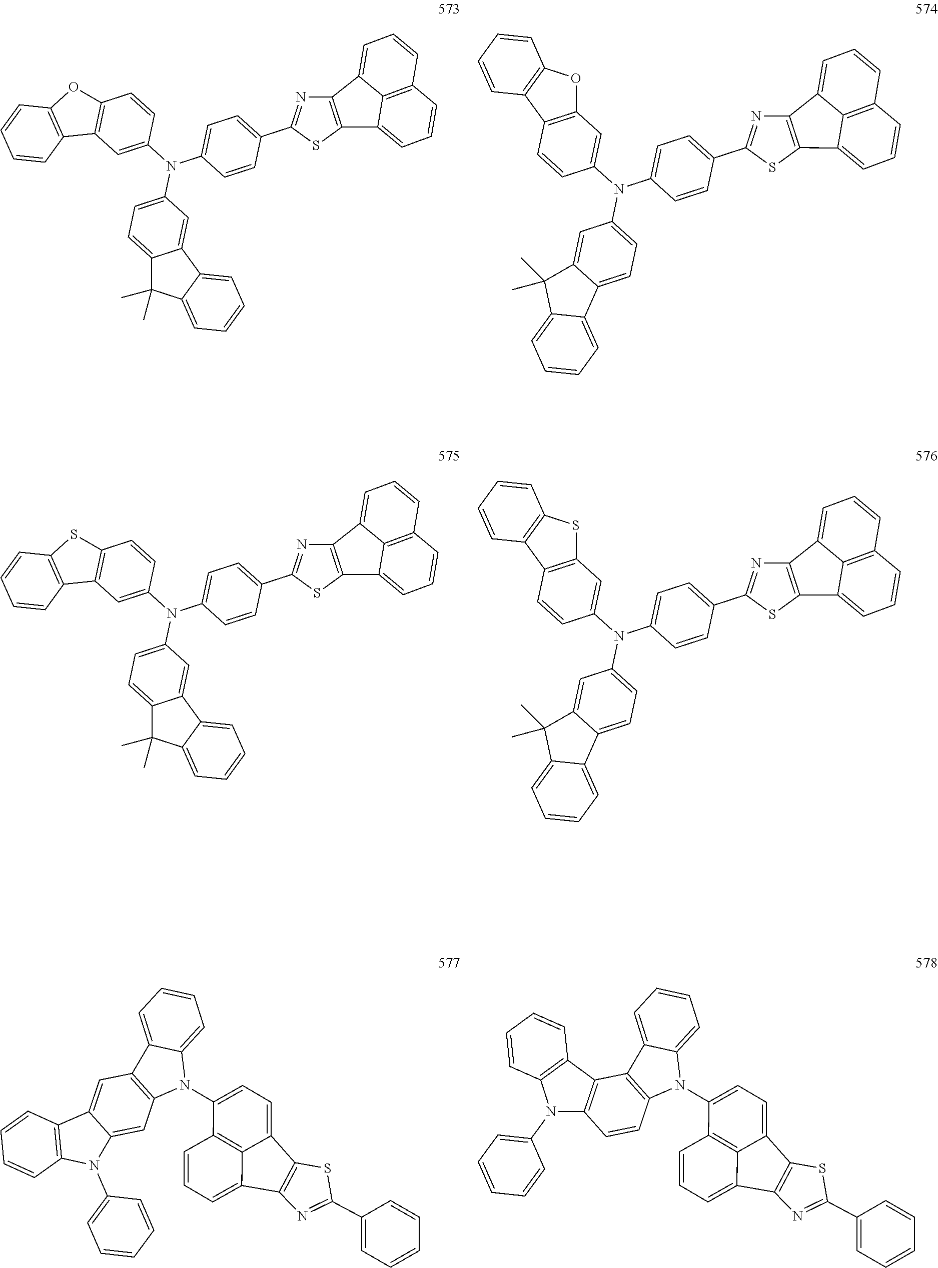

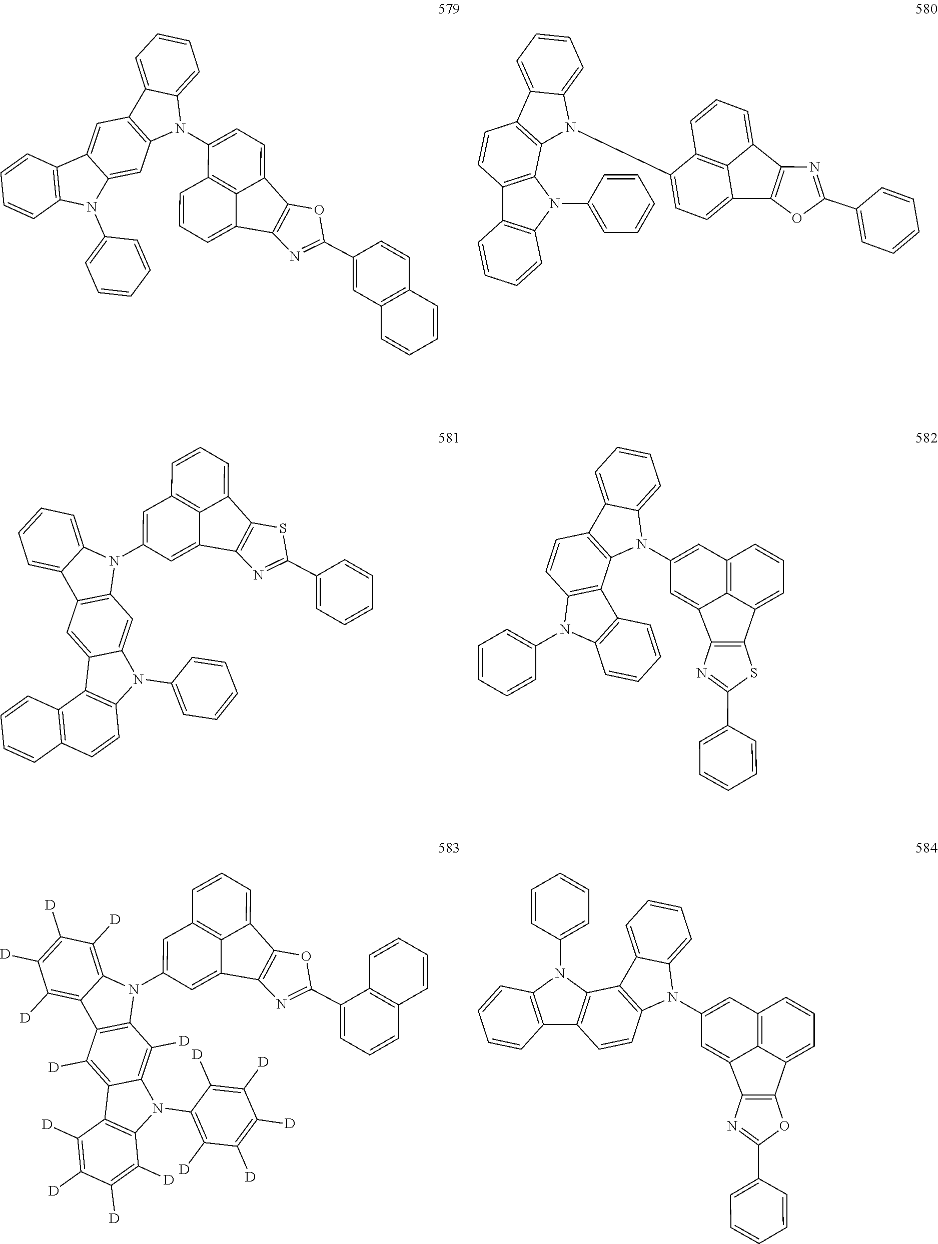

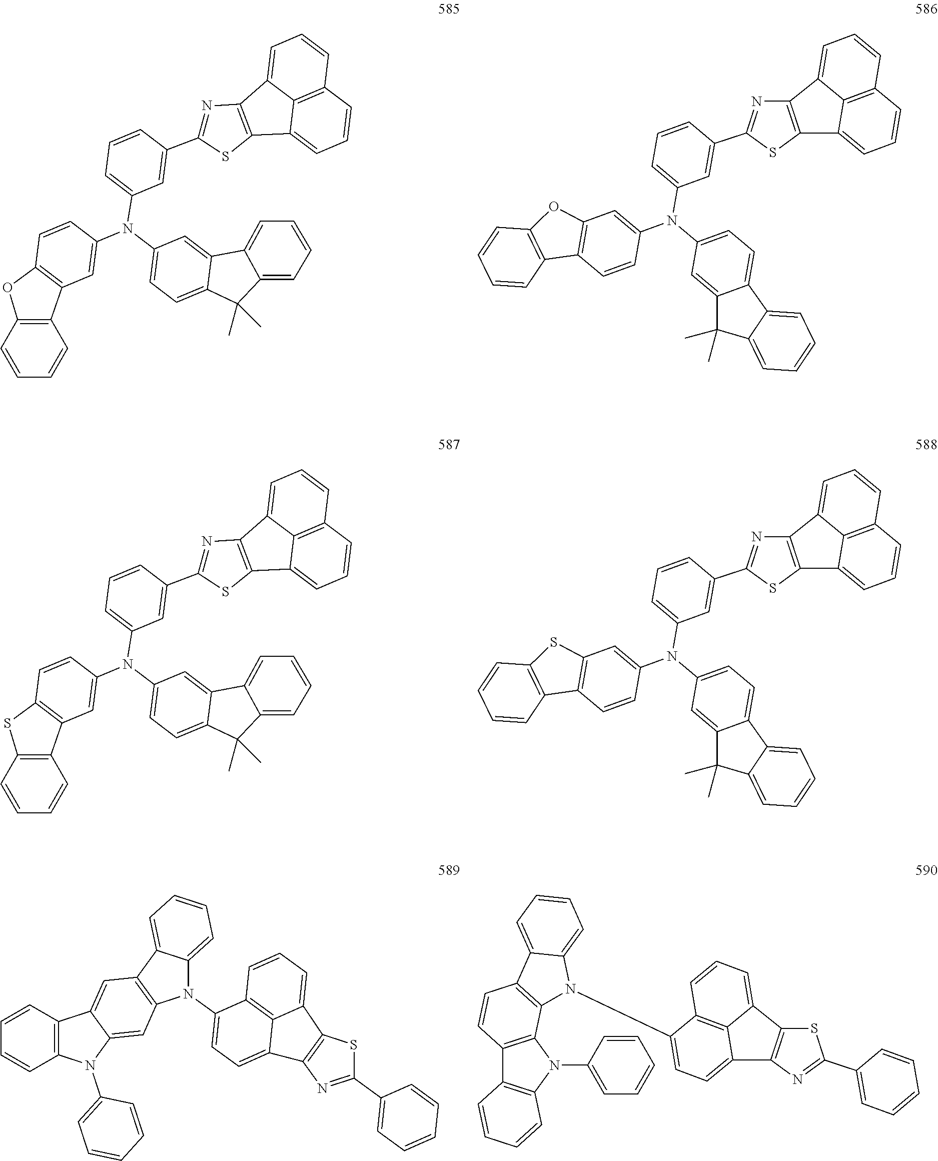

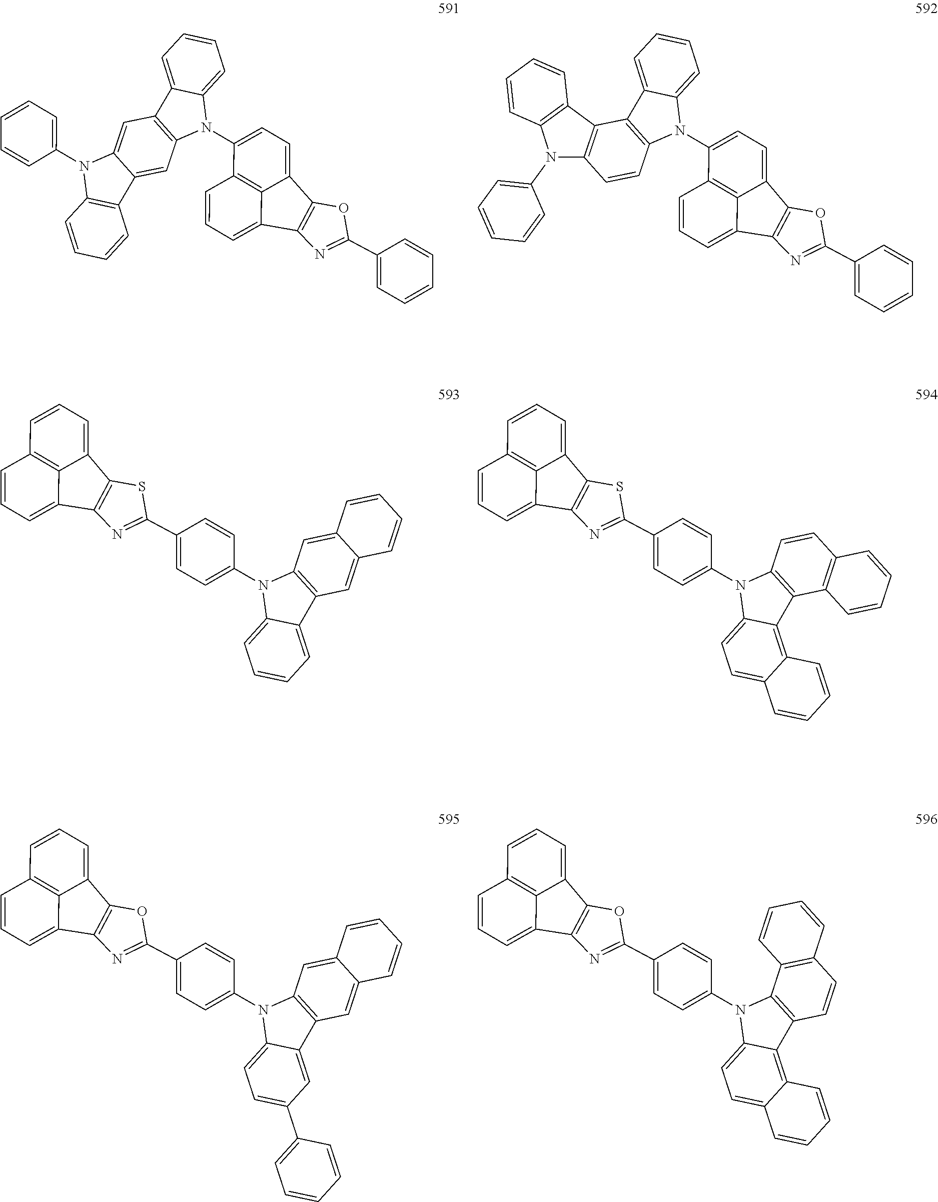 [SMILES: c1ccc(-c2nc3c(o2)-c2ccc(-n4c5ccccc5c5cc6c(cc54)c4ccccc4n6-c4ccccc4)c4cccc-3c24)cc1 ; c1ccc(-c2nc3c(o2)-c2ccc(-n4c5ccccc5c5c6c7ccccc7n(-c7ccccc7)c6ccc54)c4cccc-3c24)cc1 ; c1ccc2cc3c(cc2c1)c1ccccc1n3-c1ccc(-c2nc3c(s2)-c2cccc4cccc-3c24)cc1 ; c1cc2c3c(cccc3c1)-c1sc(-c3ccc(-n4c5ccc6ccccc6c5c5c6ccccc6ccc54)cc3)nc1-2 ; c1ccc(-c2ccc3c(c2)c2cc4ccccc4cc2n3-c2ccc(-c3nc4c(o3)-c3cccc5cccc-4c35)cc2)cc1 ; c1cc2c3c(cccc3c1)-c1oc(-c3ccc(-n4c5ccc6ccccc6c5c5ccc6ccccc6c54)cc3)nc1-2]

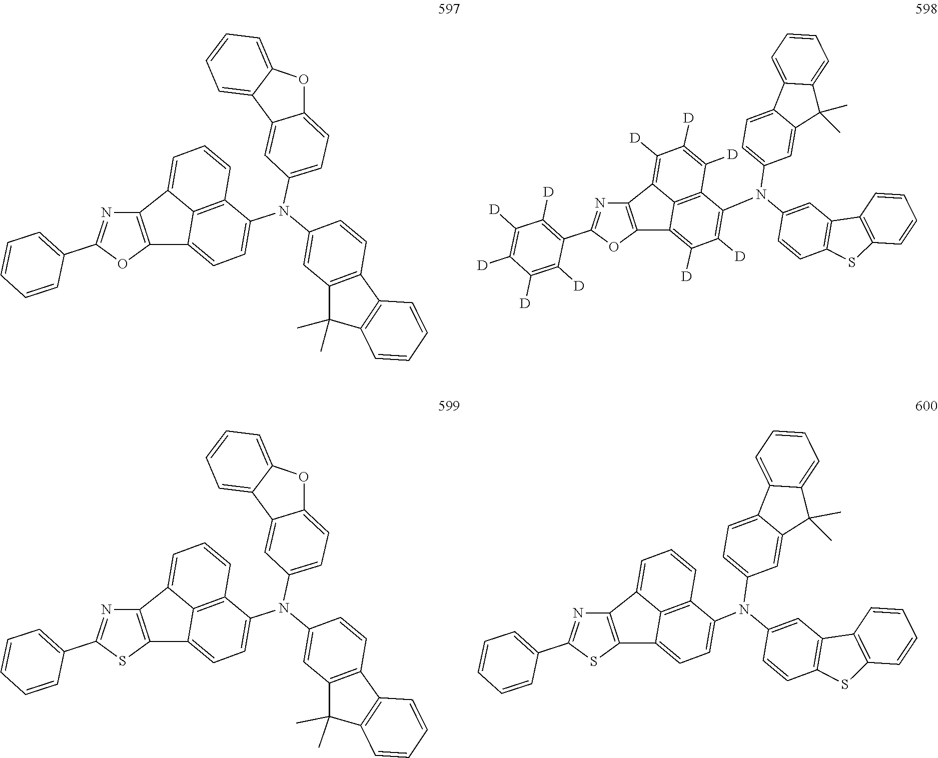 [SMILES: CC1(C)c2ccccc2-c2ccc(N(c3ccc4oc5ccccc5c4c3)c3ccc4c5c(cccc35)-c3nc(-c5ccccc5)oc3-4)cc21 ; [2H]c1c([2H])c([2H])c(-c2nc3c(o2)-c2c([2H])c([2H])c(N(c4ccc5c(c4)C(C)(C)c4ccccc4-5)c4ccc5sc6ccccc6c5c4)c4c([2H])c([2H])c([2H])c-3c24)c([2H])c1[2H] ; CC1(C)c2ccccc2-c2ccc(N(c3ccc4oc5ccccc5c4c3)c3ccc4c5c(cccc35)-c3nc(-c5ccccc5)sc3-4)cc21 ; CC1(C)c2ccccc2-c2ccc(N(c3ccc4sc5ccccc5c4c3)c3ccc4c5c(cccc35)-c3nc(-c5ccccc5)sc3-4)cc21]

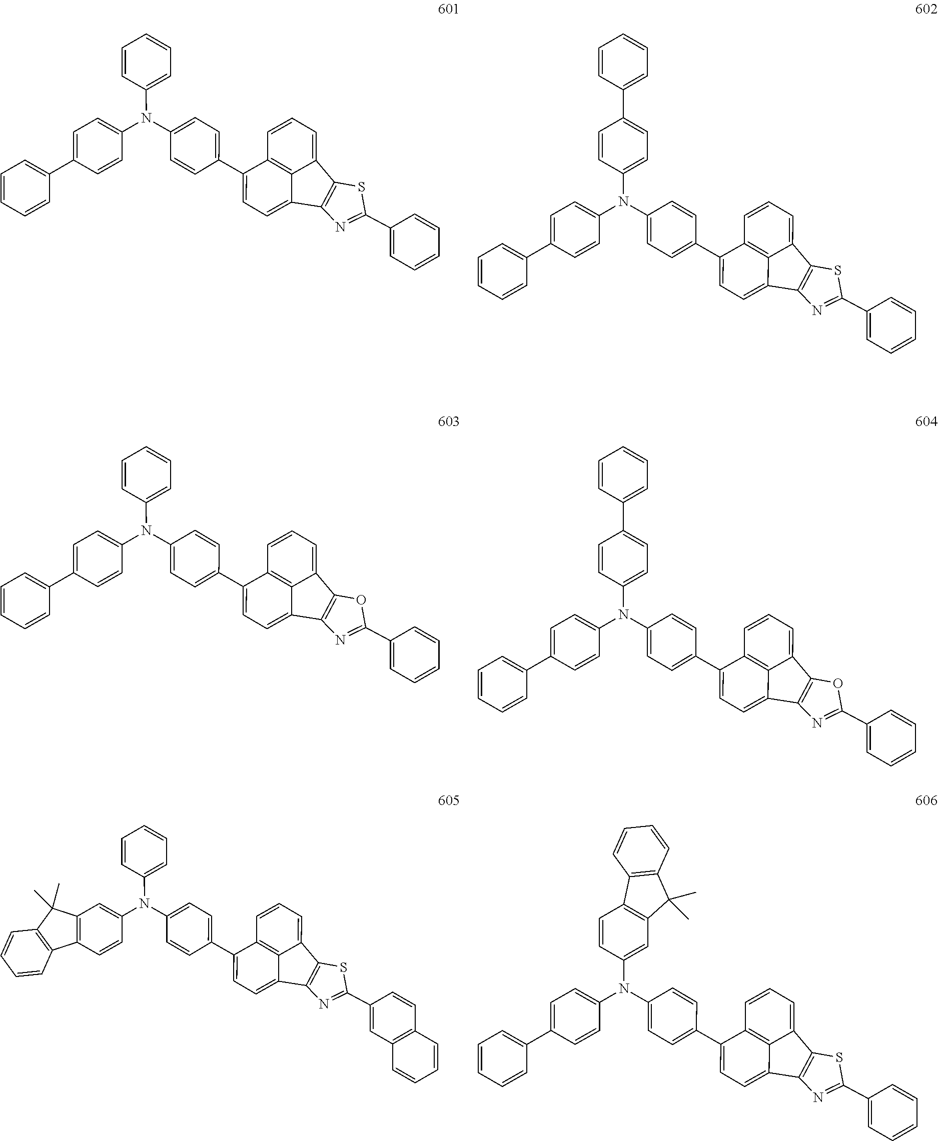

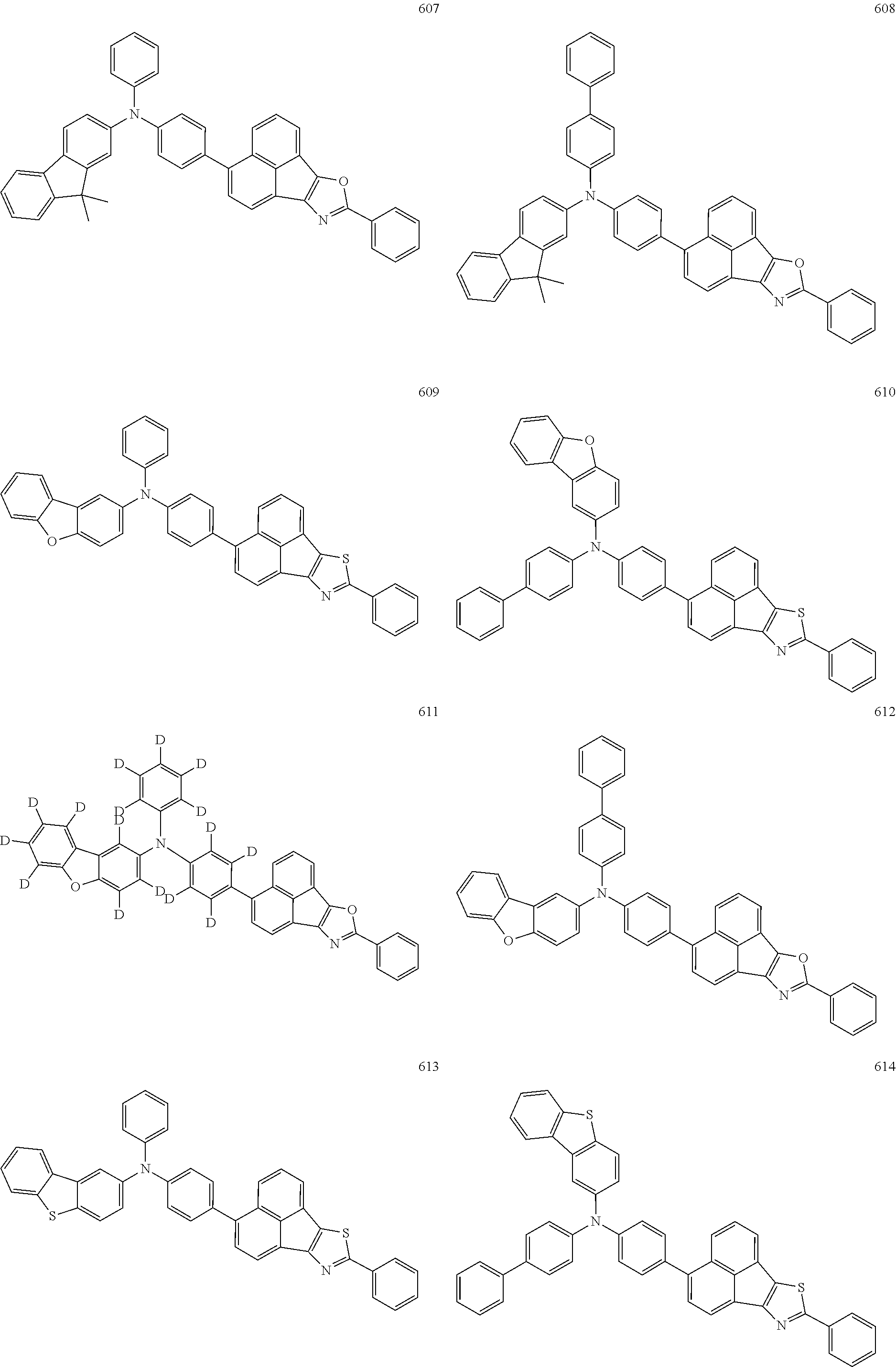

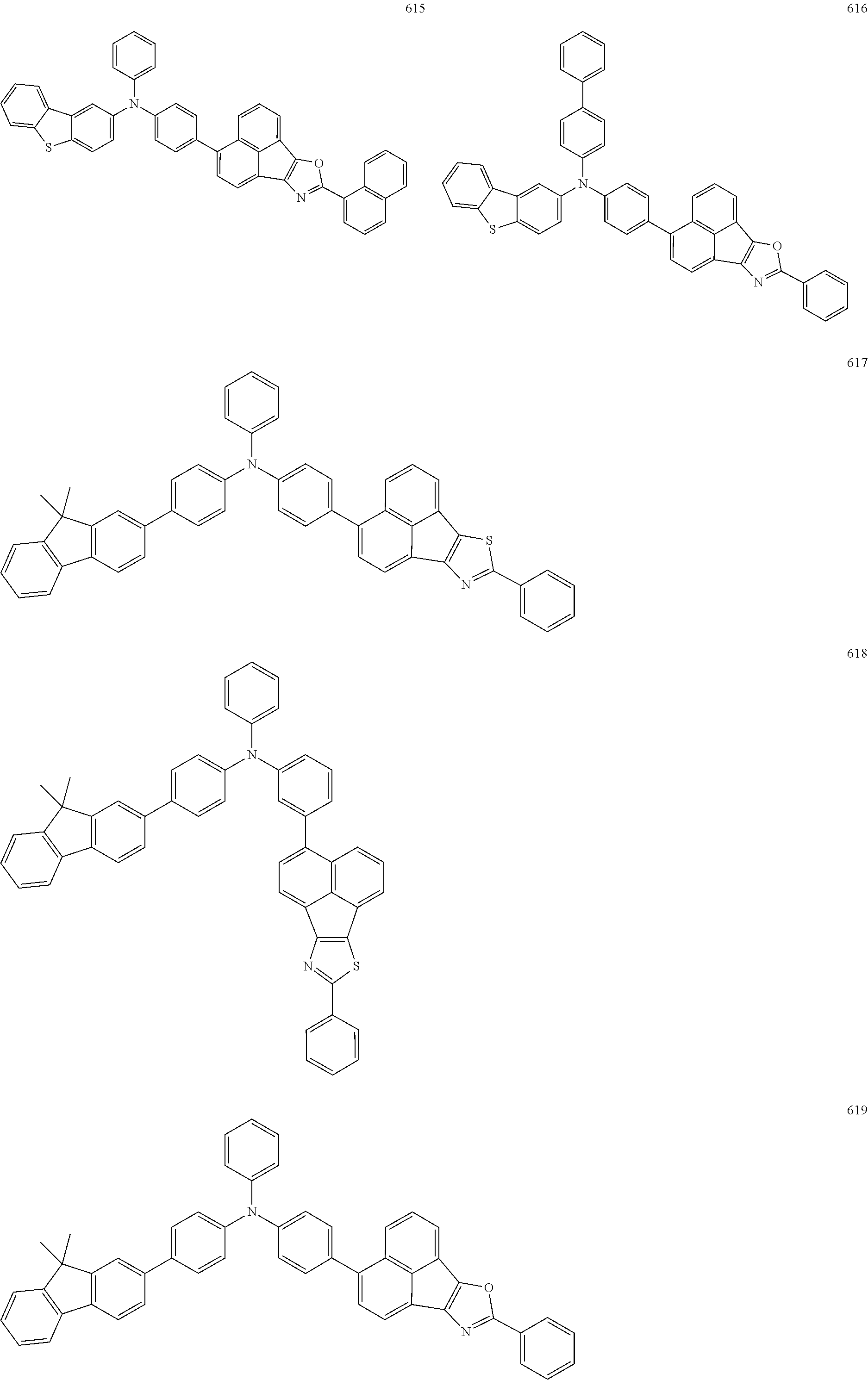

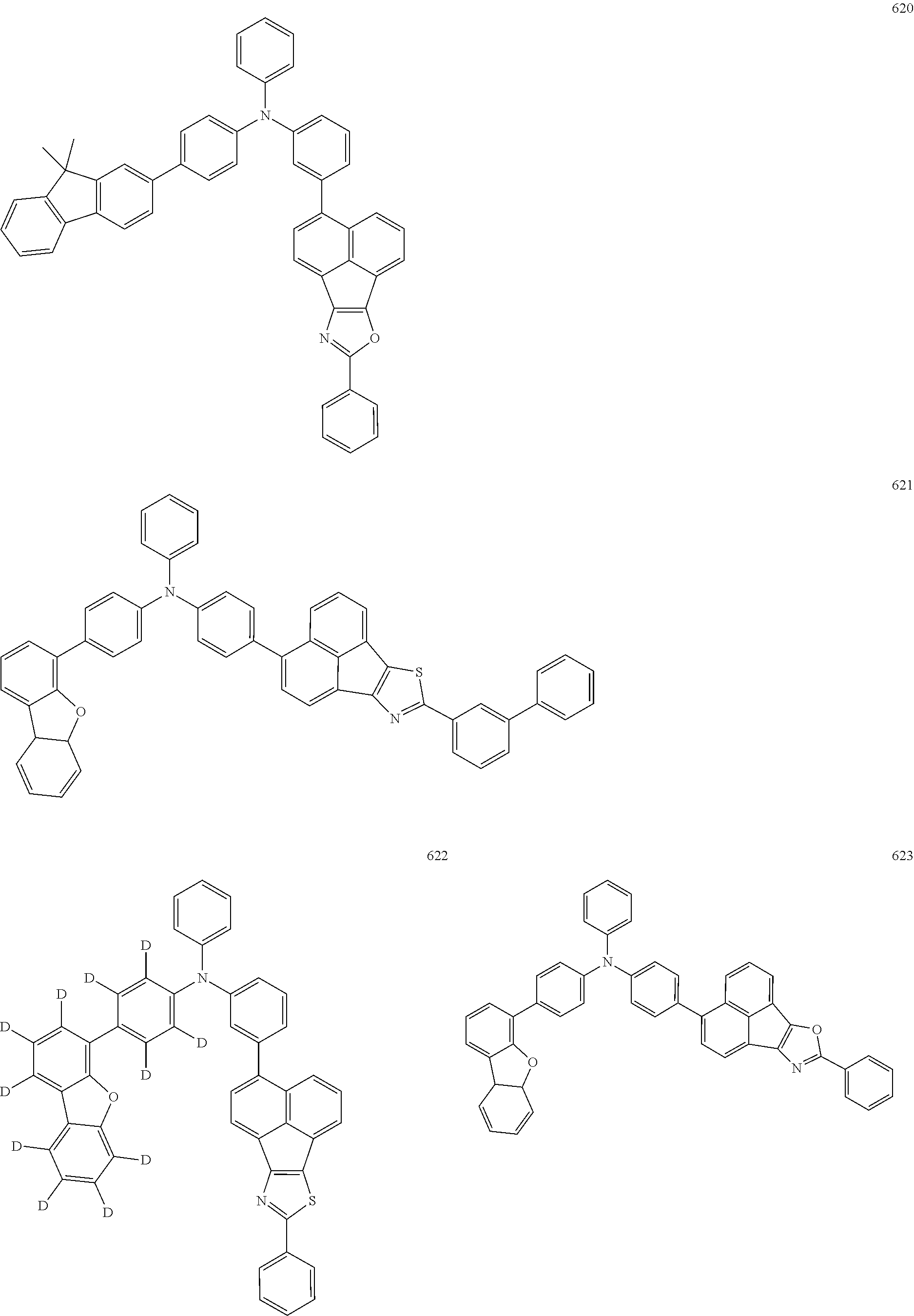 [SMILES: CC1(C)c2ccccc2-c2ccc(-c3ccc(N(c4ccccc4)c4cccc(-c5ccc6c7c(cccc57)-c5oc(-c7ccccc7)nc5-6)c4)cc3)cc21 ; C1=CC2Oc3c(-c4ccc(N(c5ccccc5)c5ccc(-c6ccc7c8c(cccc68)-c6sc(-c8cccc(-c9ccccc9)c8)nc6-7)cc5)cc4)cccc3C2C=C1 ; [2H]c1c([2H])c(N(c2ccccc2)c2cccc(-c3ccc4c5c(cccc35)-c3sc(-c5ccccc5)nc3-4)c2)c([2H])c([2H])c1-c1c([2H])c([2H])c([2H])c2c1oc1c([2H])c([2H])c([2H])c([2H])c12 ; C1=CC2Oc3c(-c4ccc(N(c5ccccc5)c5ccc(-c6ccc7c8c(cccc68)-c6oc(-c8ccccc8)nc6-7)cc5)cc4)cccc3C2C=C1]

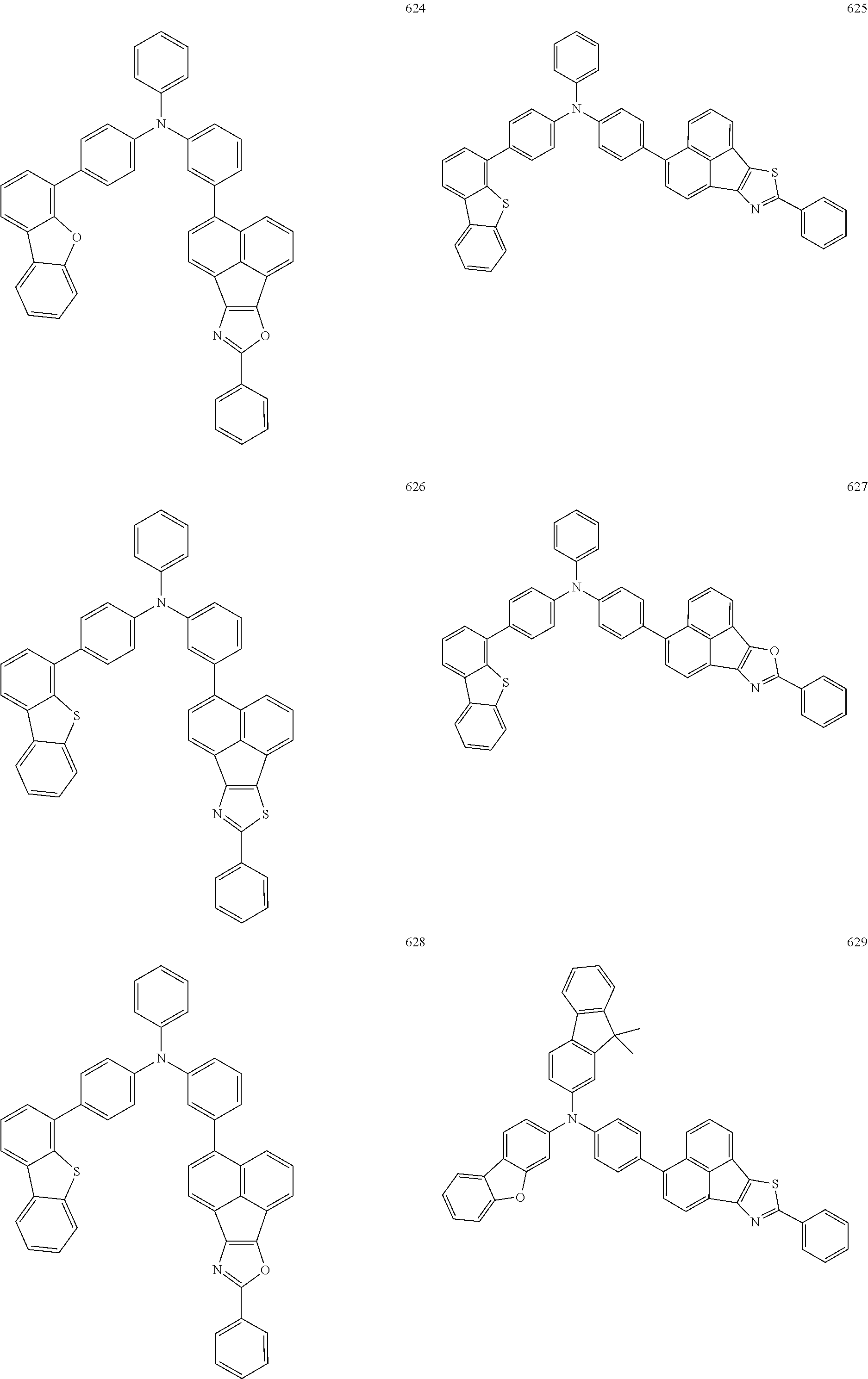

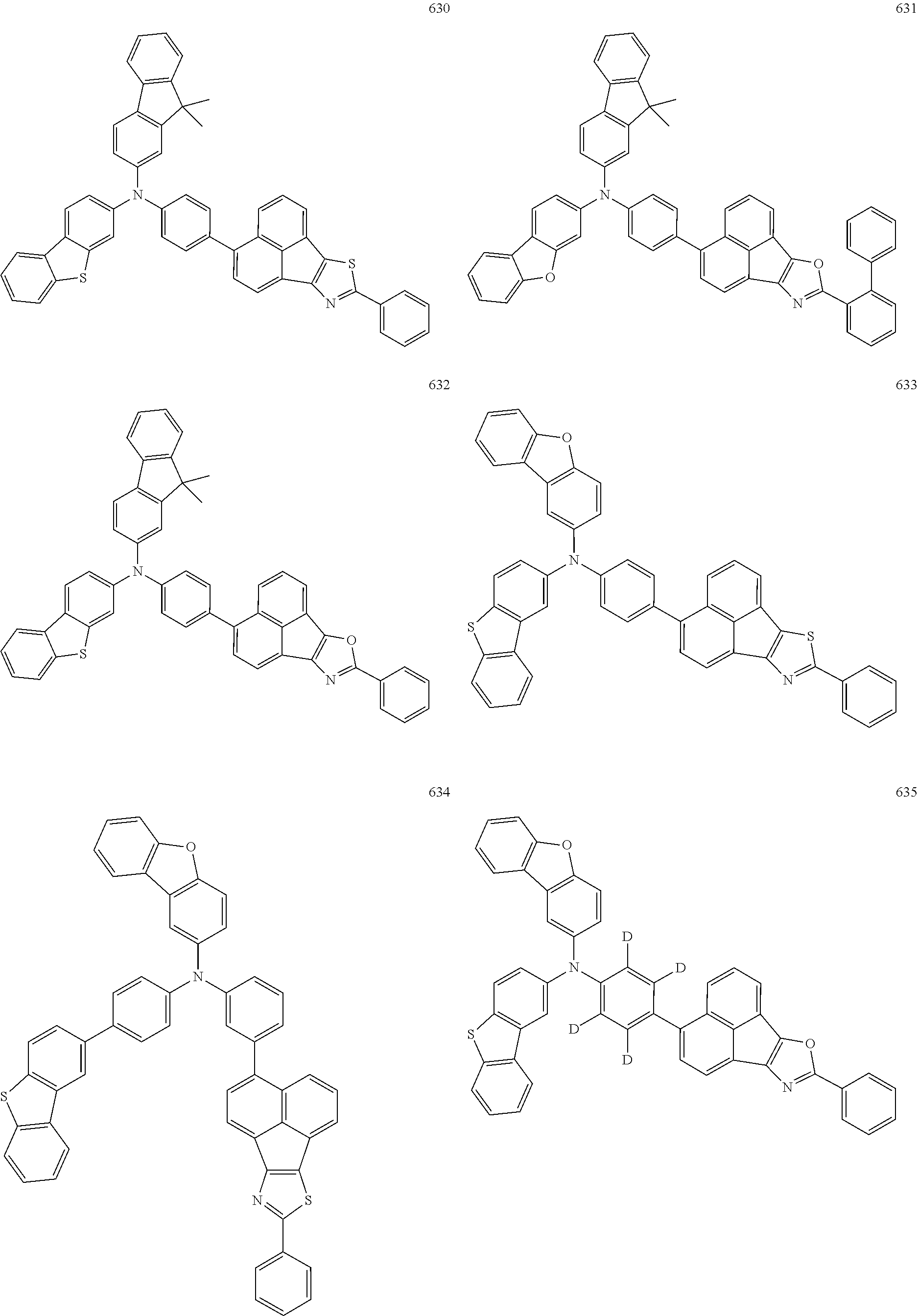

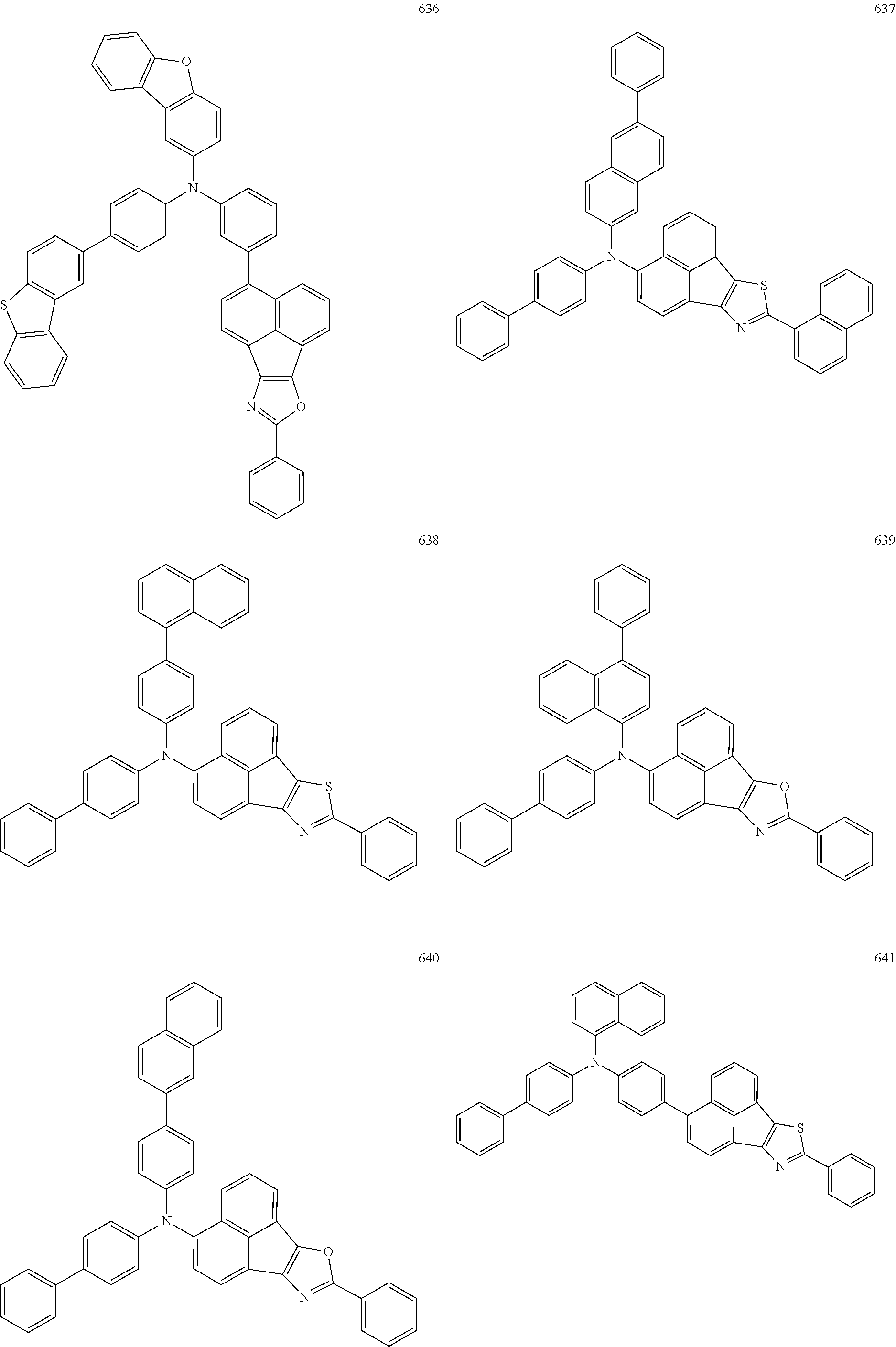

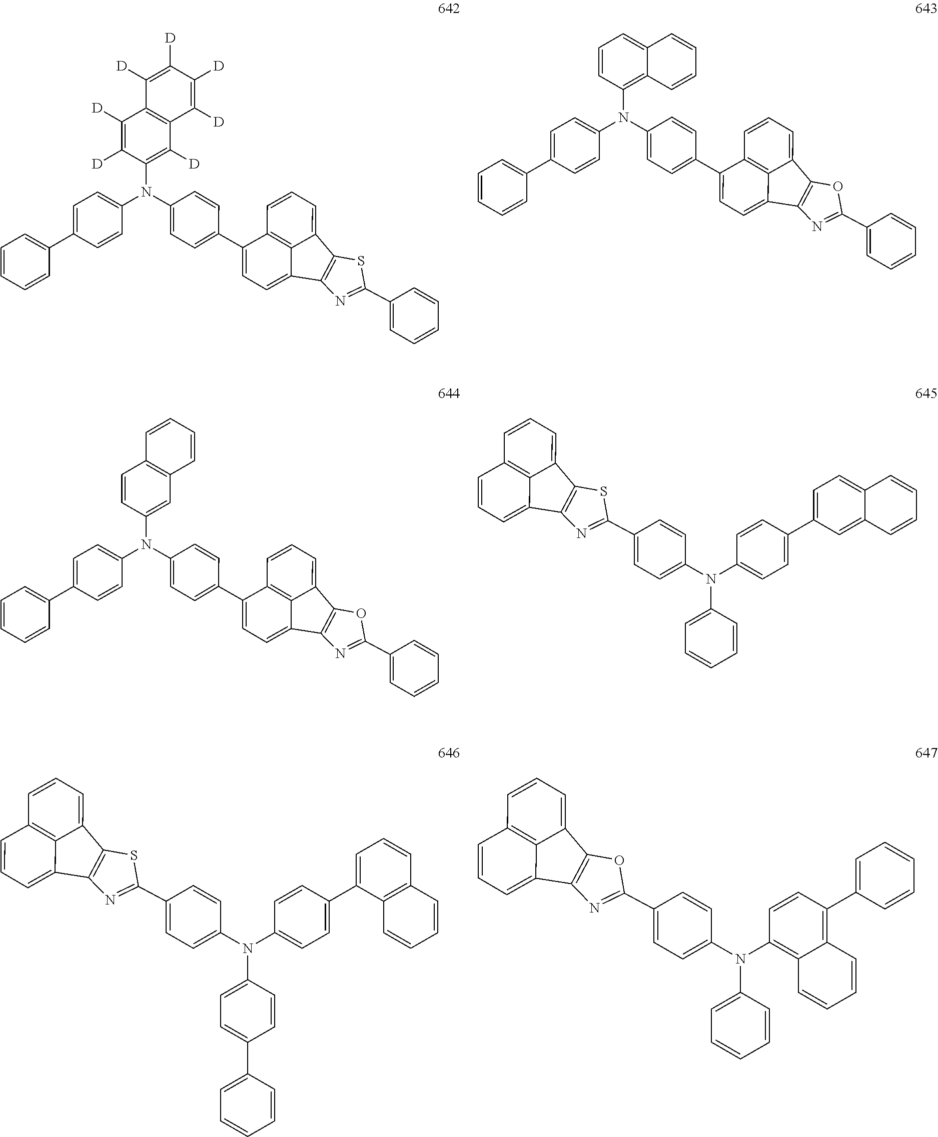

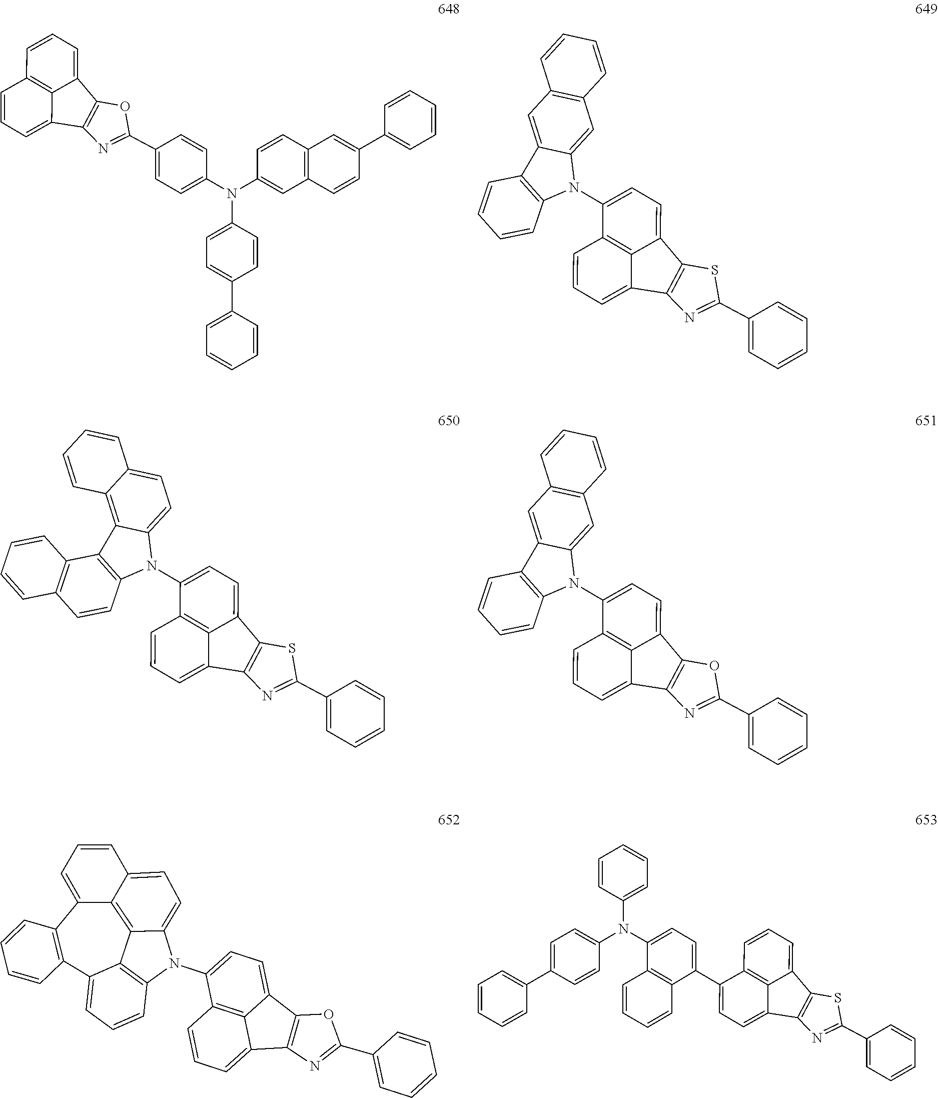 [SMILES: c1ccc(-c2ccc(N(c3ccc(-c4nc5c(o4)-c4cccc6cccc-5c46)cc3)c3ccc4cc(-c5ccccc5)ccc4c3)cc2)cc1 ; c1ccc(-c2nc3c(s2)-c2ccc(-n4c5ccccc5c5cc6ccccc6cc54)c4cccc-3c24)cc1 ; c1ccc(-c2nc3c(s2)-c2ccc(-n4c5ccc6ccccc6c5c5c6ccccc6ccc54)c4cccc-3c24)cc1 ; c1ccc(-c2nc3c(o2)-c2ccc(-n4c5ccccc5c5cc6ccccc6cc54)c4cccc-3c24)cc1 ; c1ccc(-c2nc3c(o2)-c2ccc(-n4c5cccc6c5c5c7c(cccc7ccc54)-c4ccccc4-6)c4cccc-3c24)cc1 ; c1ccc(-c2ccc(N(c3ccccc3)c3ccc(-c4ccc5c6c(cccc46)-c4sc(-c6ccccc6)nc4-5)c4ccccc34)cc2)cc1]

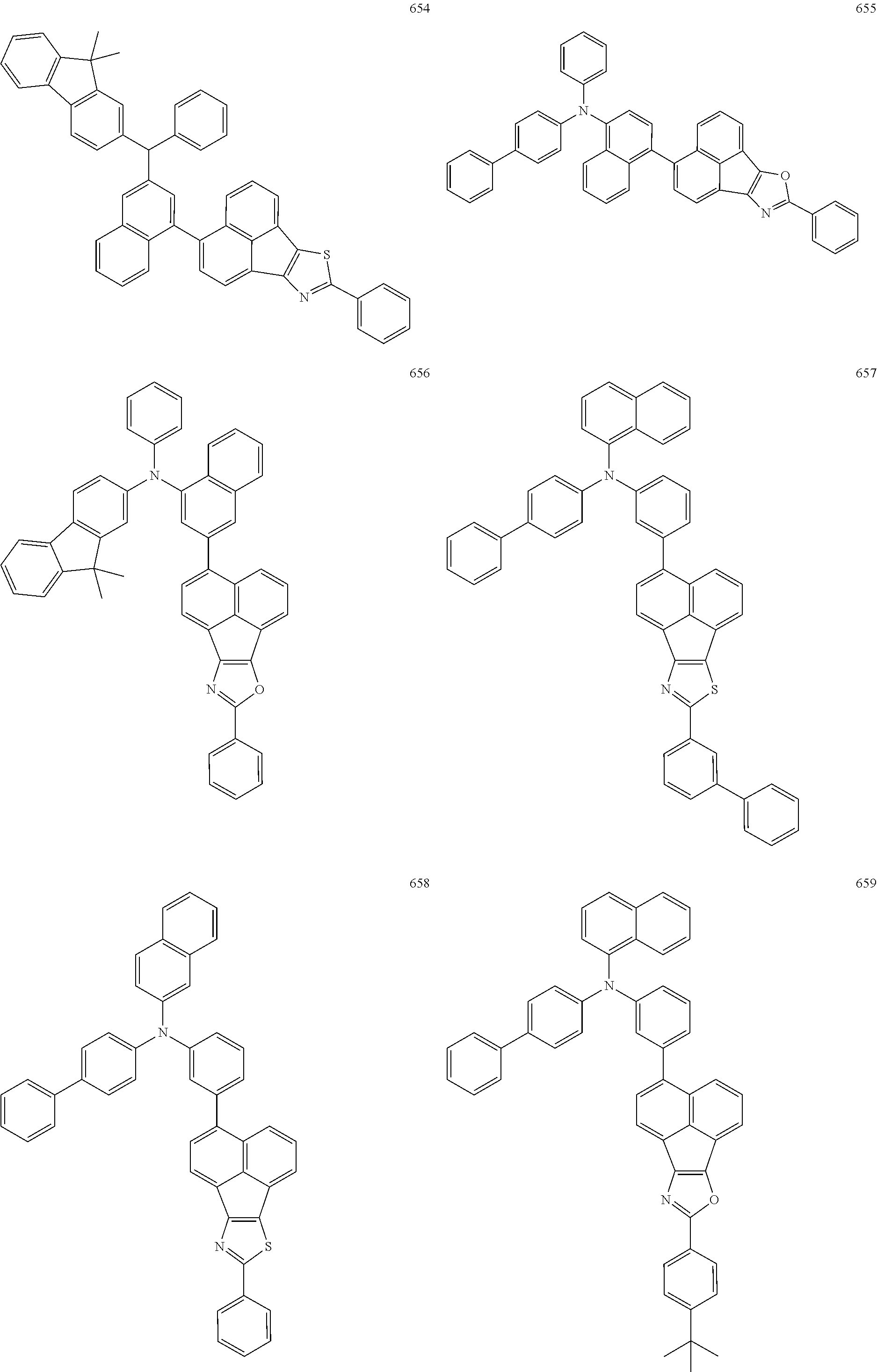

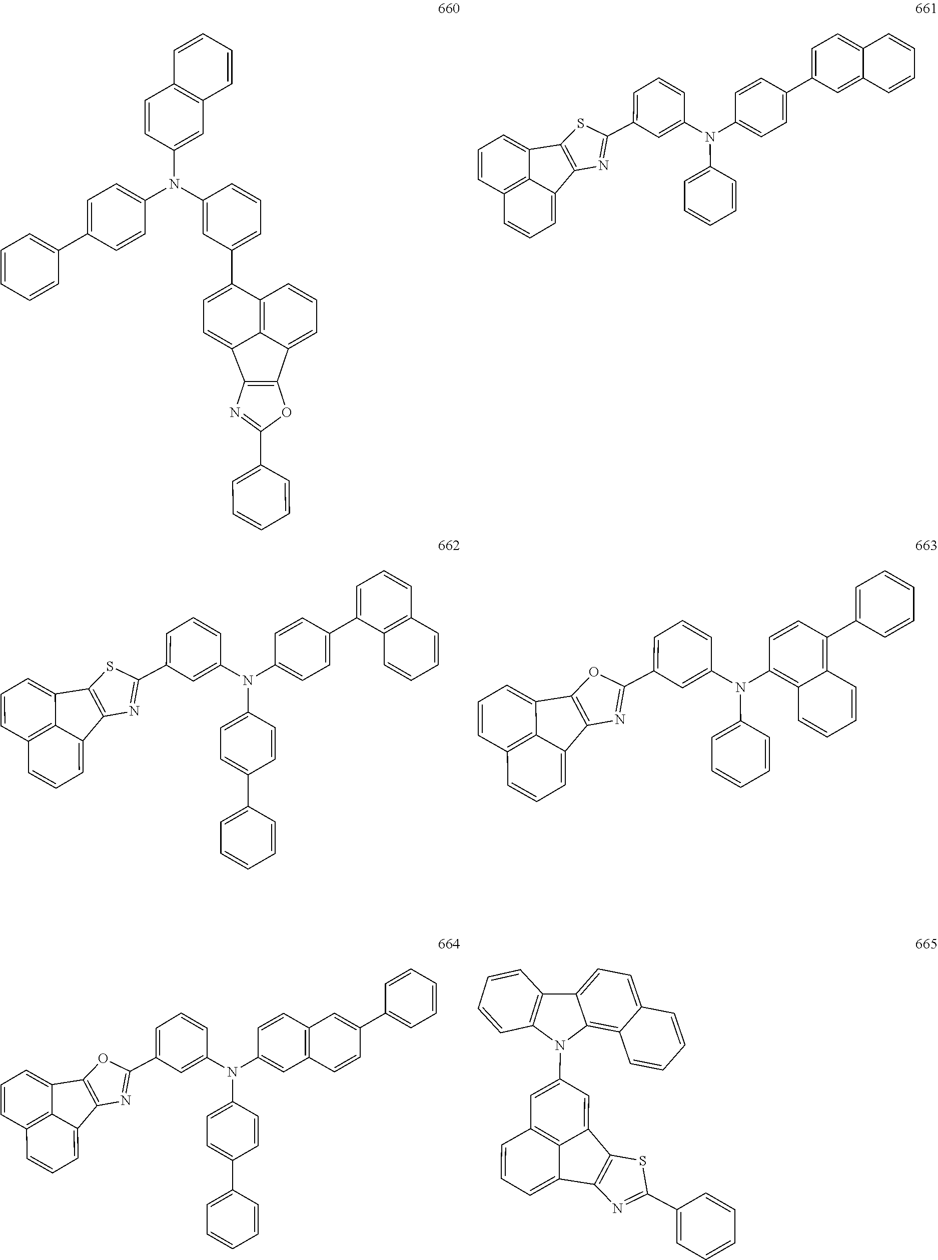

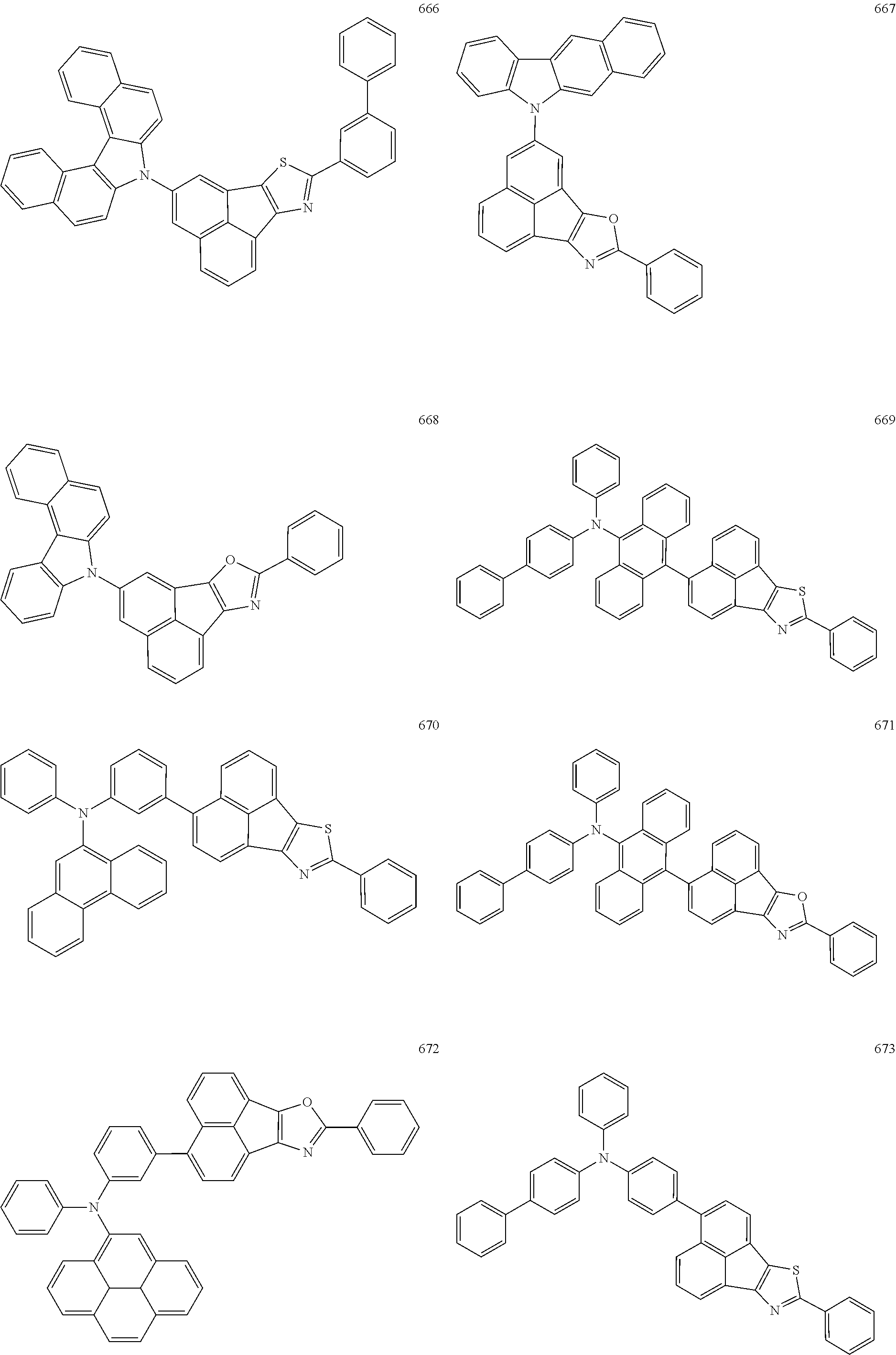

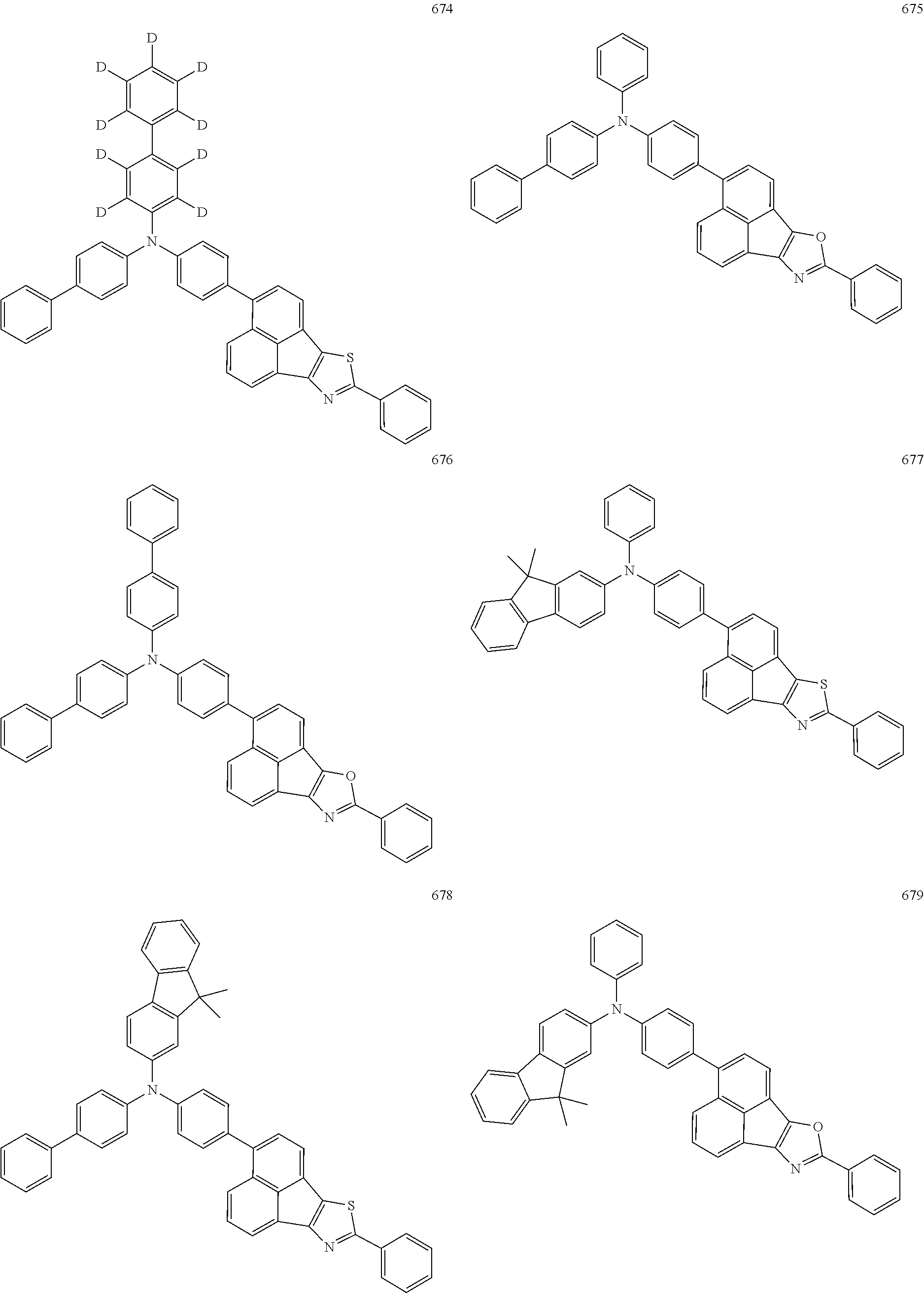

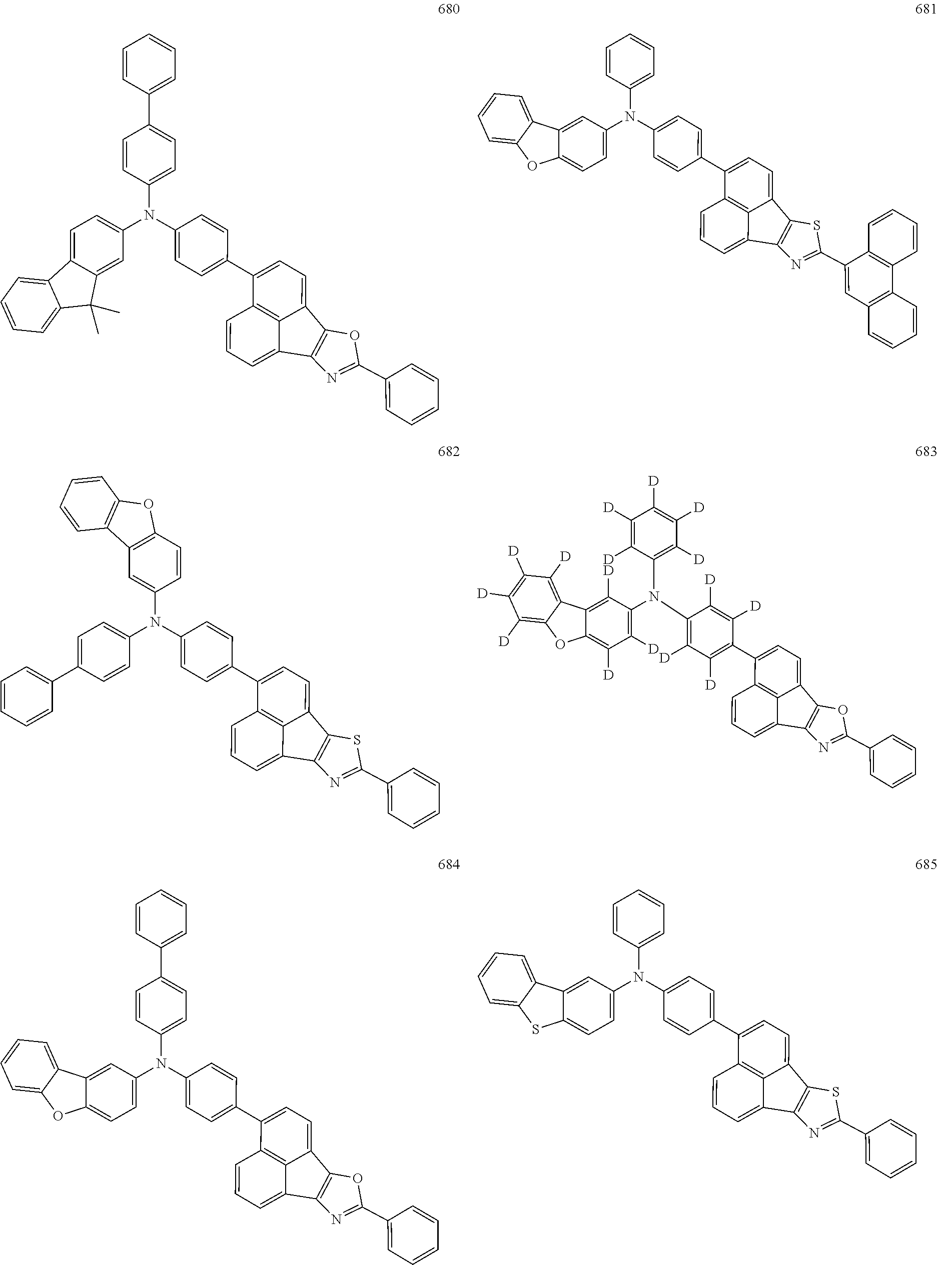

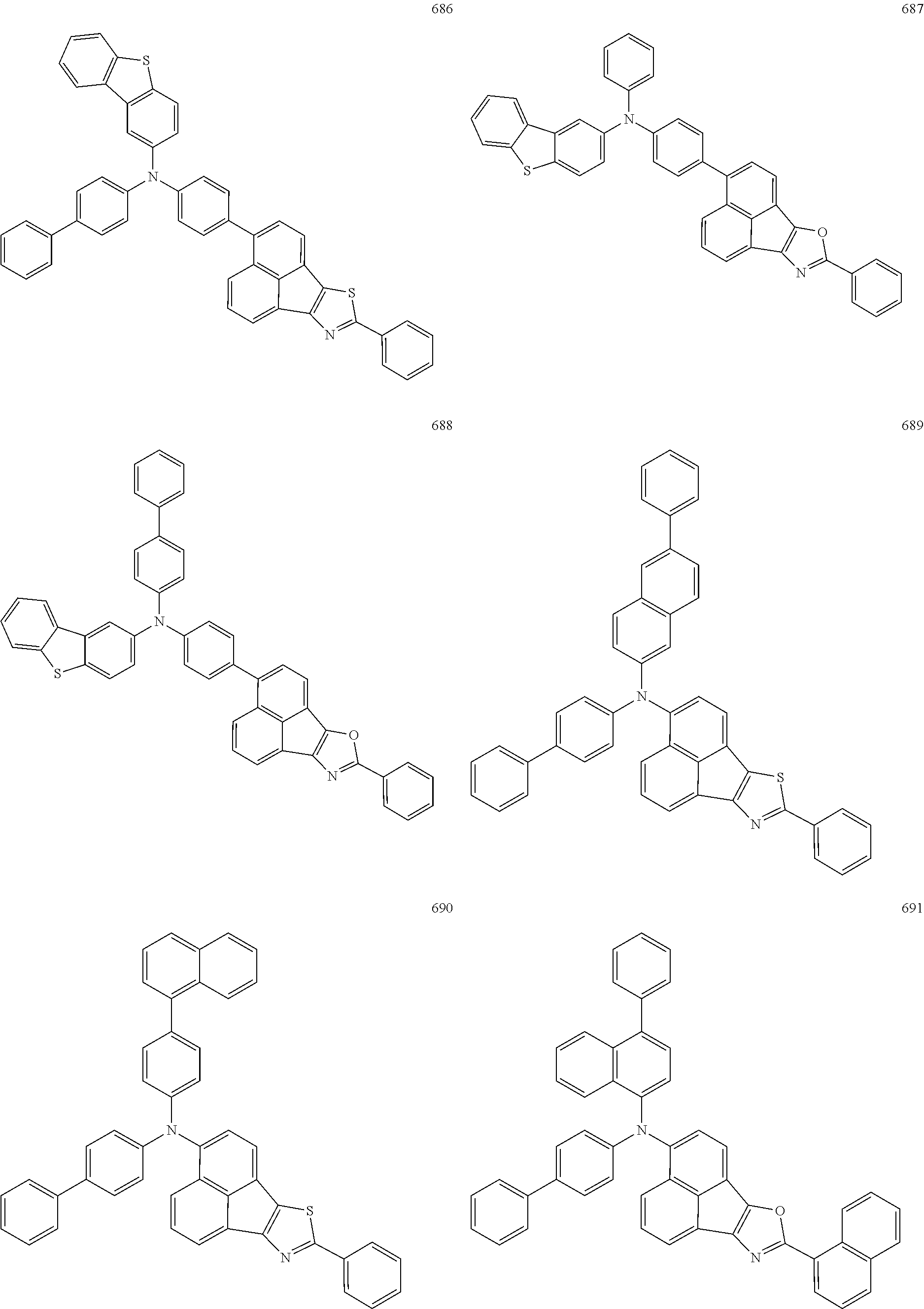

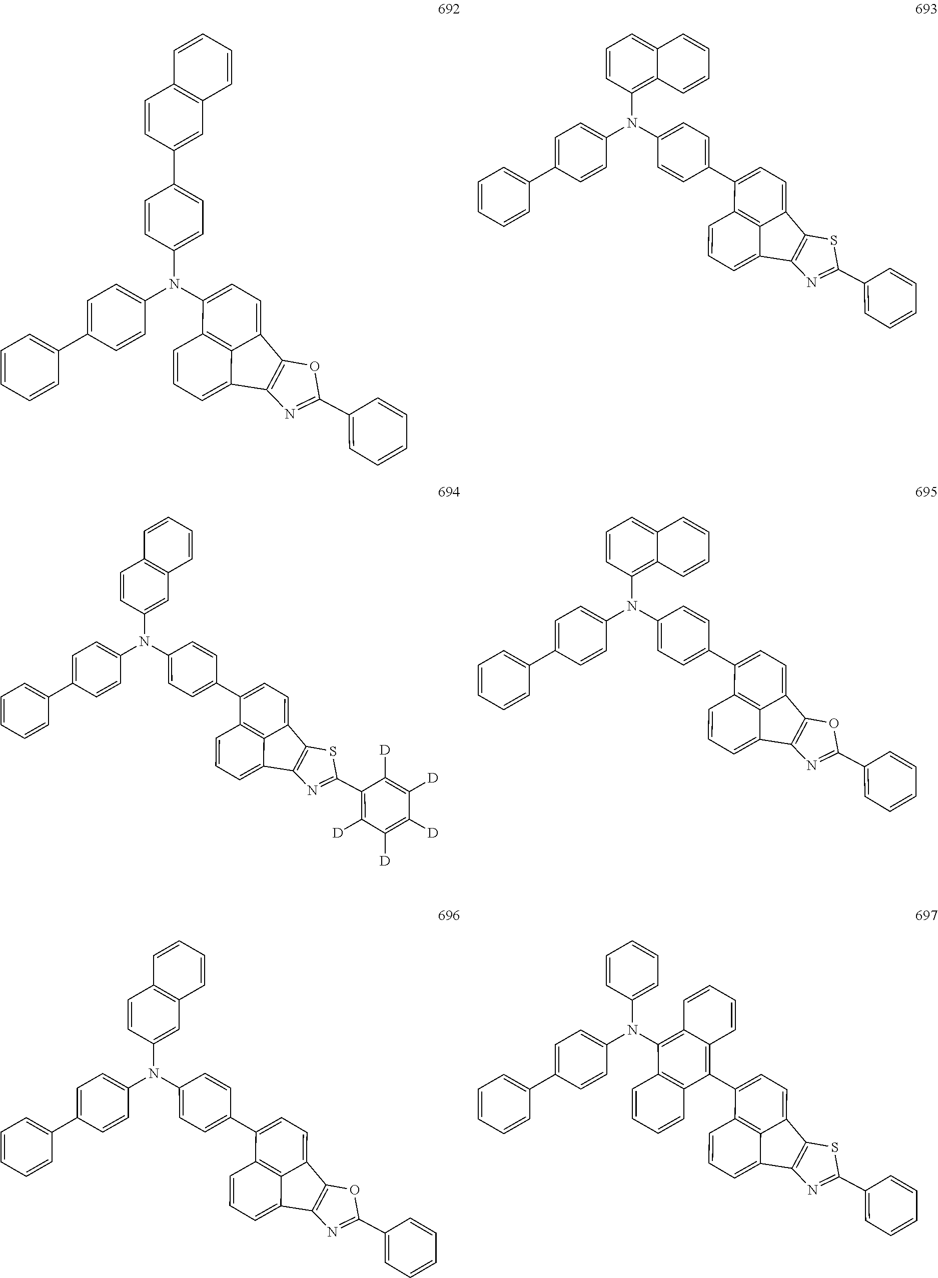

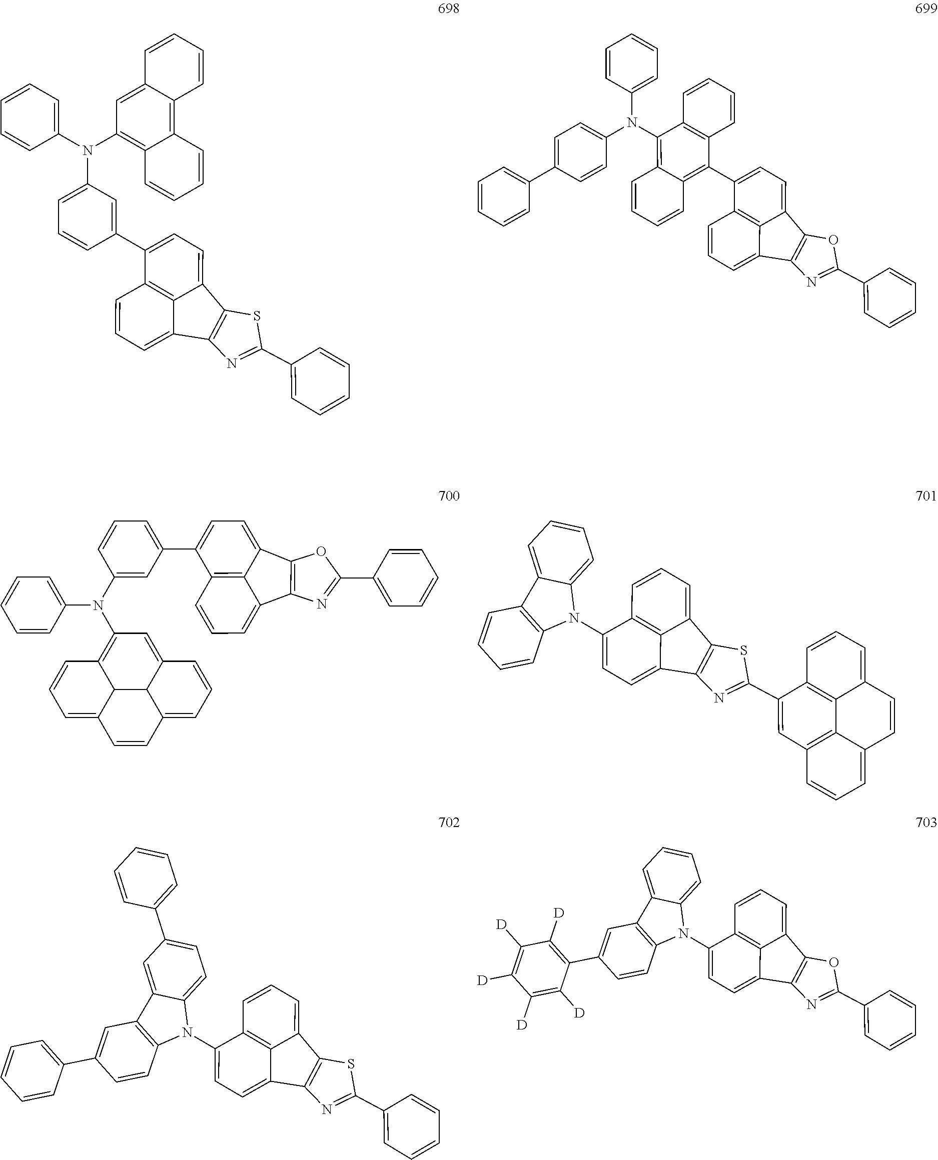

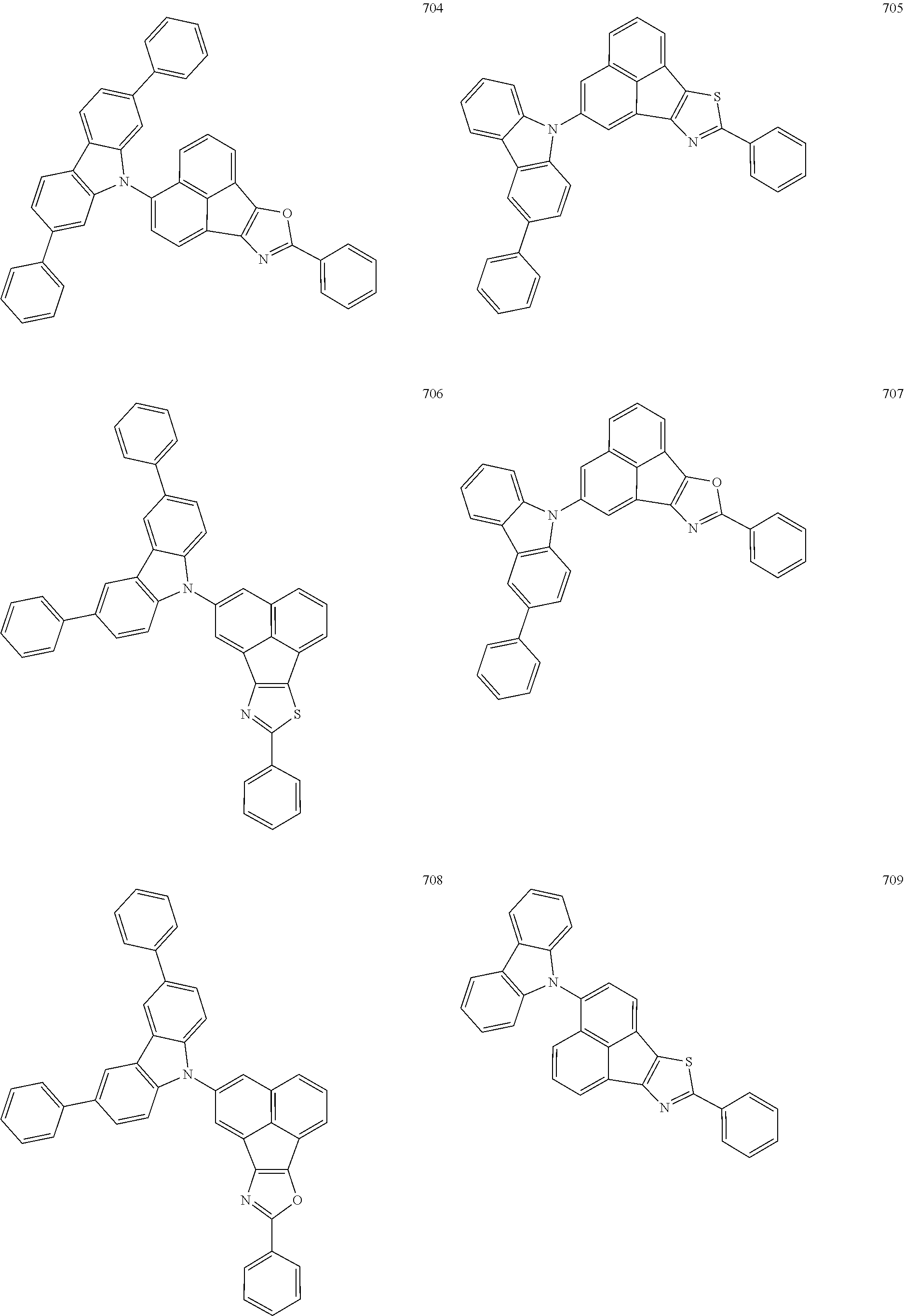

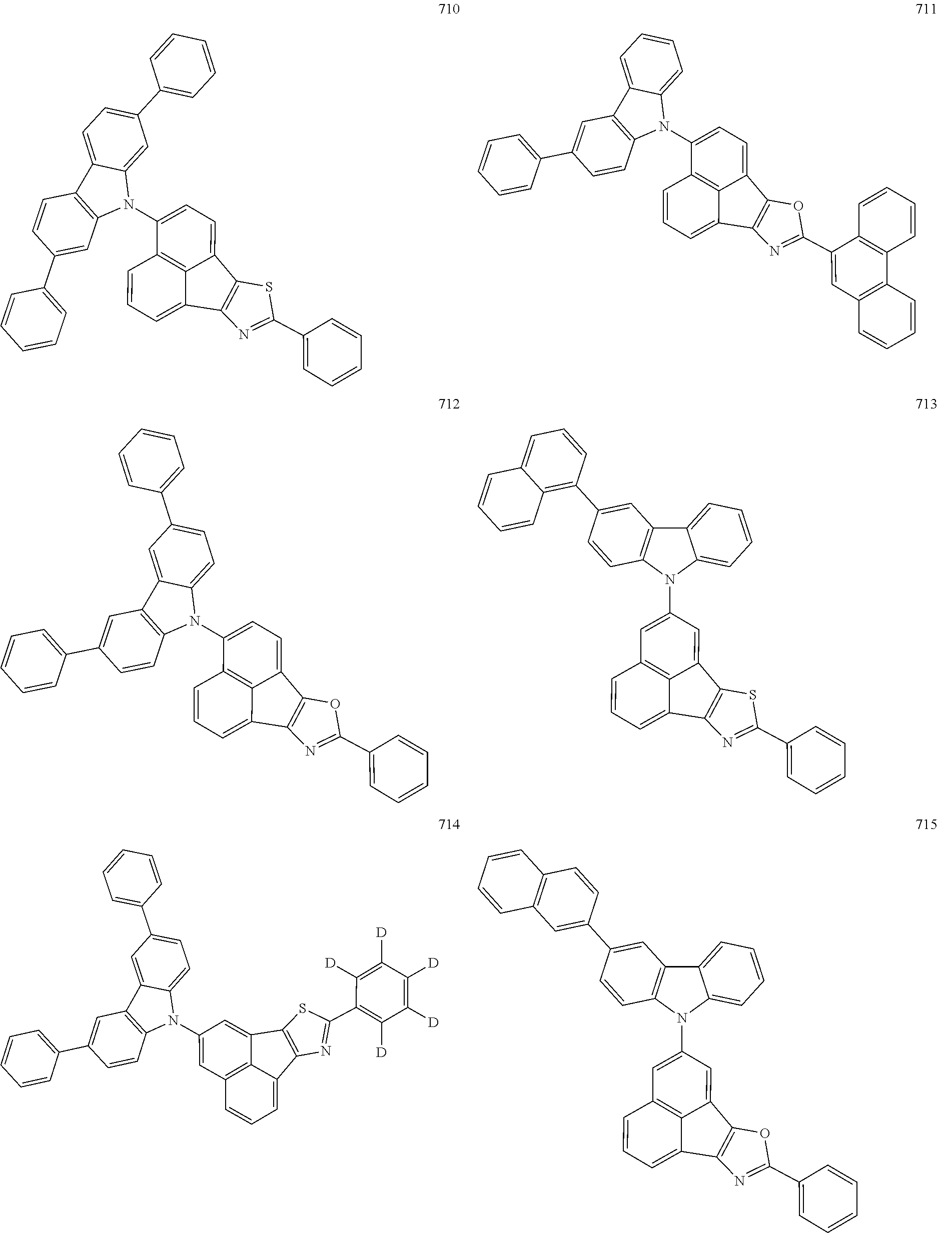

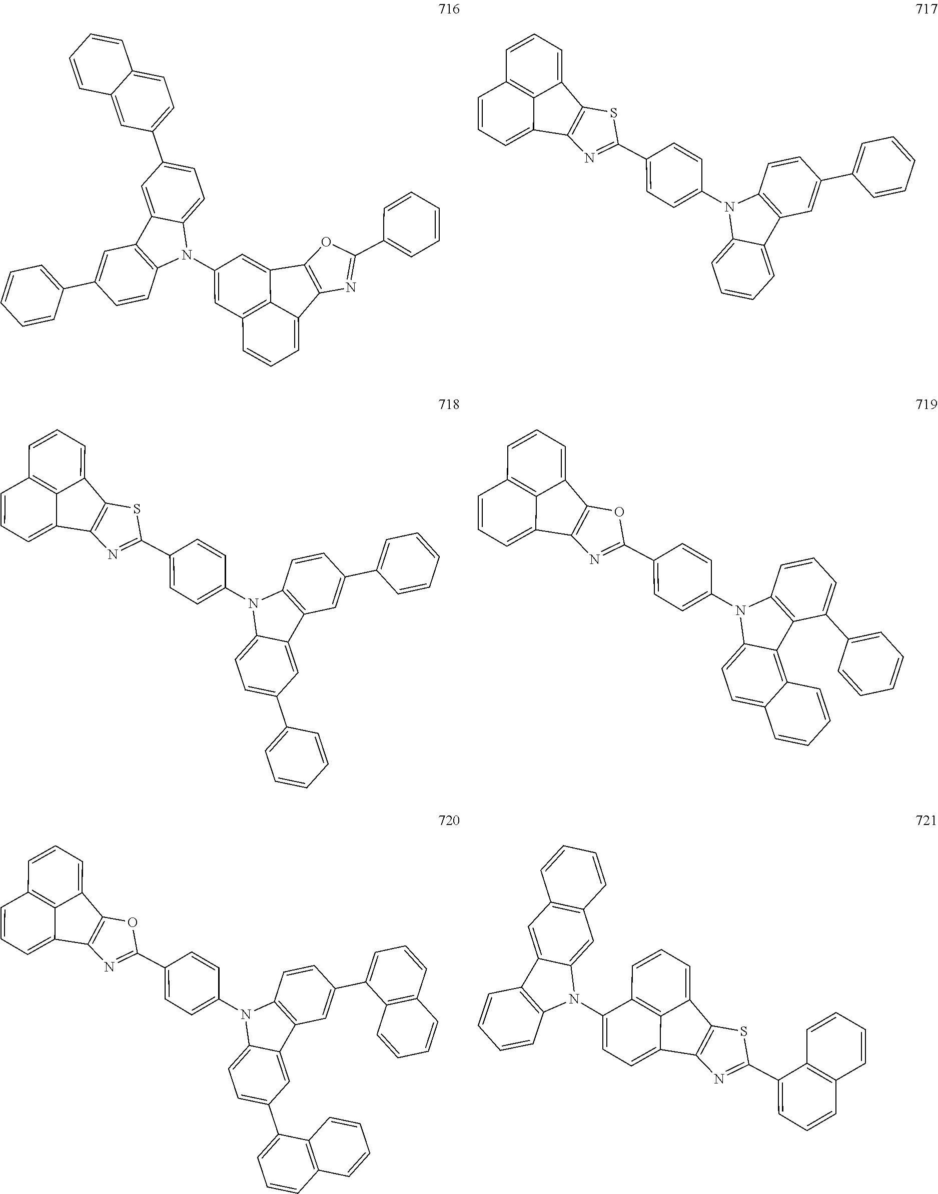

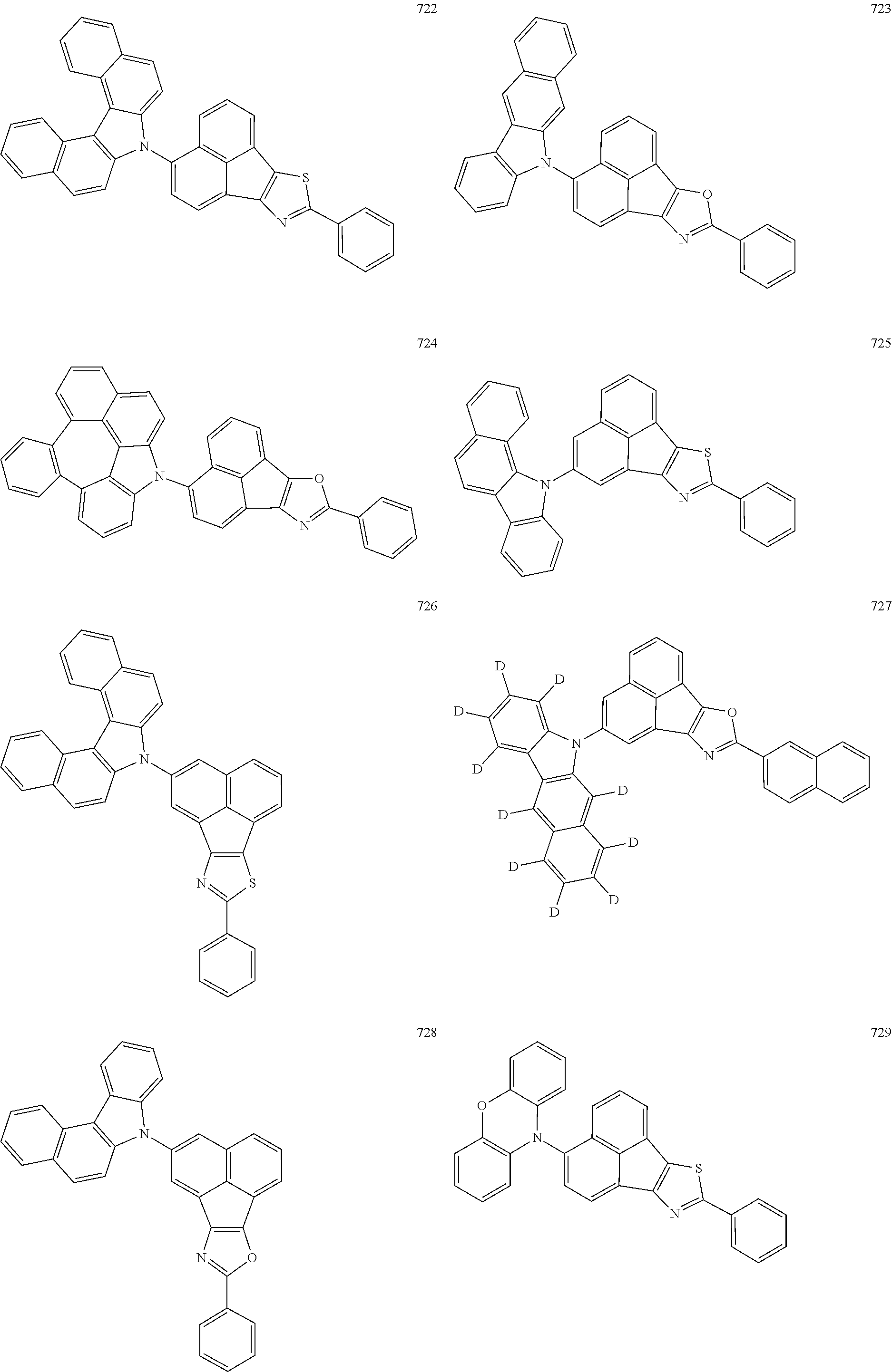

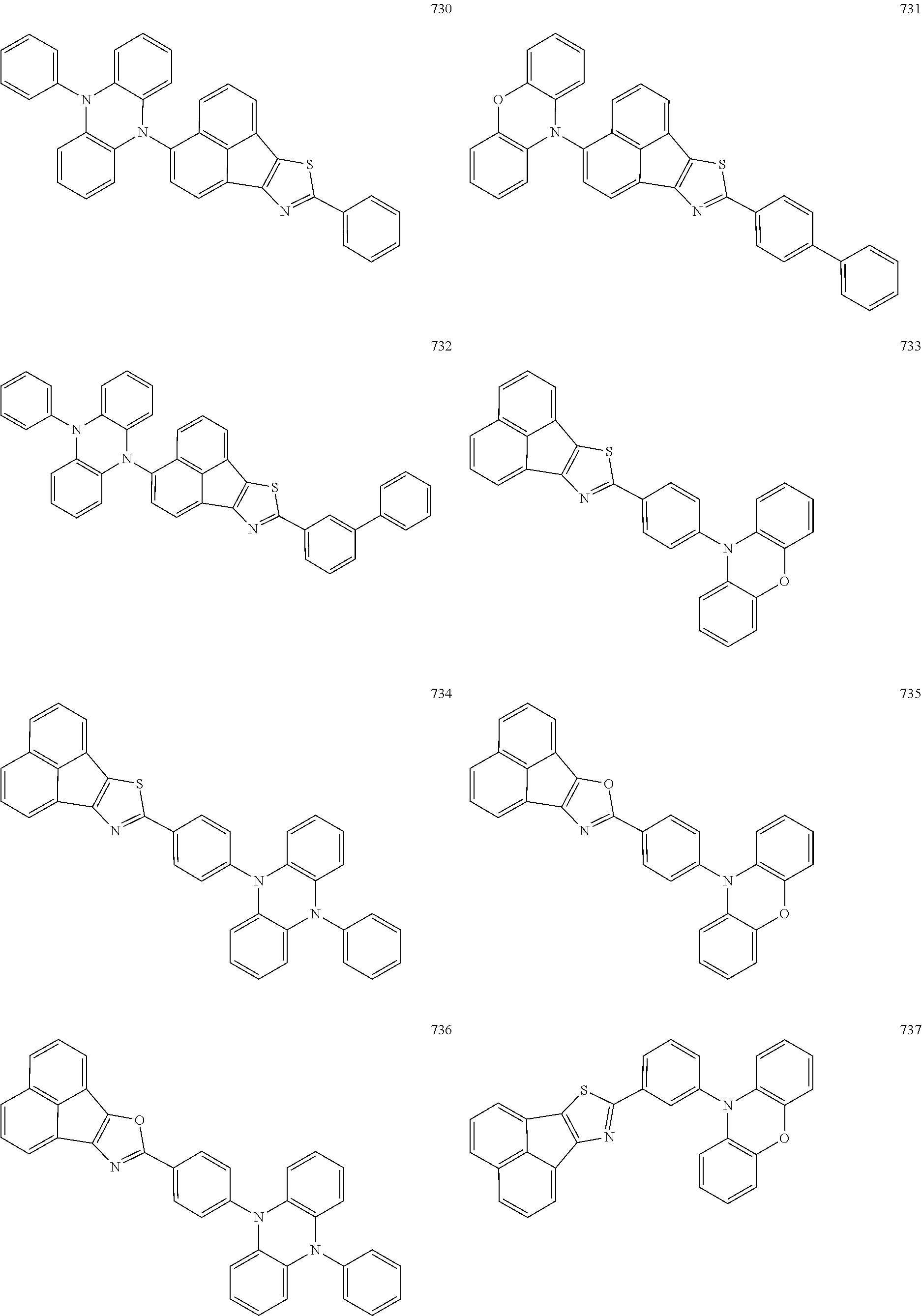

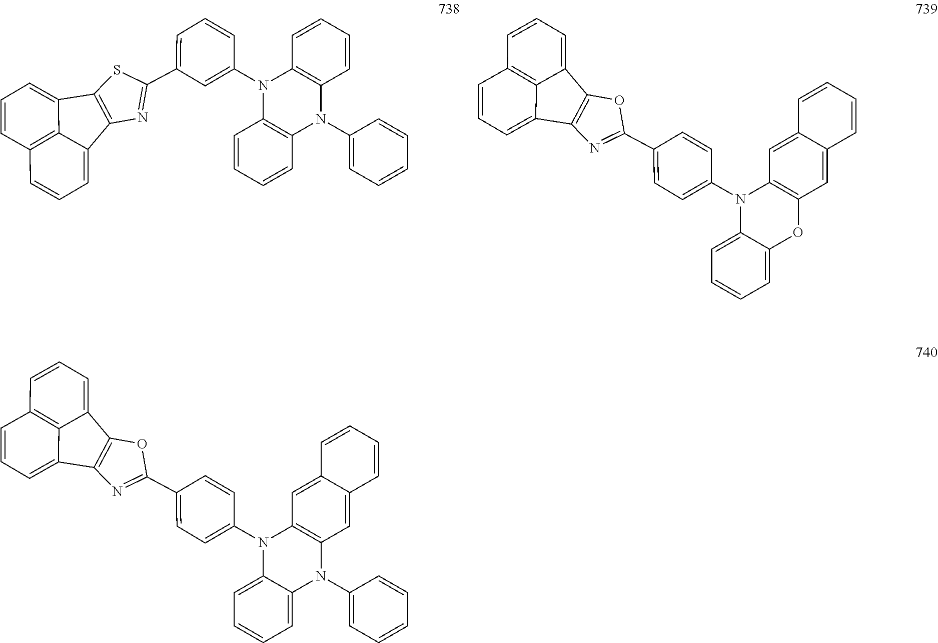 [SMILES: c1ccc(N2c3ccccc3N(c3cccc(-c4nc5c(s4)-c4cccc6cccc-5c46)c3)c3ccccc32)cc1 ; c1ccc2c(c1)Oc1cc3ccccc3cc1N2c1ccc(-c2nc3c(o2)-c2cccc4cccc-3c24)cc1 ; c1ccc(N2c3ccccc3N(c3ccc(-c4nc5c(o4)-c4cccc6cccc-5c46)cc3)c3cc4ccccc4cc32)cc1]

In addition, by introducing various substituents to the structure of Chemical Formula 1, compounds having unique properties of the introduced substituents may be synthesized. For example, by introducing substituents normally used for a hole injection layer material, a hole transport layer material, a light emitting layer material, an electron transport layer material and a charge generation layer material used for manufacturing an organic light emitting device to the core structure, materials satisfying conditions required for each organic material layer may be synthesized.

In addition, by introducing various substituents to the structure of Chemical Formula 1, the energy bandgap may be finely controlled, and meanwhile, properties at interfaces between organic materials may be enhanced, and material applications may become diverse.

Meanwhile, the heterocyclic compound has a high glass transition temperature (Tg), and thereby has excellent thermal stability. Such an increase in the thermal stability becomes an important factor providing driving stability to a device.

The heterocyclic compound according to one embodiment of the present disclosure may be prepared using a multi-step chemical reaction. Some intermediate compounds are prepared first, and from the intermediate compounds, the heterocyclic compound of Chemical Formula 1 may be prepared. More specifically, the heterocyclic compound according to one embodiment of the present disclosure may be prepared based on preparation examples to describe later.

Another embodiment of the present disclosure provides an organic light emitting device including the heterocyclic compound represented by Chemical Formula 1. The “organic light emitting device” may be expressed in terms such as an “organic light emitting diode”, an “OLED”, an “OLED device” and an “organic electroluminescent device”.

In addition, one embodiment of the present disclosure relates to an organic light emitting device comprising:
a first electrode;
a second electrode provided opposite to the first electrode; and
one or more organic material layers provided between the first electrode and the second electrode,
wherein at least one of the one or more organic material layers comprise the heterocyclic compound represented by Chemical Formula 1.

In one embodiment of the present disclosure, the first electrode may be a positive electrode, and the second electrode may be a negative electrode.

In another embodiment, the first electrode may be a negative electrode, and the second electrode may be a positive electrode.

In another embodiment of the present disclosure, the organic light emitting device may be a red organic light emitting device, and the heterocyclic compound represented by Chemical Formula 1 may be used as a material of the red organic light emitting device.

In another embodiment of the present disclosure, the organic light emitting device may be a blue organic light emitting device, and the heterocyclic compound represented by Chemical Formula 1 may be used as a material of the blue organic light emitting device.

In another embodiment of the present disclosure, the organic light emitting device may be a green organic light emitting device, and the heterocyclic compound represented by Chemical Formula 1 may be used as a material of the green organic light emitting device.

In one embodiment of the present disclosure, the organic light emitting device may be a red organic light emitting device, and the heterocyclic compound represented by Chemical Formula 1 may be used as a light emitting layer material of the red organic light emitting device.

In another embodiment of the present disclosure, the organic light emitting device may be a blue organic light emitting device, and the heterocyclic compound represented by Chemical Formula 1 may be used as a light emitting layer material of the blue organic light emitting device.

In another embodiment of the present disclosure, the organic light emitting device may be a green organic light emitting device, and the heterocyclic compound represented by Chemical Formula 1 may be used as a light emitting layer material of the green organic light emitting device.

Specific descriptions on the heterocyclic compound represented by Chemical Formula 1 are the same as the descriptions provided above.

The organic light emitting device of the present disclosure may be manufactured using common organic light emitting device manufacturing methods and materials except that one or more organic material layers are formed using the heterocyclic compound described above.

The heterocyclic compound may be formed into an organic material layer using a solution coating method as well as a vacuum deposition method when the organic light emitting device is manufactured. Herein, the solution coating method means spin coating, dip coating, inkjet printing, screen printing, a spray method, roll coating and the like, but is not limited thereto.

The organic material layer of the organic light emitting device of the present disclosure may be formed in a single layer structure, but may also be formed in a multilayer structure in which two or more organic material layers are laminated. For example, the organic light emitting device of the present disclosure may have a structure including a hole injection layer, an electron blocking layer, a hole transport layer, a light emitting layer, an electron transport layer, a hole blocking layer, an electron injection layer and the like as the organic material layers. However, the structure of the organic light emitting device is not limited thereto, and may include a smaller number of organic material layers.

In the organic light emitting device of the present disclosure, the organic material layer includes a light emitting layer, and the light emitting layer may include the heterocyclic compound represented by Chemical Formula 1. When the heterocyclic compound is used in the light emitting layer, driving efficiency and lifetime of the organic light emitting device may become superior since strong charge transfer is possible by spatially separating HOMO (highest occupied molecular orbital) and LUMO (lowest unoccupied molecular orbital).

In one embodiment of the present disclosure, the organic material layer includes the heterocyclic compound represented by Chemical Formula 1, and a phosphorescent dopant may be used therewith.

As the phosphorescent dopant material, those known in the art may be used. For example, phosphorescent dopant materials represented by LL′MX′, LL′L″M, LMX′X″, L₂MX′ and L₃M may be used, however, the scope of the present disclosure is not limited by these examples.
M may be iridium, platinum, osmium or the like.
L is an anionic bidentate ligand coordinated to M by sp² carbon and heteroatom, and X may function to trap electrons or holes. Nonlimiting examples of L may include 2-(1-naphthyl)benzoxazole, 2-phenylbenzoxazole, 2-phenylbenzothiazole, 7,8-benzoquinoline, phenylpyridine, benzothiophenylpyridine, 3-methoxy-2-phenylpyridine, thiophenylpyridine, tolylpyridine and the like. Nonlimiting examples of X′ and X″ may include acetylacetonate (acac), hexafluoroacetylacetonate, salicylidene, picolinate, 8-hydroxyquinolinate and the like.

Specific examples of the phosphorescent dopant are shown below, however, the phosphorescent dopant is not limited to these examples.

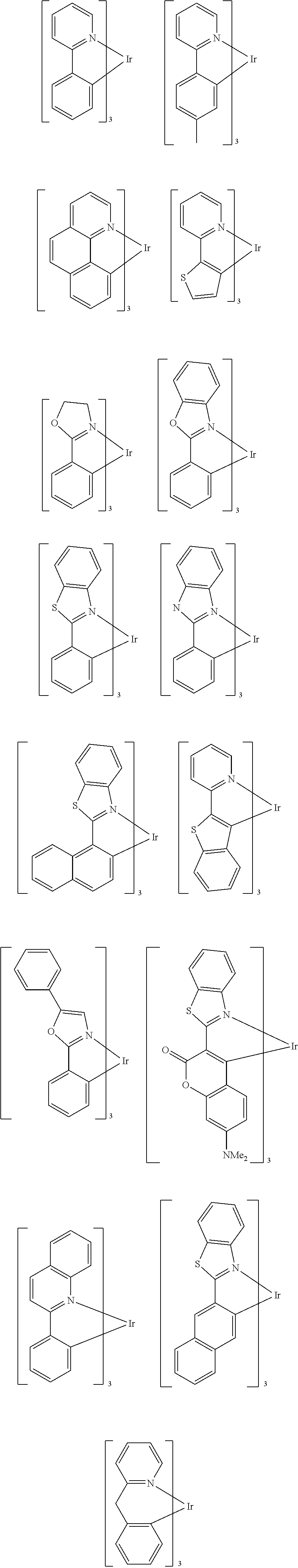

In one embodiment of the present disclosure, the organic material layer comprises the heterocyclic compound represented by Chemical Formula 1, and an iridium-based dopant may be used therewith.

In one embodiment of the present disclosure, as the iridium-based dopant, (piq)₂(Ir) (acac) may be used as a red phosphorescent dopant.

In one embodiment of the present disclosure, a content of the dopant may be from 1% to 15%, preferably from 2% to 10% and more preferably from 3% to 7% based on the total weight of the light emitting layer.

In the organic light emitting device according to one embodiment of the present disclosure, the organic material layer includes an electron injection layer or an electron transport layer, and the electron injection layer or the electron transport layer may include the heterocyclic compound represented by Chemical Formula 1.

In the organic light emitting device according to another embodiment of the present disclosure, the organic material layer includes an electron blocking layer or a hole blocking layer, and the electron blocking layer or the hole blocking layer may include the heterocyclic compound represented by Chemical Formula 1.

In the organic light emitting device according to another embodiment, the organic material layer includes an electron transport layer, a light emitting layer or a hole blocking layer, and the electron transport layer, the light emitting layer or the hole blocking layer may include the heterocyclic compound represented by Chemical Formula 1.

In the organic light emitting device according to another embodiment, the organic material layer includes a light emitting layer, and the light emitting layer may include the heterocyclic compound represented by Chemical Formula 1.

In the organic light emitting device according to another embodiment, the organic material layer includes a light emitting layer, the light emitting layer includes a host material, and the host material may include the heterocyclic compound represented by Chemical Formula 1.

In the organic light emitting device according to another embodiment, the light emitting layer may include two or more host materials, and at least one of the host materials may include the heterocyclic compound represented by Chemical Formula 1.

In the organic light emitting device according to another embodiment, two or more host materials may be pre-mixed and used in the light emitting layer, and at least one of the two or more host materials may include the heterocyclic compound represented by Chemical Formula 1.

The pre-mixing means, before depositing the two or more host materials on the organic material layer, mixing the materials first in one source of supply.

The organic light emitting device according to one embodiment of the present disclosure may further include one, two or more layers selected from the group consisting of a light emitting layer, a hole injection layer, a hole transport layer, an electron injection layer, an electron blocking layer and a hole blocking layer.

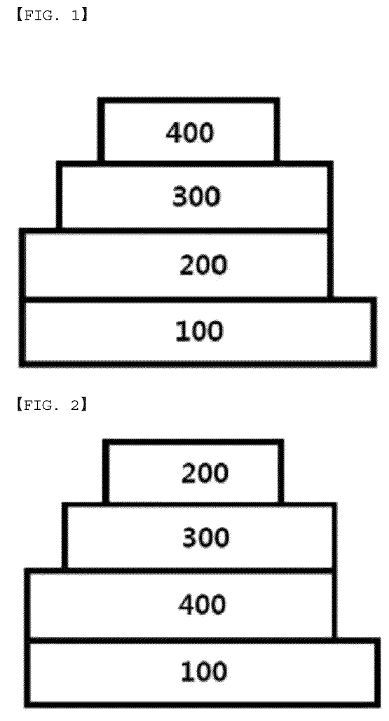

FIG. 1 to FIG. 4 illustrate a lamination order of electrodes and organic material layers of the organic light emitting device according to one embodiment of the present disclosure. However, the scope of the present application is not limited by these diagrams, and structures of organic light emitting devices known in the art may also be applied to the present application.

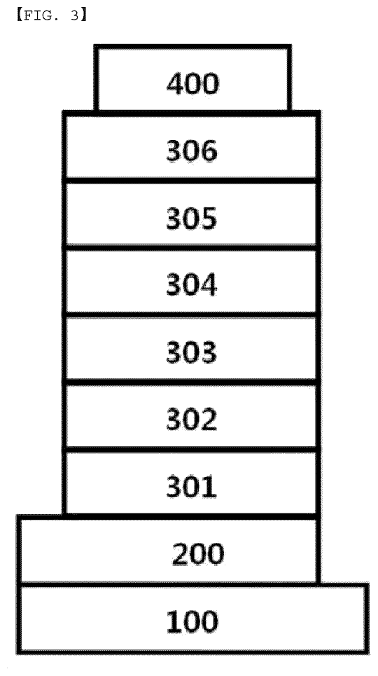

FIG. 1 illustrates an organic light emitting device in which a positive electrode 200, an organic material layer 300 and a negative electrode 400 are sequentially laminated on a substrate 100. However, the structure of the organic light emitting device is not limited only to such a structure, and as illustrated in FIG. 2, an organic light emitting device in which a negative electrode, an organic material layer and a positive electrode are sequentially laminated on a substrate may also be obtained.

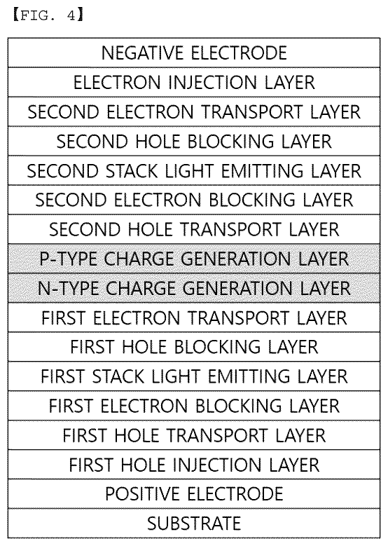

FIG. 3 illustrates a case of the organic material layer being a multilayer. An organic light emitting device according to FIG. 3 includes a hole injection layer 301, a hole transport layer 302, a light emitting layer 303, a hole blocking layer 304, an electron transport layer 305 and an electron injection layer 306. However, the scope of the present application is not limited by such a lamination structure, and as necessary, the layers other than the light emitting layer may not be included, and other necessary functional layers may be further added.

As the organic light emitting device according to one embodiment of the present application, an organic light emitting device having a 2-stack tandem structure is schematically illustrated in FIG. 4.

Herein, the first electron blocking layer, the first hole blocking layer, the second hole blocking layer and the like described in FIG. 4 may not be included in some cases.

One embodiment of the present disclosure provides a method for manufacturing an organic light emitting device, the method comprising the steps of:
preparing a substrate;
forming a first electrode on the substrate;
forming one or more organic material layers on the first electrode; and
forming a second electrode on the one or more organic material layers,
wherein the steps of forming the one or more organic material layers includes a step of forming the one or more organic material layers using the composition for an organic material layer according to one embodiment of the present disclosure.

The organic material layer including the heterocyclic compound represented by Chemical Formula 1 may further include other materials as necessary.

In the organic light emitting device according to one embodiment of the present disclosure, materials other than the heterocyclic compound represented by Chemical Formula 1 are illustrated below, however, these are for illustrative purposes only and not for limiting the scope of the present application, and these materials may be replaced by materials known in the art.

As the positive electrode material, materials each having a relatively large work function may be used, and transparent conductive oxides, metals, conductive polymers or the like may be used. Specific examples of the positive electrode material include metals such as vanadium, chromium, copper, zinc and gold, or alloys thereof; metal oxides such as zinc oxide, indium oxide, indium tin oxide (ITO) and indium zinc oxide (IZO); combinations of metals and oxides such as ZnO:Al or SnO₂:Sb; conductive polymers such as poly(3-methylthiophene), poly[3,4-(ethylene-1,2-dioxy)thiophene] (PEDOT), polypyrrole and polyaniline, and the like, but are not limited thereto.

As the negative electrode material, materials each having a relatively small work function may be used, and metals, metal oxides, conductive polymers or the like may be used. Specific examples of the negative electrode material include metals such as magnesium, calcium, sodium, potassium, titanium, indium, yttrium, lithium, gadolinium, aluminum, silver, tin and lead, or alloys thereof; multilayer structure materials such as LiF/Al or LiO₂/Al, and the like, but are not limited thereto.

As the hole injection layer material, known hole injection layer materials may be used, and for example, phthalocyanine compounds such as copper phthalocyanine disclosed in U.S. Pat. No. 4,356,429, or starburst-type amine derivatives such as tris(4-carbazoyl-9-ylphenyl)amine (TCTA), 4,4′,4″-tris[phenyl(m-tolyl)amino]triphenylamine (m-MTDATA) or 1,3,5-tris[4-(3-methylphenylphenylamino)phenyl]benzene (m-MTDAPB) described in the literature [Advanced Material, 6, p. 677 (1994)], conductive polymers having solubility such as polyaniline/dodecylbenzenesulfonic acid or poly(3,4-ethylenedioxythiophene)/poly(4-styrenesulfonate), polyaniline/camphor sulfonic acid or polyaniline/poly(4-styrenesulfonate), and the like, may be used.

As the hole transport layer material, pyrazoline derivatives, arylamine-based derivatives, stilbene derivatives, triphenyldiamine derivatives and the like may be used, and low molecular or high molecular materials may also be used.

As the electron transport layer material, metal complexes of oxadiazole derivatives, anthraquinodimethane and derivatives thereof, benzoquinone and derivatives thereof, naphthoquinone and derivatives thereof, anthraquinone and derivatives thereof, tetracyanoanthraquinodimethane and derivatives thereof, fluorenone derivatives, diphenyldicyanoethylene and derivatives thereof, diphenoquinone derivatives, 8-hydroxyquinoline and derivatives thereof, and the like, may be used, and high molecular materials as well as low molecular materials may also be used.

As examples of the electron injection layer material, LiF is typically used in the art, however, the present application is not limited thereto.

As the light emitting layer material, red, green or blue light emitting materials may be used, and as necessary, two or more light emitting materials may be mixed and used. Herein, the two or more light emitting materials may be deposited as individual sources of supply or pre-mixed and deposited as one source of supply when used. In addition, fluorescent materials may also be used as the light emitting layer material, however, phosphorescent materials may also be used. As the light emitting layer material, materials emitting light alone by binding holes and electrons injected from a positive electrode and a negative electrode, respectively, may be used, however, materials having a host material and a dopant material involving together in light emission may also be used.

When hosts of the light emitting layer material are mixed and used, same series hosts may be mixed and used, or different series hosts may be mixed and used. For example, any two or more types of materials among n-type host materials and p-type host materials may be selected and used as a host material of a light emitting layer.

The organic light emitting device according to one embodiment of the present disclosure may be a top-emission type, a bottom-emission type or a dual-emission type depending on the materials used.

The heterocyclic compound according to one embodiment of the present disclosure may also be used in an organic electronic device including an organic solar cell, an organic photo conductor, an organic transistor and the like under a principle similar to that in the organic light emitting device.

Hereinafter, preferred examples are provided to help to understand the present disclosure, however, the following examples are only provided to more readily understand the present disclosure, and the present disclosure is not limited thereto.

### PREPARATION EXAMPLE

### Preparation Example 1. Preparation of Intermediate A

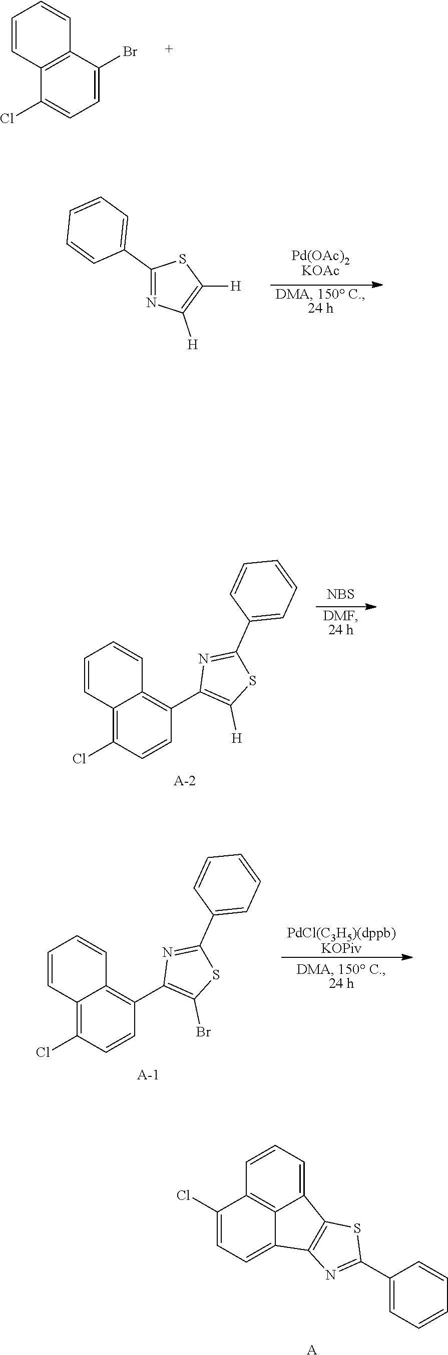

### Preparation Example 1-1. Preparation of Intermediate A-2

1-bromo-4-chloronaphthalene (100 g, 414 mmol), 2-phenylthiazole (100 g, 621 mmol), palladium acetate (Pd(OAc)₂) (4.65 g, 20.7 mmol), potassium acetate (KOAc) (81.3 g, 828 mmol) and dimethylacetamide (DMA) (1 L) were introduced to a 2 L round bottom flask, and the mixture was stirred for 24 hours at a temperature of 150° C.

After the reaction was completed, the result was filtered under reduced pressure using dichloromethane (DCM) to remove the salt, and the solvent was removed using a rotary evaporator for concentration.

After that, the reaction material was separated by column chromatography (diethyl ether:hexane=1:10 (volume ratio)) to obtain Intermediate A-2 (117 g, 364 mmol, yield 88%).

### Preparation Example 1-2. Preparation of Intermediate A-1

Intermediate A-2 (117 g, 364 mmol), N-bromosuccinimide (NBS) (130 g, 728 mmol) and dimethylformide (DMF) (1.2 L) were introduced to a 2 L round bottom flask, and the mixture was stirred for 24 hours.

After the reaction was completed, the result was concentrated and separated by column chromatography (diethyl ether:hexane=1:5 (volume ratio)) to obtain Intermediate A-1 (88.9 g, 222 mmol, yield 61%).

### Preparation Example 1-3. Preparation of Intermediate A

Intermediate A-1 (88.9 g, 222 mmol), KOPiv (62.3 g, 444 mmol), dimethylacetamide (DMA) (890 mL) and [1,4-bis(diphenylphosphino)butane] (η³-allyl)palladium(II) chloride (PdCl(C₃H₅) (dppb)) (6.76 g, 11.1 mmol) were introduced to a 2 L round bottom flask in consecutive order, and the mixture was stirred for 24 hours at a temperature of 150° C.

After the reaction was completed, the result was filtered under reduced pressure using dichloromethane (DCM) to remove the salt, and the solvent was removed using a rotary evaporator for concentration.

After that, the reaction material was separated by column chromatography (diethyl ether:hexane=2:3 (volume ratio)) to obtain Intermediate A (13.5 g, 42.2 mmol, yield 19%).

### Preparation Example 2. Preparation of Intermediate B

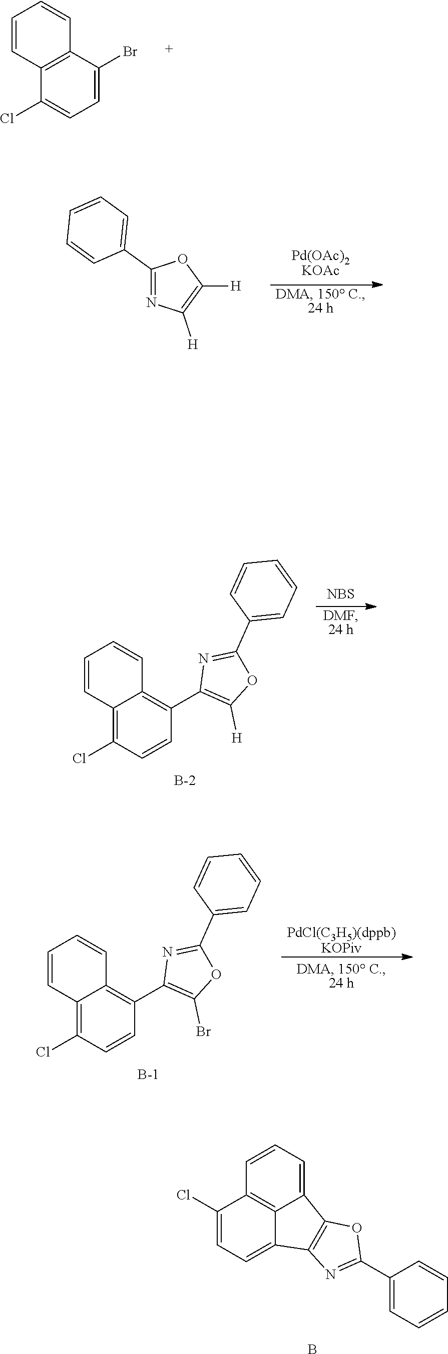

### Preparation Example 2-1. Preparation of Intermediate B-2

1-bromo-4-chloronaphthalene (100 g, 414 mmol), 2-phenyloxazole (90.2 g, 621 mmol), palladium acetate (Pd(OAc)₂) (4.65 g, 20.7 mmol), potassium acetate (KOAc) (81.3 g, 828 mmol) and dimethylacetamide (DMA) (1 L) were introduced to a 2 L round bottom flask, and the mixture was stirred for 24 hours at a temperature of 150° C.

After the reaction was completed, the result was filtered under reduced pressure using dichloromethane (DCM) to remove the salt, and the solvent was removed using a rotary evaporator for concentration.

After that, the reaction material was separated by column chromatography (diethyl ether:hexane=1:10 (volume ratio)) to obtain Intermediate B-2 (113 g, 373 mmol, yield 90%).

### Preparation Example 2-2. Preparation of Intermediate B-1

Intermediate B-2 (113 g, 373 mmol), N-bromosuccinimide (NBS) (133 g, 746 mmol) and dimethylformide (DMF) (1.1 L) were introduced to a 2 L round bottom flask, and the mixture was stirred for 24 hours.

After the reaction was completed, the result was concentrated and separated by column chromatography (diethyl ether:hexane=1:5 (volume ratio)) to obtain Intermediate B-1 (88.1 g, 229 mmol, yield 62%).

### Preparation Example 2-3. Preparation of Intermediate B

Intermediate B-1 (88.1 g, 229 mmol), KOPiv (64.2 g, 458 mmol), dimethylacetamide (DMA) (890 mL) and [1,4-bis(diphenylphosphino)butane] (η³-allyl)palladium(II) chloride (PdCl(C₃H₅) (dppb)) (6.98 g, 11.5 mmol) were introduced to a 2 L round bottom flask in consecutive order, and the mixture was stirred for 24 hours at a temperature of 150° C.

After the reaction was completed, the result was filtered under reduced pressure using dichloromethane (DCM) to remove the salt, and the solvent was removed using a rotary evaporator for concentration.

After that, the reaction material was separated by column chromatography (diethyl ether:hexane=2:3 (volume ratio)) to obtain Intermediate B (14.6 g, 48.1 mmol, yield 21%).

### Preparation Example 3. Preparation of Intermediate C

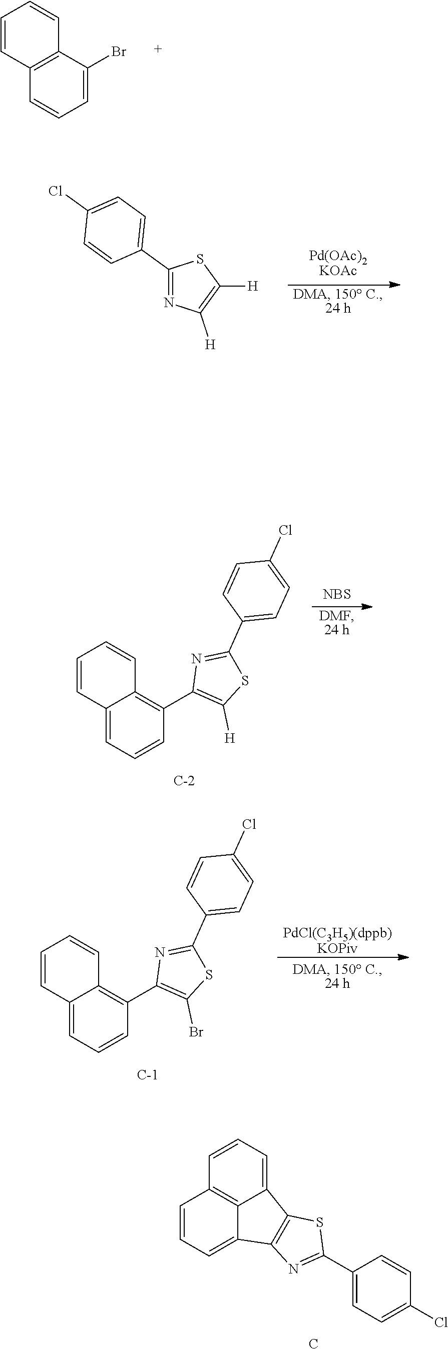

### Preparation Example 3-1. Preparation of Intermediate C-2

1-bromonaphthalene (100 g, 483 mmol), 2-chlorophenylthiazole (142 g, 724 mmol), palladium acetate (Pd(OAc)₂) (5.42 g, 24.2 mmol), potassium acetate (KOAc) (94.8 g, 966 mmol) and dimethylacetamide (DMA) (1 L) were introduced to a 2 L round bottom flask, and the mixture was stirred for 24 hours at a temperature of 150° C.

After the reaction was completed, the result was filtered under reduced pressure using dichloromethane (DCM) to remove the salt, and the solvent was removed using a rotary evaporator for concentration.

After that, the reaction material was separated by column chromatography (diethyl ether:hexane=1:10 (volume ratio)) to obtain Intermediate C-2 (143 g, 444 mmol, yield 92%).

### Preparation Example 3-2. Preparation of Intermediate C-1

Intermediate C-2 (143 g, 444 mmol), N-bromosuccinimide (NBS) (158 g, 888 mmol) and dimethylformide (DMF) (1.4 L) were introduced to a 2 L round bottom flask, and the mixture was stirred for 24 hours.

After the reaction was completed, the result was concentrated and separated by column chromatography (diethyl ether:hexane=1:5 (volume ratio)) to obtain Intermediate C-1 (109 g, 271 mmol, yield 61%).

### Preparation Example 3-3. Preparation of Intermediate C

Intermediate C-1 (109 g, 271 mmol), KOPiv (76 g, 542 mmol), dimethylacetamide (DMA) (1 L) and [1,4-bis(diphenylphosphino)butane] (η³-allyl)palladium(II) chloride (PdCl(C₃H₅) (dppb)) (8.26 g, 13.6 mmol) were introduced to a 2 L round bottom flask in consecutive order, and the mixture was stirred for 24 hours at a temperature of 150° C.

After the reaction was completed, the result was filtered under reduced pressure using dichloromethane (DCM) to remove the salt, and the solvent was removed using a rotary evaporator for concentration.

After that, the reaction material was separated by column chromatography (diethyl ether:hexane=2:3 (volume ratio)) to obtain Intermediate C (19.9 g, 62.3 mmol, yield 23%).

### Preparation Example 4. Preparation of Intermediate D

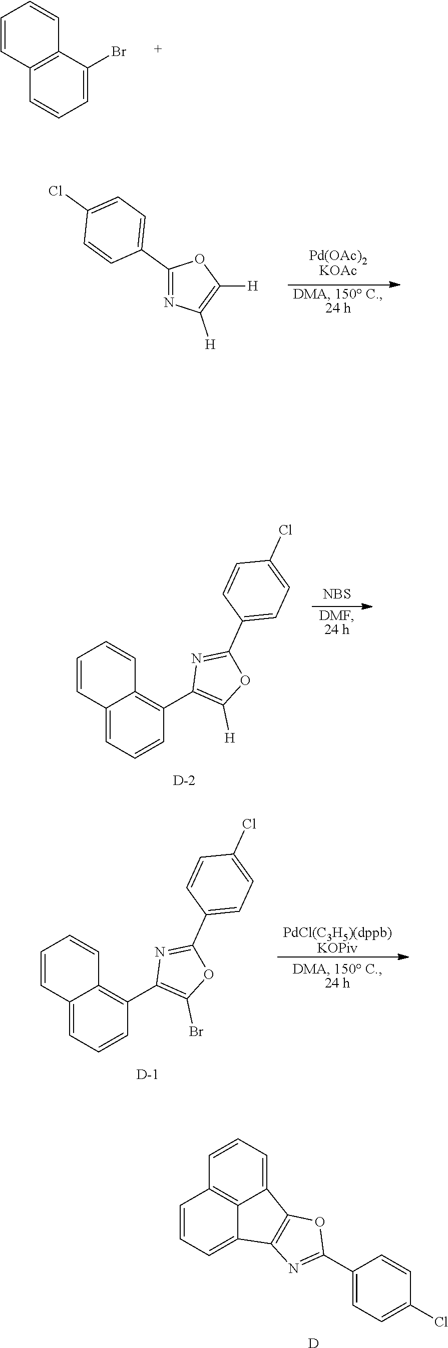

### Preparation Example 4-1. Preparation of Intermediate D-2

1-bromonaphthalene (100 g, 483 mmol), 2-(4-chlorophenyl)oxazole (130 g, 724 mmol), palladium acetate (Pd(OAc)₂) (5.42 g, 24.2 mmol), potassium acetate (KOAc) (94.8 g, 966 mmol) and dimethylacetamide (DMA) (1 L) were introduced to a 2 L round bottom flask, and the mixture was stirred for 24 hours at a temperature of 150° C.

After the reaction was completed, the result was filtered under reduced pressure using dichloromethane (DCM) to remove the salt, and the solvent was removed using a rotary evaporator for concentration.

After that, the reaction material was separated by column chromatography (diethyl ether:hexane=1:10 (volume ratio)) to obtain Intermediate D-2 (130 g, 425 mmol, yield 88%).

### Preparation Example 4-2. Preparation of Intermediate D-1

Intermediate D-2 (130 g, 425 mmol), N-bromosuccinimide (NBS) (151 g, 850 mmol) and dimethylformide (DMF) (1.3 L) were introduced to a 2 L round bottom flask, and the mixture was stirred for 24 hours.

After the reaction was completed, the result was concentrated and separated by column chromatography (diethyl ether:hexane=1:5 (volume ratio)) to obtain Intermediate D-1 (105 g, 272 mmol, yield 64%).

### Preparation Example 4-3. Preparation of Intermediate D

Intermediate D-1 (105 g, 272 mmol), KOPiv (76.3 g, 544 mmol), dimethylacetamide (DMA) (1 L) and [1,4-bis(diphenylphosphino)butane] (η³-allyl)palladium(II) chloride (PdCl(C₃H₅) (dppb)) (8.26 g, 13.6 mmol) were introduced to a 2 L round bottom flask in consecutive order, and the mixture was stirred for 24 hours at a temperature of 150° C.

After the reaction was completed, the result was filtered under reduced pressure using dichloromethane (DCM) to remove the salt, and the solvent was removed using a rotary evaporator for concentration.

After that, the reaction material was separated by column chromatography (diethyl ether:hexane=2:3 (volume ratio)) to obtain Intermediate D (17.4 g, 57.1 mmol, yield 21%).

### Preparation Example 5. Preparation of Compound 2

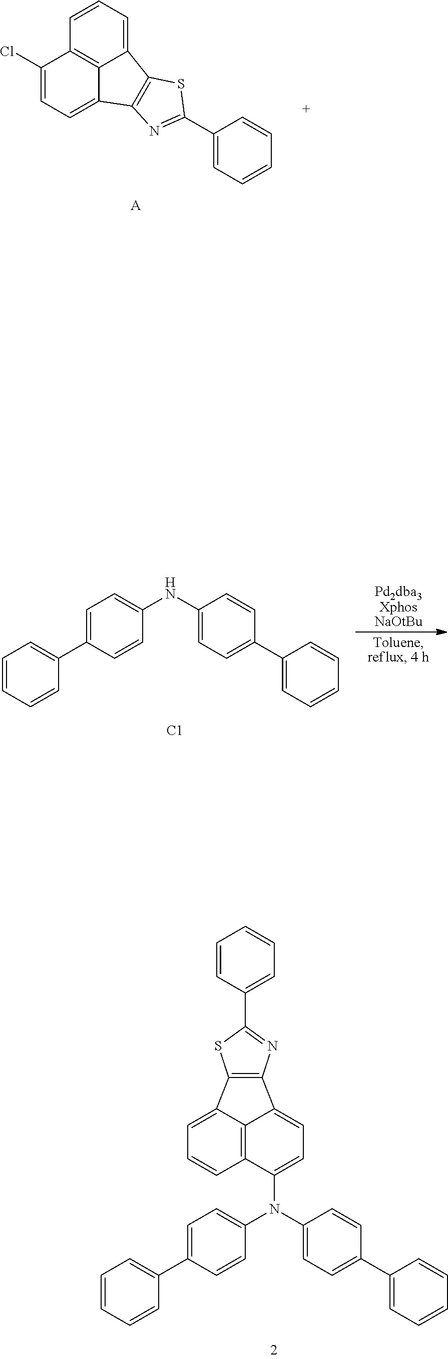

Intermediate A (7 g, 21.9 mmol), Compound C1 (7.04 g, 21.9 mmol), tris(dibenzylideneacetone)dipalladium(0) (Pd₂dba₃) (1 g, 1.1 mmol), dicyclohexyl(2′,4′,6′-triisopropyl-[1,1′-biphenyl]-2-yl)phosphine (Xphos) (4.91 g, 10.3 mmol), sodium tert-butoxide (NaOtBu) (4.21 g, 43.8 mmol) and toluene (70 mL) were introduced to a 250 mL round bottom flask, and the mixture was refluxed for 4 hours.

After the reaction was completed, the result was filtered under reduced pressure using dichloromethane (DCM) to remove the salt, and the solvent was removed using a rotary evaporator for concentration.

After that, the reaction material was separated by column chromatography (methylene chloride:hexane=1:3 (volume ratio)) to obtain Compound 2 (11.4 g, 18.8 mmol, yield 86%).

Target compounds were prepared as in the following Table 1 in the same manner as in Preparation Example 5 except that, in Preparation of Compound 2, Intermediate of the following Table 1 was used instead of Intermediate A, and Compound C of the following Table 1 was used instead of Compound C1.

TABLE 1

| Intermediate | Compound C | Target Compound | Yield |
|---|---|---|---|
| 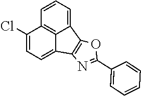 [SMILES: Clc1ccc2c3c(cccc13)-c1oc(-c3ccccc3)nc1-2] Intermediate B | 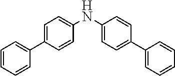 C1 | 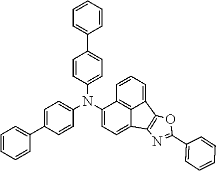 [SMILES: c1ccc(-c2ccc(N(c3ccc(-c4ccccc4)cc3)c3ccc4c5c(cccc35)-c3oc(-c5ccccc5)nc3-4)cc2)cc1] | 83% |
| | | 4 | |
| 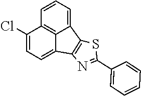 [SMILES: Clc1ccc2c3c(cccc13)-c1sc(-c3ccccc3)nc1-2] Intermediate A | 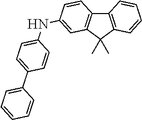 C2 | 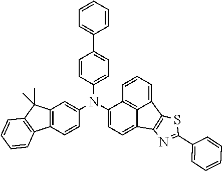 [SMILES: CC1(C)c2ccccc2-c2ccc(N(c3ccc(-c4ccccc4)cc3)c3ccc4c5c(cccc35)-c3sc(-c5ccccc5)nc3-4)cc21] | 87% |
| | | 26 | |
| 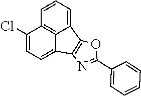 [SMILES: Clc1ccc2c3c(cccc13)-c1oc(-c3ccccc3)nc1-2] Intermediate B | 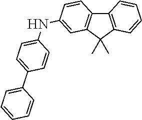 C2 | 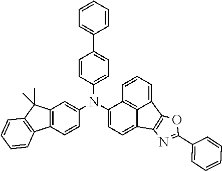 [SMILES: CC1(C)c2ccccc2-c2ccc(N(c3ccc(-c4ccccc4)cc3)c3ccc4c5c(cccc35)-c3oc(-c5ccccc5)nc3-4)cc21] | 84% |
| | | 28 | |
| 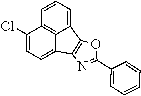 [SMILES: Clc1ccc2c3c(cccc13)-c1oc(-c3ccccc3)nc1-2] Intermediate B | 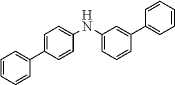 C4 | 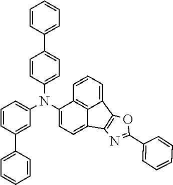 [SMILES: c1ccc(-c2ccc(N(c3cccc(-c4ccccc4)c3)c3ccc4c5c(cccc35)-c3oc(-c5ccccc5)nc3-4)cc2)cc1] | 88% |
| | | 104 | |
| 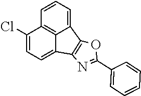 [SMILES: Clc1ccc2c3c(cccc13)-c1oc(-c3ccccc3)nc1-2] Intermediate B | 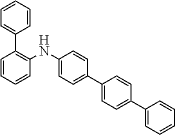 C6 | 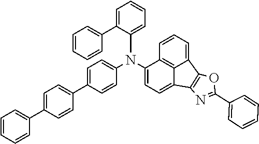 [SMILES: c1ccc(-c2ccc(-c3ccc(N(c4ccccc4-c4ccccc4)c4ccc5c6c(cccc46)-c4oc(-c6ccccc6)nc4-5)cc3)cc2)cc1] 176 | 84% |
| 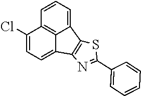 [SMILES: Clc1ccc2c3c(cccc13)-c1sc(-c3ccccc3)nc1-2] Intermediate A | 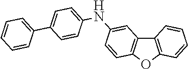 C9 | 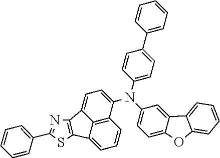 [SMILES: c1ccc(-c2ccc(N(c3ccc4oc5ccccc5c4c3)c3ccc4c5c(cccc35)-c3sc(-c5ccccc5)nc3-4)cc2)cc1] | 86% |
| | | 302 | |
| 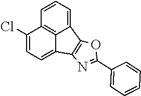 [SMILES: Clc1ccc2c3c(cccc13)-c1oc(-c3ccccc3)nc1-2] Intermediate B | 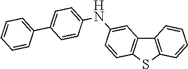 C10 | 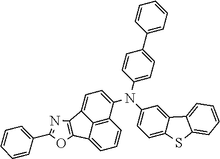 [SMILES: c1ccc(-c2ccc(N(c3ccc4sc5ccccc5c4c3)c3ccc4c5c(cccc35)-c3oc(-c5ccccc5)nc3-4)cc2)cc1] | 89% |
| | | 378 | |
| 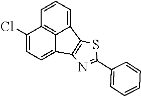 [SMILES: Clc1ccc2c3c(cccc13)-c1sc(-c3ccccc3)nc1-2] Intermediate A | 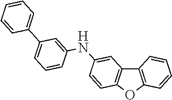 C11 | 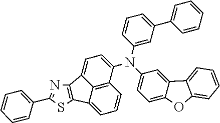 [SMILES: c1ccc(-c2cccc(N(c3ccc4oc5ccccc5c4c3)c3ccc4c5c(cccc35)-c3sc(-c5ccccc5)nc3-4)c2)cc1] | 82% |
| | | 402 | |
| 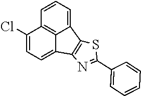 [SMILES: Clc1ccc2c3c(cccc13)-c1sc(-c3ccccc3)nc1-2] Intermediate A | 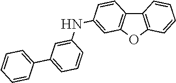 C12 | 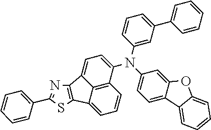 [SMILES: c1ccc(-c2cccc(N(c3ccc4c(c3)oc3ccccc34)c3ccc4c5c(cccc35)-c3sc(-c5ccccc5)nc3-4)c2)cc1] | 84% |
| | | 403 | |
| 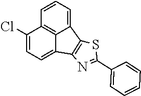 [SMILES: Clc1ccc2c3c(cccc13)-c1sc(-c3ccccc3)nc1-2] Intermediate A | 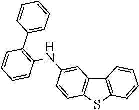 C14 | 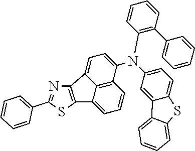 [SMILES: c1ccc(-c2nc3c(s2)-c2cccc4c(N(c5ccc6sc7ccccc7c6c5)c5ccccc5-c5ccccc5)ccc-3c24)cc1] | 79% |
| | | 430 | |
| 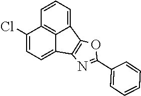 [SMILES: Clc1ccc2c3c(cccc13)-c1oc(-c3ccccc3)nc1-2] Intermediate B | 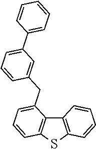 | 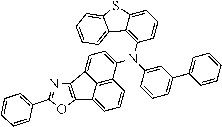 [SMILES: c1ccc(-c2cccc(N(c3ccc4c5c(cccc35)-c3oc(-c5ccccc5)nc3-4)c3cccc4sc5ccccc5c34)c2)cc1] 477 | 81% |
| | C16 | | |
| 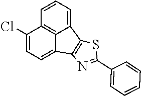 [SMILES: Clc1ccc2c3c(cccc13)-c1sc(-c3ccccc3)nc1-2] Intermediate A | 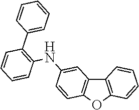 C13 | 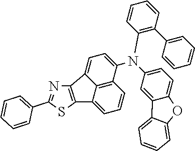 [SMILES: c1ccc(-c2nc3c(s2)-c2cccc4c(N(c5ccc6oc7ccccc7c6c5)c5ccccc5-c5ccccc5)ccc-3c24)cc1] | 78% |
| | | 498 | |
| 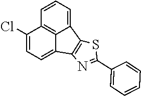 [SMILES: Clc1ccc2c3c(cccc13)-c1sc(-c3ccccc3)nc1-2] Intermediate A | 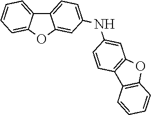 C17 | 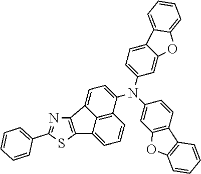 [SMILES: c1ccc(-c2nc3c(s2)-c2cccc4c(N(c5ccc6c(c5)oc5ccccc56)c5ccc6c(c5)oc5ccccc56)ccc-3c24)cc1] | 84% |
| | | 502 | |
| 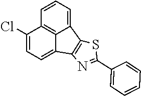 [SMILES: Clc1ccc2c3c(cccc13)-c1sc(-c3ccccc3)nc1-2] Intermediate A | 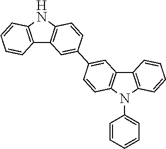 | 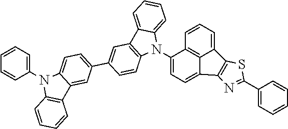 [SMILES: c1ccc(-c2nc3c(s2)-c2cccc4c(-n5c6ccccc6c6cc(-c7ccc8c(c7)c7ccccc7n8-c7ccccc7)ccc65)ccc-3c24)cc1] 530 | 79% |
| | C29 | | |
| 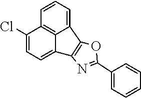 [SMILES: Clc1ccc2c3c(cccc13)-c1oc(-c3ccccc3)nc1-2] Intermediate B | 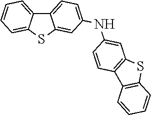 C18 | 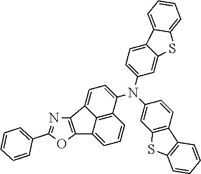 [SMILES: c1ccc(-c2nc3c(o2)-c2cccc4c(N(c5ccc6c(c5)sc5ccccc56)c5ccc6c(c5)sc5ccccc56)ccc-3c24)cc1] | 83% |
| | | 540 | |
| 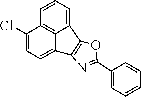 [SMILES: Clc1ccc2c3c(cccc13)-c1oc(-c3ccccc3)nc1-2] Intermediate B | 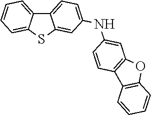 C19 | 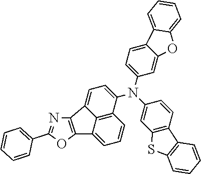 [SMILES: c1ccc(-c2nc3c(o2)-c2cccc4c(N(c5ccc6c(c5)oc5ccccc56)c5ccc6c(c5)sc5ccccc56)ccc-3c24)cc1] | 87% |
| | | 562 | |
| 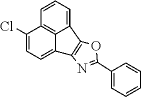 [SMILES: Clc1ccc2c3c(cccc13)-c1oc(-c3ccccc3)nc1-2] Intermediate B | 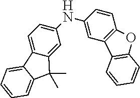 C20 | 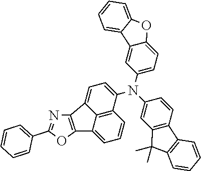 [SMILES: CC1(C)c2ccccc2-c2ccc(N(c3ccc4oc5ccccc5c4c3)c3ccc4c5c(cccc35)-c3oc(-c5ccccc5)nc3-4)cc21] | 81% |
| | | 569 | |
| 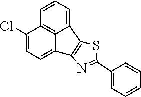 [SMILES: Clc1ccc2c3c(cccc13)-c1sc(-c3ccccc3)nc1-2] Intermediate A | 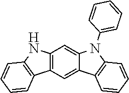 C28 | 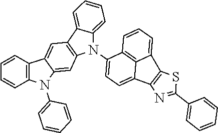 [SMILES: c1ccc(-c2nc3c(s2)-c2cccc4c(-n5c6ccccc6c6cc7c8ccccc8n(-c8ccccc8)c7cc65)ccc-3c24)cc1] | 74% |
| | | 577 | |
| 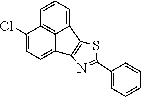 [SMILES: Clc1ccc2c3c(cccc13)-c1sc(-c3ccccc3)nc1-2] Intermediate A | 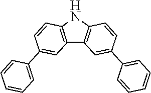 C27 | 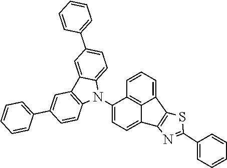 [SMILES: c1ccc(-c2ccc3c(c2)c2cc(-c4ccccc4)ccc2n3-c2ccc3c4c(cccc24)-c2sc(-c4ccccc4)nc2-3)cc1] | 78% |
| | | 702 | |
| 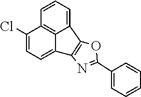 [SMILES: Clc1ccc2c3c(cccc13)-c1oc(-c3ccccc3)nc1-2] Intermediate B | 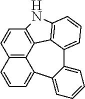 C31 | 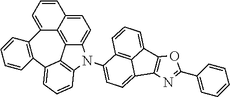 [SMILES: c1ccc(-c2nc3c(o2)-c2cccc4c(-n5c6cccc7c6c6c8c(cccc8ccc65)-c5ccccc5-7)ccc-3c24)cc1] 724 | 75% |

### Preparation Example 6. Preparation of Compound 20

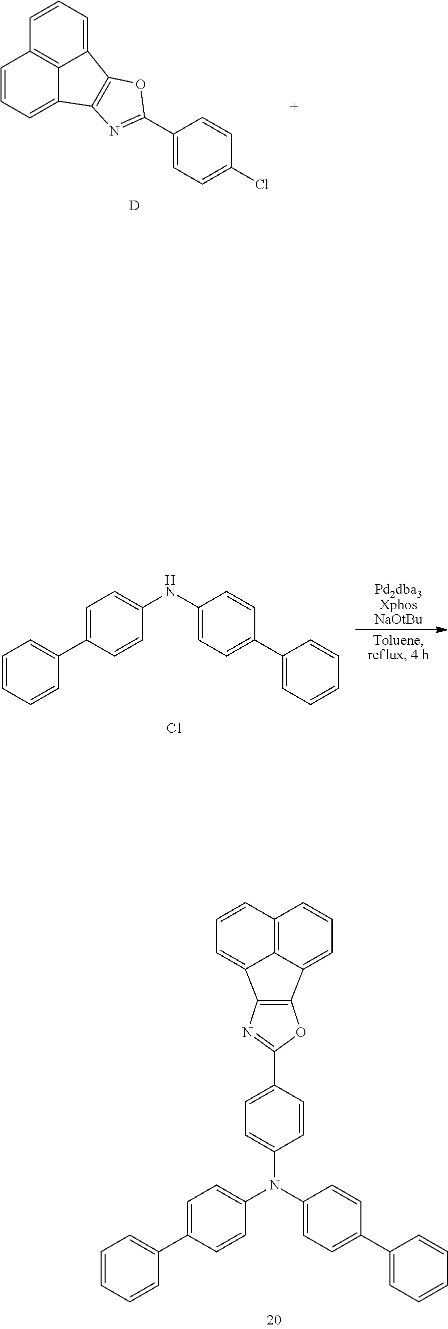

Intermediate D (7 g, 21.9 mmol), Compound C1 (7.04 g, 21.9 mmol), tris(dibenzylideneacetone)dipalladium(0) (Pd₂dba₃) (1 g, 1.1 mmol), dicyclohexyl(2′,4′,6′-triisopropyl-[1,1′-biphenyl]-2-yl)phosphine (Xphos) (4.91 g, 10.3 mmol), sodium tert-butoxide (NaOtBu) (4.21 g, 43.8 mmol) and toluene (70 mL) were introduced to a 250 mL round bottom flask, and the mixture was refluxed for 4 hours.

After the reaction was completed, the result was filtered under reduced pressure using dichloromethane (DCM) to remove the salt, and the solvent was removed using a rotary evaporator for concentration.

After that, the reaction material was separated by column chromatography (methylene chloride:hexane=1:3 (volume ratio)) to obtain Compound 20 (10.7 g, 18.2 mmol, yield 83%).

Target compounds were prepared as in the following Table 2 in the same manner as in Preparation Example 6 except that, in Preparation of Compound 20, Intermediate of the following Table 2 was used instead of Intermediate D, and Compound C of the following Table 2 was used instead of Compound C1.

TABLE 2

| Intermediate | Compound C | Target Compound | Yield |
|---|---|---|---|
| 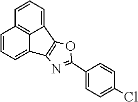 [SMILES: Clc1ccc(-c2nc3c(o2)-c2cccc4cccc-3c24)cc1] Intermediate D | 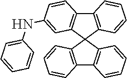 C3 | 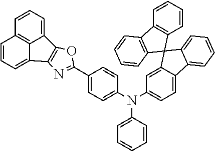 [SMILES: c1ccc(N(c2ccc(-c3nc4c(o3)-c3cccc5cccc-4c35)cc2)c2ccc3c(c2)C2(c4ccccc4-c4ccccc42)c2ccccc2-3)cc1] | 80% |
| | | 79 | |
| 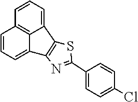 [SMILES: Clc1ccc(-c2nc3c(s2)-c2cccc4cccc-3c24)cc1] Intermediate C | 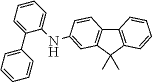 C5 | 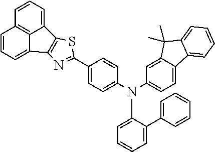 [SMILES: CC1(C)c2ccccc2-c2ccc(N(c3ccc(-c4nc5c(s4)-c4cccc6cccc-5c46)cc3)c3ccccc3-c3ccccc3)cc21] | 77% |
| | | 142 | |
| 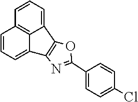 [SMILES: Clc1ccc(-c2nc3c(o2)-c2cccc4cccc-3c24)cc1] Intermediate D | 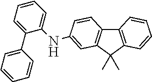 C5 | 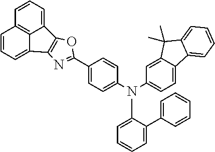 [SMILES: CC1(C)c2ccccc2-c2ccc(N(c3ccc(-c4nc5c(o4)-c4cccc6cccc-5c46)cc3)c3ccccc3-c3ccccc3)cc21] | 79% |
| | | 144 | |
| 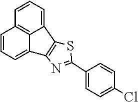 [SMILES: Clc1ccc(-c2nc3c(s2)-c2cccc4cccc-3c24)cc1] Intermediate C | 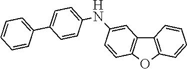 C9 | 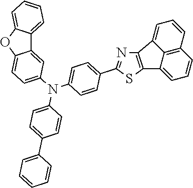 [SMILES: c1ccc(-c2ccc(N(c3ccc(-c4nc5c(s4)-c4cccc6cccc-5c46)cc3)c3ccc4oc5ccccc5c4c3)cc2)cc1] | 86% |
| | | 318 | |
| 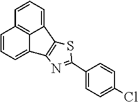 [SMILES: Clc1ccc(-c2nc3c(s2)-c2cccc4cccc-3c24)cc1] Intermediate C | 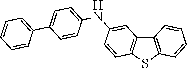 C10 | 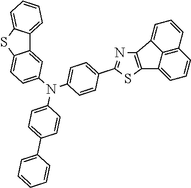 [SMILES: c1ccc(-c2ccc(N(c3ccc(-c4nc5c(s4)-c4cccc6cccc-5c46)cc3)c3ccc4sc5ccccc5c4c3)cc2)cc1] | 84% |
| | | 346 | |
| 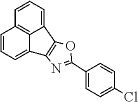 [SMILES: Clc1ccc(-c2nc3c(o2)-c2cccc4cccc-3c24)cc1] Intermediate D | 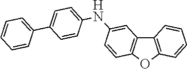 C9 | 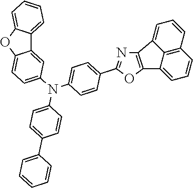 [SMILES: c1ccc(-c2ccc(N(c3ccc(-c4nc5c(o4)-c4cccc6cccc-5c46)cc3)c3ccc4oc5ccccc5c4c3)cc2)cc1] | 87% |
| | | 374 | |
| 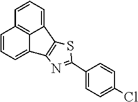 [SMILES: Clc1ccc(-c2nc3c(s2)-c2cccc4cccc-3c24)cc1] Intermediate C | 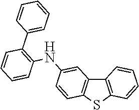 C14 | 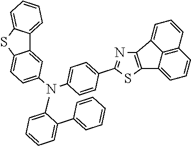 [SMILES: c1ccc(-c2ccccc2N(c2ccc(-c3nc4c(s3)-c3cccc5cccc-4c35)cc2)c2ccc3sc4ccccc4c3c2)cc1] | 81% |
| | | 446 | |
| 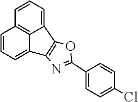 [SMILES: Clc1ccc(-c2nc3c(o2)-c2cccc4cccc-3c24)cc1] Intermediate D | 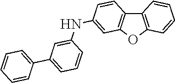 C12 | 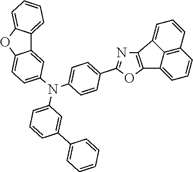 [SMILES: c1ccc(-c2cccc(N(c3ccc(-c4nc5c(o4)-c4cccc6cccc-5c46)cc3)c3ccc4oc5ccccc5c4c3)c2)cc1] | 84% |
| | | 474 | |
| 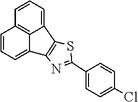 [SMILES: Clc1ccc(-c2nc3c(s2)-c2cccc4cccc-3c24)cc1] Intermediate C | 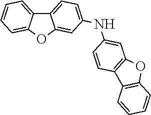 C17 | 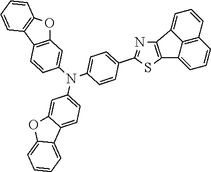 [SMILES: c1cc2c3c(cccc3c1)-c1sc(-c3ccc(N(c4ccc5c(c4)oc4ccccc45)c4ccc5c(c4)oc4ccccc45)cc3)nc1-2] | 82% |
| | | 518 | |
| 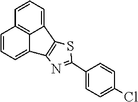 [SMILES: Clc1ccc(-c2nc3c(s2)-c2cccc4cccc-3c24)cc1] Intermediate C | 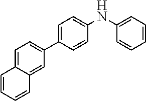 C26 | 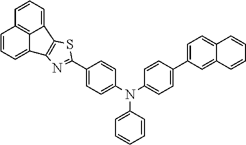 [SMILES: c1ccc(N(c2ccc(-c3ccc4ccccc4c3)cc2)c2ccc(-c3nc4c(s3)-c3cccc5cccc-4c35)cc2)cc1] | 79% |
| | | 645 | |

### Preparation Example 7. Preparation of Compound 602

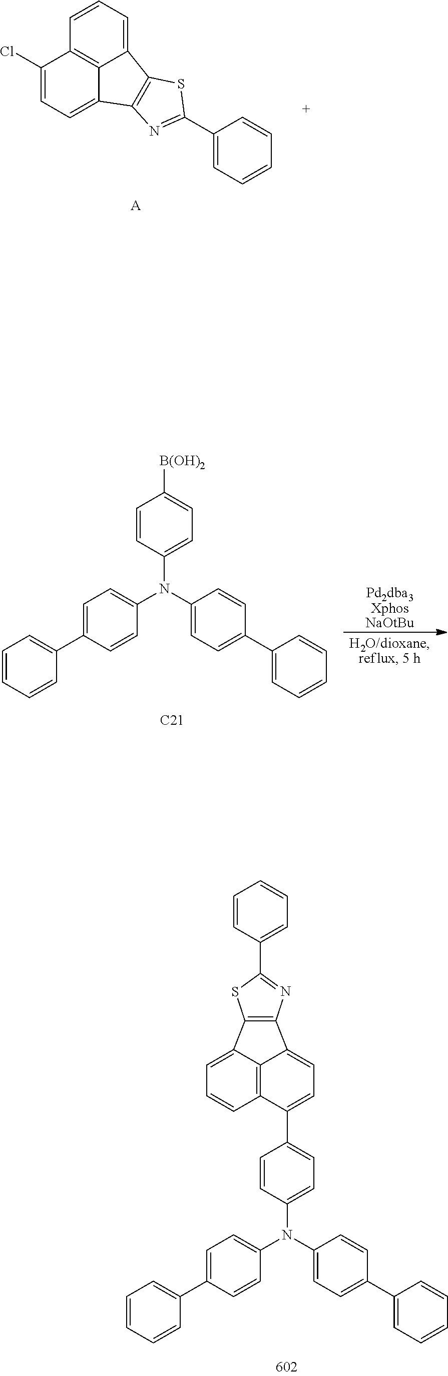

Intermediate A (7 g, 21.9 mmol), Compound C21 (10.1 g, 23 mmol), tris(dibenzylideneacetone)dipalladium(0) (Pd₂dba₃) (1 g, 1.1 mmol), dicyclohexyl(2′,4′,6′-triisopropyl-[1,1′-biphenyl]-2-yl)phosphine (Xphos) (1.04 g, 2.19 mmol), sodium tert-butoxide (NaOtBu) (4.21 g, 43.8 mmol) and toluene (70 mL) were introduced to a 250 mL round bottom flask, and the mixture was refluxed for hours.

After the reaction was completed, the result was filtered under reduced pressure using dichloromethane (DCM) to remove the salt, and the solvent was removed using a rotary evaporator for concentration.

After that, the reaction material was separated by column chromatography (methylene chloride:hexane=1:3 (volume ratio)) to obtain Compound 602 (11.3 g, 16.6 mmol, yield 76%).

Target compounds were prepared as in the following Table 3 in the same manner as in Preparation Example 7 except that, in Preparation of Compound 602, Intermediate of the following Table 3 was used instead of Intermediate A, and Compound C of the following Table 3 was used instead of Compound C21.

TABLE 3

| Intermediate | Compound C | Target Compound | Yield |
|---|---|---|---|
| 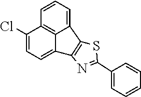 [SMILES: Clc1ccc2c3c(cccc13)-c1sc(-c3ccccc3)nc1-2] Intermediate A | 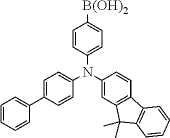 C22 | 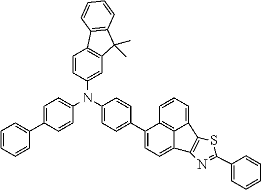 [SMILES: CC1(C)c2ccccc2-c2ccc(N(c3ccc(-c4ccccc4)cc3)c3ccc(-c4ccc5c6c(cccc46)-c4sc(-c6ccccc6)nc4-5)cc3)cc21] | 74% |
| | | 606 | |
| 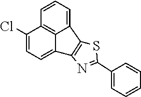 [SMILES: Clc1ccc2c3c(cccc13)-c1sc(-c3ccccc3)nc1-2] Intermediate A | 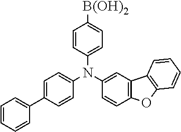 C23 | 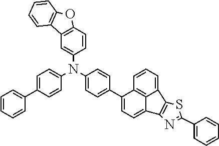 [SMILES: c1ccc(-c2ccc(N(c3ccc(-c4ccc5c6c(cccc46)-c4sc(-c6ccccc6)nc4-5)cc3)c3ccc4oc5ccccc5c4c3)cc2)cc1] | 80% |
| | | 610 | |
| 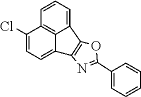 [SMILES: Clc1ccc2c3c(cccc13)-c1oc(-c3ccccc3)nc1-2] Intermediate B | 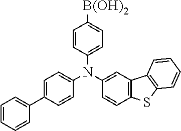 C24 | 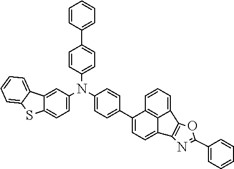 [SMILES: c1ccc(-c2ccc(N(c3ccc(-c4ccc5c6c(cccc46)-c4oc(-c6ccccc6)nc4-5)cc3)c3ccc4sc5ccccc5c4c3)cc2)cc1] | 82% |
| | | 616 | |
| 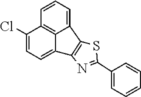 [SMILES: Clc1ccc2c3c(cccc13)-c1sc(-c3ccccc3)nc1-2] Intermediate A | 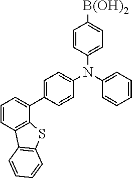 | 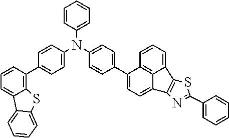 [SMILES: c1ccc(-c2nc3c(s2)-c2cccc4c(-c5ccc(N(c6ccccc6)c6ccc(-c7cccc8c7sc7ccccc78)cc6)cc5)ccc-3c24)cc1] 625 | 86% |
| | C25 | | |
| 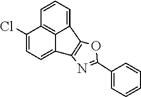 [SMILES: Clc1ccc2c3c(cccc13)-c1oc(-c3ccccc3)nc1-2] Intermediate B | 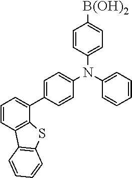 | 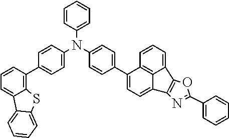 [SMILES: c1ccc(-c2nc3c(o2)-c2cccc4c(-c5ccc(N(c6ccccc6)c6ccc(-c7cccc8c7sc7ccccc78)cc6)cc5)ccc-3c24)cc1] 627 | 85% |
| | C25 | | |

Synthesis results for the compounds described in Preparation Examples 5 to 7 and Table 1 to Table 3 are shown in the following Table 4 and Table 5.

The following Table 4 shows measurement values of ¹H NMR (CDCl₃, 400 MHz), and the following Table 5 shows measurement values of ED-mass spectrometry (FD-MS: field desorption mass spectrometry).

TABLE 4

| Compound | ¹H NMR (CDCl₃, 400 MHz) |
|---|---|
| 2 | δ = 8.10~8.08 (d, 1H), 8.05~8.03 (d, 1H), 7.84~7.82 (d, |
| | 1H), 7.80~7.78 (d, 1H), 7.71~7.69 (d, 1H), 7.65~7.63 |
| | (d, 1H), 7.58~7.41 (m, 12H), 7.35~7.27 (m, 8H), |
| | 7.20~7.18 (d, 1H), 7.08~7.06 (t, 1H) |
| 4 | δ = 8.12~8.10 (d, 1H), 8.09~8.07 (d, 1H), 7.83~7.81 (d, |
| | 1H), 7.79~7.77 (d, 1H), 7.70~7.68 (d, 1H), 7.63~7.61 |
| | (d, 1H), 7.59~7.34(m, 13H), 7.32~7.20 (m, 8H), |
| | 7.14~7.12 (t, 1H) |
| 20 | δ = 8.21~8.19 (d, 1H), 8.17~8.15 (d, 1H), 7.99~7.97 (d, |
| | 2H), 7.86~7.84(d, 1H), 7.83~7.81 (d, 1H), 7.72~7.70 |
| | (d, 1H), 7.65~7.48 (m, 12H), 7.41~7.29 (m, 9H) |
| 26 | δ = 8.09~8.07 (d, 1H), 8.05~8.03 (d, 1H), 7.85~7.83 (d, |
| | 1H), 7.81~7.79 (d, 1H), 7.73~7.71 (d, 1H), 7.66~7.64 |
| | (d, 1H), 7.55~7.42(m, 11H), 7.33~7.17 (m, 8H), |
| | 7.08~7.06 (t, 1H), 1.30 (s, 6H) |
| 28 | δ = 8.11~8.09 (d, 1H), 8.08~8.06 (d, 1H), 7.81~7.79 (d, |
| | 1H), 7.77~7.75 (d, 1H), 7.69~7.67 (d, 1H), 7.64~7.62 |
| | (d, 1H), 7.57~7.35(m, 12H), 7.32~7.22 (m, 7H), |
| | 7.13~7.11 (t, 1H), 1.29 (s, 6H) |
| 79 | δ = 8.23~8.21 (d, 1H), 8.19~8.17 (d, 1H), 8.01~7.99 (d, |
| | 2H), 7.94~7.92 (d, 1H), 7.86~7.84(d, 1H), 7.72~7.70 |
| | (d, 1H), 7.68~7.65 (t, 1H), 7.56~7.54 (d, 2H), |
| | 7.49~7.47 (t, 2H), 7.38~7.33 (m, 5H), 7.25~7.00(m, |
| | 11H), 6.98~6.96 (d, 1H), 6.54~6.52 (d, 1H) |
| 104 | δ = 8.13~8.11 (d, 1H), 8.08~8.06 (d, 1H), 7.84~7.83 (d, |
| | 1H), 7.81~7.79 (d, 1H), 7.72~7.70 (d, 1H), 7.64~7.63 |
| | (d, 1H), 7.60 (s, 1H), 7.58~7.35(m, 12H), 7.32~7.22 |
| | (m, 8H), 7.12~7.10 (t, 1H) |
| 142 | δ = 8.20~8.18 (d, 1H), 8.15~8.13 (d, 1H), 7.98~7.96 (d, |
| | 2H), 7.87~7.85 (d, 1H), 7.83~7.81 (d, 1H), |
| | 7.57~7.41(m, 11H), 7.35~7.27 (m, 8H), 7.02~7.00 (t, |
| | 1H), 1.30 (s, 6H) |
| 144 | δ = 8.23~8.21 (d, 1H), 8.17~8.15 (d, 1H), 7.96~7.94 (d, |
| | 2H), 7.85~7.83 (d, 1H), 7.81~7.79 (d, 1H), |
| | 7.58~7.43(m, 11H), 7.37~7.29 (m, 8H), 7.01~6.99 (t, |
| | 1H), 1.32 (s, 6H) |
| 176 | δ = 8.15~8.13 (d, 1H), 8.08~8.06 (d, 1H), 7.81~7.79 (d, |
| | 1H), 7.75~7.73 (d, 1H), 7.69~7.67 (d, 1H), 7.63~7.61 |
| | (d, 2H), 7.57~7.35(m, 12H), 7.32~7.23 (m, 8H), |
| | 7.18~7.16 (t, 1H), 7.13~7.11 (d, 2H), 6.99~6.97 (d, |
| | 2H) |
| 302 | δ = 8.48 (s, 1H), 8.14~8.12 (d, 1H), 8.03~8.01 (d, 1H), |
| | 7.88~7.86 (d, 1H), 7.81~7.79 (d, 1H), 7.72~7.70 (d, |
| | 1H), 7.61~7.59 (d, 1H), 7.55~7.38(m, 10H), 7.35~7.29 |
| | (m, 7H), 7.22~7.20 (t, 1H), 7.02~7.00 (d, 1H) |
| 318 | δ = 8.45 (s, 1H), 8.19~8.17 (d, 1H), 8.10~8.08 (d, 1H), |
| | 7.97~7.95 (d, 2H), 7.86~7.84(d, 1H), 7.83~7.81 (d, |
| | 1H), 7.74~7.72 (d, 1H), 7.65~7.48 (m, 10H), 7.41~7.29 |
| | (m, 7H), 7.00~6.98 (d, 1H) |
| 346 | δ = 8.51~8.49 (d, 1H), 8.25 (s, 1H), 8.15~8.13 (d, 1H), |
| | 8.08~8.06 (d, 1H), 7.94~7.92 (d, 2H), 7.85~7.83(d, |
| | 1H), 7.80~7.78 (d, 1H), 7.72~7.70 (d, 1H), 7.62~7.45 |
| | (m, 10H), 7.43~7.28 (m, 7H), 7.24~7.22 (d, 1H) |
| 374 | δ = 8.43~8.41 (d, 1H), 8.27~8.25 (d, 1H), 7.97~7.95 (d, |
| | 2H), 7.88~7.86(d, 1H), 7.83~7.81 (d, 1H), 7.75~7.73 |
| | (d, 1H), 7.66~7.47 (m, 10H), 7.43~7.30 (m, 8H), |
| | 7.04~7.02 (d, 1H) |
| 378 | δ = 8.67 (s, 1H), 8.36~8.34 (d, 1H), 8.31~8.29 (d, 1H), |
| | 7.95~7.93 (d, 1H), 7.88~7.86 (d, 1H), 7.75~7.73 (d, |
| | 2H), 7.66~7.64 (d, 1H), 7.53~7.38 (m, 10H), 7.36~7.28 |
| | (m, 7H), 7.24~7.22 (d, 1H) |
| 402 | δ = 8.42 (s, 1H), 8.23~8.21 (d, 1H), 8.17~8.15 (d, 1H), |
| | 8.03~8.01 (d, 1H), 7.89~7.85 (d, 2H), 7.77~7.75 (d, |
| | 1H), 7.62~7.60 (d, 1H), 7.56~7.38(m, 9H), 7.36 (s, |
| | 1H), 7.33~7.29 (m, 5H), 7.24~7.22 (d, 2H), 6.99~6.97 |
| | (d, 1H) |
| 403 | δ = 8.23~8.21 (m, 2H), 8.16~8.14 (d, 1H), 8.03~8.01 (d, |
| | 1H), 7.88~7.86 (d, 2H), 7.75~7.73 (d, 1H), 7.62~7.60 |
| | (d, 1H), 7.59~7.38(m, 9H), 7.34 (s, 1H), 7.32~7.26 |
| | (m, 5H), 7.23~7.21 (d, 2H), 6.99~6.97 (d, 1H) |
| 430 | δ = 8.64~8.62 (d, 1H), 8.32~8.30 (d, 1H), 8.26 (s, 1H), |
| | 8.21~8.19 (d, 1H), 8.17~8.15 (d, 1H), 8.02~8.00 (d, |
| | 1H), 7.88~7.86 (d, 2H), 7.79~7.77 (d, 1H), 7.74~7.72 |
| | (d, 1H), 7.59~7.40 (m, 8H), 7.33~7.29 (m, 5H), |
| | 7.11~7.09 (d, 2H), 6.99~6.97 (t, 1H) |
| 446 | δ = 8.61~8.59 (d, 1H), 8.33~8.31 (d, 1H), 8.23~8.21 (d, |
| | 2H), 8.02~8.00 (d, 1H), 7.94~7.92 (d, 1H), 7.86~7.84 |
| | (d, 1H), 7.74~7.72 (d, 1H), 7.65~7.47 (m, 9H), |
| | 7.43~7.29 (m, 7H), 7.13~7.11 (d, 1H), 6.98~6.96 (t, |
| | 1H) |
| 474 | δ = 8.43 (s, 1H), 8.30~8.28 (d, 1H), 8.21~8.19 (d, 1H), |
| | 8.15~8.13 (d, 1H), 8.01~7.99 (d, 1H), 7.92~7.90 (d, |
| | 1H), 7.83~7.81 (d, 1H), 7.73~7.71 (d, 1H), 7.64~7.46 |
| | (m, 9H), 7.42~7.30 (m, 7H), 7.23~7.21 (d, 1H), |
| | 7.16~7.14 (d, 1H) |
| 477 | δ = 8.61~8.59 (d, 1H), 8.32~8.30 (d, 1H), 8.26~8.24 (d, |
| | 1H), 8.16~8.14 (d, 1H), 8.02~8.00 (m, 2H), 7.82~7.80 |
| | (d, 1H), 7.65~7.63 (d, 1H), 7.60~7.59 (d, 1H), |
| | 7.56~7.42(m, 9H), 7.39 (s, 1H), 7.35~7.28 (m, 6H), |
| | 7.24~7.22 (d, 1H) |
| 498 | δ = 8.43 (s, 1H), 8.28~8.26 (d, 1H), 8.24~8.21 (m, 3H), |
| | 8.12~8.10 (d, 1H), 8.08~8.06 (d, 1H), 8.02~8.00 (d, |
| | 1H), 7.88~7.86 (d, 1H), 7.80~7.78 (d, 1H), 7.63~7.61 |
| | (d, 1H), 7.59~7.42 (m, 7H), 7.33~7.29 (m, 5H), |
| | 7.12~7.10 (m, 2H), 6.98~6.96 (t, 1H) |
| 502 | δ = 8.27 (s, 1H), 8.24 (s, 1H), 8.17~8.15 (d, 1H), |
| | 8.11~8.09 (d, 1H), 8.02~8.00 (d, 1H), 7.94~7.92 (d, |
| | 1H), 7.89~7.87 (d, 1H), 7.77~7.75 (d, 1H), 7.72~7.70 |
| | (d, 1H), 7.61~7.59 (d, 1H), 7.56~7.39 (m, 6H), |
| | 7.34~7.26 (m, 6H), 7.11~7.09 (t, 1H), 7.06~7.04 (d, |
| | 1H) |
| 518 | δ = 8.26 (s, 1H), 8.20~8.18 (d, 1H), 8.10~8.08 (d, 2H), |
| | 8.05~8.03 (d, 1H), 8.01~7.99 (d, 1H), 7.84~7.82 (d, |
| | 2H), 7.78~7.76 (d, 2H), 7.61~7.45 (m, 6H), 7.43~7.28 |
| | (m, 7H), 7.11~7.06 (d, 1H) |
| 530 | δ = 8.49~8.47 (d, 2H), 8.41~8.39 (d, 1H), 8.28~8.24 (m, |
| | 3H), 8.18~8.16 (d, 1H), 8.14~8.12 (d, 1H), 7.95~7.93 |
| | (d, 1H), 7.82~7.80 (d, 2H), 7.73~7.69 (m, 3H), |
| | 7.65~7.62 (m, 4H), 7.60~7.44 (m, 9H), 7.37~7.25 (m, |
| | 2H) |
| 540 | δ = 8.65~8.63 (d, 2H), 8.39~8.37 (d, 1H), 8.33~8.31 (d, |
| | 1H), 8.16~8.14 (d, 1H), 8.11~8.09 (d, 1H), 7.89~7.87 |
| | (d, 1H), 7.79~7.77 (d, 2H), 7.71~7.69 (d, 1H), |
| | 7.65~7.63 (d, 1H), 7.61~7.59 (d, 1H), 7.51~7.39(m, |
| | 5H), 7.35~7.27 (m, 6H), 7.24~7.22 (d, 1H) |
| 562 | δ = 8.63~8.61 (d, 1H), 8.31~8.29 (d, 1H), 8.25~8.23 (d, |
| | 1H), 8.12~8.10 (d, 1H), 8.04~8.02 (d, 1H), 7.81~7.79 |
| | (d, 1H), 7.73~7.71 (d, 1H), 7.65~7.63 (d, 1H), |
| | 7.57~7.38 (m, 8H), 7.35~7.29 (m, 6H), 7.23~7.21 (t, |
| | 1H), 7.04~7.02 (d, 1H) |
| 569 | δ = 8.16~8.14 (d, 1H), 8.09~8.07 (d, 1H), 7.83~7.81 (d, |
| | 1H), 7.78~7.76 (d, 1H), 7.65~7.63 (d, 1H), 7.60~7.58 |
| | (d, 1H), 7.55~7.34 (m, 11H), 7.32~7.23 (m, 6H), |
| | 7.15~7.13 (t, 1H), 1.30 (s, 6H) |
| 589 | δ = 8.74 (s, 1H), 8.65~8.63 (d, 1H), 8.35~8.33 (d, 1H), |
| | 8.31~8.29 (d, 1H), 8.17~8.15 (d, 1H), 8.12~8.10 (d, |
| | 1H), 7.86~7.84 (d, 1H), 7.71~7.53 (m, 6H), 7.53~31 |
| | (m, 10H), 7.00~7.98 (d, 1H), 7.98 (s, 1H) |
| 602 | δ = 8.13~8.11 (d, 1H), 8.01~7.99 (d, 1H), 7.91~7.89 (d, |
| | 1H), 7.85~7.83 (d, 1H), 7.81~7.79 (d, 1H), 7.72~7.70 |
| | (d, 1H), 7.66~7.64 (d, 2H), 7.58~7.41 (m, 14H), |
| | 7.35~7.27 (m, 9H), 7.23~7.21 (d, 1H) |
| 604 | δ = 8.11~8.09 (d, 1H), 7.96~7.94 (d, 1H), 7.85~7.83 (d, |
| | 1H), 7.80~7.78 (d, 1H), 7.73~7.71 (d, 1H), 7.67~7.65 |
| | (d, 1H), 7.63~7.61 (d, 2H), 7.58~7.34 (m, 14H), |
| | 7.32~7.22 (m, 9H), 7.19~7.17 (d, 1H) |
| 606 | δ = 8.10~8.08 (d, 1H), 8.04~8.02 (d, 1H), 7.84~7.82 (d, |
| | 1H), 7.79~7.77 (d, 1H), 7.71~7.69 (d, 1H), 7.65~7.63 |
| | (d, 1H), 7.58~7.42 (m, 13H), 7.35~7.20 (m, 10H), |
| | 7.10~7.08 (t, 1H), 1.32 (s, 6H) |
| 610 | δ = 8.47 (s, 1H), 8.12~8.10 (d, 1H), 8.02~8.00 (d, 1H), |
| | 7.89~7.87 (d, 1H), 7.82~7.80 (d, 1H), 7.71~7.69 (d, |
| | 1H), 7.65~7.63 (d, 1H), 7.56~7.38 (m, 12H), 7.36~7.28 |
| | (m, 9H), 7.23~7.21 (t, 1H), 7.03~7.01 (d, 1H) |
| 616 | δ = 8.51 (s, 1H), 8.21~8.19 (d, 1H), 8.12~8.10 (d, 1H), |
| | 7.93~7.91 (d, 1H), 7.85~7.83 (d, 1H), 7.73~7.71 (d, |
| | 1H), 7.63~7.61 (d, 1H), 7.57~7.36 (m, 12H), 7.35~7.26 |
| | (m, 9H), 7.21~7.19 (t, 1H), 7.00~6.88 (d, 1H) |
| 625 | δ = 8.65~8.63 (d, 1H), 8.56~8.54 (d, 1H), 8.43~8.41 (d, |
| | 1H), 8.25~8.23 (d, 1H), 8.21~8.19 (d, 1H), 8.13~8.11 |
| | (d, 1H), 7.87~7.85 (d, 1H), 7.83~7.80 (m, 2H), |
| | 7.65~7.63 (d, 1H), 7.56~7.42(m, 9H), 7.35~7.28 (m, |
| | 7H), 7.24~7.22 (d, 1H), 7.10~7.08 (d, 1H), 7.06~7.04 |
| | (d, 1H), 6.87~6.85 (t, 1H) |
| 627 | δ = 8.62~8.60 (d, 1H), 8.54~8.52 (d, 1H), 8.41~8.39 (d, |
| | 1H), 8.22~8.20 (d, 1H), 8.17~8.15 (d, 1H), 8.11~8.09 |
| | (d, 1H), 7.84~7.82 (d, 1H), 7.80~7.78 (m, 2H), |
| | 7.64~7.62 (d, 1H), 7.54~7.41(m, 9H), 7.36~7.28 (m, |
| | 7H), 7.23~7.21 (d, 1H), 7.12~7.10 (d, 1H), 7.07~7.05 |
| | (d, 1H), 6.89~6.87 (t, 1H) |
| 645 | δ = 8.26~8.24 (m, 2H), 8.21~8.19 (d, 1H), 8.08~8.06 (d, |
| | 1H), 8.05~8.03 (d, 1H), 7.99~7.97 (d, 1H), 7.94~7.92 |
| | (d, 1H), 7.85~7.83 (d, 1H), 7.81~7.79 (d, 1H), |
| | 7.72~7.70 (d, 2H), 7.65~7.51 (m, 7H), 7.41~7.32 (m, |
| | 5H), 7.11~7.09 (d, 1H), 7.07~7.05 (d, 1H), 6.88~6.86 |
| | (t, 1H) |
| 702 | δ = 8.45 (s, 2H), 8.15~8.13 (d, 1H), 8.07~8.05 (d, 1H), |
| | 8.03~8.01 (d, 1H), 7.75~7.73 (d, 4H), 7.67~7.51 (m, |
| | 8H), 7.44~7.24 (m, 10H), |
| 724 | δ = 8.21~8.19 (d, 1H), 8.15~8.13 (d, 1H), 7.95 (d, 1H), |
| | 7.80~7.77 (m, 4H), 7.75~7.73 (d, 2H), 7.67~7.59 (m, |
| | 9H), 7.43~7.36 (m, 4H) |

TABLE 5

| Compound | FD-MS | Compound | FD-MS |
|---|---|---|---|
| 2 | m/z = 604.2042(C₄₃H₂₈N₂S, 604.1973) | 518 | m/z = 632.1549(C₄₃H₂₄N₂O₂S, 632.1558) |
| 4 | m/z = 588.2104(C₄₃H₂₈N₂O, 588.2202) | 530 | m/z = 691.2048(C₄₉H₂₉N₃S, 691.2082) |
| 20 | m/z = 588.1985(C₄₃H₂₈N₂O, 588.2202) | 474 | m/z = 602.2041(C₄₃H₂₆N₂O₂, 602.1994) |
| 26 | m/z = 644.2319(C₄₆H₃₂N₂S, 644.2286) | 477 | m/z = 618.1776(C₄₃H₂₆N₂Oₛ, 618.1766) |
| 28 | m/z = 628.2531(C₄₆H₃₂N₂O, 628.2515) | 498 | m/z = 618.1817(C₄₃H₂₆N₂OS, 618.1766) |
| 79 | m/z = 674.2381(C₅₀H₃₀N₂O, 674.2358) | 502 | m/z = 632.1625(C₄₃H₂₄N₂O₂S, 632.1558) |
| 104 | m/z = 588.2193(C₄₃H₂₈N₂O, 588.2202) | 518 | m/z = 632.1549(C₄₃H₂₄N₂O₂S, 632.1558) |
| 142 | m/z = 644.2309(C₄₆H₃₂N₂S, 644.2286) | 530 | m/z = 691.2048(C₄₉H₂₉N₃S, 691.2082) |
| 144 | m/z = 628.2531(C₄₆H₃₂N₂O, 628.2515) | 540 | m/z = 648.1352(C₄₃H₂₄N₂OS₂, 648.1330) |
| 176 | m/z = 664.2484(C₄₉H₃₂N₂O, 664.2515) | 562 | m/z = 632.1570(C₄₃H₂₄N₂O₂S, 632.1558) |
| 302 | m/z = 618.1824(C₄₃H₂₆N₂OS, 618.1766) | 569 | m/z = 642.2341(C₄₆H₃₀N₂O₂, 642.2307) |
| 318 | m/z = 618.1844(C₄₃H₂₆N₂OS, 618.1766) | 589 | m/z = 615.1802(C₄₃H₂₅N₃S, 615.1769) |
| 346 | m/z = 634.1580(C₄₃H₂₆N₂S₂, 634.1537) | 602 | m/z = 680.2318(C₄₉H₃₂N₂S, 680.2286) |
| 374 | m/z = 602.2172(C₄₃H₂₆H₂O₂, 602.1994) | 604 | m/z = 664.2395(C₄₉H₃₂N₂O, 664.2515) |
| 378 | m/z = 618.1829(C₄₃H₂₆N₂OS, 618.1766) | 606 | m/z = 720.2618(C₅₂H₃₆N₂S, 720.2599) |
| 402 | m/z = 618.1803(C₄₃H₂₆N₂OS, 618.1766) | 610 | m/z = 694.2107(C₄₉H₃₀N₂OS, 694.2079) |
| 403 | m/z = 618.1684(C₄₃H₂₆N₂OS, 618.1766) | 616 | m/z = 694.2087(C₄₉H₃₀N₂OS, 694.2079) |
| 430 | m/z = 634.1547(C₄₃H₂₆N₂S₂, 634.1537) | 625 | m/z = 710.1873(C₄₉H₃₀N₂S₂, 710.1850) |
| 446 | m/z = 634.1603(C₄₃H₂₆N₂S₂, 634.1537) | 627 | m/z = 694.2086(C₄₉H₃₀N₂OS, 694.2079) |
| 474 | m/z = 602.2041(C₄₃H₂₆N₂O₂, 602.1994) | 645 | m/z = 578.1884(C₄₁H₂₆N₂S, 578.1817) |
| 477 | m/z = 618.1776(C₄₃H₂₆N₂OS, 618.1766) | 702 | m/z = 602.1835(C₄₃H₂₆N₂S, 602.1817) |
| 498 | m/z = 618.1817(C₄₃H₂₆N₂OS, 618.1766) | 724 | m/z = 558.1760(C₄₁H₂₂N₂O, 558.1732) |
| 502 | m/z = 632.1625(C₄₃H₂₄N₂O₂S, 632.1558) | | |

### Experimental Example 1

### Experimental Example 1-1. Manufacture of Organic Light Emitting Device

A glass substrate coated with ITO as a thin film to a thickness of 1,500 Å was cleaned with distilled water ultrasonic waves. After the cleaning with distilled water was finished, the substrate was ultrasonic cleaned with solvents such as acetone, methanol and isopropyl alcohol, then dried, and then subjected to UVO (ultraviolet ozone) treatment for 5 minutes using UV (ultraviolet) in a UV cleaner. After that, the substrate was transferred to a plasma cleaner (PT), and after subjected to plasma treatment under vacuum for ITO work function and residual film removal, the substrate was transferred to a thermal deposition apparatus for organic deposition.

On the transparent ITO electrode (positive electrode), 4,4′,4″-tris[2-naphthyl(phenyl)amino]triphenylamine (2-TNATA), a hole injection layer, having a thickness of 600 Å and N,N′-bis(α-naphthyl)-N,N′-diphenyl-4,4′-diamine (NPB), a hole transport layer, having a thickness of 300 Å were deposited.

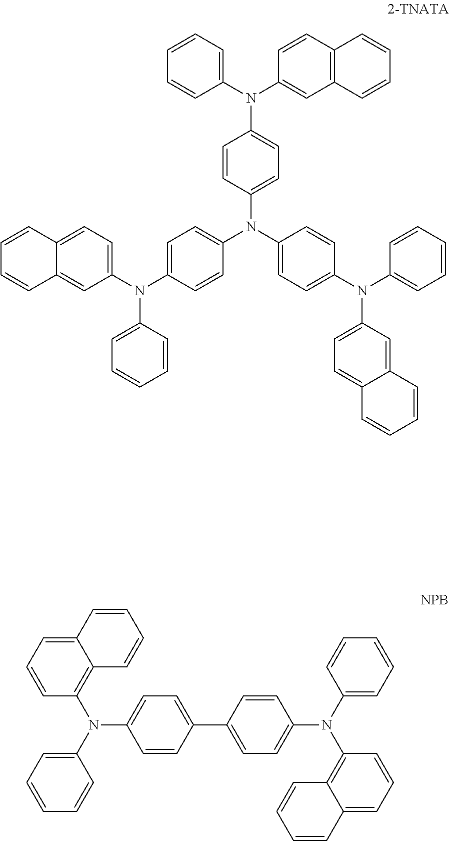 [SMILES: c1ccc(N(c2ccc(N(c3ccc(N(c4ccccc4)c4ccc5ccccc5c4)cc3)c3ccc(N(c4ccccc4)c4ccc5ccccc5c4)cc3)cc2)c2ccc3ccccc3c2)cc1 ; c1ccc(N(c2ccc(-c3ccc(N(c4ccccc4)c4cccc5ccccc45)cc3)cc2)c2cccc3ccccc23)cc1]

A light emitting layer was thermal vacuum deposited thereon as follows. The light emitting layer was deposited to a thickness of 500 Å by depositing a compound described in the following Table 6 as a red host (or green host), and, using (piq)₂(Ir) (acac) as a red phosphorescent dopant, doping the host with 3 wt % of the (piq)₂ (Ir) (acac).

After that, BCP was deposited to a thickness of 60 Å as a hole blocking layer, and Alq₃ was deposited to a thickness of 200 Å thereon as an electron transport layer. Lastly, an electron injection layer was formed on the electron transport layer by depositing lithium fluoride (LiF) to a thickness of 10 Å, and then a negative electrode was formed on the electron injection layer by depositing aluminum (Al) to a thickness of 1,200 Å, and as a result, an organic electroluminescent device was manufactured.

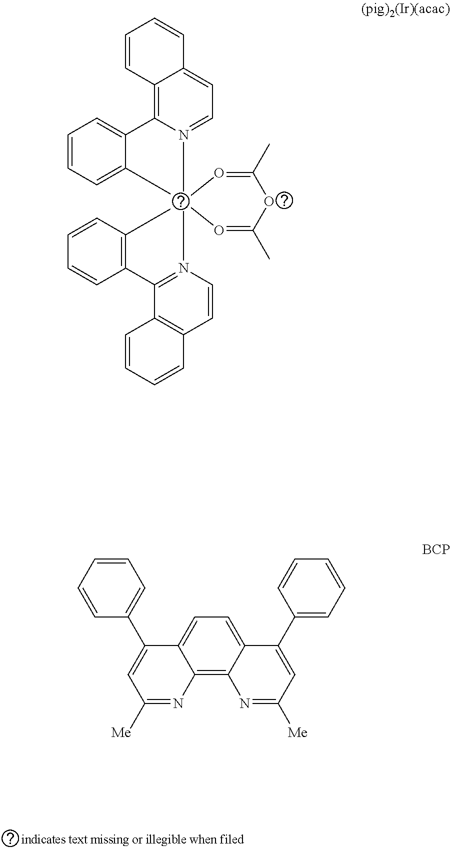

Meanwhile, all the organic compounds required to manufacture the OLED were vacuum sublimation purified under 10⁻⁸ torr to 10⁻⁶ torr for each material to be used in the OLED (organic light emitting device) manufacture.

### Experimental Example 1-2. Driving Voltage and Light Emission Efficiency of Organic Light Emitting Device

For each of the organic light emitting devices manufactured as above, electroluminescent (EL) properties were measured using M7000 manufactured by McScience Inc., and with the measurement results, T₉₀ was measured when standard luminance was 6,000 cd/m² through a lifetime measurement system (M6000) manufactured by McScience Inc. Results of measuring driving voltage, light emission efficiency, color coordinate (CIE) and lifetime of each of the organic light emitting devices manufactured according to the present disclosure are shown in the following Table 6.

T₉₀ means a lifetime (unit: hour), a time taken for luminance to become 90% with respect to initial luminance.

TABLE 6

| | | Driving | | Color | |
|---|---|---|---|---|---|
| | | Voltage | Efficiency | Coordinate | Lifetime |
| | Compound | (V) | (cd/A) | (x, y) | (T₉₀) |
| Comparative | A | 5.18 | 33.8 | (0.685, 0.315) | 30 |
| Example 1 | | | | | |
| Comparative | B | 4.97 | 36.1 | (0.685, 0.315) | 27 |
| Example 2 | | | | | |
| Comparative | C | 4.83 | 39.7 | (0.685, 0.315) | 41 |
| Example 3 | | | | | |
| Comparative | D | 5.94 | 42.6 | (0.685, 0.315) | 56 |
| Example 4 | | | | | |
| Example 1 | 2 | 4.53 | 46.8 | (0.685, 0.315) | 77 |
| Example 2 | 4 | 4.55 | 44.5 | (0.685, 0.315) | 78 |
| Example 3 | 20 | 4.51 | 54.6 | (0.685, 0.315) | 80 |
| Example 4 | 26 | 3.99 | 64.9 | (0.685, 0.315) | 131 |
| Example 5 | 28 | 4.64 | 63.2 | (0.685, 0.315) | 129 |
| Example 6 | 79 | 4.32 | 51.8 | (0.685, 0.315) | 135 |
| Example 7 | 104 | 4.23 | 56.4 | (0.685, 0.315) | 85 |
| Example 8 | 142 | 4.47 | 67.8 | (0.685, 0.315) | 144 |
| Example 9 | 144 | 4.49 | 62.4 | (0.685, 0.315) | 138 |
| Example 10 | 176 | 4.31 | 65.8 | (0.685, 0.315) | 124 |
| Example 11 | 302 | 4.20 | 64.6 | (0.685, 0.315) | 153 |
| Example 12 | 318 | 4.38 | 55.2 | (0.685, 0.315) | 148 |
| Example 13 | 346 | 4.47 | 56.4 | (0.685, 0.315) | 130 |
| Example 14 | 374 | 4.39 | 53.4 | (0.685, 0.315) | 126 |
| Example 15 | 378 | 4.37 | 58.6 | (0.685, 0.315) | 163 |
| Example 16 | 402 | 4.53 | 65.9 | (0.685, 0.315) | 155 |
| Example 17 | 403 | 4.50 | 63.8 | (0.685, 0.315) | 145 |
| Example 18 | 430 | 4.37 | 57.2 | (0.685, 0.315) | 135 |
| Example 19 | 446 | 4.45 | 56.0 | (0.685, 0.315) | 131 |
| Example 20 | 474 | 4.22 | 45.5 | (0.685, 0.315) | 94 |
| Example 21 | 477 | 4.43 | 45.8 | (0.685, 0.315) | 86 |
| Example 22 | 498 | 4.07 | 64.7 | (0.685, 0.315) | 145 |
| Example 23 | 502 | 4.18 | 54.6 | (0.685, 0.315) | 163 |
| Example 24 | 518 | 4.23 | 58.8 | (0.685, 0.315) | 146 |
| Example 25 | 530 | 4.36 | 61.6 | (0.685, 0.315) | 180 |
| Example 26 | 540 | 4.45 | 46.4 | (0.685, 0.315) | 82 |
| Example 27 | 562 | 4.49 | 47.8 | (0.685, 0.315) | 88 |
| Example 28 | 569 | 4.50 | 43.9 | (0.685, 0.315) | 165 |
| Example 29 | 589 | 4.48 | 61.5 | (0.685, 0.315) | 181 |
| Example 30 | 602 | 4.31 | 56.9 | (0.685, 0.315) | 133 |
| Example 31 | 604 | 4.32 | 57.2 | (0.685, 0.315) | 123 |
| Example 32 | 606 | 4.45 | 55.1 | (0.685, 0.315) | 195 |
| Example 33 | 610 | 4.43 | 54.3 | (0.685, 0.315) | 106 |
| Example 34 | 616 | 4.45 | 52.0 | (0.685, 0.315) | 98 |
| Example 35 | 625 | 4.59 | 59.8 | (0.685, 0.315) | 134 |
| Example 36 | 627 | 4.47 | 55.7 | (0.685, 0.315) | 118 |
| Example 37 | 645 | 4.51 | 47.5 | (0.685, 0.315) | 101 |
| Example 38 | 702 | 4.30 | 57.8 | (0.685, 0.315) | 138 |
| Example 39 | 724 | 4.43 | 58.1 | (0.685, 0.315) | 134 |

[Comparative Compounds]

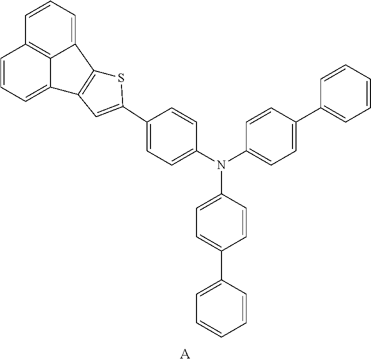

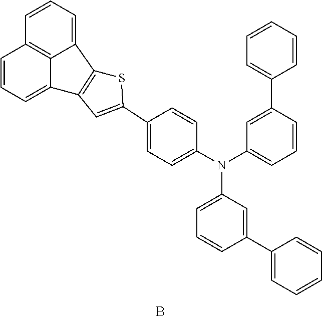

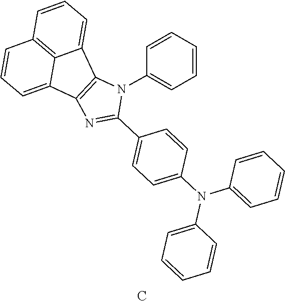

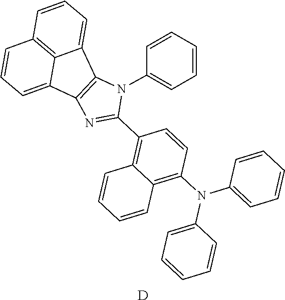

From the results of Table 6, it was identified that the organic light emitting devices of Examples 1 to 39 using the heterocyclic compound of Chemical Formula 1 of the present disclosure as a host of an organic material layer, particularly a light emitting layer, of the organic light emitting device had improved driving voltage, efficiency and lifetime compared to the organic light emitting devices of Comparative Examples 1 to 4.

Compounds A to D used in Comparative Examples 1 to 4 have low thermal stability, whereas the heterocyclic compound of Chemical Formula 1 of the present disclosure has proper molecular weight and bandgap while having high thermal stability. The proper bandgap of the light emitting layer has a favorable hole transport ability and prevents electron losses, which helps with effective formation of a recombination zone. Accordingly, as seen from the results of Table 6, it was identified that the Examples using the heterocyclic compound of the present disclosure had improved performance compared to the Comparative Examples.

In addition, it was identified that the heterocyclic compound represented by Chemical Formula 1 according to the present disclosure had a smaller T1 compared to Compounds A to D used in Comparative Examples 1 to 4. When a host material has a T1 value higher than a dopant material but lower than other host materials, excited electrons of the host material more readily migrate to the dopant material, and triplet-triplet Dexter energy transfer more readily occurs. In other words, Dexter energy transfer, an energy transfer mechanism for phosphorescence having maximum efficiency of 100%, more readily occurs than Forster resonance energy transfer, an energy transfer mechanism for fluorescence having maximum efficiency of 25%, and as a result, higher efficiency may be expected.

In addition, general bis-carbazole that has been used as a P-type host in the art was not effective in terms of energy transfer when used as a red host material, however, it was identified that the heterocyclic compound represented by Chemical Formula 1 according to the present disclosure used in Examples 1 to 39 was suitable for use as a red host by improving stability of excited electrons through effectively lowering T1 (triplet energy level), and by enhancing molecular stability.

### REFERENCE NUMERAL

100: Substrate
200: Positive Electrode
300: Organic Material Layer
301: Hole Injection Layer
302: Hole Transport Layer
303: Light Emitting Layer
304: Hole Blocking Layer
305: Electron Transport Layer
306: Electron Injection Layer
400: Negative Electrode

1. A heterocyclic compound represented by the following Chemical Formula 1: 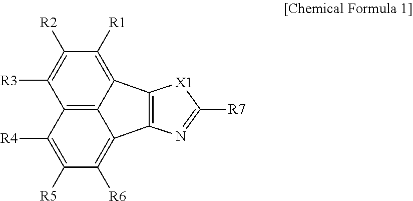
wherein, in Chemical Formula 1,
X1 is O; or S;
R1 to R6 are the same as or different from each other, and each independently selected from the group consisting of hydrogen; deuterium; halogen; a cyano group; a substituted or unsubstituted C1 to C60 alkyl group; a substituted or unsubstituted C2 to C60 alkenyl group; a substituted or unsubstituted C2 to C60 alkynyl group; a substituted or unsubstituted C1 to C60 alkoxy group; a substituted or unsubstituted C3 to C60 cycloalkyl group; a substituted or unsubstituted C2 to C60 heterocycloalkyl group; a substituted or unsubstituted C6 to C60 aryl group; a substituted or unsubstituted C2 to C60 heteroaryl group; —P(═O)R101R102; —SiR101R102R103; and a group represented by the following Chemical Formula 2, or two or more groups adjacent to each other bond to each other to form a substituted or unsubstituted C6 to C60 aromatic hydrocarbon ring or a substituted or unsubstituted C2 to C60 heteroring, wherein R101, R102 and R103 are the same as or different from each other and each independently a substituted or unsubstituted C1 to C60 alkyl group; a substituted or unsubstituted C6 to C60 aryl group; or a substituted or unsubstituted C2 to C60 heteroaryl group;
R7 is a substituted or unsubstituted C6 to C60 aryl group; or a group represented by the following Chemical Formula 2; and
at least one of R1 to R7 is the group represented by the following Chemical Formula 2, 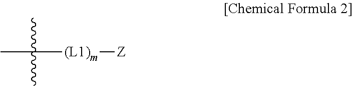
in Chemical Formula 2,
L1 is a direct bond; a substituted or unsubstituted C6 to C60 arylene group; or a substituted or unsubstituted C2 to C60 heteroarylene group;
m is an integer of 0 to 5, and when m is 2 or greater, L1s are the same as or different from each other; and
Z is selected from the group consisting of hydrogen; deuterium; halogen; a cyano group; a substituted or unsubstituted C1 to C60 alkyl group; a substituted or unsubstituted C2 to C60 alkenyl group; a substituted or unsubstituted C2 to C60 alkynyl group; a substituted or unsubstituted C1 to C60 alkoxy group; a substituted or unsubstituted C3 to C60 cycloalkyl group; a substituted or unsubstituted C2 to C60 heterocycloalkyl group; a substituted or unsubstituted C6 to C60 aryl group; a substituted or unsubstituted C2 to C60 heteroaryl group; a substituted or unsubstituted C6 to C60 monocyclic or polycyclic arylamine group; a substituted or unsubstituted C2 to C60 monocyclic or polycyclic heteroarylamine group; —P(═O)R101R102; and —SiR101R102R103, or two or more groups adjacent to each other bond to each other to form a substituted or unsubstituted C6 to C60 aromatic hydrocarbon ring or a substituted or unsubstituted C2 to C60 heteroring, wherein R101, R102 and R103 are the same as or different from each other and each independently a substituted or unsubstituted C1 to C60 alkyl group; a substituted or unsubstituted C6 to C60 aryl group; or a substituted or unsubstituted C2 to C60 heteroaryl group.

2. The heterocyclic compound of claim 1, wherein Chemical Formula 1 is represented by any one of the following Chemical Formulae 1-1 and 1-2: 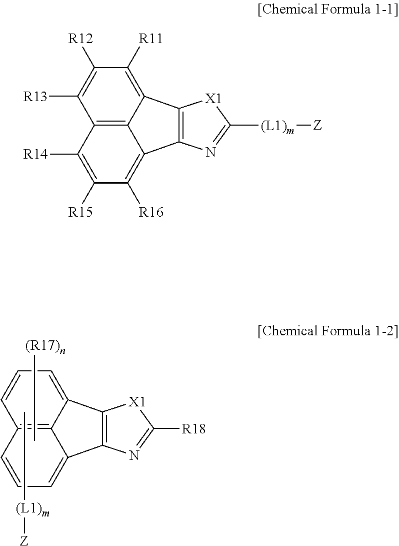
in Chemical Formulae 1-1 and 1-2,
R11 to R17 are the same as or different from each other and each independently selected from the group consisting of hydrogen; deuterium; halogen; a cyano group; a substituted or unsubstituted C1 to C60 alkyl group; a substituted or unsubstituted C2 to C60 alkenyl group; a substituted or unsubstituted C2 to C60 alkynyl group; a substituted or unsubstituted C1 to C60 alkoxy group; a substituted or unsubstituted C3 to C60 cycloalkyl group; a substituted or unsubstituted C2 to C60 heterocycloalkyl group; a substituted or unsubstituted C6 to C60 aryl group; a substituted or unsubstituted C2 to C60 heteroaryl group; —P(═O)R101R102; and —SiR101R102R103, wherein R101, R102 and R103 are the same as or different from each other and each independently a substituted or unsubstituted C1 to C60 alkyl group; a substituted or unsubstituted C6 to C60 aryl group; or a substituted or unsubstituted C2 to C60 heteroaryl group;
R18 is a substituted or unsubstituted C6 to C60 aryl group;
n is an integer of 0 to 5, and when n is 2 or greater, R17s are the same as or different from each other;
X1 has the same definition as in Chemical Formula 1; and
L1, m and Z have the same definitions as in Chemical Formula 2.

3. The heterocyclic compound of claim 2, wherein Chemical Formula 1-2 is represented by any one of the following Chemical Formulae 1-2-1 to 1-2-4: 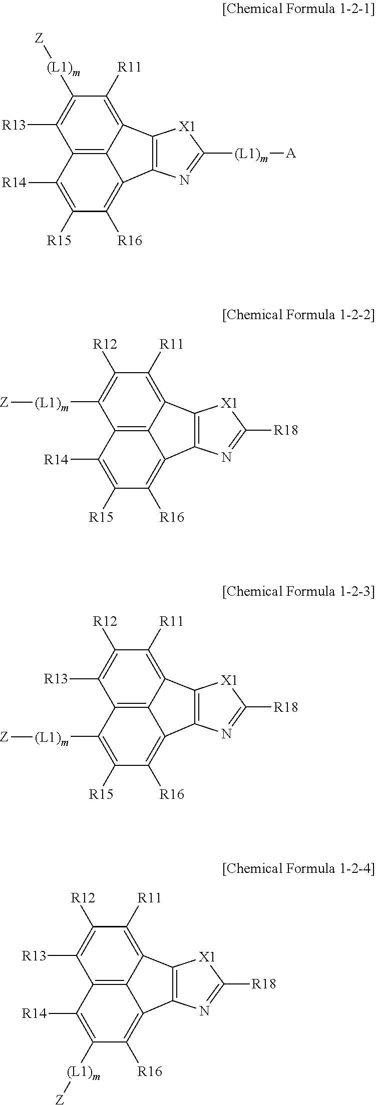
in Chemical Formulae 1-2-1 to 1-2-4,
X1 has the same definition as in Chemical Formula 1;
R11 to R16 have the same definitions as in Chemical Formula 1-1;
R18 has the same definition as in Chemical Formula 1-2; and
L1, m and Z have the same definitions as in Chemical Formula 2.

4. The heterocyclic compound of claim 1, wherein Chemical Formula 1 is represented by any one of the following Chemical Formulae 1-3-1 to 1-3-3: 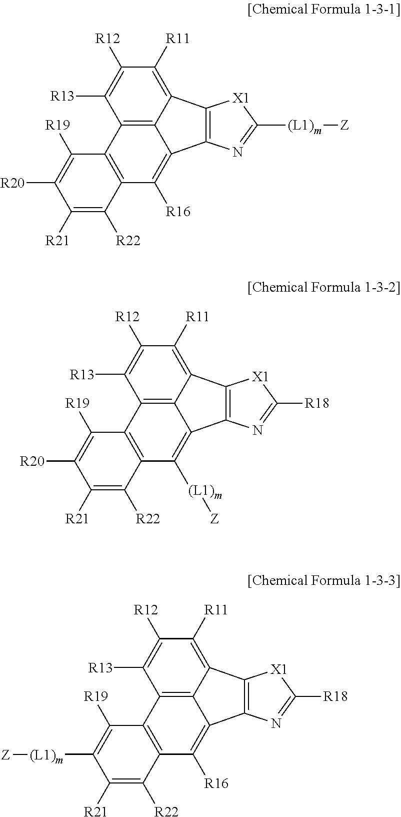
in Chemical Formulae 1-3-1 to 1-3-3,
R19 to R22 are the same as or different from each other and each independently selected from the group consisting of hydrogen; deuterium; halogen; a cyano group; a substituted or unsubstituted C1 to C60 alkyl group; a substituted or unsubstituted C2 to C60 alkenyl group; a substituted or unsubstituted C2 to C60 alkynyl group; a substituted or unsubstituted C1 to C60 alkoxy group; a substituted or unsubstituted C3 to C60 cycloalkyl group; a substituted or unsubstituted C2 to C60 heterocycloalkyl group; a substituted or unsubstituted C6 to C60 aryl group; a substituted or unsubstituted C2 to C60 heteroaryl group; —P(═O)R101R102; and —SiR101R102R103, wherein R101, R102 and R103 are the same as or different from each other and each independently a substituted or unsubstituted C1 to C60 alkyl group; a substituted or unsubstituted C6 to C60 aryl group; or a substituted or unsubstituted C2 to C60 heteroaryl group;
X1 has the same definition as in Chemical Formula 1;
R11 to R13 and R16 have the same definitions as in Chemical Formula 1-1;
R18 has the same definition as in Chemical Formula 1-2; and
L1, m and Z have the same definitions as in Chemical Formula 2.

5. The heterocyclic compound of claim 1, wherein Chemical Formula 1 is represented by any one of the following Chemical Formulae 1-4-1 and 1-4-2: 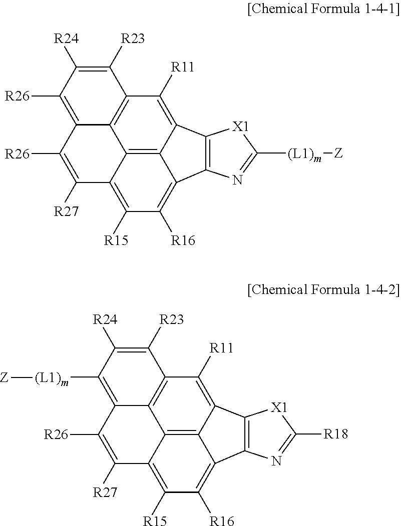
in Chemical Formulae 1-4-1 and 1-4-2,
R23 to R27 are the same as or different from each other and each independently selected from the group consisting of hydrogen; deuterium; halogen; a cyano group; a substituted or unsubstituted C1 to C60 alkyl group; a substituted or unsubstituted C2 to C60 alkenyl group; a substituted or unsubstituted C2 to C60 alkynyl group; a substituted or unsubstituted C1 to C60 alkoxy group; a substituted or unsubstituted C3 to C60 cycloalkyl group; a substituted or unsubstituted C2 to C60 heterocycloalkyl group; a substituted or unsubstituted C6 to C60 aryl group; a substituted or unsubstituted C2 to C60 heteroaryl group; —P(═O)R101R102; and —SiR101R102R103, wherein R101, R102 and R103 are the same as or different from each other and each independently a substituted or unsubstituted C1 to C60 alkyl group; a substituted or unsubstituted C6 to C60 aryl group; or a substituted or unsubstituted C2 to C60 heteroaryl group;
X1 has the same definition as in Chemical Formula 1;
R11, R15 and R16 have the same definitions as in Chemical Formula 1-1;
R18 has the same definition as in Chemical Formula 1-2; and
L1, m and Z have the same definitions as in Chemical Formula 2.

6. The heterocyclic compound of claim 1, wherein Z is a hole transporting group.

7. The heterocyclic compound of claim 1, wherein Z is represented by any one of the following Chemical Formulae 3-1 and 3-2: 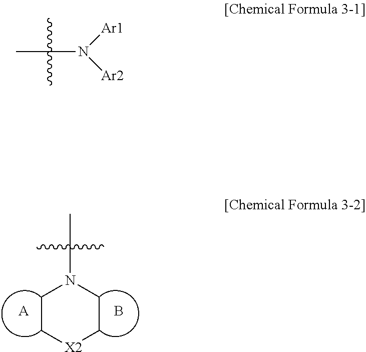
in Chemical Formulae 3-1 and 3-2,
Ar1 and Ar2 are the same as or different from each other, and each independently a substituted or unsubstituted C6 to C60 aryl group; or a substituted or unsubstituted C2 to C60 heteroaryl group;
X2 is a direct bond; 0; S; or NRa;
Ra is selected from the group consisting of hydrogen; deuterium; halogen; a cyano group; a substituted or unsubstituted C1 to C60 alkyl group; a substituted or unsubstituted C2 to C60 alkenyl group; a substituted or unsubstituted C2 to C60 alkynyl group; a substituted or unsubstituted C1 to C60 alkoxy group; a substituted or unsubstituted C3 to C60 cycloalkyl group; a substituted or unsubstituted C2 to C60 heterocycloalkyl group; a substituted or unsubstituted C6 to C60 aryl group; a substituted or unsubstituted C2 to C60 heteroaryl group; —P(═O)R101R102; and —SiR101R102R103, wherein R101, R102 and R103 are the same as or different from each other and each independently a substituted or unsubstituted C1 to C60 alkyl group; a substituted or unsubstituted C6 to C60 aryl group; or a substituted or unsubstituted C2 to C60 heteroaryl group; and
the A ring and the B ring are the same as or different from each other and each independently a substituted or unsubstituted C6 to C60 aryl ring; a substituted or unsubstituted C4 to C60 heteroaryl ring; a substituted or unsubstituted C5 to C60 cycloalkyl ring; or a substituted or unsubstituted C5 to C60 cycloalkenyl ring, two or more groups of substituents of the A ring and the B ring adjacent to each other bond to each other to form a substituted or unsubstituted C5 to C60 hydrocarbon ring or a substituted or unsubstituted C4 to C60 heteroring, and the hydrocarbon ring and the heteroring adjacent to each other bond to each other to form a fused ring.

8. The heterocyclic compound of claim 7, wherein Chemical Formula 3-2 is represented by any one of the following Chemical Formulae 3-2-1 to 3-2-7: 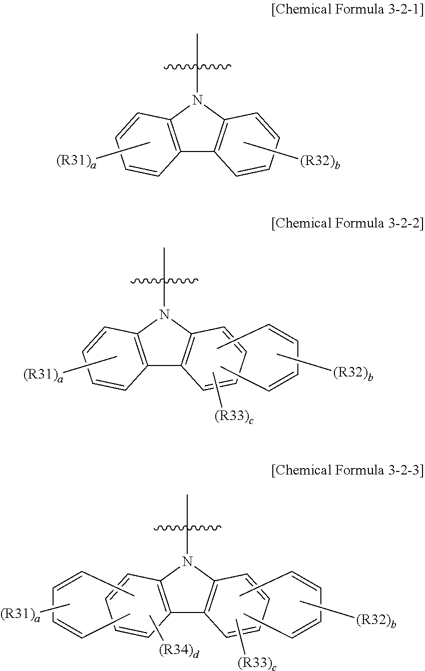 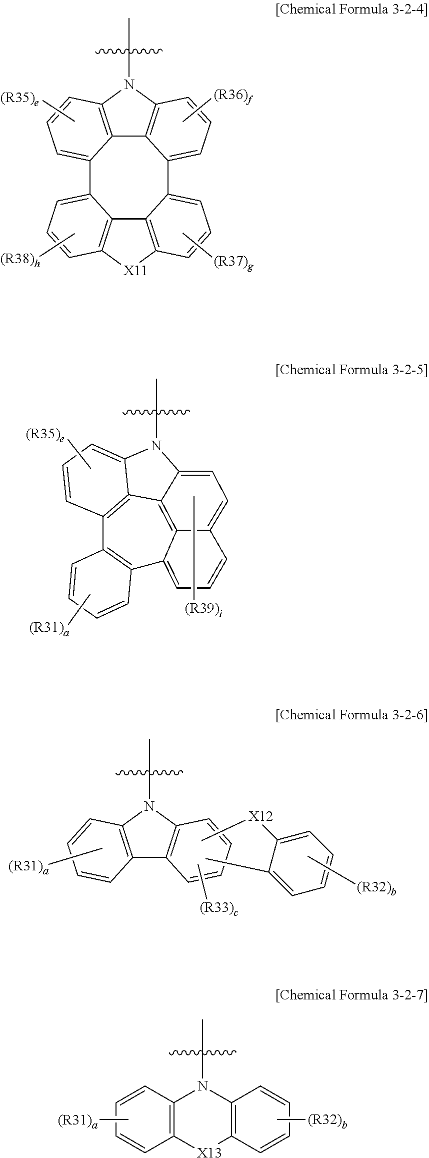
in Chemical Formulae 3-2-1 to 3-2-7,
X11 to X13 are the same as or different from each other, and each independently 0; S; NRb or CRcRd;
R31 to R39 and Rb to Rd are the same as or different from each other, and each independently selected from the group consisting of hydrogen; deuterium; halogen; a cyano group; a substituted or unsubstituted C1 to C60 alkyl group; a substituted or unsubstituted C2 to C60 alkenyl group; a substituted or unsubstituted C2 to C60 alkynyl group; a substituted or unsubstituted C1 to C60 alkoxy group; a substituted or unsubstituted C3 to C60 cycloalkyl group; a substituted or unsubstituted C2 to C60 heterocycloalkyl group; a substituted or unsubstituted C6 to C60 aryl group; a substituted or unsubstituted C2 to C60 heteroaryl group; —P(═O)R101R102; and —SiR101R102R103, or two or more groups adjacent to each other bond to each other to form a substituted or unsubstituted C6 to C60 aromatic hydrocarbon ring; or a substituted or unsubstituted C2 to C60 heteroring, wherein R101, R102 and R103 are the same as or different from each other and each independently a substituted or unsubstituted C1 to C60 alkyl group; a substituted or unsubstituted C6 to C60 aryl group; or a substituted or unsubstituted C2 to C60 heteroaryl group;
a and b are the same as or different from each other and each independently an integer of 0 to 4, and when a is 2 or greater, R3 is are the same as or different from each other, and when b is 2 or greater, R32s are the same as or different from each other;
c and d are the same as or different from each other and each independently an integer of 0 to 2, and when c is 2 or greater, R33s are the same as or different from each other, and when d is 2 or greater, R34s are the same as or different from each other;
e to h are the same as or different from each other and each independently an integer of 0 to 3, and when e is 2 or greater, R35s are the same as or different from each other, when f is 2 or greater, R36s are the same as or different from each other, when g is 2 or greater, R37s are the same as or different from each other, and when h is 2 or greater, R38s are the same as or different from each other; and
i is an integer of 0 to 5, and when i is 2 or greater, R39s are the same as or different from each other.

9. The heterocyclic compound of claim 1, wherein the heterocyclic compound represented by Chemical Formula 1 does not include deuterium as a substituent, or has a deuterium content of 1% to 100% with respect to a total number of hydrogen atoms and deuterium atoms.

10. The heterocyclic compound of claim 1, wherein Chemical Formula 1 is represented by any one of the following compounds:

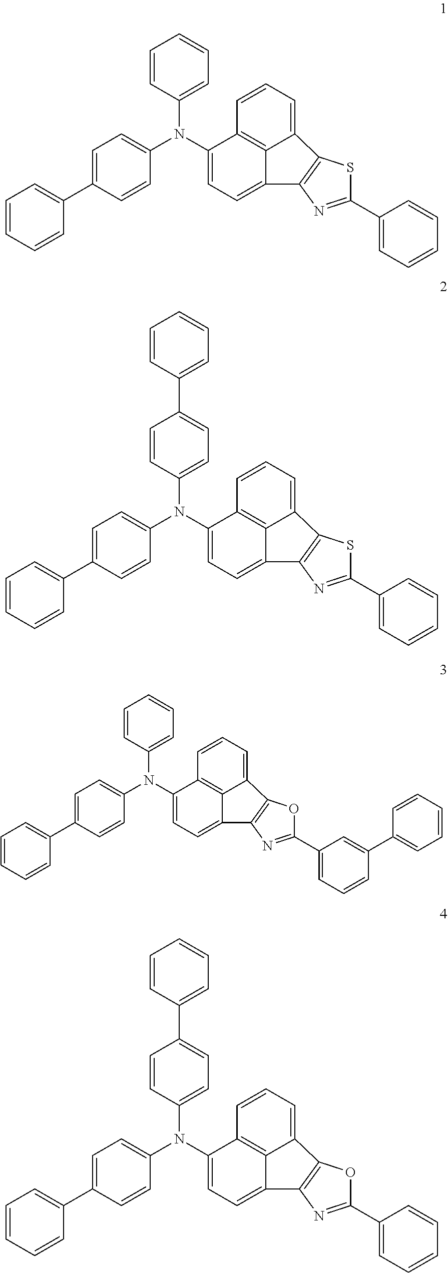 [SMILES: c1ccc(-c2ccc(N(c3ccccc3)c3ccc4c5c(cccc35)-c3sc(-c5ccccc5)nc3-4)cc2)cc1 ; c1ccc(-c2ccc(N(c3ccc(-c4ccccc4)cc3)c3ccc4c5c(cccc35)-c3sc(-c5ccccc5)nc3-4)cc2)cc1 ; c1ccc(-c2ccc(N(c3ccccc3)c3ccc4c5c(cccc35)-c3oc(-c5cccc(-c6ccccc6)c5)nc3-4)cc2)cc1 ; c1ccc(-c2ccc(N(c3ccc(-c4ccccc4)cc3)c3ccc4c5c(cccc35)-c3oc(-c5ccccc5)nc3-4)cc2)cc1]

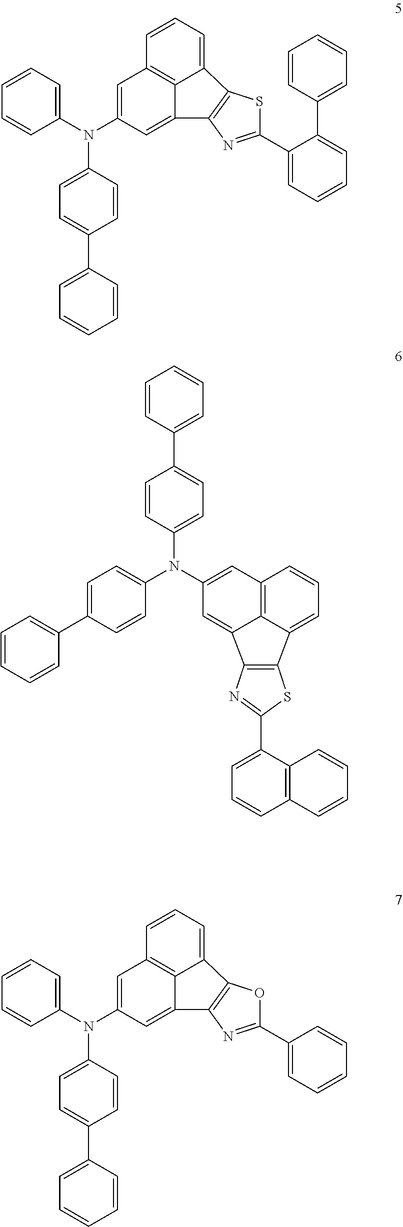 [SMILES: c1ccc(-c2ccc(N(c3ccccc3)c3cc4c5c(cccc5c3)-c3sc(-c5ccccc5-c5ccccc5)nc3-4)cc2)cc1 ; c1ccc(-c2ccc(N(c3ccc(-c4ccccc4)cc3)c3cc4c5c(cccc5c3)-c3sc(-c5cccc6ccccc56)nc3-4)cc2)cc1 ; c1ccc(-c2ccc(N(c3ccccc3)c3cc4c5c(cccc5c3)-c3oc(-c5ccccc5)nc3-4)cc2)cc1]

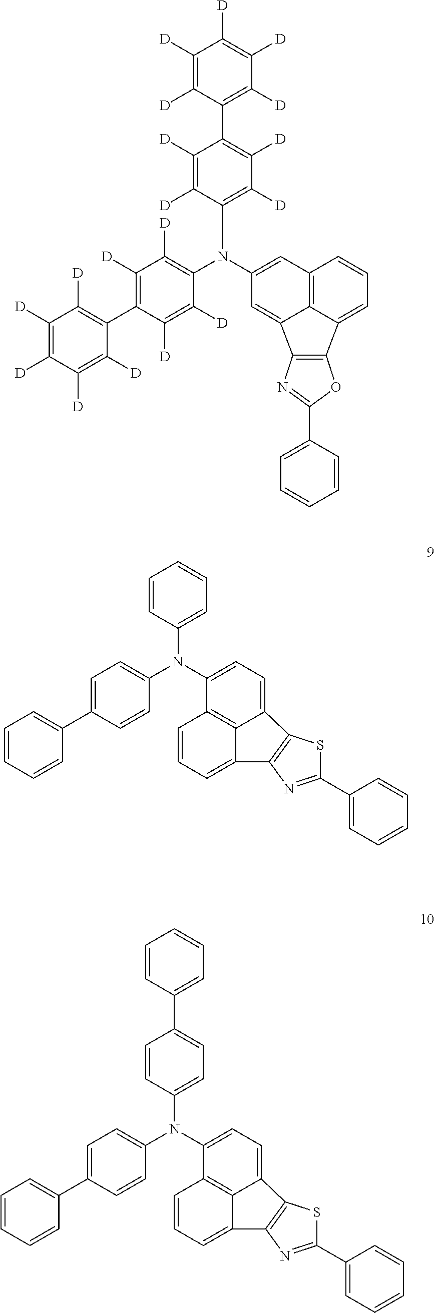 [SMILES: [2H]c1c([2H])c([2H])c(-c2c([2H])c([2H])c(N(c3cc4c5c(cccc5c3)-c3oc(-c5ccccc5)nc3-4)c3c([2H])c([2H])c(-c4c([2H])c([2H])c([2H])c([2H])c4[2H])c([2H])c3[2H])c([2H])c2[2H])c([2H])c1[2H] ; c1ccc(-c2ccc(N(c3ccccc3)c3ccc4c5c(cccc35)-c3nc(-c5ccccc5)sc3-4)cc2)cc1 ; c1ccc(-c2ccc(N(c3ccc(-c4ccccc4)cc3)c3ccc4c5c(cccc35)-c3nc(-c5ccccc5)sc3-4)cc2)cc1]

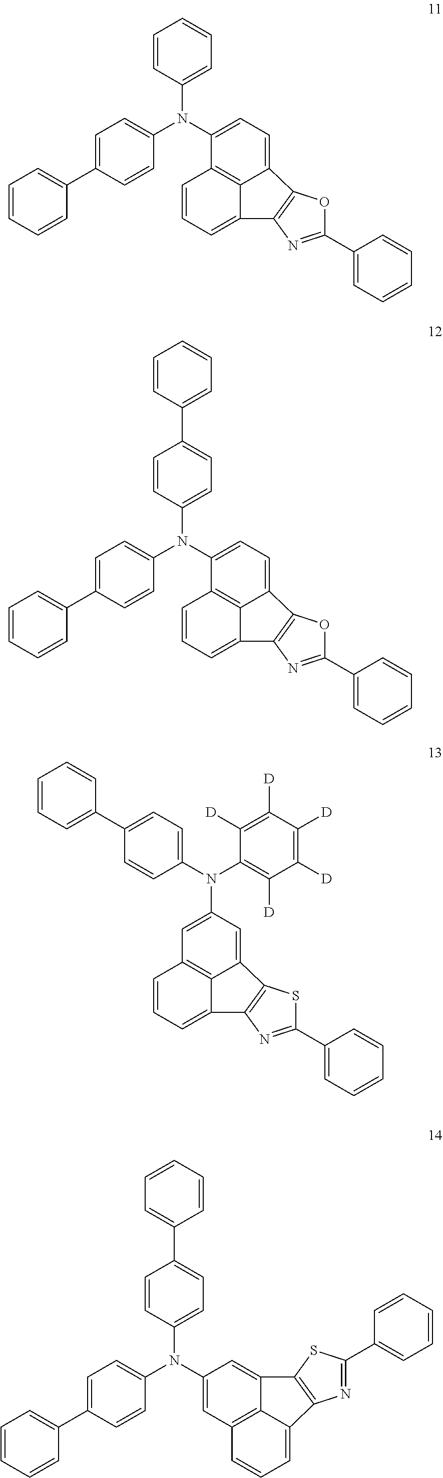 [SMILES: c1ccc(-c2ccc(N(c3ccccc3)c3ccc4c5c(cccc35)-c3nc(-c5ccccc5)oc3-4)cc2)cc1 ; c1ccc(-c2ccc(N(c3ccc(-c4ccccc4)cc3)c3ccc4c5c(cccc35)-c3nc(-c5ccccc5)oc3-4)cc2)cc1 ; [2H]c1c([2H])c([2H])c(N(c2ccc(-c3ccccc3)cc2)c2cc3c4c(cccc4c2)-c2nc(-c4ccccc4)sc2-3)c([2H])c1[2H] ; c1ccc(-c2ccc(N(c3ccc(-c4ccccc4)cc3)c3cc4c5c(cccc5c3)-c3nc(-c5ccccc5)sc3-4)cc2)cc1]

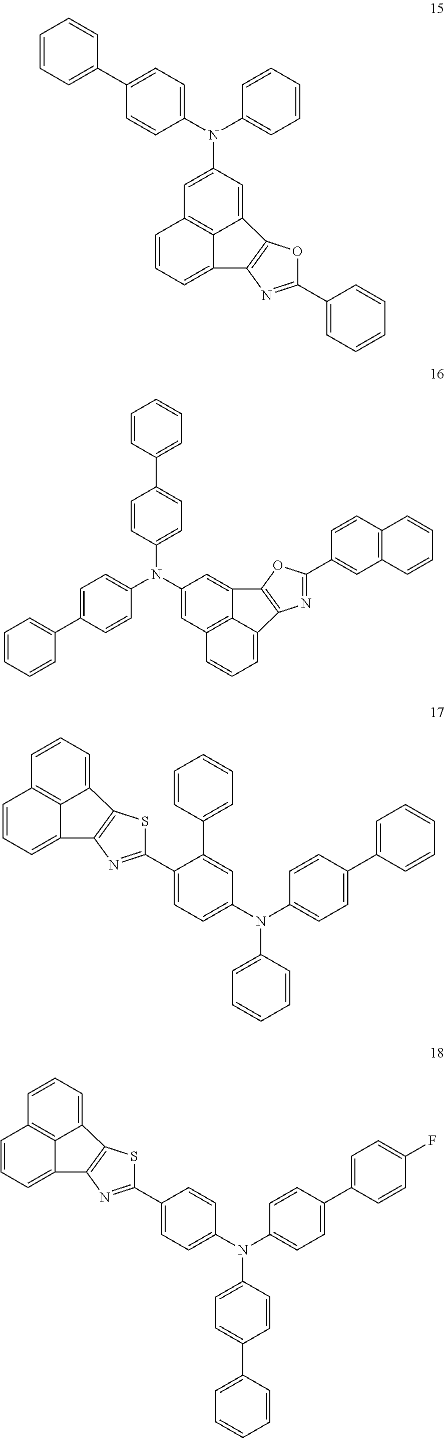 [SMILES: c1ccc(-c2ccc(N(c3ccccc3)c3cc4c5c(cccc5c3)-c3nc(-c5ccccc5)oc3-4)cc2)cc1 ; c1ccc(-c2ccc(N(c3ccc(-c4ccccc4)cc3)c3cc4c5c(cccc5c3)-c3nc(-c5ccc6ccccc6c5)oc3-4)cc2)cc1 ; c1ccc(-c2ccc(N(c3ccccc3)c3ccc(-c4nc5c(s4)-c4cccc6cccc-5c46)c(-c4ccccc4)c3)cc2)cc1 ; Fc1ccc(-c2ccc(N(c3ccc(-c4ccccc4)cc3)c3ccc(-c4nc5c(s4)-c4cccc6cccc-5c46)cc3)cc2)cc1]

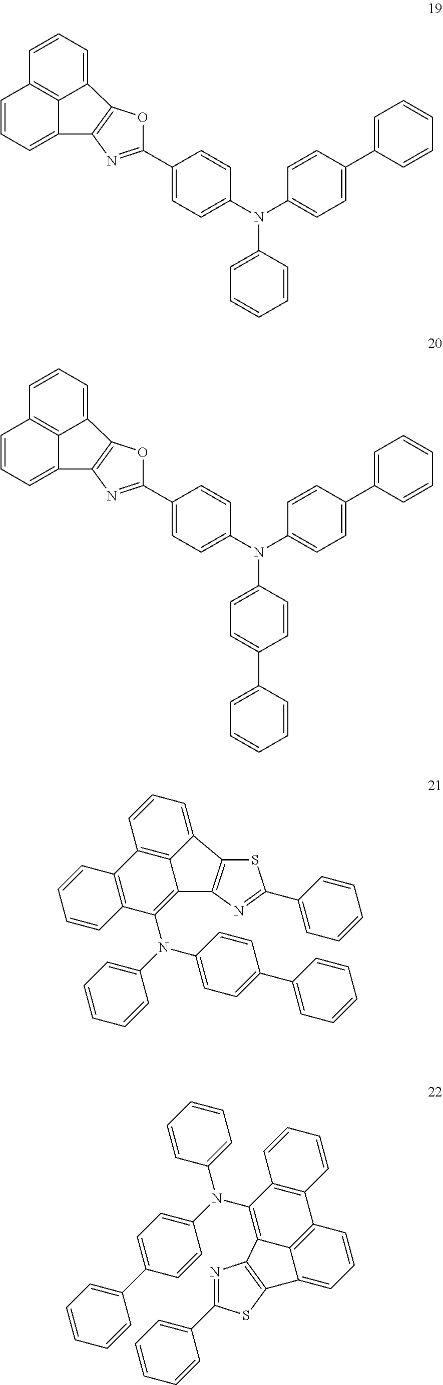 [SMILES: c1ccc(-c2ccc(N(c3ccccc3)c3ccc(-c4nc5c(o4)-c4cccc6cccc-5c46)cc3)cc2)cc1 ; c1ccc(-c2ccc(N(c3ccc(-c4ccccc4)cc3)c3ccc(-c4nc5c(o4)-c4cccc6cccc-5c46)cc3)cc2)cc1 ; c1ccc(-c2ccc(N(c3ccccc3)c3c4c5c(cccc5c5ccccc35)-c3sc(-c5ccccc5)nc3-4)cc2)cc1 ; c1ccc(-c2ccc(N(c3ccccc3)c3c4c5c(cccc5c5ccccc35)-c3sc(-c5ccccc5)nc3-4)cc2)cc1]

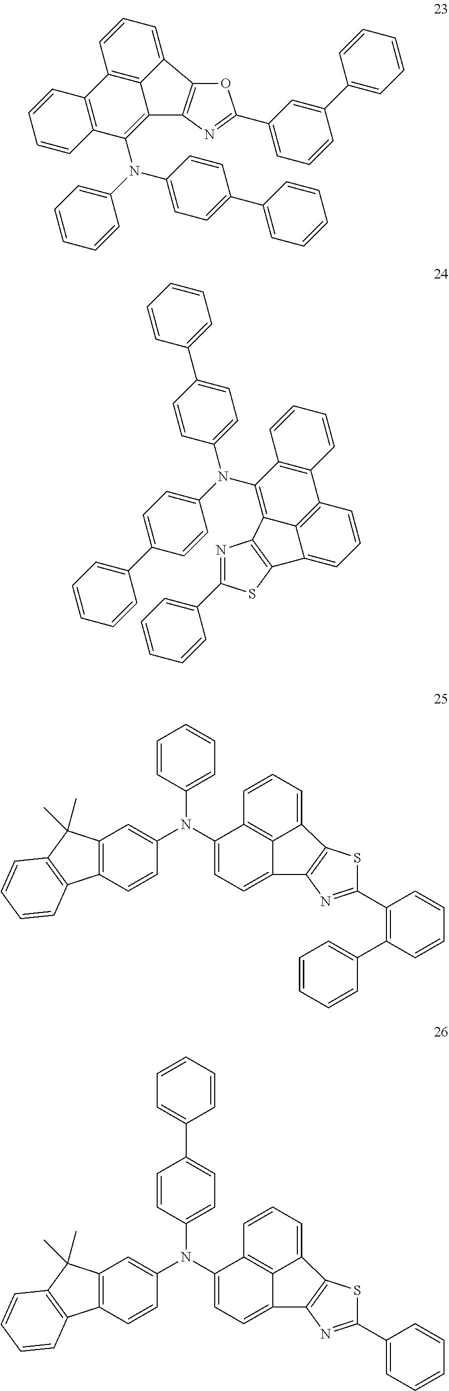 [SMILES: c1ccc(-c2ccc(N(c3ccccc3)c3c4c5c(cccc5c5ccccc35)-c3oc(-c5cccc(-c6ccccc6)c5)nc3-4)cc2)cc1 ; c1ccc(-c2ccc(N(c3ccc(-c4ccccc4)cc3)c3c4c5c(cccc5c5ccccc35)-c3sc(-c5ccccc5)nc3-4)cc2)cc1 ; CC1(C)c2ccccc2-c2ccc(N(c3ccccc3)c3ccc4c5c(cccc35)-c3sc(-c5ccccc5-c5ccccc5)nc3-4)cc21 ; CC1(C)c2ccccc2-c2ccc(N(c3ccc(-c4ccccc4)cc3)c3ccc4c5c(cccc35)-c3sc(-c5ccccc5)nc3-4)cc21]

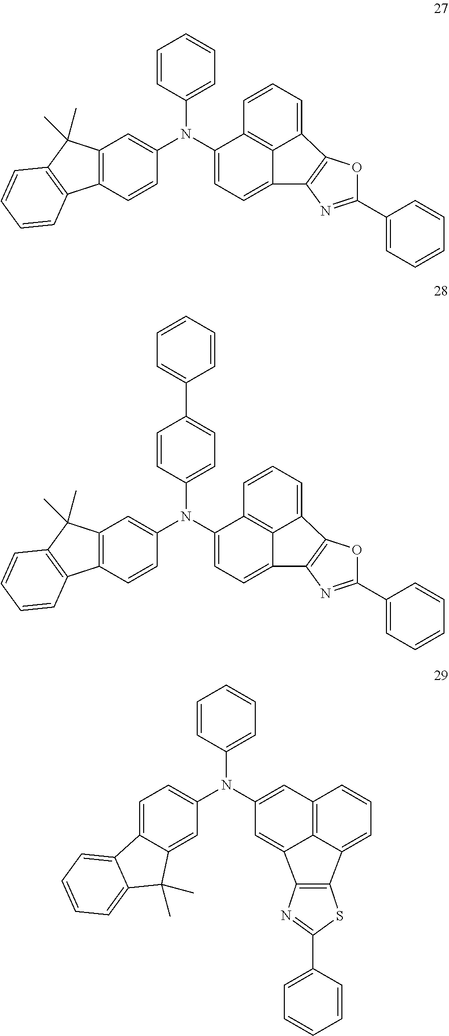 [SMILES: CC1(C)c2ccccc2-c2ccc(N(c3ccccc3)c3ccc4c5c(cccc35)-c3oc(-c5ccccc5)nc3-4)cc21 ; CC1(C)c2ccccc2-c2ccc(N(c3ccc(-c4ccccc4)cc3)c3ccc4c5c(cccc35)-c3oc(-c5ccccc5)nc3-4)cc21 ; CC1(C)c2ccccc2-c2ccc(N(c3ccccc3)c3cc4c5c(cccc5c3)-c3sc(-c5ccccc5)nc3-4)cc21]

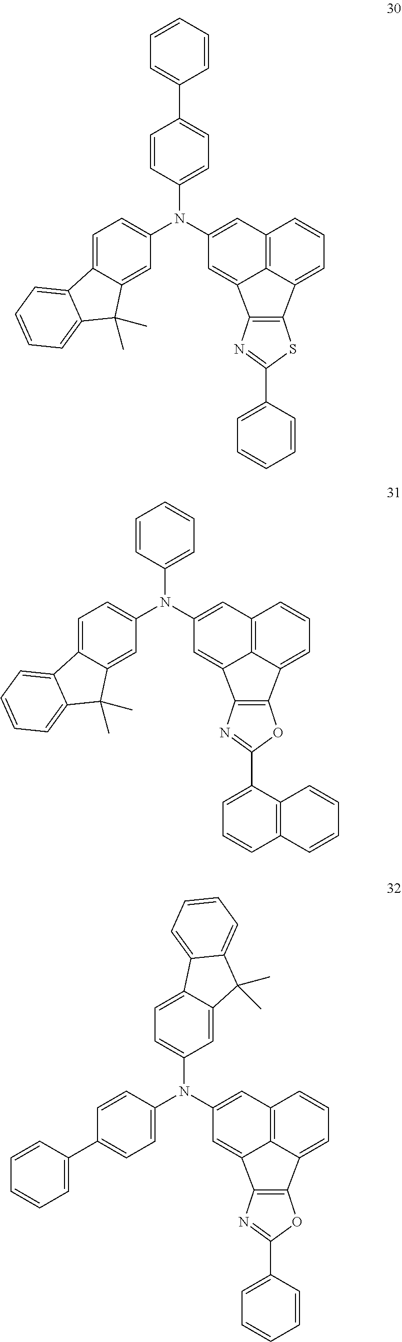 [SMILES: CC1(C)c2ccccc2-c2ccc(N(c3ccc(-c4ccccc4)cc3)c3cc4c5c(cccc5c3)-c3sc(-c5ccccc5)nc3-4)cc21 ; CC1(C)c2ccccc2-c2ccc(N(c3ccccc3)c3cc4c5c(cccc5c3)-c3oc(-c5cccc6ccccc56)nc3-4)cc21 ; CC1(C)c2ccccc2-c2ccc(N(c3ccc(-c4ccccc4)cc3)c3cc4c5c(cccc5c3)-c3oc(-c5ccccc5)nc3-4)cc21]

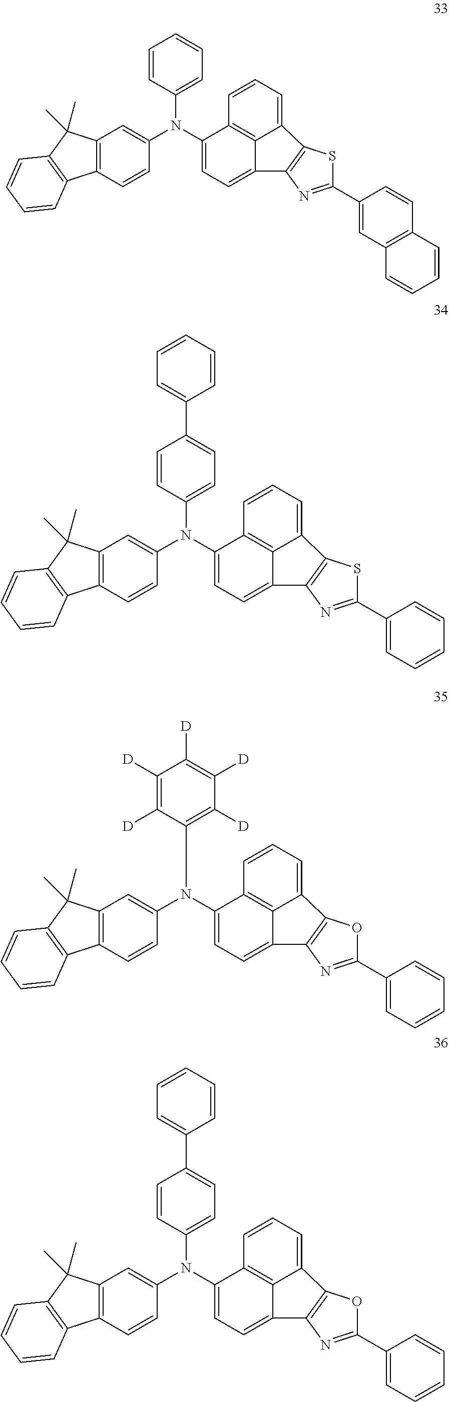 [SMILES: CC1(C)c2ccccc2-c2ccc(N(c3ccccc3)c3ccc4c5c(cccc35)-c3sc(-c5ccc6ccccc6c5)nc3-4)cc21 ; CC1(C)c2ccccc2-c2ccc(N(c3ccc(-c4ccccc4)cc3)c3ccc4c5c(cccc35)-c3sc(-c5ccccc5)nc3-4)cc21 ; [2H]c1c([2H])c([2H])c(N(c2ccc3c(c2)C(C)(C)c2ccccc2-3)c2ccc3c4c(cccc24)-c2oc(-c4ccccc4)nc2-3)c([2H])c1[2H] ; CC1(C)c2ccccc2-c2ccc(N(c3ccc(-c4ccccc4)cc3)c3ccc4c5c(cccc35)-c3oc(-c5ccccc5)nc3-4)cc21]

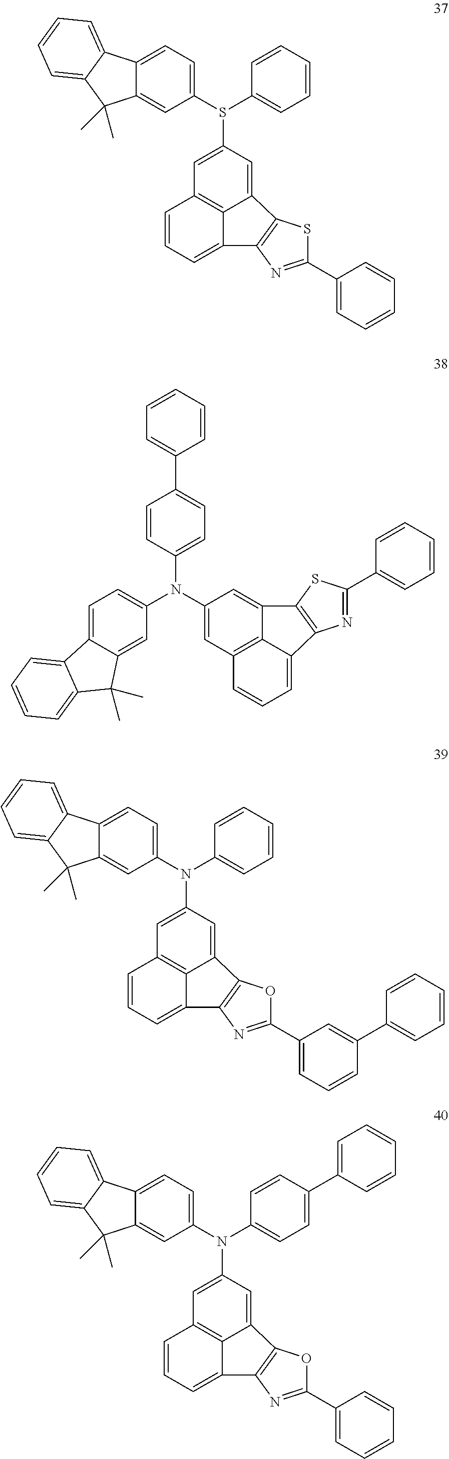

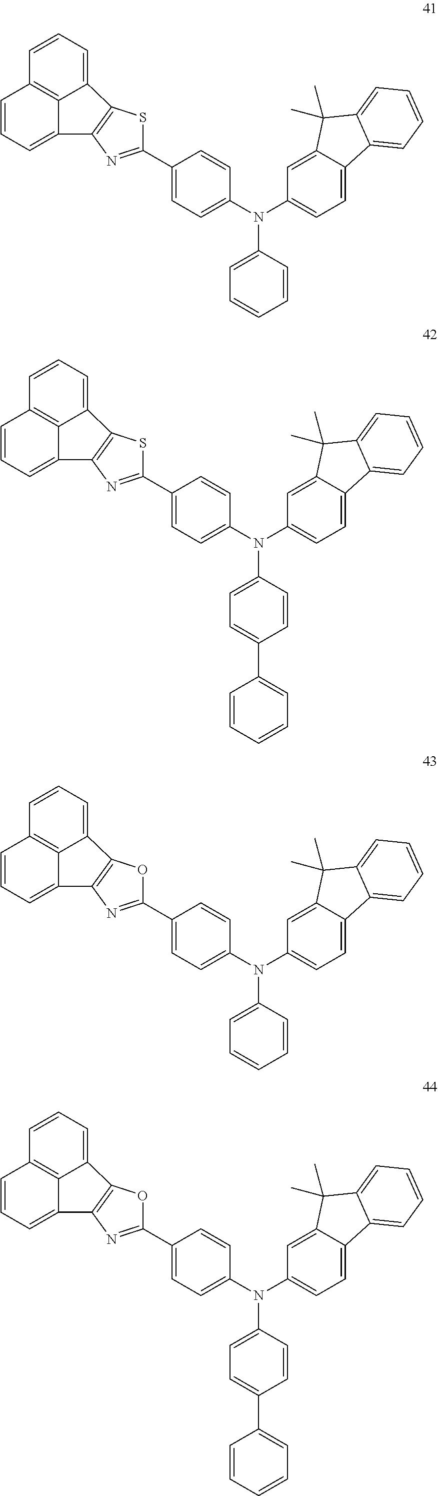 [SMILES: CC1(C)c2ccccc2-c2ccc(N(c3ccccc3)c3ccc(-c4nc5c(s4)-c4cccc6cccc-5c46)cc3)cc21 ; CC1(C)c2ccccc2-c2ccc(N(c3ccc(-c4ccccc4)cc3)c3ccc(-c4nc5c(s4)-c4cccc6cccc-5c46)cc3)cc21 ; CC1(C)c2ccccc2-c2ccc(N(c3ccccc3)c3ccc(-c4nc5c(o4)-c4cccc6cccc-5c46)cc3)cc21 ; CC1(C)c2ccccc2-c2ccc(N(c3ccc(-c4ccccc4)cc3)c3ccc(-c4nc5c(o4)-c4cccc6cccc-5c46)cc3)cc21]

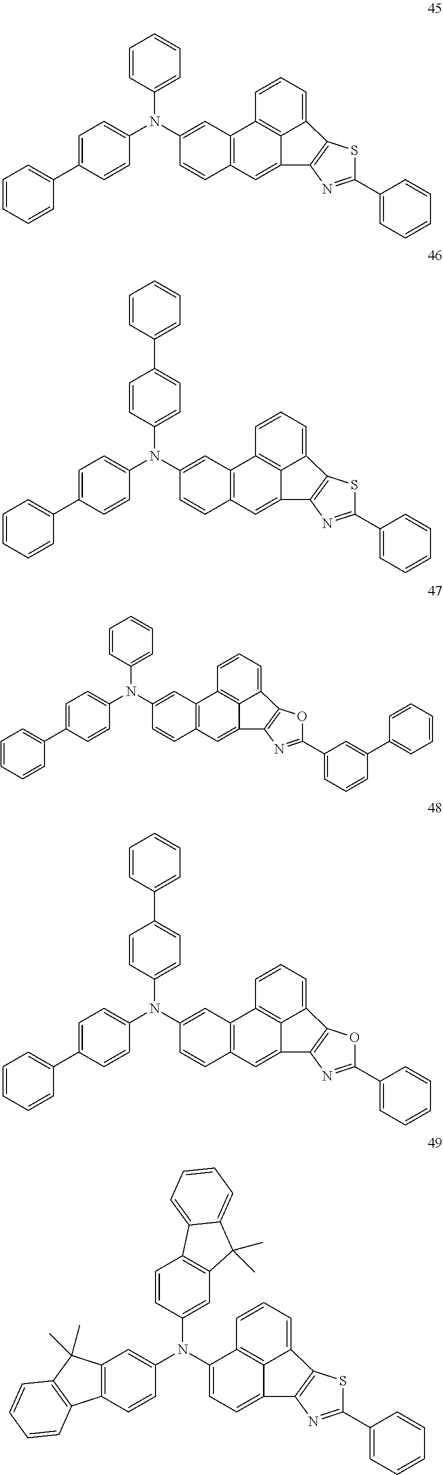 [SMILES: c1ccc(-c2ccc(N(c3ccccc3)c3ccc4cc5c6c(cccc6c4c3)-c3sc(-c4ccccc4)nc3-5)cc2)cc1 ; c1ccc(-c2ccc(N(c3ccc(-c4ccccc4)cc3)c3ccc4cc5c6c(cccc6c4c3)-c3sc(-c4ccccc4)nc3-5)cc2)cc1 ; c1ccc(-c2ccc(N(c3ccccc3)c3ccc4cc5c6c(cccc6c4c3)-c3oc(-c4cccc(-c6ccccc6)c4)nc3-5)cc2)cc1 ; c1ccc(-c2ccc(N(c3ccc(-c4ccccc4)cc3)c3ccc4cc5c6c(cccc6c4c3)-c3oc(-c4ccccc4)nc3-5)cc2)cc1 ; CC1(C)c2ccccc2-c2ccc(N(c3ccc4c(c3)C(C)(C)c3ccccc3-4)c3ccc4c5c(cccc35)-c3sc(-c5ccccc5)nc3-4)cc21]

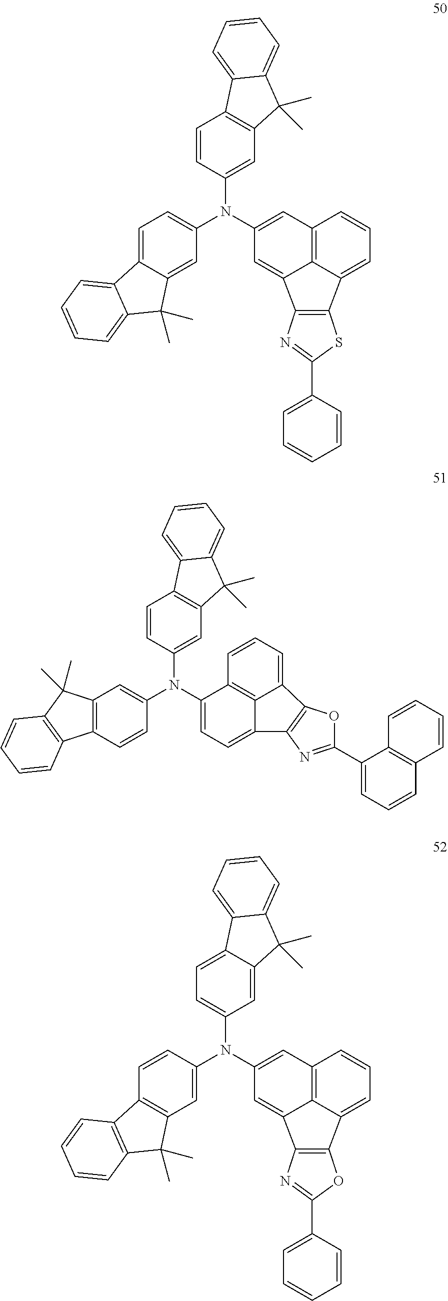 [SMILES: CC1(C)c2ccccc2-c2ccc(N(c3ccc4c(c3)C(C)(C)c3ccccc3-4)c3cc4c5c(cccc5c3)-c3sc(-c5ccccc5)nc3-4)cc21 ; CC1(C)c2ccccc2-c2ccc(N(c3ccc4c(c3)C(C)(C)c3ccccc3-4)c3ccc4c5c(cccc35)-c3oc(-c5cccc6ccccc56)nc3-4)cc21 ; CC1(C)c2ccccc2-c2ccc(N(c3ccc4c(c3)C(C)(C)c3ccccc3-4)c3cc4c5c(cccc5c3)-c3oc(-c5ccccc5)nc3-4)cc21]

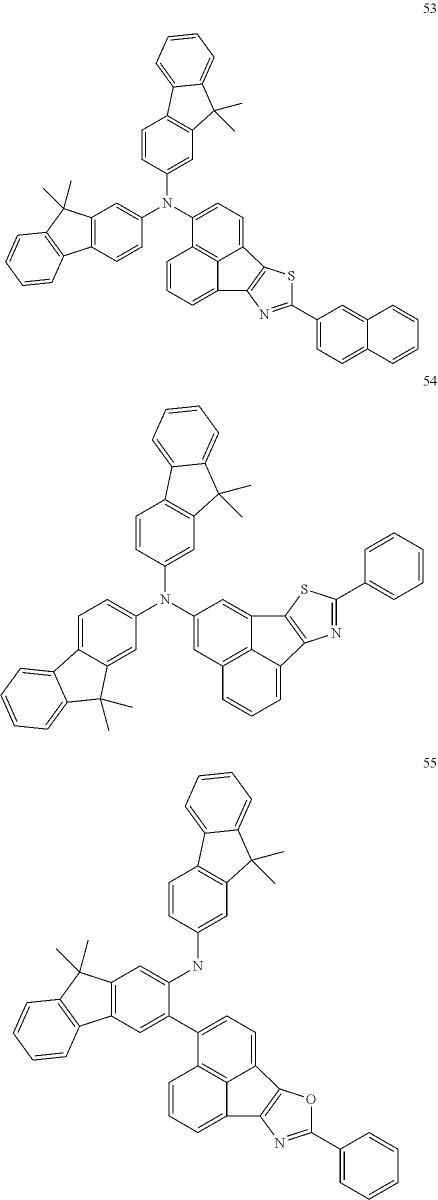 [SMILES: CC1(C)c2ccccc2-c2ccc(N(c3ccc4c(c3)C(C)(C)c3ccccc3-4)c3ccc4c5c(cccc35)-c3nc(-c5ccc6ccccc6c5)sc3-4)cc21 ; CC1(C)c2ccccc2-c2ccc(N(c3ccc4c(c3)C(C)(C)c3ccccc3-4)c3cc4c5c(cccc5c3)-c3nc(-c5ccccc5)sc3-4)cc21 ; CC1(C)c2ccccc2-c2ccc(Nc3cc4c(cc3-c3ccc5c6c(cccc36)-c3nc(-c6ccccc6)oc3-5)-c3ccccc3C4(C)C)cc21]

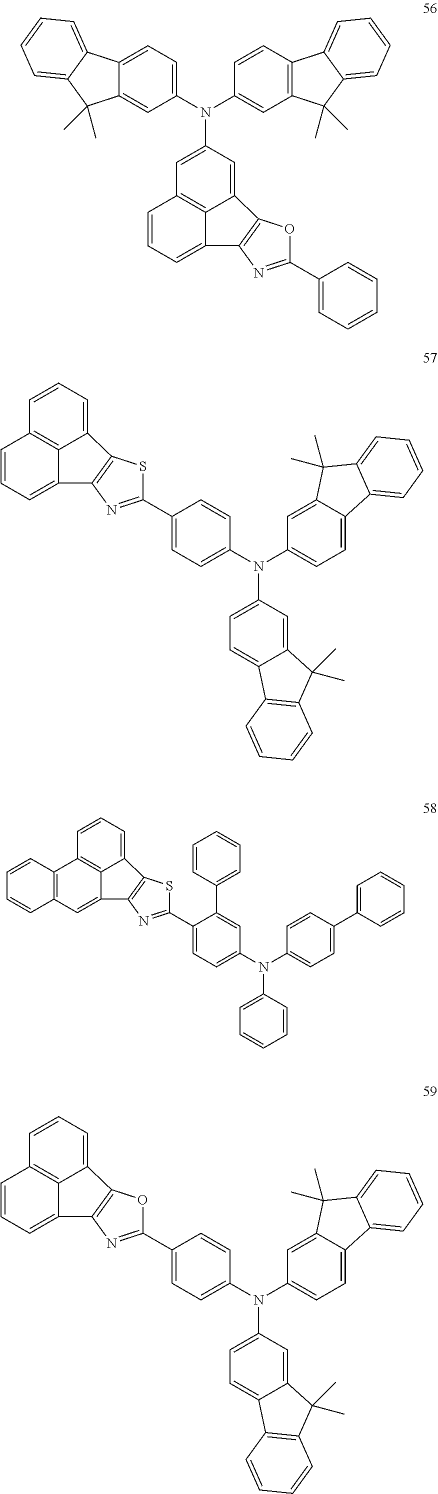 [SMILES: CC1(C)c2ccccc2-c2ccc(N(c3ccc4c(c3)C(C)(C)c3ccccc3-4)c3cc4c5c(cccc5c3)-c3nc(-c5ccccc5)oc3-4)cc21 ; CC1(C)c2ccccc2-c2ccc(N(c3ccc(-c4nc5c(s4)-c4cccc6cccc-5c46)cc3)c3ccc4c(c3)C(C)(C)c3ccccc3-4)cc21 ; c1ccc(-c2ccc(N(c3ccccc3)c3ccc(-c4nc5c(s4)-c4cccc6c4c-5cc4ccccc46)c(-c4ccccc4)c3)cc2)cc1 ; CC1(C)c2ccccc2-c2ccc(N(c3ccc(-c4nc5c(o4)-c4cccc6cccc-5c46)cc3)c3ccc4c(c3)C(C)(C)c3ccccc3-4)cc21]

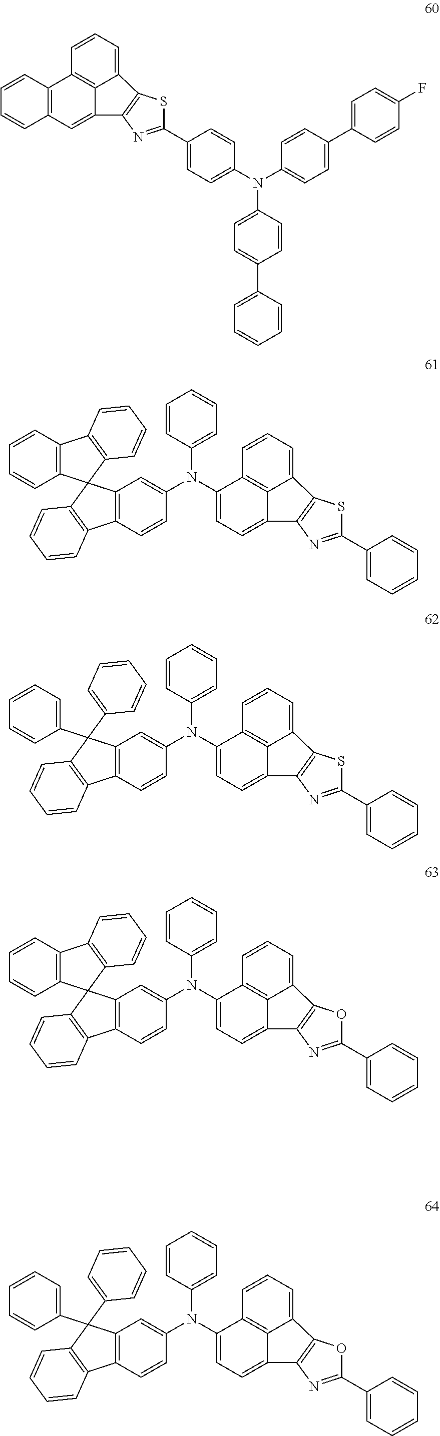 [SMILES: Fc1ccc(-c2ccc(N(c3ccc(-c4ccccc4)cc3)c3ccc(-c4nc5c(s4)-c4cccc6c4c-5cc4ccccc46)cc3)cc2)cc1 ; c1ccc(-c2nc3c(s2)-c2cccc4c(N(c5ccccc5)c5ccc6c(c5)C5(c7ccccc7-c7ccccc75)c5ccccc5-6)ccc-3c24)cc1 ; c1ccc(-c2nc3c(s2)-c2cccc4c(N(c5ccccc5)c5ccc6c(c5)C(c5ccccc5)(c5ccccc5)c5ccccc5-6)ccc-3c24)cc1 ; c1ccc(-c2nc3c(o2)-c2cccc4c(N(c5ccccc5)c5ccc6c(c5)C5(c7ccccc7-c7ccccc75)c5ccccc5-6)ccc-3c24)cc1 ; c1ccc(-c2nc3c(o2)-c2cccc4c(N(c5ccccc5)c5ccc6c(c5)C(c5ccccc5)(c5ccccc5)c5ccccc5-6)ccc-3c24)cc1]

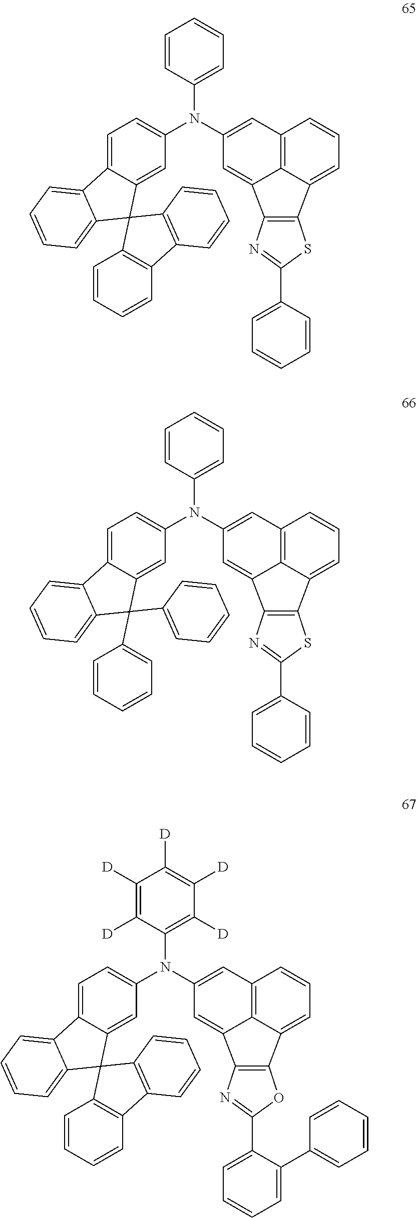 [SMILES: c1ccc(-c2nc3c(s2)-c2cccc4cc(N(c5ccccc5)c5ccc6c(c5)C5(c7ccccc7-c7ccccc75)c5ccccc5-6)cc-3c24)cc1 ; c1ccc(-c2nc3c(s2)-c2cccc4cc(N(c5ccccc5)c5ccc6c(c5)C(c5ccccc5)(c5ccccc5)c5ccccc5-6)cc-3c24)cc1 ; [2H]c1c([2H])c([2H])c(N(c2ccc3c(c2)C2(c4ccccc4-c4ccccc42)c2ccccc2-3)c2cc3c4c(cccc4c2)-c2oc(-c4ccccc4-c4ccccc4)nc2-3)c([2H])c1[2H]]

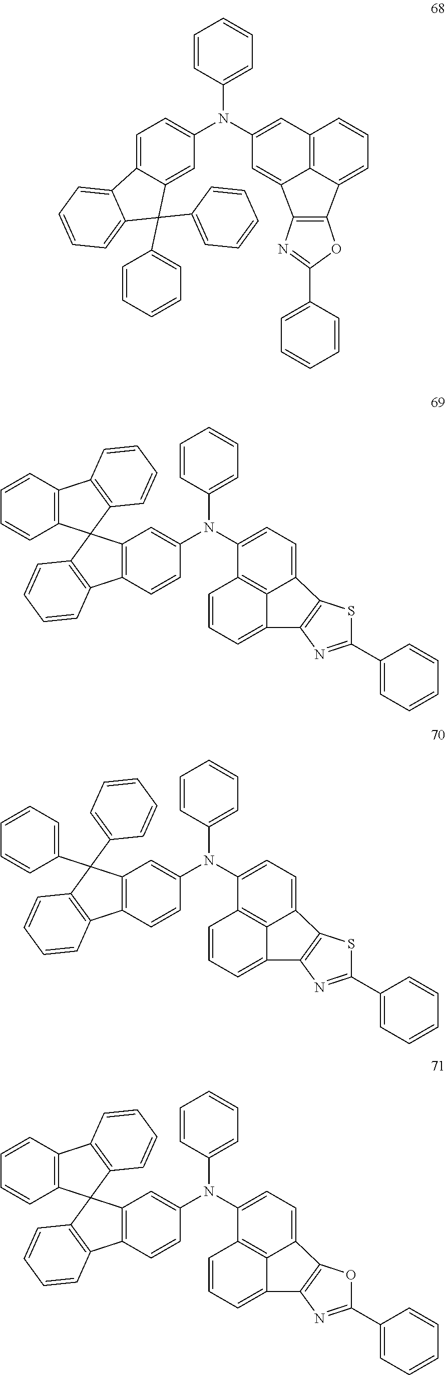 [SMILES: c1ccc(-c2nc3c(o2)-c2cccc4cc(N(c5ccccc5)c5ccc6c(c5)C(c5ccccc5)(c5ccccc5)c5ccccc5-6)cc-3c24)cc1 ; c1ccc(-c2nc3c(s2)-c2ccc(N(c4ccccc4)c4ccc5c(c4)C4(c6ccccc6-c6ccccc64)c4ccccc4-5)c4cccc-3c24)cc1 ; c1ccc(-c2nc3c(s2)-c2ccc(N(c4ccccc4)c4ccc5c(c4)C(c4ccccc4)(c4ccccc4)c4ccccc4-5)c4cccc-3c24)cc1 ; c1ccc(-c2nc3c(o2)-c2ccc(N(c4ccccc4)c4ccc5c(c4)C4(c6ccccc6-c6ccccc64)c4ccccc4-5)c4cccc-3c24)cc1]

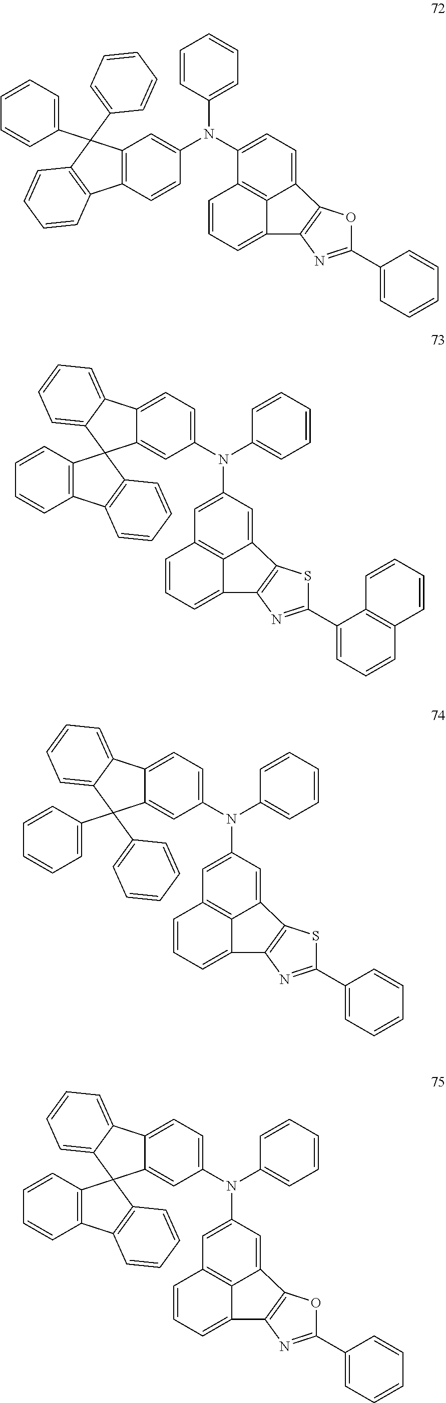 [SMILES: c1ccc(-c2nc3c(o2)-c2ccc(N(c4ccccc4)c4ccc5c(c4)C(c4ccccc4)(c4ccccc4)c4ccccc4-5)c4cccc-3c24)cc1 ; c1ccc(N(c2ccc3c(c2)C2(c4ccccc4-c4ccccc42)c2ccccc2-3)c2cc3c4c(cccc4c2)-c2nc(-c4cccc5ccccc45)sc2-3)cc1 ; c1ccc(-c2nc3c(s2)-c2cc(N(c4ccccc4)c4ccc5c(c4)C(c4ccccc4)(c4ccccc4)c4ccccc4-5)cc4cccc-3c24)cc1 ; c1ccc(-c2nc3c(o2)-c2cc(N(c4ccccc4)c4ccc5c(c4)C4(c6ccccc6-c6ccccc64)c4ccccc4-5)cc4cccc-3c24)cc1]

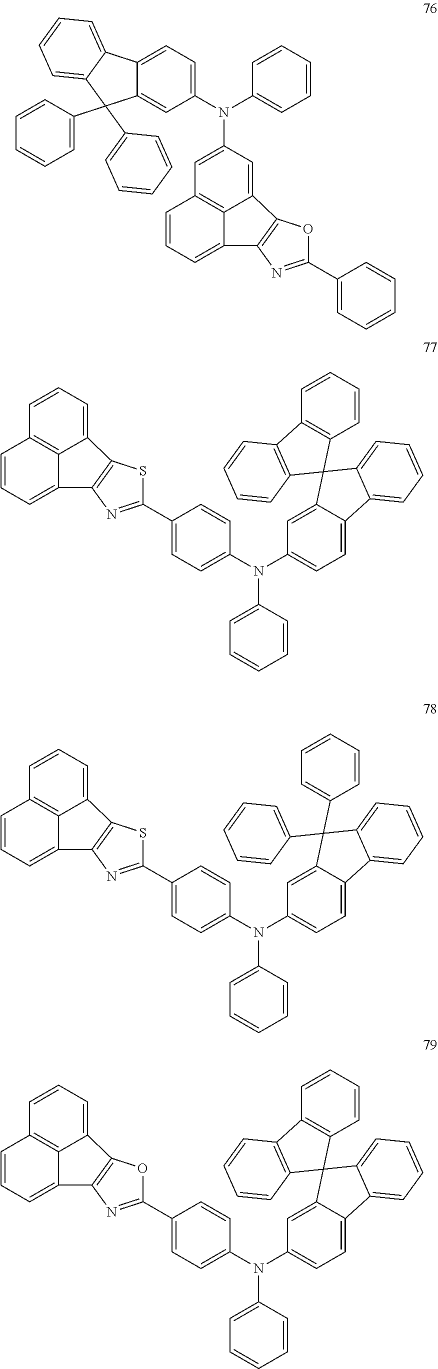 [SMILES: c1ccc(-c2nc3c(o2)-c2cc(N(c4ccccc4)c4ccc5c(c4)C(c4ccccc4)(c4ccccc4)c4ccccc4-5)cc4cccc-3c24)cc1 ; c1ccc(N(c2ccc(-c3nc4c(s3)-c3cccc5cccc-4c35)cc2)c2ccc3c(c2)C2(c4ccccc4-c4ccccc42)c2ccccc2-3)cc1 ; c1ccc(N(c2ccc(-c3nc4c(s3)-c3cccc5cccc-4c35)cc2)c2ccc3c(c2)C(c2ccccc2)(c2ccccc2)c2ccccc2-3)cc1 ; c1ccc(N(c2ccc(-c3nc4c(o3)-c3cccc5cccc-4c35)cc2)c2ccc3c(c2)C2(c4ccccc4-c4ccccc42)c2ccccc2-3)cc1]

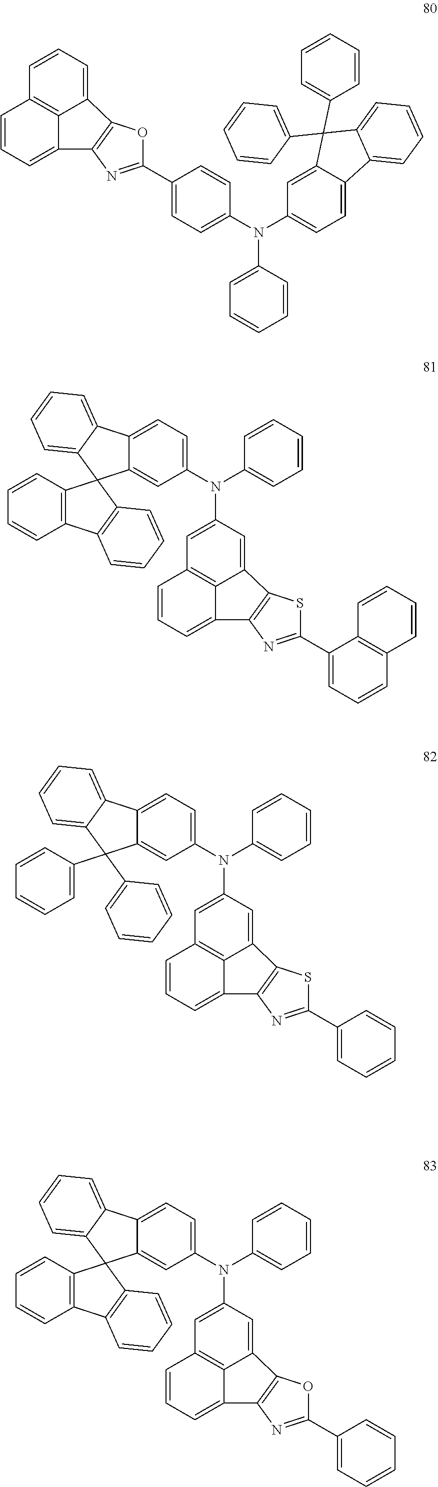 [SMILES: c1ccc(N(c2ccc(-c3nc4c(o3)-c3cccc5cccc-4c35)cc2)c2ccc3c(c2)C(c2ccccc2)(c2ccccc2)c2ccccc2-3)cc1 ; c1ccc(N(c2ccc3c(c2)C2(c4ccccc4-c4ccccc42)c2ccccc2-3)c2cc3c4c(cccc4c2)-c2nc(-c4cccc5ccccc45)sc2-3)cc1 ; c1ccc(-c2nc3c(s2)-c2cc(N(c4ccccc4)c4ccc5c(c4)C(c4ccccc4)(c4ccccc4)c4ccccc4-5)cc4cccc-3c24)cc1 ; c1ccc(-c2nc3c(o2)-c2cc(N(c4ccccc4)c4ccc5c(c4)C4(c6ccccc6-c6ccccc64)c4ccccc4-5)cc4cccc-3c24)cc1]

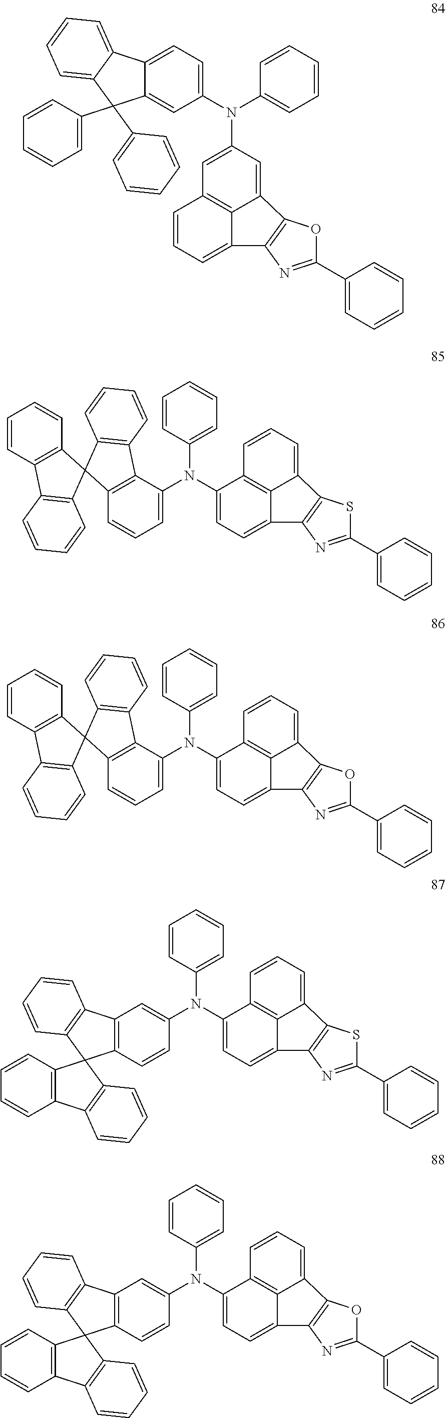 [SMILES: c1ccc(-c2nc3c(o2)-c2cc(N(c4ccccc4)c4ccc5c(c4)C(c4ccccc4)(c4ccccc4)c4ccccc4-5)cc4cccc-3c24)cc1 ; c1ccc(-c2nc3c(s2)-c2cccc4c(N(c5ccccc5)c5cccc6c5-c5ccccc5C65c6ccccc6-c6ccccc65)ccc-3c24)cc1 ; c1ccc(-c2nc3c(o2)-c2cccc4c(N(c5ccccc5)c5cccc6c5-c5ccccc5C65c6ccccc6-c6ccccc65)ccc-3c24)cc1 ; c1ccc(-c2nc3c(s2)-c2cccc4c(N(c5ccccc5)c5ccc6c(c5)-c5ccccc5C65c6ccccc6-c6ccccc65)ccc-3c24)cc1 ; c1ccc(-c2nc3c(o2)-c2cccc4c(N(c5ccccc5)c5ccc6c(c5)-c5ccccc5C65c6ccccc6-c6ccccc65)ccc-3c24)cc1]

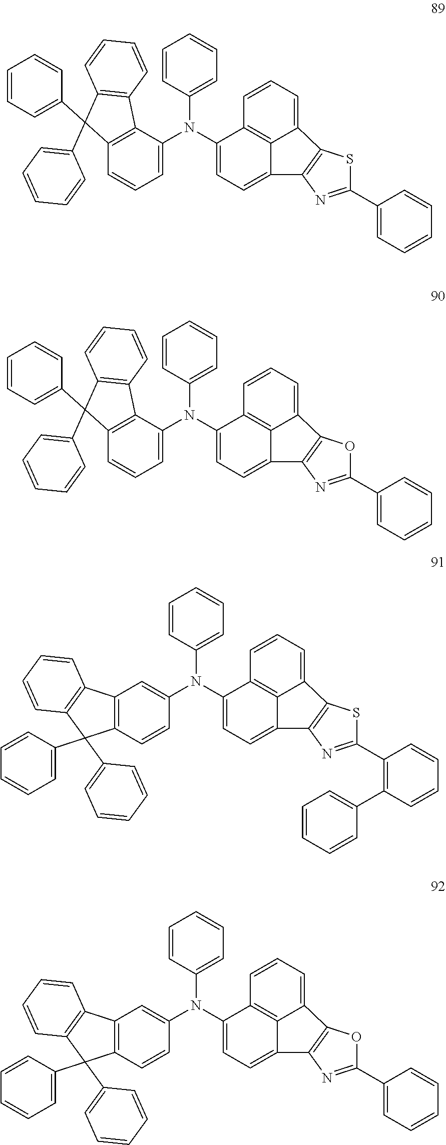 [SMILES: c1ccc(-c2nc3c(s2)-c2cccc4c(N(c5ccccc5)c5cccc6c5-c5ccccc5C6(c5ccccc5)c5ccccc5)ccc-3c24)cc1 ; c1ccc(-c2nc3c(o2)-c2cccc4c(N(c5ccccc5)c5cccc6c5-c5ccccc5C6(c5ccccc5)c5ccccc5)ccc-3c24)cc1 ; c1ccc(-c2ccccc2-c2nc3c(s2)-c2cccc4c(N(c5ccccc5)c5ccc6c(c5)-c5ccccc5C6(c5ccccc5)c5ccccc5)ccc-3c24)cc1 ; c1ccc(-c2nc3c(o2)-c2cccc4c(N(c5ccccc5)c5ccc6c(c5)-c5ccccc5C6(c5ccccc5)c5ccccc5)ccc-3c24)cc1]

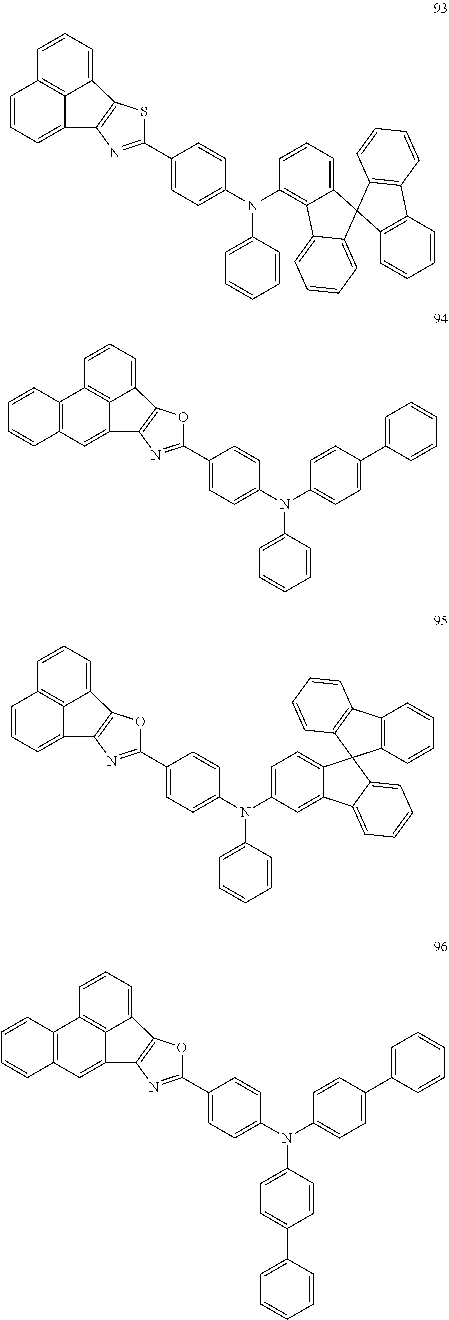 [SMILES: c1ccc(N(c2ccc(-c3nc4c(s3)-c3cccc5cccc-4c35)cc2)c2cccc3c2-c2ccccc2C32c3ccccc3-c3ccccc32)cc1 ; c1ccc(-c2ccc(N(c3ccccc3)c3ccc(-c4nc5c(o4)-c4cccc6c4c-5cc4ccccc46)cc3)cc2)cc1 ; c1ccc(N(c2ccc(-c3nc4c(o3)-c3cccc5cccc-4c35)cc2)c2ccc3c(c2)-c2ccccc2C32c3ccccc3-c3ccccc32)cc1 ; c1ccc(-c2ccc(N(c3ccc(-c4ccccc4)cc3)c3ccc(-c4nc5c(o4)-c4cccc6c4c-5cc4ccccc46)cc3)cc2)cc1]

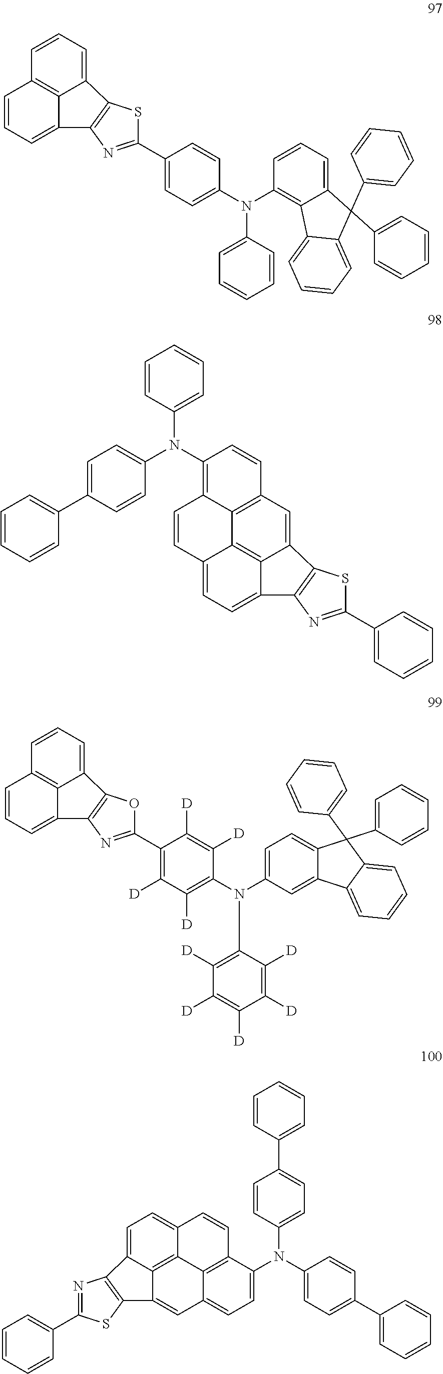 [SMILES: c1ccc(N(c2ccc(-c3nc4c(s3)-c3cccc5cccc-4c35)cc2)c2cccc3c2-c2ccccc2C3(c2ccccc2)c2ccccc2)cc1 ; c1ccc(-c2ccc(N(c3ccccc3)c3ccc4cc5c6c(ccc7ccc3c4c76)-c3nc(-c4ccccc4)sc3-5)cc2)cc1 ; [2H]c1c([2H])c([2H])c(N(c2ccc3c(c2)-c2ccccc2C3(c2ccccc2)c2ccccc2)c2c([2H])c([2H])c(-c3nc4c(o3)-c3cccc5cccc-4c35)c([2H])c2[2H])c([2H])c1[2H] ; c1ccc(-c2ccc(N(c3ccc(-c4ccccc4)cc3)c3ccc4cc5c6c(ccc7ccc3c4c76)-c3nc(-c4ccccc4)sc3-5)cc2)cc1]

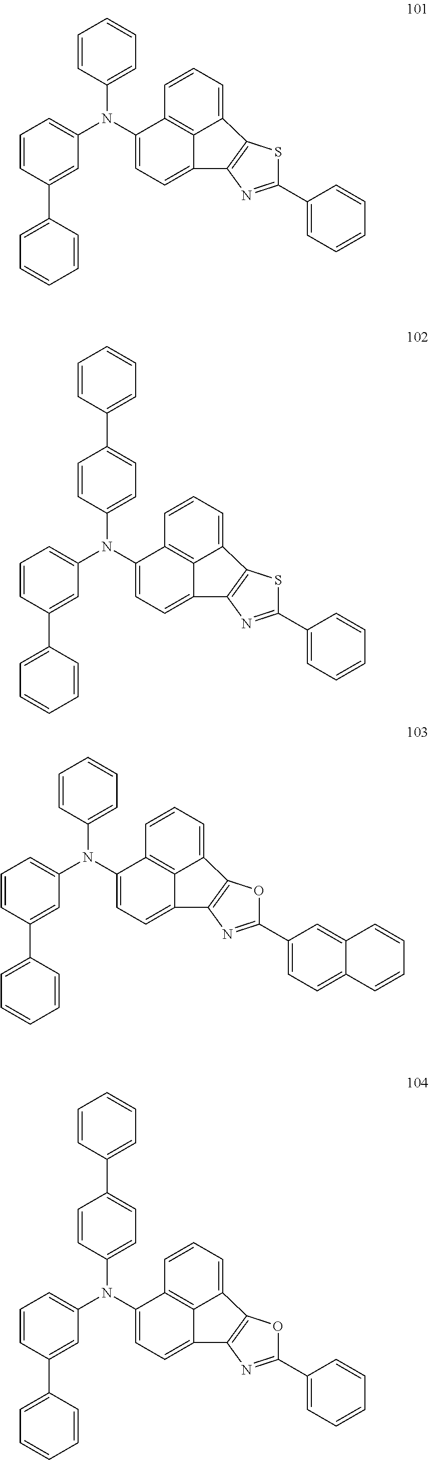 [SMILES: c1ccc(-c2cccc(N(c3ccccc3)c3ccc4c5c(cccc35)-c3sc(-c5ccccc5)nc3-4)c2)cc1 ; c1ccc(-c2ccc(N(c3cccc(-c4ccccc4)c3)c3ccc4c5c(cccc35)-c3sc(-c5ccccc5)nc3-4)cc2)cc1 ; c1ccc(-c2cccc(N(c3ccccc3)c3ccc4c5c(cccc35)-c3oc(-c5ccc6ccccc6c5)nc3-4)c2)cc1 ; c1ccc(-c2ccc(N(c3cccc(-c4ccccc4)c3)c3ccc4c5c(cccc35)-c3oc(-c5ccccc5)nc3-4)cc2)cc1]

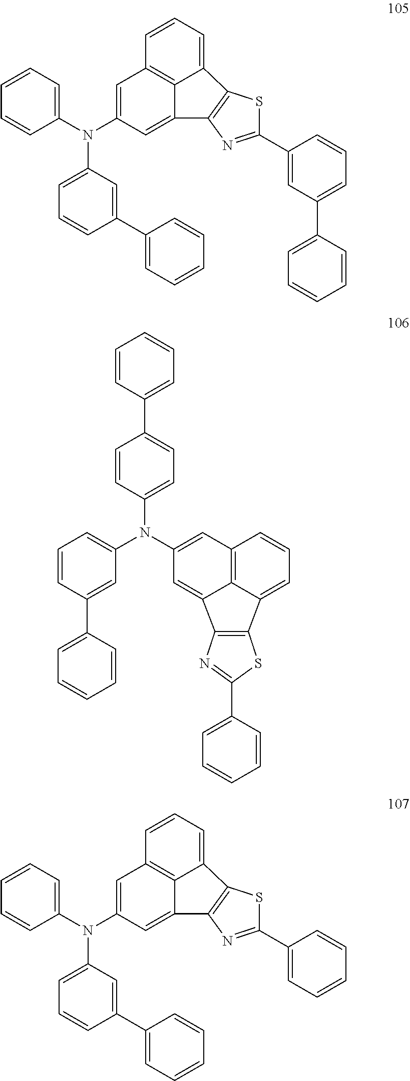 [SMILES: c1ccc(-c2cccc(-c3nc4c(s3)-c3cccc5cc(N(c6ccccc6)c6cccc(-c7ccccc7)c6)cc-4c35)c2)cc1 ; c1ccc(-c2ccc(N(c3cccc(-c4ccccc4)c3)c3cc4c5c(cccc5c3)-c3sc(-c5ccccc5)nc3-4)cc2)cc1 ; c1ccc(-c2cccc(N(c3ccccc3)c3cc4c5c(cccc5c3)-c3sc(-c5ccccc5)nc3-4)c2)cc1]

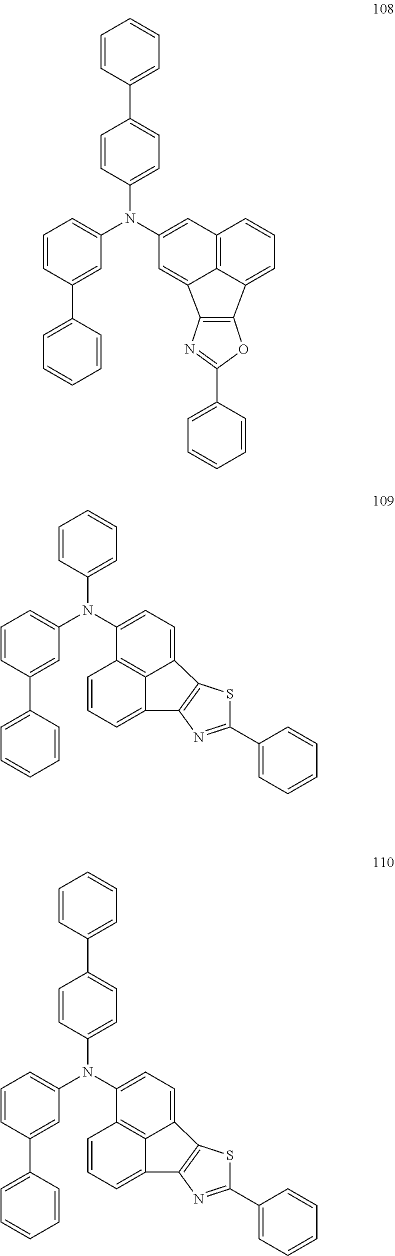 [SMILES: c1ccc(-c2ccc(N(c3cccc(-c4ccccc4)c3)c3cc4c5c(cccc5c3)-c3oc(-c5ccccc5)nc3-4)cc2)cc1 ; c1ccc(-c2cccc(N(c3ccccc3)c3ccc4c5c(cccc35)-c3nc(-c5ccccc5)sc3-4)c2)cc1 ; c1ccc(-c2ccc(N(c3cccc(-c4ccccc4)c3)c3ccc4c5c(cccc35)-c3nc(-c5ccccc5)sc3-4)cc2)cc1]

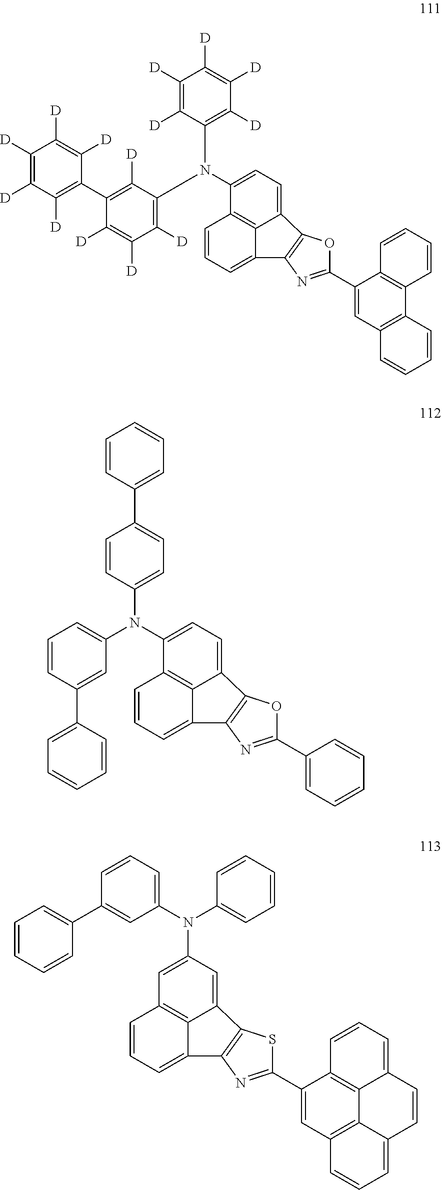 [SMILES: [2H]c1c([2H])c([2H])c(-c2c([2H])c([2H])c([2H])c(N(c3c([2H])c([2H])c([2H])c([2H])c3[2H])c3ccc4c5c(cccc35)-c3nc(-c5cc6ccccc6c6ccccc56)oc3-4)c2[2H])c([2H])c1[2H] ; c1ccc(-c2ccc(N(c3cccc(-c4ccccc4)c3)c3ccc4c5c(cccc35)-c3nc(-c5ccccc5)oc3-4)cc2)cc1 ; c1ccc(-c2cccc(N(c3ccccc3)c3cc4c5c(cccc5c3)-c3nc(-c5cc6cccc7ccc8cccc5c8c76)sc3-4)c2)cc1]

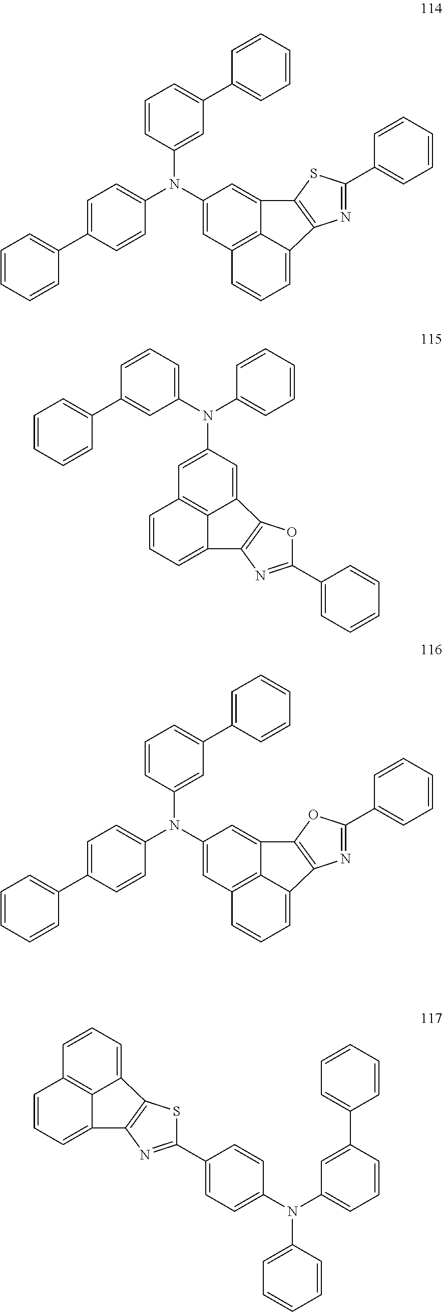 [SMILES: c1ccc(-c2ccc(N(c3cccc(-c4ccccc4)c3)c3cc4c5c(cccc5c3)-c3nc(-c5ccccc5)sc3-4)cc2)cc1 ; c1ccc(-c2cccc(N(c3ccccc3)c3cc4c5c(cccc5c3)-c3nc(-c5ccccc5)oc3-4)c2)cc1 ; c1ccc(-c2ccc(N(c3cccc(-c4ccccc4)c3)c3cc4c5c(cccc5c3)-c3nc(-c5ccccc5)oc3-4)cc2)cc1 ; c1ccc(-c2cccc(N(c3ccccc3)c3ccc(-c4nc5c(s4)-c4cccc6cccc-5c46)cc3)c2)cc1]

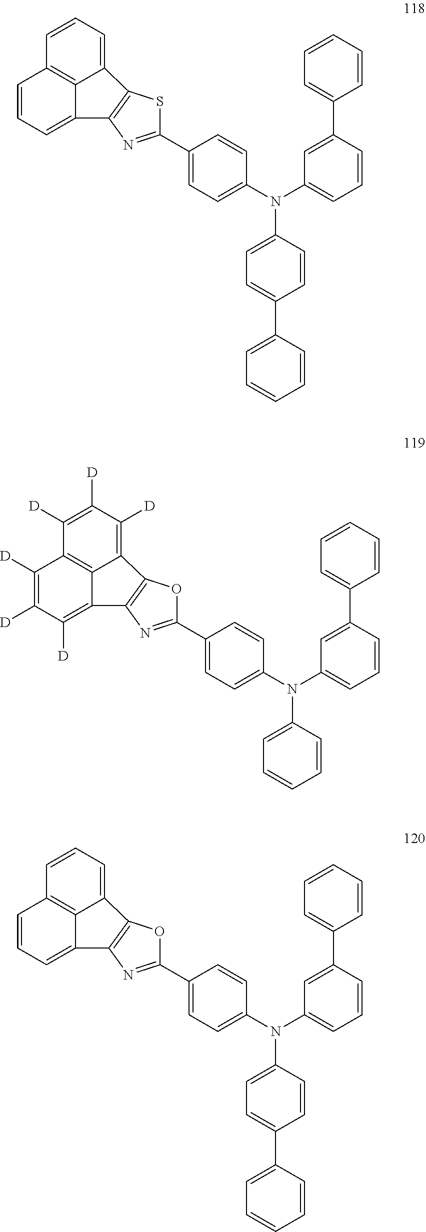 [SMILES: c1ccc(-c2ccc(N(c3ccc(-c4nc5c(s4)-c4cccc6cccc-5c46)cc3)c3cccc(-c4ccccc4)c3)cc2)cc1 ; [2H]c1c([2H])c2c3c(c([2H])c([2H])c([2H])c3c1[2H])-c1oc(-c3ccc(N(c4ccccc4)c4cccc(-c5ccccc5)c4)cc3)nc1-2 ; c1ccc(-c2ccc(N(c3ccc(-c4nc5c(o4)-c4cccc6cccc-5c46)cc3)c3cccc(-c4ccccc4)c3)cc2)cc1]

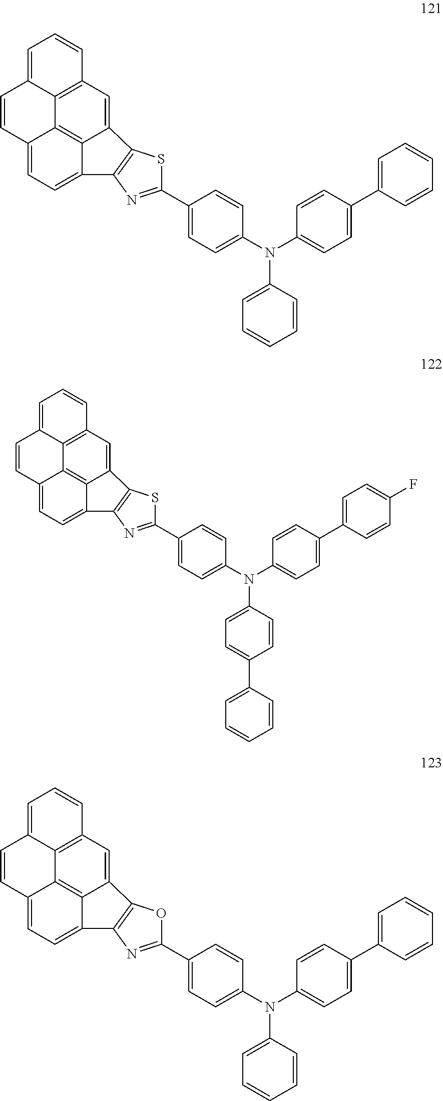 [SMILES: c1ccc(-c2ccc(N(c3ccccc3)c3ccc(-c4nc5c(s4)-c4cc6cccc7ccc8ccc-5c4c8c76)cc3)cc2)cc1 ; Fc1ccc(-c2ccc(N(c3ccc(-c4ccccc4)cc3)c3ccc(-c4nc5c(s4)-c4cc6cccc7ccc8ccc-5c4c8c76)cc3)cc2)cc1 ; c1ccc(-c2ccc(N(c3ccccc3)c3ccc(-c4nc5c(o4)-c4cc6cccc7ccc8ccc-5c4c8c76)cc3)cc2)cc1]

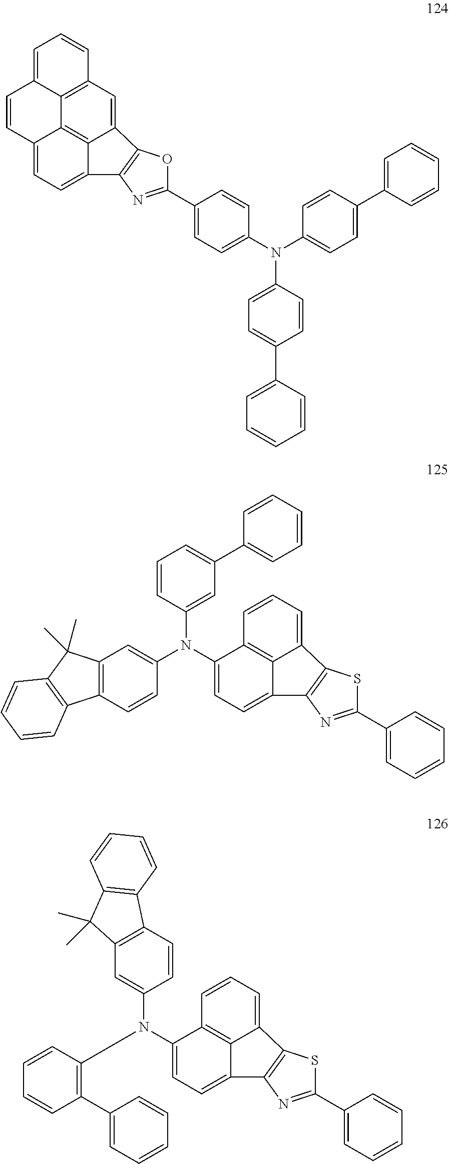 [SMILES: c1ccc(-c2ccc(N(c3ccc(-c4ccccc4)cc3)c3ccc(-c4nc5c(o4)-c4cc6cccc7ccc8ccc-5c4c8c76)cc3)cc2)cc1 ; CC1(C)c2ccccc2-c2ccc(N(c3cccc(-c4ccccc4)c3)c3ccc4c5c(cccc35)-c3sc(-c5ccccc5)nc3-4)cc21 ; CC1(C)c2ccccc2-c2ccc(N(c3ccccc3-c3ccccc3)c3ccc4c5c(cccc35)-c3sc(-c5ccccc5)nc3-4)cc21]

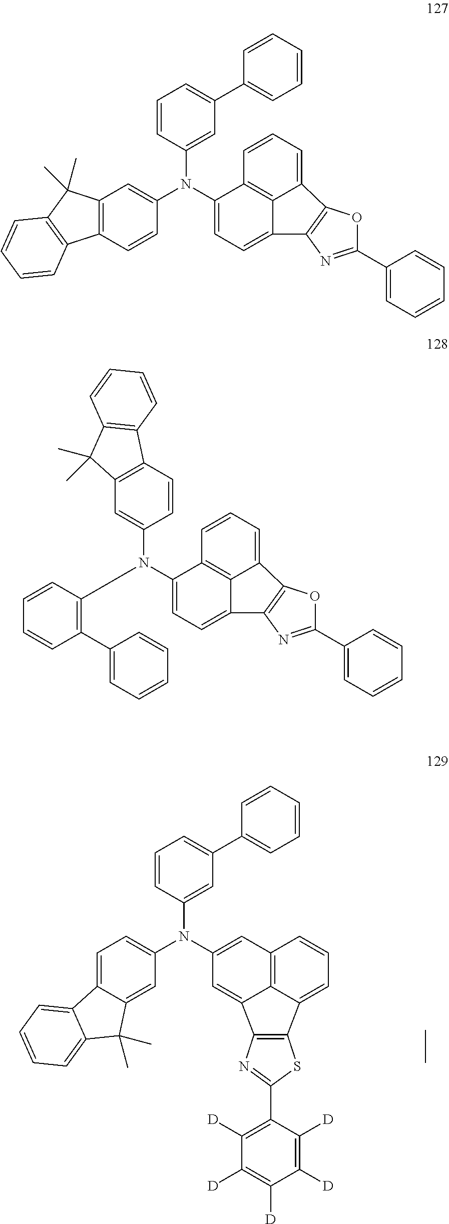

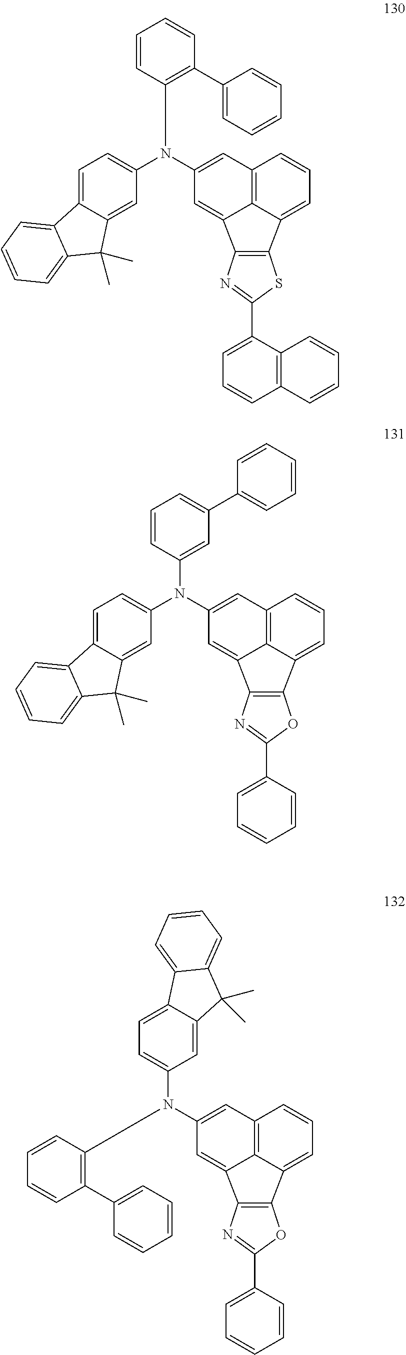

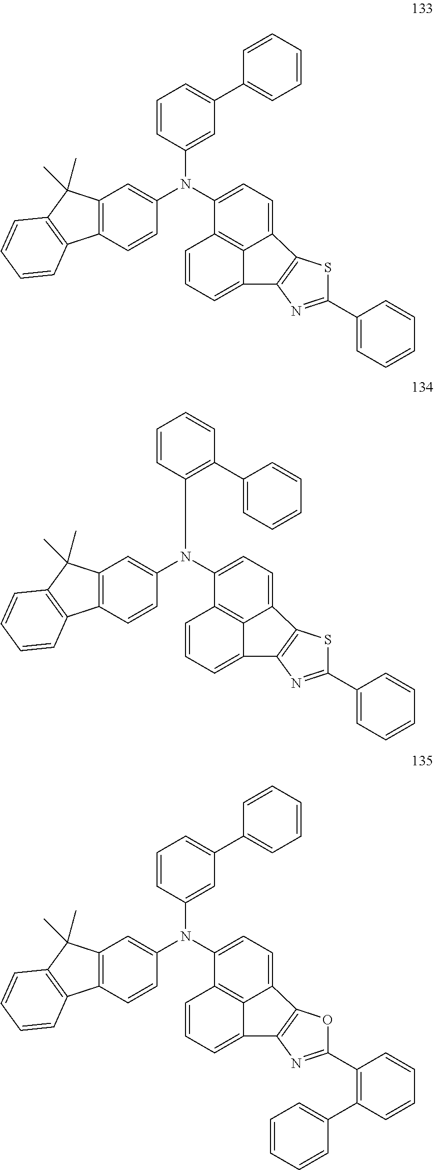 [SMILES: CC1(C)c2ccccc2-c2ccc(N(c3cccc(-c4ccccc4)c3)c3ccc4c5c(cccc35)-c3nc(-c5ccccc5)sc3-4)cc21 ; CC1(C)c2ccccc2-c2ccc(N(c3ccccc3-c3ccccc3)c3ccc4c5c(cccc35)-c3nc(-c5ccccc5)sc3-4)cc21 ; CC1(C)c2ccccc2-c2ccc(N(c3cccc(-c4ccccc4)c3)c3ccc4c5c(cccc35)-c3nc(-c5ccccc5-c5ccccc5)oc3-4)cc21]

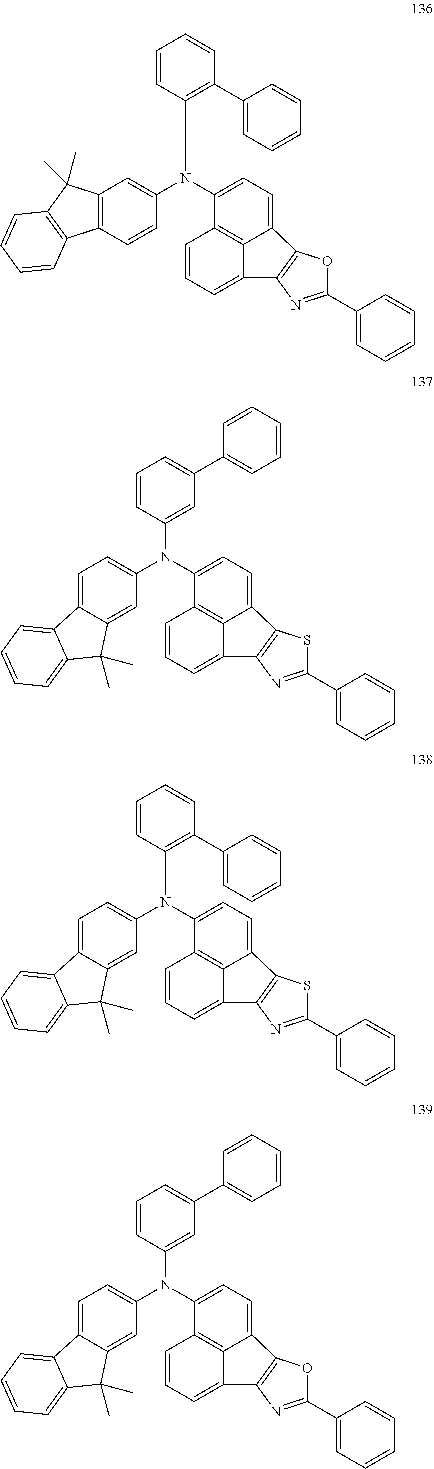 [SMILES: CC1(C)c2ccccc2-c2ccc(N(c3ccccc3-c3ccccc3)c3ccc4c5c(cccc35)-c3nc(-c5ccccc5)oc3-4)cc21 ; CC1(C)c2ccccc2-c2ccc(N(c3cccc(-c4ccccc4)c3)c3ccc4c5c(cccc35)-c3nc(-c5ccccc5)sc3-4)cc21 ; CC1(C)c2ccccc2-c2ccc(N(c3ccccc3-c3ccccc3)c3ccc4c5c(cccc35)-c3nc(-c5ccccc5)sc3-4)cc21 ; CC1(C)c2ccccc2-c2ccc(N(c3cccc(-c4ccccc4)c3)c3ccc4c5c(cccc35)-c3nc(-c5ccccc5)oc3-4)cc21]

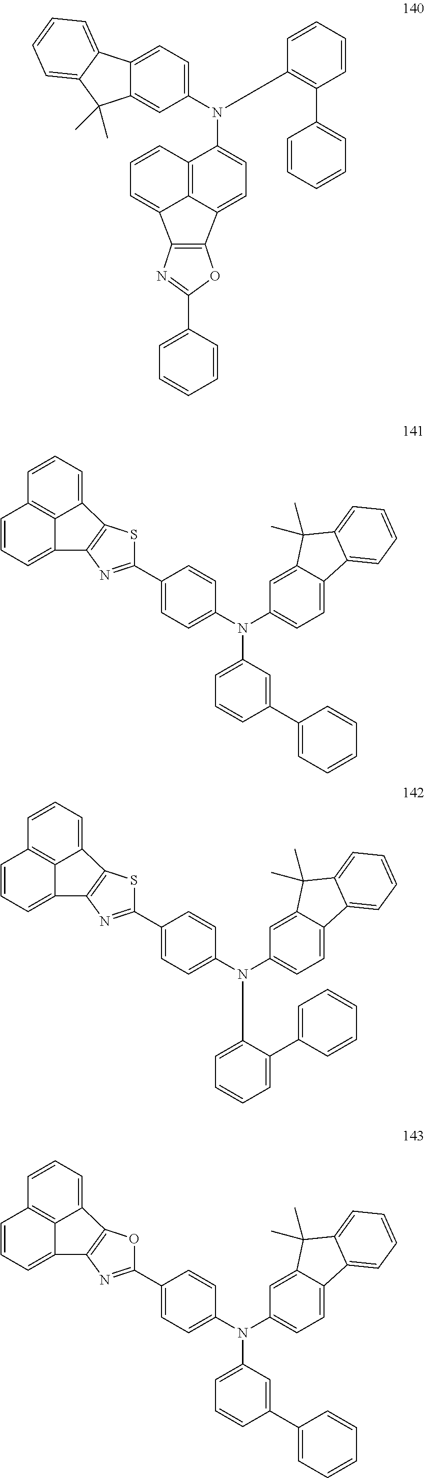 [SMILES: CC1(C)c2ccccc2-c2ccc(N(c3ccccc3-c3ccccc3)c3ccc4c5c(cccc35)-c3nc(-c5ccccc5)oc3-4)cc21 ; CC1(C)c2ccccc2-c2ccc(N(c3ccc(-c4nc5c(s4)-c4cccc6cccc-5c46)cc3)c3cccc(-c4ccccc4)c3)cc21 ; CC1(C)c2ccccc2-c2ccc(N(c3ccc(-c4nc5c(s4)-c4cccc6cccc-5c46)cc3)c3ccccc3-c3ccccc3)cc21 ; CC1(C)c2ccccc2-c2ccc(N(c3ccc(-c4nc5c(o4)-c4cccc6cccc-5c46)cc3)c3cccc(-c4ccccc4)c3)cc21]

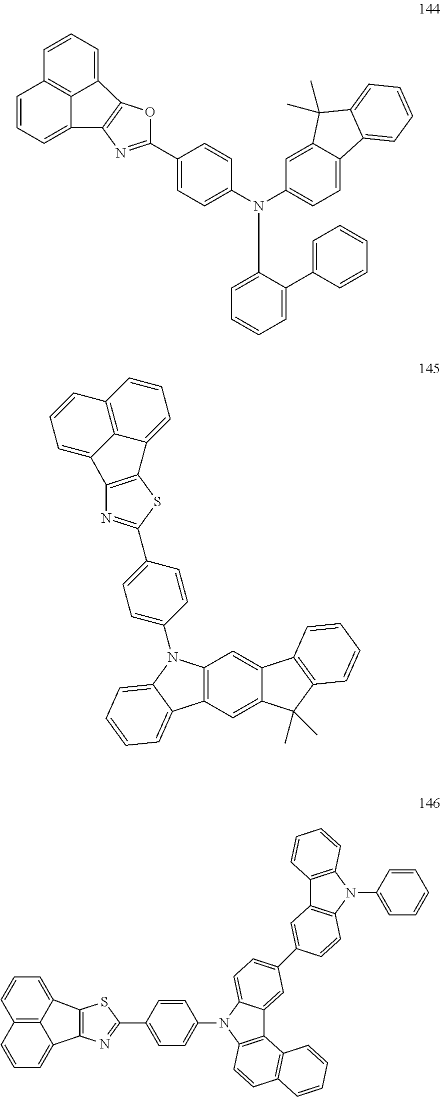 [SMILES: CC1(C)c2ccccc2-c2ccc(N(c3ccc(-c4nc5c(o4)-c4cccc6cccc-5c46)cc3)c3ccccc3-c3ccccc3)cc21 ; CC1(C)c2ccccc2-c2cc3c(cc21)c1ccccc1n3-c1ccc(-c2nc3c(s2)-c2cccc4cccc-3c24)cc1 ; c1ccc(-n2c3ccccc3c3cc(-c4ccc5c(c4)c4c6ccccc6ccc4n5-c4ccc(-c5nc6c(s5)-c5cccc7cccc-6c57)cc4)ccc32)cc1]

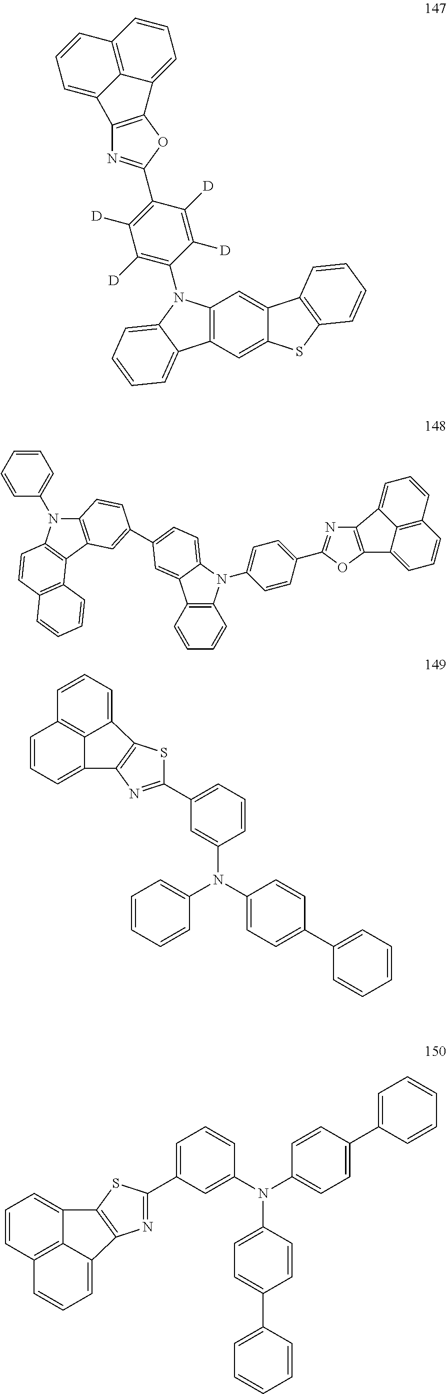

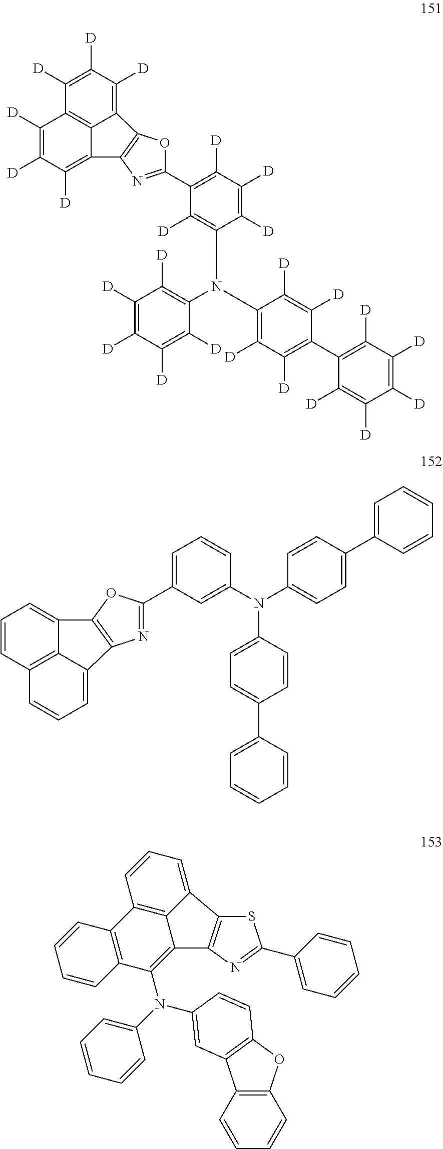 [SMILES: [2H]c1c([2H])c([2H])c(-c2c([2H])c([2H])c(N(c3c([2H])c([2H])c([2H])c([2H])c3[2H])c3c([2H])c([2H])c([2H])c(-c4nc5c(o4)-c4c([2H])c([2H])c([2H])c6c([2H])c([2H])c([2H])c-5c46)c3[2H])c([2H])c2[2H])c([2H])c1[2H] ; c1ccc(-c2ccc(N(c3ccc(-c4ccccc4)cc3)c3cccc(-c4nc5c(o4)-c4cccc6cccc-5c46)c3)cc2)cc1 ; c1ccc(-c2nc3c(s2)-c2cccc4c2c-3c(N(c2ccccc2)c2ccc3oc5ccccc5c3c2)c2ccccc24)cc1]

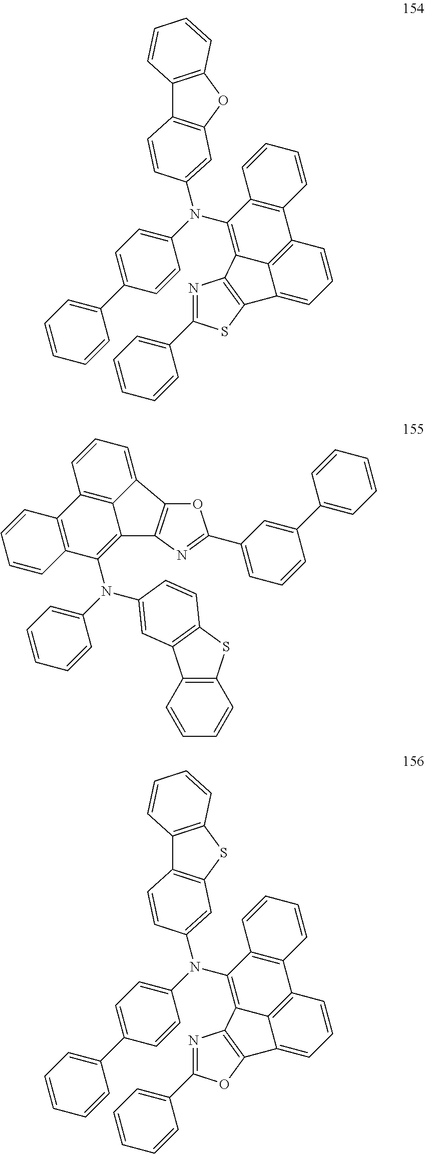

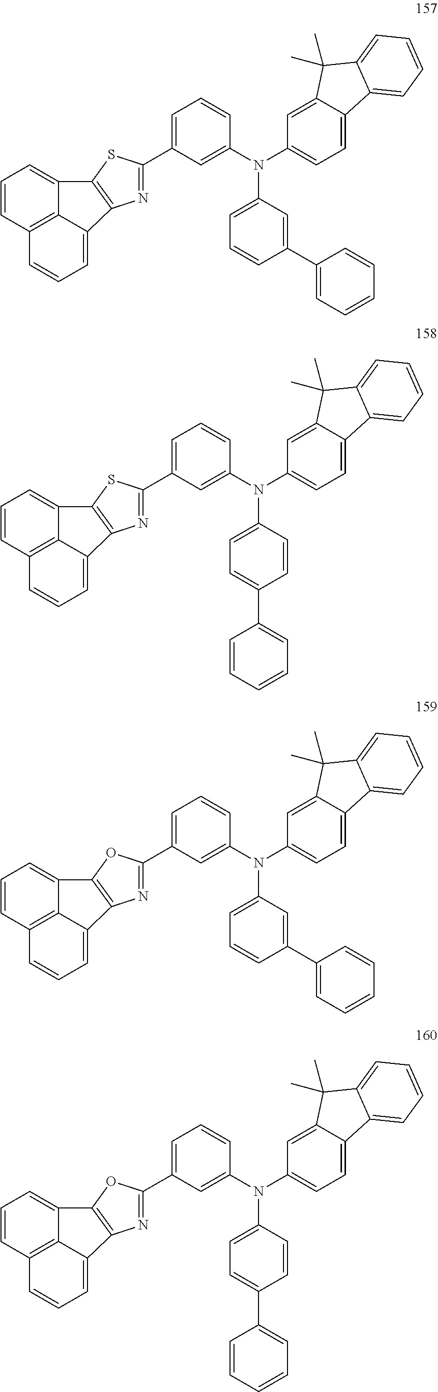 [SMILES: CC1(C)c2ccccc2-c2ccc(N(c3cccc(-c4ccccc4)c3)c3cccc(-c4nc5c(s4)-c4cccc6cccc-5c46)c3)cc21 ; CC1(C)c2ccccc2-c2ccc(N(c3ccc(-c4ccccc4)cc3)c3cccc(-c4nc5c(s4)-c4cccc6cccc-5c46)c3)cc21 ; CC1(C)c2ccccc2-c2ccc(N(c3cccc(-c4ccccc4)c3)c3cccc(-c4nc5c(o4)-c4cccc6cccc-5c46)c3)cc21 ; CC1(C)c2ccccc2-c2ccc(N(c3ccc(-c4ccccc4)cc3)c3cccc(-c4nc5c(o4)-c4cccc6cccc-5c46)c3)cc21]

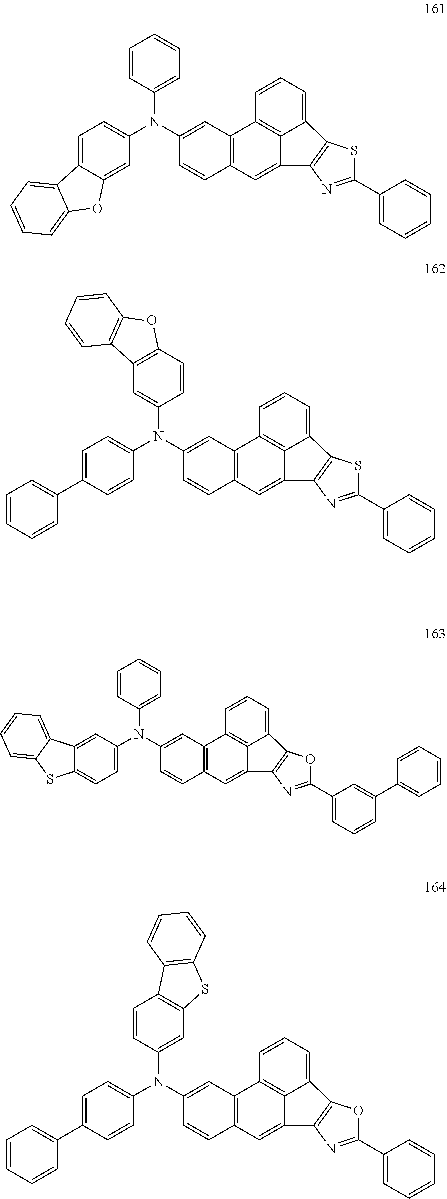 [SMILES: c1ccc(-c2nc3c(s2)-c2cccc4c2c-3cc2ccc(N(c3ccccc3)c3ccc5c(c3)oc3ccccc35)cc24)cc1 ; c1ccc(-c2ccc(N(c3ccc4cc5c6c(cccc6c4c3)-c3sc(-c4ccccc4)nc3-5)c3ccc4oc5ccccc5c4c3)cc2)cc1 ; c1ccc(-c2cccc(-c3nc4c(o3)-c3cccc5c3c-4cc3ccc(N(c4ccccc4)c4ccc6sc7ccccc7c6c4)cc35)c2)cc1 ; c1ccc(-c2ccc(N(c3ccc4c(c3)sc3ccccc34)c3ccc4cc5c6c(cccc6c4c3)-c3oc(-c4ccccc4)nc3-5)cc2)cc1]

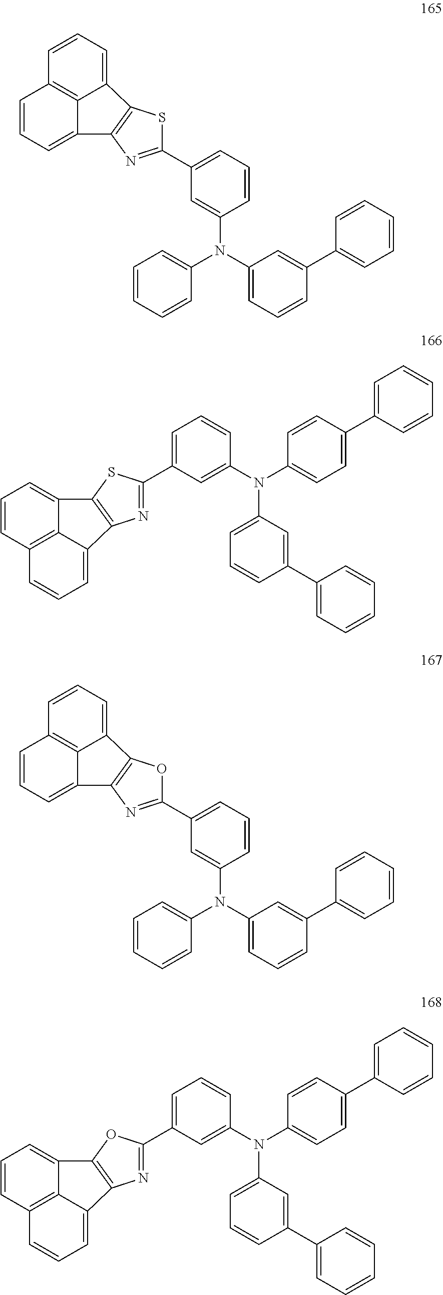 [SMILES: c1ccc(-c2cccc(N(c3ccccc3)c3cccc(-c4nc5c(s4)-c4cccc6cccc-5c46)c3)c2)cc1 ; c1ccc(-c2ccc(N(c3cccc(-c4ccccc4)c3)c3cccc(-c4nc5c(s4)-c4cccc6cccc-5c46)c3)cc2)cc1 ; c1ccc(-c2cccc(N(c3ccccc3)c3cccc(-c4nc5c(o4)-c4cccc6cccc-5c46)c3)c2)cc1 ; c1ccc(-c2ccc(N(c3cccc(-c4ccccc4)c3)c3cccc(-c4nc5c(o4)-c4cccc6cccc-5c46)c3)cc2)cc1]

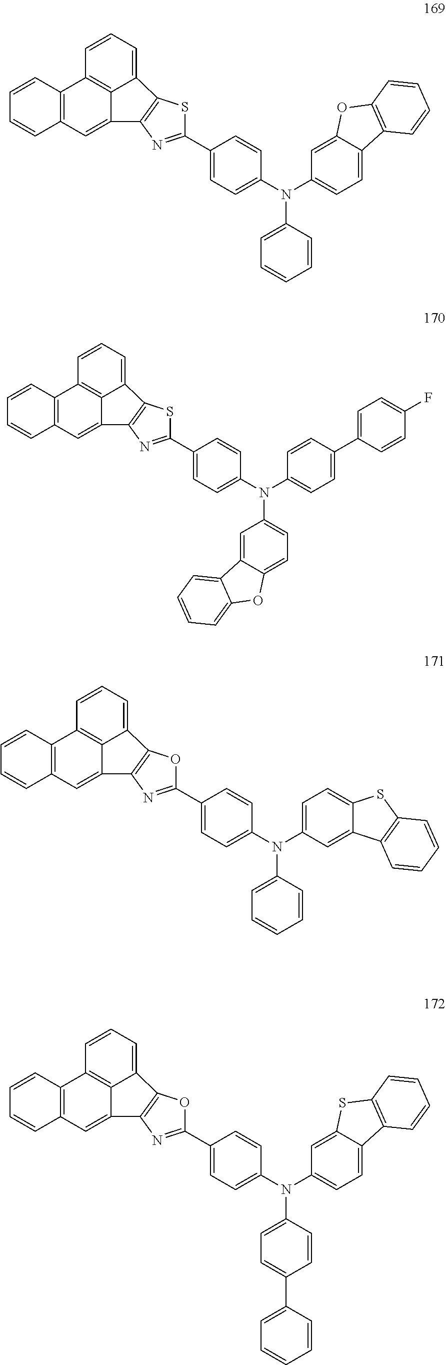 [SMILES: c1ccc(N(c2ccc(-c3nc4c(s3)-c3cccc5c3c-4cc3ccccc35)cc2)c2ccc3c(c2)oc2ccccc23)cc1 ; Fc1ccc(-c2ccc(N(c3ccc(-c4nc5c(s4)-c4cccc6c4c-5cc4ccccc46)cc3)c3ccc4oc5ccccc5c4c3)cc2)cc1 ; c1ccc(N(c2ccc(-c3nc4c(o3)-c3cccc5c3c-4cc3ccccc35)cc2)c2ccc3sc4ccccc4c3c2)cc1 ; c1ccc(-c2ccc(N(c3ccc(-c4nc5c(o4)-c4cccc6c4c-5cc4ccccc46)cc3)c3ccc4c(c3)sc3ccccc34)cc2)cc1]

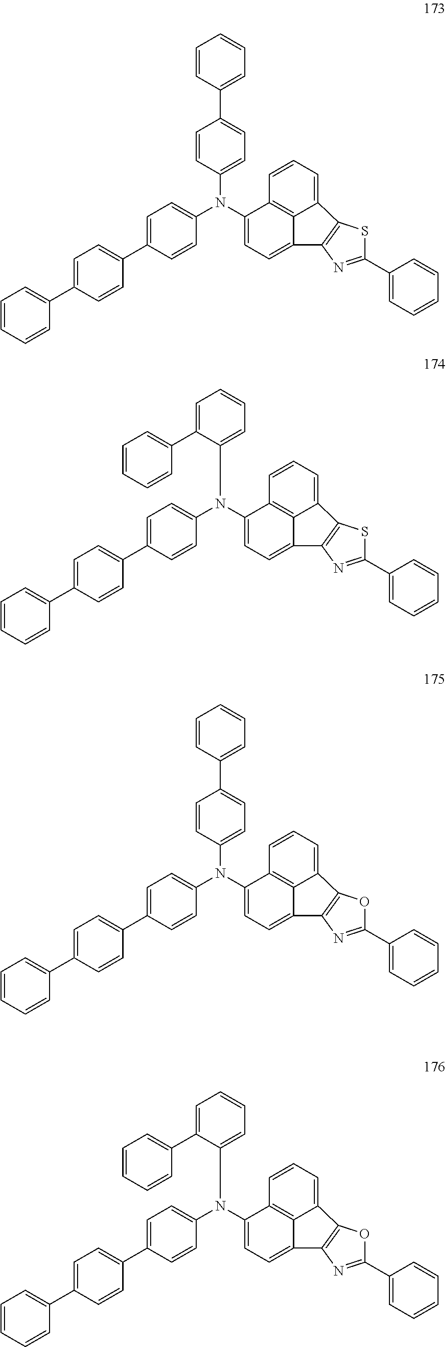 [SMILES: c1ccc(-c2ccc(-c3ccc(N(c4ccc(-c5ccccc5)cc4)c4ccc5c6c(cccc46)-c4sc(-c6ccccc6)nc4-5)cc3)cc2)cc1 ; c1ccc(-c2ccc(-c3ccc(N(c4ccccc4-c4ccccc4)c4ccc5c6c(cccc46)-c4sc(-c6ccccc6)nc4-5)cc3)cc2)cc1 ; c1ccc(-c2ccc(-c3ccc(N(c4ccc(-c5ccccc5)cc4)c4ccc5c6c(cccc46)-c4oc(-c6ccccc6)nc4-5)cc3)cc2)cc1 ; c1ccc(-c2ccc(-c3ccc(N(c4ccccc4-c4ccccc4)c4ccc5c6c(cccc46)-c4oc(-c6ccccc6)nc4-5)cc3)cc2)cc1]

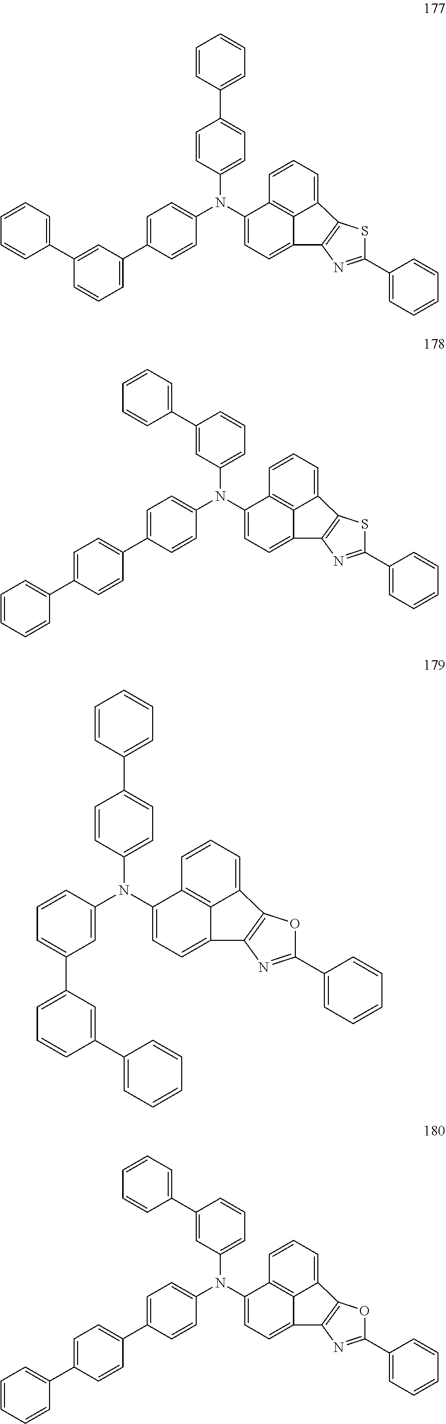 [SMILES: c1ccc(-c2ccc(N(c3ccc(-c4cccc(-c5ccccc5)c4)cc3)c3ccc4c5c(cccc35)-c3sc(-c5ccccc5)nc3-4)cc2)cc1 ; c1ccc(-c2ccc(-c3ccc(N(c4cccc(-c5ccccc5)c4)c4ccc5c6c(cccc46)-c4sc(-c6ccccc6)nc4-5)cc3)cc2)cc1 ; c1ccc(-c2ccc(N(c3cccc(-c4cccc(-c5ccccc5)c4)c3)c3ccc4c5c(cccc35)-c3oc(-c5ccccc5)nc3-4)cc2)cc1 ; c1ccc(-c2ccc(-c3ccc(N(c4cccc(-c5ccccc5)c4)c4ccc5c6c(cccc46)-c4oc(-c6ccccc6)nc4-5)cc3)cc2)cc1]

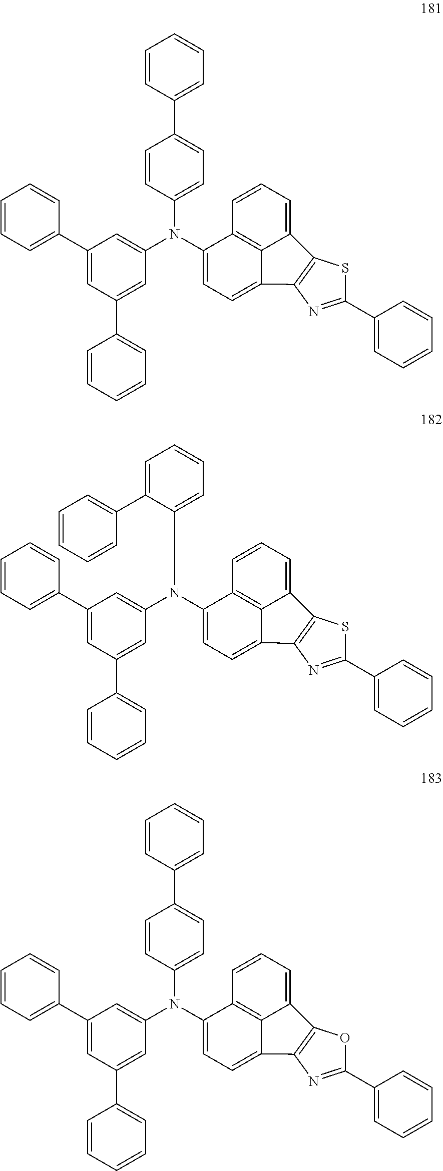 [SMILES: c1ccc(-c2ccc(N(c3cc(-c4ccccc4)cc(-c4ccccc4)c3)c3ccc4c5c(cccc35)-c3sc(-c5ccccc5)nc3-4)cc2)cc1 ; c1ccc(-c2cc(-c3ccccc3)cc(N(c3ccccc3-c3ccccc3)c3ccc4c5c(cccc35)-c3sc(-c5ccccc5)nc3-4)c2)cc1 ; c1ccc(-c2ccc(N(c3cc(-c4ccccc4)cc(-c4ccccc4)c3)c3ccc4c5c(cccc35)-c3oc(-c5ccccc5)nc3-4)cc2)cc1]

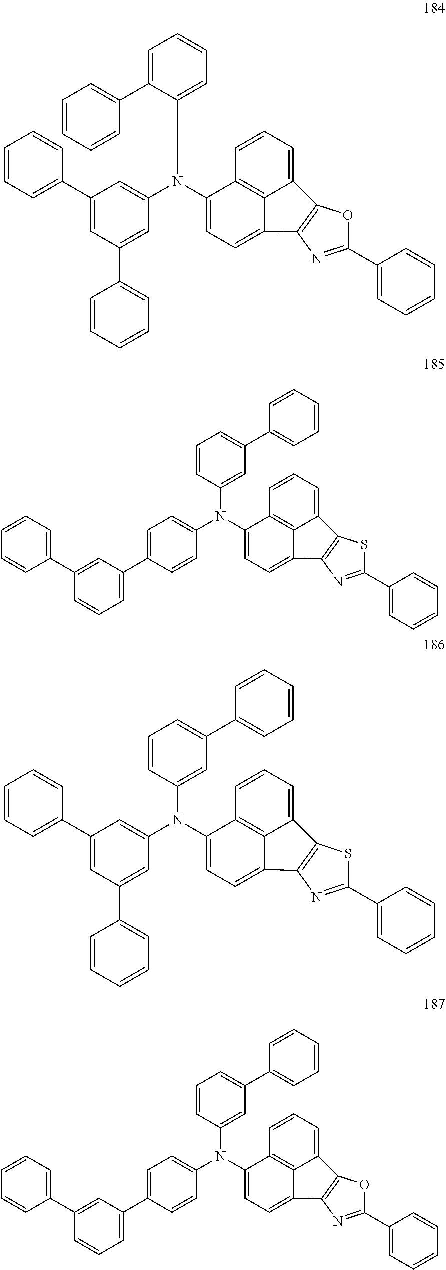 [SMILES: c1ccc(-c2cc(-c3ccccc3)cc(N(c3ccccc3-c3ccccc3)c3ccc4c5c(cccc35)-c3oc(-c5ccccc5)nc3-4)c2)cc1 ; c1ccc(-c2cccc(-c3ccc(N(c4cccc(-c5ccccc5)c4)c4ccc5c6c(cccc46)-c4sc(-c6ccccc6)nc4-5)cc3)c2)cc1 ; c1ccc(-c2cccc(N(c3cc(-c4ccccc4)cc(-c4ccccc4)c3)c3ccc4c5c(cccc35)-c3sc(-c5ccccc5)nc3-4)c2)cc1 ; c1ccc(-c2cccc(-c3ccc(N(c4cccc(-c5ccccc5)c4)c4ccc5c6c(cccc46)-c4oc(-c6ccccc6)nc4-5)cc3)c2)cc1]

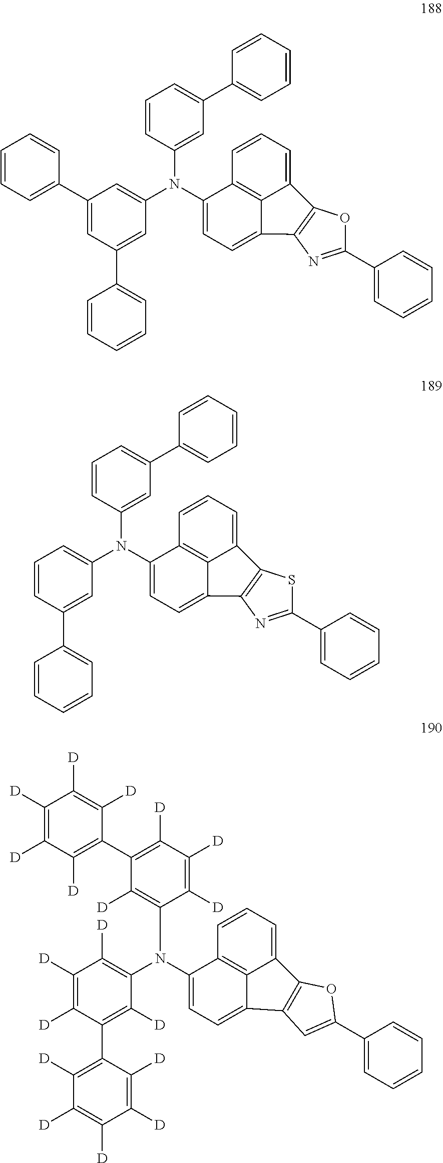

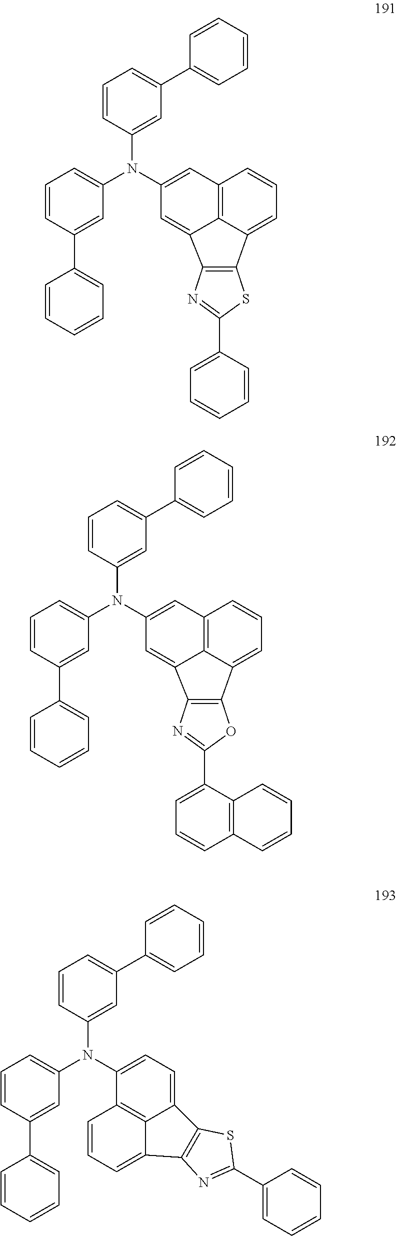

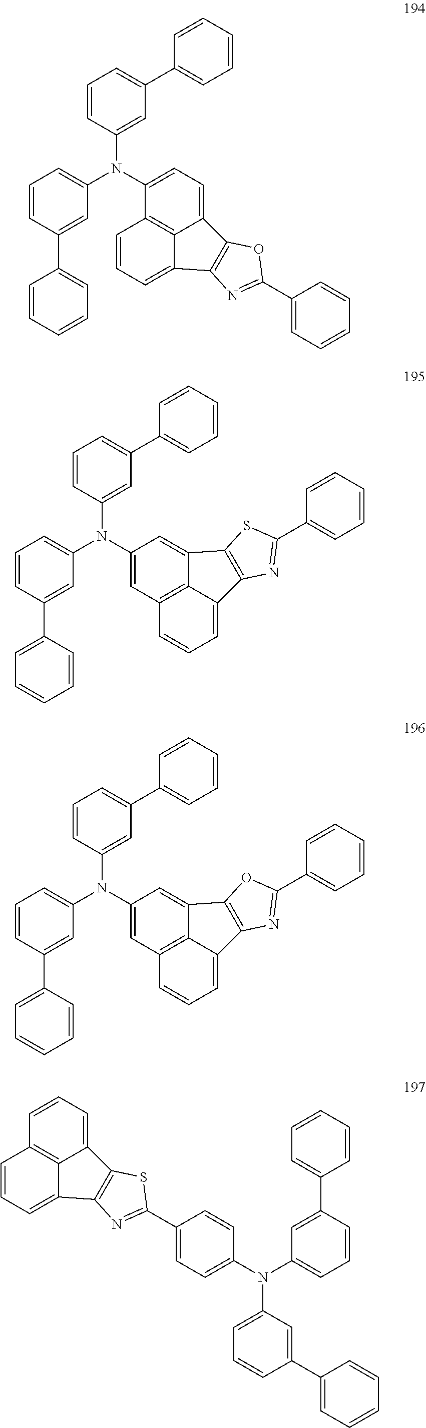

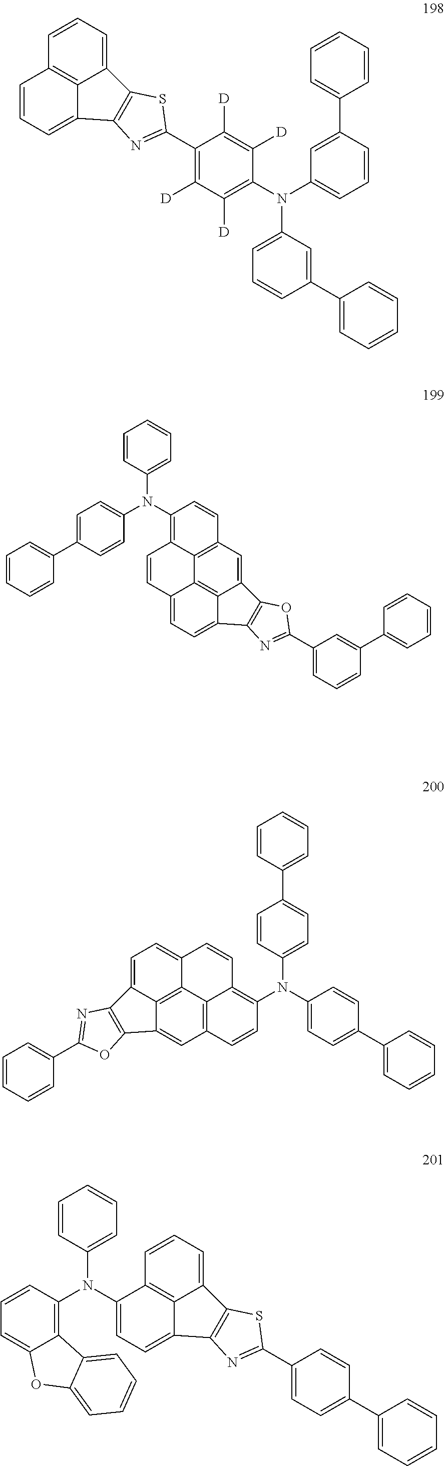 [SMILES: [2H]c1c([2H])c(N(c2cccc(-c3ccccc3)c2)c2cccc(-c3ccccc3)c2)c([2H])c([2H])c1-c1nc2c(s1)-c1cccc3cccc-2c13 ; c1ccc(-c2ccc(N(c3ccccc3)c3ccc4cc5c6c(ccc7ccc3c4c76)-c3nc(-c4cccc(-c6ccccc6)c4)oc3-5)cc2)cc1 ; c1ccc(-c2ccc(N(c3ccc(-c4ccccc4)cc3)c3ccc4cc5c6c(ccc7ccc3c4c76)-c3nc(-c4ccccc4)oc3-5)cc2)cc1 ; c1ccc(-c2ccc(-c3nc4c(s3)-c3cccc5c(N(c6ccccc6)c6cccc7oc8ccccc8c67)ccc-4c35)cc2)cc1]

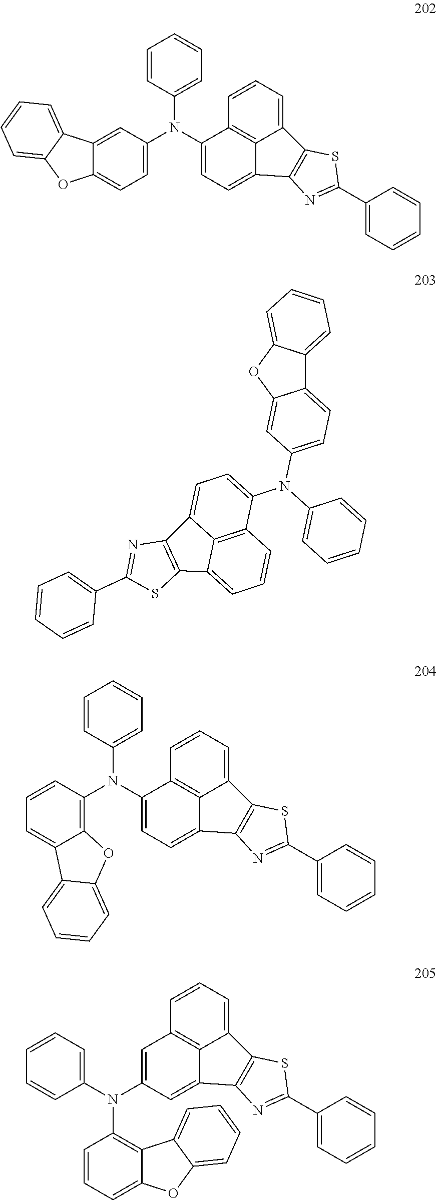 [SMILES: c1ccc(-c2nc3c(s2)-c2cccc4c(N(c5ccccc5)c5ccc6oc7ccccc7c6c5)ccc-3c24)cc1 ; c1ccc(-c2nc3c(s2)-c2cccc4c(N(c5ccccc5)c5ccc6c(c5)oc5ccccc56)ccc-3c24)cc1 ; c1ccc(-c2nc3c(s2)-c2cccc4c(N(c5ccccc5)c5cccc6c5oc5ccccc56)ccc-3c24)cc1 ; c1ccc(-c2nc3c(s2)-c2cccc4cc(N(c5ccccc5)c5cccc6oc7ccccc7c56)cc-3c24)cc1]

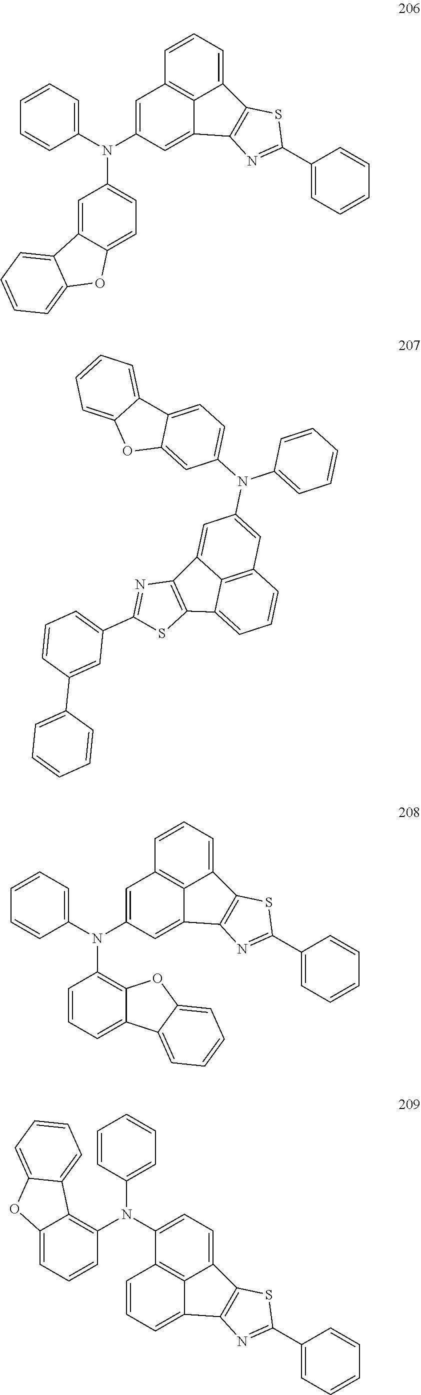 [SMILES: c1ccc(-c2nc3c(s2)-c2cccc4cc(N(c5ccccc5)c5ccc6oc7ccccc7c6c5)cc-3c24)cc1 ; c1ccc(-c2cccc(-c3nc4c(s3)-c3cccc5cc(N(c6ccccc6)c6ccc7c(c6)oc6ccccc67)cc-4c35)c2)cc1 ; c1ccc(-c2nc3c(s2)-c2cccc4cc(N(c5ccccc5)c5cccc6c5oc5ccccc56)cc-3c24)cc1 ; c1ccc(-c2nc3c(s2)-c2ccc(N(c4ccccc4)c4cccc5oc6ccccc6c45)c4cccc-3c24)cc1]

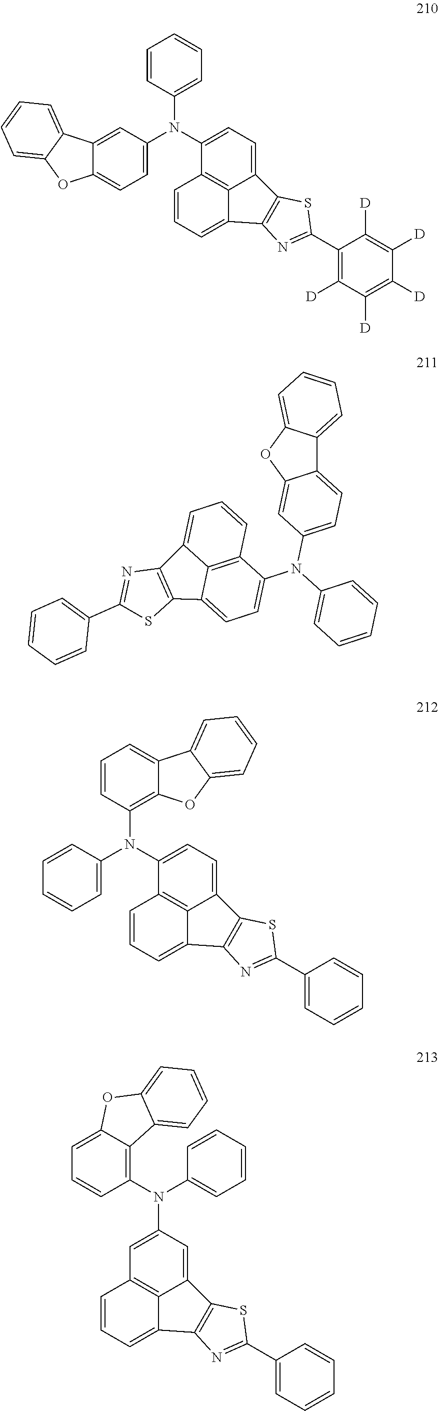 [SMILES: [2H]c1c([2H])c([2H])c(-c2nc3c(s2)-c2ccc(N(c4ccccc4)c4ccc5oc6ccccc6c5c4)c4cccc-3c24)c([2H])c1[2H] ; c1ccc(-c2nc3c(s2)-c2ccc(N(c4ccccc4)c4ccc5c(c4)oc4ccccc45)c4cccc-3c24)cc1 ; c1ccc(-c2nc3c(s2)-c2ccc(N(c4ccccc4)c4cccc5c4oc4ccccc45)c4cccc-3c24)cc1 ; c1ccc(-c2nc3c(s2)-c2cc(N(c4ccccc4)c4cccc5oc6ccccc6c45)cc4cccc-3c24)cc1]

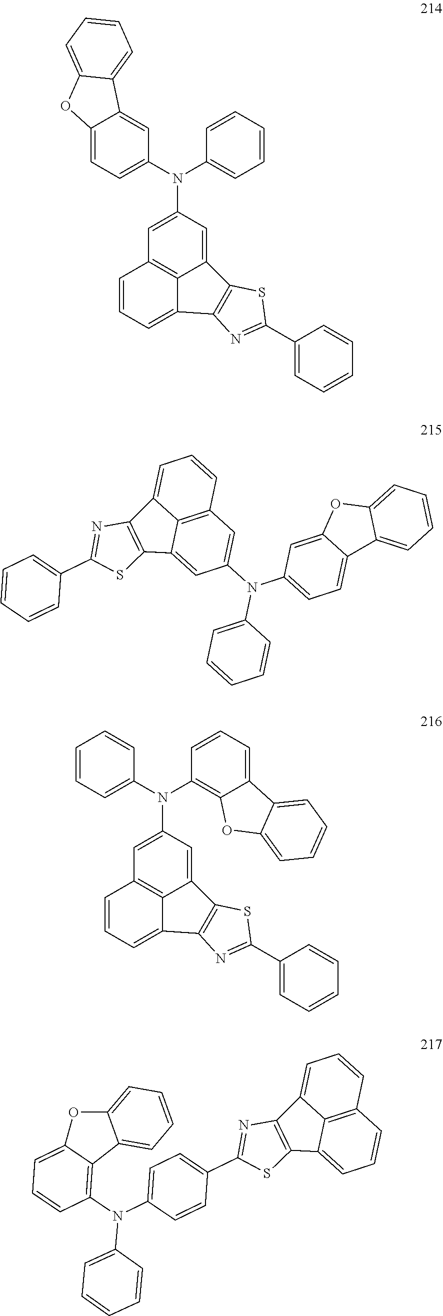 [SMILES: c1ccc(-c2nc3c(s2)-c2cc(N(c4ccccc4)c4ccc5oc6ccccc6c5c4)cc4cccc-3c24)cc1 ; c1ccc(-c2nc3c(s2)-c2cc(N(c4ccccc4)c4ccc5c(c4)oc4ccccc45)cc4cccc-3c24)cc1 ; c1ccc(-c2nc3c(s2)-c2cc(N(c4ccccc4)c4cccc5c4oc4ccccc45)cc4cccc-3c24)cc1 ; c1ccc(N(c2ccc(-c3nc4c(s3)-c3cccc5cccc-4c35)cc2)c2cccc3oc4ccccc4c23)cc1]

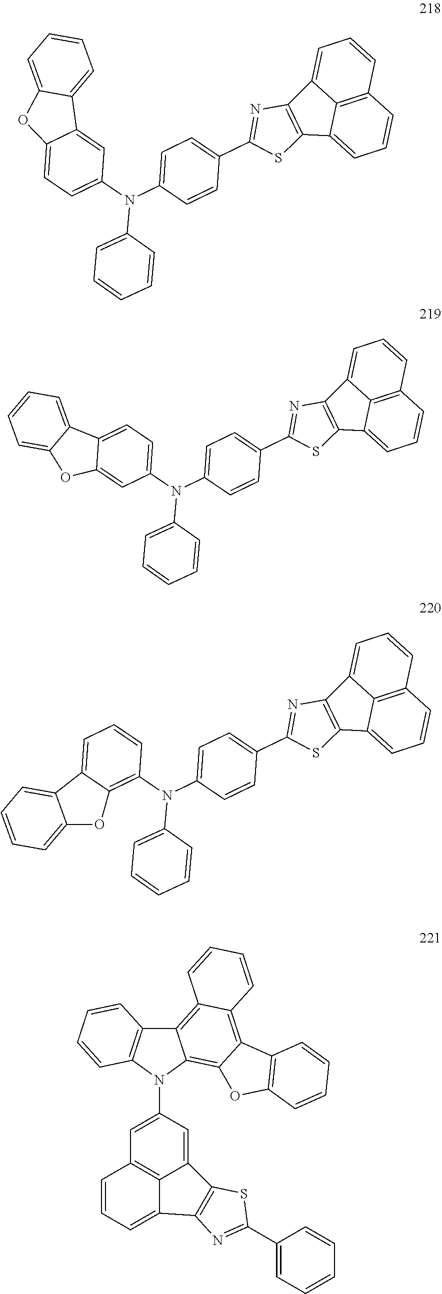 [SMILES: c1ccc(N(c2ccc(-c3nc4c(s3)-c3cccc5cccc-4c35)cc2)c2ccc3oc4ccccc4c3c2)cc1 ; c1ccc(N(c2ccc(-c3nc4c(s3)-c3cccc5cccc-4c35)cc2)c2ccc3c(c2)oc2ccccc23)cc1 ; c1ccc(N(c2ccc(-c3nc4c(s3)-c3cccc5cccc-4c35)cc2)c2cccc3c2oc2ccccc23)cc1 ; c1ccc(-c2nc3c(s2)-c2cc(-n4c5ccccc5c5c6ccccc6c6c7ccccc7oc6c54)cc4cccc-3c24)cc1]

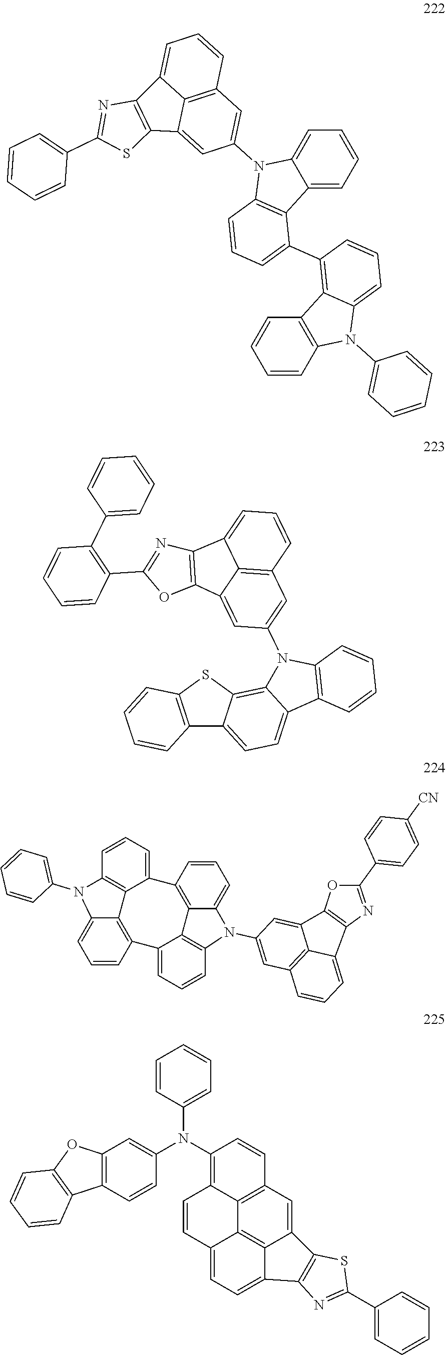 [SMILES: c1ccc(-c2nc3c(s2)-c2cc(-n4c5ccccc5c5c(-c6cccc7c6c6ccccc6n7-c6ccccc6)cccc54)cc4cccc-3c24)cc1 ; c1ccc(-c2ccccc2-c2nc3c(o2)-c2cc(-n4c5ccccc5c5ccc6c7ccccc7sc6c54)cc4cccc-3c24)cc1 ; N#Cc1ccc(-c2nc3c(o2)-c2cc(-n4c5cccc6c7cccc8c7c7c(cccc7n8-c7ccccc7)c7cccc4c7c65)cc4cccc-3c24)cc1 ; c1ccc(-c2nc3c(s2)-c2cc4ccc(N(c5ccccc5)c5ccc6c(c5)oc5ccccc56)c5ccc6ccc-3c2c6c45)cc1]

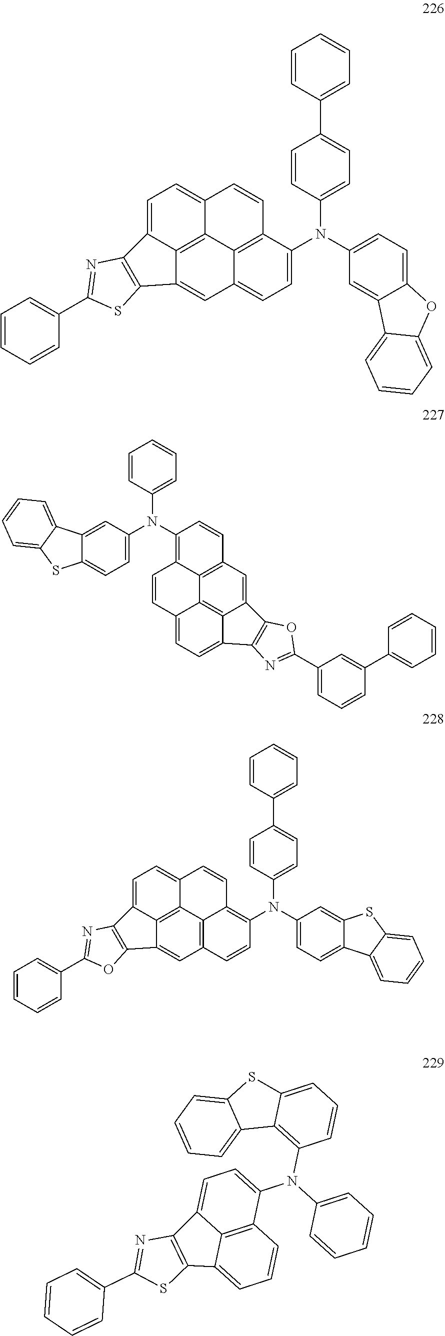 [SMILES: c1ccc(-c2ccc(N(c3ccc4oc5ccccc5c4c3)c3ccc4cc5c6c(ccc7ccc3c4c76)-c3nc(-c4ccccc4)sc3-5)cc2)cc1 ; c1ccc(-c2cccc(-c3nc4c(o3)-c3cc5ccc(N(c6ccccc6)c6ccc7sc8ccccc8c7c6)c6ccc7ccc-4c3c7c56)c2)cc1 ; c1ccc(-c2ccc(N(c3ccc4c(c3)sc3ccccc34)c3ccc4cc5c6c(ccc7ccc3c4c76)-c3nc(-c4ccccc4)oc3-5)cc2)cc1 ; c1ccc(-c2nc3c(s2)-c2cccc4c(N(c5ccccc5)c5cccc6sc7ccccc7c56)ccc-3c24)cc1]

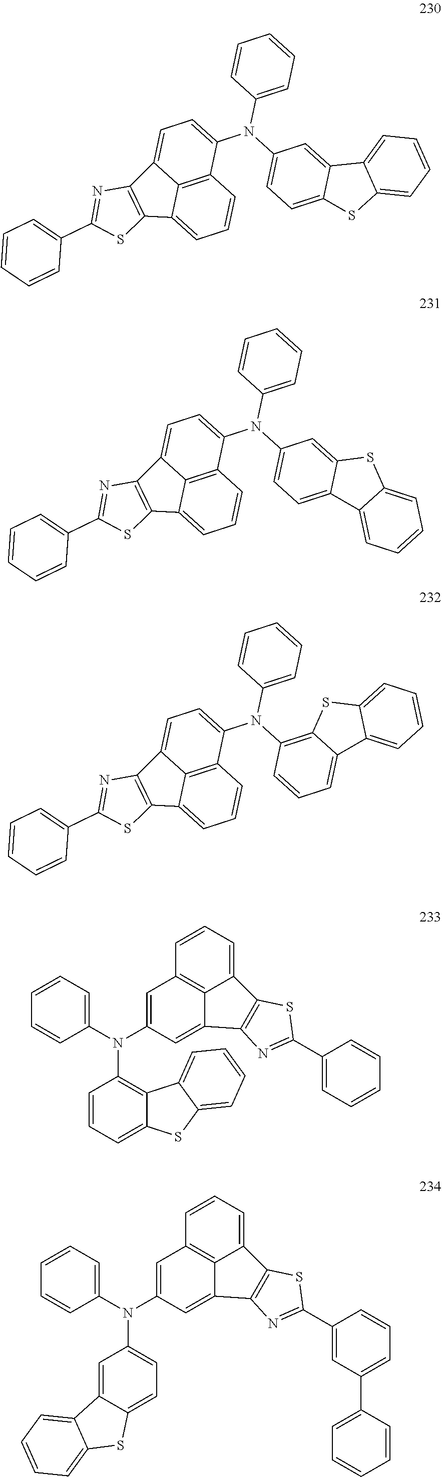 [SMILES: c1ccc(-c2nc3c(s2)-c2cccc4c(N(c5ccccc5)c5ccc6sc7ccccc7c6c5)ccc-3c24)cc1 ; c1ccc(-c2nc3c(s2)-c2cccc4c(N(c5ccccc5)c5ccc6c(c5)sc5ccccc56)ccc-3c24)cc1 ; c1ccc(-c2nc3c(s2)-c2cccc4c(N(c5ccccc5)c5cccc6c5sc5ccccc56)ccc-3c24)cc1 ; c1ccc(-c2nc3c(s2)-c2cccc4cc(N(c5ccccc5)c5cccc6sc7ccccc7c56)cc-3c24)cc1 ; c1ccc(-c2cccc(-c3nc4c(s3)-c3cccc5cc(N(c6ccccc6)c6ccc7sc8ccccc8c7c6)cc-4c35)c2)cc1]

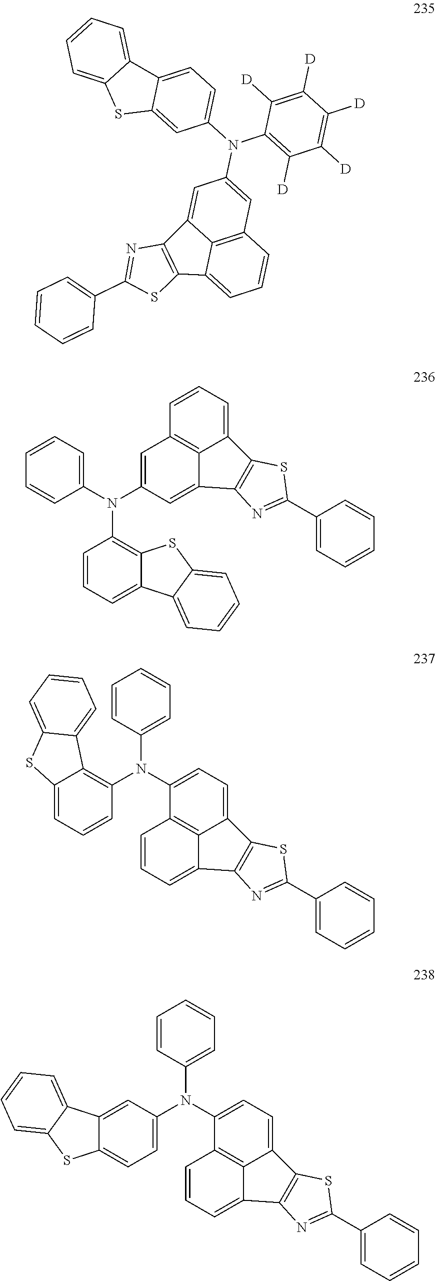 [SMILES: [2H]c1c([2H])c([2H])c(N(c2cc3c4c(cccc4c2)-c2sc(-c4ccccc4)nc2-3)c2ccc3c(c2)sc2ccccc23)c([2H])c1[2H] ; c1ccc(-c2nc3c(s2)-c2cccc4cc(N(c5ccccc5)c5cccc6c5sc5ccccc56)cc-3c24)cc1 ; c1ccc(-c2nc3c(s2)-c2ccc(N(c4ccccc4)c4cccc5sc6ccccc6c45)c4cccc-3c24)cc1 ; c1ccc(-c2nc3c(s2)-c2ccc(N(c4ccccc4)c4ccc5sc6ccccc6c5c4)c4cccc-3c24)cc1]

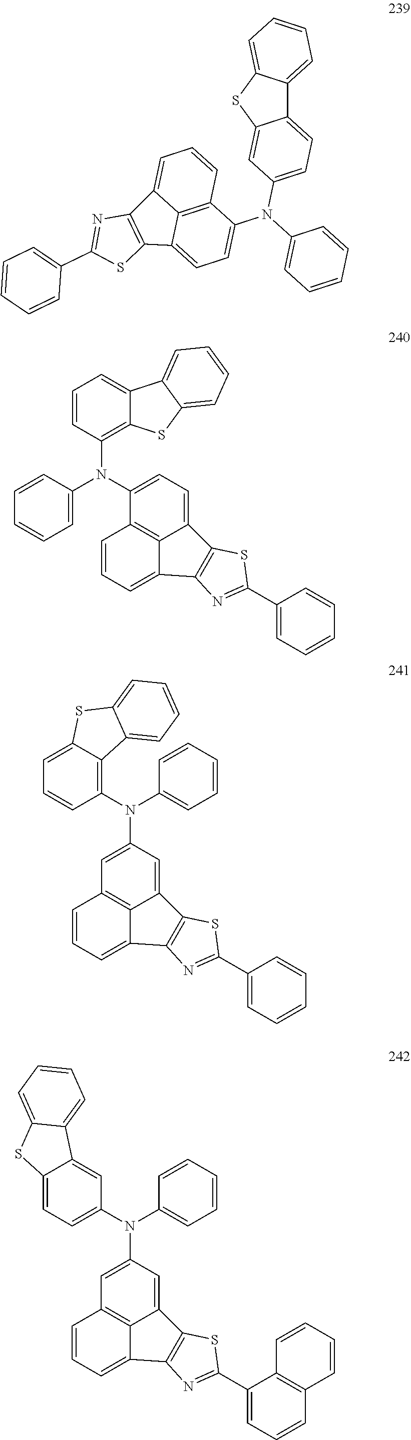 [SMILES: c1ccc(-c2nc3c(s2)-c2ccc(N(c4ccccc4)c4ccc5c(c4)sc4ccccc45)c4cccc-3c24)cc1 ; c1ccc(-c2nc3c(s2)-c2ccc(N(c4ccccc4)c4cccc5c4sc4ccccc45)c4cccc-3c24)cc1 ; c1ccc(-c2nc3c(s2)-c2cc(N(c4ccccc4)c4cccc5sc6ccccc6c45)cc4cccc-3c24)cc1 ; c1ccc(N(c2cc3c4c(cccc4c2)-c2nc(-c4cccc5ccccc45)sc2-3)c2ccc3sc4ccccc4c3c2)cc1]

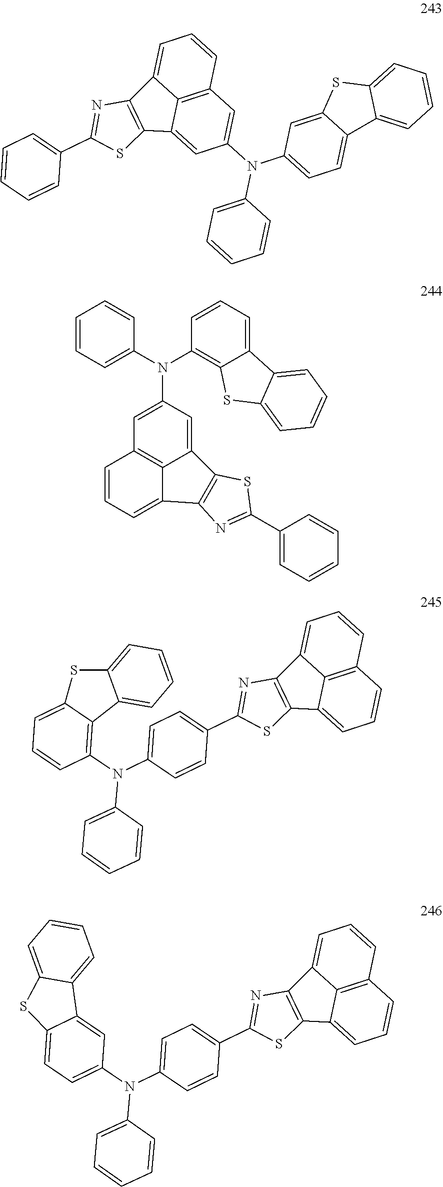

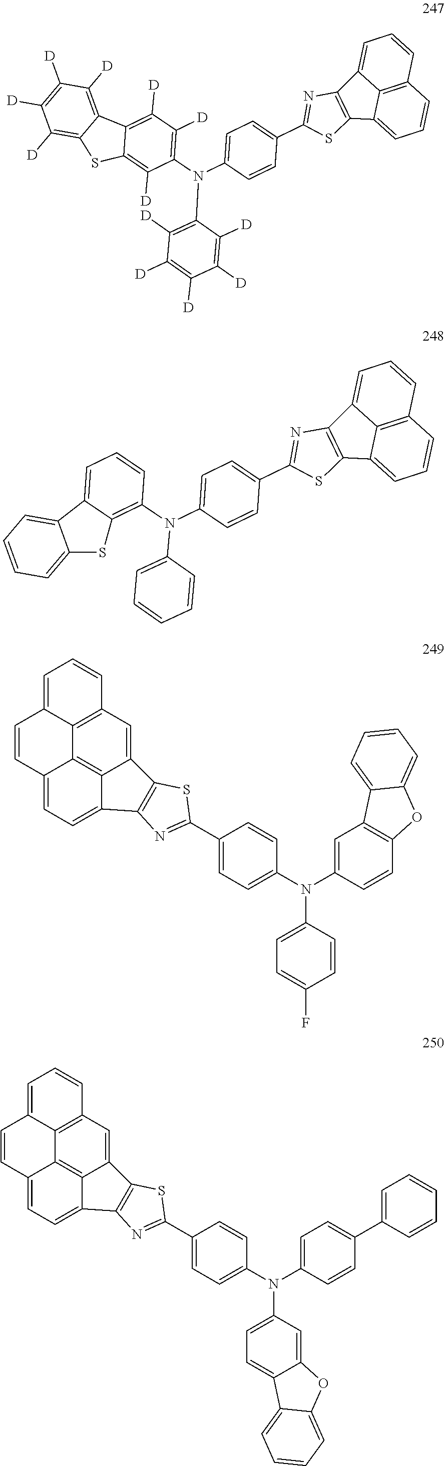 [SMILES: [2H]c1c([2H])c([2H])c(N(c2ccc(-c3nc4c(s3)-c3cccc5cccc-4c35)cc2)c2c([2H])c([2H])c3c(sc4c([2H])c([2H])c([2H])c([2H])c43)c2[2H])c([2H])c1[2H] ; c1ccc(N(c2ccc(-c3nc4c(s3)-c3cccc5cccc-4c35)cc2)c2cccc3c2sc2ccccc23)cc1 ; Fc1ccc(N(c2ccc(-c3nc4c(s3)-c3cc5cccc6ccc7ccc-4c3c7c65)cc2)c2ccc3oc4ccccc4c3c2)cc1 ; c1ccc(-c2ccc(N(c3ccc(-c4nc5c(s4)-c4cc6cccc7ccc8ccc-5c4c8c76)cc3)c3ccc4c(c3)oc3ccccc34)cc2)cc1]

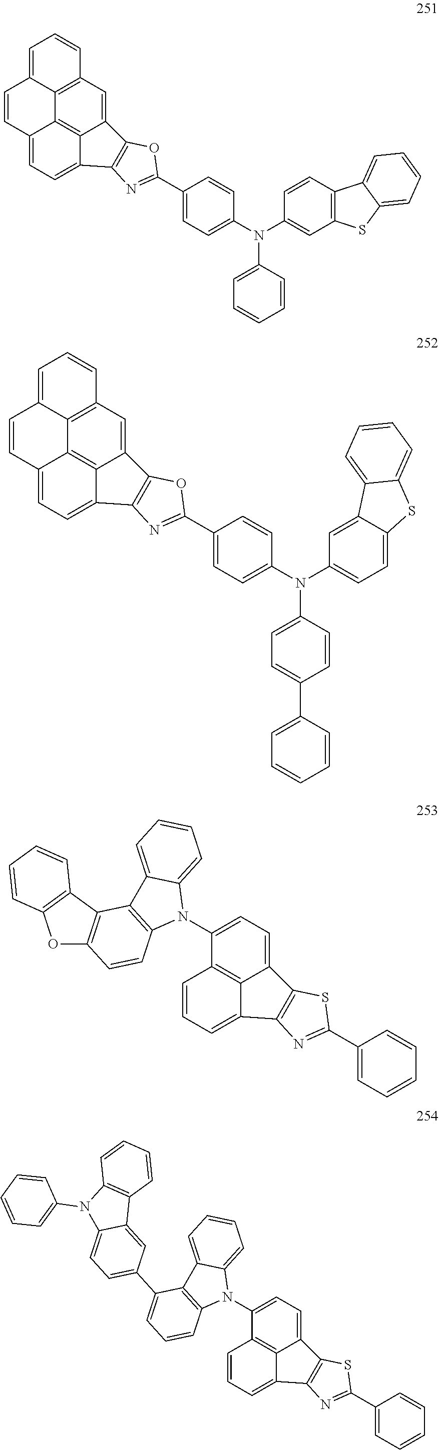 [SMILES: c1ccc(N(c2ccc(-c3nc4c(o3)-c3cc5cccc6ccc7ccc-4c3c7c65)cc2)c2ccc3c(c2)sc2ccccc23)cc1 ; c1ccc(-c2ccc(N(c3ccc(-c4nc5c(o4)-c4cc6cccc7ccc8ccc-5c4c8c76)cc3)c3ccc4sc5ccccc5c4c3)cc2)cc1 ; c1ccc(-c2nc3c(s2)-c2ccc(-n4c5ccccc5c5c6c(ccc54)oc4ccccc46)c4cccc-3c24)cc1 ; c1ccc(-c2nc3c(s2)-c2ccc(-n4c5ccccc5c5c(-c6ccc7c(c6)c6ccccc6n7-c6ccccc6)cccc54)c4cccc-3c24)cc1]

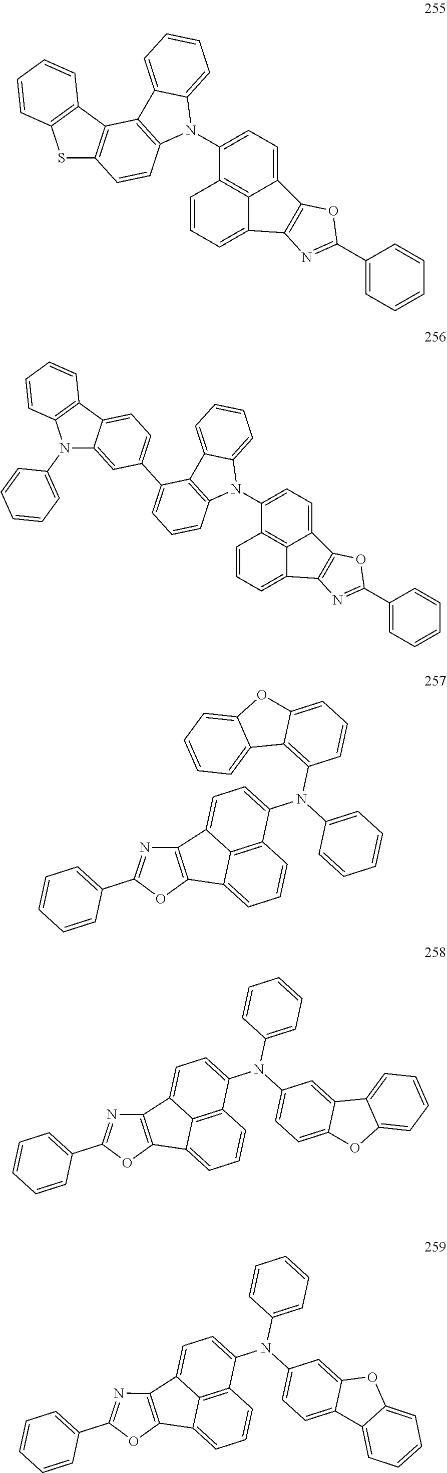 [SMILES: c1ccc(-c2nc3c(o2)-c2ccc(-n4c5ccccc5c5c6c(ccc54)sc4ccccc46)c4cccc-3c24)cc1 ; c1ccc(-c2nc3c(o2)-c2ccc(-n4c5ccccc5c5c(-c6ccc7c8ccccc8n(-c8ccccc8)c7c6)cccc54)c4cccc-3c24)cc1 ; c1ccc(-c2nc3c(o2)-c2cccc4c(N(c5ccccc5)c5cccc6oc7ccccc7c56)ccc-3c24)cc1 ; c1ccc(-c2nc3c(o2)-c2cccc4c(N(c5ccccc5)c5ccc6oc7ccccc7c6c5)ccc-3c24)cc1 ; c1ccc(-c2nc3c(o2)-c2cccc4c(N(c5ccccc5)c5ccc6c(c5)oc5ccccc56)ccc-3c24)cc1]

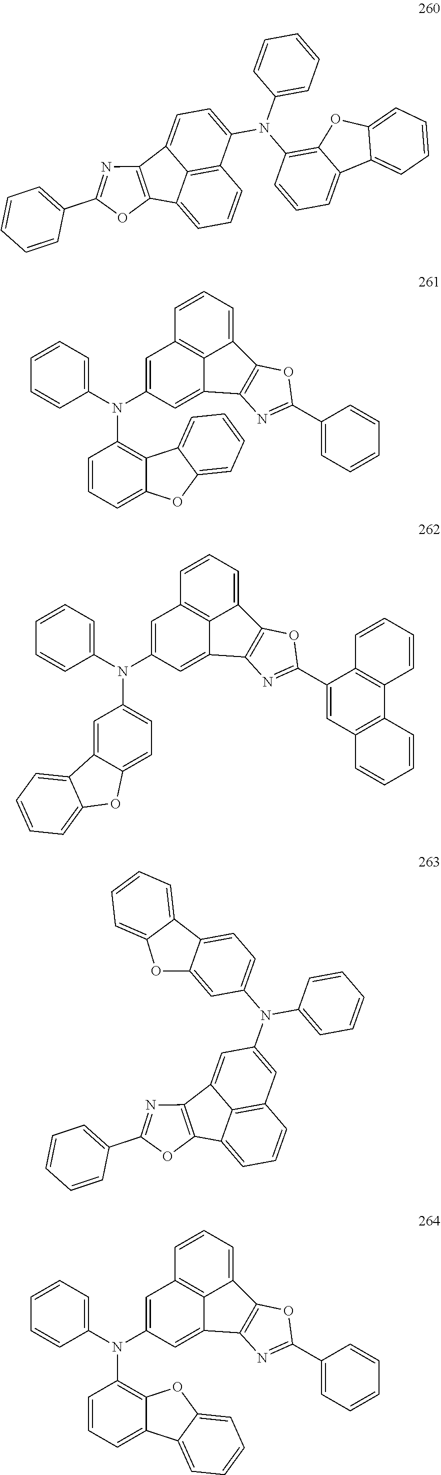 [SMILES: c1ccc(-c2nc3c(o2)-c2cccc4c(N(c5ccccc5)c5cccc6c5oc5ccccc56)ccc-3c24)cc1 ; c1ccc(-c2nc3c(o2)-c2cccc4cc(N(c5ccccc5)c5cccc6oc7ccccc7c56)cc-3c24)cc1 ; c1ccc(N(c2cc3c4c(cccc4c2)-c2oc(-c4cc5ccccc5c5ccccc45)nc2-3)c2ccc3oc4ccccc4c3c2)cc1 ; c1ccc(-c2nc3c(o2)-c2cccc4cc(N(c5ccccc5)c5ccc6c(c5)oc5ccccc56)cc-3c24)cc1 ; c1ccc(-c2nc3c(o2)-c2cccc4cc(N(c5ccccc5)c5cccc6c5oc5ccccc56)cc-3c24)cc1]

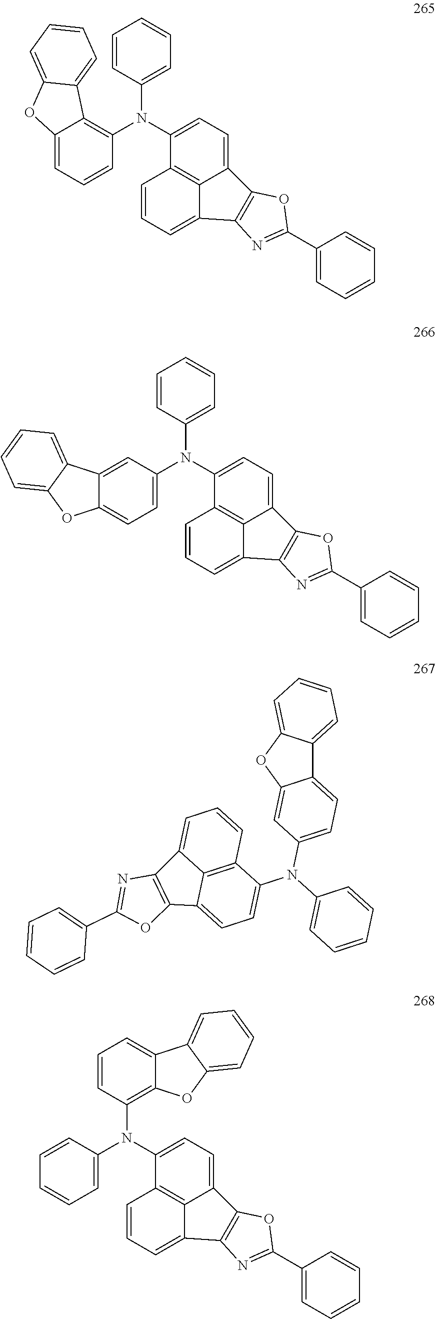 [SMILES: c1ccc(-c2nc3c(o2)-c2ccc(N(c4ccccc4)c4cccc5oc6ccccc6c45)c4cccc-3c24)cc1 ; c1ccc(-c2nc3c(o2)-c2ccc(N(c4ccccc4)c4ccc5oc6ccccc6c5c4)c4cccc-3c24)cc1 ; c1ccc(-c2nc3c(o2)-c2ccc(N(c4ccccc4)c4ccc5c(c4)oc4ccccc45)c4cccc-3c24)cc1 ; c1ccc(-c2nc3c(o2)-c2ccc(N(c4ccccc4)c4cccc5c4oc4ccccc45)c4cccc-3c24)cc1]

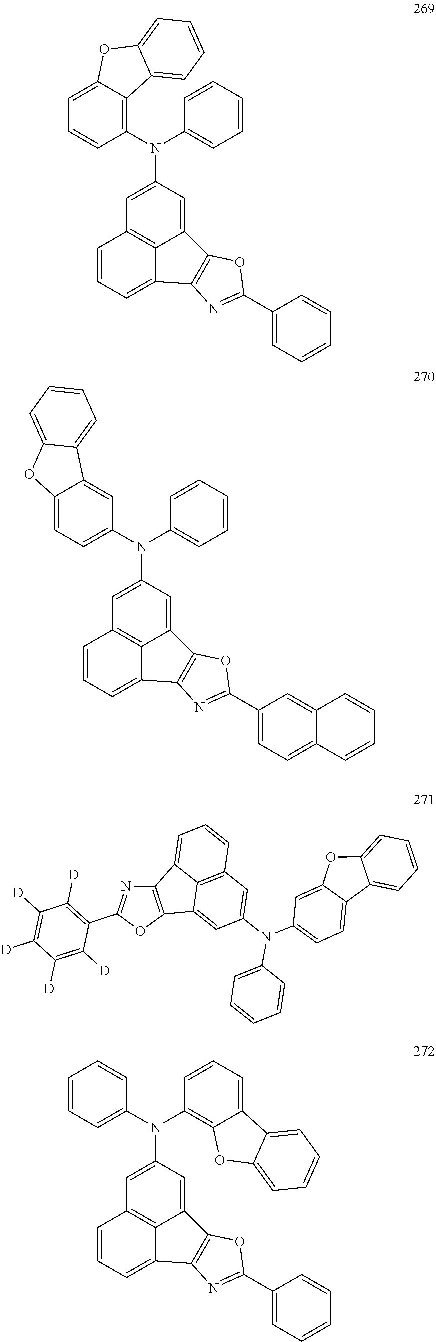 [SMILES: c1ccc(-c2nc3c(o2)-c2cc(N(c4ccccc4)c4cccc5oc6ccccc6c45)cc4cccc-3c24)cc1 ; c1ccc(N(c2cc3c4c(cccc4c2)-c2nc(-c4ccc5ccccc5c4)oc2-3)c2ccc3oc4ccccc4c3c2)cc1 ; [2H]c1c([2H])c([2H])c(-c2nc3c(o2)-c2cc(N(c4ccccc4)c4ccc5c(c4)oc4ccccc45)cc4cccc-3c24)c([2H])c1[2H] ; c1ccc(-c2nc3c(o2)-c2cc(N(c4ccccc4)c4cccc5c4oc4ccccc45)cc4cccc-3c24)cc1]

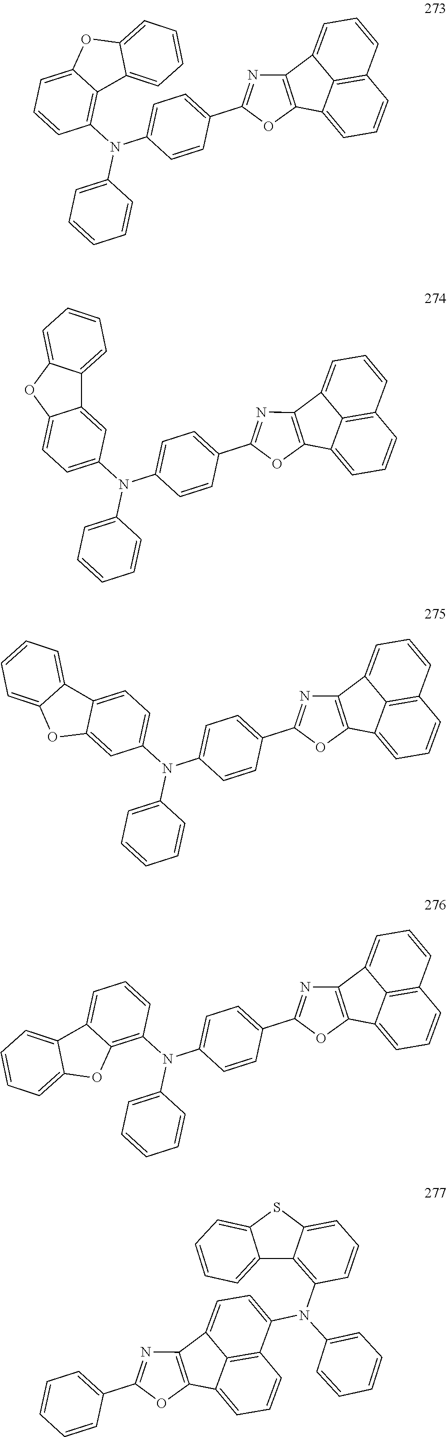 [SMILES: c1ccc(N(c2ccc(-c3nc4c(o3)-c3cccc5cccc-4c35)cc2)c2cccc3oc4ccccc4c23)cc1 ; c1ccc(N(c2ccc(-c3nc4c(o3)-c3cccc5cccc-4c35)cc2)c2ccc3oc4ccccc4c3c2)cc1 ; c1ccc(N(c2ccc(-c3nc4c(o3)-c3cccc5cccc-4c35)cc2)c2ccc3c(c2)oc2ccccc23)cc1 ; c1ccc(N(c2ccc(-c3nc4c(o3)-c3cccc5cccc-4c35)cc2)c2cccc3c2oc2ccccc23)cc1 ; c1ccc(-c2nc3c(o2)-c2cccc4c(N(c5ccccc5)c5cccc6sc7ccccc7c56)ccc-3c24)cc1]

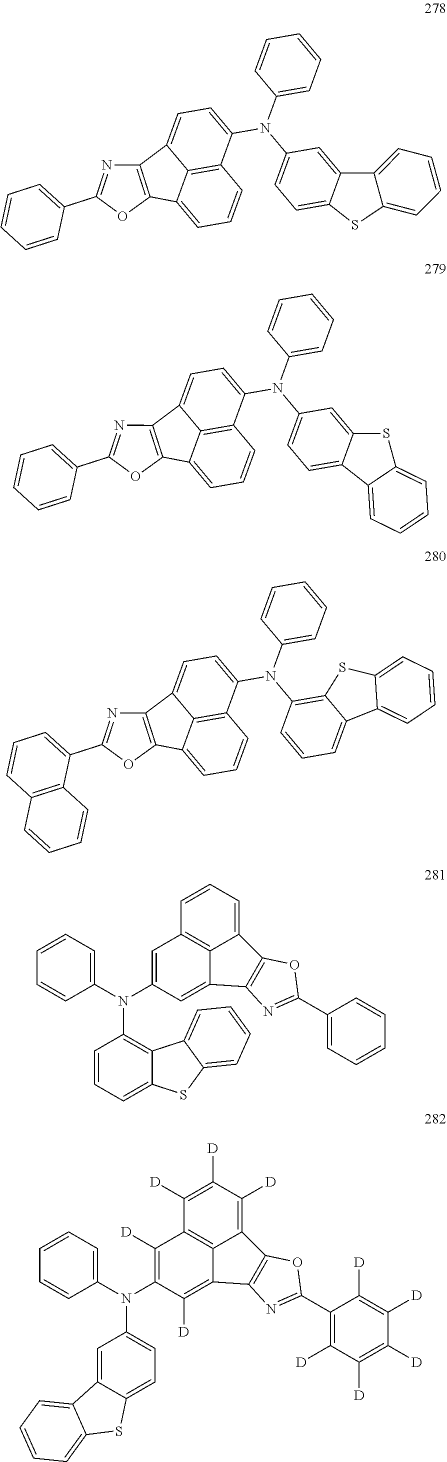

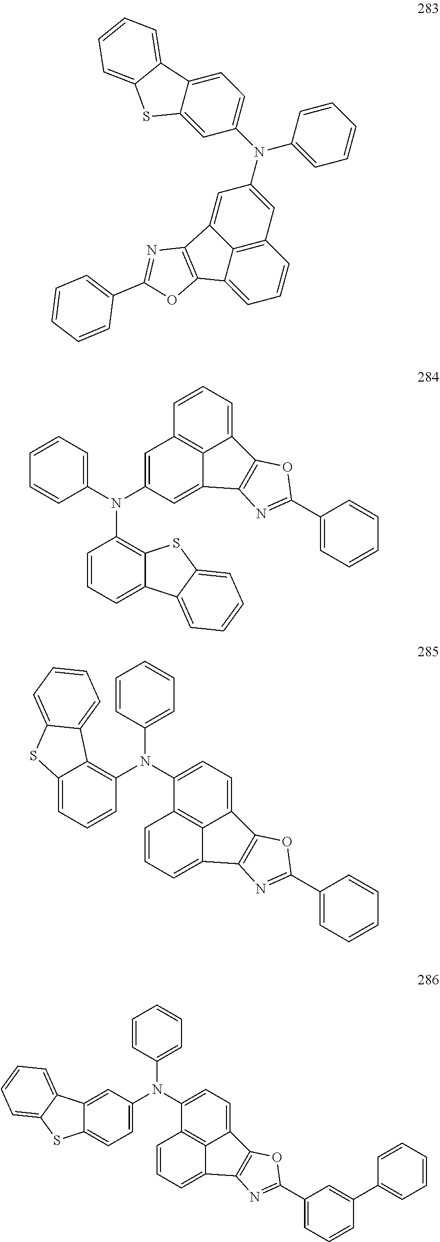 [SMILES: c1ccc(-c2nc3c(o2)-c2cccc4cc(N(c5ccccc5)c5ccc6c(c5)sc5ccccc56)cc-3c24)cc1 ; c1ccc(-c2nc3c(o2)-c2cccc4cc(N(c5ccccc5)c5cccc6c5sc5ccccc56)cc-3c24)cc1 ; c1ccc(-c2nc3c(o2)-c2ccc(N(c4ccccc4)c4cccc5sc6ccccc6c45)c4cccc-3c24)cc1 ; c1ccc(-c2cccc(-c3nc4c(o3)-c3ccc(N(c5ccccc5)c5ccc6sc7ccccc7c6c5)c5cccc-4c35)c2)cc1]

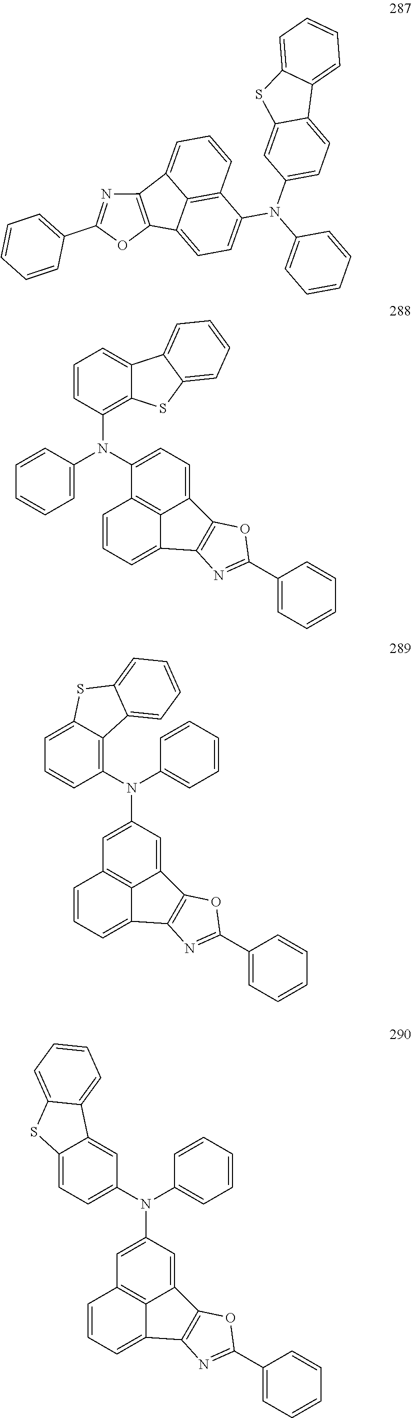 [SMILES: c1ccc(-c2nc3c(o2)-c2ccc(N(c4ccccc4)c4ccc5c(c4)sc4ccccc45)c4cccc-3c24)cc1 ; c1ccc(-c2nc3c(o2)-c2ccc(N(c4ccccc4)c4cccc5c4sc4ccccc45)c4cccc-3c24)cc1 ; c1ccc(-c2nc3c(o2)-c2cc(N(c4ccccc4)c4cccc5sc6ccccc6c45)cc4cccc-3c24)cc1 ; c1ccc(-c2nc3c(o2)-c2cc(N(c4ccccc4)c4ccc5sc6ccccc6c5c4)cc4cccc-3c24)cc1]

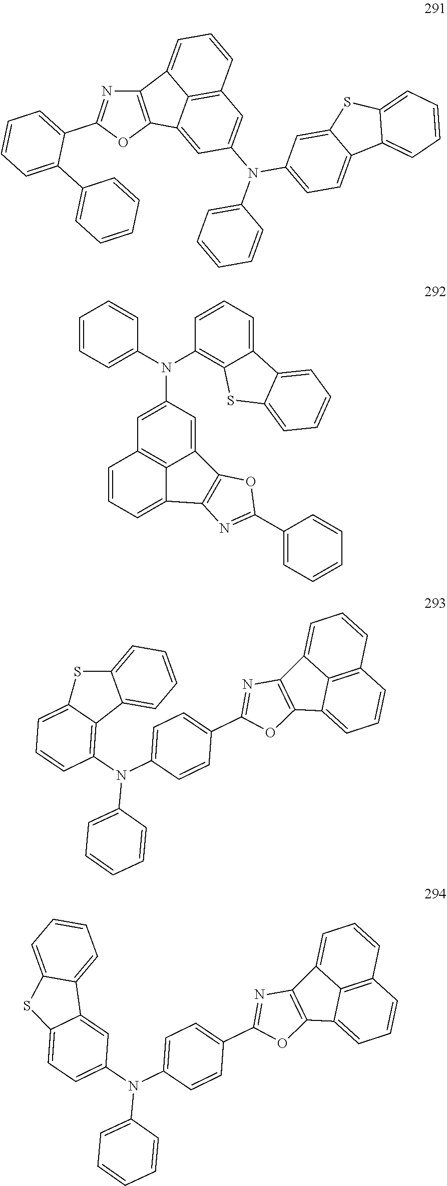

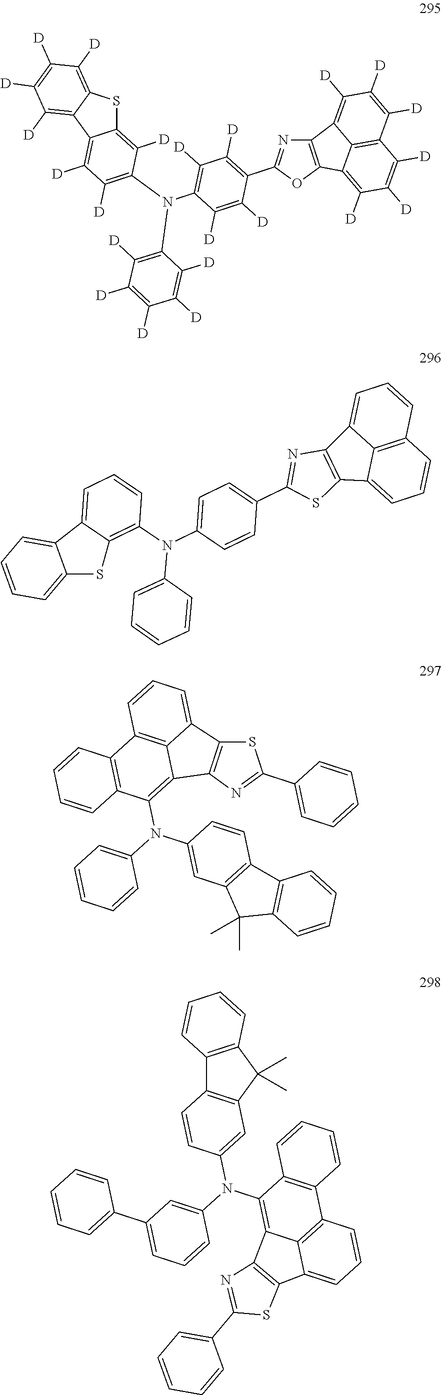

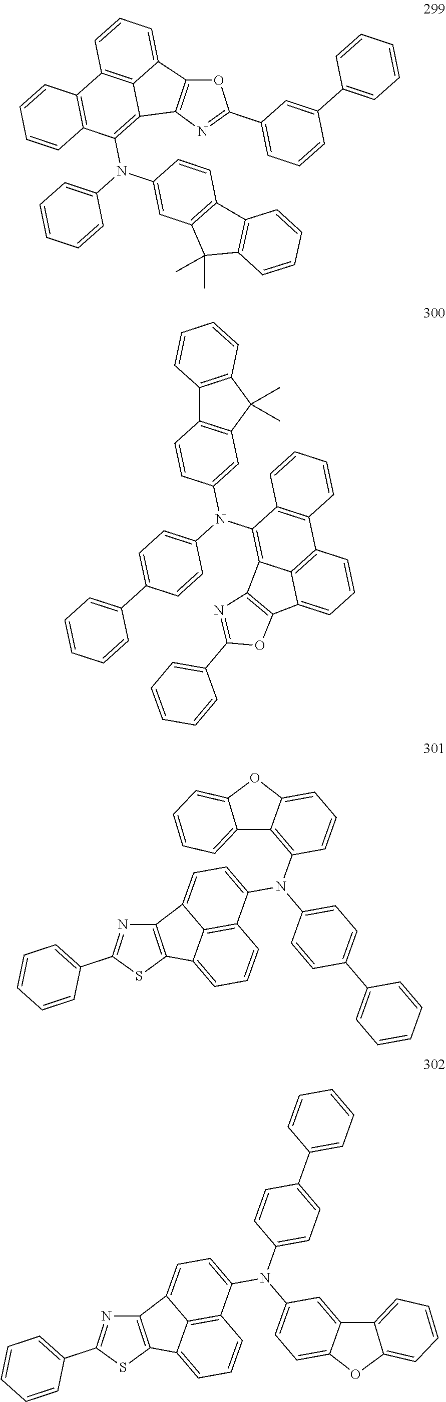

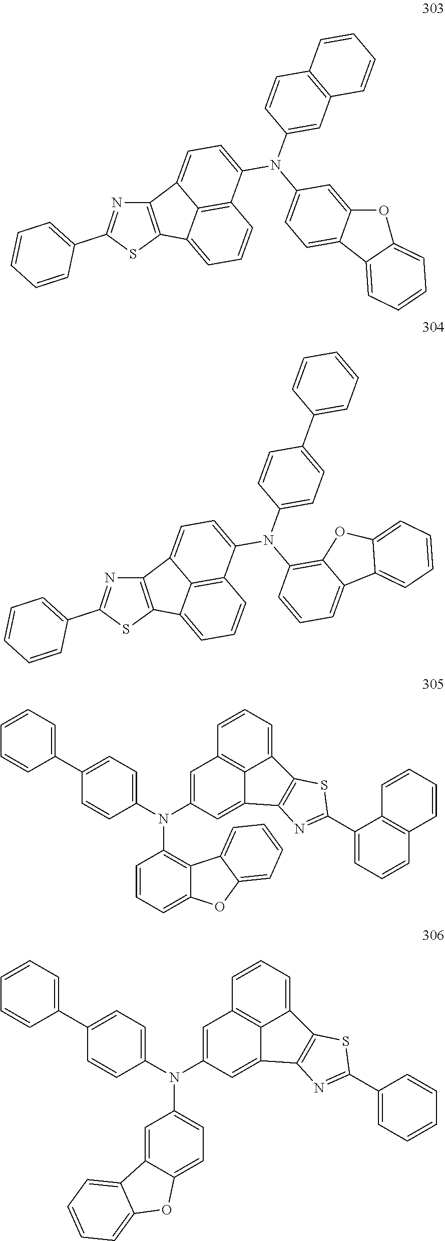 [SMILES: c1ccc(-c2nc3c(s2)-c2cccc4c(N(c5ccc6ccccc6c5)c5ccc6c(c5)oc5ccccc56)ccc-3c24)cc1 ; c1ccc(-c2ccc(N(c3ccc4c5c(cccc35)-c3sc(-c5ccccc5)nc3-4)c3cccc4c3oc3ccccc34)cc2)cc1 ; c1ccc(-c2ccc(N(c3cc4c5c(cccc5c3)-c3sc(-c5cccc6ccccc56)nc3-4)c3cccc4oc5ccccc5c34)cc2)cc1 ; c1ccc(-c2ccc(N(c3cc4c5c(cccc5c3)-c3sc(-c5ccccc5)nc3-4)c3ccc4oc5ccccc5c4c3)cc2)cc1]

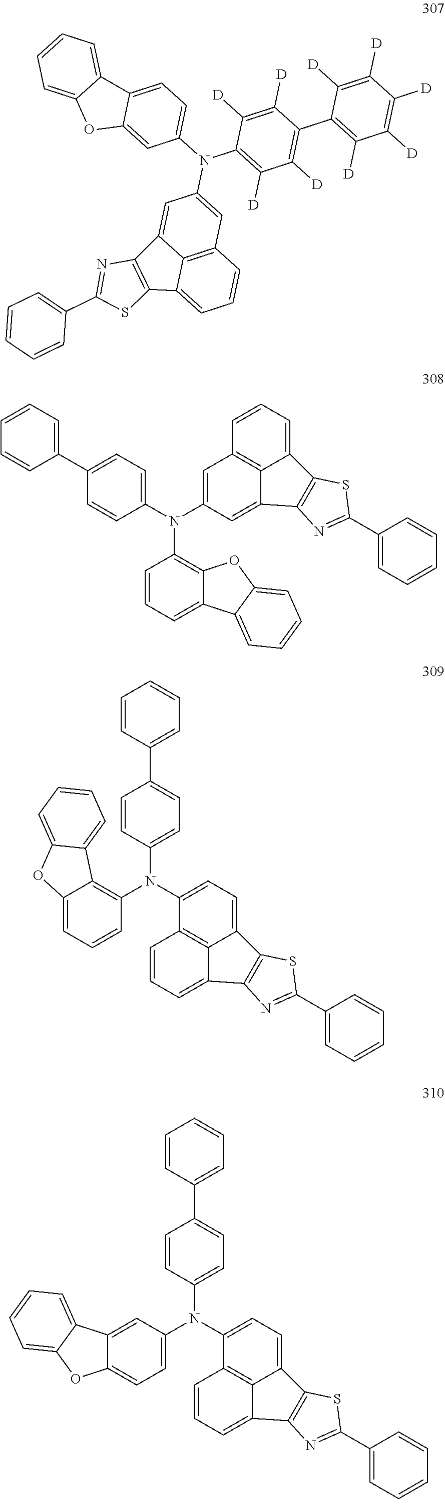

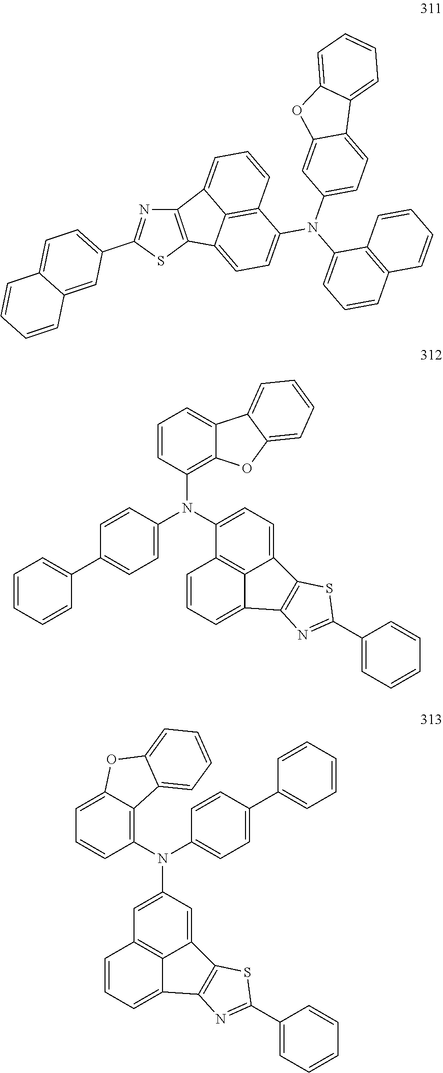 [SMILES: c1ccc2cc(-c3nc4c(s3)-c3ccc(N(c5ccc6c(c5)oc5ccccc56)c5cccc6ccccc56)c5cccc-4c35)ccc2c1 ; c1ccc(-c2ccc(N(c3ccc4c5c(cccc35)-c3nc(-c5ccccc5)sc3-4)c3cccc4c3oc3ccccc34)cc2)cc1 ; c1ccc(-c2ccc(N(c3cc4c5c(cccc5c3)-c3nc(-c5ccccc5)sc3-4)c3cccc4oc5ccccc5c34)cc2)cc1]

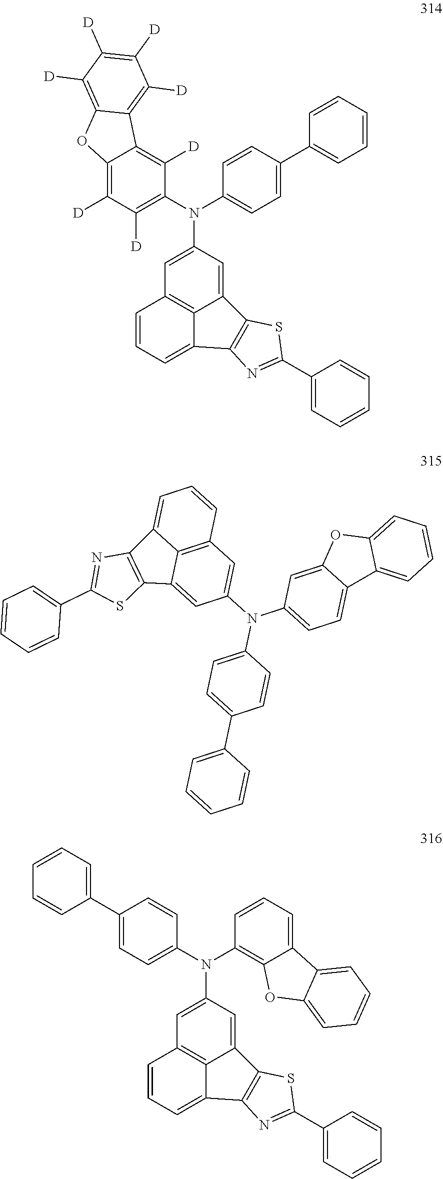 [SMILES: [2H]c1c([2H])c([2H])c2c(oc3c([2H])c([2H])c(N(c4ccc(-c5ccccc5)cc4)c4cc5c6c(cccc6c4)-c4nc(-c6ccccc6)sc4-5)c([2H])c32)c1[2H] ; c1ccc(-c2ccc(N(c3cc4c5c(cccc5c3)-c3nc(-c5ccccc5)sc3-4)c3ccc4c(c3)oc3ccccc34)cc2)cc1 ; c1ccc(-c2ccc(N(c3cc4c5c(cccc5c3)-c3nc(-c5ccccc5)sc3-4)c3cccc4c3oc3ccccc34)cc2)cc1]

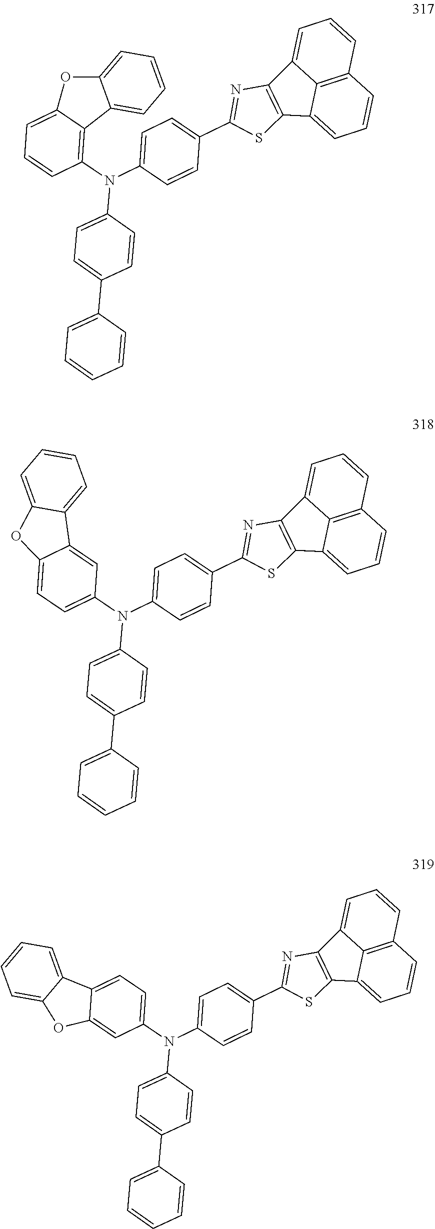 [SMILES: c1ccc(-c2ccc(N(c3ccc(-c4nc5c(s4)-c4cccc6cccc-5c46)cc3)c3cccc4oc5ccccc5c34)cc2)cc1 ; c1ccc(-c2ccc(N(c3ccc(-c4nc5c(s4)-c4cccc6cccc-5c46)cc3)c3ccc4oc5ccccc5c4c3)cc2)cc1 ; c1ccc(-c2ccc(N(c3ccc(-c4nc5c(s4)-c4cccc6cccc-5c46)cc3)c3ccc4c(c3)oc3ccccc34)cc2)cc1]

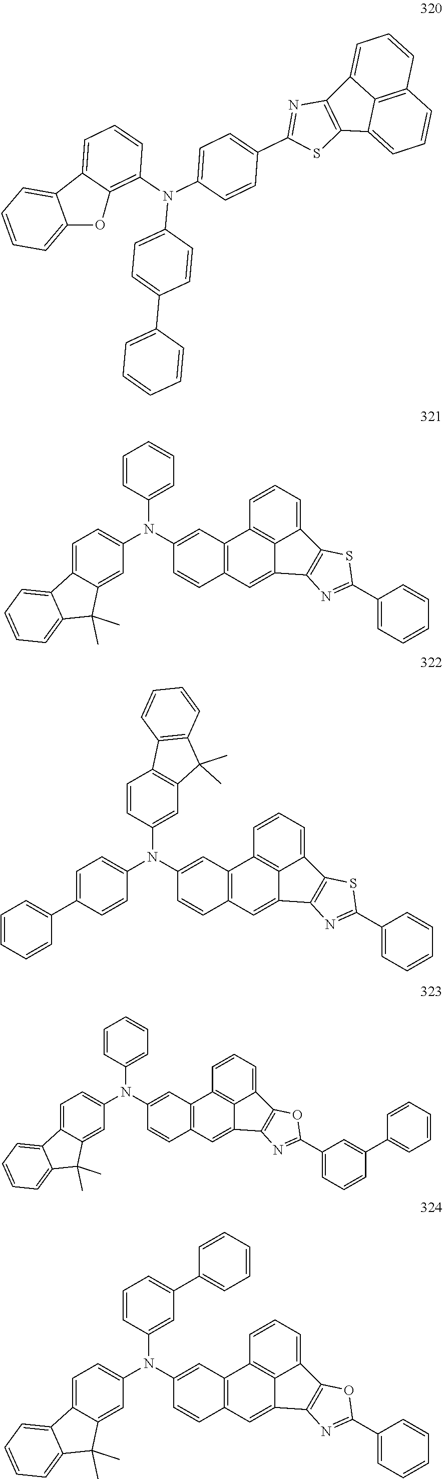 [SMILES: c1ccc(-c2ccc(N(c3ccc(-c4nc5c(s4)-c4cccc6cccc-5c46)cc3)c3cccc4c3oc3ccccc34)cc2)cc1 ; CC1(C)c2ccccc2-c2ccc(N(c3ccccc3)c3ccc4cc5c6c(cccc6c4c3)-c3sc(-c4ccccc4)nc3-5)cc21 ; CC1(C)c2ccccc2-c2ccc(N(c3ccc(-c4ccccc4)cc3)c3ccc4cc5c6c(cccc6c4c3)-c3sc(-c4ccccc4)nc3-5)cc21 ; CC1(C)c2ccccc2-c2ccc(N(c3ccccc3)c3ccc4cc5c6c(cccc6c4c3)-c3oc(-c4cccc(-c6ccccc6)c4)nc3-5)cc21 ; CC1(C)c2ccccc2-c2ccc(N(c3cccc(-c4ccccc4)c3)c3ccc4cc5c6c(cccc6c4c3)-c3oc(-c4ccccc4)nc3-5)cc21]

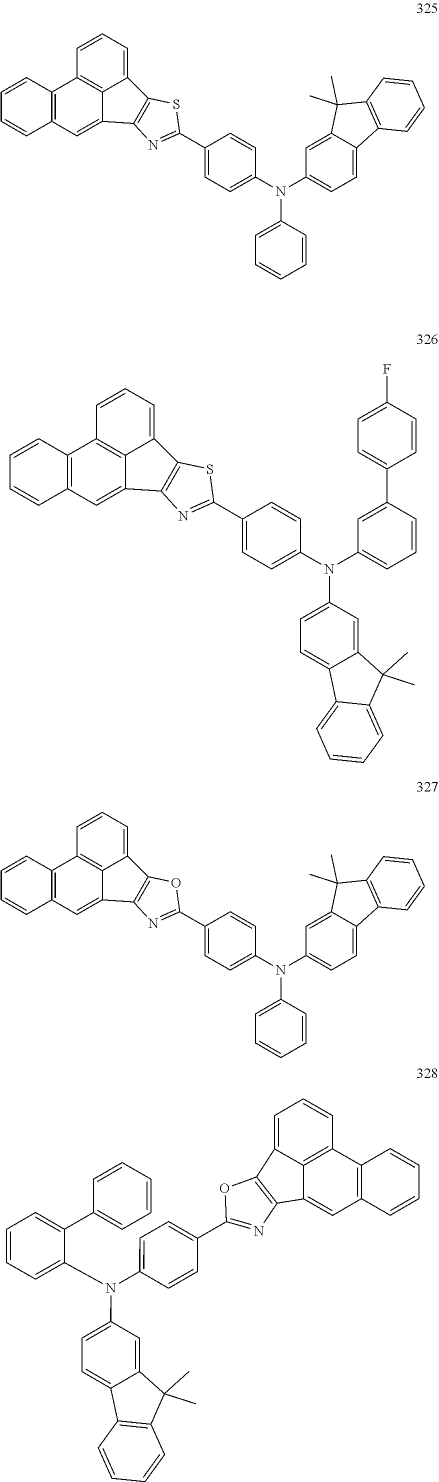 [SMILES: CC1(C)c2ccccc2-c2ccc(N(c3ccccc3)c3ccc(-c4nc5c(s4)-c4cccc6c4c-5cc4ccccc46)cc3)cc21 ; CC1(C)c2ccccc2-c2ccc(N(c3ccc(-c4nc5c(s4)-c4cccc6c4c-5cc4ccccc46)cc3)c3cccc(-c4ccc(F)cc4)c3)cc21 ; CC1(C)c2ccccc2-c2ccc(N(c3ccccc3)c3ccc(-c4nc5c(o4)-c4cccc6c4c-5cc4ccccc46)cc3)cc21 ; CC1(C)c2ccccc2-c2ccc(N(c3ccc(-c4nc5c(o4)-c4cccc6c4c-5cc4ccccc46)cc3)c3ccccc3-c3ccccc3)cc21]

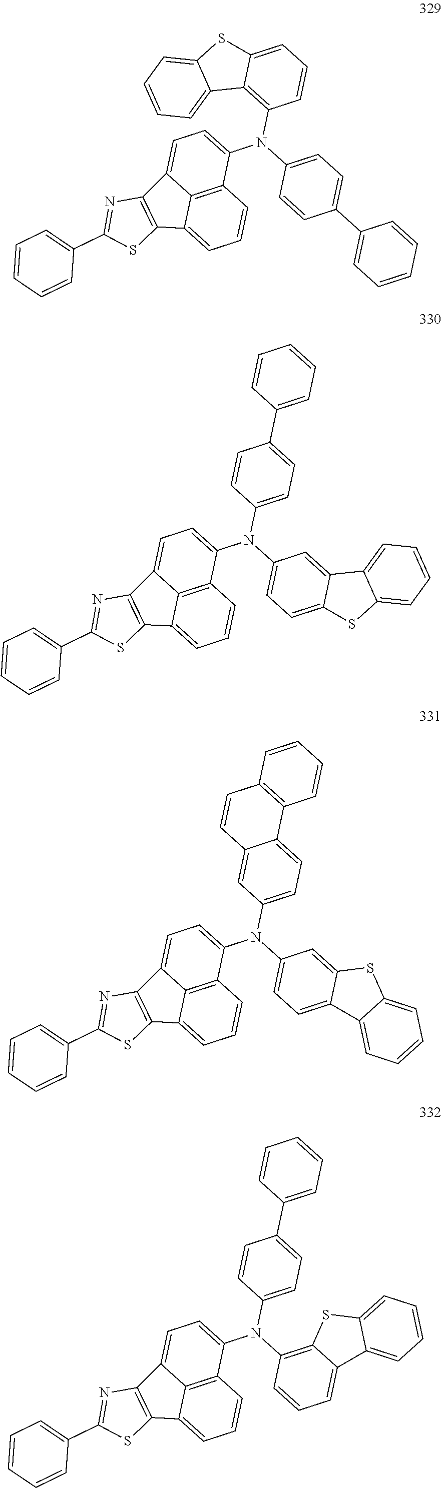 [SMILES: c1ccc(-c2ccc(N(c3ccc4c5c(cccc35)-c3sc(-c5ccccc5)nc3-4)c3cccc4sc5ccccc5c34)cc2)cc1 ; c1ccc(-c2ccc(N(c3ccc4sc5ccccc5c4c3)c3ccc4c5c(cccc35)-c3sc(-c5ccccc5)nc3-4)cc2)cc1 ; c1ccc(-c2nc3c(s2)-c2cccc4c(N(c5ccc6c(ccc7ccccc76)c5)c5ccc6c(c5)sc5ccccc56)ccc-3c24)cc1 ; c1ccc(-c2ccc(N(c3ccc4c5c(cccc35)-c3sc(-c5ccccc5)nc3-4)c3cccc4c3sc3ccccc34)cc2)cc1]

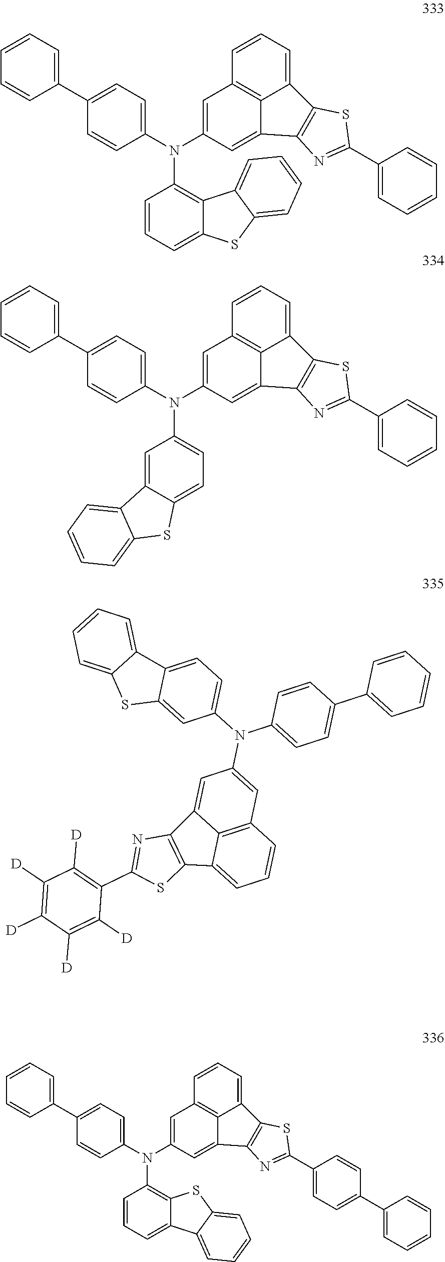

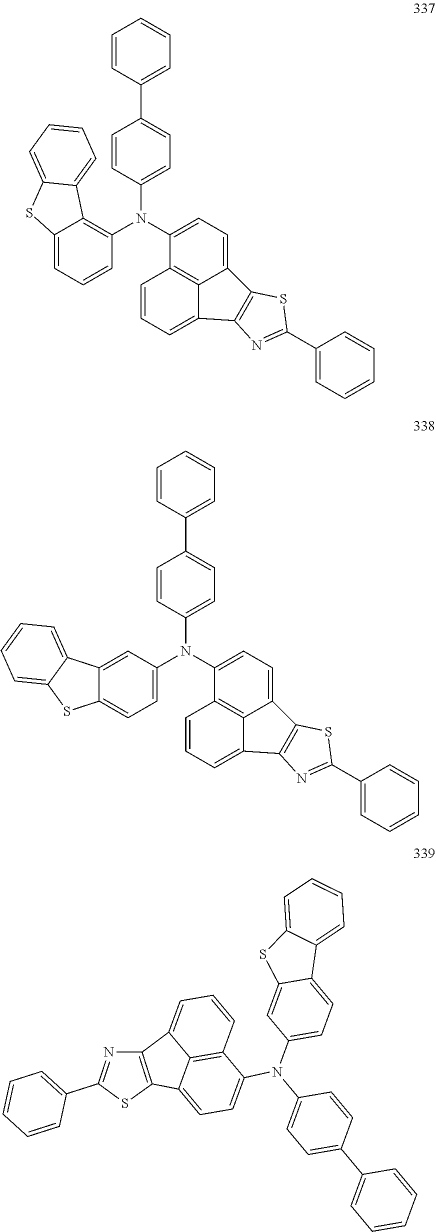 [SMILES: c1ccc(-c2ccc(N(c3ccc4c5c(cccc35)-c3nc(-c5ccccc5)sc3-4)c3cccc4sc5ccccc5c34)cc2)cc1 ; c1ccc(-c2ccc(N(c3ccc4sc5ccccc5c4c3)c3ccc4c5c(cccc35)-c3nc(-c5ccccc5)sc3-4)cc2)cc1 ; c1ccc(-c2ccc(N(c3ccc4c(c3)sc3ccccc34)c3ccc4c5c(cccc35)-c3nc(-c5ccccc5)sc3-4)cc2)cc1]

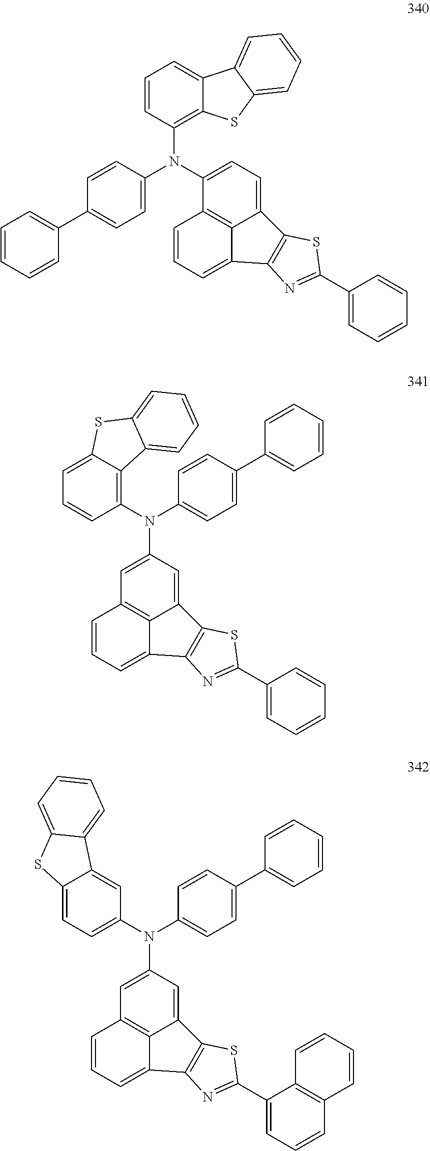 [SMILES: c1ccc(-c2ccc(N(c3ccc4c5c(cccc35)-c3nc(-c5ccccc5)sc3-4)c3cccc4c3sc3ccccc34)cc2)cc1 ; c1ccc(-c2ccc(N(c3cc4c5c(cccc5c3)-c3nc(-c5ccccc5)sc3-4)c3cccc4sc5ccccc5c34)cc2)cc1 ; c1ccc(-c2ccc(N(c3cc4c5c(cccc5c3)-c3nc(-c5cccc6ccccc56)sc3-4)c3ccc4sc5ccccc5c4c3)cc2)cc1]

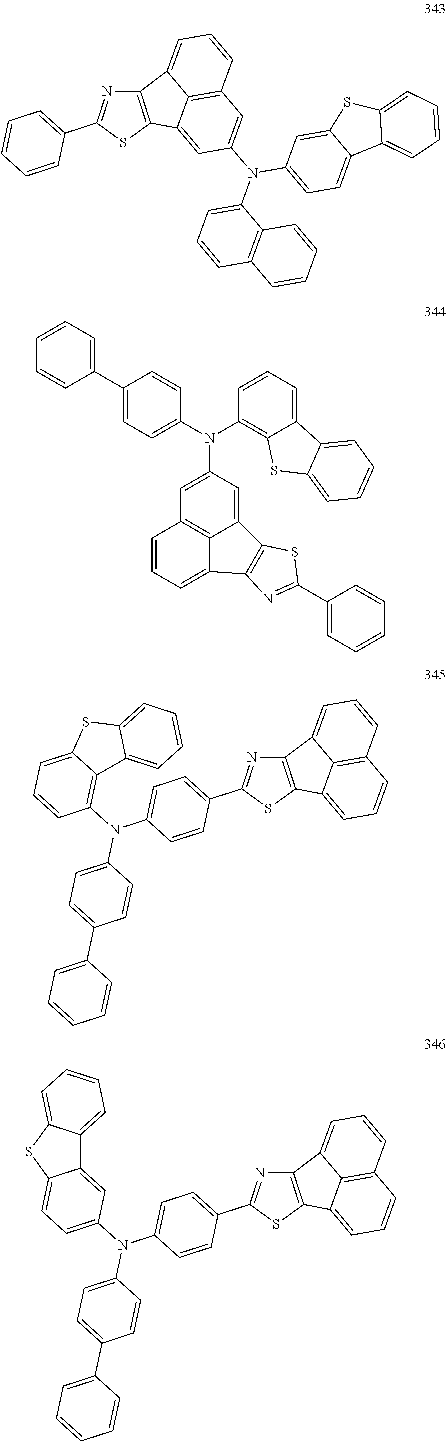

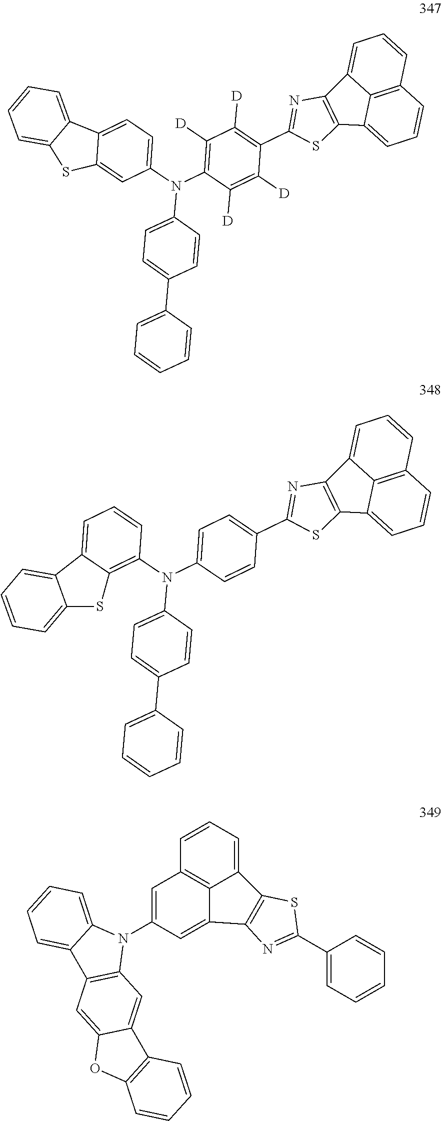 [SMILES: [2H]c1c([2H])c(N(c2ccc(-c3ccccc3)cc2)c2ccc3c(c2)sc2ccccc23)c([2H])c([2H])c1-c1nc2c(s1)-c1cccc3cccc-2c13 ; c1ccc(-c2ccc(N(c3ccc(-c4nc5c(s4)-c4cccc6cccc-5c46)cc3)c3cccc4c3sc3ccccc34)cc2)cc1 ; c1ccc(-c2nc3c(s2)-c2cccc4cc(-n5c6ccccc6c6cc7oc8ccccc8c7cc65)cc-3c24)cc1]

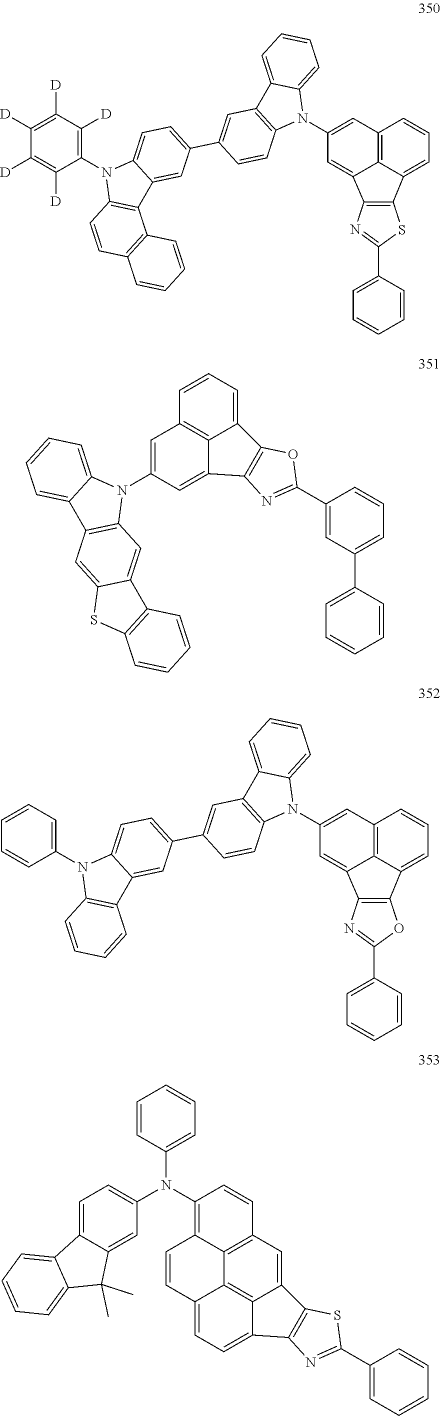 [SMILES: [2H]c1c([2H])c([2H])c(-n2c3ccc(-c4ccc5c(c4)c4ccccc4n5-c4cc5c6c(cccc6c4)-c4sc(-c6ccccc6)nc4-5)cc3c3c4ccccc4ccc32)c([2H])c1[2H] ; c1ccc(-c2cccc(-c3nc4c(o3)-c3cccc5cc(-n6c7ccccc7c7cc8sc9ccccc9c8cc76)cc-4c35)c2)cc1 ; c1ccc(-c2nc3c(o2)-c2cccc4cc(-n5c6ccccc6c6cc(-c7ccc8c(c7)c7ccccc7n8-c7ccccc7)ccc65)cc-3c24)cc1 ; CC1(C)c2ccccc2-c2ccc(N(c3ccccc3)c3ccc4cc5c6c(ccc7ccc3c4c76)-c3nc(-c4ccccc4)sc3-5)cc21]

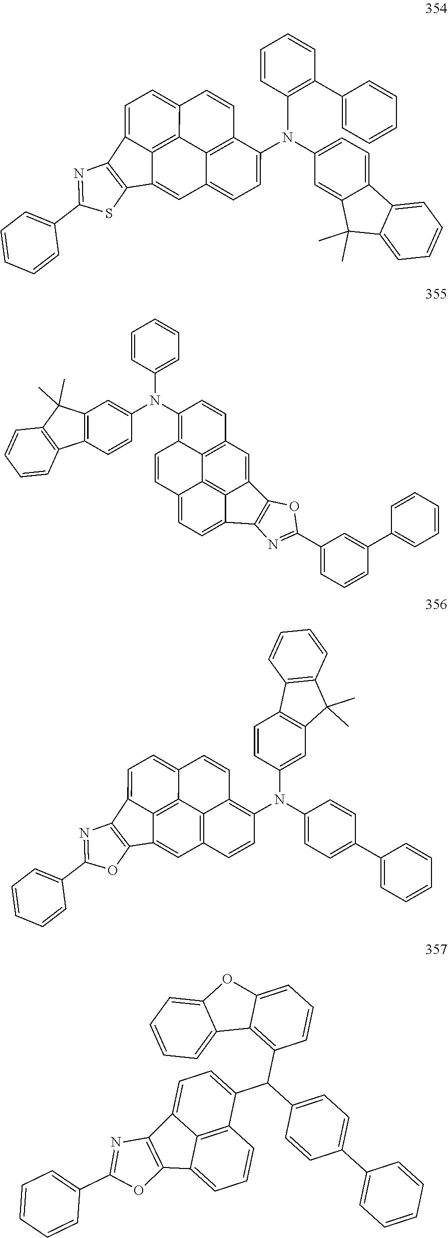 [SMILES: CC1(C)c2ccccc2-c2ccc(N(c3ccccc3-c3ccccc3)c3ccc4cc5c6c(ccc7ccc3c4c76)-c3nc(-c4ccccc4)sc3-5)cc21 ; CC1(C)c2ccccc2-c2ccc(N(c3ccccc3)c3ccc4cc5c6c(ccc7ccc3c4c76)-c3nc(-c4cccc(-c6ccccc6)c4)oc3-5)cc21 ; CC1(C)c2ccccc2-c2ccc(N(c3ccc(-c4ccccc4)cc3)c3ccc4cc5c6c(ccc7ccc3c4c76)-c3nc(-c4ccccc4)oc3-5)cc21 ; c1ccc(-c2ccc(C(c3ccc4c5c(cccc35)-c3oc(-c5ccccc5)nc3-4)c3cccc4oc5ccccc5c34)cc2)cc1]

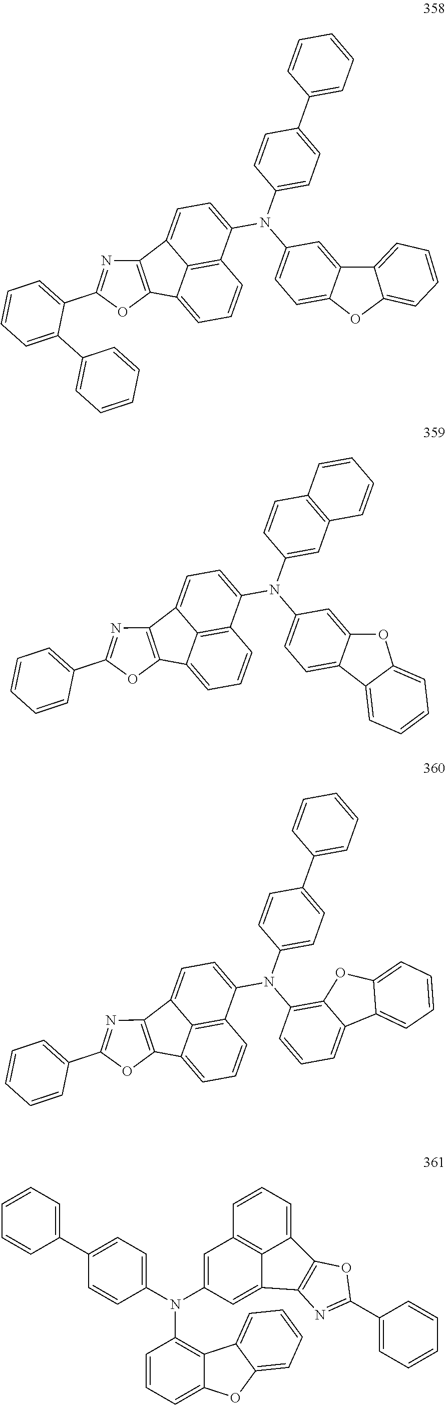 [SMILES: c1ccc(-c2ccc(N(c3ccc4oc5ccccc5c4c3)c3ccc4c5c(cccc35)-c3oc(-c5ccccc5-c5ccccc5)nc3-4)cc2)cc1 ; c1ccc(-c2nc3c(o2)-c2cccc4c(N(c5ccc6ccccc6c5)c5ccc6c(c5)oc5ccccc56)ccc-3c24)cc1 ; c1ccc(-c2ccc(N(c3ccc4c5c(cccc35)-c3oc(-c5ccccc5)nc3-4)c3cccc4c3oc3ccccc34)cc2)cc1 ; c1ccc(-c2ccc(N(c3cc4c5c(cccc5c3)-c3oc(-c5ccccc5)nc3-4)c3cccc4oc5ccccc5c34)cc2)cc1]

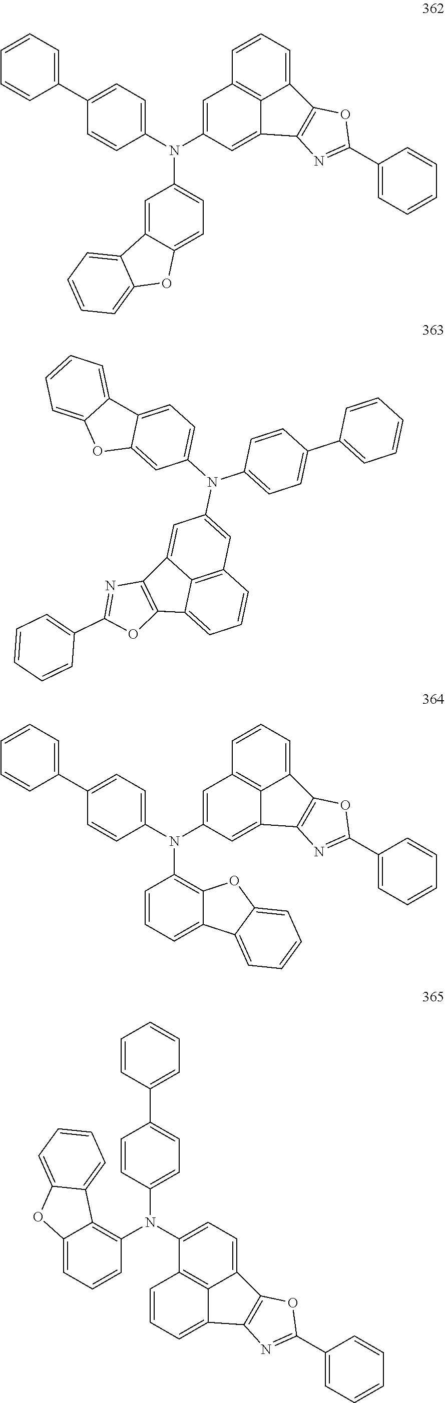

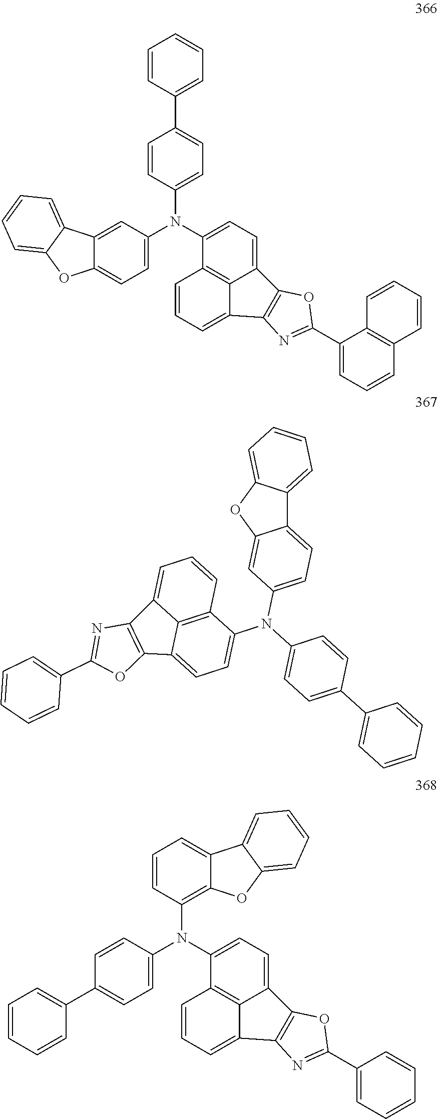 [SMILES: c1ccc(-c2ccc(N(c3ccc4oc5ccccc5c4c3)c3ccc4c5c(cccc35)-c3nc(-c5cccc6ccccc56)oc3-4)cc2)cc1 ; c1ccc(-c2ccc(N(c3ccc4c(c3)oc3ccccc34)c3ccc4c5c(cccc35)-c3nc(-c5ccccc5)oc3-4)cc2)cc1 ; c1ccc(-c2ccc(N(c3ccc4c5c(cccc35)-c3nc(-c5ccccc5)oc3-4)c3cccc4c3oc3ccccc34)cc2)cc1]

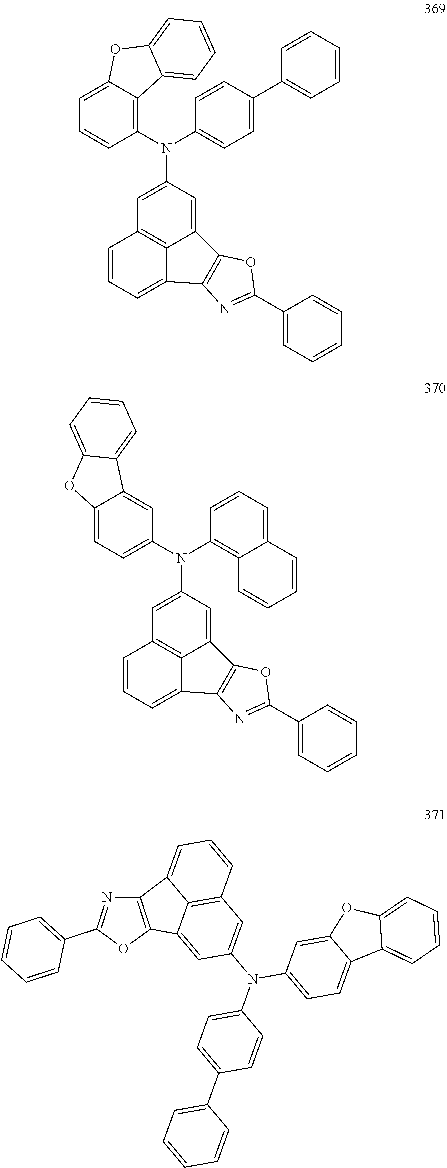 [SMILES: c1ccc(-c2ccc(N(c3cc4c5c(cccc5c3)-c3nc(-c5ccccc5)oc3-4)c3cccc4oc5ccccc5c34)cc2)cc1 ; c1ccc(-c2nc3c(o2)-c2cc(N(c4ccc5oc6ccccc6c5c4)c4cccc5ccccc45)cc4cccc-3c24)cc1 ; c1ccc(-c2ccc(N(c3cc4c5c(cccc5c3)-c3nc(-c5ccccc5)oc3-4)c3ccc4c(c3)oc3ccccc34)cc2)cc1]

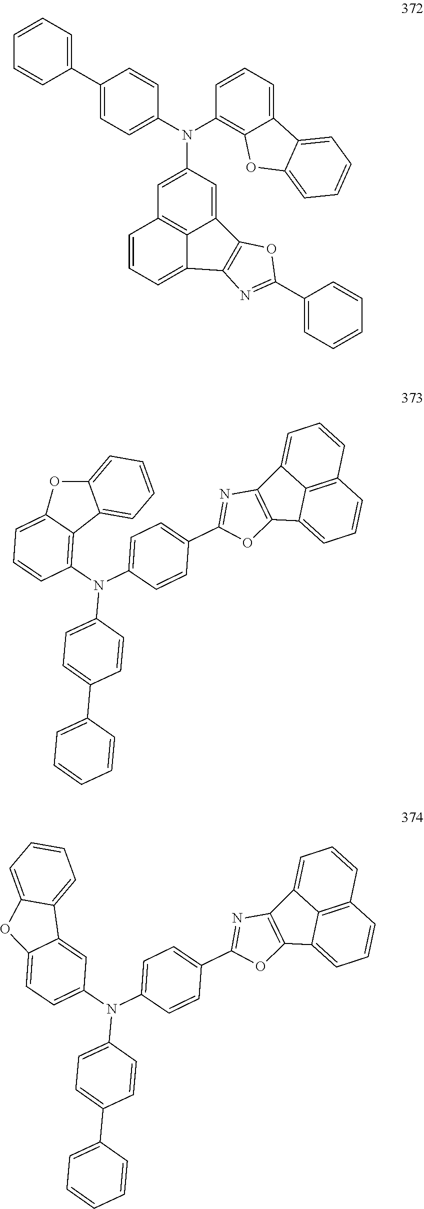 [SMILES: c1ccc(-c2ccc(N(c3cc4c5c(cccc5c3)-c3nc(-c5ccccc5)oc3-4)c3cccc4c3oc3ccccc34)cc2)cc1 ; c1ccc(-c2ccc(N(c3ccc(-c4nc5c(o4)-c4cccc6cccc-5c46)cc3)c3cccc4oc5ccccc5c34)cc2)cc1 ; c1ccc(-c2ccc(N(c3ccc(-c4nc5c(o4)-c4cccc6cccc-5c46)cc3)c3ccc4oc5ccccc5c4c3)cc2)cc1]

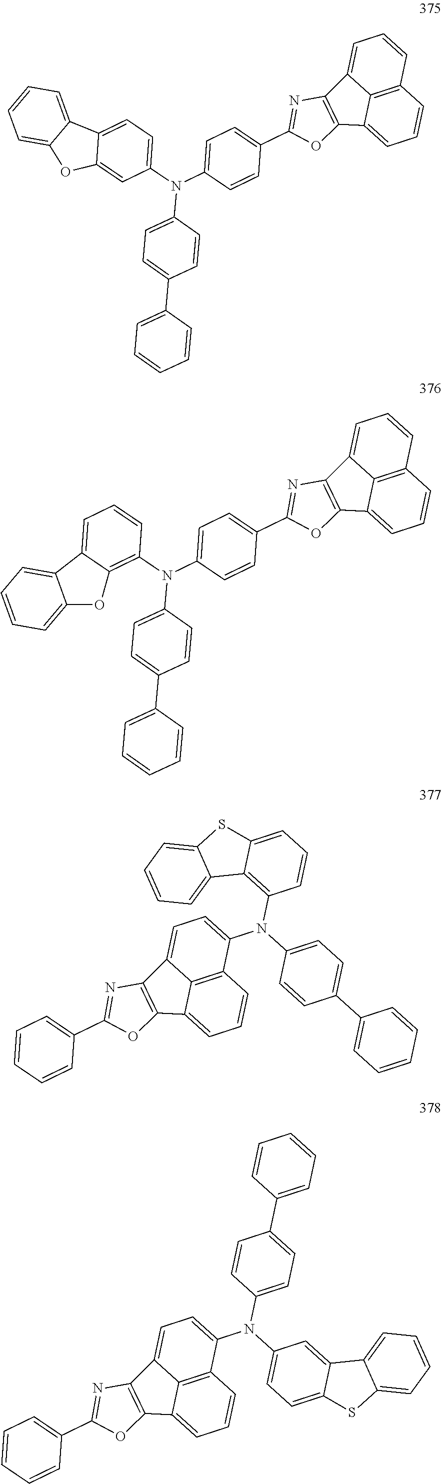 [SMILES: c1ccc(-c2ccc(N(c3ccc(-c4nc5c(o4)-c4cccc6cccc-5c46)cc3)c3ccc4c(c3)oc3ccccc34)cc2)cc1 ; c1ccc(-c2ccc(N(c3ccc(-c4nc5c(o4)-c4cccc6cccc-5c46)cc3)c3cccc4c3oc3ccccc34)cc2)cc1 ; c1ccc(-c2ccc(N(c3ccc4c5c(cccc35)-c3oc(-c5ccccc5)nc3-4)c3cccc4sc5ccccc5c34)cc2)cc1 ; c1ccc(-c2ccc(N(c3ccc4sc5ccccc5c4c3)c3ccc4c5c(cccc35)-c3oc(-c5ccccc5)nc3-4)cc2)cc1]

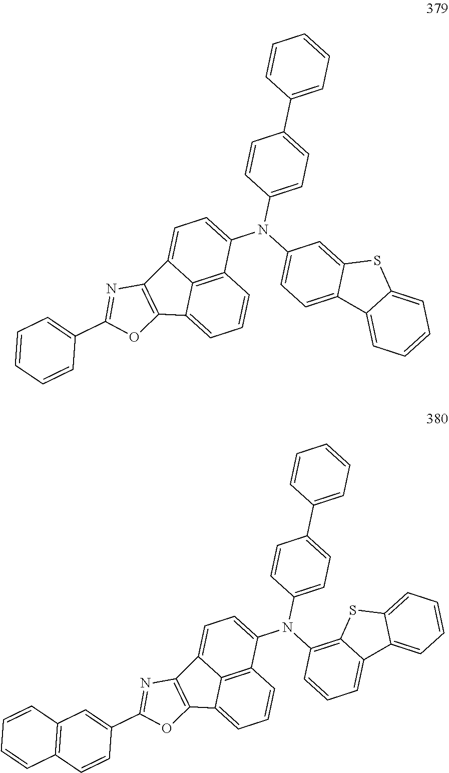 [SMILES: c1ccc(-c2ccc(N(c3ccc4c(c3)sc3ccccc34)c3ccc4c5c(cccc35)-c3oc(-c5ccccc5)nc3-4)cc2)cc1 ; c1ccc(-c2ccc(N(c3ccc4c5c(cccc35)-c3oc(-c5ccc6ccccc6c5)nc3-4)c3cccc4c3sc3ccccc34)cc2)cc1]

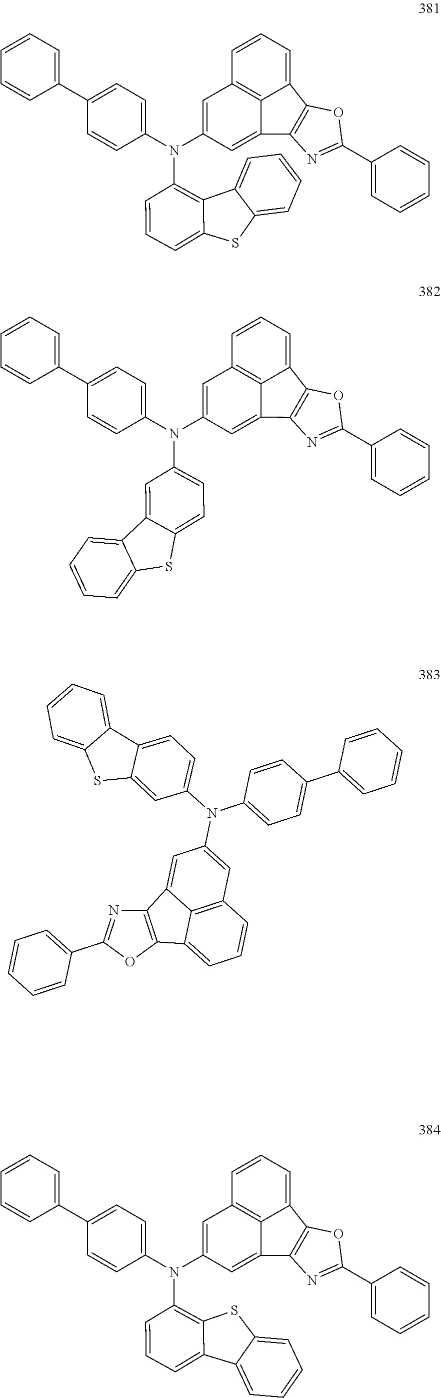 [SMILES: c1ccc(-c2ccc(N(c3cc4c5c(cccc5c3)-c3oc(-c5ccccc5)nc3-4)c3cccc4sc5ccccc5c34)cc2)cc1 ; c1ccc(-c2ccc(N(c3cc4c5c(cccc5c3)-c3oc(-c5ccccc5)nc3-4)c3ccc4sc5ccccc5c4c3)cc2)cc1 ; c1ccc(-c2ccc(N(c3cc4c5c(cccc5c3)-c3oc(-c5ccccc5)nc3-4)c3ccc4c(c3)sc3ccccc34)cc2)cc1 ; c1ccc(-c2ccc(N(c3cc4c5c(cccc5c3)-c3oc(-c5ccccc5)nc3-4)c3cccc4c3sc3ccccc34)cc2)cc1]

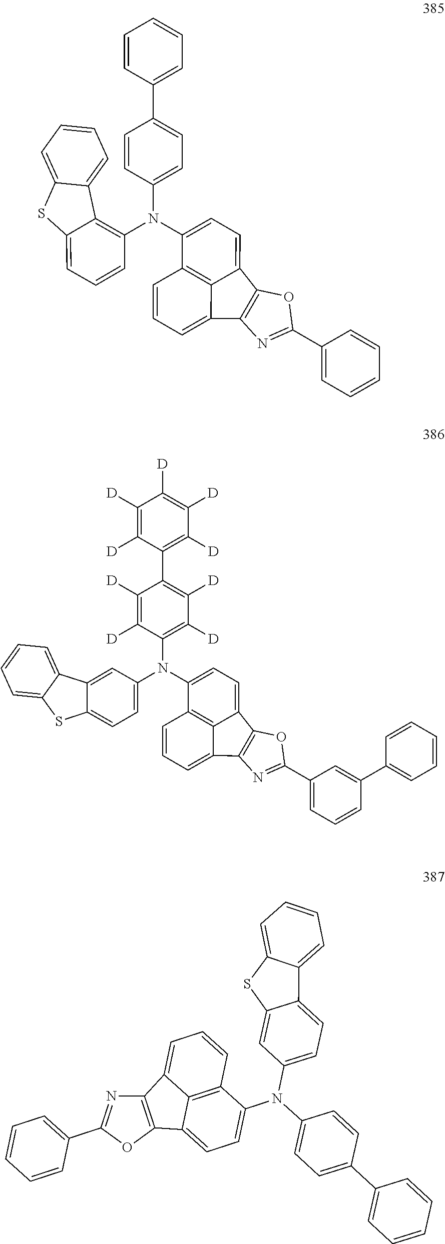 [SMILES: c1ccc(-c2ccc(N(c3ccc4c5c(cccc35)-c3nc(-c5ccccc5)oc3-4)c3cccc4sc5ccccc5c34)cc2)cc1 ; [2H]c1c([2H])c([2H])c(-c2c([2H])c([2H])c(N(c3ccc4sc5ccccc5c4c3)c3ccc4c5c(cccc35)-c3nc(-c5cccc(-c6ccccc6)c5)oc3-4)c([2H])c2[2H])c([2H])c1[2H] ; c1ccc(-c2ccc(N(c3ccc4c(c3)sc3ccccc34)c3ccc4c5c(cccc35)-c3nc(-c5ccccc5)oc3-4)cc2)cc1]

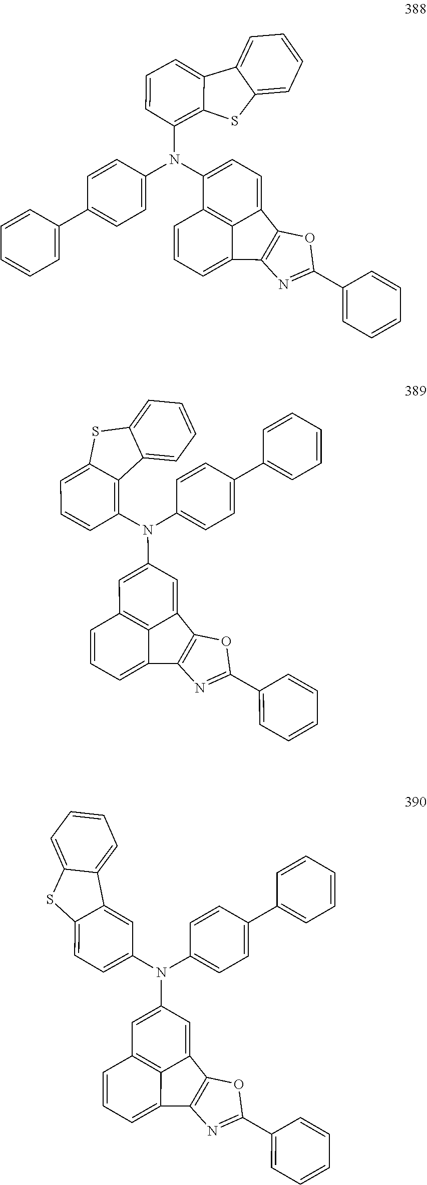 [SMILES: c1ccc(-c2ccc(N(c3ccc4c5c(cccc35)-c3nc(-c5ccccc5)oc3-4)c3cccc4c3sc3ccccc34)cc2)cc1 ; c1ccc(-c2ccc(N(c3cc4c5c(cccc5c3)-c3nc(-c5ccccc5)oc3-4)c3cccc4sc5ccccc5c34)cc2)cc1 ; c1ccc(-c2ccc(N(c3cc4c5c(cccc5c3)-c3nc(-c5ccccc5)oc3-4)c3ccc4sc5ccccc5c4c3)cc2)cc1]

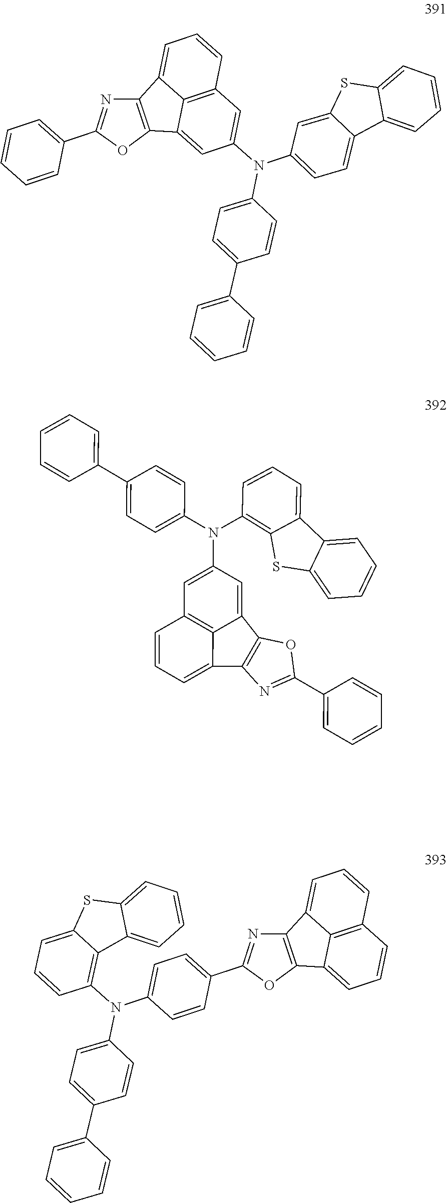 [SMILES: c1ccc(-c2ccc(N(c3cc4c5c(cccc5c3)-c3nc(-c5ccccc5)oc3-4)c3ccc4c(c3)sc3ccccc34)cc2)cc1 ; c1ccc(-c2ccc(N(c3cc4c5c(cccc5c3)-c3nc(-c5ccccc5)oc3-4)c3cccc4c3sc3ccccc34)cc2)cc1 ; c1ccc(-c2ccc(N(c3ccc(-c4nc5c(o4)-c4cccc6cccc-5c46)cc3)c3cccc4sc5ccccc5c34)cc2)cc1]

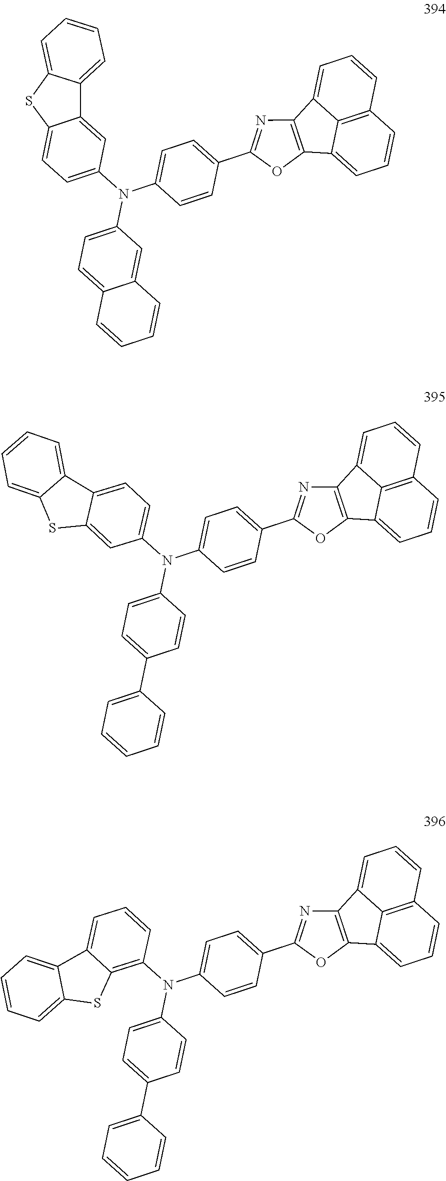 [SMILES: c1ccc2cc(N(c3ccc(-c4nc5c(o4)-c4cccc6cccc-5c46)cc3)c3ccc4sc5ccccc5c4c3)ccc2c1 ; c1ccc(-c2ccc(N(c3ccc(-c4nc5c(o4)-c4cccc6cccc-5c46)cc3)c3ccc4c(c3)sc3ccccc34)cc2)cc1 ; c1ccc(-c2ccc(N(c3ccc(-c4nc5c(o4)-c4cccc6cccc-5c46)cc3)c3cccc4c3sc3ccccc34)cc2)cc1]

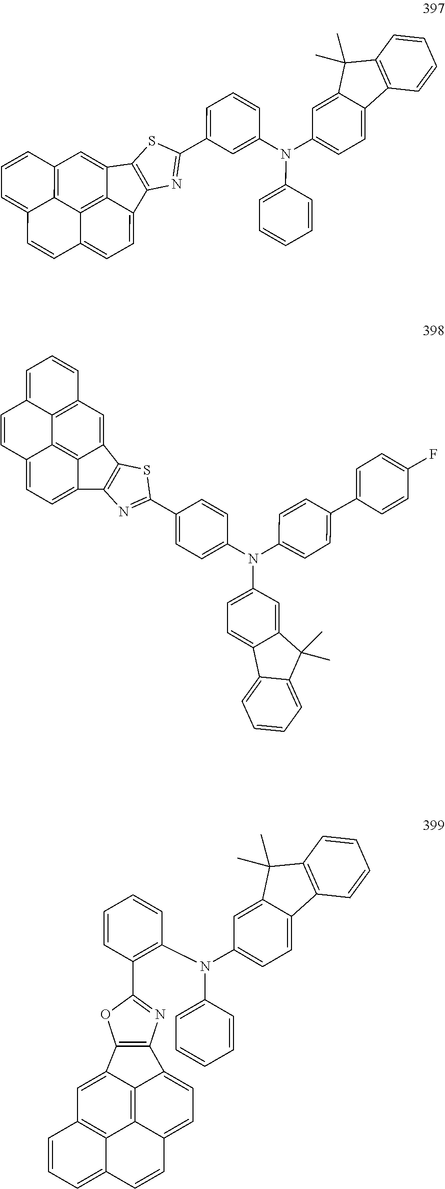 [SMILES: CC1(C)c2ccccc2-c2ccc(N(c3ccccc3)c3cccc(-c4nc5c(s4)-c4cc6cccc7ccc8ccc-5c4c8c76)c3)cc21 ; CC1(C)c2ccccc2-c2ccc(N(c3ccc(-c4ccc(F)cc4)cc3)c3ccc(-c4nc5c(s4)-c4cc6cccc7ccc8ccc-5c4c8c76)cc3)cc21 ; CC1(C)c2ccccc2-c2ccc(N(c3ccccc3)c3ccccc3-c3nc4c(o3)-c3cc5cccc6ccc7ccc-4c3c7c65)cc21]

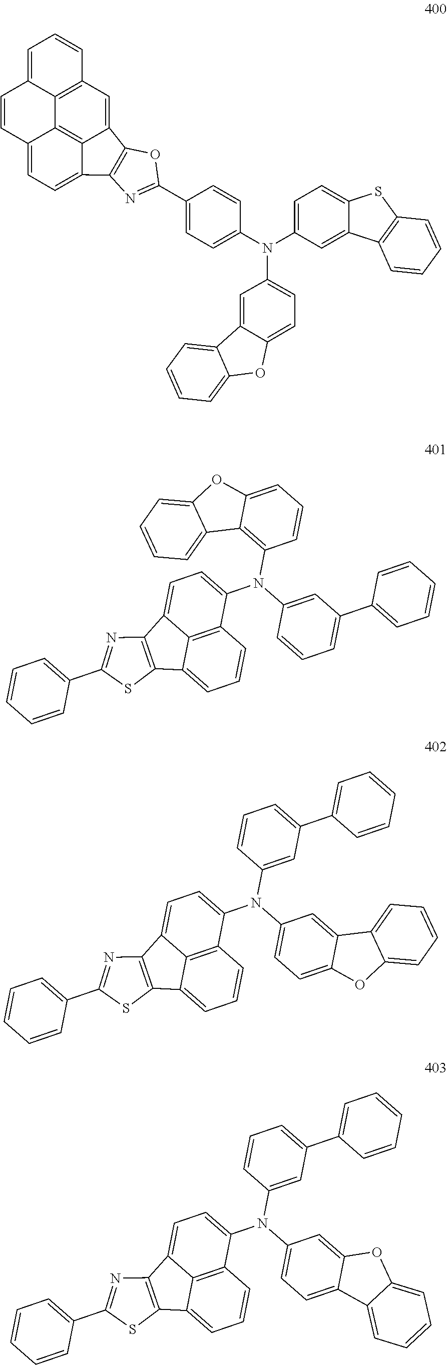 [SMILES: c1cc2ccc3ccc4c5c(cc(c1)c2c35)-c1oc(-c2ccc(N(c3ccc5oc6ccccc6c5c3)c3ccc5sc6ccccc6c5c3)cc2)nc1-4 ; c1ccc(-c2cccc(N(c3ccc4c5c(cccc35)-c3sc(-c5ccccc5)nc3-4)c3cccc4oc5ccccc5c34)c2)cc1 ; c1ccc(-c2cccc(N(c3ccc4oc5ccccc5c4c3)c3ccc4c5c(cccc35)-c3sc(-c5ccccc5)nc3-4)c2)cc1 ; c1ccc(-c2cccc(N(c3ccc4c(c3)oc3ccccc34)c3ccc4c5c(cccc35)-c3sc(-c5ccccc5)nc3-4)c2)cc1]

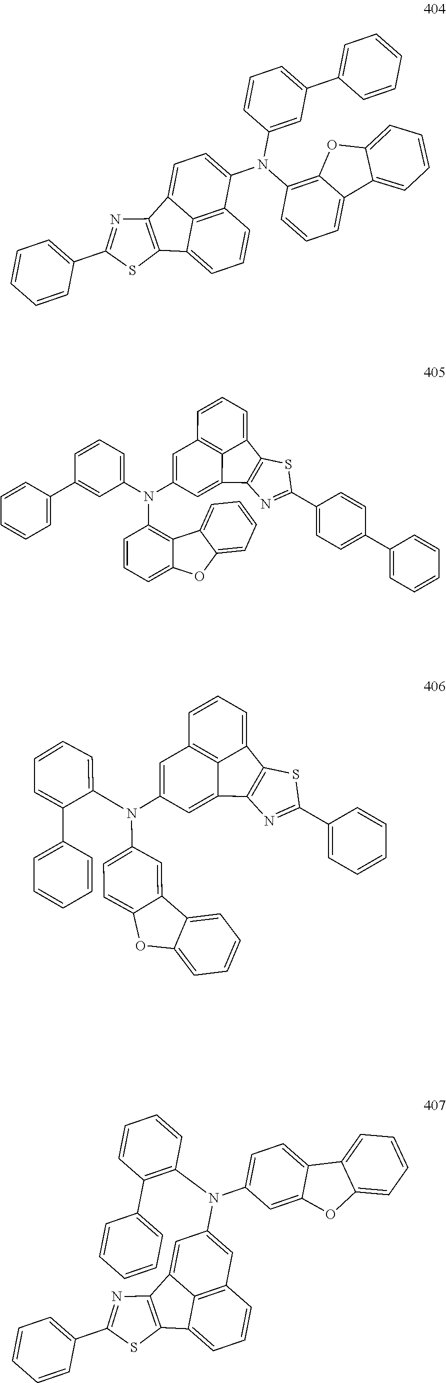 [SMILES: c1ccc(-c2cccc(N(c3ccc4c5c(cccc35)-c3sc(-c5ccccc5)nc3-4)c3cccc4c3oc3ccccc34)c2)cc1 ; c1ccc(-c2ccc(-c3nc4c(s3)-c3cccc5cc(N(c6cccc(-c7ccccc7)c6)c6cccc7oc8ccccc8c67)cc-4c35)cc2)cc1 ; c1ccc(-c2nc3c(s2)-c2cccc4cc(N(c5ccc6oc7ccccc7c6c5)c5ccccc5-c5ccccc5)cc-3c24)cc1 ; c1ccc(-c2nc3c(s2)-c2cccc4cc(N(c5ccc6c(c5)oc5ccccc56)c5ccccc5-c5ccccc5)cc-3c24)cc1]

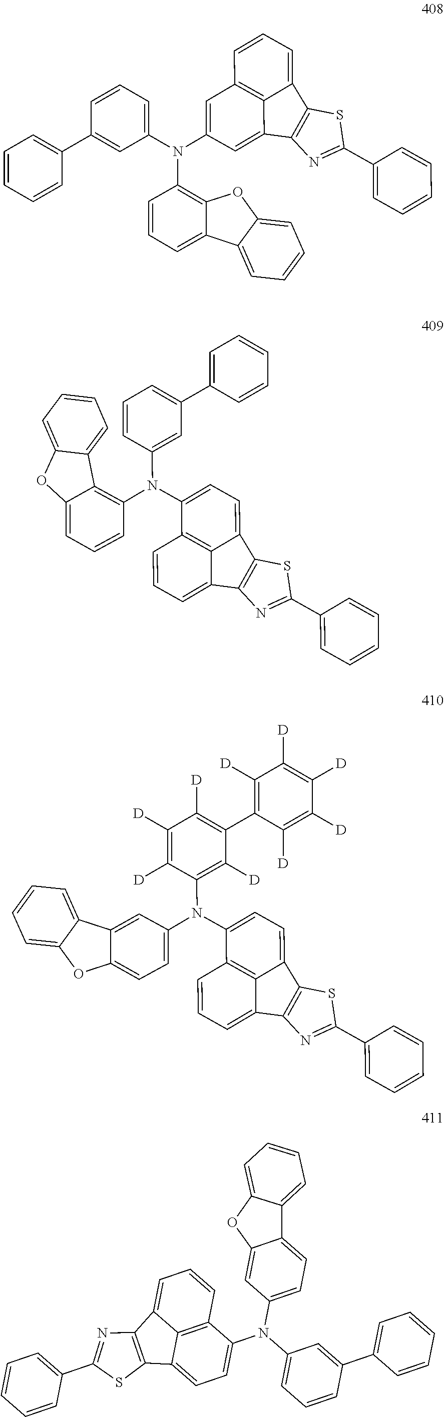 [SMILES: c1ccc(-c2cccc(N(c3cc4c5c(cccc5c3)-c3sc(-c5ccccc5)nc3-4)c3cccc4c3oc3ccccc34)c2)cc1 ; c1ccc(-c2cccc(N(c3ccc4c5c(cccc35)-c3nc(-c5ccccc5)sc3-4)c3cccc4oc5ccccc5c34)c2)cc1 ; [2H]c1c([2H])c([2H])c(-c2c([2H])c([2H])c([2H])c(N(c3ccc4oc5ccccc5c4c3)c3ccc4c5c(cccc35)-c3nc(-c5ccccc5)sc3-4)c2[2H])c([2H])c1[2H] ; c1ccc(-c2cccc(N(c3ccc4c(c3)oc3ccccc34)c3ccc4c5c(cccc35)-c3nc(-c5ccccc5)sc3-4)c2)cc1]

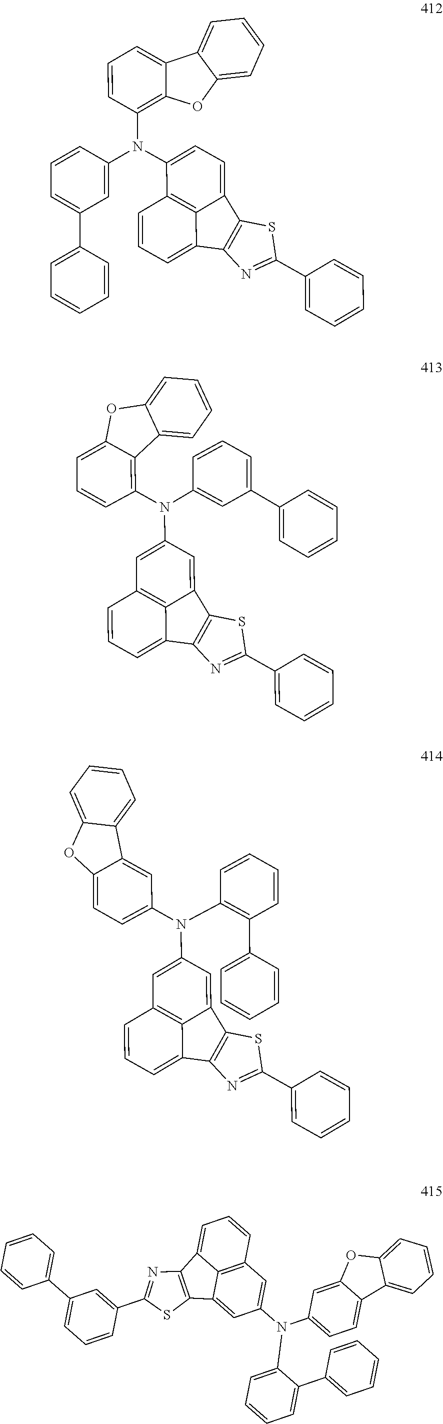 [SMILES: c1ccc(-c2cccc(N(c3ccc4c5c(cccc35)-c3nc(-c5ccccc5)sc3-4)c3cccc4c3oc3ccccc34)c2)cc1 ; c1ccc(-c2cccc(N(c3cc4c5c(cccc5c3)-c3nc(-c5ccccc5)sc3-4)c3cccc4oc5ccccc5c34)c2)cc1 ; c1ccc(-c2nc3c(s2)-c2cc(N(c4ccc5oc6ccccc6c5c4)c4ccccc4-c4ccccc4)cc4cccc-3c24)cc1 ; c1ccc(-c2cccc(-c3nc4c(s3)-c3cc(N(c5ccc6c(c5)oc5ccccc56)c5ccccc5-c5ccccc5)cc5cccc-4c35)c2)cc1]

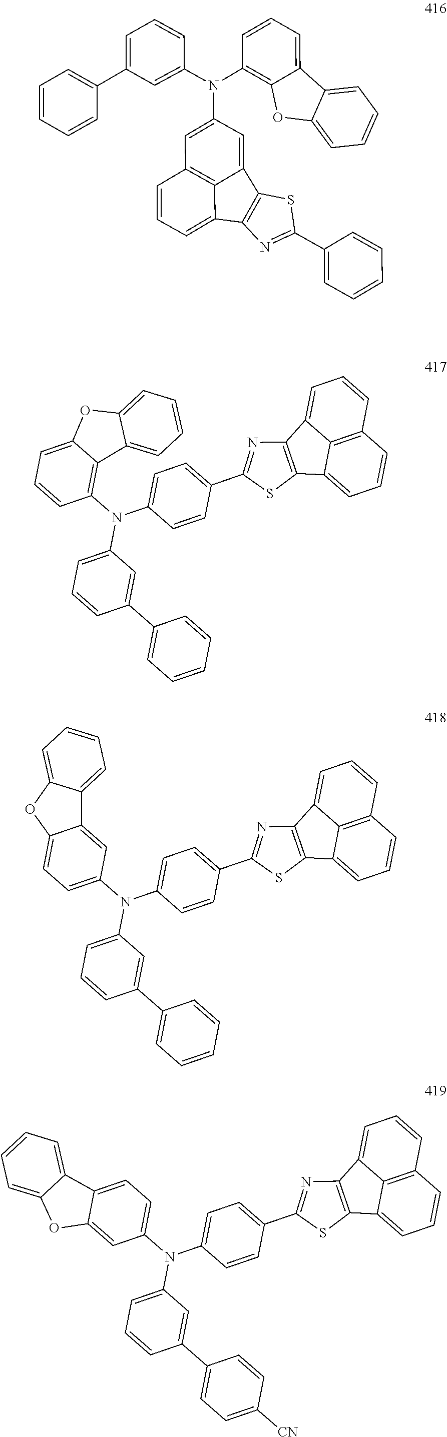 [SMILES: c1ccc(-c2cccc(N(c3cc4c5c(cccc5c3)-c3nc(-c5ccccc5)sc3-4)c3cccc4c3oc3ccccc34)c2)cc1 ; c1ccc(-c2cccc(N(c3ccc(-c4nc5c(s4)-c4cccc6cccc-5c46)cc3)c3cccc4oc5ccccc5c34)c2)cc1 ; c1ccc(-c2cccc(N(c3ccc(-c4nc5c(s4)-c4cccc6cccc-5c46)cc3)c3ccc4oc5ccccc5c4c3)c2)cc1 ; N#Cc1ccc(-c2cccc(N(c3ccc(-c4nc5c(s4)-c4cccc6cccc-5c46)cc3)c3ccc4c(c3)oc3ccccc34)c2)cc1]

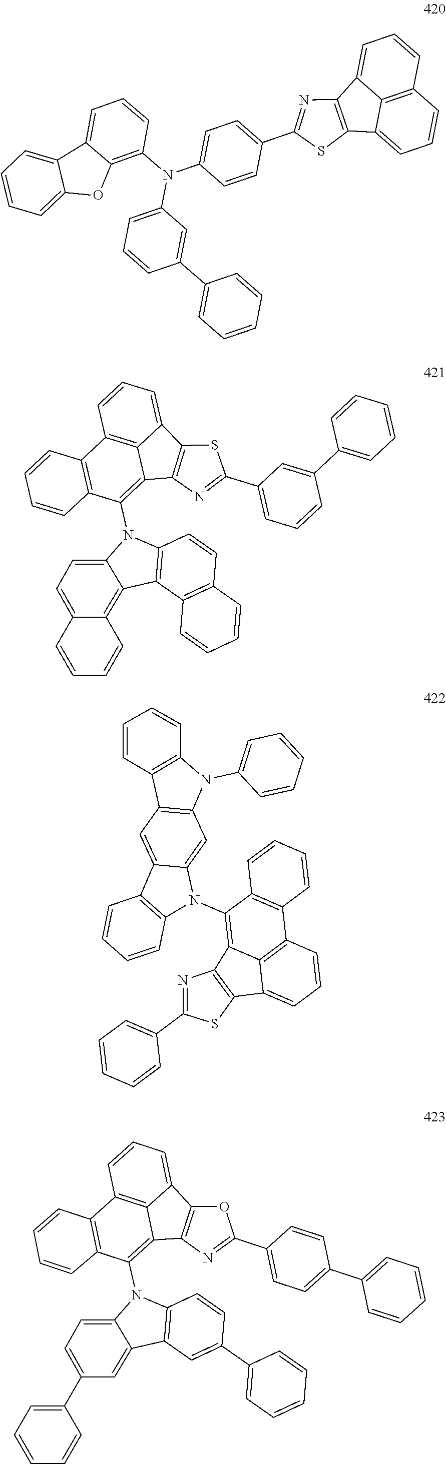 [SMILES: c1ccc(-c2cccc(N(c3ccc(-c4nc5c(s4)-c4cccc6cccc-5c46)cc3)c3cccc4c3oc3ccccc34)c2)cc1 ; c1ccc(-c2cccc(-c3nc4c(s3)-c3cccc5c3c-4c(-n3c4ccc6ccccc6c4c4c6ccccc6ccc43)c3ccccc35)c2)cc1 ; c1ccc(-c2nc3c(s2)-c2cccc4c2c-3c(-n2c3ccccc3c3cc5c6ccccc6n(-c6ccccc6)c5cc32)c2ccccc24)cc1 ; c1ccc(-c2ccc(-c3nc4c(o3)-c3cccc5c3c-4c(-n3c4ccc(-c6ccccc6)cc4c4cc(-c6ccccc6)ccc43)c3ccccc35)cc2)cc1]

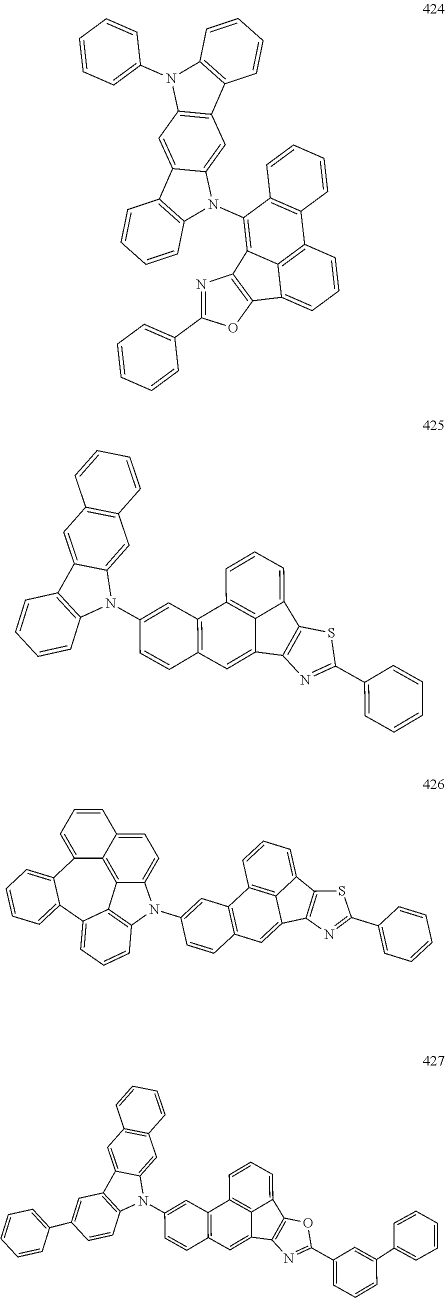 [SMILES: c1ccc(-c2nc3c(o2)-c2cccc4c2c-3c(-n2c3ccccc3c3cc5c(cc32)c2ccccc2n5-c2ccccc2)c2ccccc24)cc1 ; c1ccc(-c2nc3c(s2)-c2cccc4c2c-3cc2ccc(-n3c5ccccc5c5cc6ccccc6cc53)cc24)cc1 ; c1ccc(-c2nc3c(s2)-c2cccc4c2c-3cc2ccc(-n3c5cccc6c5c5c7c(cccc7ccc53)-c3ccccc3-6)cc24)cc1 ; c1ccc(-c2cccc(-c3nc4c(o3)-c3cccc5c3c-4cc3ccc(-n4c6ccc(-c7ccccc7)cc6c6cc7ccccc7cc64)cc35)c2)cc1]

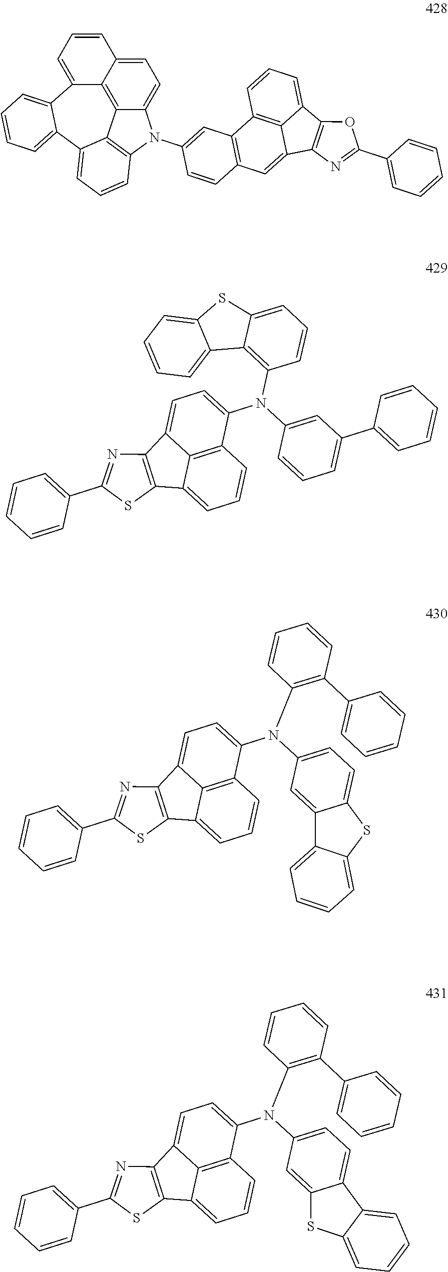 [SMILES: c1ccc(-c2nc3c(o2)-c2cccc4c2c-3cc2ccc(-n3c5cccc6c5c5c7c(cccc7ccc53)-c3ccccc3-6)cc24)cc1 ; c1ccc(-c2cccc(N(c3ccc4c5c(cccc35)-c3sc(-c5ccccc5)nc3-4)c3cccc4sc5ccccc5c34)c2)cc1 ; c1ccc(-c2nc3c(s2)-c2cccc4c(N(c5ccc6sc7ccccc7c6c5)c5ccccc5-c5ccccc5)ccc-3c24)cc1 ; c1ccc(-c2nc3c(s2)-c2cccc4c(N(c5ccc6c(c5)sc5ccccc56)c5ccccc5-c5ccccc5)ccc-3c24)cc1]

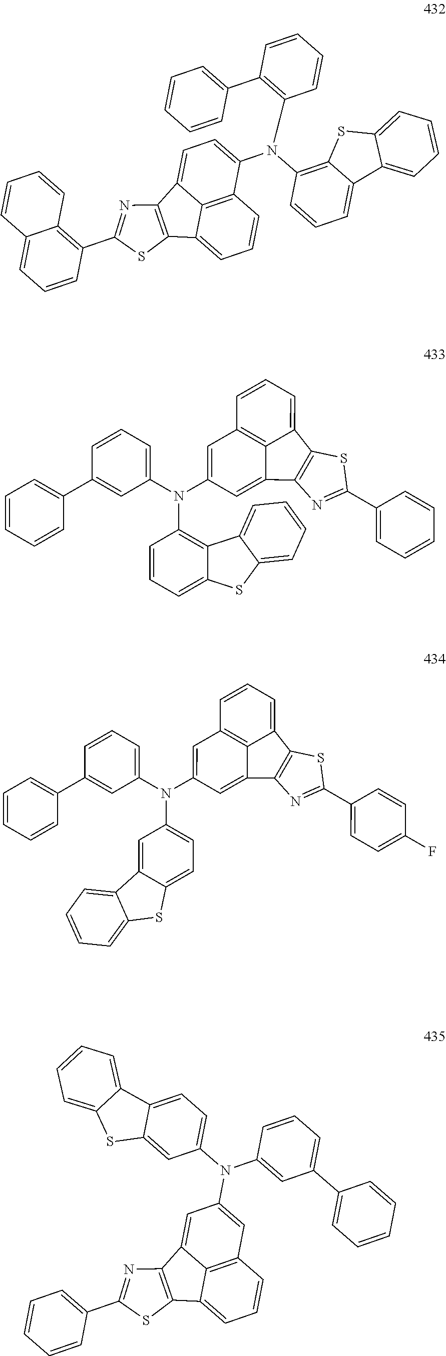 [SMILES: c1ccc(-c2ccccc2N(c2ccc3c4c(cccc24)-c2sc(-c4cccc5ccccc45)nc2-3)c2cccc3c2sc2ccccc23)cc1 ; c1ccc(-c2cccc(N(c3cc4c5c(cccc5c3)-c3sc(-c5ccccc5)nc3-4)c3cccc4sc5ccccc5c34)c2)cc1 ; Fc1ccc(-c2nc3c(s2)-c2cccc4cc(N(c5cccc(-c6ccccc6)c5)c5ccc6sc7ccccc7c6c5)cc-3c24)cc1 ; c1ccc(-c2cccc(N(c3cc4c5c(cccc5c3)-c3sc(-c5ccccc5)nc3-4)c3ccc4c(c3)sc3ccccc34)c2)cc1]

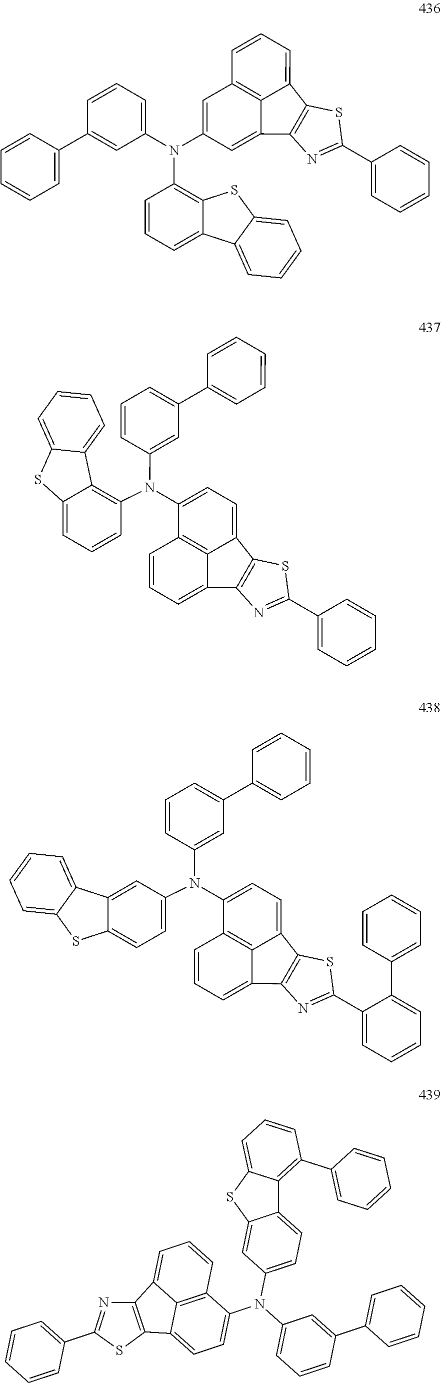 [SMILES: c1ccc(-c2cccc(N(c3cc4c5c(cccc5c3)-c3sc(-c5ccccc5)nc3-4)c3cccc4c3sc3ccccc34)c2)cc1 ; c1ccc(-c2cccc(N(c3ccc4c5c(cccc35)-c3nc(-c5ccccc5)sc3-4)c3cccc4sc5ccccc5c34)c2)cc1 ; c1ccc(-c2cccc(N(c3ccc4sc5ccccc5c4c3)c3ccc4c5c(cccc35)-c3nc(-c5ccccc5-c5ccccc5)sc3-4)c2)cc1 ; c1ccc(-c2cccc(N(c3ccc4c(c3)sc3cccc(-c5ccccc5)c34)c3ccc4c5c(cccc35)-c3nc(-c5ccccc5)sc3-4)c2)cc1]

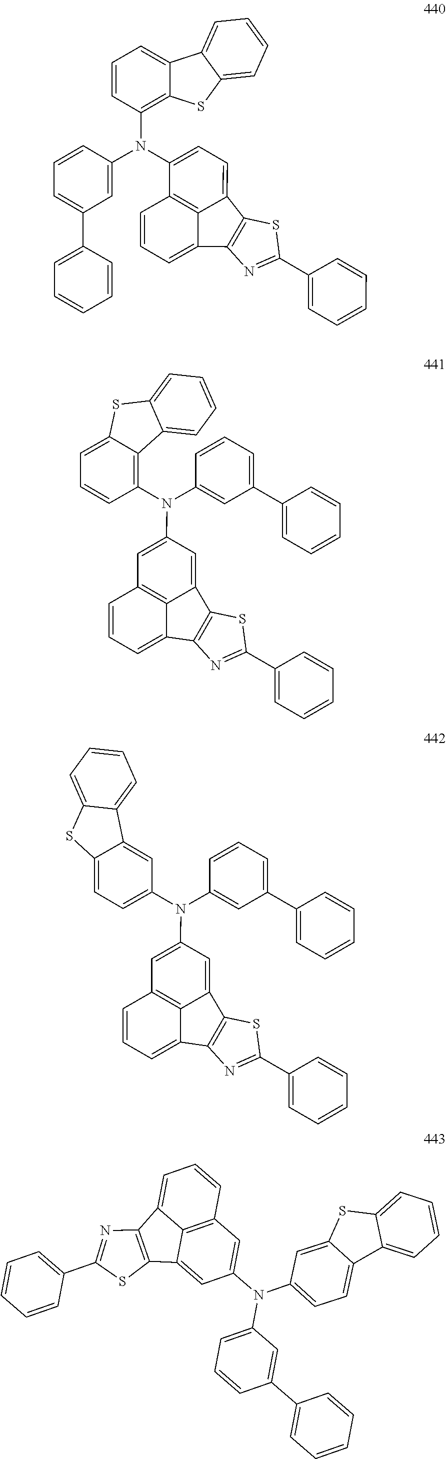 [SMILES: c1ccc(-c2cccc(N(c3ccc4c5c(cccc35)-c3nc(-c5ccccc5)sc3-4)c3cccc4c3sc3ccccc34)c2)cc1 ; c1ccc(-c2cccc(N(c3cc4c5c(cccc5c3)-c3nc(-c5ccccc5)sc3-4)c3cccc4sc5ccccc5c34)c2)cc1 ; c1ccc(-c2cccc(N(c3cc4c5c(cccc5c3)-c3nc(-c5ccccc5)sc3-4)c3ccc4sc5ccccc5c4c3)c2)cc1 ; c1ccc(-c2cccc(N(c3cc4c5c(cccc5c3)-c3nc(-c5ccccc5)sc3-4)c3ccc4c(c3)sc3ccccc34)c2)cc1]

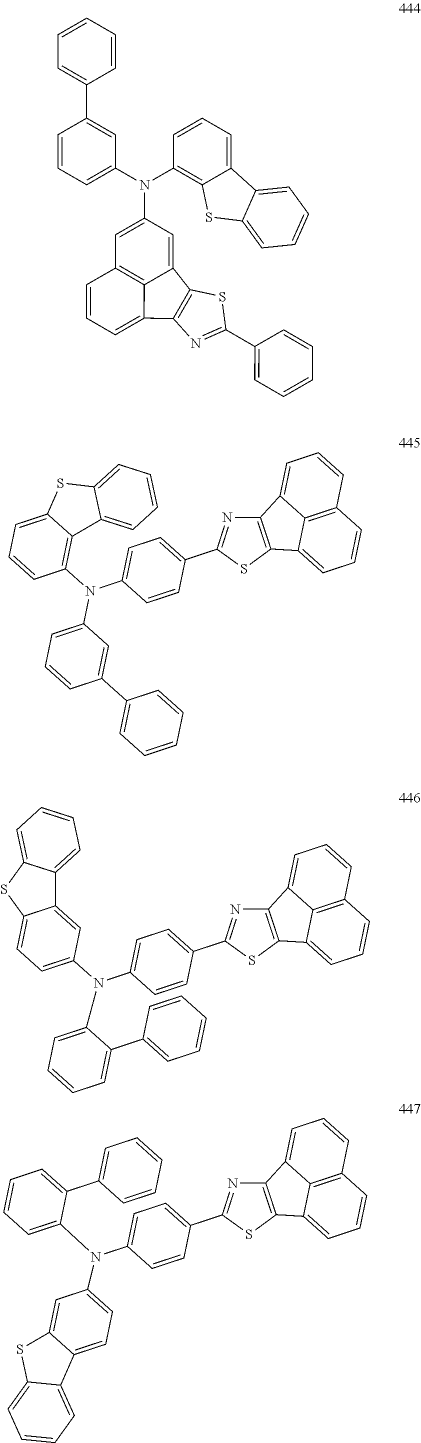 [SMILES: c1ccc(-c2cccc(N(c3cc4c5c(cccc5c3)-c3nc(-c5ccccc5)sc3-4)c3cccc4c3sc3ccccc34)c2)cc1 ; c1ccc(-c2cccc(N(c3ccc(-c4nc5c(s4)-c4cccc6cccc-5c46)cc3)c3cccc4sc5ccccc5c34)c2)cc1 ; c1ccc(-c2ccccc2N(c2ccc(-c3nc4c(s3)-c3cccc5cccc-4c35)cc2)c2ccc3sc4ccccc4c3c2)cc1 ; c1ccc(-c2ccccc2N(c2ccc(-c3nc4c(s3)-c3cccc5cccc-4c35)cc2)c2ccc3c(c2)sc2ccccc23)cc1]

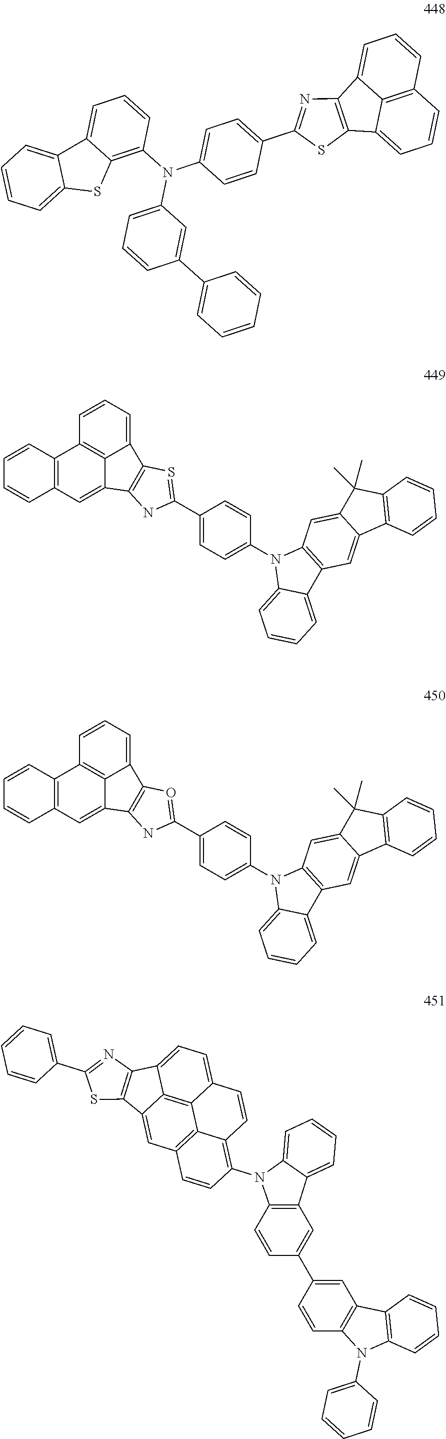

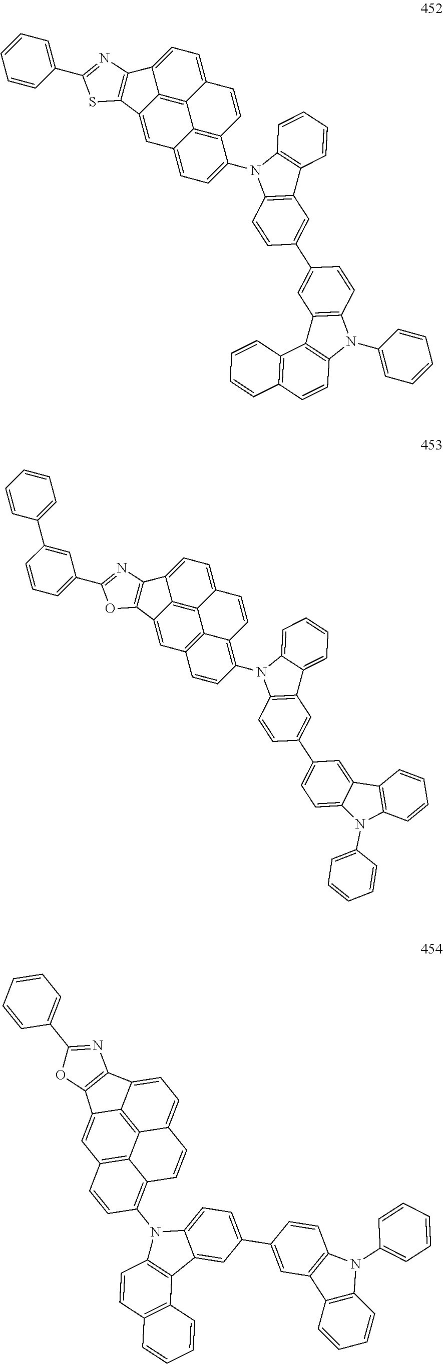 [SMILES: c1ccc(-c2nc3c(s2)-c2cc4ccc(-n5c6ccccc6c6cc(-c7ccc8c(c7)c7c9ccccc9ccc7n8-c7ccccc7)ccc65)c5ccc6ccc-3c2c6c45)cc1 ; c1ccc(-c2cccc(-c3nc4c(o3)-c3cc5ccc(-n6c7ccccc7c7cc(-c8ccc9c(c8)c8ccccc8n9-c8ccccc8)ccc76)c6ccc7ccc-4c3c7c56)c2)cc1 ; c1ccc(-c2nc3c(o2)-c2cc4ccc(-n5c6ccc(-c7ccc8c(c7)c7ccccc7n8-c7ccccc7)cc6c6c7ccccc7ccc65)c5ccc6ccc-3c2c6c45)cc1]

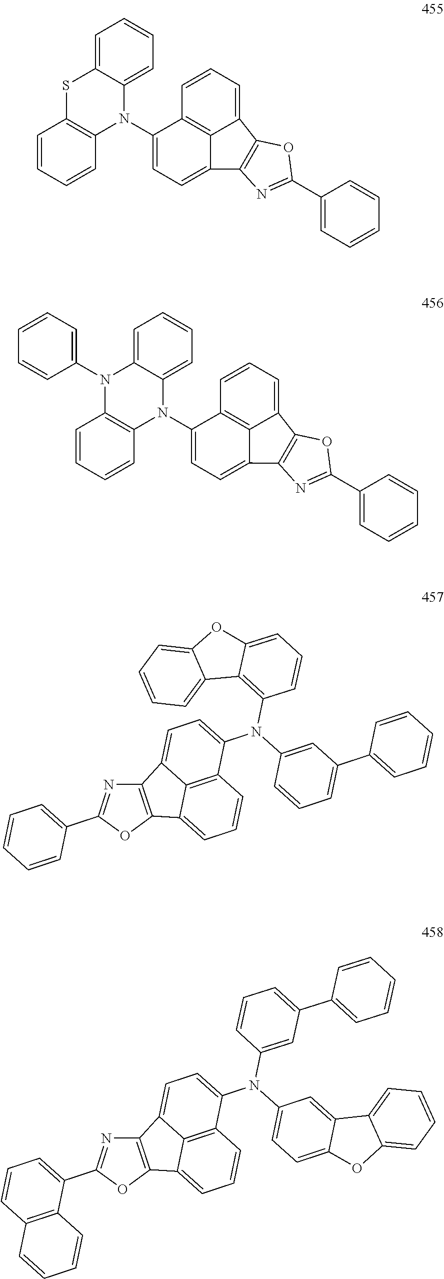 [SMILES: c1ccc(-c2nc3c(o2)-c2cccc4c(N5c6ccccc6Sc6ccccc65)ccc-3c24)cc1 ; c1ccc(-c2nc3c(o2)-c2cccc4c(N5c6ccccc6N(c6ccccc6)c6ccccc65)ccc-3c24)cc1 ; c1ccc(-c2cccc(N(c3ccc4c5c(cccc35)-c3oc(-c5ccccc5)nc3-4)c3cccc4oc5ccccc5c34)c2)cc1 ; c1ccc(-c2cccc(N(c3ccc4oc5ccccc5c4c3)c3ccc4c5c(cccc35)-c3oc(-c5cccc6ccccc56)nc3-4)c2)cc1]

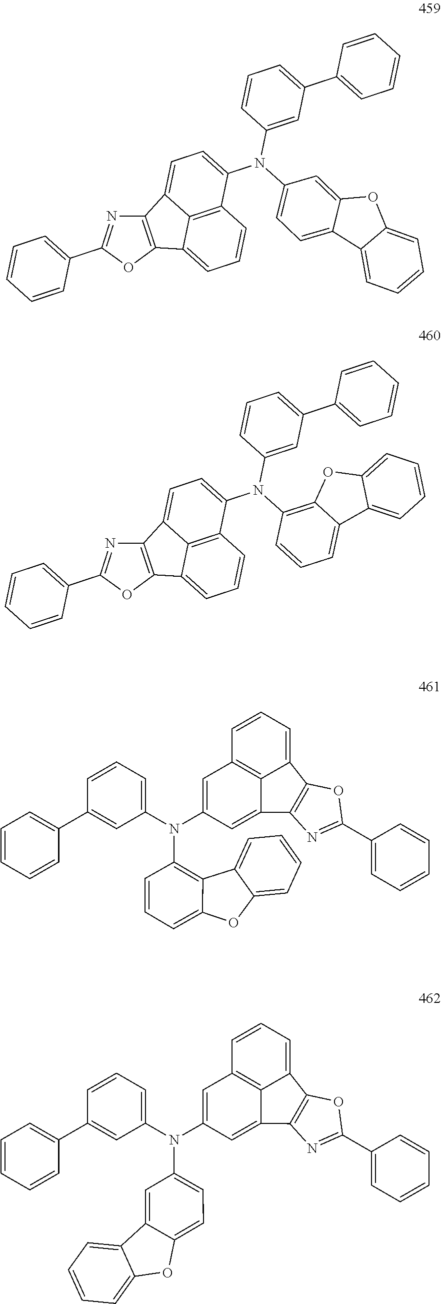 [SMILES: c1ccc(-c2cccc(N(c3ccc4c(c3)oc3ccccc34)c3ccc4c5c(cccc35)-c3oc(-c5ccccc5)nc3-4)c2)cc1 ; c1ccc(-c2cccc(N(c3ccc4c5c(cccc35)-c3oc(-c5ccccc5)nc3-4)c3cccc4c3oc3ccccc34)c2)cc1 ; c1ccc(-c2cccc(N(c3cc4c5c(cccc5c3)-c3oc(-c5ccccc5)nc3-4)c3cccc4oc5ccccc5c34)c2)cc1 ; c1ccc(-c2cccc(N(c3cc4c5c(cccc5c3)-c3oc(-c5ccccc5)nc3-4)c3ccc4oc5ccccc5c4c3)c2)cc1]

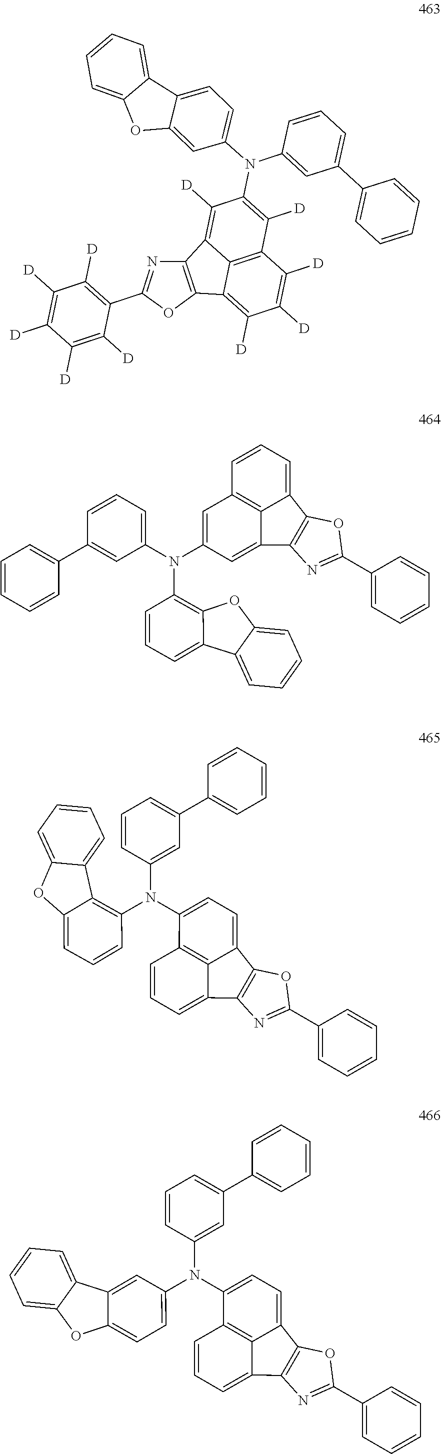 [SMILES: [2H]c1c([2H])c([2H])c(-c2nc3c(o2)-c2c([2H])c([2H])c([2H])c4c([2H])c(N(c5cccc(-c6ccccc6)c5)c5ccc6c(c5)oc5ccccc56)c([2H])c-3c24)c([2H])c1[2H] ; c1ccc(-c2cccc(N(c3cc4c5c(cccc5c3)-c3oc(-c5ccccc5)nc3-4)c3cccc4c3oc3ccccc34)c2)cc1 ; c1ccc(-c2cccc(N(c3ccc4c5c(cccc35)-c3nc(-c5ccccc5)oc3-4)c3cccc4oc5ccccc5c34)c2)cc1 ; c1ccc(-c2cccc(N(c3ccc4oc5ccccc5c4c3)c3ccc4c5c(cccc35)-c3nc(-c5ccccc5)oc3-4)c2)cc1]

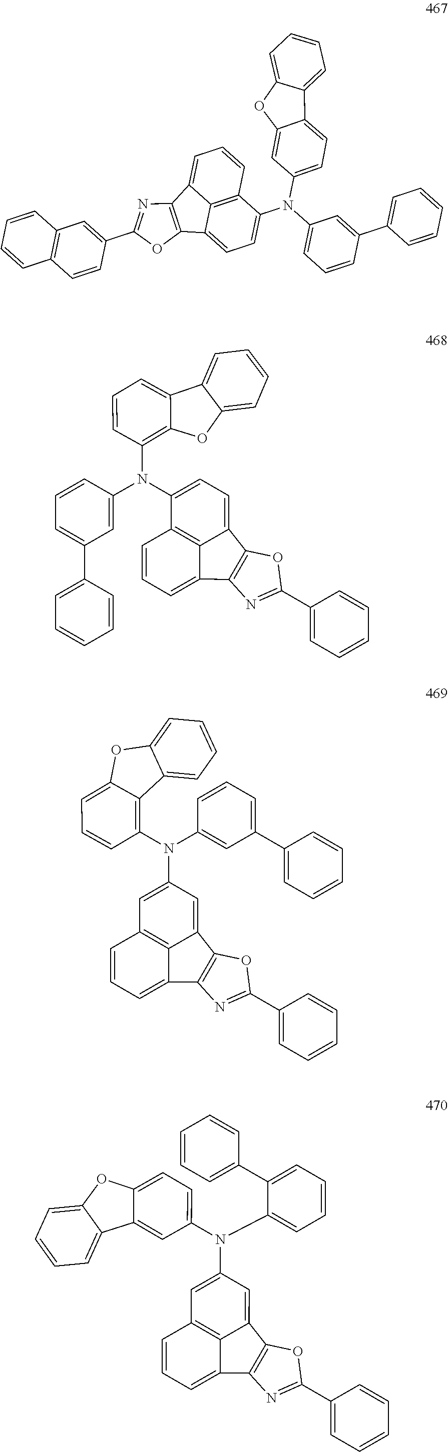 [SMILES: c1ccc(-c2cccc(N(c3ccc4c(c3)oc3ccccc34)c3ccc4c5c(cccc35)-c3nc(-c5ccc6ccccc6c5)oc3-4)c2)cc1 ; c1ccc(-c2cccc(N(c3ccc4c5c(cccc35)-c3nc(-c5ccccc5)oc3-4)c3cccc4c3oc3ccccc34)c2)cc1 ; c1ccc(-c2cccc(N(c3cc4c5c(cccc5c3)-c3nc(-c5ccccc5)oc3-4)c3cccc4oc5ccccc5c34)c2)cc1 ; c1ccc(-c2nc3c(o2)-c2cc(N(c4ccc5oc6ccccc6c5c4)c4ccccc4-c4ccccc4)cc4cccc-3c24)cc1]

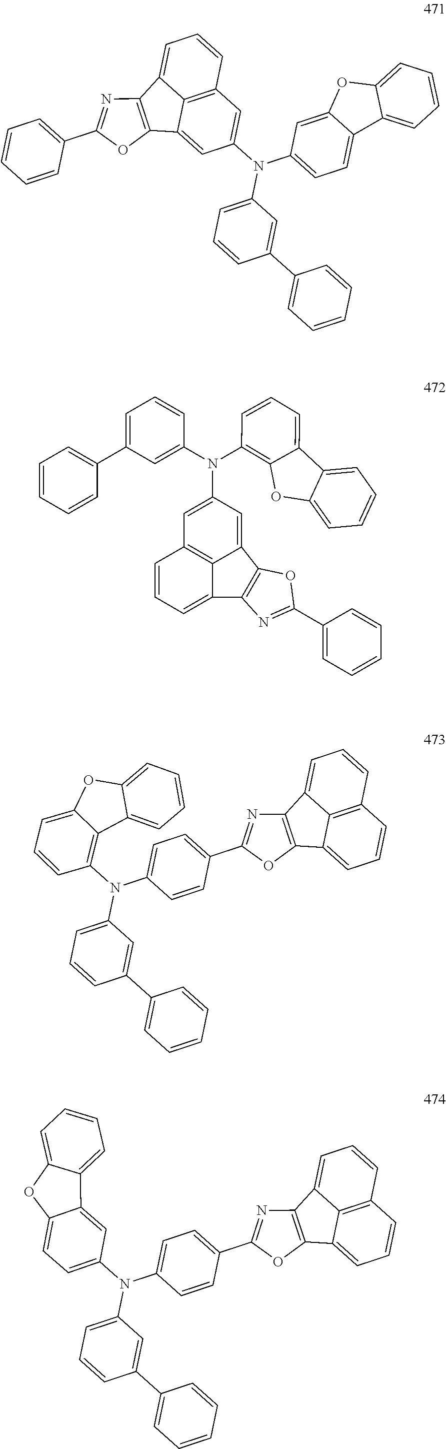 [SMILES: c1ccc(-c2cccc(N(c3cc4c5c(cccc5c3)-c3nc(-c5ccccc5)oc3-4)c3ccc4c(c3)oc3ccccc34)c2)cc1 ; c1ccc(-c2cccc(N(c3cc4c5c(cccc5c3)-c3nc(-c5ccccc5)oc3-4)c3cccc4c3oc3ccccc34)c2)cc1 ; c1ccc(-c2cccc(N(c3ccc(-c4nc5c(o4)-c4cccc6cccc-5c46)cc3)c3cccc4oc5ccccc5c34)c2)cc1 ; c1ccc(-c2cccc(N(c3ccc(-c4nc5c(o4)-c4cccc6cccc-5c46)cc3)c3ccc4oc5ccccc5c4c3)c2)cc1]

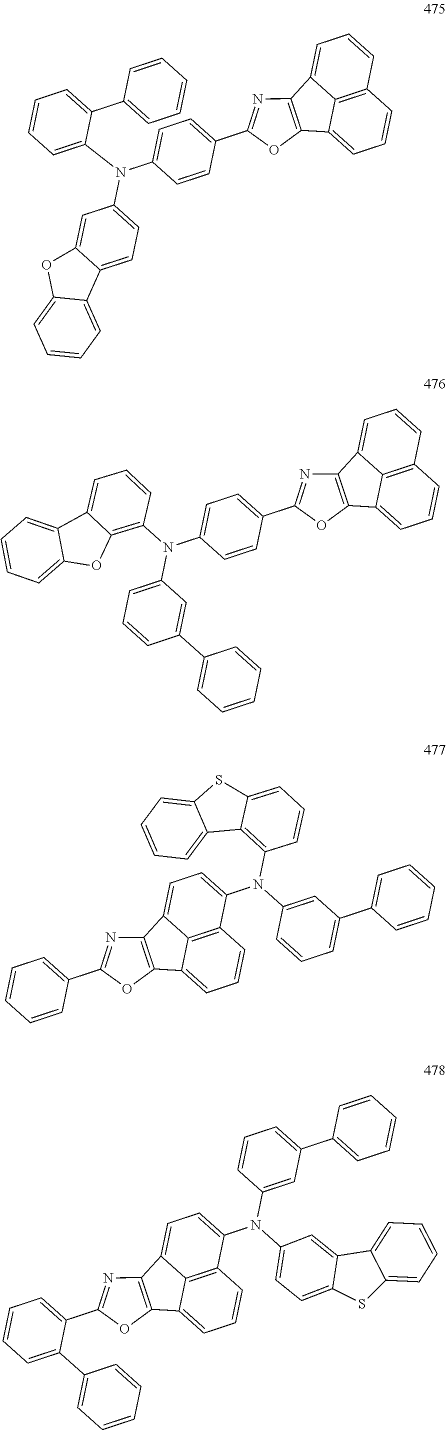 [SMILES: c1ccc(-c2ccccc2N(c2ccc(-c3nc4c(o3)-c3cccc5cccc-4c35)cc2)c2ccc3c(c2)oc2ccccc23)cc1 ; c1ccc(-c2cccc(N(c3ccc(-c4nc5c(o4)-c4cccc6cccc-5c46)cc3)c3cccc4c3oc3ccccc34)c2)cc1 ; c1ccc(-c2cccc(N(c3ccc4c5c(cccc35)-c3oc(-c5ccccc5)nc3-4)c3cccc4sc5ccccc5c34)c2)cc1 ; c1ccc(-c2cccc(N(c3ccc4sc5ccccc5c4c3)c3ccc4c5c(cccc35)-c3oc(-c5ccccc5-c5ccccc5)nc3-4)c2)cc1]

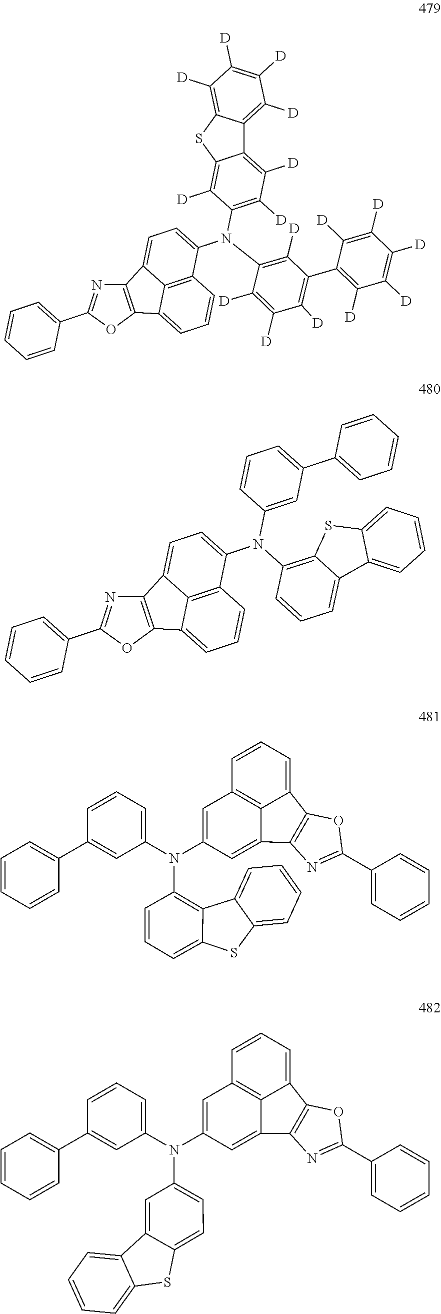 [SMILES: [2H]c1c([2H])c([2H])c(-c2c([2H])c([2H])c([2H])c(N(c3c([2H])c([2H])c4c(sc5c([2H])c([2H])c([2H])c([2H])c54)c3[2H])c3ccc4c5c(cccc35)-c3oc(-c5ccccc5)nc3-4)c2[2H])c([2H])c1[2H] ; c1ccc(-c2cccc(N(c3ccc4c5c(cccc35)-c3oc(-c5ccccc5)nc3-4)c3cccc4c3sc3ccccc34)c2)cc1 ; c1ccc(-c2cccc(N(c3cc4c5c(cccc5c3)-c3oc(-c5ccccc5)nc3-4)c3cccc4sc5ccccc5c34)c2)cc1 ; c1ccc(-c2cccc(N(c3cc4c5c(cccc5c3)-c3oc(-c5ccccc5)nc3-4)c3ccc4sc5ccccc5c4c3)c2)cc1]

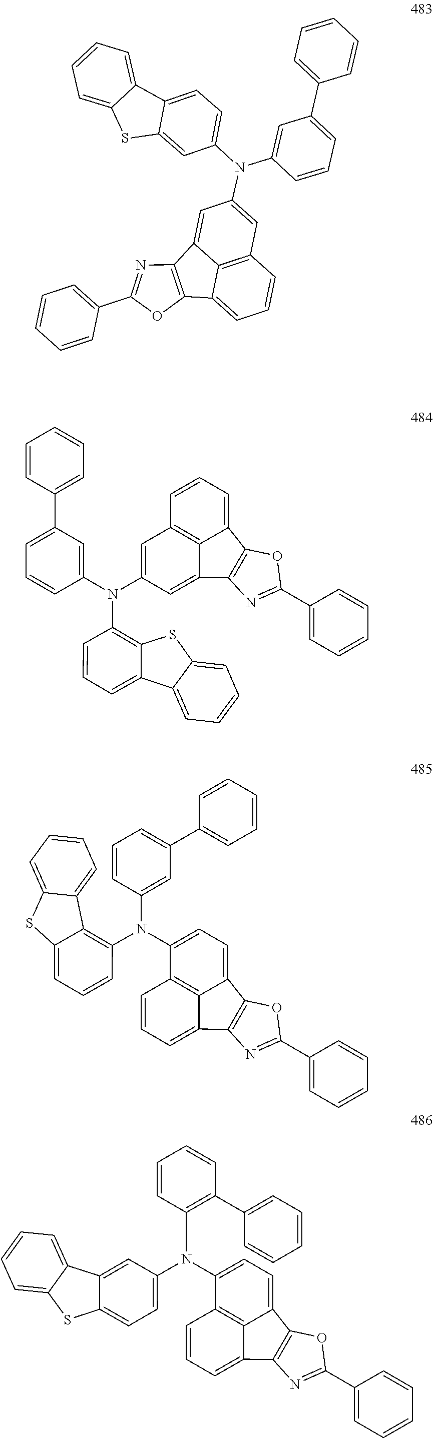 [SMILES: c1ccc(-c2cccc(N(c3cc4c5c(cccc5c3)-c3oc(-c5ccccc5)nc3-4)c3ccc4c(c3)sc3ccccc34)c2)cc1 ; c1ccc(-c2cccc(N(c3cc4c5c(cccc5c3)-c3oc(-c5ccccc5)nc3-4)c3cccc4c3sc3ccccc34)c2)cc1 ; c1ccc(-c2cccc(N(c3ccc4c5c(cccc35)-c3nc(-c5ccccc5)oc3-4)c3cccc4sc5ccccc5c34)c2)cc1 ; c1ccc(-c2nc3c(o2)-c2ccc(N(c4ccc5sc6ccccc6c5c4)c4ccccc4-c4ccccc4)c4cccc-3c24)cc1]

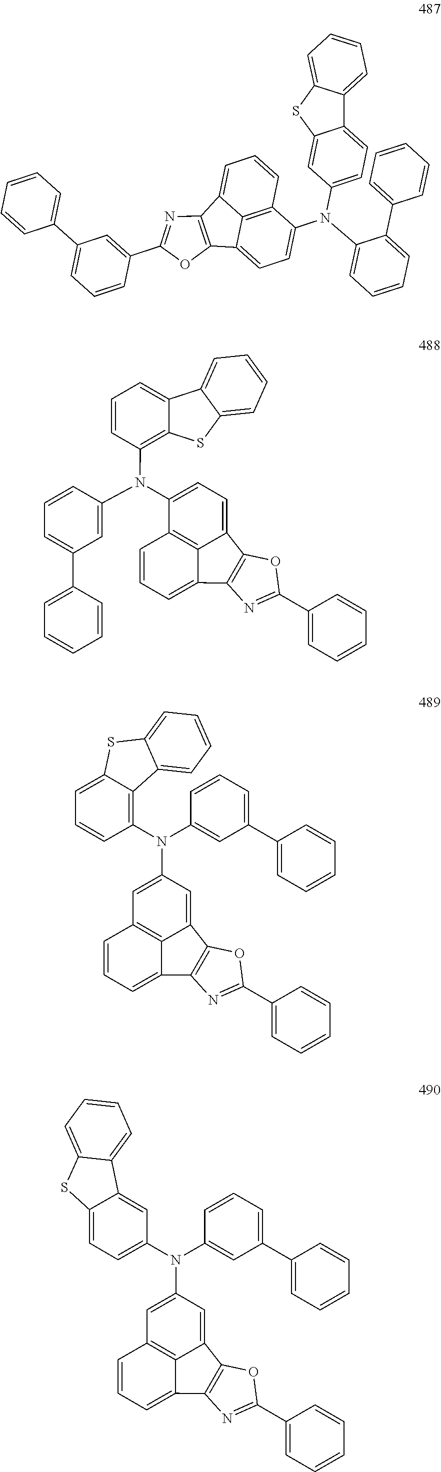 [SMILES: c1ccc(-c2cccc(-c3nc4c(o3)-c3ccc(N(c5ccc6c(c5)sc5ccccc56)c5ccccc5-c5ccccc5)c5cccc-4c35)c2)cc1 ; c1ccc(-c2cccc(N(c3ccc4c5c(cccc35)-c3nc(-c5ccccc5)oc3-4)c3cccc4c3sc3ccccc34)c2)cc1 ; c1ccc(-c2cccc(N(c3cc4c5c(cccc5c3)-c3nc(-c5ccccc5)oc3-4)c3cccc4sc5ccccc5c34)c2)cc1 ; c1ccc(-c2cccc(N(c3cc4c5c(cccc5c3)-c3nc(-c5ccccc5)oc3-4)c3ccc4sc5ccccc5c4c3)c2)cc1]

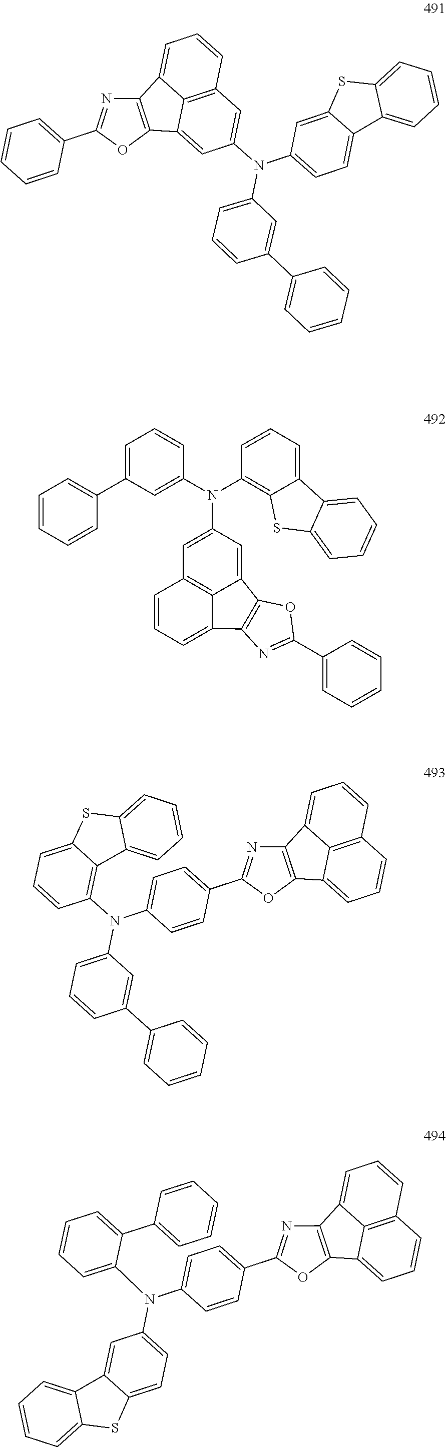 [SMILES: c1ccc(-c2cccc(N(c3cc4c5c(cccc5c3)-c3nc(-c5ccccc5)oc3-4)c3ccc4c(c3)sc3ccccc34)c2)cc1 ; c1ccc(-c2cccc(N(c3cc4c5c(cccc5c3)-c3nc(-c5ccccc5)oc3-4)c3cccc4c3sc3ccccc34)c2)cc1 ; c1ccc(-c2cccc(N(c3ccc(-c4nc5c(o4)-c4cccc6cccc-5c46)cc3)c3cccc4sc5ccccc5c34)c2)cc1 ; c1ccc(-c2ccccc2N(c2ccc(-c3nc4c(o3)-c3cccc5cccc-4c35)cc2)c2ccc3sc4ccccc4c3c2)cc1]

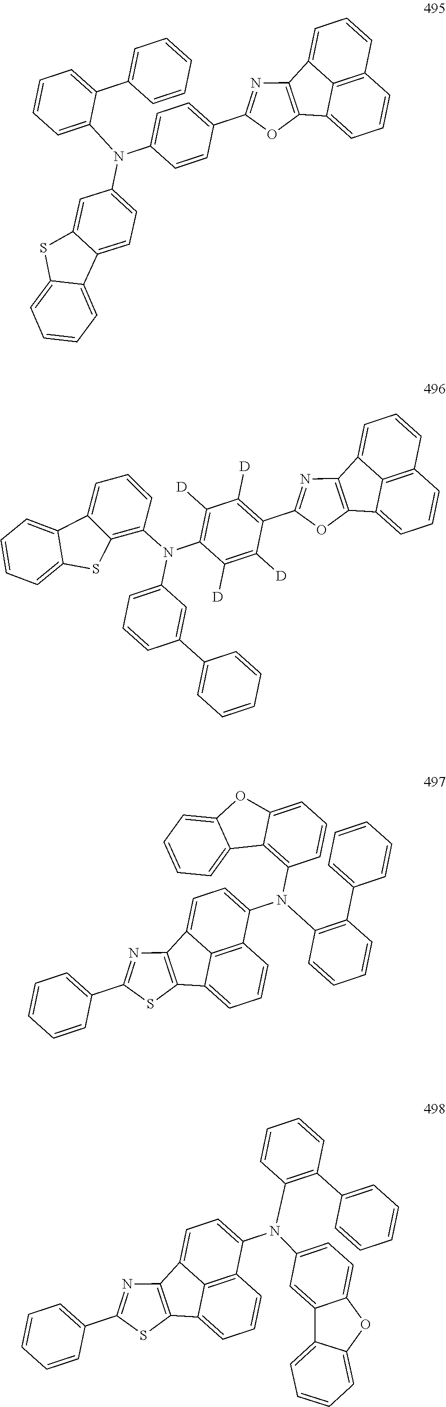 [SMILES: c1ccc(-c2ccccc2N(c2ccc(-c3nc4c(o3)-c3cccc5cccc-4c35)cc2)c2ccc3c(c2)sc2ccccc23)cc1 ; [2H]c1c([2H])c(N(c2cccc(-c3ccccc3)c2)c2cccc3c2sc2ccccc23)c([2H])c([2H])c1-c1nc2c(o1)-c1cccc3cccc-2c13 ; c1ccc(-c2nc3c(s2)-c2cccc4c(N(c5ccccc5-c5ccccc5)c5cccc6oc7ccccc7c56)ccc-3c24)cc1 ; c1ccc(-c2nc3c(s2)-c2cccc4c(N(c5ccc6oc7ccccc7c6c5)c5ccccc5-c5ccccc5)ccc-3c24)cc1]

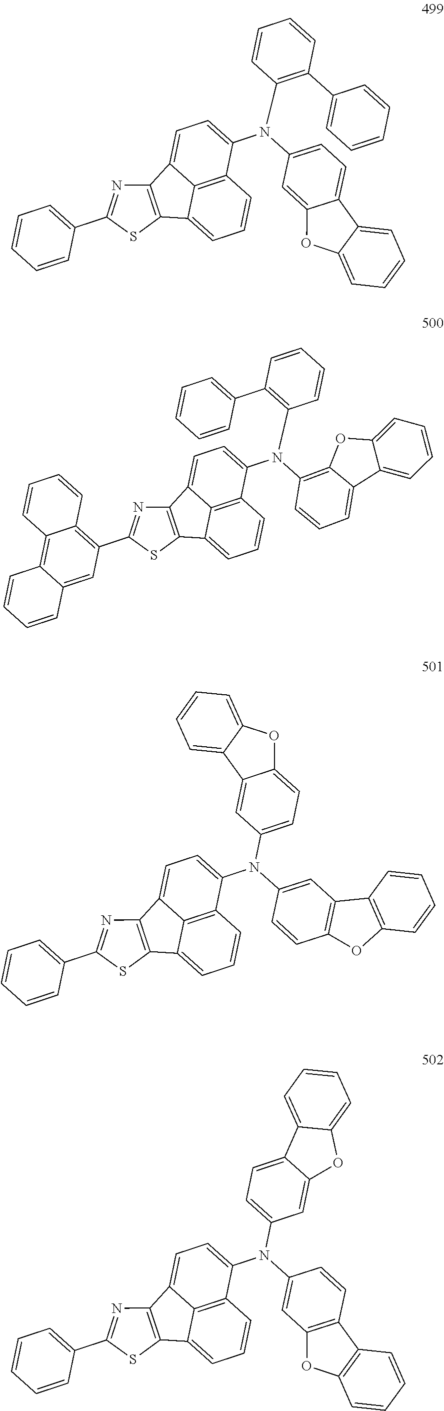 [SMILES: c1ccc(-c2nc3c(s2)-c2cccc4c(N(c5ccc6c(c5)oc5ccccc56)c5ccccc5-c5ccccc5)ccc-3c24)cc1 ; c1ccc(-c2ccccc2N(c2ccc3c4c(cccc24)-c2sc(-c4cc5ccccc5c5ccccc45)nc2-3)c2cccc3c2oc2ccccc23)cc1 ; c1ccc(-c2nc3c(s2)-c2cccc4c(N(c5ccc6oc7ccccc7c6c5)c5ccc6oc7ccccc7c6c5)ccc-3c24)cc1 ; c1ccc(-c2nc3c(s2)-c2cccc4c(N(c5ccc6c(c5)oc5ccccc56)c5ccc6c(c5)oc5ccccc56)ccc-3c24)cc1]

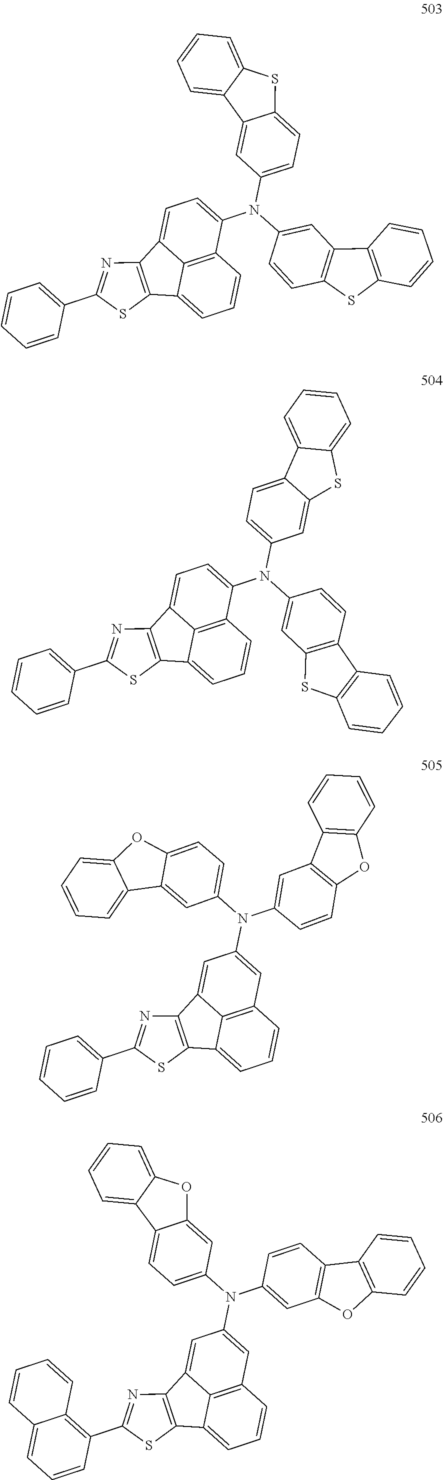 [SMILES: c1ccc(-c2nc3c(s2)-c2cccc4c(N(c5ccc6sc7ccccc7c6c5)c5ccc6sc7ccccc7c6c5)ccc-3c24)cc1 ; c1ccc(-c2nc3c(s2)-c2cccc4c(N(c5ccc6c(c5)sc5ccccc56)c5ccc6c(c5)sc5ccccc56)ccc-3c24)cc1 ; c1ccc(-c2nc3c(s2)-c2cccc4cc(N(c5ccc6oc7ccccc7c6c5)c5ccc6oc7ccccc7c6c5)cc-3c24)cc1 ; c1ccc2c(-c3nc4c(s3)-c3cccc5cc(N(c6ccc7c(c6)oc6ccccc67)c6ccc7c(c6)oc6ccccc67)cc-4c35)cccc2c1]

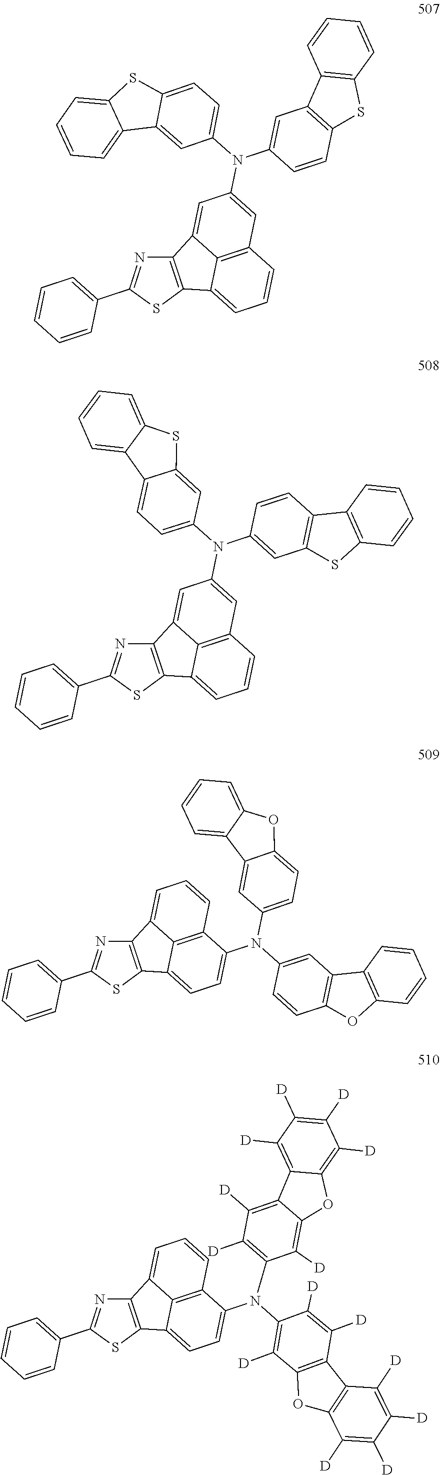 [SMILES: c1ccc(-c2nc3c(s2)-c2cccc4cc(N(c5ccc6sc7ccccc7c6c5)c5ccc6sc7ccccc7c6c5)cc-3c24)cc1 ; c1ccc(-c2nc3c(s2)-c2cccc4cc(N(c5ccc6c(c5)sc5ccccc56)c5ccc6c(c5)sc5ccccc56)cc-3c24)cc1 ; c1ccc(-c2nc3c(s2)-c2ccc(N(c4ccc5oc6ccccc6c5c4)c4ccc5oc6ccccc6c5c4)c4cccc-3c24)cc1 ; [2H]c1c([2H])c([2H])c2c(oc3c([2H])c(N(c4c([2H])c([2H])c5c(oc6c([2H])c([2H])c([2H])c([2H])c65)c4[2H])c4ccc5c6c(cccc46)-c4nc(-c6ccccc6)sc4-5)c([2H])c([2H])c32)c1[2H]]

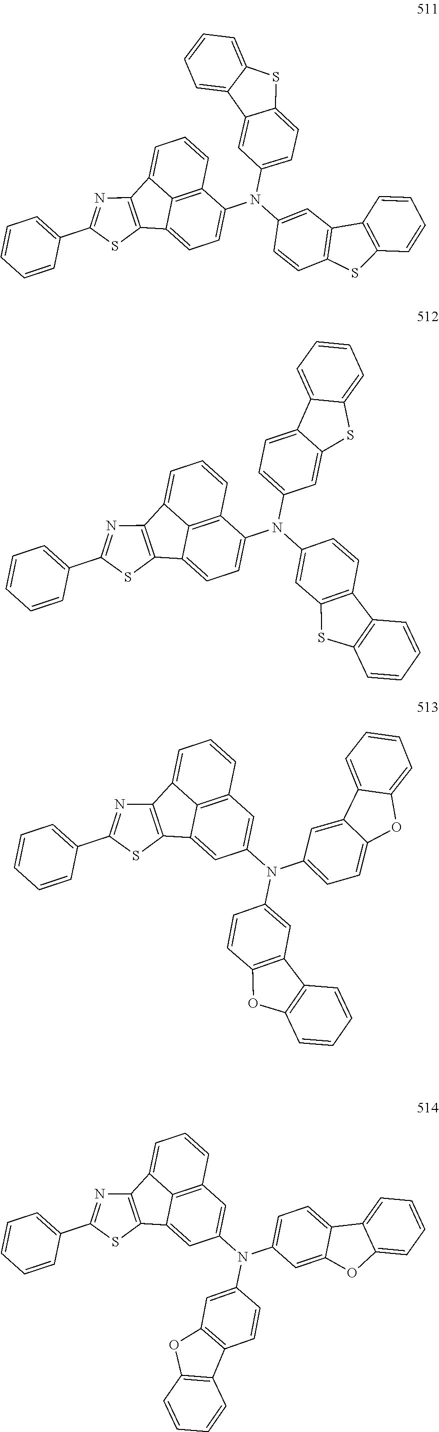 [SMILES: c1ccc(-c2nc3c(s2)-c2ccc(N(c4ccc5sc6ccccc6c5c4)c4ccc5sc6ccccc6c5c4)c4cccc-3c24)cc1 ; c1ccc(-c2nc3c(s2)-c2ccc(N(c4ccc5c(c4)sc4ccccc45)c4ccc5c(c4)sc4ccccc45)c4cccc-3c24)cc1 ; c1ccc(-c2nc3c(s2)-c2cc(N(c4ccc5oc6ccccc6c5c4)c4ccc5oc6ccccc6c5c4)cc4cccc-3c24)cc1 ; c1ccc(-c2nc3c(s2)-c2cc(N(c4ccc5c(c4)oc4ccccc45)c4ccc5c(c4)oc4ccccc45)cc4cccc-3c24)cc1]

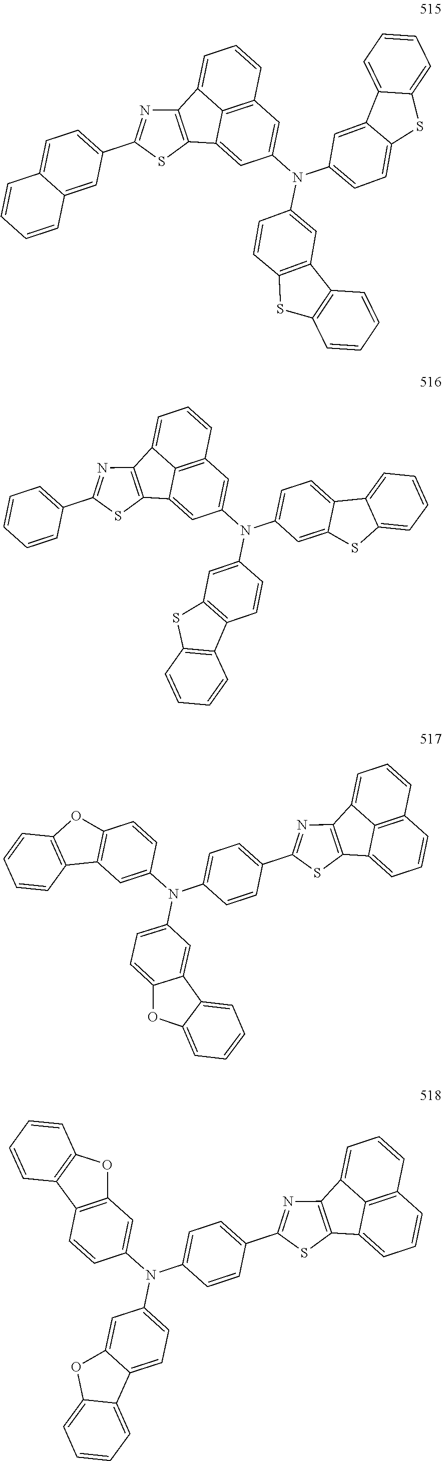 [SMILES: c1ccc2cc(-c3nc4c(s3)-c3cc(N(c5ccc6sc7ccccc7c6c5)c5ccc6sc7ccccc7c6c5)cc5cccc-4c35)ccc2c1 ; c1ccc(-c2nc3c(s2)-c2cc(N(c4ccc5c(c4)sc4ccccc45)c4ccc5c(c4)sc4ccccc45)cc4cccc-3c24)cc1 ; c1cc2c3c(cccc3c1)-c1sc(-c3ccc(N(c4ccc5oc6ccccc6c5c4)c4ccc5oc6ccccc6c5c4)cc3)nc1-2 ; c1cc2c3c(cccc3c1)-c1sc(-c3ccc(N(c4ccc5c(c4)oc4ccccc45)c4ccc5c(c4)oc4ccccc45)cc3)nc1-2]

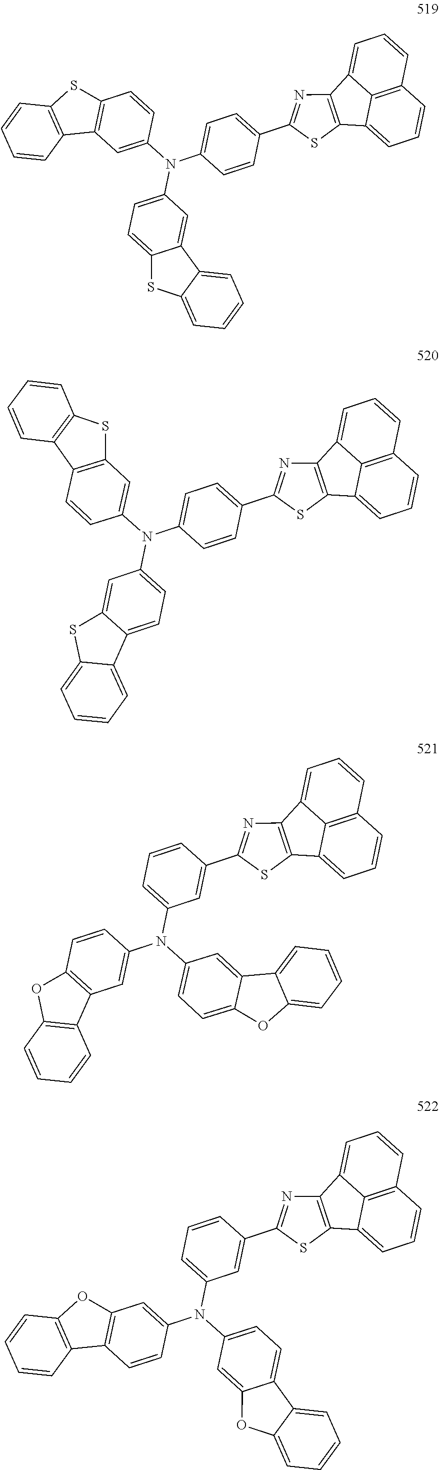 [SMILES: c1cc2c3c(cccc3c1)-c1sc(-c3ccc(N(c4ccc5sc6ccccc6c5c4)c4ccc5sc6ccccc6c5c4)cc3)nc1-2 ; c1cc2c3c(cccc3c1)-c1sc(-c3ccc(N(c4ccc5c(c4)sc4ccccc45)c4ccc5c(c4)sc4ccccc45)cc3)nc1-2 ; c1cc(-c2nc3c(s2)-c2cccc4cccc-3c24)cc(N(c2ccc3oc4ccccc4c3c2)c2ccc3oc4ccccc4c3c2)c1 ; c1cc(-c2nc3c(s2)-c2cccc4cccc-3c24)cc(N(c2ccc3c(c2)oc2ccccc23)c2ccc3c(c2)oc2ccccc23)c1]

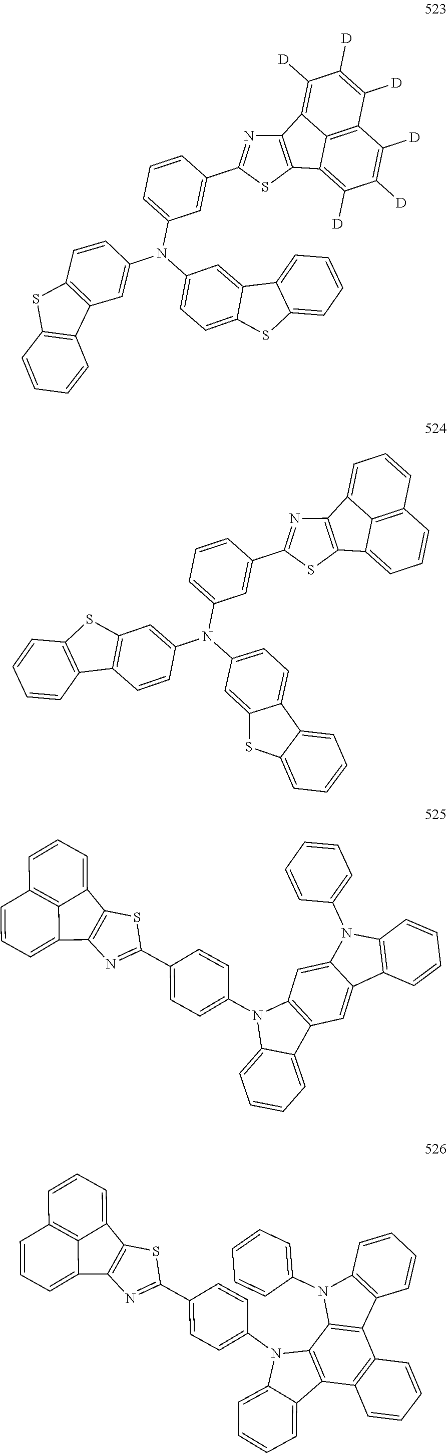 [SMILES: [2H]c1c([2H])c2c3c(c([2H])c([2H])c([2H])c3c1[2H])-c1sc(-c3cccc(N(c4ccc5sc6ccccc6c5c4)c4ccc5sc6ccccc6c5c4)c3)nc1-2 ; c1cc(-c2nc3c(s2)-c2cccc4cccc-3c24)cc(N(c2ccc3c(c2)sc2ccccc23)c2ccc3c(c2)sc2ccccc23)c1 ; c1ccc(-n2c3ccccc3c3cc4c5ccccc5n(-c5ccc(-c6nc7c(s6)-c6cccc8cccc-7c68)cc5)c4cc32)cc1 ; c1ccc(-n2c3ccccc3c3c4ccccc4c4c5ccccc5n(-c5ccc(-c6nc7c(s6)-c6cccc8cccc-7c68)cc5)c4c32)cc1]

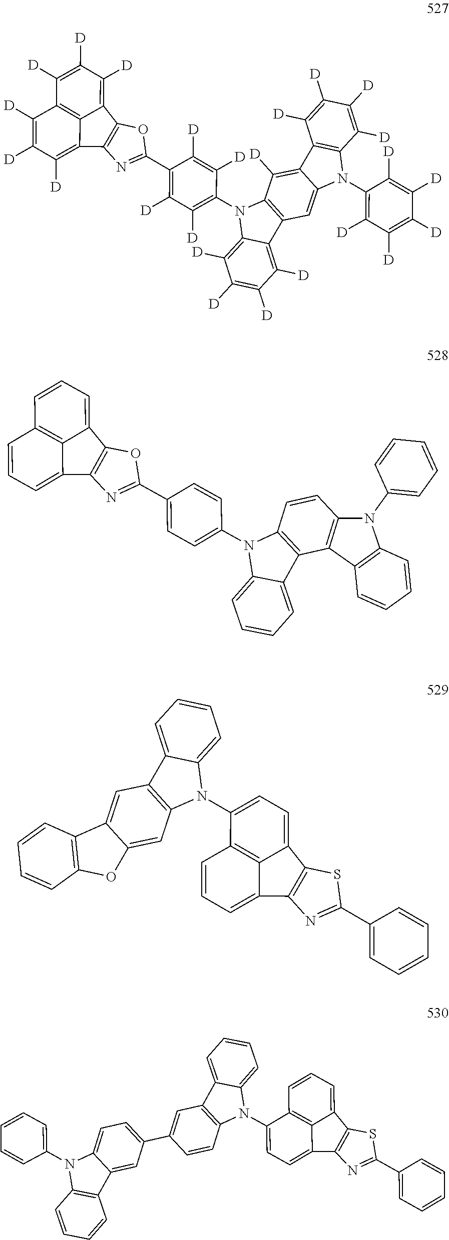 [SMILES: [2H]c1c([2H])c([2H])c(-n2c3cc4c5c([2H])c([2H])c([2H])c([2H])c5n(-c5c([2H])c([2H])c(-c6nc7c(o6)-c6c([2H])c([2H])c([2H])c8c([2H])c([2H])c([2H])c-7c68)c([2H])c5[2H])c4c([2H])c3c3c([2H])c([2H])c([2H])c([2H])c32)c([2H])c1[2H] ; c1ccc(-n2c3ccccc3c3c4c5ccccc5n(-c5ccc(-c6nc7c(o6)-c6cccc8cccc-7c68)cc5)c4ccc32)cc1 ; c1ccc(-c2nc3c(s2)-c2ccc(-n4c5ccccc5c5cc6c(cc54)oc4ccccc46)c4cccc-3c24)cc1 ; c1ccc(-c2nc3c(s2)-c2cccc4c(-n5c6ccccc6c6cc(-c7ccc8c(c7)c7ccccc7n8-c7ccccc7)ccc65)ccc-3c24)cc1]

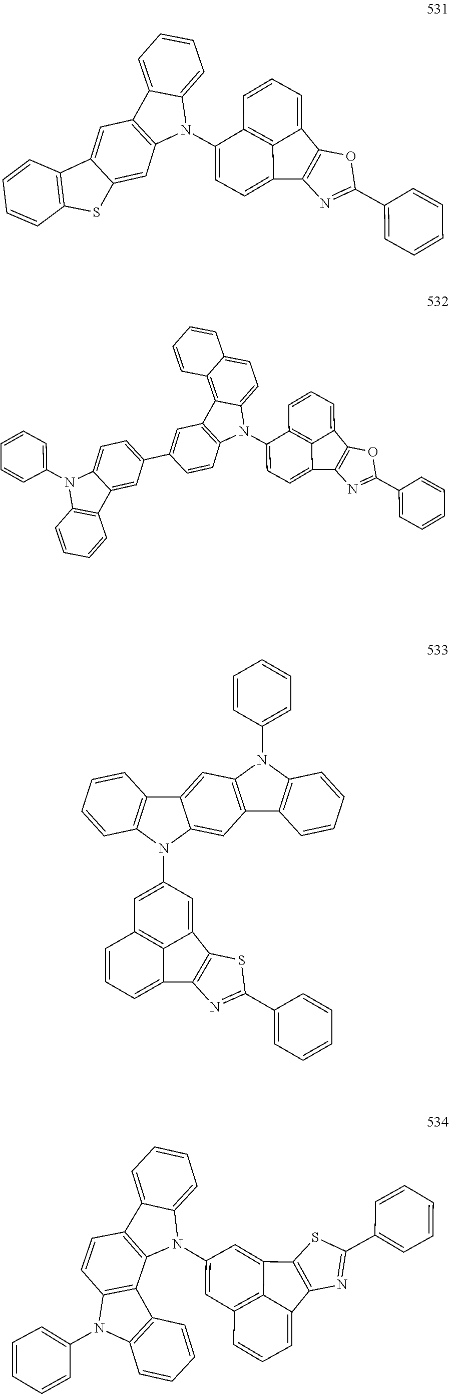 [SMILES: c1ccc(-c2nc3c(o2)-c2cccc4c(-n5c6ccccc6c6cc7c(cc65)sc5ccccc57)ccc-3c24)cc1 ; c1ccc(-c2nc3c(o2)-c2cccc4c(-n5c6ccc(-c7ccc8c(c7)c7ccccc7n8-c7ccccc7)cc6c6c7ccccc7ccc65)ccc-3c24)cc1 ; c1ccc(-c2nc3c(s2)-c2cc(-n4c5ccccc5c5cc6c(cc54)c4ccccc4n6-c4ccccc4)cc4cccc-3c24)cc1 ; c1ccc(-c2nc3c(s2)-c2cc(-n4c5ccccc5c5ccc6c(c7ccccc7n6-c6ccccc6)c54)cc4cccc-3c24)cc1]

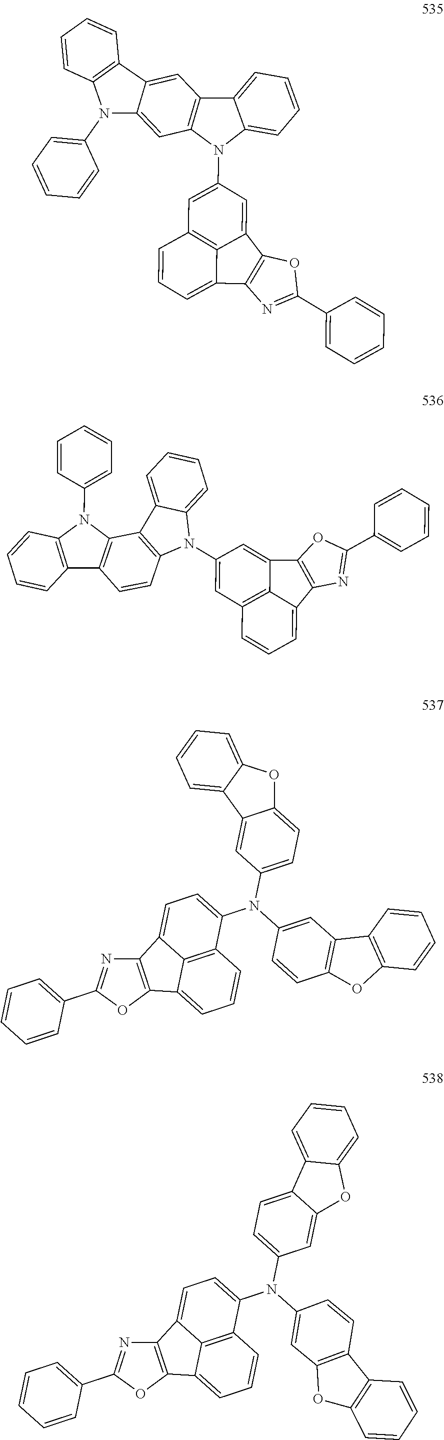 [SMILES: c1ccc(-c2nc3c(o2)-c2cc(-n4c5ccccc5c5cc6c7ccccc7n(-c7ccccc7)c6cc54)cc4cccc-3c24)cc1 ; c1ccc(-c2nc3c(o2)-c2cc(-n4c5ccccc5c5c4ccc4c6ccccc6n(-c6ccccc6)c45)cc4cccc-3c24)cc1 ; c1ccc(-c2nc3c(o2)-c2cccc4c(N(c5ccc6oc7ccccc7c6c5)c5ccc6oc7ccccc7c6c5)ccc-3c24)cc1 ; c1ccc(-c2nc3c(o2)-c2cccc4c(N(c5ccc6c(c5)oc5ccccc56)c5ccc6c(c5)oc5ccccc56)ccc-3c24)cc1]

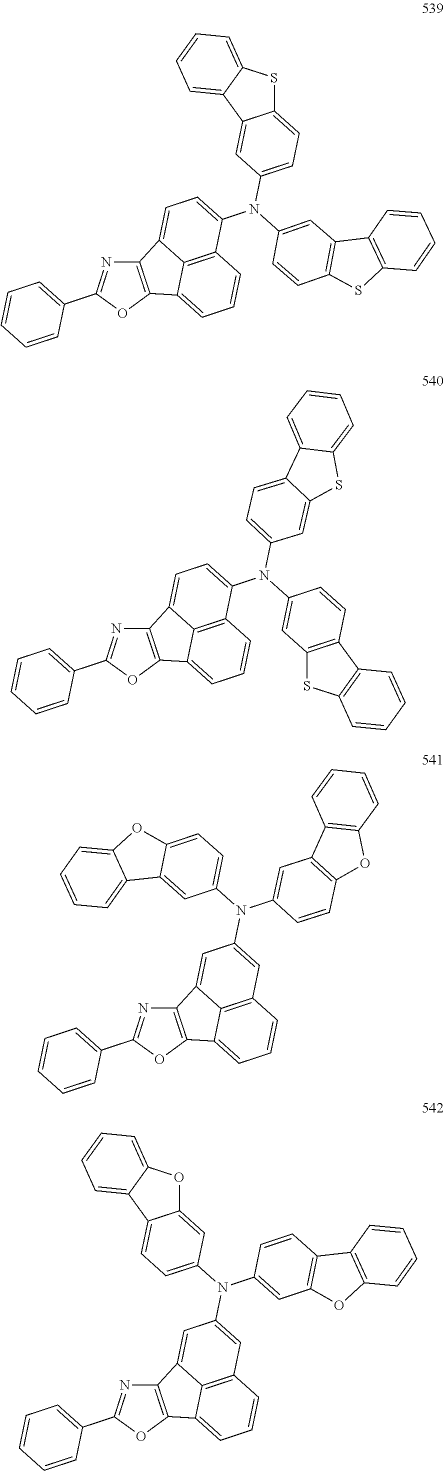 [SMILES: c1ccc(-c2nc3c(o2)-c2cccc4c(N(c5ccc6sc7ccccc7c6c5)c5ccc6sc7ccccc7c6c5)ccc-3c24)cc1 ; c1ccc(-c2nc3c(o2)-c2cccc4c(N(c5ccc6c(c5)sc5ccccc56)c5ccc6c(c5)sc5ccccc56)ccc-3c24)cc1 ; c1ccc(-c2nc3c(o2)-c2cccc4cc(N(c5ccc6oc7ccccc7c6c5)c5ccc6oc7ccccc7c6c5)cc-3c24)cc1 ; c1ccc(-c2nc3c(o2)-c2cccc4cc(N(c5ccc6c(c5)oc5ccccc56)c5ccc6c(c5)oc5ccccc56)cc-3c24)cc1]

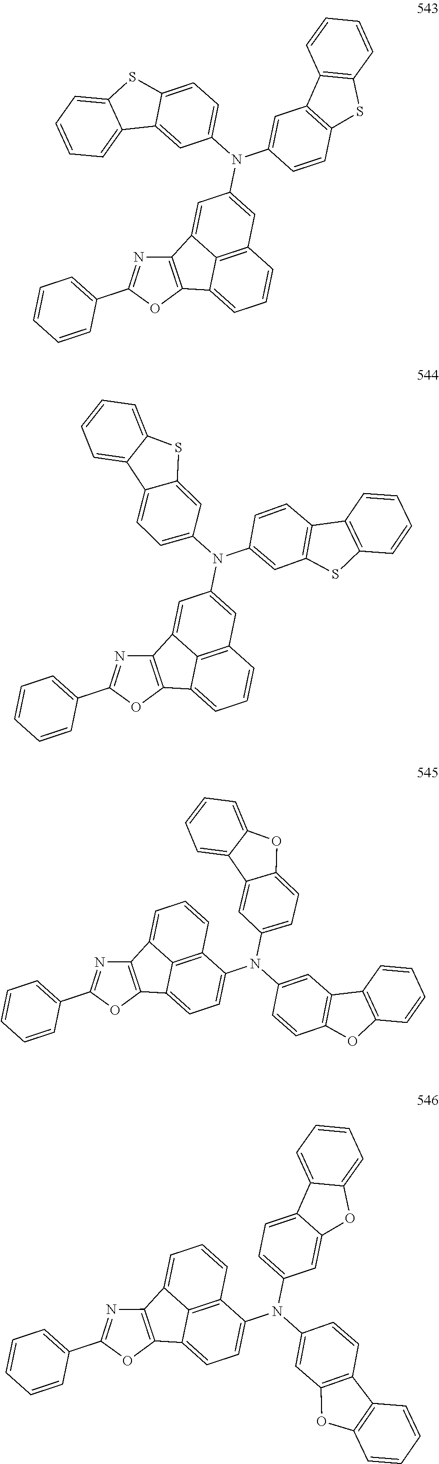 [SMILES: c1ccc(-c2nc3c(o2)-c2cccc4cc(N(c5ccc6sc7ccccc7c6c5)c5ccc6sc7ccccc7c6c5)cc-3c24)cc1 ; c1ccc(-c2nc3c(o2)-c2cccc4cc(N(c5ccc6c(c5)sc5ccccc56)c5ccc6c(c5)sc5ccccc56)cc-3c24)cc1 ; c1ccc(-c2nc3c(o2)-c2ccc(N(c4ccc5oc6ccccc6c5c4)c4ccc5oc6ccccc6c5c4)c4cccc-3c24)cc1 ; c1ccc(-c2nc3c(o2)-c2ccc(N(c4ccc5c(c4)oc4ccccc45)c4ccc5c(c4)oc4ccccc45)c4cccc-3c24)cc1]

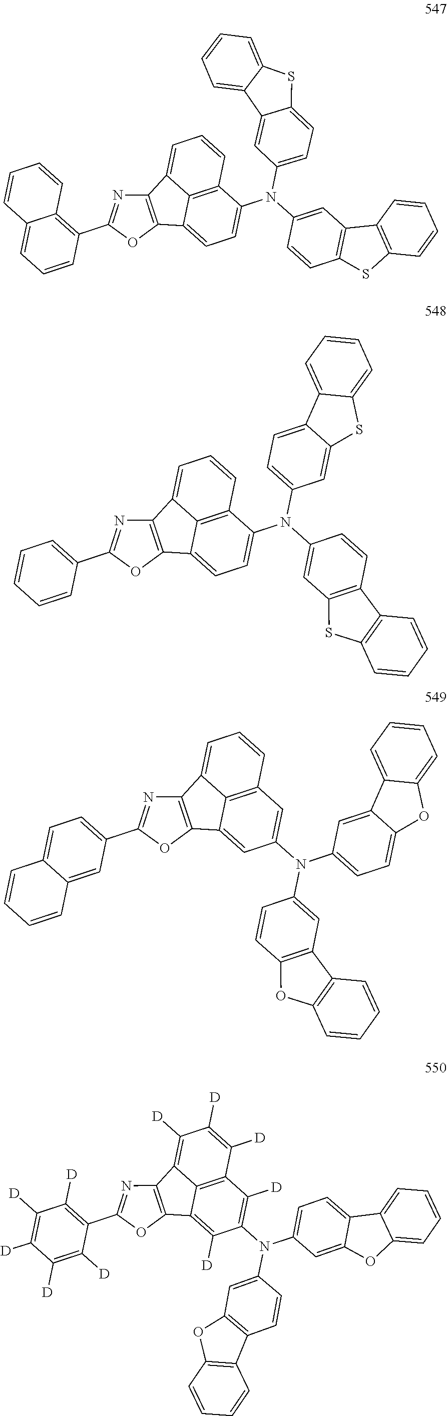 [SMILES: c1ccc2c(-c3nc4c(o3)-c3ccc(N(c5ccc6sc7ccccc7c6c5)c5ccc6sc7ccccc7c6c5)c5cccc-4c35)cccc2c1 ; c1ccc(-c2nc3c(o2)-c2ccc(N(c4ccc5c(c4)sc4ccccc45)c4ccc5c(c4)sc4ccccc45)c4cccc-3c24)cc1 ; c1ccc2cc(-c3nc4c(o3)-c3cc(N(c5ccc6oc7ccccc7c6c5)c5ccc6oc7ccccc7c6c5)cc5cccc-4c35)ccc2c1 ; [2H]c1c([2H])c([2H])c(-c2nc3c(o2)-c2c([2H])c(N(c4ccc5c(c4)oc4ccccc45)c4ccc5c(c4)oc4ccccc45)c([2H])c4c([2H])c([2H])c([2H])c-3c24)c([2H])c1[2H]]

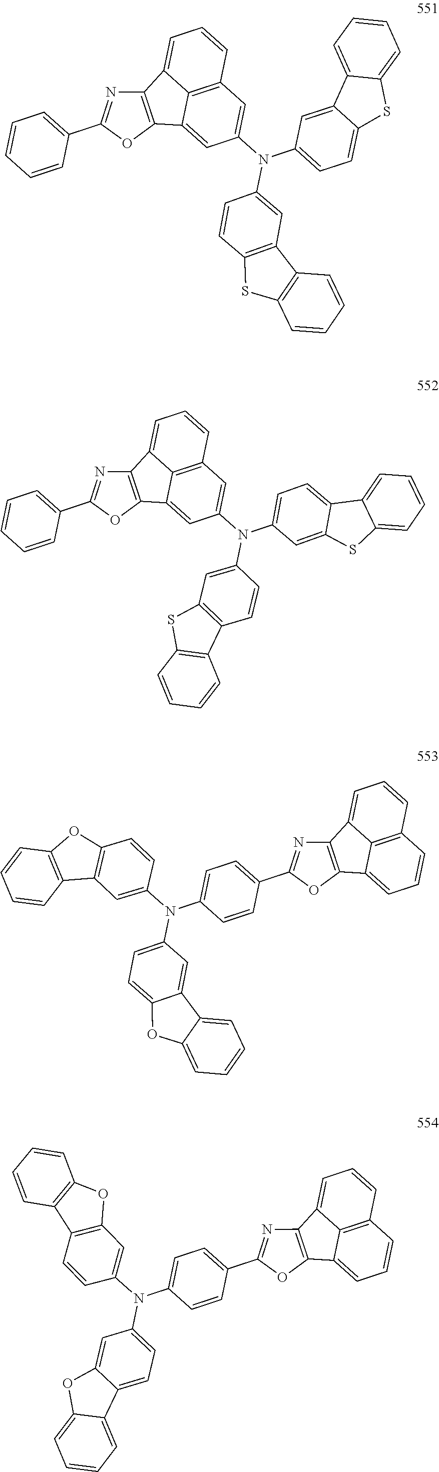 [SMILES: c1ccc(-c2nc3c(o2)-c2cc(N(c4ccc5sc6ccccc6c5c4)c4ccc5sc6ccccc6c5c4)cc4cccc-3c24)cc1 ; c1ccc(-c2nc3c(o2)-c2cc(N(c4ccc5c(c4)sc4ccccc45)c4ccc5c(c4)sc4ccccc45)cc4cccc-3c24)cc1 ; c1cc2c3c(cccc3c1)-c1oc(-c3ccc(N(c4ccc5oc6ccccc6c5c4)c4ccc5oc6ccccc6c5c4)cc3)nc1-2 ; c1cc2c3c(cccc3c1)-c1oc(-c3ccc(N(c4ccc5c(c4)oc4ccccc45)c4ccc5c(c4)oc4ccccc45)cc3)nc1-2]

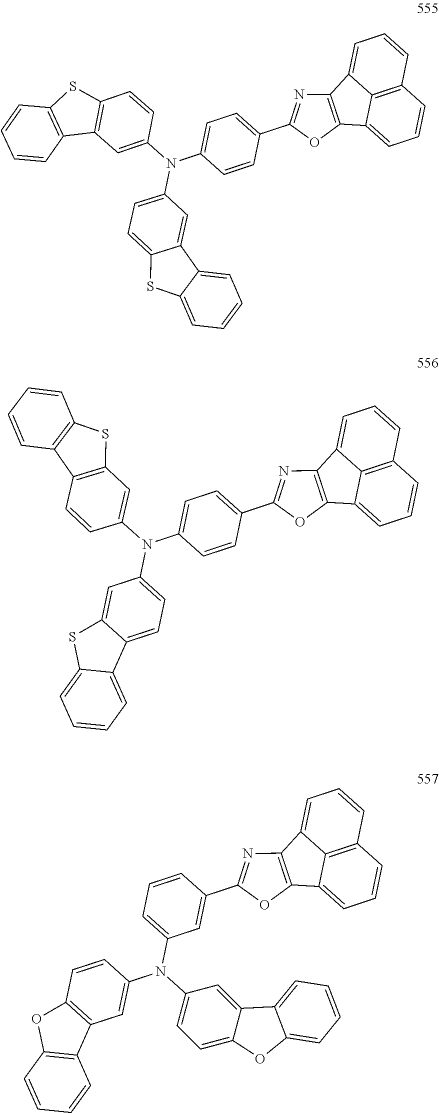 [SMILES: c1cc2c3c(cccc3c1)-c1oc(-c3ccc(N(c4ccc5sc6ccccc6c5c4)c4ccc5sc6ccccc6c5c4)cc3)nc1-2 ; c1cc2c3c(cccc3c1)-c1oc(-c3ccc(N(c4ccc5c(c4)sc4ccccc45)c4ccc5c(c4)sc4ccccc45)cc3)nc1-2 ; c1cc(-c2nc3c(o2)-c2cccc4cccc-3c24)cc(N(c2ccc3oc4ccccc4c3c2)c2ccc3oc4ccccc4c3c2)c1]

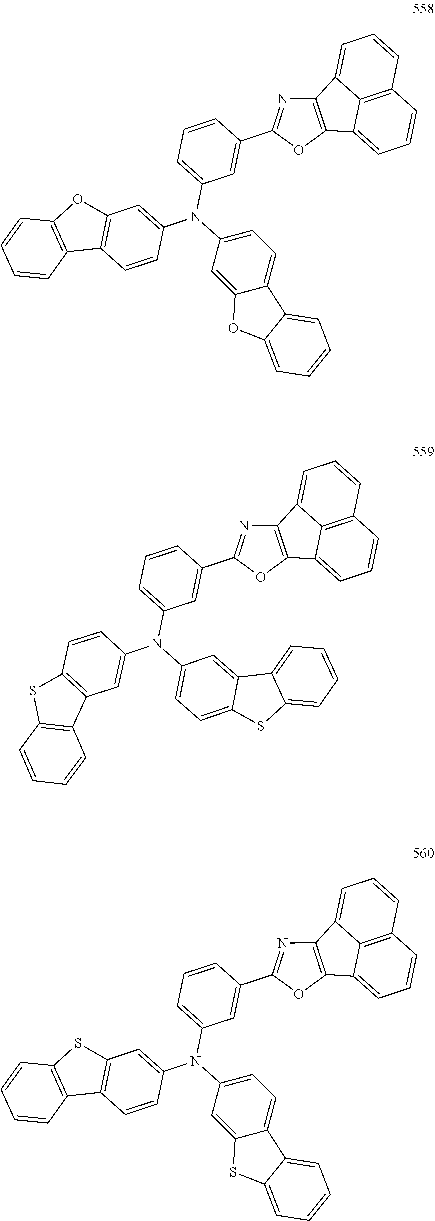 [SMILES: c1cc(-c2nc3c(o2)-c2cccc4cccc-3c24)cc(N(c2ccc3c(c2)oc2ccccc23)c2ccc3c(c2)oc2ccccc23)c1 ; c1cc(-c2nc3c(o2)-c2cccc4cccc-3c24)cc(N(c2ccc3sc4ccccc4c3c2)c2ccc3sc4ccccc4c3c2)c1 ; c1cc(-c2nc3c(o2)-c2cccc4cccc-3c24)cc(N(c2ccc3c(c2)sc2ccccc23)c2ccc3c(c2)sc2ccccc23)c1]

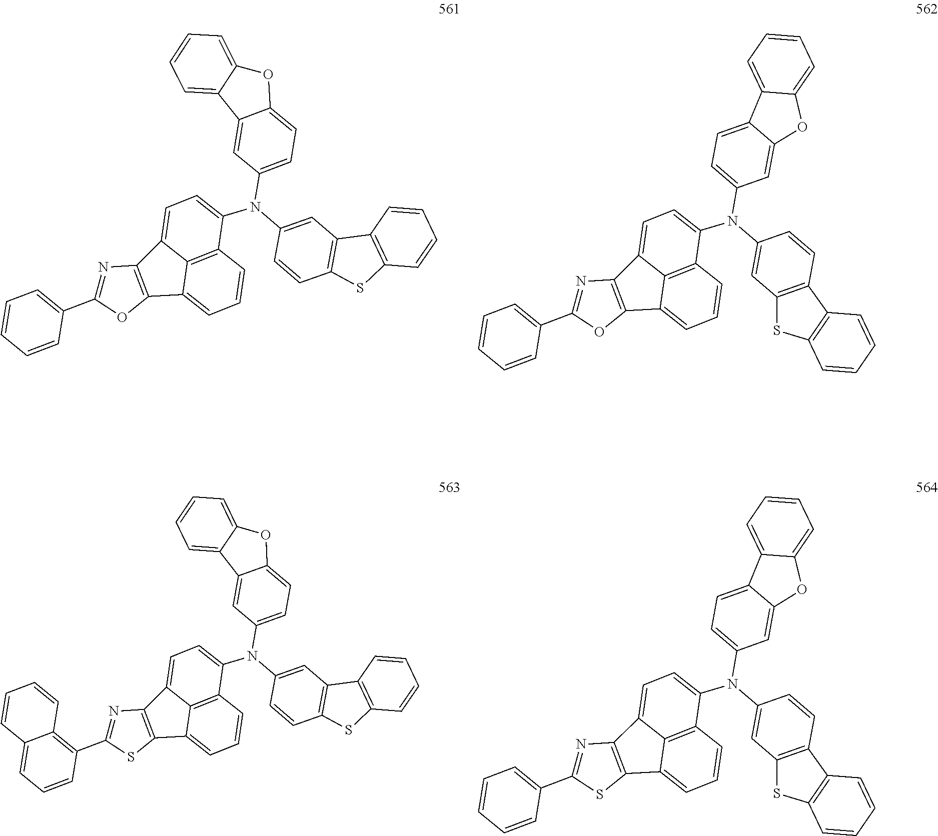 [SMILES: c1ccc(-c2nc3c(o2)-c2cccc4c(N(c5ccc6oc7ccccc7c6c5)c5ccc6sc7ccccc7c6c5)ccc-3c24)cc1 ; c1ccc(-c2nc3c(o2)-c2cccc4c(N(c5ccc6c(c5)oc5ccccc56)c5ccc6c(c5)sc5ccccc56)ccc-3c24)cc1 ; c1ccc2c(-c3nc4c(s3)-c3cccc5c(N(c6ccc7oc8ccccc8c7c6)c6ccc7sc8ccccc8c7c6)ccc-4c35)cccc2c1 ; c1ccc(-c2nc3c(s2)-c2cccc4c(N(c5ccc6c(c5)oc5ccccc56)c5ccc6c(c5)sc5ccccc56)ccc-3c24)cc1]

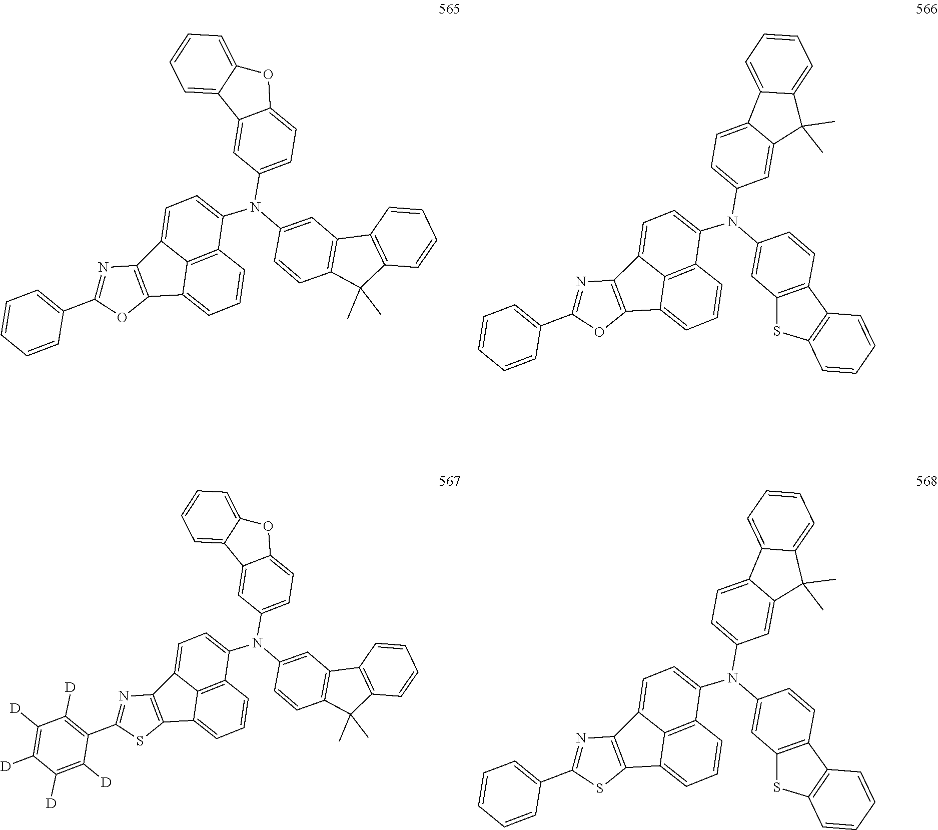 [SMILES: CC1(C)c2ccccc2-c2cc(N(c3ccc4oc5ccccc5c4c3)c3ccc4c5c(cccc35)-c3oc(-c5ccccc5)nc3-4)ccc21 ; CC1(C)c2ccccc2-c2ccc(N(c3ccc4c(c3)sc3ccccc34)c3ccc4c5c(cccc35)-c3oc(-c5ccccc5)nc3-4)cc21 ; [2H]c1c([2H])c([2H])c(-c2nc3c(s2)-c2cccc4c(N(c5ccc6c(c5)-c5ccccc5C6(C)C)c5ccc6oc7ccccc7c6c5)ccc-3c24)c([2H])c1[2H] ; CC1(C)c2ccccc2-c2ccc(N(c3ccc4c(c3)sc3ccccc34)c3ccc4c5c(cccc35)-c3sc(-c5ccccc5)nc3-4)cc21]

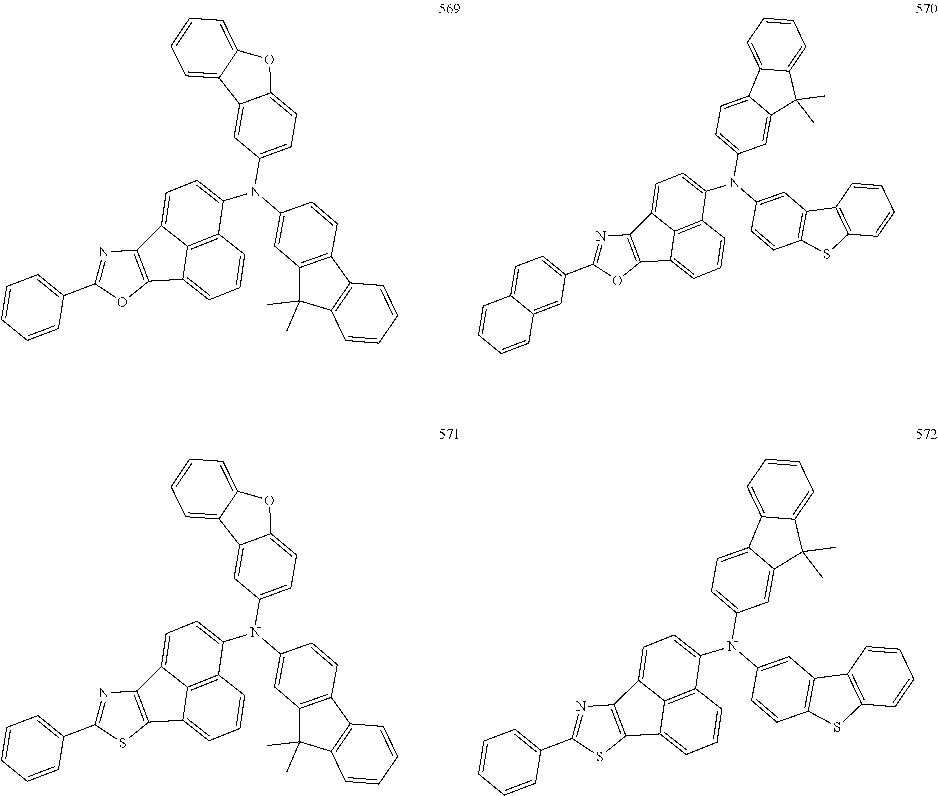 [SMILES: CC1(C)c2ccccc2-c2ccc(N(c3ccc4oc5ccccc5c4c3)c3ccc4c5c(cccc35)-c3oc(-c5ccccc5)nc3-4)cc21 ; CC1(C)c2ccccc2-c2ccc(N(c3ccc4sc5ccccc5c4c3)c3ccc4c5c(cccc35)-c3oc(-c5ccc6ccccc6c5)nc3-4)cc21 ; CC1(C)c2ccccc2-c2ccc(N(c3ccc4oc5ccccc5c4c3)c3ccc4c5c(cccc35)-c3sc(-c5ccccc5)nc3-4)cc21 ; CC1(C)c2ccccc2-c2ccc(N(c3ccc4sc5ccccc5c4c3)c3ccc4c5c(cccc35)-c3sc(-c5ccccc5)nc3-4)cc21]

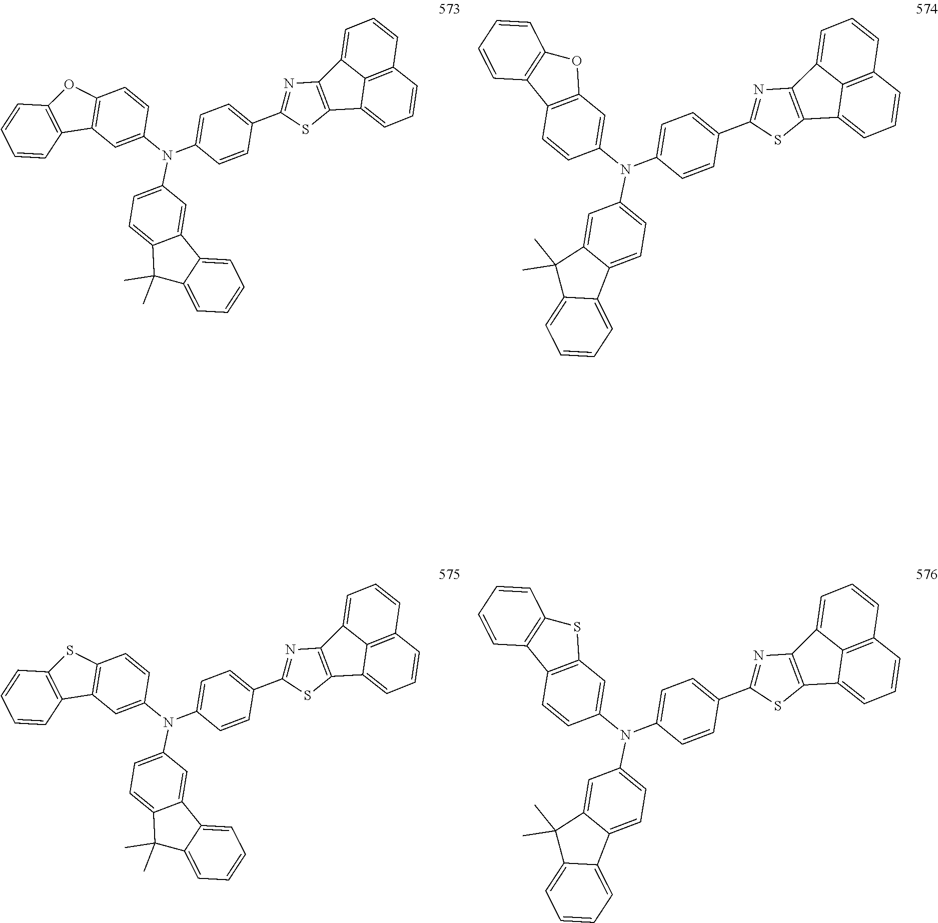 [SMILES: CC1(C)c2ccccc2-c2cc(N(c3ccc(-c4nc5c(s4)-c4cccc6cccc-5c46)cc3)c3ccc4oc5ccccc5c4c3)ccc21 ; CC1(C)c2ccccc2-c2ccc(N(c3ccc(-c4nc5c(s4)-c4cccc6cccc-5c46)cc3)c3ccc4c(c3)oc3ccccc34)cc21 ; CC1(C)c2ccccc2-c2cc(N(c3ccc(-c4nc5c(s4)-c4cccc6cccc-5c46)cc3)c3ccc4sc5ccccc5c4c3)ccc21 ; CC1(C)c2ccccc2-c2ccc(N(c3ccc(-c4nc5c(s4)-c4cccc6cccc-5c46)cc3)c3ccc4c(c3)sc3ccccc34)cc21]

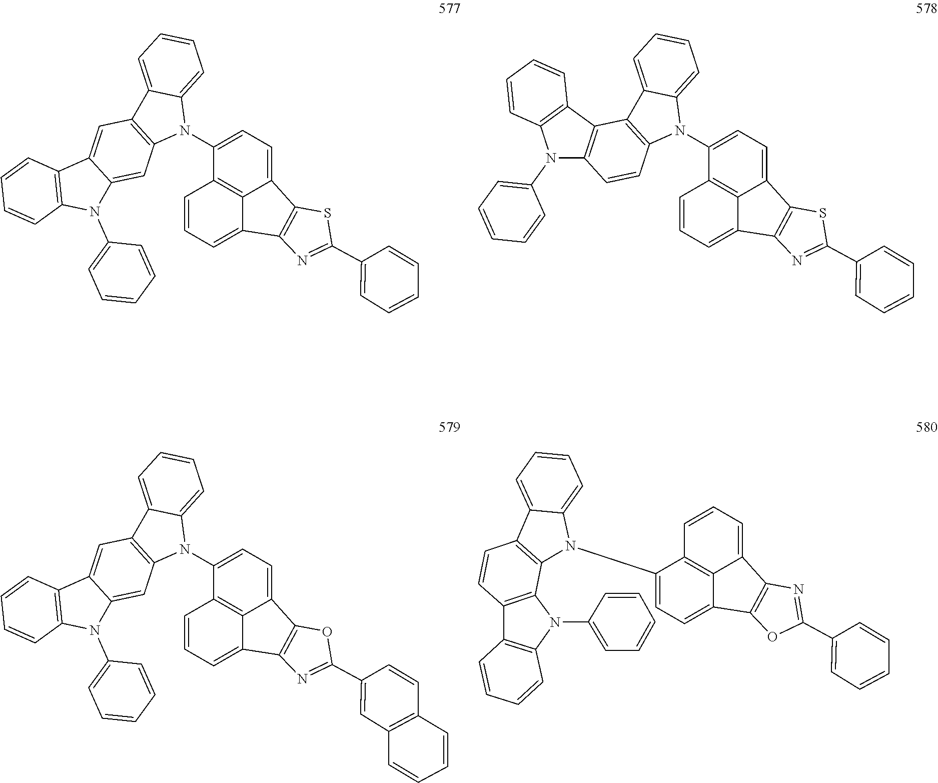 [SMILES: c1ccc(-c2nc3c(s2)-c2ccc(-n4c5ccccc5c5cc6c7ccccc7n(-c7ccccc7)c6cc54)c4cccc-3c24)cc1 ; c1ccc(-c2nc3c(s2)-c2ccc(-n4c5ccccc5c5c6c7ccccc7n(-c7ccccc7)c6ccc54)c4cccc-3c24)cc1 ; c1ccc(-n2c3ccccc3c3cc4c5ccccc5n(-c5ccc6c7c(cccc57)-c5nc(-c7ccc8ccccc8c7)oc5-6)c4cc32)cc1 ; c1ccc(-c2nc3c(o2)-c2ccc(-n4c5ccccc5c5ccc6c7ccccc7n(-c7ccccc7)c6c54)c4cccc-3c24)cc1]

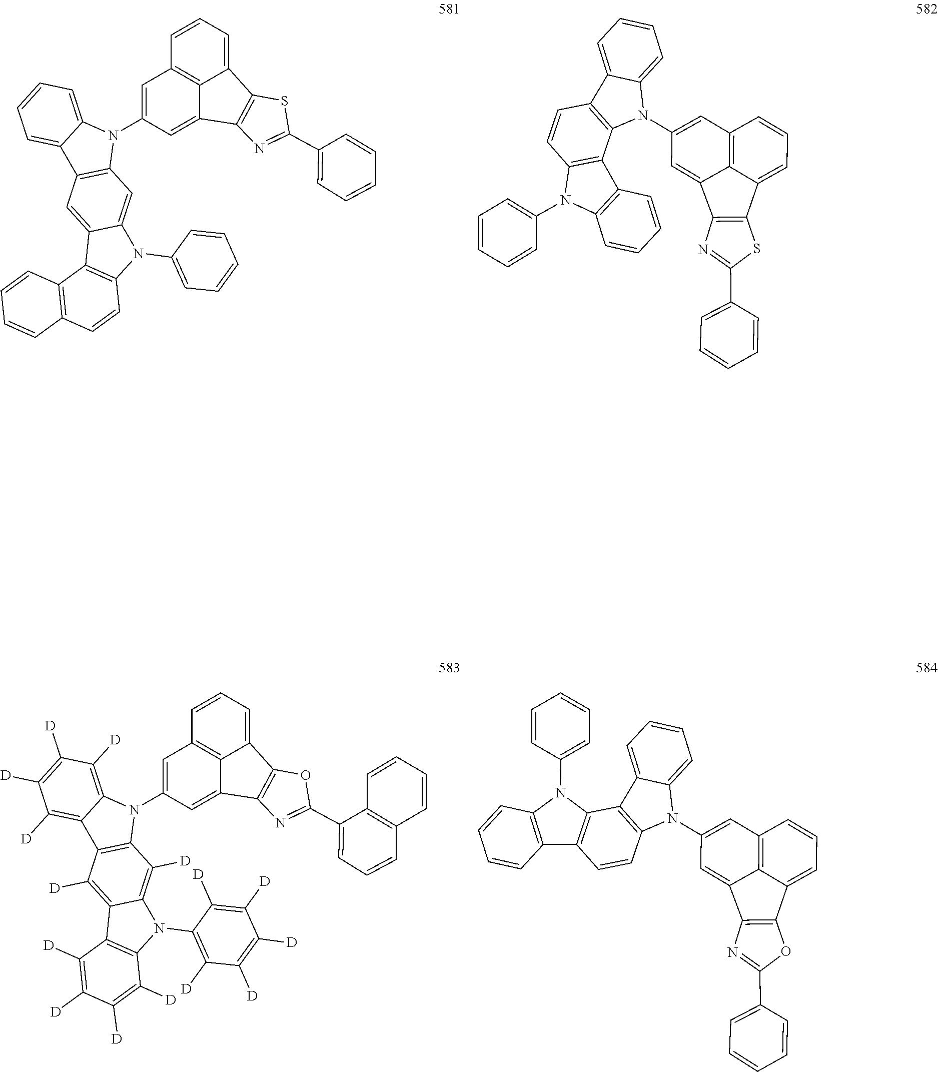 [SMILES: c1ccc(-c2nc3c(s2)-c2cccc4cc(-n5c6ccccc6c6cc7c8c9ccccc9ccc8n(-c8ccccc8)c7cc65)cc-3c24)cc1 ; c1ccc(-c2nc3c(s2)-c2cccc4cc(-n5c6ccccc6c6ccc7c(c8ccccc8n7-c7ccccc7)c65)cc-3c24)cc1 ; [2H]c1c([2H])c([2H])c(-n2c3c([2H])c([2H])c([2H])c([2H])c3c3c([2H])c4c5c([2H])c([2H])c([2H])c([2H])c5n(-c5cc6c7c(cccc7c5)-c5oc(-c7cccc8ccccc78)nc5-6)c4c([2H])c32)c([2H])c1[2H] ; c1ccc(-c2nc3c(o2)-c2cccc4cc(-n5c6ccccc6c6c5ccc5c7ccccc7n(-c7ccccc7)c56)cc-3c24)cc1]

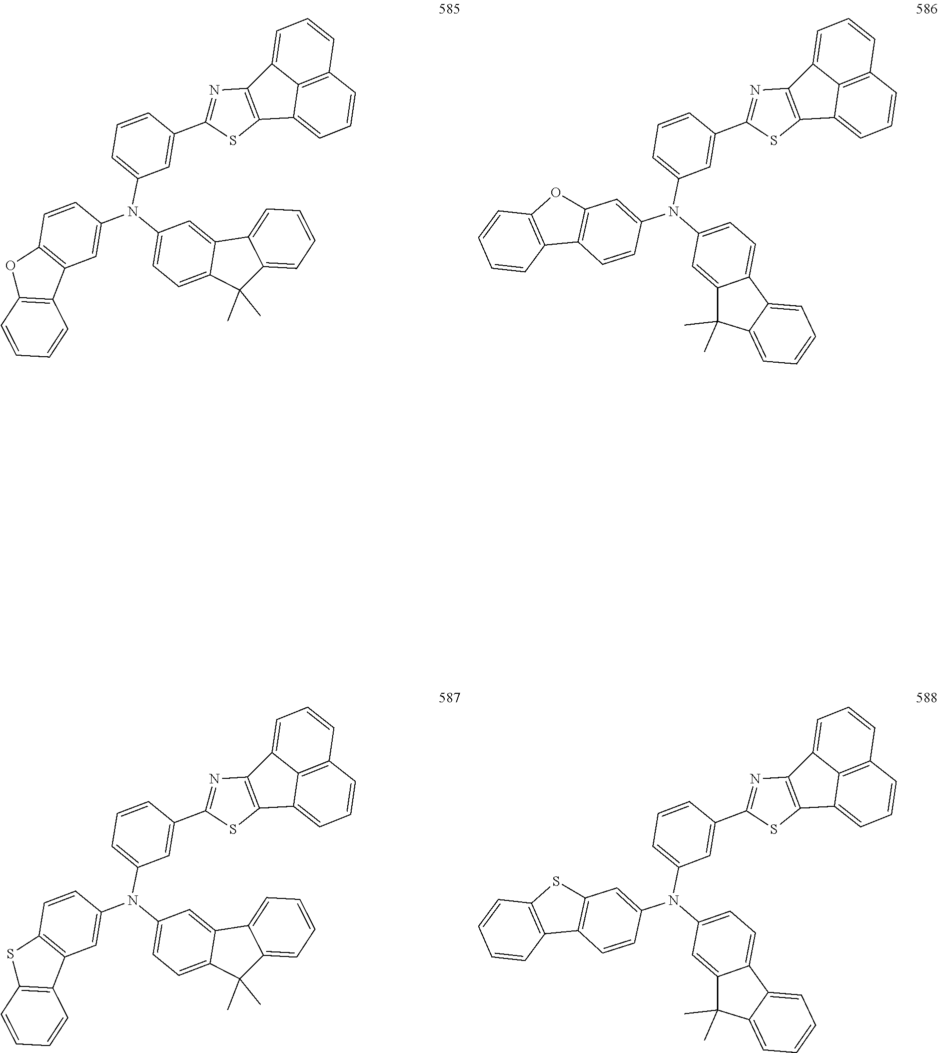 [SMILES: CC1(C)c2ccccc2-c2cc(N(c3cccc(-c4nc5c(s4)-c4cccc6cccc-5c46)c3)c3ccc4oc5ccccc5c4c3)ccc21 ; CC1(C)c2ccccc2-c2ccc(N(c3cccc(-c4nc5c(s4)-c4cccc6cccc-5c46)c3)c3ccc4c(c3)oc3ccccc34)cc21 ; CC1(C)c2ccccc2-c2cc(N(c3cccc(-c4nc5c(s4)-c4cccc6cccc-5c46)c3)c3ccc4sc5ccccc5c4c3)ccc21 ; CC1(C)c2ccccc2-c2ccc(N(c3cccc(-c4nc5c(s4)-c4cccc6cccc-5c46)c3)c3ccc4c(c3)sc3ccccc34)cc21]

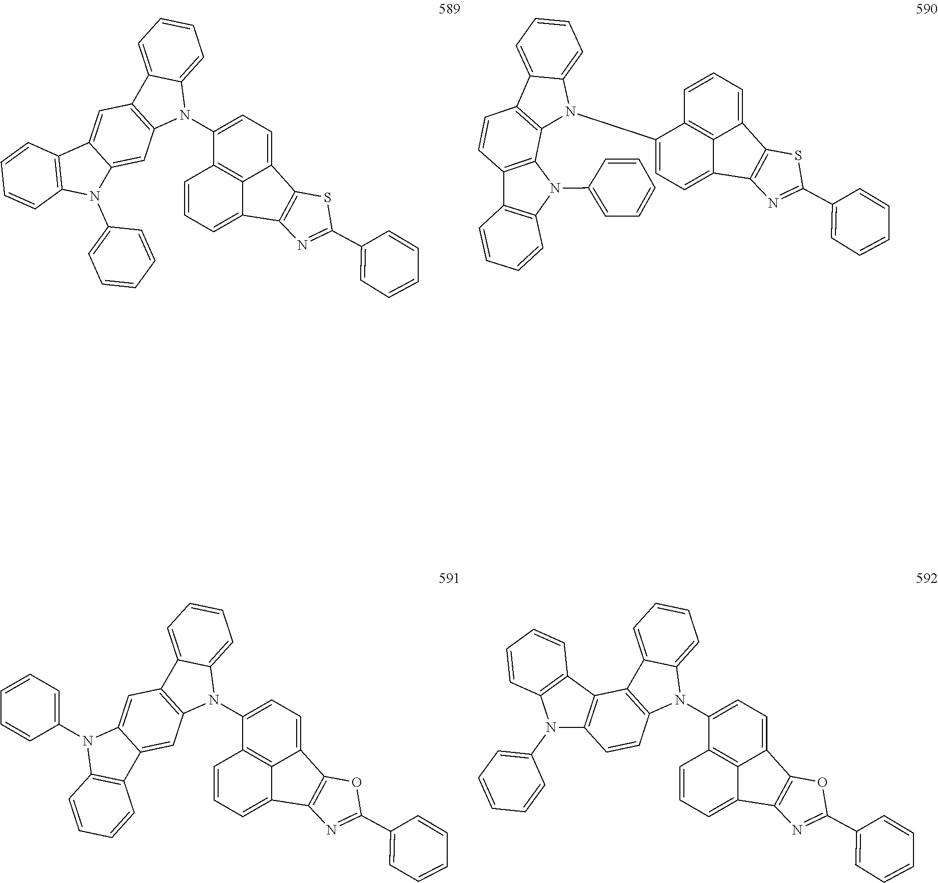 [SMILES: c1ccc(-c2nc3c(s2)-c2ccc(-n4c5ccccc5c5cc6c7ccccc7n(-c7ccccc7)c6cc54)c4cccc-3c24)cc1 ; c1ccc(-c2nc3c(s2)-c2cccc4c(-n5c6ccccc6c6ccc7c8ccccc8n(-c8ccccc8)c7c65)ccc-3c24)cc1 ; c1ccc(-c2nc3c(o2)-c2ccc(-n4c5ccccc5c5cc6c(cc54)c4ccccc4n6-c4ccccc4)c4cccc-3c24)cc1 ; c1ccc(-c2nc3c(o2)-c2ccc(-n4c5ccccc5c5c6c7ccccc7n(-c7ccccc7)c6ccc54)c4cccc-3c24)cc1]

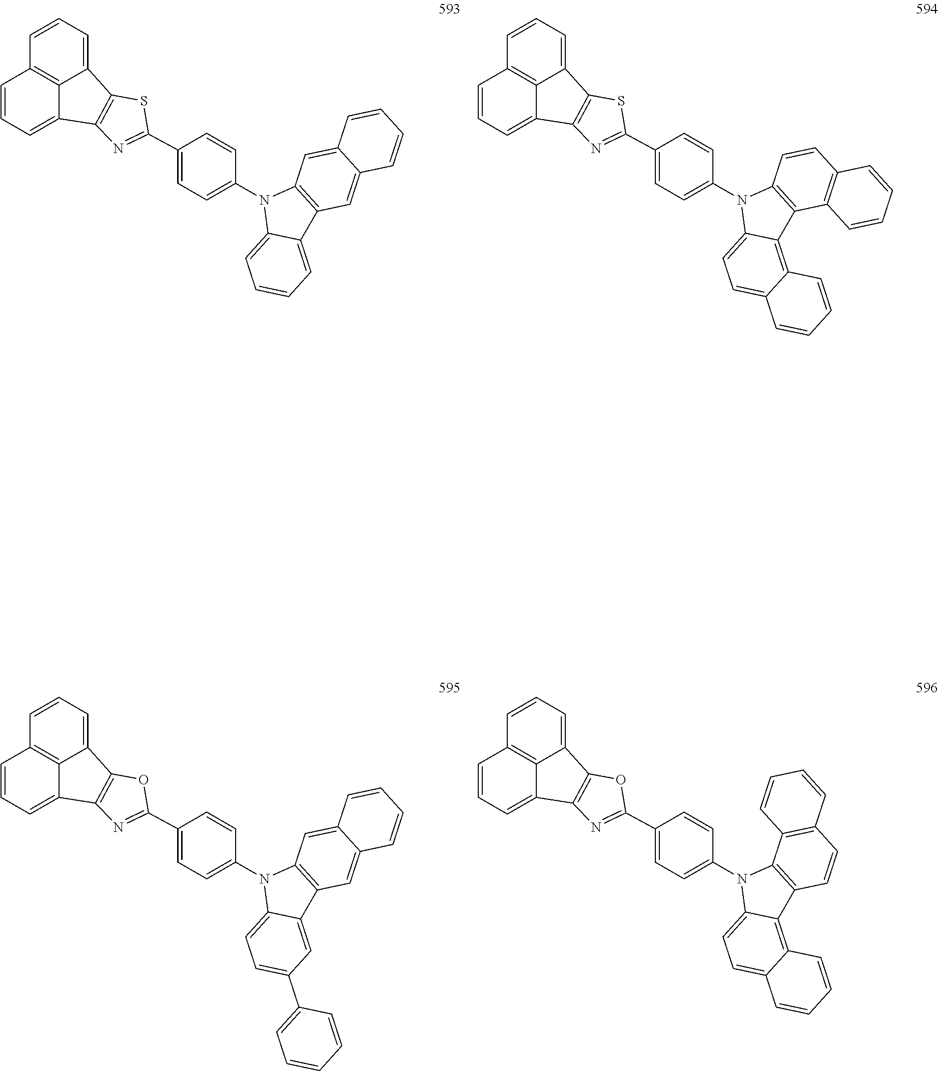 [SMILES: c1ccc2cc3c(cc2c1)c1ccccc1n3-c1ccc(-c2nc3c(s2)-c2cccc4cccc-3c24)cc1 ; c1cc2c3c(cccc3c1)-c1sc(-c3ccc(-n4c5ccc6ccccc6c5c5c6ccccc6ccc54)cc3)nc1-2 ; c1ccc(-c2ccc3c(c2)c2cc4ccccc4cc2n3-c2ccc(-c3nc4c(o3)-c3cccc5cccc-4c35)cc2)cc1 ; c1cc2c3c(cccc3c1)-c1oc(-c3ccc(-n4c5ccc6ccccc6c5c5ccc6ccccc6c54)cc3)nc1-2]

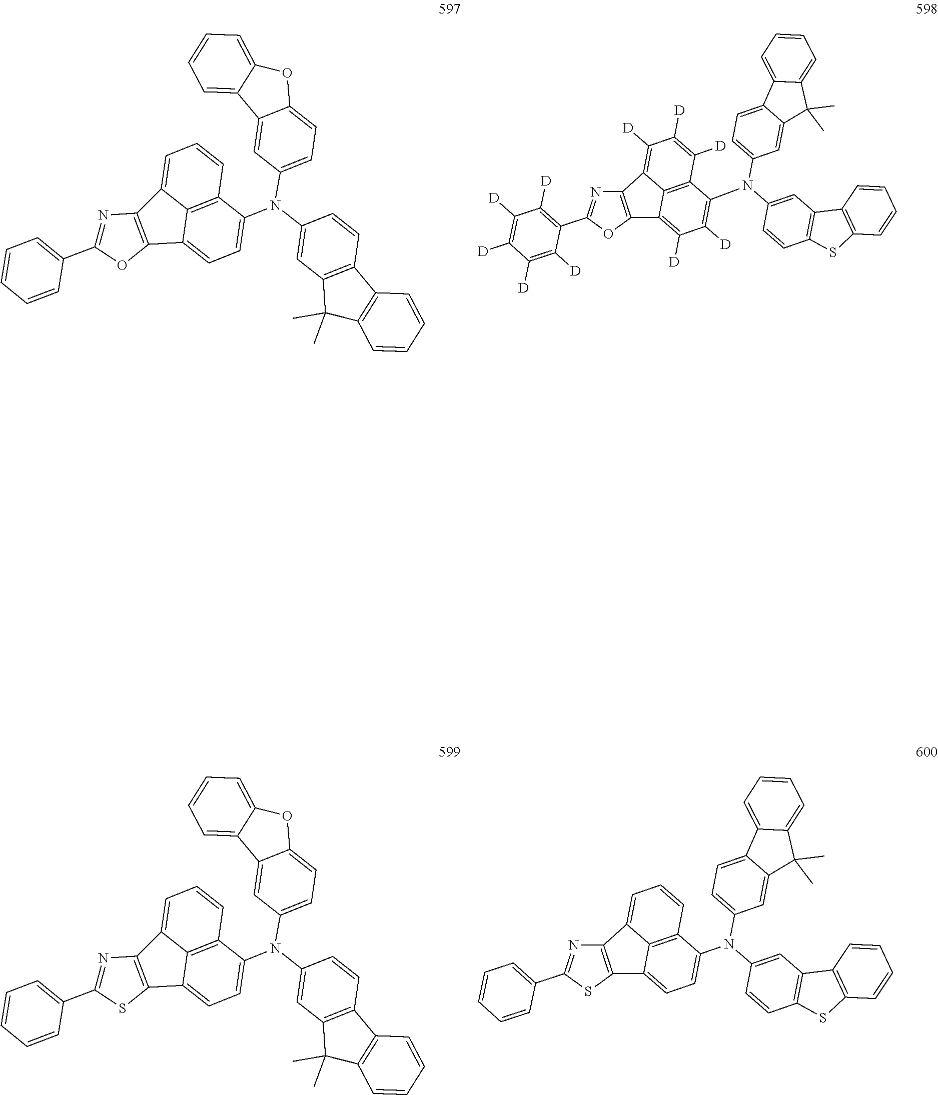 [SMILES: CC1(C)c2ccccc2-c2ccc(N(c3ccc4oc5ccccc5c4c3)c3ccc4c5c(cccc35)-c3nc(-c5ccccc5)oc3-4)cc21 ; [2H]c1c([2H])c([2H])c(-c2nc3c(o2)-c2c([2H])c([2H])c(N(c4ccc5c(c4)C(C)(C)c4ccccc4-5)c4ccc5sc6ccccc6c5c4)c4c([2H])c([2H])c([2H])c-3c24)c([2H])c1[2H] ; CC1(C)c2ccccc2-c2ccc(N(c3ccc4oc5ccccc5c4c3)c3ccc4c5c(cccc35)-c3nc(-c5ccccc5)sc3-4)cc21 ; CC1(C)c2ccccc2-c2ccc(N(c3ccc4sc5ccccc5c4c3)c3ccc4c5c(cccc35)-c3nc(-c5ccccc5)sc3-4)cc21]

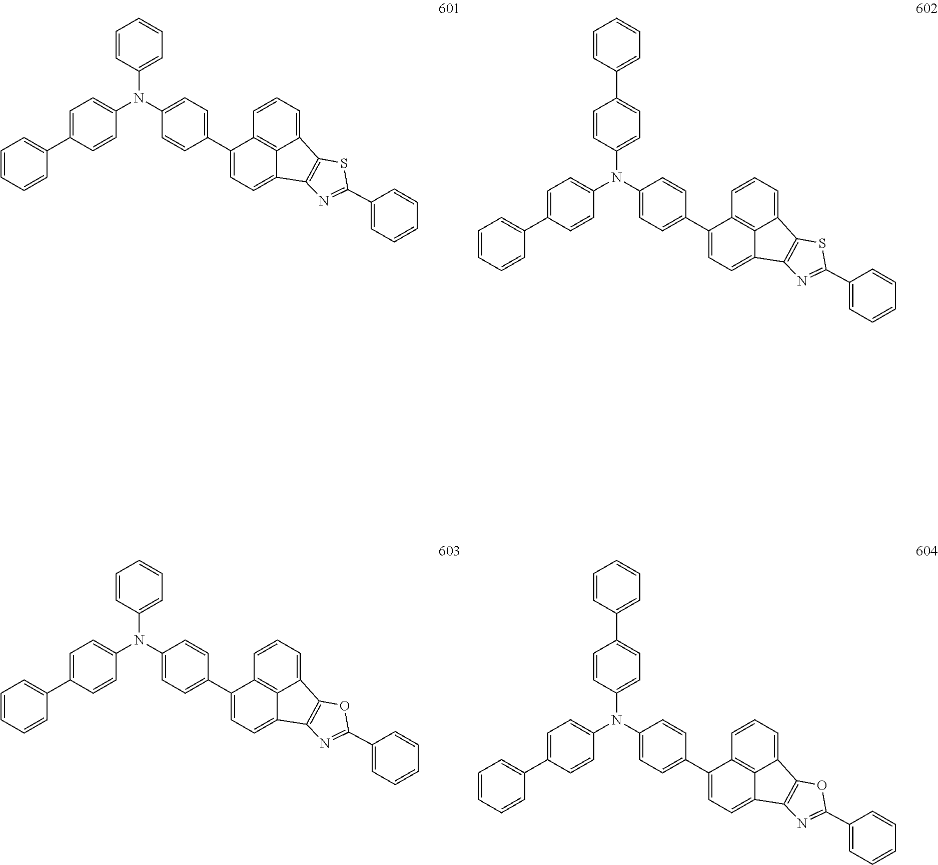 [SMILES: c1ccc(-c2ccc(N(c3ccccc3)c3ccc(-c4ccc5c6c(cccc46)-c4sc(-c6ccccc6)nc4-5)cc3)cc2)cc1 ; c1ccc(-c2ccc(N(c3ccc(-c4ccccc4)cc3)c3ccc(-c4ccc5c6c(cccc46)-c4sc(-c6ccccc6)nc4-5)cc3)cc2)cc1 ; c1ccc(-c2ccc(N(c3ccccc3)c3ccc(-c4ccc5c6c(cccc46)-c4oc(-c6ccccc6)nc4-5)cc3)cc2)cc1 ; c1ccc(-c2ccc(N(c3ccc(-c4ccccc4)cc3)c3ccc(-c4ccc5c6c(cccc46)-c4oc(-c6ccccc6)nc4-5)cc3)cc2)cc1]

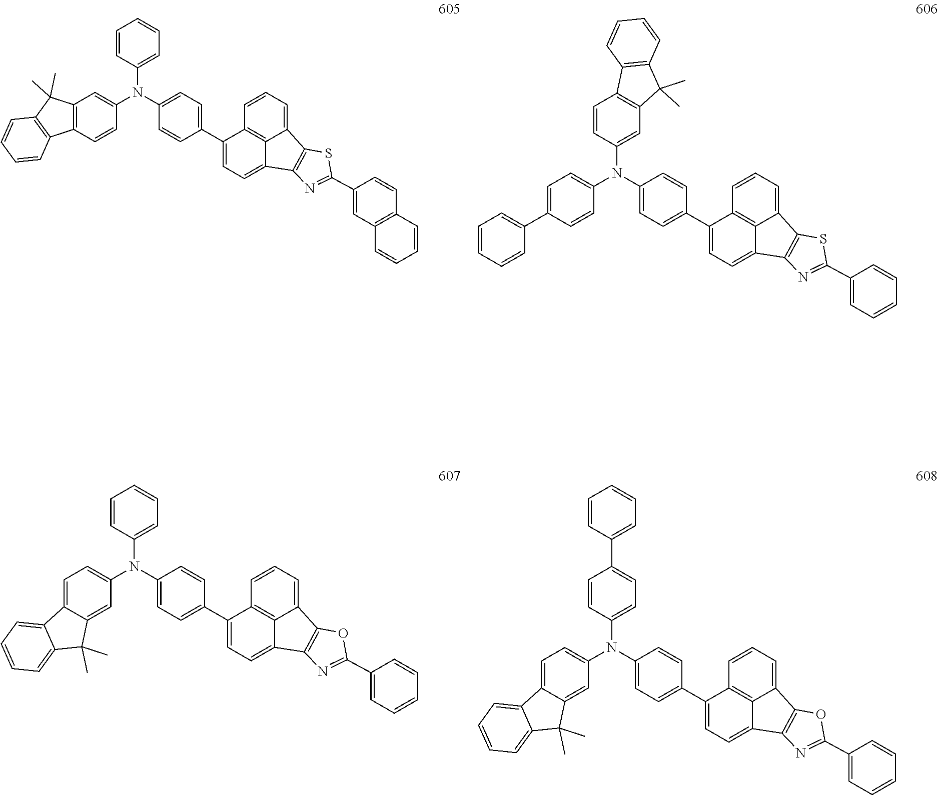 [SMILES: CC1(C)c2ccccc2-c2ccc(N(c3ccccc3)c3ccc(-c4ccc5c6c(cccc46)-c4sc(-c6ccc7ccccc7c6)nc4-5)cc3)cc21 ; CC1(C)c2ccccc2-c2ccc(N(c3ccc(-c4ccccc4)cc3)c3ccc(-c4ccc5c6c(cccc46)-c4sc(-c6ccccc6)nc4-5)cc3)cc21 ; CC1(C)c2ccccc2-c2ccc(N(c3ccccc3)c3ccc(-c4ccc5c6c(cccc46)-c4oc(-c6ccccc6)nc4-5)cc3)cc21 ; CC1(C)c2ccccc2-c2ccc(N(c3ccc(-c4ccccc4)cc3)c3ccc(-c4ccc5c6c(cccc46)-c4oc(-c6ccccc6)nc4-5)cc3)cc21]

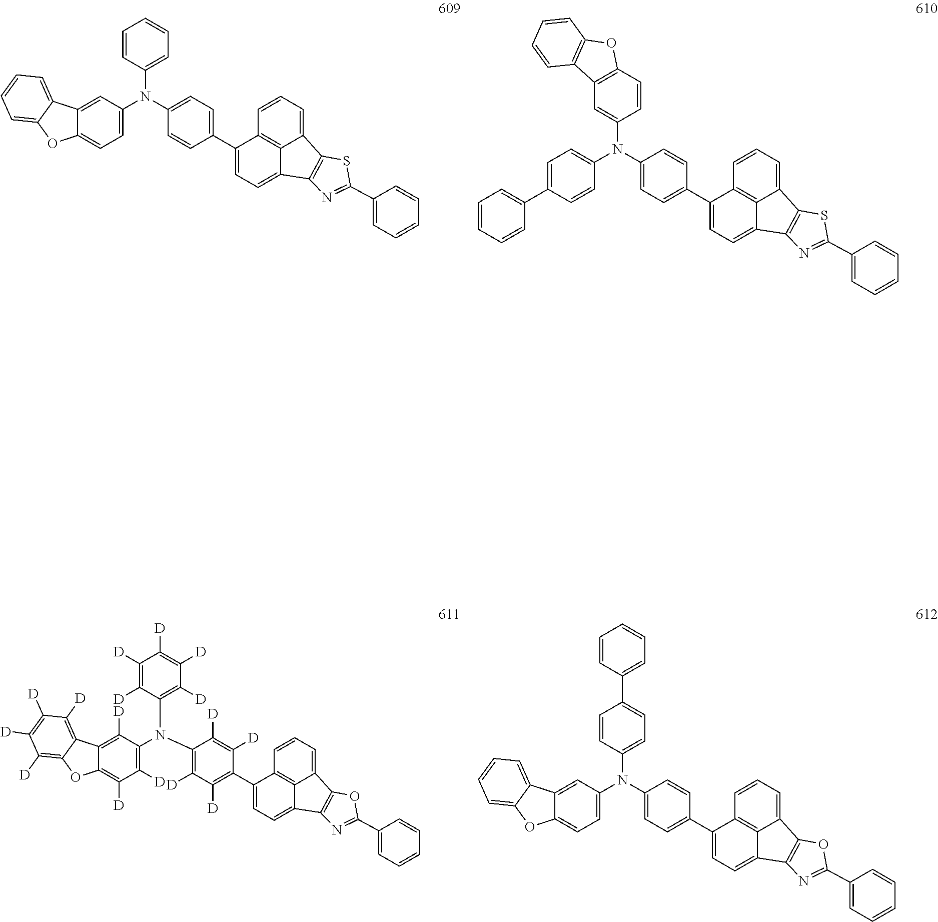 [SMILES: c1ccc(-c2nc3c(s2)-c2cccc4c(-c5ccc(N(c6ccccc6)c6ccc7oc8ccccc8c7c6)cc5)ccc-3c24)cc1 ; c1ccc(-c2ccc(N(c3ccc(-c4ccc5c6c(cccc46)-c4sc(-c6ccccc6)nc4-5)cc3)c3ccc4oc5ccccc5c4c3)cc2)cc1 ; [2H]c1c([2H])c([2H])c(N(c2c([2H])c([2H])c(-c3ccc4c5c(cccc35)-c3oc(-c5ccccc5)nc3-4)c([2H])c2[2H])c2c([2H])c([2H])c3oc4c([2H])c([2H])c([2H])c([2H])c4c3c2[2H])c([2H])c1[2H] ; c1ccc(-c2ccc(N(c3ccc(-c4ccc5c6c(cccc46)-c4oc(-c6ccccc6)nc4-5)cc3)c3ccc4oc5ccccc5c4c3)cc2)cc1]

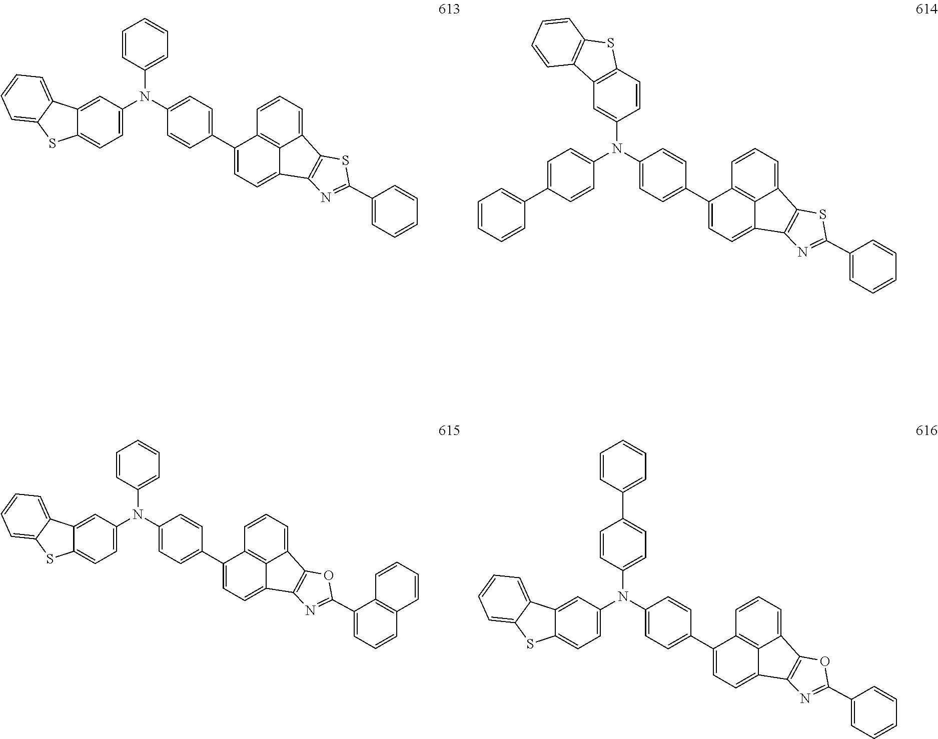 [SMILES: c1ccc(-c2nc3c(s2)-c2cccc4c(-c5ccc(N(c6ccccc6)c6ccc7sc8ccccc8c7c6)cc5)ccc-3c24)cc1 ; c1ccc(-c2ccc(N(c3ccc(-c4ccc5c6c(cccc46)-c4sc(-c6ccccc6)nc4-5)cc3)c3ccc4sc5ccccc5c4c3)cc2)cc1 ; c1ccc(N(c2ccc(-c3ccc4c5c(cccc35)-c3oc(-c5cccc6ccccc56)nc3-4)cc2)c2ccc3sc4ccccc4c3c2)cc1 ; c1ccc(-c2ccc(N(c3ccc(-c4ccc5c6c(cccc46)-c4oc(-c6ccccc6)nc4-5)cc3)c3ccc4sc5ccccc5c4c3)cc2)cc1]

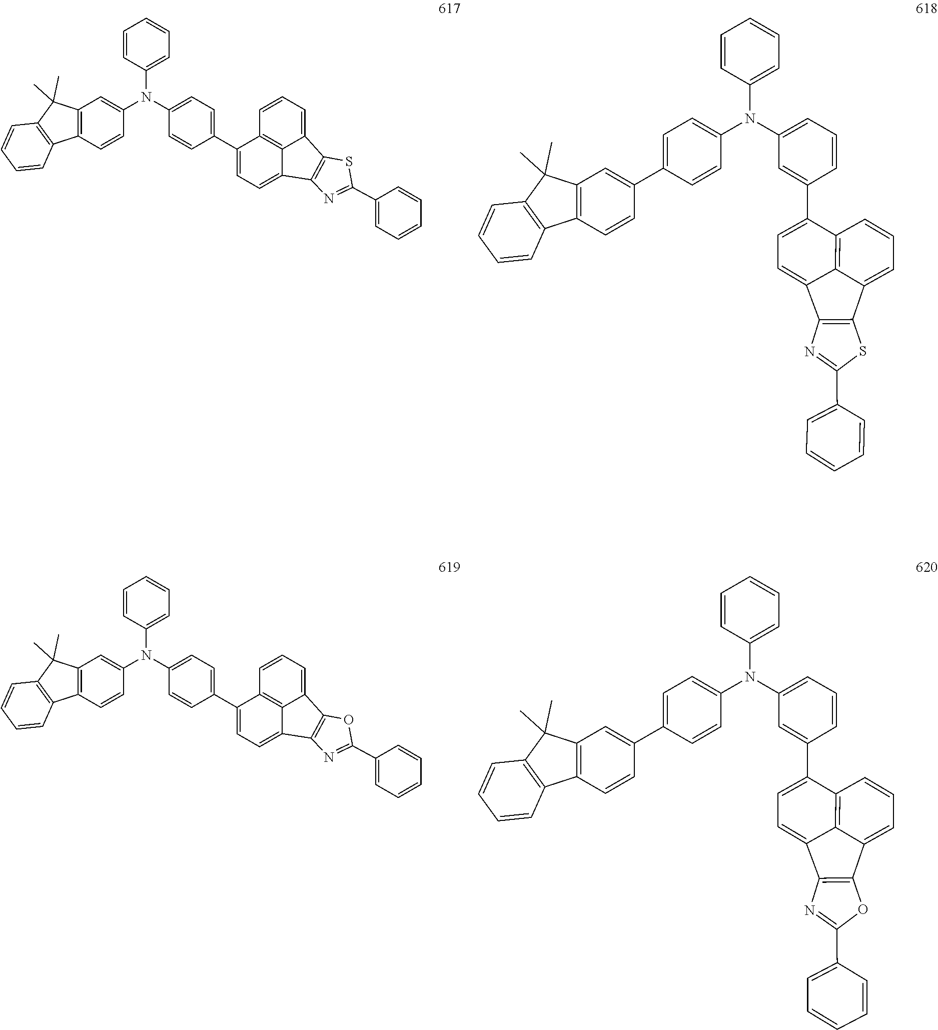 [SMILES: CC1(C)c2ccccc2-c2ccc(N(c3ccccc3)c3ccc(-c4ccc5c6c(cccc46)-c4sc(-c6ccccc6)nc4-5)cc3)cc21 ; CC1(C)c2ccccc2-c2ccc(-c3ccc(N(c4ccccc4)c4cccc(-c5ccc6c7c(cccc57)-c5sc(-c7ccccc7)nc5-6)c4)cc3)cc21 ; CC1(C)c2ccccc2-c2ccc(N(c3ccccc3)c3ccc(-c4ccc5c6c(cccc46)-c4oc(-c6ccccc6)nc4-5)cc3)cc21 ; CC1(C)c2ccccc2-c2ccc(-c3ccc(N(c4ccccc4)c4cccc(-c5ccc6c7c(cccc57)-c5oc(-c7ccccc7)nc5-6)c4)cc3)cc21]

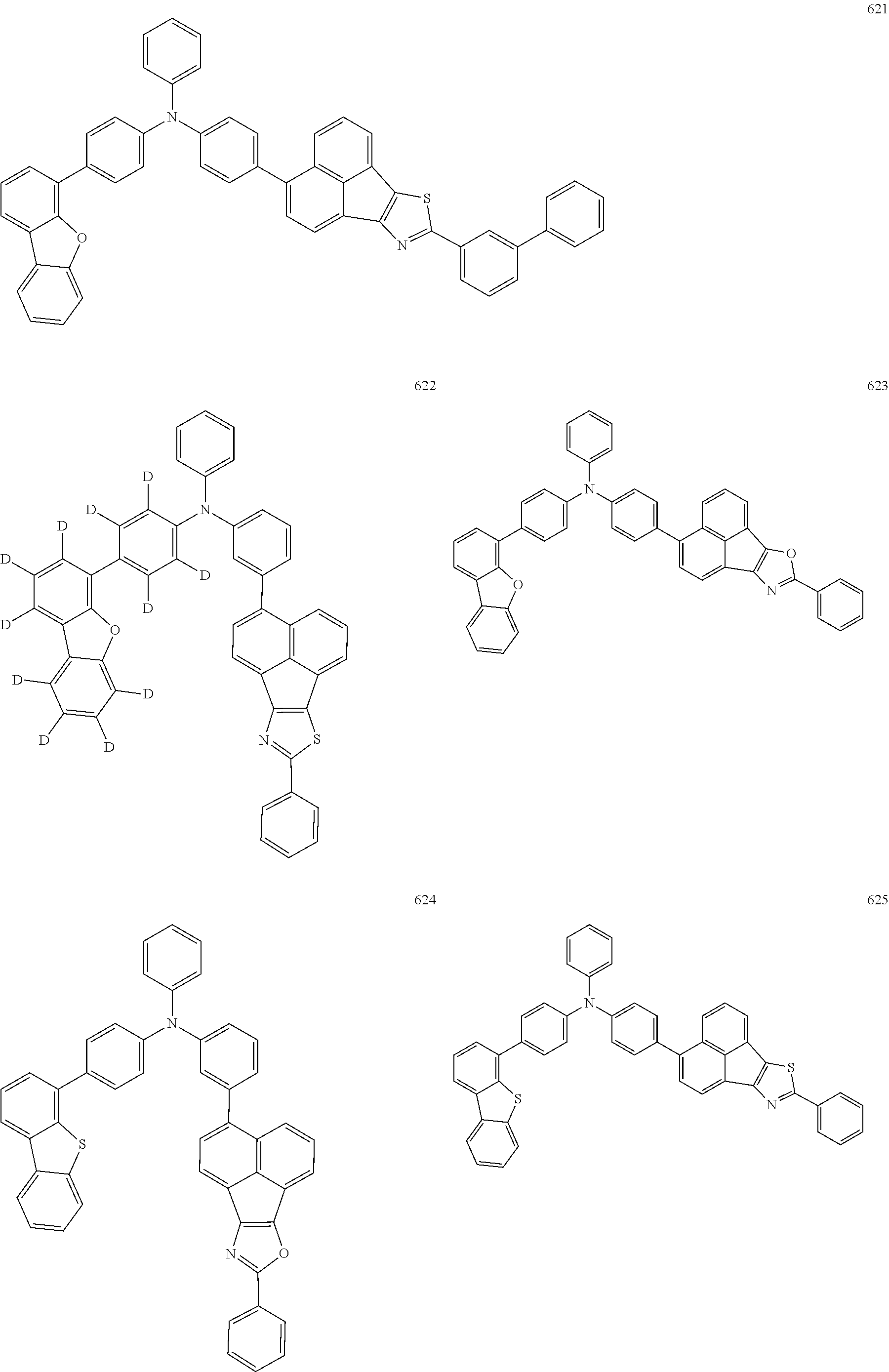

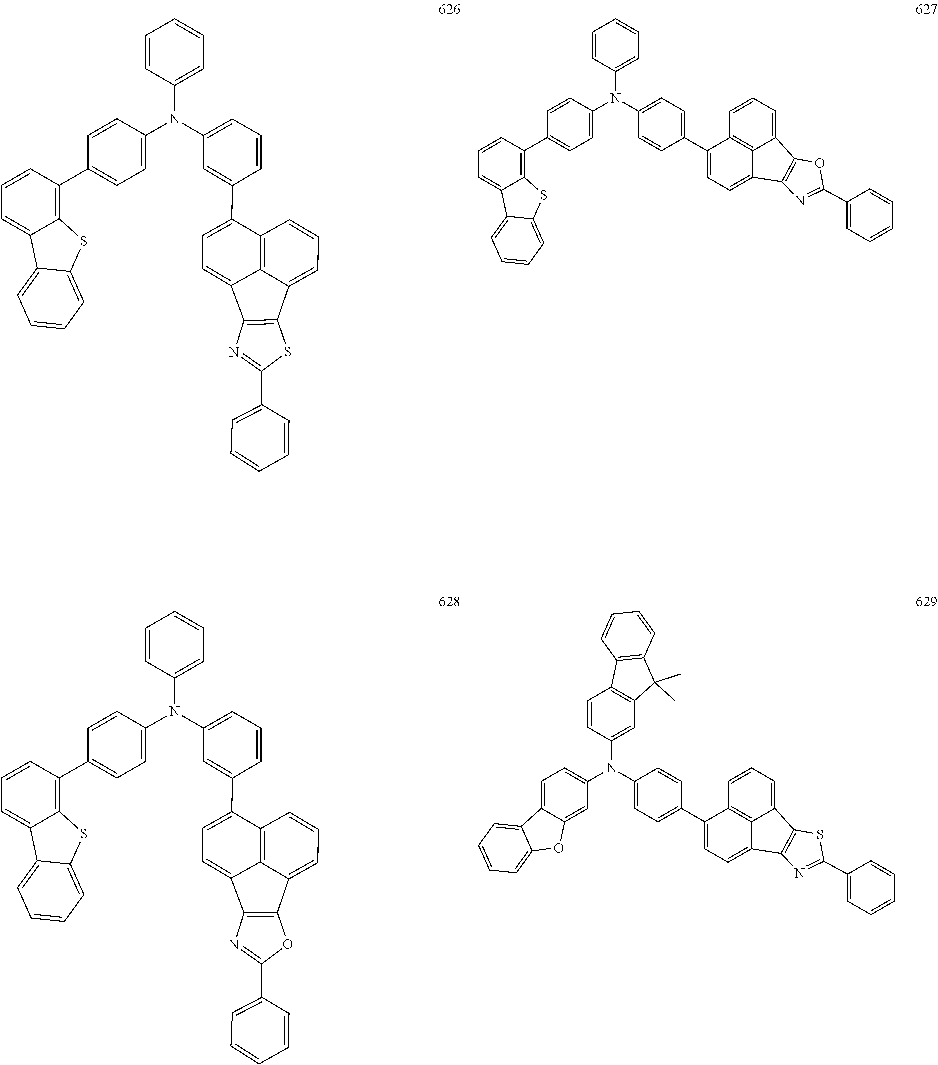 [SMILES: c1ccc(-c2nc3c(s2)-c2cccc4c(-c5cccc(N(c6ccccc6)c6ccc(-c7cccc8c7sc7ccccc78)cc6)c5)ccc-3c24)cc1 ; c1ccc(-c2nc3c(o2)-c2cccc4c(-c5ccc(N(c6ccccc6)c6ccc(-c7cccc8c7sc7ccccc78)cc6)cc5)ccc-3c24)cc1 ; c1ccc(-c2nc3c(o2)-c2cccc4c(-c5cccc(N(c6ccccc6)c6ccc(-c7cccc8c7sc7ccccc78)cc6)c5)ccc-3c24)cc1 ; CC1(C)c2ccccc2-c2ccc(N(c3ccc(-c4ccc5c6c(cccc46)-c4sc(-c6ccccc6)nc4-5)cc3)c3ccc4c(c3)oc3ccccc34)cc21]

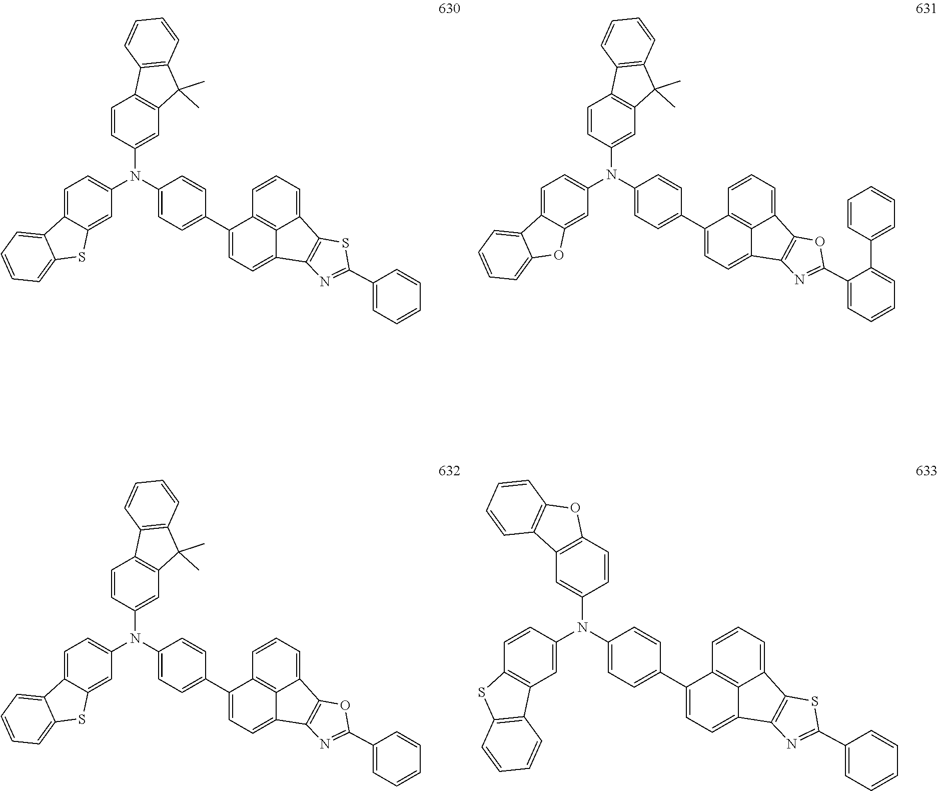 [SMILES: CC1(C)c2ccccc2-c2ccc(N(c3ccc(-c4ccc5c6c(cccc46)-c4sc(-c6ccccc6)nc4-5)cc3)c3ccc4c(c3)sc3ccccc34)cc21 ; CC1(C)c2ccccc2-c2ccc(N(c3ccc(-c4ccc5c6c(cccc46)-c4oc(-c6ccccc6-c6ccccc6)nc4-5)cc3)c3ccc4c(c3)oc3ccccc34)cc21 ; CC1(C)c2ccccc2-c2ccc(N(c3ccc(-c4ccc5c6c(cccc46)-c4oc(-c6ccccc6)nc4-5)cc3)c3ccc4c(c3)sc3ccccc34)cc21 ; c1ccc(-c2nc3c(s2)-c2cccc4c(-c5ccc(N(c6ccc7oc8ccccc8c7c6)c6ccc7sc8ccccc8c7c6)cc5)ccc-3c24)cc1]

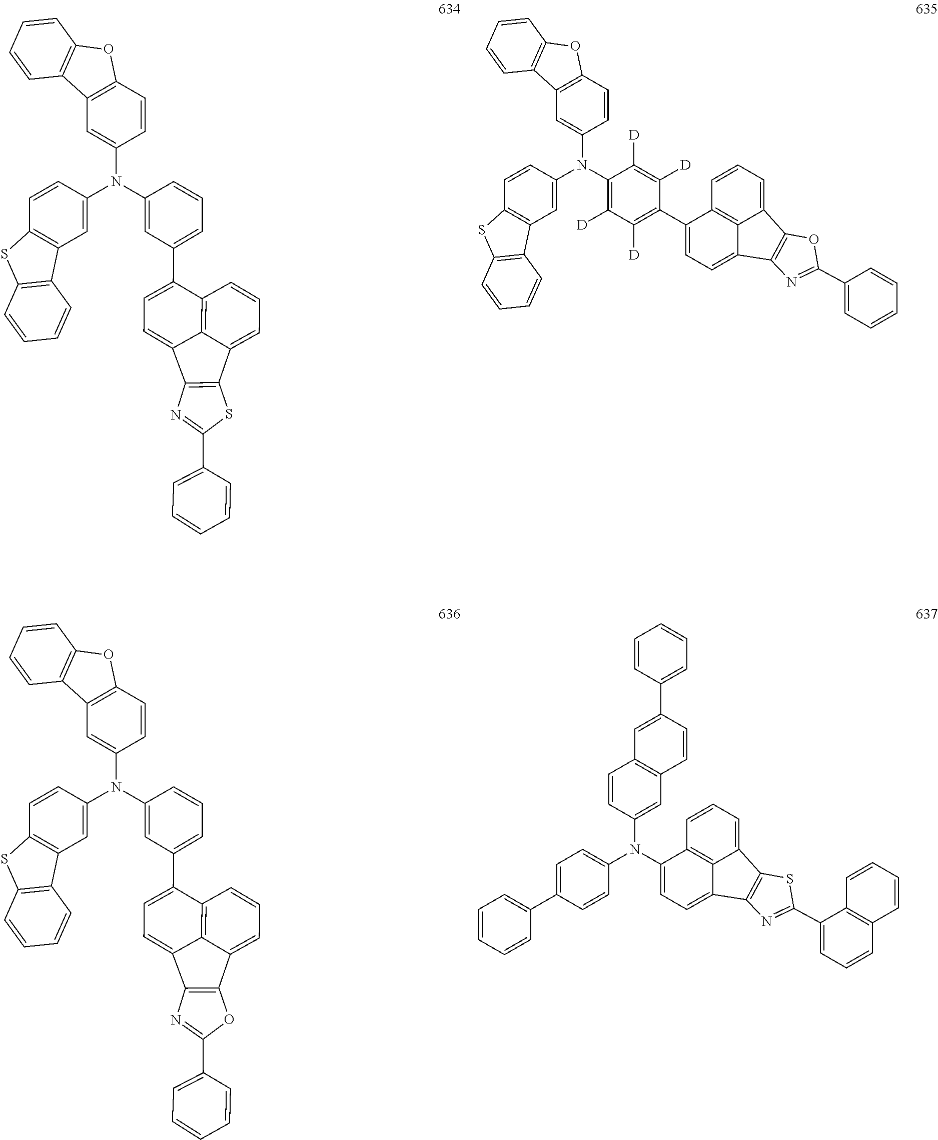 [SMILES: c1ccc(-c2nc3c(s2)-c2cccc4c(-c5cccc(N(c6ccc7oc8ccccc8c7c6)c6ccc7sc8ccccc8c7c6)c5)ccc-3c24)cc1 ; [2H]c1c([2H])c(N(c2ccc3oc4ccccc4c3c2)c2ccc3sc4ccccc4c3c2)c([2H])c([2H])c1-c1ccc2c3c(cccc13)-c1oc(-c3ccccc3)nc1-2 ; c1ccc(-c2nc3c(o2)-c2cccc4c(-c5cccc(N(c6ccc7oc8ccccc8c7c6)c6ccc7sc8ccccc8c7c6)c5)ccc-3c24)cc1 ; c1ccc(-c2ccc(N(c3ccc4cc(-c5ccccc5)ccc4c3)c3ccc4c5c(cccc35)-c3sc(-c5cccc6ccccc56)nc3-4)cc2)cc1]

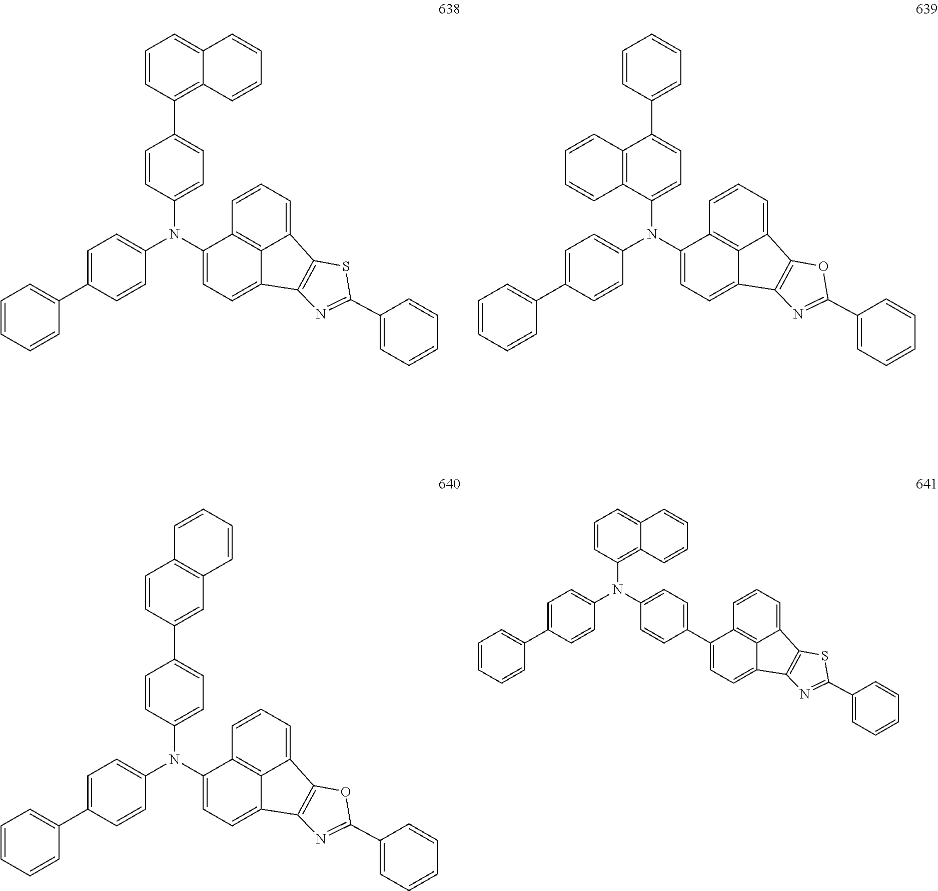

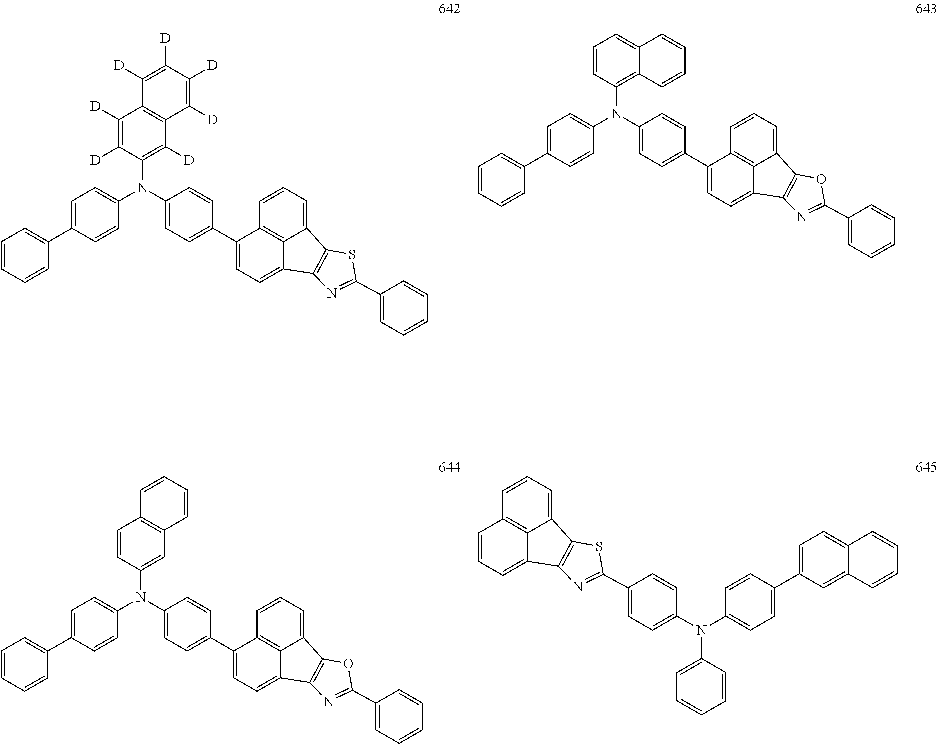 [SMILES: [2H]c1c([2H])c([2H])c2c([2H])c(N(c3ccc(-c4ccccc4)cc3)c3ccc(-c4ccc5c6c(cccc46)-c4sc(-c6ccccc6)nc4-5)cc3)c([2H])c([2H])c2c1[2H] ; c1ccc(-c2ccc(N(c3ccc(-c4ccc5c6c(cccc46)-c4oc(-c6ccccc6)nc4-5)cc3)c3cccc4ccccc34)cc2)cc1 ; c1ccc(-c2ccc(N(c3ccc(-c4ccc5c6c(cccc46)-c4oc(-c6ccccc6)nc4-5)cc3)c3ccc4ccccc4c3)cc2)cc1 ; c1ccc(N(c2ccc(-c3ccc4ccccc4c3)cc2)c2ccc(-c3nc4c(s3)-c3cccc5cccc-4c35)cc2)cc1]

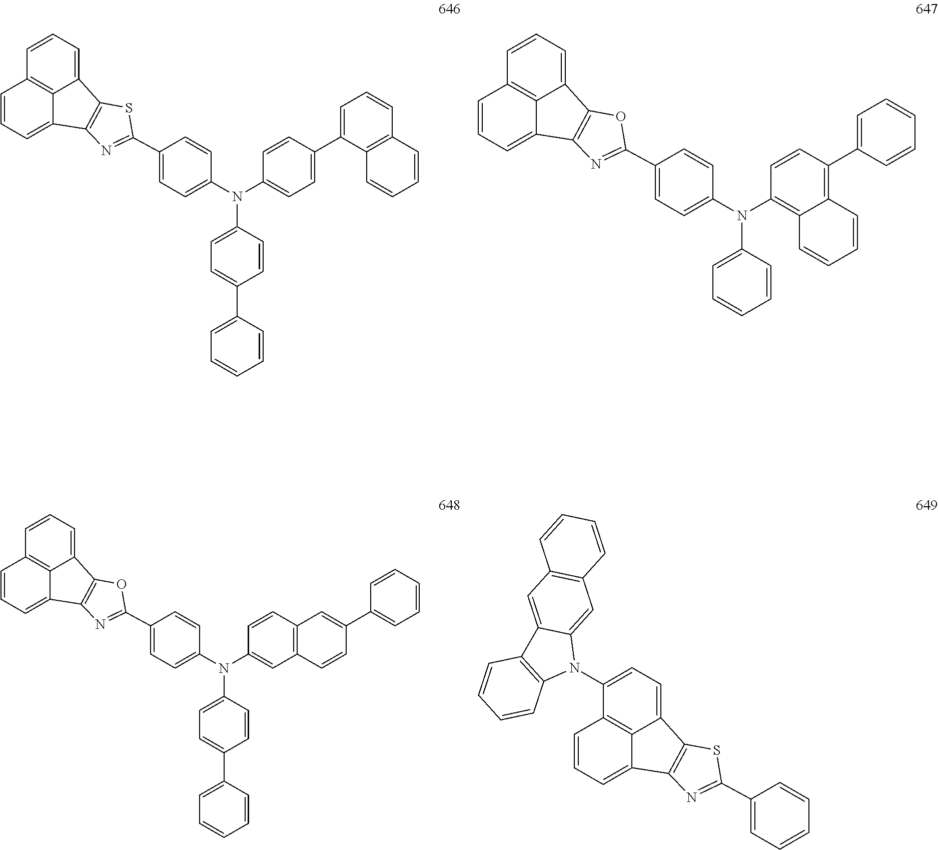 [SMILES: c1ccc(-c2ccc(N(c3ccc(-c4nc5c(s4)-c4cccc6cccc-5c46)cc3)c3ccc(-c4cccc5ccccc45)cc3)cc2)cc1 ; c1ccc(-c2ccc(N(c3ccccc3)c3ccc(-c4nc5c(o4)-c4cccc6cccc-5c46)cc3)c3ccccc23)cc1 ; c1ccc(-c2ccc(N(c3ccc(-c4nc5c(o4)-c4cccc6cccc-5c46)cc3)c3ccc4cc(-c5ccccc5)ccc4c3)cc2)cc1 ; c1ccc(-c2nc3c(s2)-c2ccc(-n4c5ccccc5c5cc6ccccc6cc54)c4cccc-3c24)cc1]

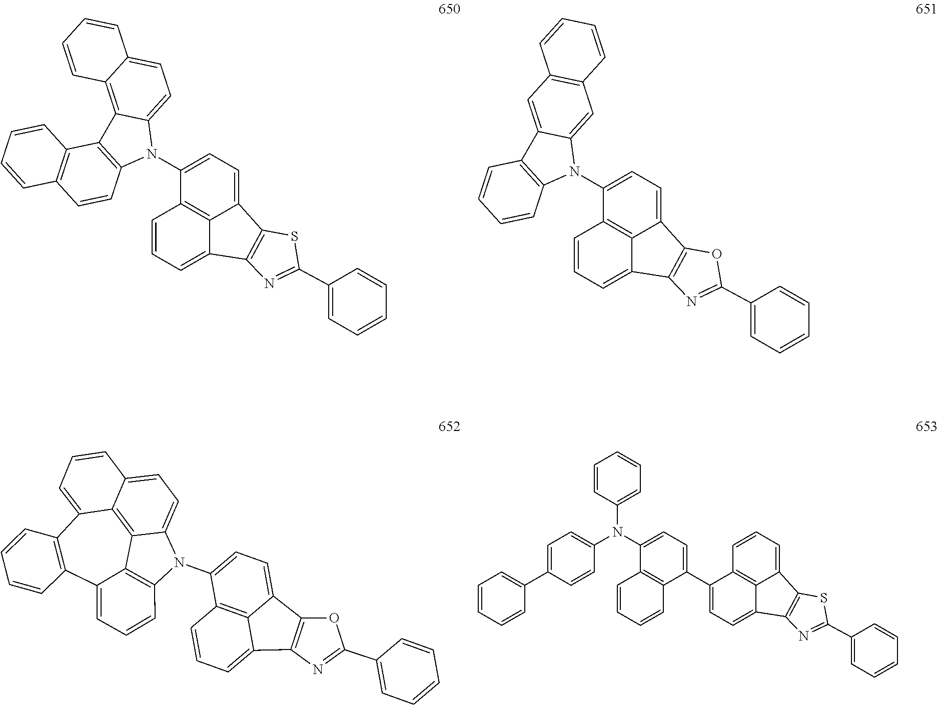 [SMILES: c1ccc(-c2nc3c(s2)-c2ccc(-n4c5ccc6ccccc6c5c5c6ccccc6ccc54)c4cccc-3c24)cc1 ; c1ccc(-c2nc3c(o2)-c2ccc(-n4c5ccccc5c5cc6ccccc6cc54)c4cccc-3c24)cc1 ; c1ccc(-c2nc3c(o2)-c2ccc(-n4c5cccc6c5c5c7c(cccc7ccc54)-c4ccccc4-6)c4cccc-3c24)cc1 ; c1ccc(-c2ccc(N(c3ccccc3)c3ccc(-c4ccc5c6c(cccc46)-c4sc(-c6ccccc6)nc4-5)c4ccccc34)cc2)cc1]

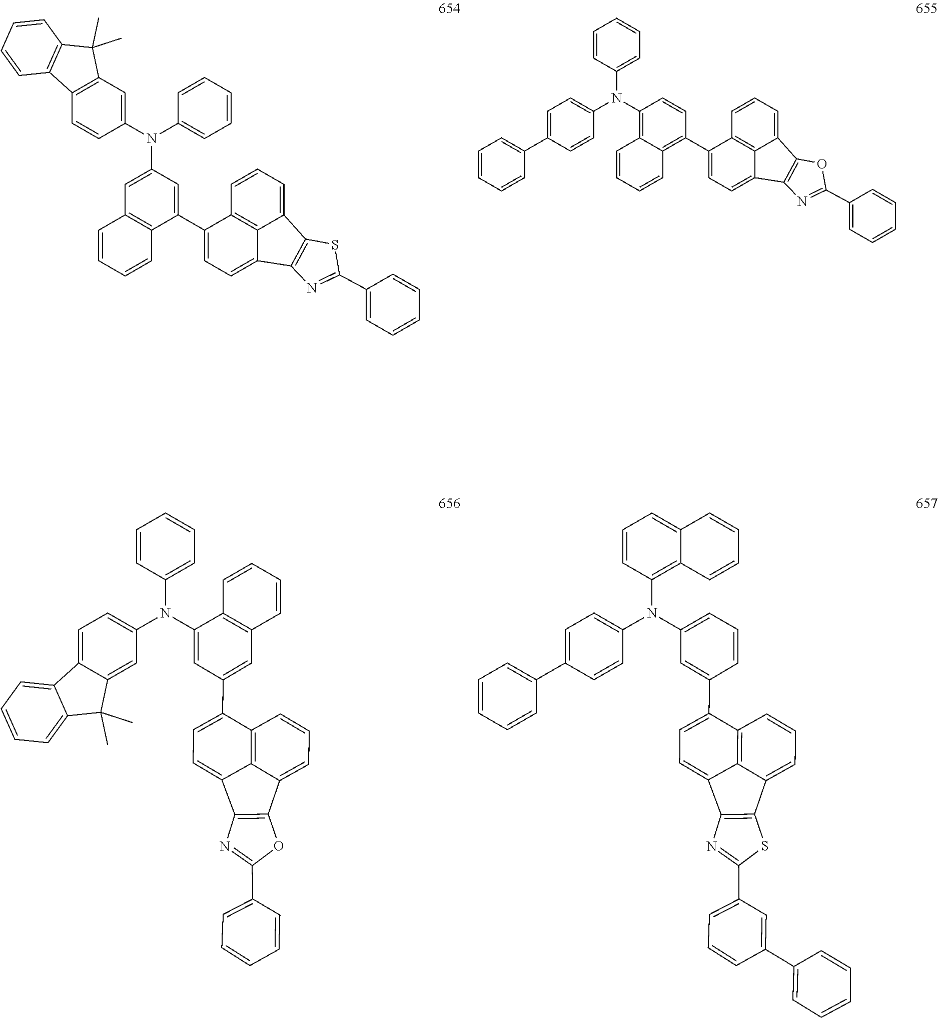 [SMILES: CC1(C)c2ccccc2-c2ccc(N(c3ccccc3)c3cc(-c4ccc5c6c(cccc46)-c4sc(-c6ccccc6)nc4-5)c4ccccc4c3)cc21 ; c1ccc(-c2ccc(N(c3ccccc3)c3ccc(-c4ccc5c6c(cccc46)-c4oc(-c6ccccc6)nc4-5)c4ccccc34)cc2)cc1 ; CC1(C)c2ccccc2-c2ccc(N(c3ccccc3)c3cc(-c4ccc5c6c(cccc46)-c4oc(-c6ccccc6)nc4-5)cc4ccccc34)cc21 ; c1ccc(-c2ccc(N(c3cccc(-c4ccc5c6c(cccc46)-c4sc(-c6cccc(-c7ccccc7)c6)nc4-5)c3)c3cccc4ccccc34)cc2)cc1]

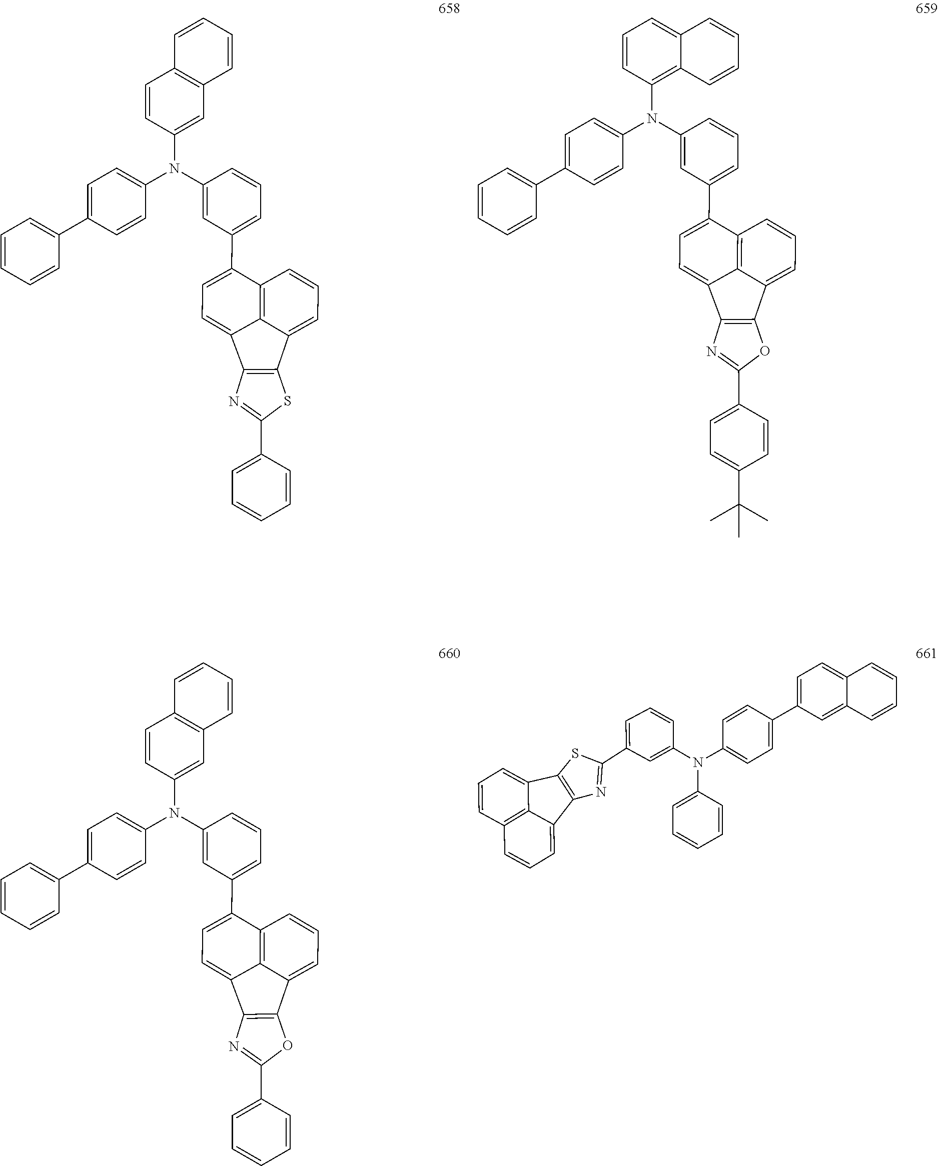 [SMILES: c1ccc(-c2ccc(N(c3cccc(-c4ccc5c6c(cccc46)-c4sc(-c6ccccc6)nc4-5)c3)c3ccc4ccccc4c3)cc2)cc1 ; CC(C)(C)c1ccc(-c2nc3c(o2)-c2cccc4c(-c5cccc(N(c6ccc(-c7ccccc7)cc6)c6cccc7ccccc67)c5)ccc-3c24)cc1 ; c1ccc(-c2ccc(N(c3cccc(-c4ccc5c6c(cccc46)-c4oc(-c6ccccc6)nc4-5)c3)c3ccc4ccccc4c3)cc2)cc1 ; c1ccc(N(c2ccc(-c3ccc4ccccc4c3)cc2)c2cccc(-c3nc4c(s3)-c3cccc5cccc-4c35)c2)cc1]

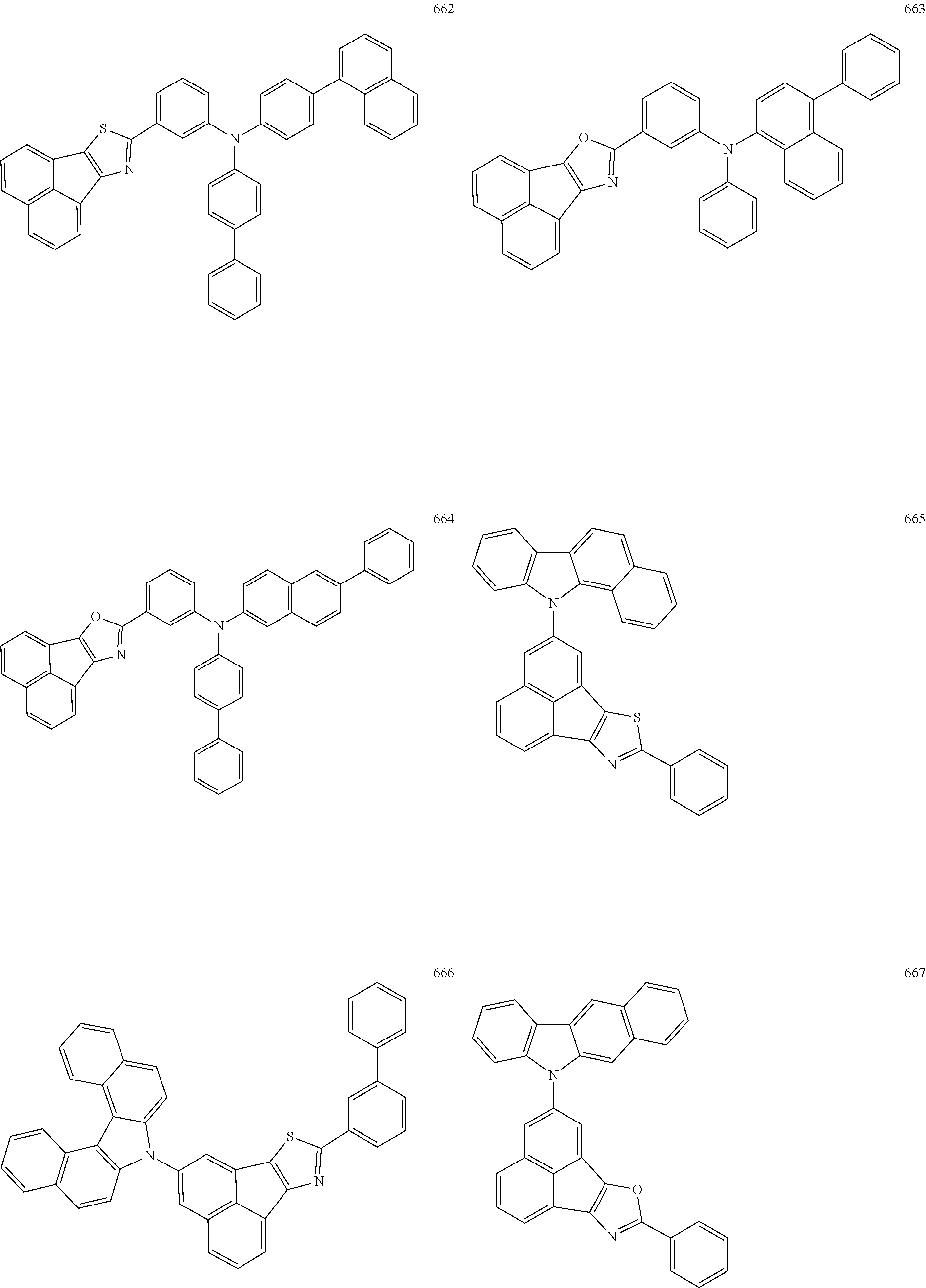 [SMILES: c1ccc(-c2ccc(N(c3ccc(-c4cccc5ccccc45)cc3)c3cccc(-c4nc5c(s4)-c4cccc6cccc-5c46)c3)cc2)cc1 ; c1ccc(-c2ccc(N(c3ccccc3)c3cccc(-c4nc5c(o4)-c4cccc6cccc-5c46)c3)c3ccccc23)cc1 ; c1ccc(-c2ccc(N(c3cccc(-c4nc5c(o4)-c4cccc6cccc-5c46)c3)c3ccc4cc(-c5ccccc5)ccc4c3)cc2)cc1 ; c1ccc(-c2nc3c(s2)-c2cc(-n4c5ccccc5c5ccc6ccccc6c54)cc4cccc-3c24)cc1 ; c1ccc(-c2cccc(-c3nc4c(s3)-c3cc(-n5c6ccc7ccccc7c6c6c7ccccc7ccc65)cc5cccc-4c35)c2)cc1 ; c1ccc(-c2nc3c(o2)-c2cc(-n4c5ccccc5c5cc6ccccc6cc54)cc4cccc-3c24)cc1]

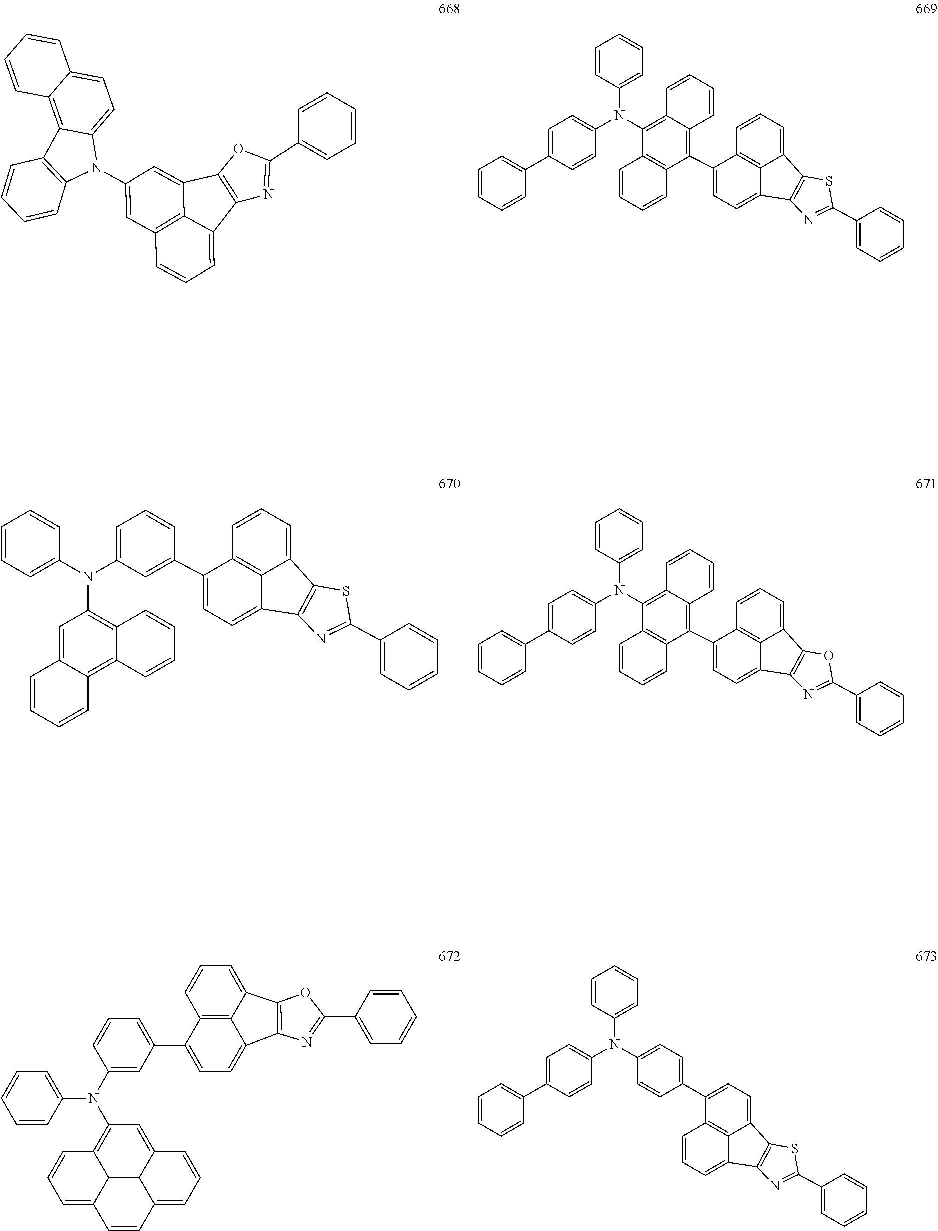

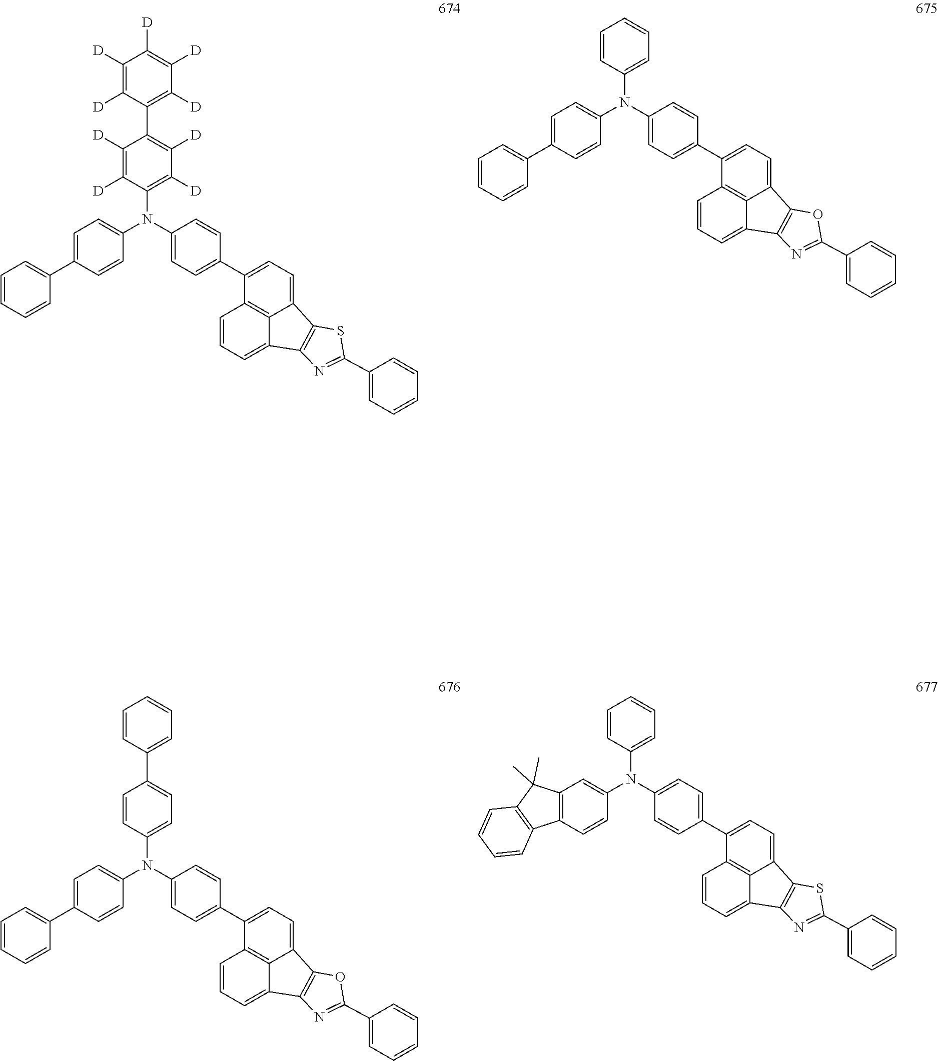 [SMILES: [2H]c1c([2H])c([2H])c(-c2c([2H])c([2H])c(N(c3ccc(-c4ccccc4)cc3)c3ccc(-c4ccc5c6c(cccc46)-c4nc(-c6ccccc6)sc4-5)cc3)c([2H])c2[2H])c([2H])c1[2H] ; c1ccc(-c2ccc(N(c3ccccc3)c3ccc(-c4ccc5c6c(cccc46)-c4nc(-c6ccccc6)oc4-5)cc3)cc2)cc1 ; c1ccc(-c2ccc(N(c3ccc(-c4ccccc4)cc3)c3ccc(-c4ccc5c6c(cccc46)-c4nc(-c6ccccc6)oc4-5)cc3)cc2)cc1 ; CC1(C)c2ccccc2-c2ccc(N(c3ccccc3)c3ccc(-c4ccc5c6c(cccc46)-c4nc(-c6ccccc6)sc4-5)cc3)cc21]

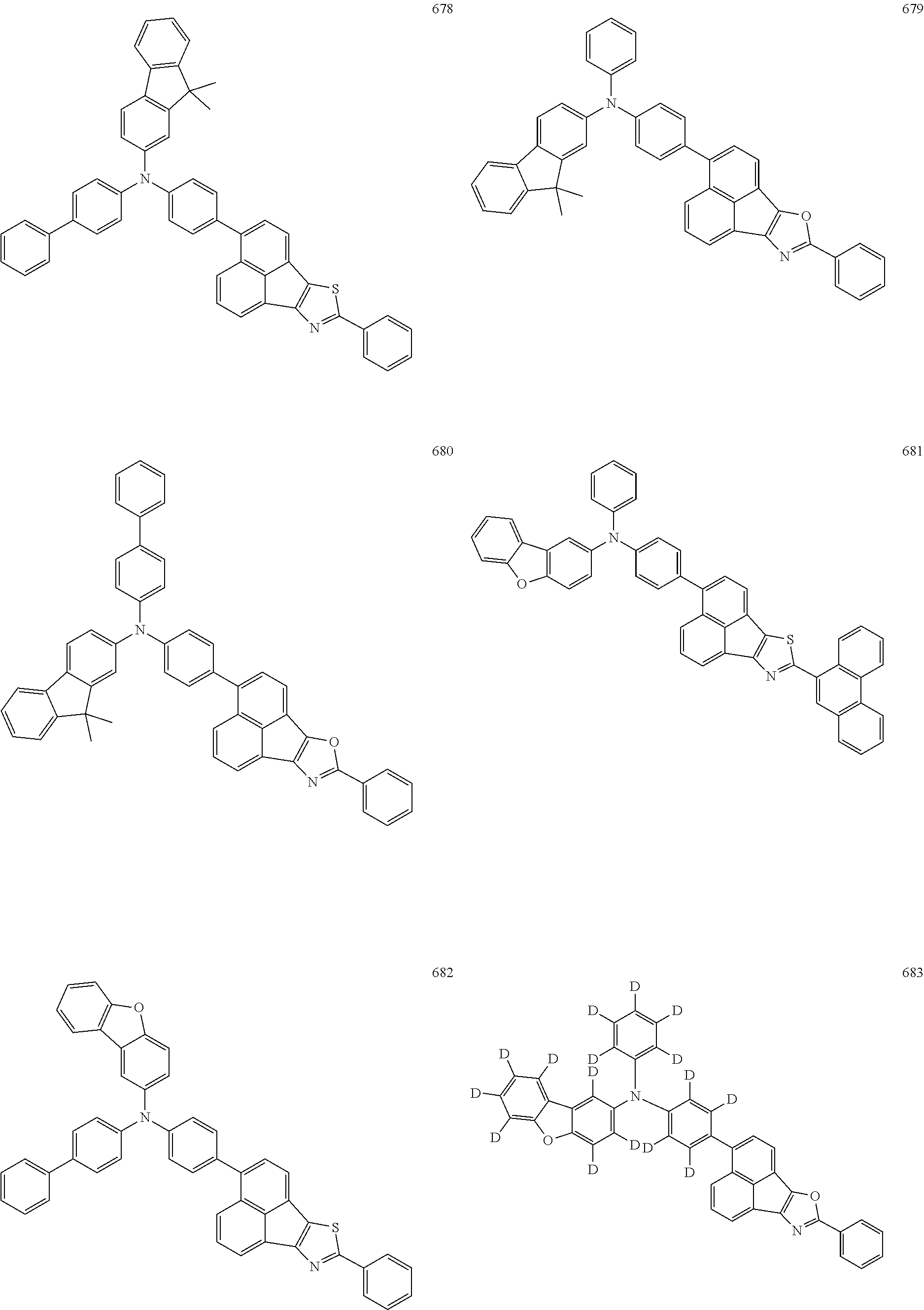

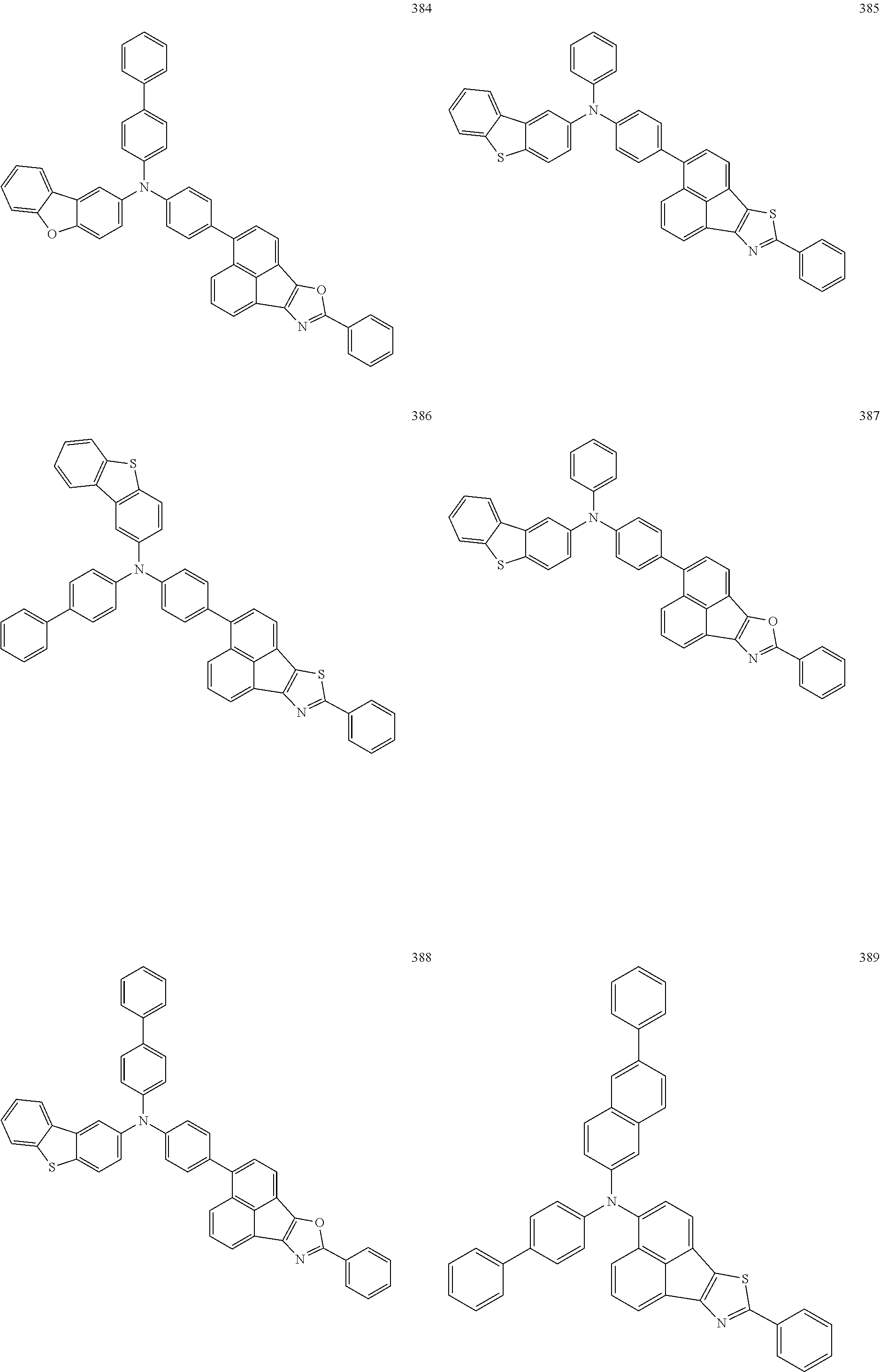

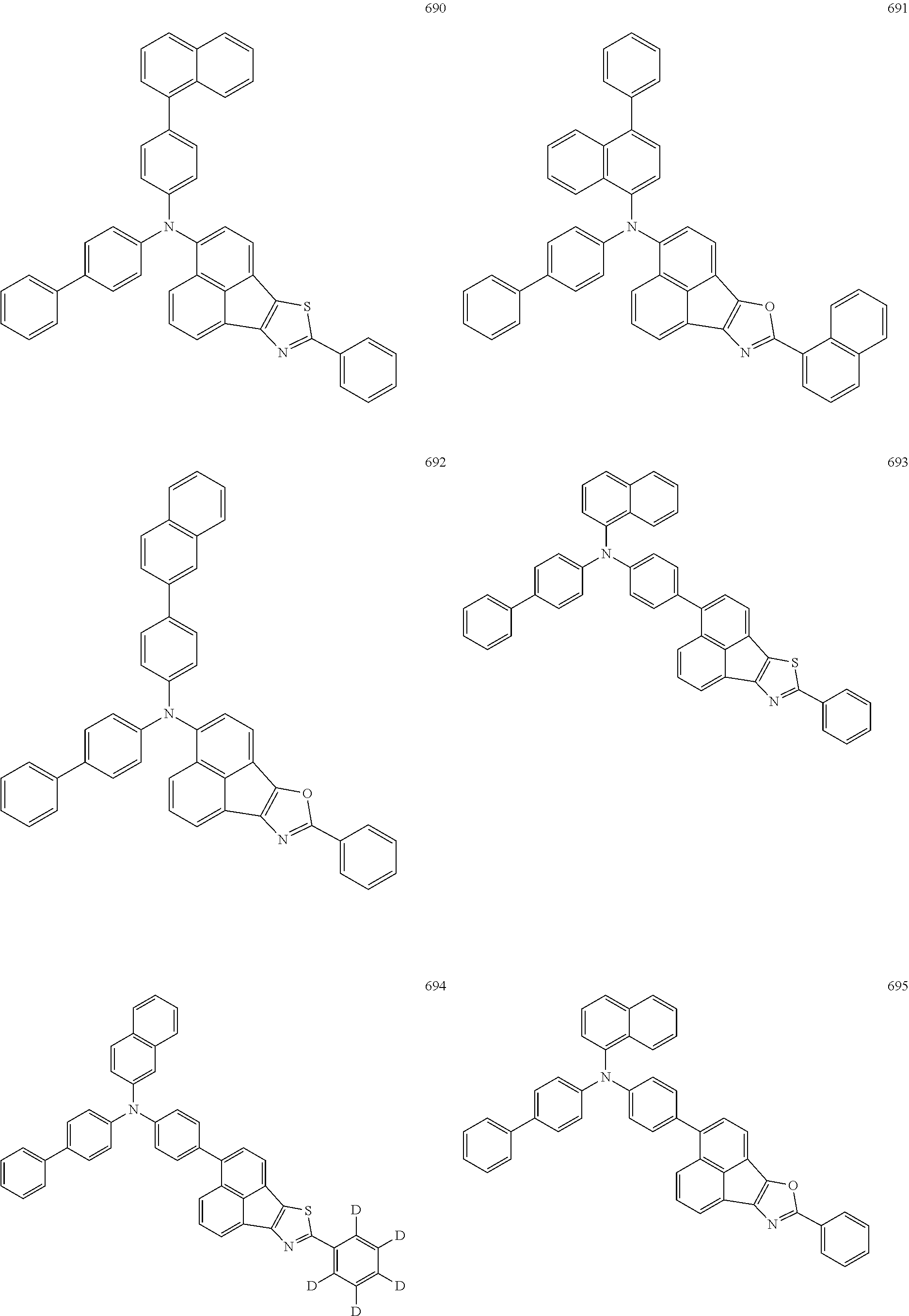

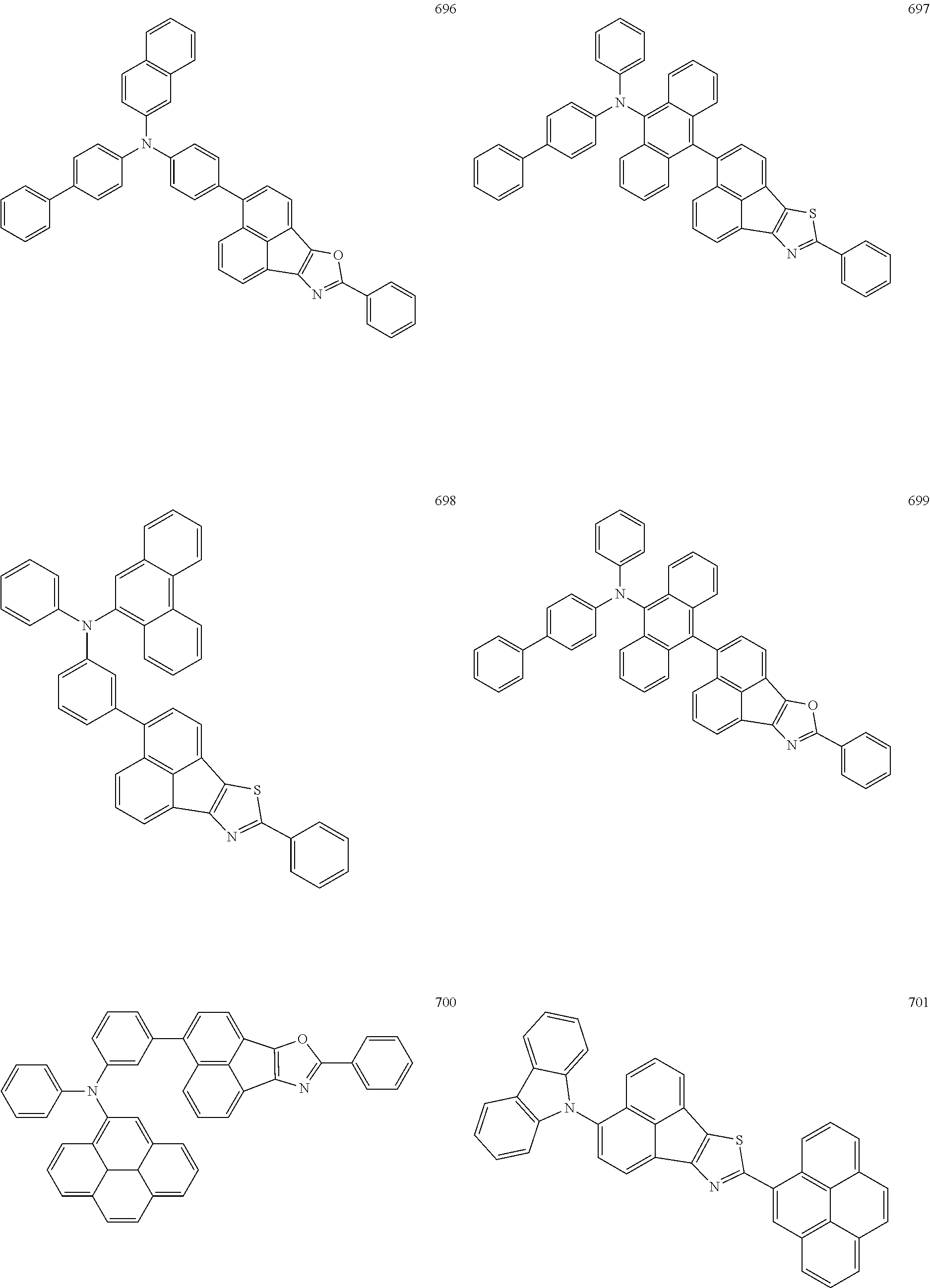

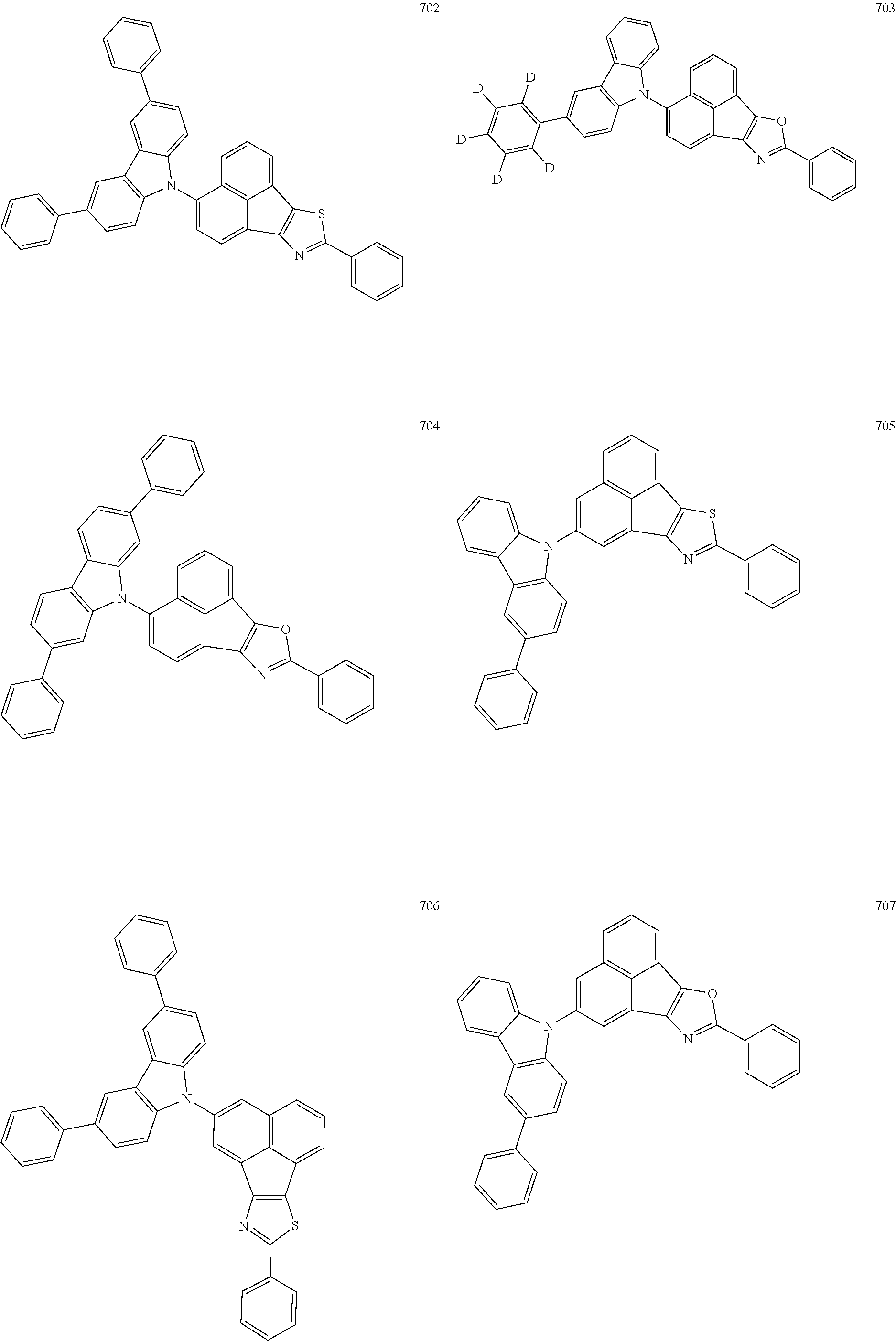 [SMILES: c1ccc(-c2ccc3c(c2)c2cc(-c4ccccc4)ccc2n3-c2ccc3c4c(cccc24)-c2sc(-c4ccccc4)nc2-3)cc1 ; [2H]c1c([2H])c([2H])c(-c2ccc3c(c2)c2ccccc2n3-c2ccc3c4c(cccc24)-c2oc(-c4ccccc4)nc2-3)c([2H])c1[2H] ; c1ccc(-c2ccc3c4ccc(-c5ccccc5)cc4n(-c4ccc5c6c(cccc46)-c4oc(-c6ccccc6)nc4-5)c3c2)cc1 ; c1ccc(-c2ccc3c(c2)c2ccccc2n3-c2cc3c4c(cccc4c2)-c2sc(-c4ccccc4)nc2-3)cc1 ; c1ccc(-c2ccc3c(c2)c2cc(-c4ccccc4)ccc2n3-c2cc3c4c(cccc4c2)-c2sc(-c4ccccc4)nc2-3)cc1 ; c1ccc(-c2ccc3c(c2)c2ccccc2n3-c2cc3c4c(cccc4c2)-c2oc(-c4ccccc4)nc2-3)cc1]

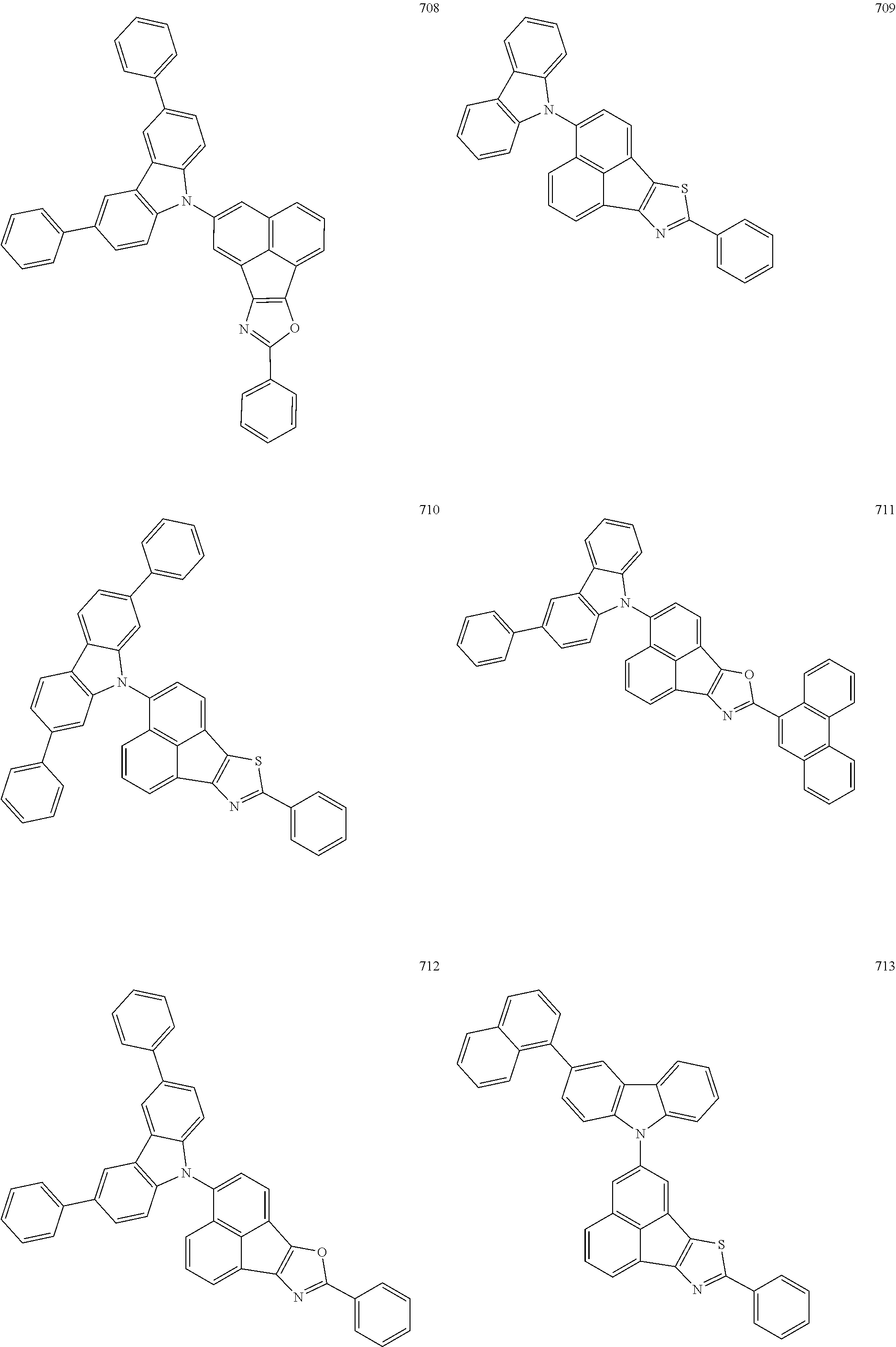 [SMILES: c1ccc(-c2ccc3c(c2)c2cc(-c4ccccc4)ccc2n3-c2cc3c4c(cccc4c2)-c2oc(-c4ccccc4)nc2-3)cc1 ; c1ccc(-c2nc3c(s2)-c2ccc(-n4c5ccccc5c5ccccc54)c4cccc-3c24)cc1 ; c1ccc(-c2ccc3c4ccc(-c5ccccc5)cc4n(-c4ccc5c6c(cccc46)-c4nc(-c6ccccc6)sc4-5)c3c2)cc1 ; c1ccc(-c2ccc3c(c2)c2ccccc2n3-c2ccc3c4c(cccc24)-c2nc(-c4cc5ccccc5c5ccccc45)oc2-3)cc1 ; c1ccc(-c2ccc3c(c2)c2cc(-c4ccccc4)ccc2n3-c2ccc3c4c(cccc24)-c2nc(-c4ccccc4)oc2-3)cc1 ; c1ccc(-c2nc3c(s2)-c2cc(-n4c5ccccc5c5cc(-c6cccc7ccccc67)ccc54)cc4cccc-3c24)cc1]

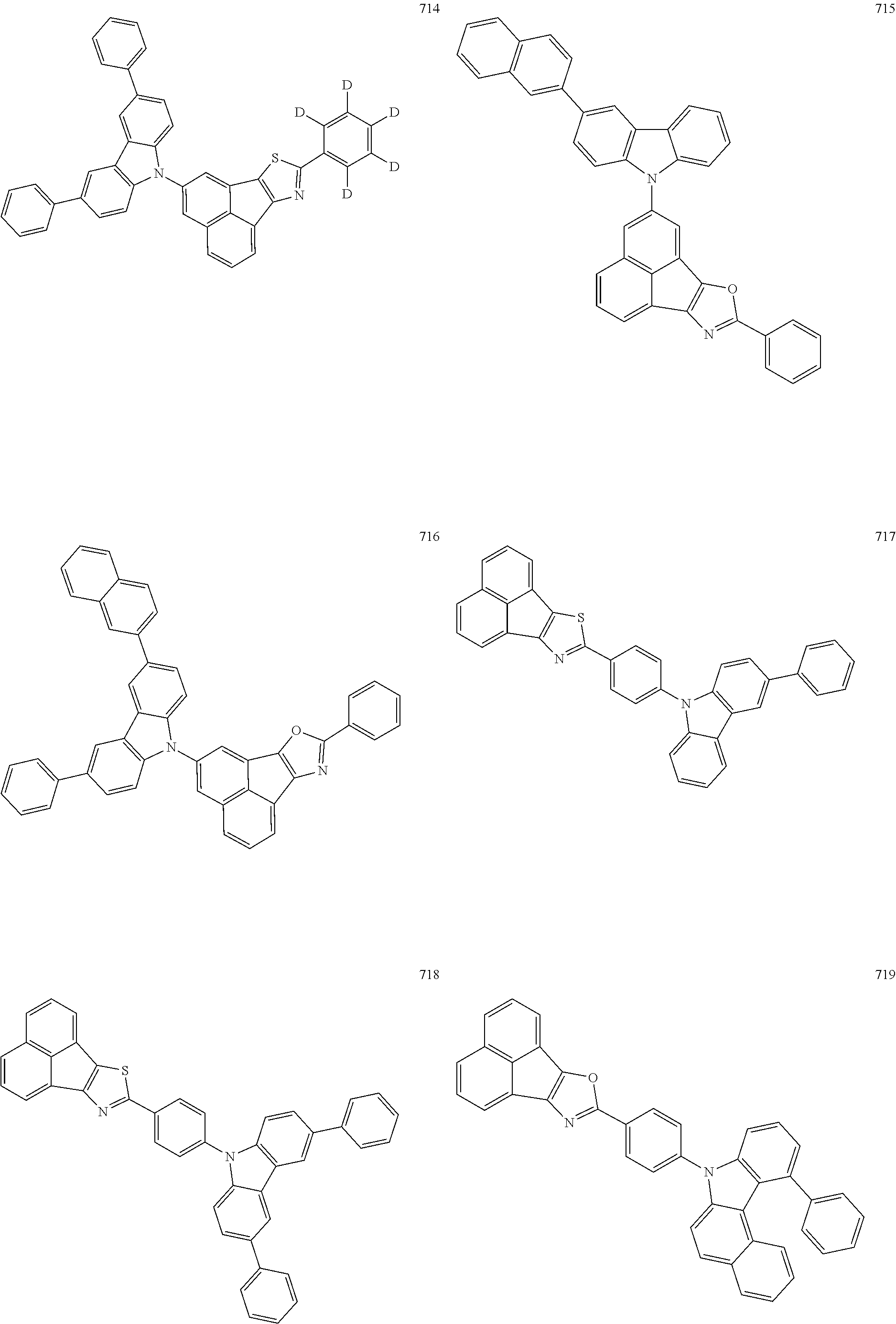

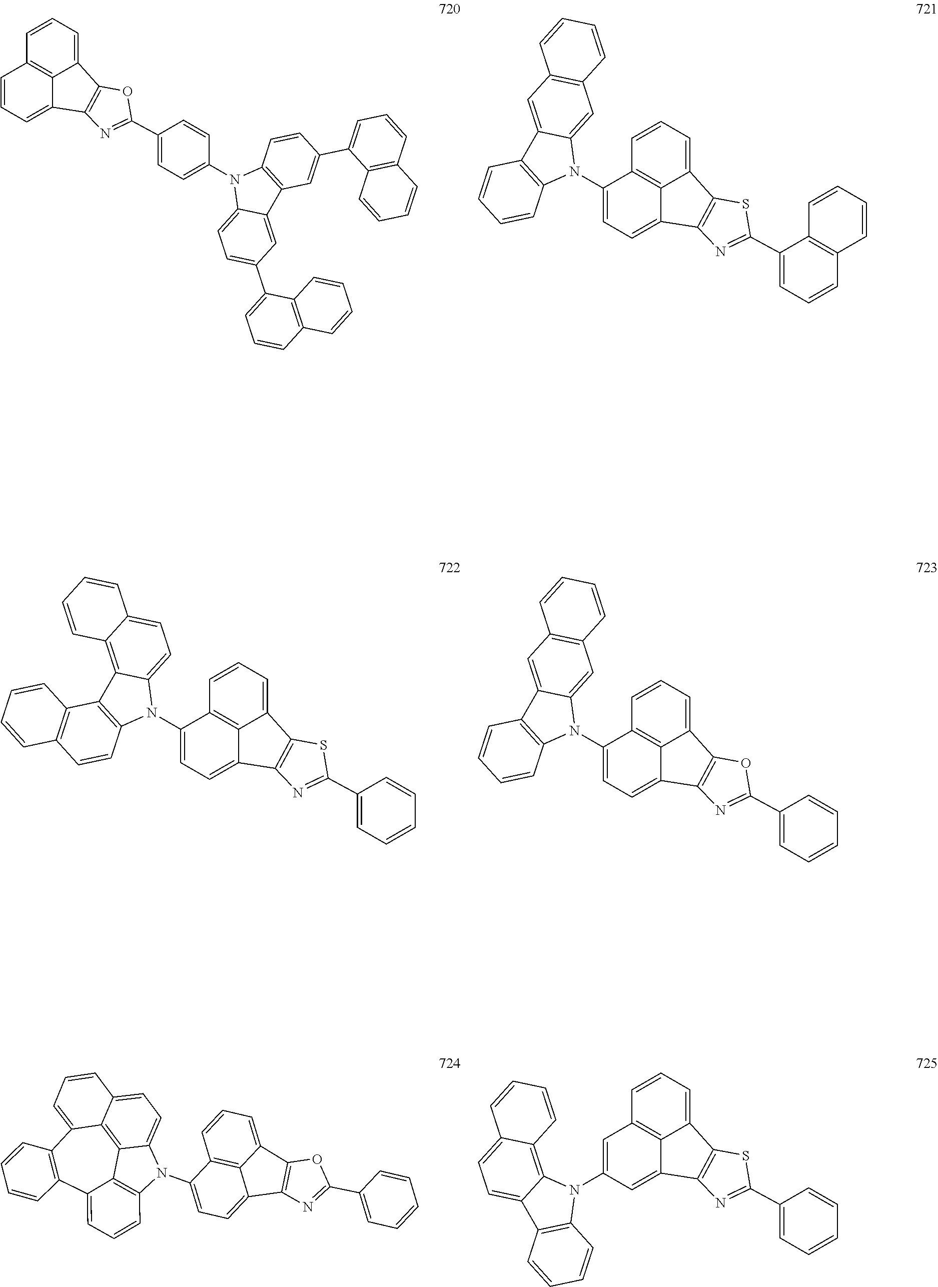 [SMILES: c1ccc2c(-c3ccc4c(c3)c3cc(-c5cccc6ccccc56)ccc3n4-c3ccc(-c4nc5c(o4)-c4cccc6cccc-5c46)cc3)cccc2c1 ; c1ccc2cc3c(cc2c1)c1ccccc1n3-c1ccc2c3c(cccc13)-c1sc(-c3cccc4ccccc34)nc1-2 ; c1ccc(-c2nc3c(s2)-c2cccc4c(-n5c6ccc7ccccc7c6c6c7ccccc7ccc65)ccc-3c24)cc1 ; c1ccc(-c2nc3c(o2)-c2cccc4c(-n5c6ccccc6c6cc7ccccc7cc65)ccc-3c24)cc1 ; c1ccc(-c2nc3c(o2)-c2cccc4c(-n5c6cccc7c6c6c8c(cccc8ccc65)-c5ccccc5-7)ccc-3c24)cc1 ; c1ccc(-c2nc3c(s2)-c2cccc4cc(-n5c6ccccc6c6ccc7ccccc7c65)cc-3c24)cc1]

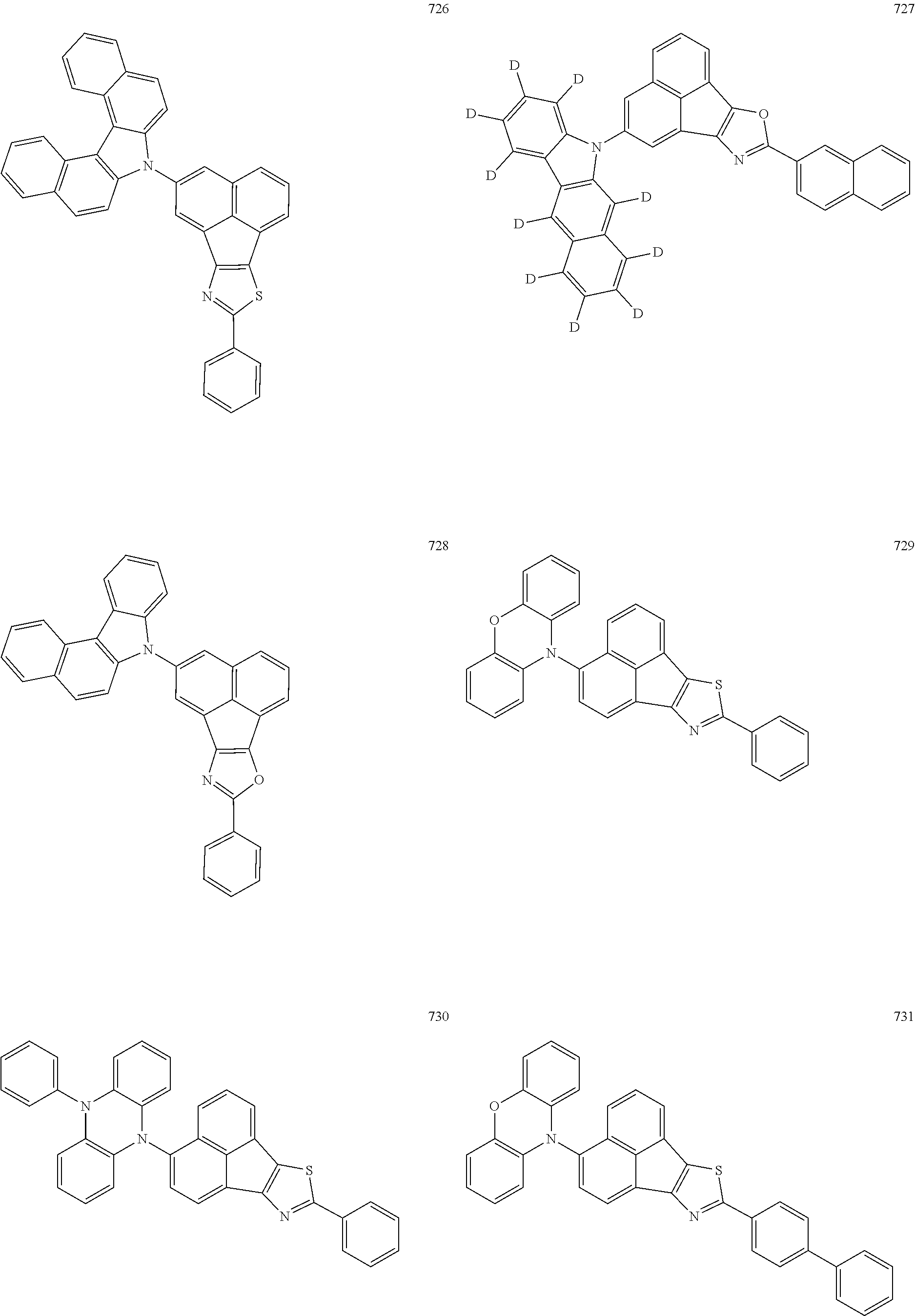 [SMILES: c1ccc(-c2nc3c(s2)-c2cccc4cc(-n5c6ccc7ccccc7c6c6c7ccccc7ccc65)cc-3c24)cc1 ; [2H]c1c([2H])c([2H])c2c([2H])c3c(c([2H])c2c1[2H])c1c([2H])c([2H])c([2H])c([2H])c1n3-c1cc2c3c(cccc3c1)-c1oc(-c3ccc4ccccc4c3)nc1-2 ; c1ccc(-c2nc3c(o2)-c2cccc4cc(-n5c6ccccc6c6c7ccccc7ccc65)cc-3c24)cc1 ; c1ccc(-c2nc3c(s2)-c2cccc4c(N5c6ccccc6Oc6ccccc65)ccc-3c24)cc1 ; c1ccc(-c2nc3c(s2)-c2cccc4c(N5c6ccccc6N(c6ccccc6)c6ccccc65)ccc-3c24)cc1 ; c1ccc(-c2ccc(-c3nc4c(s3)-c3cccc5c(N6c7ccccc7Oc7ccccc76)ccc-4c35)cc2)cc1]

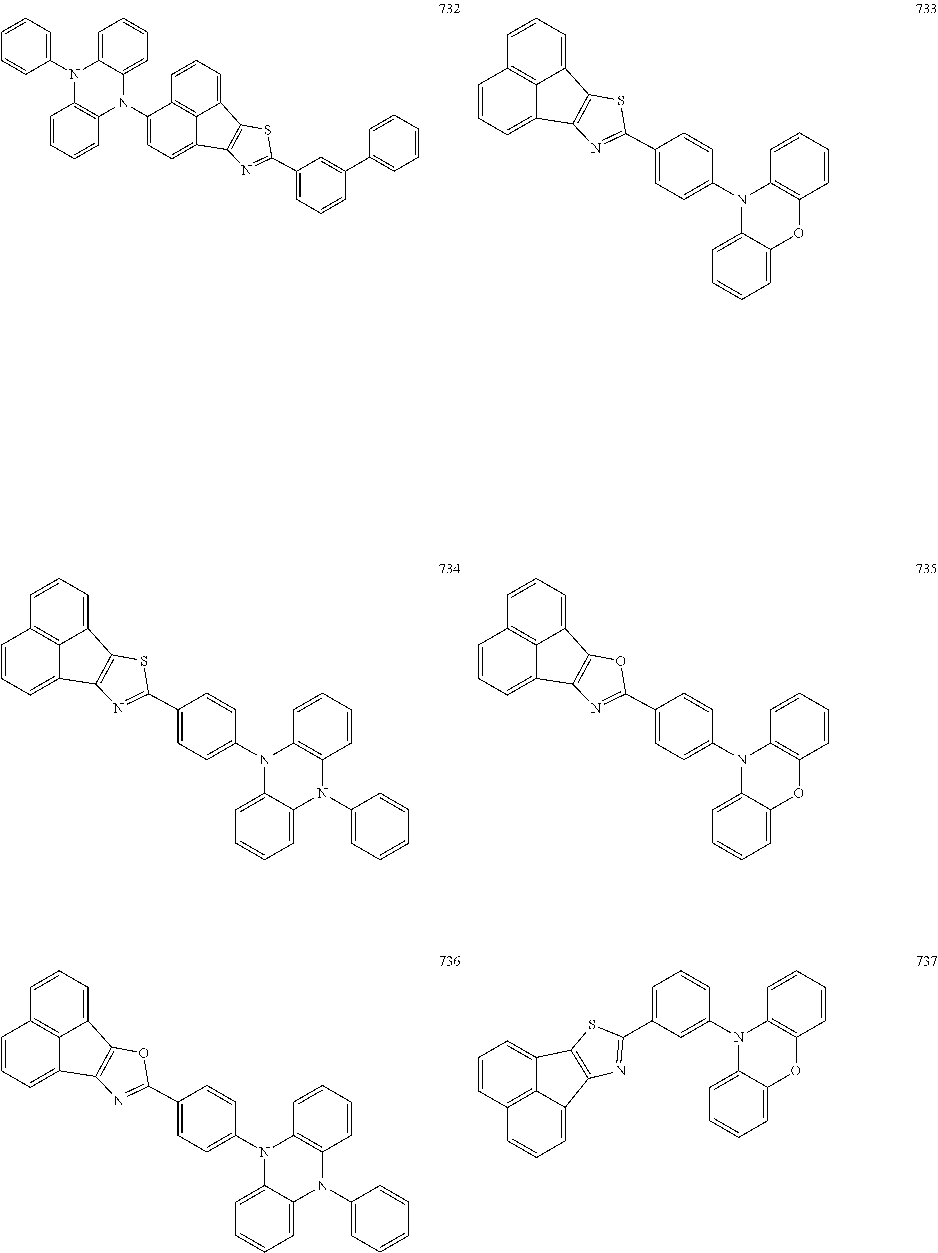 [SMILES: c1ccc(-c2cccc(-c3nc4c(s3)-c3cccc5c(N6c7ccccc7N(c7ccccc7)c7ccccc76)ccc-4c35)c2)cc1 ; c1ccc2c(c1)Oc1ccccc1N2c1ccc(-c2nc3c(s2)-c2cccc4cccc-3c24)cc1 ; c1ccc(N2c3ccccc3N(c3ccc(-c4nc5c(s4)-c4cccc6cccc-5c46)cc3)c3ccccc32)cc1 ; c1ccc2c(c1)Oc1ccccc1N2c1ccc(-c2nc3c(o2)-c2cccc4cccc-3c24)cc1 ; c1ccc(N2c3ccccc3N(c3ccc(-c4nc5c(o4)-c4cccc6cccc-5c46)cc3)c3ccccc32)cc1 ; c1cc(-c2nc3c(s2)-c2cccc4cccc-3c24)cc(N2c3ccccc3Oc3ccccc32)c1]

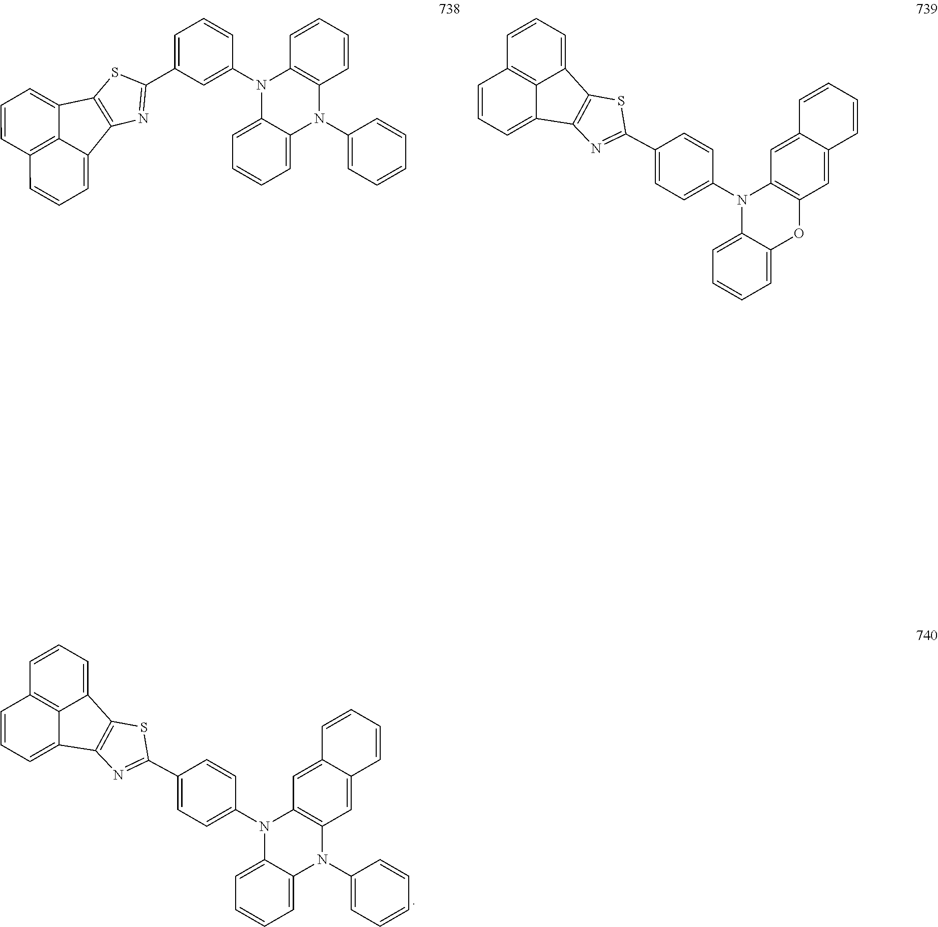 [SMILES: c1ccc(N2c3ccccc3N(c3cccc(-c4nc5c(s4)-c4cccc6cccc-5c46)c3)c3ccccc32)cc1 ; c1ccc2c(c1)Oc1cc3ccccc3cc1N2c1ccc(-c2nc3c(s2)-c2cccc4cccc-3c24)cc1 ; c1ccc(N2c3ccccc3N(c3ccc(-c4nc5c(s4)-c4cccc6cccc-5c46)cc3)c3cc4ccccc4cc32)cc1]

11. An organic light emitting device comprising:
a first electrode;
a second electrode provided opposite to the first electrode; and
one or more organic material layers provided between the first electrode and the second electrode,
wherein one or more layers of the one or more organic material layers comprise the heterocyclic compound of claim 1.

12. The organic light emitting device of claim 11, wherein the one or more organic material layer comprise a light emitting layer, and the light emitting layer comprises the heterocyclic compound.

13. The organic light emitting device of claim 11, wherein the one or more organic material layer comprise a light emitting layer, and the light emitting layer comprises the heterocyclic compound as a host material.

14. The organic light emitting device of claim 11, further comprising one or more selected from the group consisting of a light emitting layer, a hole injection layer, a hole transport layer, an electron injection layer, an electron transport layer, an electron blocking layer and a hole blocking layer.
